(19) United States
(12) Patent Application Publication
(10) Pub. No.: US 2024/0294646 A1
(43) Pub. Date: Sep. 5, 2024

(54) HETERODIMERIC Fc VARIANTS SELECTIVE FOR Fc GAMMA RIIB

(71) Applicant: Zymeworks BC Inc., Vancouver (CA)

(72) Inventors: ERIC ESCOBAR-CABRERA, Vancouver, BC (CA); Gregory LAKATOS, Vancouver, BC (CA); Leonard G. PRESTA, Vancouver, BC (CA); Genevieve DESJARDINS, Vancouver, BC (CA); Abhishek MUKHOPADHYAY, Vancouver, BC (CA); Antonios SAMIOTAKIS, Vancouver, BC (CA); Surjit DIXIT, Vancouver, BC (CA); Jian (James) ZHANG, Vancouver, BC (CA); James MCWHIRTER, Vancouver, BC (CA); Gavin JONES, Middlesex (GB)

(21) Appl. No.: 17/999,469

(22) PCT Filed: May 20, 2021
(86) PCT No.: PCT/CA2021/050690

§ 371 Date: Nov. 21, 2022

Related U.S. Application Data
(60) Provisional application No. 63/027,787, filed on May 20, 2020.

(51) Int. Cl. C07K 16/28; A61K 39/00; C07K 16/32; G16B 15/30

(52) U.S. Cl. CPC C07K 16/283; C07K 16/2878; C07K 16/32; G16B 15/30; A61K 2039/505; C07K 2317/524; C07K 2317/72; C07K 2317/734; C07K 2317/92

(57) ABSTRACT

Heterodimeric Fc variants comprising one or more asymmetric amino acid mutations in the CH2 domain and having increased selectivity of binding to FcγRIIb as compared to a parental Fc region, polypeptides comprising the heterodimeric Fc variants and polynucleotides encoding the heterodimeric Fc variants. The one or more asymmetric mutations comprise replacement of a loop in the CH2 domain, a mutation at position 236 in the CH2 domain, or a combination of replacement of a loop in the CH2 domain and a mutation at position 236 in the CH2 domain.

### FIELD

The present disclosure relates to the field of Fc variants and, in particular, to heterodimeric Fc variants with selectivity for FcγRIIb.

### BACKGROUND

The interactions between antibody Fc domains and members of the cellular Fcγ receptor (FcγR) family profoundly influence the strength of the immune response. In the context of therapeutic development, two members of the FcγR family are of particular interest: FcγRIIa, which upregulates immune activity when bound to an antibody Fc, and FcγRIIb, which down-regulates immune activity when bound to an antibody Fc. FcγRIIb is the only inhibitory IgG receptor and down-regulates immune activity by inhibiting the activation of B lymphocytes, monocytes, mast cells and basophils induced by activating receptors.

Fc engineering has been employed to modulate the ability of antibodies to interact with the FcγRs (Carter, 2006, Nat Rev Immunol., 6:343-357; Presta, 2008, Curr Opin Immunol., 20:460-470). Fc engineering to increase affinity and selectivity of the Fc region for FcγRIIb has been described (Chu, et al., 2008, Mol Immunol., 45:3926-3933; Mimoto et al., 2013, Protein Eng. Des. Sel., 26:589-598; U.S. Pat. Nos. 9,540,451; 9,902,773 and 9,914,778; U.S. Patent Application Publication Nos: US 2009/0042291; US 2015/0299296; US 2016/0039912 and US 2016/0046693).

Fc engineering approaches that include inserting additional amino acids into the Fc region to alter FcγR or FcRn binding have also been described (U.S. Pat. No. 9,890,216; U.S. Patent Application Publication Nos: US 2008/0227958 and US 2014/0356358).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are heterodimeric Fc variants selective for FcγRIIb. In one aspect, the present disclosure relates to a heterodimeric Fc variant comprising a first Fc polypeptide and a second Fc polypeptide, the heterodimeric Fc variant having increased selectivity of binding to FcγRIIb as compared to a parental Fc region, wherein one of the Fc polypeptides comprises a replacement of all or a part of a natural loop in the CH2 domain of the Fc polypeptide with an alternative amino acid sequence such that the natural loop is extended in length and at least one of the amino acid residues of the alternative amino acid sequence is within a heavy atom to heavy atom distance of 3 Å of a target amino acid residue in FcγRIIb when the heterodimeric Fc variant is bound by FcγRIIb, and wherein the heterodimeric Fc variant is a variant of an immunoglobulin G (IgG) Fc.

In another aspect, the present disclosure relates to a heterodimeric Fc variant comprising a first Fc polypeptide and a second Fc polypeptide, one of the Fc polypeptides comprising a replacement of amino acids 325 to 331 with a polypeptide between 8 and 15 amino acids in length, wherein the heterodimeric Fc variant has increased selectivity of binding to FcγRIIb as compared to a parental Fc region, wherein the heterodimeric Fc variant is a variant of an immunoglobulin G (IgG) Fc, and wherein the numbering of amino acids is according to the EU index.

In another aspect, the present disclosure relates to a method of preparing a heterodimeric Fc variant having increased selectivity for a target receptor as compared to a parental Fc region, the heterodimeric Fc variant comprising a first Fc polypeptide and a second Fc polypeptide, the method comprising: (a) using an in silico model of the parental Fc region complexed with the target receptor: (i) inserting a sequence of one or more amino acid residues into a natural loop of one of the Fc polypeptides such that the natural loop is extended in length to provide a candidate variant, (ii) determining the distance of at least one of the amino acid residues of the inserted sequence from a target amino acid residue in the receptor, and (iii) selecting the candidate variant as the heterodimeric Fc variant if the at least one amino acid residue of the inserted sequence is within a heavy atom to heavy atom distance of 3 Å of the target amino acid residue in the receptor; (b) preparing nucleic acid encoding the heterodimeric Fc variant, and (c) expressing the nucleic acid in a host cell to provide the heterodimeric Fc variant, wherein the target receptor is FcγRIIb.

In another aspect, the present disclosure relates to a heterodimeric Fc variant comprising a first Fc polypeptide and a second Fc polypeptide, the heterodimeric Fc variant having increased selectivity of binding to FcγRIIb as compared to a parental Fc region, the heterodimeric Fc variant comprising an asymmetric mutation at position 236, wherein one of the Fc polypeptides comprises the mutation G236N or G236D, wherein the heterodimeric Fc variant is a variant of an immunoglobulin G (IgG) Fc, and wherein the numbering of amino acids is according to the EU index.

In another aspect, the present disclosure relates to a polypeptide comprising a heterodimeric Fc variant as disclosed herein, and one or more proteinaceous moieties fused or covalently attached to the heterodimeric Fc variant.

In another aspect, the present disclosure relates to a pharmaceutical composition comprising a heterodimeric Fc variant as disclosed herein or a polypeptide comprising the heterodimeric variant and one or more proteinaceous moieties, and a pharmaceutically acceptable carrier or diluent.

In another aspect, the present disclosure relates to a polypeptide comprising a heterodimeric Fc variant as disclosed herein and one or more proteinaceous moieties fused or covalently attached to the heterodimeric Fc variant, for use in therapy.

In another aspect, the present disclosure relates to a polypeptide comprising a heterodimeric Fc variant as disclosed herein and one or more proteinaceous moieties fused or covalently attached to the heterodimeric Fc variant, for use in the treatment of cancer, wherein at least one of the proteinaceous moieties is an antigen-binding domain that binds to a tumour-associated antigen or tumour-specific antigen.

In another aspect, the present disclosure relates to a method of treatment comprising administering to a patient in need thereof a polypeptide comprising a heterodimeric Fc variant and one or more proteinaceous moieties fused or covalently attached to the heterodimeric Fc variant.

In another aspect, the present disclosure relates to a method of treating cancer comprising administering to a patient in need thereof a polypeptide comprising a heterodimeric Fc variant and one or more proteinaceous moieties fused or covalently attached to the heterodimeric Fc variant, wherein at least one of the proteinaceous moieties is an antigen-binding domain that binds to a tumour-associated antigen or tumour-specific antigen

In another aspect, the present disclosure relates to a nucleic acid encoding a heterodimeric Fc variant as disclosed herein, or a polypeptide comprising a heterodimeric Fc variant and one or more proteinaceous moieties fused or covalently attached to the heterodimeric Fc variant. In another aspect, the present disclosure relates to a host cell comprising the nucleic acid.

In another aspect, the present disclosure relates to a method of preparing a heterodimeric Fc variant as disclosed herein, or a polypeptide comprising a heterodimeric Fc variant and one or more proteinaceous moieties fused or covalently attached to the heterodimeric Fc variant, the method comprising expressing nucleic acid encoding the heterodimeric Fc variant or polypeptide in a host cell.

### BRIEF DESCRIPTION OF THE DRAWINGS

FIG. 1 provides an overview of the steps taken to generate variants selective for FcγRIIb. LVG1=Lead Variants Generation 1; LVG2=Lead Variants Generation 2.

FIG. 2 shows the two approaches followed to introduce FcγRIIb selectivity into the Fc region: (A) introduction of asymmetric point mutations, and (B) asymmetric replacement of Loop 3.

FIG. 3 shows a cartoon representation of the in silico model built for IgG1 Fc bound to FcγRIIb.

FIG. 4 shows the sequence alignment between IgG1 and IgG4, showing the differences at positions 234, 268, 274, 296, 327 and 331 in the lower hinge and CH2 domain.

FIG. 5 shows a comparison of the crystal structures 1E4K and 1T83 of the Fc/FcγR complex showing the two possible binding modes by which the FcγR can bind the Fc region.

FIG. 6 shows a schematic representation of the method used to determine the contribution of a given mutation in each Fc chain to FcγR binding. The mutation G236A is used as an exemplary mutation and E269K is used as a polarity driver, which blocks binding to the FcγR only in the binding mode in which it is most proximal to position L135 (and R134) in the receptor. This binding mode is marked with a cross in FIG. 6.

FIG. 7 shows the parts of a generalized loop “template.” Loop templates are composed of N- and C-side β-stranded regions that extend the existing β-strands of the CH2 domain (shown in light grey), and an unstructured loop region (shown in dark grey). Templates were grafted into the CH2 domain by aligning the anchor residues of the template with residues B/324 and B/332 in the CH2 domain. The anchor residues are not grafted with the rest of the template.

FIG. 8 shows the length distribution of the loop templates identified in the initial search of the Protein Data Bank (PDB).

FIG. 9 shows a schematic representation of the structure of the human IgG1 Fc/FcγRIII complex available under the Protein Data Bank (PDB) ID 1E4K (Chain A (in green) is characterized by hotspot P329, and chain B (in cyan) is characterized by hotspot D270).

FIG. 10 shows (A) a summary of the improvement in affinity for FcγRIIb with respect to the wild-type (WT), and (B) a summary of the improvement in selectivity for FcγRIIb with respect to the wild-type (WT), for variants generated by Strategy 1 optimization of lead variant v19544. Positions 325-331B are within the inserted loop sequence and are otherwise referenced herein with an asterisk (i.e. 325*, 326*, etc.). The insets show heat maps of the positions showing the approximate location of positions 329 and 330 (329* and 330*) in the Fc relative to position S135 in FcγRIIb.

FIG. 11 shows (A) a summary of the improvement in affinity for FcγRIIb with respect to the wild-type (WT), and (B) a summary of the improvement in selectivity for FcγRIIb with respect to the wild-type (WT), for variants generated by Strategy 2 optimization of lead variant v19585.

FIG. 12 shows (A) a summary of the improvement in affinity for FcγRIIb with respect to the wild-type (WT), and (B) a summary of the improvement in selectivity for FcγRIIb with respect to the wild-type (WT), for variants generated by Strategy 3 (combination of lead variant v19544 with various loop replacements).

FIG. 13 shows (A) a summary of the improvement in affinity for FcγRIIb with respect to the wild-type (WT), and (B) a summary of the improvement in selectivity for FcγRIIb with respect to the wild-type (WT), for variants generated by Strategy 4 (combination of lead variant v19544 with longer loop replacements).

FIG. 14 shows a plot summarizing FcγRIIb binding and selectivity, C1q binding, change in FcγRIIb binding and aggregation propensity with pH, and change in Tm for variants v32210, v32226, v32295, v32230, v32227, v32274 and v32284.

FIG. 15 shows the correlation between CDC activity and C1q binding using a Spearman Rank test (R=0.94, p<1e-12) for anti-CD40 antibodies comprising variants v22096, v26370, v26774, v27092, v31186, v31188, v31191, v31192, v31213, v32210, v32211, v32212, v32226, v32227, v32230, v32231, v32242, v32274, v32282, v32284, v32287, v32288, v32292, v32293, v32294, v32295 and v32296, as well as controls (wild-type, negative, v12 and SELF).

FIG. 16 shows the serum human C5 antigen levels in human FcγR2b transgenic mice following 1 mg/kg dosing of anti-C5 antibodies with differing affinities to human FcγRIIb. Treatment groups consisted of n=5 (Neg, v31188 and v32227), n=4 (v21653 (WT) and v32284) and n=2 (no Ab group). Values shown are mean±SEM.

FIG. 17 shows the serum antibody concentration in human FcγR2b transgenic mice following 1 mg/kg dosing of anti-C5 antibodies with differing affinities to human FcγRIIb. Treatment groups consisted of n=5 (Neg, v31188 and v32227) and n=4 (v21653 (WT) and v32284). Results from one animal in each of the v32227 and v32284 groups was omitted as profiles resemble SC/IP rather than IV dosing. Values shown are mean±SEM.

### DETAILED DESCRIPTION

Described herein are heterodimeric Fc variants comprising one or more asymmetric amino acid mutations in the CH2 domain and having increased selectivity of binding to FcγRIIb as compared to a parental Fc region. In some embodiments, the heterodimeric Fc variants described herein have increased selectivity of binding to FcγRIIb and increased binding affinity for FcγRIIb as compared to the parental Fc region. A “parental Fc region” is an Fc region that is identical to the heterodimeric Fc variant except that it lacks the one or more amino acid mutations in the CH2 domain that increase binding selectivity and/or affinity for FcγRIIb. The one or more asymmetric mutations comprise replacement of a loop in the CH2 domain, a mutation at position 236 in the CH2 domain, or a combination of replacement of a loop in the CH2 domain and a mutation at position 236 in the CH2 domain.

Certain embodiments of the present disclosure relate to polypeptides comprising a heterodimeric Fc variant as described herein. Examples of such polypeptides include, but are not limited to, antibodies, antibody fragments and Fc fusion proteins. Polypeptides comprising a heterodimeric Fc variant may find use as therapeutics, diagnostics or research tools.

Certain embodiments of the present disclosure relate to polynucleotides encoding the heterodimeric Fc variants and polynucleotides encoding the polypeptides comprising the heterodimeric Fc variants, as well as host cells comprising the polynucleotides and methods of using the polynucleotides and host cells to prepare the heterodimeric Fc variants or polypeptides comprising the heterodimeric Fc variants.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to, unless clearly indicated otherwise.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a Fc variant, composition, use or method, denotes that additional elements and/or method steps may be present, but that these additions do not materially affect the manner in which the recited Fc variant, composition, method or use functions. The term “consisting of” when used herein in connection with a Fc variant, composition, use or method, excludes the presence of additional elements and/or method steps. A Fc variant, composition, use or method described herein as comprising certain elements and/or steps may also, in certain embodiments consist essentially of those elements and/or steps, and in other embodiments consist of those elements and/or steps, whether or not these embodiments are specifically referred to.

The term “derived from” when used herein to describe an amino acid sequence, means that the subject amino acid sequence is substantially identical to a reference amino acid sequence from which it is derived.

By “substantially identical” as used herein in connection with an amino acid sequence, it is meant that, when optimally aligned (for example using the methods described below), the amino acid sequence shares at least 50%, at least 60%, at least 70%, at least 75%, at least 80%, at least 85% or at least 90% sequence identity with its reference amino acid sequence. Percent identity between two amino acid sequences may be determined in various ways known in the art, for example, using publicly available computer software such as Smith Waterman Alignment (Smith & Waterman, 1981, J Mol Biol 147:195-7); “BestFit” (Smith & Waterman, 1981, Advances in Applied Mathematics, 482-489); BLAST (Basic Local Alignment Search Tool; (Altschul, et al., 1990, J Mol Biol, 215:403-10) and variations and updates thereof, ALIGN, ALIGN-2, CLUSTAL or Megalign (DNASTAR) software. In addition, those skilled in the art can determine appropriate parameters for measuring alignment, including algorithms needed to achieve maximal alignment over the length of the sequences being compared. In general, for peptides, the length of comparison sequences will be at least 10 amino acids, but one skilled in the art will understand that the actual length will depend on the overall length of the sequences being compared. In certain embodiments, the length of comparison sequences may be the full-length of the peptide or polypeptide sequence.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The terms “Fc region” and “Fc,” as used interchangeably herein, refer to a C-terminal region of an immunoglobulin heavy chain. Although the boundaries of the Fc region of an immunoglobulin heavy chain may vary, the human IgG heavy chain Fc region sequence, for example, is usually defined as extending from position 239 to the C-terminus of the heavy chain. An “Fc polypeptide” of a dimeric Fc refers to one of the two polypeptides forming the dimeric Fc domain, i.e. a polypeptide comprising C-terminal constant regions of an immunoglobulin heavy chain that is capable of stable self-association. An Fc region typically comprises a CH2 domain and a CH3 domain. The Fc region may also be considered to encompass the hinge region in certain embodiments.

The “CH2 domain” of a human IgG Fc region is typically defined as extending from position 239 to position 340. The “CH3 domain” is typically defined as comprising the amino acids residues C-terminal to the CH2 domain in an Fc region, i.e. from position 341 to position 447. The “hinge region” of human IgG1 is generally defined as extending from position 216 to position 238 (Burton, 1985, Molec. Immunol., 22:161-206). Hinge regions of other IgG isotypes may be aligned with the IgG1 sequence by aligning the first and last cysteine residues that form inter-heavy chain disulfide bonds.

Unless otherwise specified herein, numbering of amino acid residues in the Fc region is according to the EU numbering system, also called the EU index, as described in Kabat et al, Sequences of Proteins of Immunological Interest, 5th Ed. Public Health Service, National Institutes of Health, Bethesda, MD (1991).

It is to be understood that the positive recitation of a feature in one embodiment, serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

It is contemplated that any embodiment discussed herein can be implemented with respect to an Fc variant, method, use or composition disclosed herein, and vice versa.

### Heterodimeric Fc Variants

The heterodimeric Fc variants of the present disclosure comprise one or more asymmetric amino acid mutations in the CH2 domain and have increased selectivity of binding to FcγRIIb as compared to the parental Fc region. In some embodiments, the heterodimeric Fc variants also have increased binding affinity for FcγRIIb as compared to the parental Fc region.

Increased selectivity of binding to FcγRIIb, also referred to herein as “increased selectivity for FcγRIIb,” means that the heterodimeric Fc variant shows a greater binding affinity for FcγRIIb relative to its binding affinity for the other Fcγ receptors, and in particular relative to its binding affinity for FcγRIIaR, as compared to the parental Fc region. In certain embodiments, the increased selectivity of the heterodimeric Fc region for FcγRIIb is defined relative to its binding affinity for FcγRIIaR. In certain embodiments as described herein, the increased selectivity of a heterodimeric Fc variant for FcγRIIb relative to FcγRIIaR may be expressed as the fold increase over the FcγRIIb selectivity of the parental Fc region. For example, in some embodiments, a heterodimeric Fc variant may have a selectivity for FcγRIIb that is increased by at least 1.5-fold over the parental Fc region, or at least 2-fold over the parental Fc region.

An increase in FcγRIIb selectivity may or may not be accompanied by an increase in FcγRIIb affinity as compared to the parental Fc region. Accordingly, in certain embodiments, a heterodimeric Fc variant may have an increased selectivity for FcγRIIb as compared to the parental Fc region, for example an increase in FcγRIIb selectivity of at least 1.5-fold over the parental Fc region, but no increase in FcγRIIb affinity. In certain embodiments, a heterodimeric Fc variant may have an increased selectivity for FcγRIIb as compared to the parental Fc region, for example an increase in FcγRIIb selectivity of at least 1.5-fold over the parental Fc region, and a decrease in FcγRIIb affinity as compared to the parental Fc region.

In certain embodiments, a heterodimeric Fc variant may have an increased selectivity for FcγRIIb as compared to the parental Fc region, for example an increase in FcγRIIb selectivity of at least 1.5-fold over the parental Fc region, and substantially the same FcγRIIb affinity as compared to the parental Fc region. In certain embodiments, a heterodimeric Fc variant may have an increased selectivity for FcγRIIb as compared to the parental Fc region, for example an increase in FcγRIIb selectivity of at least 1.5-fold over the parental Fc region, and also an increase in FcγRIIb affinity as compared to the parental Fc region.

Increased binding affinity for FcγRIIb, also referred to herein as “increased affinity for FcγRIIb,” means that the heterodimeric Fc variant shows an increased binding affinity for FcγRIIb as compared to the binding affinity of the parental Fc for FcγRIIb. In certain embodiments as described herein, the increased affinity of a heterodimeric Fc variant for FcγRIIb may be expressed as the fold increase over the affinity of the parental Fc region for FcγRIIb. For example, in some embodiments, a heterodimeric Fc variant may have an affinity for FcγRIIb that is increased by at least 10-fold over the parental Fc region.

The heterodimeric Fc variants comprise two heavy chain constant domain polypeptides, referred to herein as a first Fc polypeptide and a second Fc polypeptide. It is to be understood that the designation “first” and “second” with respect to the Fc polypeptides is for convenience only and that the two Fc polypeptides are interchangeable provided that the Fc variant comprises one first Fc polypeptide and one second Fc polypeptide.

An “asymmetric” amino acid mutation in the context of the present disclosure means that one Fc polypeptide comprises an amino acid mutation at a specified position and the other Fc polypeptide either does not comprise an amino acid mutation at the corresponding position or comprises a different amino acid mutation at the corresponding position. The first and second Fc polypeptides of a heterodimeric Fc variant may comprise one or more than one asymmetric amino acid mutation. The amino acid mutation may be a substitution, insertion or deletion of an amino acid, or replacement of a sequence of one or more amino acids with an alternative sequence. The alternative sequence may be the same length as the sequence it is replacing (i.e. comprise the same number of amino acids) or it may be longer than the sequence that it is replacing (i.e. comprise additional amino acids). In certain embodiments, the one or more asymmetric amino acid mutations comprised by the heterodimeric Fc variant comprise substitutions of one or more amino acids. In some embodiments, the one or more asymmetric amino acid mutations comprised by the heterodimeric Fc variants comprise an asymmetric loop replacement in which a loop sequence in the CH2 domain of one Fc polypeptide is replaced by a different polypeptide loop sequence. In some embodiments, the one or more asymmetric amino acid mutations comprised by the heterodimeric Fc variants comprise substitutions of one or more amino acids and an asymmetric loop replacement in which a loop sequence in the CH2 domain of one Fc polypeptide is replaced by a different polypeptide loop sequence.

In certain embodiments, the one or more asymmetric amino acid mutations comprised by the heterodimeric Fc variant comprise an asymmetric loop replacement in the CH2 domain, a mutation at position 236, or a combination of an asymmetric loop replacement in the CH2 domain and a mutation at position 236. When the heterodimeric Fc variant comprises an asymmetric loop replacement in the CH2 domain and a mutation at position 236, the mutation at position 236 may be a symmetric mutation or an asymmetric mutation. In some embodiments, the heterodimeric Fc variant comprises an asymmetric loop replacement in the CH2 domain and a symmetric mutation at position 236. In some embodiments, the heterodimeric Fc variant comprises an asymmetric loop replacement in the CH2 domain and an asymmetric mutation at position 236.

In certain embodiments, the heterodimeric Fc variant comprises an asymmetric loop replacement in the CH2 domain. In some embodiments, the heterodimeric Fc variant comprises an asymmetric loop replacement in the CH2 domain and one or more additional amino acid mutations in the CH2 domain. The one or more additional amino acid mutations may be asymmetric or symmetric mutations.

In certain embodiments, the heterodimeric Fc variant comprises an asymmetric mutation at position 236. In some embodiments, the heterodimeric Fc variant comprises an asymmetric mutation at position 236 and one or more additional amino acid mutations in the CH2 domain. The one or more additional amino acid mutations may be asymmetric or symmetric mutations.

Examples of heterodimeric Fc variants include, but are not limited to, heterodimeric Fc variants comprising the amino acid mutations as set out for any one of the variants shown in Table 5A, Table 5B, Table 5C, Table 13.1, Table 6.22, Table 6.23, Table 6.24, Table 6.25, Table 6.26 and Table 6.27. Additional heterodimeric Fc variants are described below.

In certain embodiments, the heterodimeric Fc variant comprises the amino acid mutations as set out for any one of the variants shown in Table 5A, Table 5B, Table 5C, Table 13.1, Table 6.22, Table 6.23 and Table 6.24. In some embodiments, the heterodimeric Fc variant comprises the amino acid mutations as set out for any one of the variants shown in Table 5A, Table 5B, Table 5C and Table 13.1.

When the heterodimeric Fc variant comprises more than one amino acid mutation, each individual mutation comprised by the heterodimeric Fc variant may result in an increase in selectivity of the heterodimeric Fc variant for FcγRIIb, an increase in affinity of the heterodimeric Fc variant for FcγRIIb, or an increase in both selectivity and affinity of the heterodimeric Fc variant for FcγRIIb, but taken together the amino acid mutations result in a heterodimeric Fc variant having increased selectivity for FcγRIIb, and optionally increased affinity for FcγRIIb. Thus, in certain embodiments, the amino acid mutations comprised by the heterodimeric Fc variant may comprise one or more amino acid mutations that result in an increase in selectivity of the heterodimeric Fc variant for FcγRIIb and optionally one or more different amino acid mutations that result in an increase in affinity for FcγRIIb. In some embodiments, the one or more amino acid mutations comprised by the heterodimeric Fc result in an increase in selectivity of the heterodimeric Fc variant for FcγRIIb and an increase in affinity for FcγRIIb.

When the heterodimeric Fc variants described herein comprise more than one amino acid mutation the increases the selectivity and/or affinity for FcγRIIb, the heterodimeric Fc variant may comprise up to 20 such amino acid mutations in total, where an asymmetric loop insertion is considered to be one amino acid mutation. In certain embodiments, the heterodimeric Fc variant comprises between 1 and 20 amino acid mutations, where an asymmetric loop insertion is considered to be one amino acid mutation. In certain embodiments, the heterodimeric Fc variant comprises between 1 and 18 amino acid mutations, between 1 and 16 amino acid mutations or between 1 and 15 amino acid mutations, where an asymmetric loop insertion is considered to be one amino acid mutation.

In certain embodiments, the heterodimeric Fc variant is a variant of an immunoglobulin G (IgG) Fc. In some embodiments, the heterodimeric Fc variant is a variant of a human IgG Fc. In some embodiments, the heterodimeric Fc variant is a variant of an IgG1 Fc. In some embodiments, the heterodimeric Fc variant is a variant of a human IgG1 Fc. The amino acid sequence of the native human IgG1 Fc from position 231 to 447 is provided in Table 1 (SEQ ID NO:1).

TABLE 1
Human IgG1 Fc Sequence

| | |
|---|---|
| Human IgG1 Fc | APELLGGPSVFLFPPKPKDTLMISRTPEVT |
| sequence 231-447 | CVVVDVSHEDPEVKFNWYVDGVEVHNAKTK |
| (EU-numbering) | PREEQYNSTYRVVSVLTVLHQDWLNGKEYK |
| | CKVSNKALPAPIEKTISKAKGQPREPQVYT |
| | LPPSRDELTKNQVSLTCLVKGFYPSDIAVE |
| | WESNGQPENNYKTTPPVLDSDGSFFLYSKL |
| | TVDKSRWQQGNVFSCSVMHEALHNHYTQKS |
| | LSLSPGK (SEQ ID NO: 1) |

### Heterodimeric Fc Variants Comprising an Asymmetric Loop Replacement in the CH2 Domain

Certain embodiments of the present disclosure relate to heterodimeric Fc variants having increased selectivity for FcγRIIb as compared to a parental Fc region, in which one of the Fc polypeptides of the heterodimeric Fc variant comprises replacement of all or a part of a natural loop in the CH2 domain of the Fc polypeptide with an alternative amino acid sequence such that the natural loop is extended in length and the affinity of the heterodimeric variant for FcγRIIb is increased. Some embodiments relate to methods of designing such heterodimeric Fc variants.

Accordingly, certain embodiments of the present disclosure relate to a method for designing a heterodimeric Fc variant having increased selectivity for a target receptor as compared to a parental Fc region, the method comprising: (i) in an in silico model of the parental Fc region complexed with the target receptor, replacing all or a part of a natural loop sequence in the CH2 domain of one of the Fc polypeptides of the Fc variant with an alternative amino acid sequence such that the natural loop is extended in length to provide a candidate variant; (ii) determining the distance of at least one of the amino acid residues of the alternative amino acid sequence from a target amino acid residue in the receptor, and (iii) selecting the candidate variant as the heterodimeric Fc variant if the at least one amino acid residue of the alternative amino acid sequence is within a heavy atom to heavy atom distance of 3 Å of the target amino acid residue in the receptor. In certain embodiments, the target receptor is FcγRIIb.

In some embodiments, the method further comprises: preparing nucleic acid encoding the heterodimeric Fc variant, and expressing the nucleic acid in a host cell to provide the heterodimeric Fc variant.

Certain embodiments of the present disclosure relate to heterodimeric Fc variants having increased selectivity for FcγRIIb as compared to a parental Fc region, in which one of the Fc polypeptides of the heterodimeric Fc variant comprises replacement of all or a part of a natural loop in the CH2 domain of the Fc polypeptide with an alternative amino acid sequence such that the loop is extended in length and interactions between the Fc polypeptide and the receptor are increased. For example, the replacement loop may modify the interactions between one or more other loops in the Fc polypeptide and the receptor such that binding of the Fc polypeptide to the receptor is improved, or at least one of the residues of the replacement loop may be in close proximity to a target amino acid in the receptor such that interactions between the Fc polypeptide and receptor are increased. In certain embodiments, at least one of the amino acid residues of the replacement loop is within a heavy atom to heavy atom distance of 3 Å of a target amino acid residue in the receptor when the heterodimeric Fc variant is bound by the receptor. In certain embodiments, the target amino acid residue in the receptor is Ser 135.

In some embodiments, the replacement loop sequence is a polypeptide between 7 and 15 amino acids in length or between 8 and 15 amino acids in length. In some embodiments, the natural loop comprises amino acids 325 to 331 of the Fc polypeptide.

The terms “replacement loop,” “replacement loop sequence” and “loop replacement” are used interchangeably herein with reference to the sequence used to replace all or a part of the selected natural loop in the CH2 domain of the heterodimeric Fc polypeptide. Similarly, the terms “polypeptide” and “polypeptide loop” are used interchangeably when describing the replacement loop sequence.

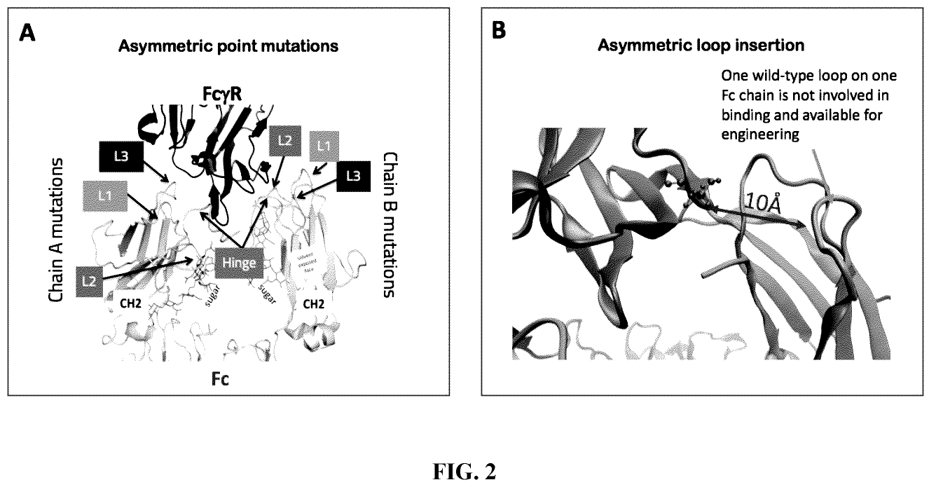

As described herein, the loop at positions 325 to 331 in the CH2 domain of one of the Fc polypeptides of the IgG Fc is not directly involved in FcγR binding as the residues comprised by this loop are typically distant from position 135 on the FcγR (see FIG. 2B). The loop at positions 325 to 331 of the IgG1 CH2 domain is sometimes referred to as the “FG Loop” or “Loop 3.” As also described herein, replacing the FG loop of one of the Fc polypeptides with a polypeptide loop engineered to interact with FcγRIIb near residue 135 improves selective binding of the Fc to the receptor. In certain embodiments, the heterodimeric Fc variant of the present disclosure comprises an asymmetric replacement of the FG loop and has increased selectivity for FcγRIIb as compared to the parental Fc. In some embodiments, the heterodimeric Fc variant comprises an asymmetric replacement of the FG loop and optionally one or more additional amino acid mutations in the CH2 domain and has increased selectivity for FcγRIIb as compared to the parental Fc. In some embodiments, the heterodimeric Fc variant comprises an asymmetric replacement of the FG loop and optionally one or more additional amino acid mutations in the CH2 domain and has increased selectivity for FcγRIIb and increased affinity for FcγRIIb as compared to the parental Fc. The one or more additional amino acid mutations may be asymmetric or symmetric mutations. In certain embodiments, the one or more additional amino acid mutations comprise a mutation at position 236 in one or both of the Fc polypeptides.

### Asymmetric Loop Replacement

In certain embodiments, the heterodimeric Fc variant comprises an asymmetric loop replacement in the CH2 domain and has increased selectivity for FcγRIIb as compared to the parental Fc. In some embodiments, the asymmetric loop replacement comprised by the heterodimeric Fc variant comprises replacement of the native loop at positions 325 to 331 in one Fc polypeptide with a polypeptide loop of between 7 and 15 amino acids in length, for example, between 7 and 12 amino acids in length. In some embodiments, the asymmetric loop replacement comprised by the heterodimeric Fc variant comprises replacement of the native loop at positions 325 to 331 in one Fc polypeptide with a longer polypeptide loop, for example, a polypeptide loop of between 8 and 15 amino acids in length, between 8 and 14 amino acids in length, or between 8 and 12 amino acids in length. In some embodiments, the asymmetric loop replacement comprised by the heterodimeric Fc variant comprises replacement of the native loop at positions 325 to 331 in one Fc polypeptide with a polypeptide loop of between 9 and 15 amino acids in length, between 9 and 14 amino acids in length, between 10 and 15 amino acids in length or between 10 and 14 amino acids in length.

In some embodiments, the polypeptide loop that replaces the native loop in the Fc variant is derived from the sequence of a loop-forming segment of a second protein. Identification of suitable loop-forming segments of known proteins may be achieved using methods such as those described herein (see Example 2). For example, candidate loop sequences may be identified by analyzing the structures of known proteins, such as those structures available through the Protein Data Bank (PDB) (Berman, et al., 2000, Nucl. Acids Res., 28:235-242). The PDB is accessible, for example, via the website maintained by the Research Collaboratory for Structural Bioinformatics (RCSB). To facilitate identification of candidate loop sequences, the protein structures selected for analysis may be limited to those having crystal structures with a specified level of resolution, for example, a resolution of 2.5 Å or higher.

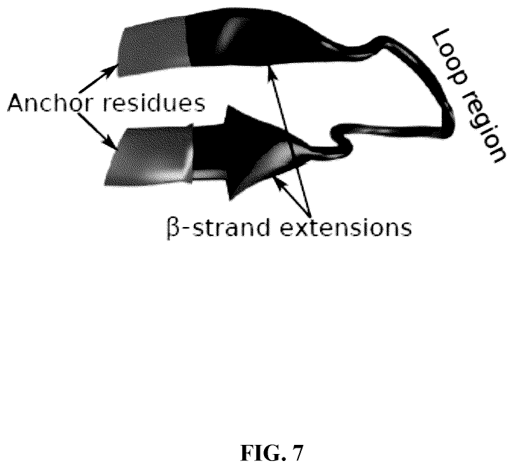

Candidate loop sequences (“templates”) are typically loop sequences that are anchored in their parent protein by β-strands. The general structure of a suitable loop sequence is shown in FIG. 7. In this general structure, the loop template is composed of an unstructured loop region and N-terminal and C-terminal β-stranded regions, which can function to extend the existing β-strands that are present in the Fc CH2 domain. The anchor residues of the template allow for alignment with the amino acids present at positions 324 and 332 in the CH2 domain, but the anchor residues do not form part of the template.

Once candidate loop sequences have been identified, secondary structure may be assigned to the amino acids of the selected PDB protein structures using one or a combination of various algorithms known in the art, such as STRIDE (Frishman & Argos, 1995, Proteins Struct. Funct. Bioinf, 23:566-579), DSSP (Kabsch & Sander, 1983, Biopolymers, 22:2577-2637), DEFINE (Richards & Kundrot, 1988, Proteins, 3:71-84), ScrewFit (Calligari & Kneller, 2012, Acta Crystallographica Section D. 68: 1690-3) or SST (Konagurthu et al., 2012, Bioinformatics, 28:i97-i105).

In some embodiments, candidate polypeptide loops may be identified from PDB protein structures using the following selection criteria:
i) the loop sequence is anchored in the parent protein by beta strands;
ii) the loop sequence includes one or more beta-stranded amino acids at each of the loop N-terminus and C-terminus;
iii) the one or more beta-stranded amino acids at the C-terminus of the polypeptide loop do not form hydrogen bonds with any amino acid in the parent protein except the beta-stranded amino acids at the N-terminus of the polypeptide loop, and
iv) the backbone heavy atom root mean square deviation (RMSD) of the one or more beta-stranded amino acids at each of the N-terminus and C-terminus of polypeptide loop to one or more amino acids ending at site 324 (for the N-terminus) and beginning at site 332 (for the C-terminus) in the CH2 domain is ≤0:85 Å.

In some embodiments, the following additional criterion may be used to identify candidate polypeptide loops:
v) the loop sequence includes at least one hydrogen bond between beta-stranded amino acids at opposite termini of the polypeptide loop.

Once candidate polypeptide loops have been identified, they may be further analysed in order to select appropriate templates for use to replace the native loop in the Fc variant.

In certain embodiments, the candidate polypeptide loops may be grafted in silico into an Fc/FcγRIIb complex for further analysis. In some embodiments, the in silico grafting may comprise the following steps:
i) delete residues 325-331 inclusive from the Fc/FcγRIIb complex;
ii) introduce the template backbone into the Fc/FcγRIIb complex by aligning the backbone heavy atoms of the template anchors to residues 324 and 332 of the Fc/FcγRIIb complex, and
iii) minimize the coordinates of the backbone atoms for residues 323, 324, 332, 333 and the first two and last two residues of the template.

Step iii) above may be achieved using conventional software, for example, the AMBER99SB force field (Hornak, et al., 2006, Proteins Struc. Funct. Bioinf, 65:712) and a conjugate gradient minimizer.

The grafted candidate polypeptide loops may then be further screened by applying a filter to identify those templates that, in their grafted configuration, have a length and orientation that may permit one or more template residues to interact with FcγRIIb at or near position 135 on the FcγR. For example, a coarse contact potential filter may be applied to the grafted candidate polypeptide loops. In the Examples provided herein, the following coarse contact potential was developed and may be used for this purpose:

$$c ( {\vec{r}}_{i} , {\vec{r}}_{j} ) = \{ \begin{matrix}1 & if {d}_{ij} < ‖ {\vec{r}}_{i} - {\vec{r}}_{j} \leq α ( i , j ) \\ 0 & otherwise\end{matrix} \qquad [ 1 ]$$

where dᵢⱼ is the sum of the van der Waals radii for atoms i and j (rᵢ and rⱼ, respectively), and the empirical upper bound on the contact distance between two atoms is defined as:

$$α ( i , j ) = \{ \begin{matrix}9 A & if atoms i , j are both {C}_{β} atoms \\ 7.5 A & if one of atoms i , j is a {C}_{β} atoms \\ 6 A & otherwise\end{matrix} \qquad [ 2 ]$$

and where c (i;j) is computed between C_{β} and backbone heavy atoms of residues comprised by the template, and the C_{β} and backbone heavy atoms of residue 135 on the FcγR.

In applying the above coarse contact potential filter, a minimum coarse contact count of between 5 and 10 may be used. For example, a minimum coarse contact count of 6, 7 or 8 may be used.

Candidate polypeptide loops that pass the coarse contact filter may then undergo structure optimization. This step comprises side-chain repacking with backbone relaxation. The side-chain repacking procedure employed in the Examples provided herein is a variant of the ICM algorithm with a fine-grained rotamer library (see Xiang & Honig, 2001, J. Mol. Biol., 311:421), and backbone coordinates were relaxed via 5000 steps of the backrub algorithm (see Betancourt, 2005, J Chem. Phys., 123:174905; Smith & Kortemme, 2008, J. Mol. Biol., 380:742). When repacking, the sequence of the candidate polypeptide loop was taken to be the wild-type sequence as found in the PDB structure from which the polypeptide loop sequence was taken.

The above steps may be performed, for example, using the AMBER99SB force-field (Hornak, et al., 2006, Proteins Struc. Funct. Bioinf, 65:712), the GB/OBC implicit solvent model (Onufriev, et al., 2004, Proteins Struc. Funct. Bioinf, 55:383), and a pairwise hydrophobic potential (Jacobsen, et al., 2004, Proteins Struc. Funct. Bioinf, 55:351).

After repacking and backbone optimization, the grafted candidate polypeptide loops may be checked for inter-atomic clashes. In certain embodiments, atoms i and j are considered to be clashing when σᵢ+σⱼ−dᵢⱼ>0.4, where σᵢ is the van der Waals radius of atom i as defined in the AMBER99SB force field, and dᵢⱼ is the distance between atoms i and j. Candidate polypeptide loops that do not show inter-atomic clashes after repacking are selected for further analysis and may be re-evaluated using the coarse contact score. The minimum C_{β}-C_{β} distance between any residue on the polypeptide loop and the C_{β} atom on receptor residue 135 is also computed.

The Pareto Optimal templates are then identified on the basis of anchor backbone heavy atom RMSD, coarse contact score and minimum C_{β}-C_{β} distance. The Pareto Optimal Consensus (POC) method (Li, et al., 2010, BMC Struc. Biol., 10:22) is a consensus model ranking approach to integrate multiple knowledge- or physics-based scoring functions. The procedure of identifying the models of best quality in a model set includes: 1) identifying the models at the Pareto optimal front with respect to a set of scoring functions, and 2) ranking them based on the fuzzy dominance relationship to the rest of the models.

For the candidate polypeptide loops, those loops on the first three Pareto optimal fronts are identified and pairwise sequence similarities computed for all candidate polypeptide loops of a common length in the optimal set.

As a next step, the stability of the template conformations in the Fc/FcγRIIb complex is tested using a simple implicit water molecular dynamics-based simulated annealing approach. This step is undertaken to account for a change in conformation of the candidate polypeptide loops in the new Fc/FcγR complex environment, which is assumed to be different to the native environment of the loops.

For the molecular-dynamics based simulated annealing approach, a mobile region is first defined by placing an arginine residue at each site on the candidate polypeptide loop, rotating the residue through every rotamer in the Dunbrack rotamer library (Dunbrack & Karplus, 1993, J. Mol. Biol., 230:543) and enumerating all Fc/FcγR residues with a heavy atom less than 4.0 Å from a heavy atom of the test arginine in any rotameric configuration. The union of all residues identified in this manner results in a “mobile zone.” All residues not included in the mobile zone are held fixed, whereas residues within this zone are unrestricted. Once the mobile zone is defined for a candidate polypeptide loop, the loop is run through a simulated annealing protocol using, for example, the OpenMM molecular dynamics package (Eastman, et al., 2013, J. Chem. Theory Comput., 9:461), the AMBER99SB force-field and the GB/OBC implicit solvation model.

An exemplary annealing protocol includes the following steps:
1. Performing a short (2 ns) high-temperature simulation at 500K.
2. Clustering the conformations from the second half of the trajectory produced in step 1 into ten clusters using the k-means algorithm.
3. Performing ten separate annealing simulations starting from the conformations identified in step 2. A sample temperature schedule comprises cooling geometrically from 500K to 450K over 1.0 ns, followed by a linear cooling stage from 450K to 300K over 19 ns.
4. Extracting the low temperature components (300K-302K) of each of the ten annealing trajectories for subsequent analysis. Combined, the ten annealing runs generate 3 ns of trajectory data for each candidate polypeptide loop.

The aggregate trajectory produced in step 4 of the annealing procedure is then clustered. Clustering is performed on the backbone heavy atoms of the template using, for example, the SPICKER clustering method (Zhang & Skolnick, 2004, J. Comput. Chem., 25:865). As the majority of the Fc/FcγR structure was held fixed during the annealing simulations, the variations in the conformations of templates will have contributions both from internal deformation of the template and relaxation of the anchoring β-strands. Only the primary cluster returned by the SPICKER algorithm is considered in further analysis.

By construction, the primary clusters contain between 60% and 70% of the total frames in the aggregate trajectory produced in step 4 of the annealing procedure. Using the primary clusters, the following quantities are computed:
1. The mean number of coarse contacts between the candidate polypeptide loop and residue 135 on the FcγRIIb receptor.
2. The root mean square fluctuations (RMSF) of the template (computed on the basis of the template backbone heavy atoms).
3. The mean backbone heavy atom root mean square deviation (RMSD) (computed relative to the grafted structure of the candidate polypeptide loop).

The coarse contact score provides an indication of whether the low-temperature structures generated by the annealing processes have configurations that are in position to interact with residue 135 in the FcγRIIb.

The RMSF serves as a measure of consistency between and within the annealing runs. A low RMSF value indicates that a candidate polypeptide loop shows consistency in structure across the annealing runs, which in turn indicates that the runs were well converged. A low RMSF value also indicates that a candidate polypeptide loop is not overly flexible. As such, candidate polypeptide loops with low RMSF are favoured for subsequent selection rounds.

A low backbone RMSD to the grafted structure indicates that a candidate polypeptide loop does not deviate significantly from the wildtype conformation found in the native PDB structure. Accordingly, candidate polypeptide loops that show a low backbone RMSD to the grafted conformation are also favoured.

The above set of metrics may be used to select a set of candidate polypeptide loops for experimental screening. In certain embodiments, the above set of metrics may be used to select candidate polypeptide loops using the following values: (a) a coarse contact count ≥5 and a reference RMSD less than 3.0 Å, or (b) a coarse contact count ≥5 and a RMSF less than 3.0 Å. In some embodiments, the above set of metrics may be used to select candidate polypeptide loops using the following values: (a) a coarse contact count ≥3 and a reference RMSD less than 1.5 Å, or (b) a coarse contact count ≥3 and a RMSF less than 1.5 Å.

Candidate polypeptide loops selected by the above approach may be tested experimentally by engineering a test antibody using standard molecular biology techniques to replace residues 325 to 331 in one Fc polypeptide of the test antibody with the candidate loop sequence, then testing the resulting variant antibody for FcγR binding using standard protocols such as those described herein. If necessary or desirable, one or more amino acid substitutions may be made to the loop sequence in order to increase selectivity or affinity of the variant antibody for FcγRIIb as described in the Examples provided herein.

Examples of candidate polypeptide loops identified using the approach outlined above are shown in Table 2.

TABLE 2
Examples of Candidate Polypeptide Loop Sequences

| | | | Source | Start | | | |
|---|---|---|---|---|---|---|---|
| SEQ ID | Template | | PDB | Residue | | RMSF | Coarse |
| NO | ID | Sequence | ID | ID | RMSD_{Ref}¹ | (Å) | Contacts |
| 4 | 231 | WTDQSGQDR | IQVC | 88.TRP | 1.81 ± 0.21 | 0.73 | 4 |
| 5 | 168 | LDMEGRKIH | 1LN1 | 123.LEU | 0.87 ± 0.09 | 0.33 | 5 |
| 6 | 1 | STWFDGGYAT | 2GKO | 235.SER | 1.94 ± 0.42 | 1.24 | 3 |
| 7 | 11 | HFDENGEIVT | 2DWC | 218.HIS | 0.77 ± 0.22 | 0.6 | 3 |
| 8 | 7 | GLDEEGKGAV | 4R3O | 112.GLY | 0.52 ± 0.12 | 0.43 | 16 |
| 9 | 19 | VTWEDGKSER | 1OID | 323.VAL | 0.90 ± 0.10 | 0.43 | 20 |
| 10 | 38 | LIDENGNEQK | 3GVE | 150.LEU | 0.81 ± 0.12 | 0.38 | 13 |
| 11 | 60 | VQDATGAPFL | 3E35 | 99.VAL | 1.05 ± 0.18 | 0.48 | 12 |
| 12 | 66 | DFDQNQGEVV | 1IJR | 47.ASP | 0.84 ± 0.18 | 0.53 | 23 |
| 13 | 83 | SDFEGKPTL | 2X6C | 151.SER | 0.88 ± 0.20 | 0.43 | 12 |
| 14 | 151 | LTDEEGRPYR | 4JN3 | 67.LEU | 0.84 ± 0.23 | 0.54 | 12 |

¹Averaged over the dominant cluster (obtained using SPIKER clustering)

In certain embodiments, the replacement loop comprised by the heterodimeric Fc variant is a polypeptide loop comprising an amino acid sequence that is substantially identical to a sequence as set forth in any one of SEQ ID NOs: 4, 5, 6, 7, 8, 9, 10, 11, 12, 13 or 14. In some embodiments, the polypeptide loop comprises an amino acid sequence that is a variant of the sequence as set forth in any one of SEQ ID NOs: 4, 5, 6, 7, 8, 9, 10, 11, 12, 13 or 14, where the variant comprises 1, 2, 3, 4 or 5 amino acid mutations. In some embodiments, the variant comprises 1, 2, 3 or 4 amino acid mutations. In some embodiments, the polypeptide loop comprises an amino acid sequence as set forth in any one of SEQ ID NOs: 4, 5, 6, 7, 8, 9, 10, 11, 12, 13 or 14.

In certain embodiments, the replacement loop comprised by the heterodimeric Fc variant is a polypeptide loop comprising an amino acid sequence that is substantially identical to a sequence as set forth in any one of SEQ ID NOs: 6, 8, 9, 12 or 14. In some embodiments, the polypeptide loop comprises an amino acid sequence that is a variant of the sequence as set forth in any one of SEQ ID NOs: 6, 8, 9, 12 or 14, where the variant comprises 1, 2, 3, 4 or 5 amino acid mutations. In some embodiments, the variant comprises 1, 2, 3 or 4 amino acid mutations. In some embodiments, the polypeptide loop comprises an amino acid sequence as set forth in any one of SEQ ID NOs: 6, 8, 9, 12 or 14.

In certain embodiments, the replacement loop comprised by the heterodimeric Fc variant is a polypeptide loop comprising an amino acid sequence as set forth in any one of Formula (I), Formula (Ia), Formula (Ib), Formula (II), Formula (ITT), Formula (IV), Formula (V) or Formula (VI), as shown below, where Formulae (I), (Ia) and (Ib) are derived from the sequence set forth in SEQ ID NO: 6, Formulae (II) and (III) are derived from the sequence set forth in SEQ ID NO: 8, Formulae (IV) and (V) are derived from the sequence set forth in SEQ ID NO: 12, and Formula (VI) is derived from the sequence set forth in SEQ ID NO: 14.

Formula (I):
(I)
X¹X²WX³X⁴X⁵GX⁶X⁷T

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, F, G, H, K, L, N, Q or R.

In some embodiments, in general Formula (I), X¹ is A or S.

In some embodiments, in general Formula (I), X² is A, D, E, F, H, I, L, N, Q, T, V or W. In some embodiments, in general Formula (I), X² is H or T.

In some embodiments, in Formula (I), X³ is A, F, H, I, S, T, V, W or Y. In some embodiments, in Formula (I), X³ is D, E, F, H, N, Q, S, T or Y. In some embodiments, in Formula (I), X³ is F, H, S, T or Y. In some embodiments, in Formula (I), X³ is E, F, H, Q, S or T. In some embodiments, in Formula (I), X³ is F, H, S or T. In some embodiments, in general Formula (I), X³ is E, F or S. In some embodiments, in general Formula (I), X³ is F or S.

In some embodiments, in Formula (I), X⁴ is D, G, I or L. In some embodiments, in Formula (I), X⁴ is D or G.

In some embodiments, in Formula (I), X⁵ is A, D, E, G, H, K or R. In some embodiments, in Formula (I), X⁵ is G.

In some embodiments, in Formula (I), X⁶ is F, W or Y. In some embodiments, in Formula (I), X⁶ is Y.

In some embodiments, in Formula (I), X⁷ is A, D, E, G, H, K, L, N, Q or R. In some embodiments, in Formula (I), X⁷ is A, F, H, K, L or N. In some embodiments, in Formula (I), X⁷ is A, H, K, L or N. In some embodiments, in Formula (I), X⁷ is A or N.

In certain embodiments, in Formula (I):
X¹ is A, D, N or S;
X² is A, D, E, F, H, I, L, N, Q, S, T, V, W or Y;
X³ is A, F, H, I, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, G, H, K, L, N, Q or R.

In certain embodiments, in Formula (I):
X is A or S;
X² is A, D, E, F, H, I, L, N, Q, T, V or W;
X³ is F, H, S, T or Y;
X⁴ is D, G, I or L;
X⁵ is G;
X⁶ is F, W or Y, and
X⁷ is A, F, H, K, L or N.

Other combinations of the foregoing embodiments described for Formula (I) are also contemplated and each combination forms a separate embodiment for the purposes of the present disclosure.

Formula (Ia):
(Ia)
X¹X²WX³X⁴X⁵GYX⁶T

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y, and
X⁶ is A, D, E, F, G, H, K, L, N, Q or R.

In some embodiments, in general Formula (Ia), X¹ is A or S.

In some embodiments, in general Formula (Ia), X² is A, D, E, F, H, I, L, N, Q, T, V or W. In some embodiments, in general Formula (Ia), X² is H or T.

In some embodiments, in Formula (Ia), X³ is A, F, H, I, S, T, V, W or Y. In some embodiments, in Formula (Ia), X³ is D, E, F, H, N, Q, S, T or Y. In some embodiments, in Formula (Ia), X³ is F, H, S, T or Y. In some embodiments, in Formula (Ia), X³ is E, F, H, Q, S or T. In some embodiments, in Formula (Ia), X³ is F, H, S or T. In some embodiments, in general Formula (I), X³ is E, F or S. In some embodiments, in general Formula (Ia), X³ is F or S.

In some embodiments, in Formula (Ia), X⁴ is D, G, I or L. In some embodiments, in Formula (Ia), X⁴ is D or G.

In some embodiments, in Formula (Ia), X⁵ is A, D, E, G, H, K or R. In some embodiments, in Formula (Ia), X⁵ is G.

In some embodiments, in Formula (Ia), X⁶ is A, D, E, G, H, K, L, N, Q or R. In some embodiments, in Formula (Ia), X⁶ is A, F, H, K, L or N. In some embodiments, in Formula (Ia), X⁶ is A, H, K, L or N. In some embodiments, in Formula (Ia), X⁶ is A or N.

Combinations of any of the foregoing embodiments described for Formula (Ia) are also contemplated and each combination forms a separate embodiment for the purposes of the present disclosure.

Formula (Ib):
(Ib)
X¹X²WX³X⁴GGYX⁵T

wherein:
X¹ is A or S;
X² is A, D, E, F, H, I, L, N, Q, T, V or W;
X³ is D, E, F, H, N, Q, S, T or Y;
X⁴ is D, G, I or L, and
X⁵ is A, F, H, K, L or N.

In some embodiments, in Formula (Ib), X² is H or T.

In some embodiments, in Formula (Ib), X³ is F, H, S or Y. In some embodiments, in Formula (Ib), X³ is E, F, H, Q, S or T. In some embodiments, in Formula (Ib), X³ is F, H or S. In some embodiments, in Formula (Ib), X³ is E, F or S. In some embodiments, in Formula (Tb), X³ is F or S.

In some embodiments, in Formula (Ib), X⁴ is D or G.

In some embodiments, in Formula (Ib), X⁵ is A, F, H, K or L. In some embodiments, in Formula (Ib), X⁵ is A or N. In some embodiments, in Formula (Ib), X⁵ is A.

Combinations of any of the foregoing embodiments described for Formula (Ib) are also contemplated and each combination forms a separate embodiment for the purposes of the present disclosure.

Formula (II):
(II)
X¹LDX²X³GKGX⁴V

wherein:
X¹ is F or G;
X² is E, H, Q or T;
X³ is E, N, R, S or T, and
X⁴ is A, Y or V.

In some embodiments, in Formula (II), X² is E.

In some embodiments, in Formula (II), X³ is E, N, R or S. In some embodiments, in Formula (II), X³ is E or N.

Combinations of any of the foregoing embodiments described for Formula (II) are also contemplated and each combination forms a separate embodiment for the purposes of the present disclosure.

Formula (III):
(III)
X¹TDEX²GKGX³T

wherein:
X¹ is F or G;
X² is E or N, and
X³ is A or V.

Formula (IV):
(IV)
X¹FX²X³X⁴X⁵GEVV

wherein:
X¹ is A or D;
X² is D or N;
X³ is D, E, H, N, P, Q, S or T;
X⁴ is D, E, N, S or T, and
X⁵ is D or Q.

In some embodiments, in Formula (IV), X¹ is D.

In some embodiments, in Formula (IV), X² is D.

In some embodiments, in Formula (IV), X³ is E, H, N, S or T.

In some embodiments, in Formula (IV), X⁴ is D, N, S or T.

Combinations of any of the foregoing embodiments described for Formula (IV) are also contemplated and each combination forms a separate embodiment for the purposes of the present disclosure.

Formula (V):
(V)
X¹TDX²X³X⁴GEVT

wherein:
X¹ is A or D;
X² is D, P or Q;
X³ is D, E or N, and
X⁴ is D or Q.

Formula (VI):
(VI)
LTDX¹X²GX³PX⁴R

wherein:
X¹ is E or H;
X² is D, E or N;
X³ is R or S, and
X⁴ is I, Q or Y.

In some embodiments, in Formula (VI), X¹ is E.

In some embodiments, in Formula (VI), X⁴ is I or Y.

Combinations of any of the foregoing embodiments described for Formula (VI) are also contemplated and each combination forms a separate embodiment for the purposes of the present disclosure.

In certain embodiments, the replacement loop comprised by the heterodimeric Fc variant is a polypeptide loop comprising an amino acid sequence as set forth in any one of the sequences shown in Tables 3A & 3B (SEQ ID NOs: 4-172). As the polypeptide loop replaces residues 325-331 in the parental Fc sequence, the following numbering system is used in Tables 3A & 3B, and throughout the description. The residue immediately following position 324 in the Fc is designated 325*, the remaining residues of the polypeptide loop are numbered sequentially from 326* to 331*. Any additional residues after 331* in the polypeptide loop are designated a letter, i.e. 331*A, 331*B, 331*C, etc.

In some embodiments, the replacement loop comprised by the heterodimeric Fc variant is a polypeptide loop comprising an amino acid sequence as set forth in any one of SEQ ID NOs: 4-90 (see Table 3A). In some embodiments, the polypeptide loop comprises an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 (Table 3A). In certain embodiments, the heterodimeric Fc variant further comprises the mutation I332L.

In certain embodiments, the replacement loop comprised by the heterodimeric Fc variant is a polypeptide loop comprising an amino acid sequence as set forth in any one of SEQ ID NOs: 6, 8, 47, 68 or 73. In certain embodiments, the heterodimeric Fc variant further comprises the mutation I332L.

TABLE 3A
Exemplary Loop Replacement Sequences

| | | | | | | | | | | | | SEQ |
|---|---|---|---|---|---|---|---|---|---|---|---|---|
| Template | Variant | | | | | | | | | | | ID |
| ID | # | 325* | 326* | 327* | 328* | 329* | 330* | 331* | 331*A | 331*B | 331*C | NO |
| Template 1 | 17898¹ | S | T | W | F | D | G | G | Y | A | T | 6 |
| (Parental) | | | | | | | | | | | | |
| Template 1 | 27389 | A | H | W | E | G | G | G | Y | N | T | 15 |
| (S325*A_{—} | | | | | | | | | | | | |
| T326*H_{—} | | | | | | | | | | | | |
| F328*E_{—} | | | | | | | | | | | | |
| D329*G_{—} | | | | | | | | | | | | |
| A331*BN) | | | | | | | | | | | | |
| Template 1 | 27390 | A | H | W | Q | G | G | G | Y | N | T | 16 |
| (S325*A_{—} | | | | | | | | | | | | |
| T326*H_{—} | | | | | | | | | | | | |
| F328*E_{—} | | | | | | | | | | | | |
| D329*G_{—} | | | | | | | | | | | | |
| A331*BN) | | | | | | | | | | | | |
| Template 1 | 26426 | S | Q | W | F | D | G | G | Y | A | T | 17 |
| (T326*Q) | | | | | | | | | | | | |
| Template 1 | 26427 | S | N | W | F | D | G | G | Y | A | T | 18 |
| (T326*N) | | | | | | | | | | | | |
| Template 1 | 26530 | S | T | W | F | D | G | G | F | A | T | 19 |
| (Y331*AF) | | | | | | | | | | | | |
| Template 1 | 26501 | S | T | W | F | D | E | G | Y | A | T | 20 |
| (G330*E) | | | | | | | | | | | | |
| Template 1 | 26500 | S | T | W | F | D | D | G | Y | A | T | 21 |
| (G330*D) | | | | | | | | | | | | |
| Template 1 | 26488 | S | T | W | F | D | A | G | Y | A | T | 22 |
| (G330*A) | | | | | | | | | | | | |
| Template 1 | 26419 | S | L | W | F | D | G | G | Y | A | T | 23 |
| (T326*L) | | | | | | | | | | | | |
| Template 1 | 26420 | S | I | W | F | D | G | G | Y | A | T | 24 |
| (T326*I) | | | | | | | | | | | | |
| Template 1 | 26429 | S | E | W | F | D | G | G | Y | A | T | 25 |
| (T326*E) | | | | | | | | | | | | |
| Template 1 | 26428 | S | D | W | F | D | G | G | Y | A | T | 26 |
| (T326*D) | | | | | | | | | | | | |
| Template 1 | 26417 | S | A | W | F | D | G | G | Y | A | T | 27 |
| (T326*A) | | | | | | | | | | | | |
| Template 1 | 26422 | S | F | W | F | D | G | G | Y | A | T | 28 |
| (T326*F) | | | | | | | | | | | | |
| Template 1 | 20974¹ | S | H | W | T | D | G | G | Y | A | T | 29 |
| (T326*H_{—} | | | | | | | | | | | | |
| F328*T) | | | | | | | | | | | | |
| Template 1 | 27381 | S | H | W | S | D | G | G | Y | N | T | 30 |
| (T326*H_{—} | | | | | | | | | | | | |
| F328*S_{—} | | | | | | | | | | | | |
| A331*BN) | | | | | | | | | | | | |
| Template 1 | 20972¹ | S | H | W | S | D | G | G | Y | A | T | 31 |
| (T326*H_{—} | | | | | | | | | | | | |
| F328*S) | | | | | | | | | | | | |
| Template 1 | 27384 | S | H | W | Q | G | G | G | Y | N | T | 32 |
| (T326*H_{—} | | | | | | | | | | | | |
| F328*Q_{—} | | | | | | | | | | | | |
| D329*G_{—} | | | | | | | | | | | | |
| A331*BN) | | | | | | | | | | | | |
| Template 1 | 20965¹ | S | H | W | Q | G | G | G | Y | A | T | 33 |
| (T326*H_{—} | | | | | | | | | | | | |
| F328*Q_{—} | | | | | | | | | | | | |
| D329*G) | | | | | | | | | | | | |
| Template 1 | 20966¹ | S | H | W | Q | D | G | G | Y | A | T | 34 |
| (T326*H | | | | | | | | | | | | |
| F328*Q) | | | | | | | | | | | | |
| Template 1 | 20968¹ | S | H | W | N | D | G | G | Y | A | T | 35 |
| (T326*H_{—} | | | | | | | | | | | | |
| F328*N) | | | | | | | | | | | | |
| Template 1 | 20969¹ | S | H | W | H | G | G | G | Y | A | T | 36 |
| (T326*H_{—} | | | | | | | | | | | | |
| F328*H_{—} | | | | | | | | | | | | |
| D329*G) | | | | | | | | | | | | |
| Template 1 | 20970¹ | S | H | W | H | D | G | G | Y | A | T | 37 |
| (T326*H_{—} | | | | | | | | | | | | |
| F328*H) | | | | | | | | | | | | |
| Template 1 | 20964¹ | S | H | W | F | D | G | G | Y | A | T | 38 |
| (T326*H) | | | | | | | | | | | | |
| Template 1 | 27383 | S | H | W | E | G | G | G | Y | N | T | 39 |
| (T326*H_{—} | | | | | | | | | | | | |
| F328*E_{—} | | | | | | | | | | | | |
| D329*G_{—} | | | | | | | | | | | | |
| A331*BN) | | | | | | | | | | | | |
| Template 1 | 20975¹ | S | H | W | E | G | G | G | Y | A | T | 40 |
| (T326*H_{—} | | | | | | | | | | | | |
| F328*E_{—} | | | | | | | | | | | | |
| D329*G) | | | | | | | | | | | | |
| Template 1 | 20978¹ | S | H | W | D | D | G | G | Y | A | T | 41 |
| (T326*H_{—} | | | | | | | | | | | | |
| F328*D) | | | | | | | | | | | | |
| Template 1 | 27385 | S | H | W | F | D | G | G | Y | N | T | 42 |
| (T326*H_{—} | | | | | | | | | | | | |
| A331*BN) | | | | | | | | | | | | |
| Template 1 | 21005¹ | S | T | W | H | G | G | G | Y | A | T | 43 |
| (F328*H_{—} | | | | | | | | | | | | |
| D329*G) | | | | | | | | | | | | |
| Template 1 | 21006¹ | S | T | W | H | D | G | G | Y | A | T | 44 |
| (F328*H) | | | | | | | | | | | | |
| Template 1 | 21001¹ | S | T | W | Q | G | G | G | Y | A | T | 45 |
| (F328*Q_{—} | | | | | | | | | | | | |
| D329*G) | | | | | | | | | | | | |
| Template 1 | 26473 | S | T | W | F | L | G | G | Y | A | T | 46 |
| (D329*L) | | | | | | | | | | | | |
| Template 1 | 26474¹ | S | T | W | F | I | G | G | Y | A | T | 47 |
| (D329*I) | | | | | | | | | | | | |
| Template 1 | 21008¹ | S | T | W | S | D | G | G | Y | A | T | 48 |
| (F328*S) | | | | | | | | | | | | |
| Template 1 | 27386 | S | T | W | S | D | G | G | Y | N | T | 49 |
| (F328*S_{—} | | | | | | | | | | | | |
| A331*BN) | | | | | | | | | | | | |
| Template 1 | 27379 | A | H | W | F | D | G | G | Y | A | T | 50 |
| (S325*A_{—} | | | | | | | | | | | | |
| T326*H) | | | | | | | | | | | | |
| Template 1 | 27391 | A | H | W | F | D | G | G | Y | N | T | 51 |
| (S325*A_{—} | | | | | | | | | | | | |
| T326*H_{—} | | | | | | | | | | | | |
| A331*BN) | | | | | | | | | | | | |
| Template 1 | 27378 | A | H | W | Q | G | G | G | Y | A | T | 52 |
| (S325*A_{—} | | | | | | | | | | | | |
| T326*H_{—} | | | | | | | | | | | | |
| F328*Q_{—} | | | | | | | | | | | | |
| D329*G) | | | | | | | | | | | | |
| Template 1 | 27375 | A | H | W | S | D | G | G | Y | A | T | 53 |
| (S325*A_{—} | | | | | | | | | | | | |
| T326*H_{—} | | | | | | | | | | | | |
| F328*S) | | | | | | | | | | | | |
| Template 1 | 27387 | A | H | W | S | D | G | G | Y | N | T | 54 |
| (S325*A_{—} | | | | | | | | | | | | |
| T326*H_{—} | | | | | | | | | | | | |
| F328*S_{—} | | | | | | | | | | | | |
| A331*BN) | | | | | | | | | | | | |
| Template 1 | 20505¹ | A | T | W | F | D | G | G | Y | A | T | 55 |
| (S325*A) | | | | | | | | | | | | |
| Template 1 | 27374 | A | T | W | F | D | G | G | Y | N | T | 56 |
| (S325*A_{—} | | | | | | | | | | | | |
| A331*BN) | | | | | | | | | | | | |
| Template 1 | 26423 | S | W | W | F | D | G | G | Y | A | T | 57 |
| (T326*W) | | | | | | | | | | | | |
| Template 1 | 26418 | S | V | W | F | D | G | G | Y | A | T | 58 |
| (T326*V) | | | | | | | | | | | | |
| Template 1 | 26459 | S | T | W | Y | D | G | G | Y | A | T | 59 |
| (F328*Y) | | | | | | | | | | | | |
| Template 1 | 21007¹ | S | T | W | S | G | G | G | Y | A | T | 60 |
| (F328*S_{—} | | | | | | | | | | | | |
| D329*G) | | | | | | | | | | | | |
| Template 1 | 20999¹ | S | T | W | F | G | G | G | Y | A | T | 61 |
| (D329*G) | | | | | | | | | | | | |
| Template 1 | 27392 | A | T | W | S | D | G | G | Y | N | T | 62 |
| (S325*A_{—} | | | | | | | | | | | | |
| F328*S_{—} | | | | | | | | | | | | |
| A331*BN) | | | | | | | | | | | | |
| Template 1 | 20500¹ | S | T | W | F | D | G | G | Y | L | T | 63 |
| (A331*BL) | | | | | | | | | | | | |
| Template 1 | 26556 | S | T | W | F | D | G | G | Y | K | T | 64 |
| (A331*BK) | | | | | | | | | | | | |
| Template 1 | 26557 | S | T | W | F | D | G | G | Y | H | T | 65 |
| (A331*BH) | | | | | | | | | | | | |
| Template 1 | 26546 | S | T | W | F | D | G | G | Y | F | T | 66 |
| (A331*BF) | | | | | | | | | | | | |
| Template 1 | 26531 | S | T | W | F | D | G | G | W | A | T | 67 |
| (Y331*AW) | | | | | | | | | | | | |
| Template 1 | 26503¹ | S | T | W | F | D | K | G | Y | A | T | 68 |
| (G330*K) | | | | | | | | | | | | |
| Template 1 | 26502 | S | T | W | F | D | R | G | Y | A | T | 69 |
| (G330*R) | | | | | | | | | | | | |
| Template 1 | 26504 | S | T | W | F | D | H | G | Y | A | T | 70 |
| (G330*H) | | | | | | | | | | | | |
| Template 7 | 19216¹ | G | L | D | E | E | G | K | G | A | V | 8 |
| (Parental) | | | | | | | | | | | | |
| Template 7 | 27456 | G | L | D | Q | S | G | K | G | Y | V | 71 |
| (E328*Q_{—} | | | | | | | | | | | | |
| E329*S_{—} | | | | | | | | | | | | |
| A331*BY) | | | | | | | | | | | | |
| Template 7 | 27454 | G | L | D | T | N | G | K | G | Y | V | 72 |
| (E328*T_{—} | | | | | | | | | | | | |
| E329*N_{—} | | | | | | | | | | | | |
| A331*BY) | | | | | | | | | | | | |
| Template 7 | 27455¹ | G | L | D | H | R | G | K | G | Y | V | 73 |
| (E328*H_{—} | | | | | | | | | | | | |
| E329*R_{—} | | | | | | | | | | | | |
| A331*BY) | | | | | | | | | | | | |
| Template 7 | 27462 | F | L | D | T | N | G | K | G | V | V | 74 |
| (G325*F_{—} | | | | | | | | | | | | |
| E328*T_{—} | | | | | | | | | | | | |
| E329*N_{—} | | | | | | | | | | | | |
| A331*BV) | | | | | | | | | | | | |
| Template 7 | 27464 | F | L | D | Q | S | G | K | G | V | V | 75 |
| (G325*F_{—} | | | | | | | | | | | | |
| E328*Q_{—} | | | | | | | | | | | | |
| E329*S_{—} | | | | | | | | | | | | |
| A331*BV) | | | | | | | | | | | | |
| Template 7 | 27463 | F | L | D | H | R | G | K | G | V | V | 76 |
| (G325*F_{—} | | | | | | | | | | | | |
| E328*H_{—} | | | | | | | | | | | | |
| E329*R_{—} | | | | | | | | | | | | |
| A331*BV) | | | | | | | | | | | | |
| Template 7 | 27461 | F | L | D | E | N | G | K | G | V | V | 77 |
| (G325*F_{—} | | | | | | | | | | | | |
| E329*N_{—} | | | | | | | | | | | | |
| A331*BV) | | | | | | | | | | | | |
| Template 7 | 27453 | G | L | D | E | N | G | K | G | Y | V | 78 |
| (E329*N_{—} | | | | | | | | | | | | |
| A331*BY) | | | | | | | | | | | | |
| Template | 19218¹ | D | F | D | Q | N | Q | G | E | V | V | 12 |
| 66 | | | | | | | | | | | | |
| (Parental) | | | | | | | | | | | | |
| Template 66 | 20639¹ | D | F | N | H | N | D | G | E | V | V | 79 |
| (D327*N_{—} | | | | | | | | | | | | |
| Q328*H_{—} | | | | | | | | | | | | |
| Q330*D) | | | | | | | | | | | | |
| Template 66 | 20749¹ | D | F | D | T | D | D | G | E | V | V | 80 |
| (Q328*T_{—} | | | | | | | | | | | | |
| N329*D_{—} | | | | | | | | | | | | |
| Q330*D) | | | | | | | | | | | | |
| Template 66 | 20732¹ | D | F | D | S | T | Q | G | E | V | V | 81 |
| (Q328*S_{—} | | | | | | | | | | | | |
| N329*T) | | | | | | | | | | | | |
| Template 66 | 20733¹ | D | F | D | S | T | D | G | E | V | V | 82 |
| (Q328*S_{—} | | | | | | | | | | | | |
| N329*T_{—} | | | | | | | | | | | | |
| Q330*D) | | | | | | | | | | | | |
| Template 66 | 20742¹ | D | F | D | T | S | Q | G | E | V | V | 83 |
| (Q328*T_{—} | | | | | | | | | | | | |
| N329*S) | | | | | | | | | | | | |
| Template 66 | 20724¹ | D | F | D | H | D | Q | G | E | V | V | 84 |
| (Q328*H_{—} | | | | | | | | | | | | |
| N329*D) | | | | | | | | | | | | |
| Template 66 | 20713¹ | D | F | D | N | D | D | G | E | V | V | 85 |
| (Q328*N_{—} | | | | | | | | | | | | |
| N329*D_{—} | | | | | | | | | | | | |
| Q330*D) | | | | | | | | | | | | |
| Template 66 | 20761¹ | D | F | D | E | D | D | G | E | V | V | 86 |
| (Q328*E_{—} | | | | | | | | | | | | |
| N329*D_{—} | | | | | | | | | | | | |
| Q330*D) | | | | | | | | | | | | |
| Template | 19221¹ | L | T | D | E | E | G | R | P | Y | R | 14 |
| 151 | | | | | | | | | | | | |
| (Parental) | | | | | | | | | | | | |
| Template | 27471 | L | T | D | H | N | G | R | P | I | R | 87 |
| 151 | | | | | | | | | | | | |
| (E328*H_{—} | | | | | | | | | | | | |
| E329*N_{—} | | | | | | | | | | | | |
| Y331*BI) | | | | | | | | | | | | |
| Template | 20328¹ | L | T | D | E | E | G | R | P | I | R | 88 |
| 151 | | | | | | | | | | | | |
| (Y331*BI) | | | | | | | | | | | | |
| Template | 27474 | L | T | D | E | D | G | S | P | I | R | 89 |
| 151 | | | | | | | | | | | | |
| (E329*D_{—} | | | | | | | | | | | | |
| R331*S_{—} | | | | | | | | | | | | |
| Y331*BI) | | | | | | | | | | | | |
| Template | 27472 | L | T | D | E | D | G | R | P | I | R | 90 |
| 151 | | | | | | | | | | | | |
| (E329*D_{—} | | | | | | | | | | | | |
| Y331*BI) | | | | | | | | | | | | |
| Template | | V | T | W | E | D | G | K | S | E | R | 9 |
| 19 | | | | | | | | | | | | |
| Template | | W | T | D | Q | S | G | Q | D | R | — | 4 |
| 231 | | | | | | | | | | | | |
| Template | | L | D | M | E | G | R | K | I | H | — | 5 |
| 168 | | | | | | | | | | | | |
| Template | | H | F | D | E | N | G | E | I | V | T | 7 |
| 11 | | | | | | | | | | | | |
| Template | | L | I | D | E | N | G | N | E | Q | K | 10 |
| 38 | | | | | | | | | | | | |
| Template | | V | Q | D | A | T | G | A | P | F | L | 11 |
| 60 | | | | | | | | | | | | |
| Template | | S | D | F | E | G | K | P | T | L | — | 13 |
| 83 | | | | | | | | | | | | |

¹Also used in other variants. Representative variant # provided.

TABLE 3B
Exemplary Loop Replacement Sequences

| | | | | | | | | | | | | SEQ |
|---|---|---|---|---|---|---|---|---|---|---|---|---|
| Template | Variant | | | | | | | | | | | ID |
| ID | # | 325* | 326* | 327* | 328* | 329* | 330* | 331* | 331*A | 331*B | 331*C | NO |
| Template 1 | 17898¹ | S | T | W | F | D | G | G | Y | A | T | 6 |
| (Parental) | | | | | | | | | | | | |
| Template 1 | 26425 | S | S | W | F | D | G | G | Y | A | T | 91 |
| (T326*S) | | | | | | | | | | | | |
| Template 1 | 26536 | S | T | W | F | D | G | G | E | A | T | 92 |
| (Y331*AE) | | | | | | | | | | | | |
| Template 1 | 26535 | S | T | W | F | D | G | G | D | A | T | 93 |
| (Y331*AD) | | | | | | | | | | | | |
| Template 1 | 26525 | S | T | W | F | D | G | G | A | A | T | 94 |
| (Y331*AA) | | | | | | | | | | | | |
| Template 1 | 26453 | S | T | W | A | D | G | G | Y | A | T | 95 |
| (F328*A) | | | | | | | | | | | | |
| Template 1 | 27397 | S | T | T | H | G | G | G | Y | A | T | 96 |
| (W327*T_{—} | | | | | | | | | | | | |
| F328*H_{—} | | | | | | | | | | | | |
| D329*G) | | | | | | | | | | | | |
| Template 1 | 26409 | N | T | W | F | D | G | G | Y | A | T | 97 |
| (S325*N) | | | | | | | | | | | | |
| Template 1 | 26539 | S | T | W | F | D | G | G | H | A | T | 98 |
| (Y331*AH) | | | | | | | | | | | | |
| Template 1 | 27382 | S | H | W | E | D | G | G | Y | N | T | 99 |
| (T326*H_{—} | | | | | | | | | | | | |
| F328*E_{—} | | | | | | | | | | | | |
| A331*BN) | | | | | | | | | | | | |
| Template 1 | 20976¹ | S | H | W | E | D | G | G | Y | A | T | 100 |
| (T326*H_{—} | | | | | | | | | | | | |
| F328*E) | | | | | | | | | | | | |
| Template 1 | 19410¹ | D | T | W | F | D | G | G | Y | A | T | 101 |
| (S325*D) | | | | | | | | | | | | |
| Template 1 | 27398 | S | H | T | T | G | G | G | Y | A | T | 102 |
| (T326*H_{—} | | | | | | | | | | | | |
| W327*T_{—} | | | | | | | | | | | | |
| F328*T_{—} | | | | | | | | | | | | |
| D329*G) | | | | | | | | | | | | |
| Template 1 | 27396 | S | H | T | H | G | G | G | Y | A | T | 103 |
| (T326*H_{—} | | | | | | | | | | | | |
| W327*T_{—} | | | | | | | | | | | | |
| F328*H_{—} | | | | | | | | | | | | |
| D329*G) | | | | | | | | | | | | |
| Template 1 | 20955¹ | S | H | D | T | G | G | G | Y | A | T | 104 |
| (T326*H_{—} | | | | | | | | | | | | |
| W327*D_{—} | | | | | | | | | | | | |
| F328*T_{—} | | | | | | | | | | | | |
| D329*G) | | | | | | | | | | | | |
| Template 1 | 26456 | S | T | W | I | D | G | G | Y | A | T | 105 |
| (F328*I) | | | | | | | | | | | | |
| Template 1 | 26481 | S | T | W | F | Q | G | G | Y | A | T | 106 |
| (D329*Q) | | | | | | | | | | | | |
| Template 1 | 26487 | S | T | W | F | P | G | G | Y | A | T | 107 |
| (D329*P) | | | | | | | | | | | | |
| Template 1 | 27388 | A | H | W | E | D | G | G | Y | N | T | 108 |
| (S325*A_{—} | | | | | | | | | | | | |
| T326*H_{—} | | | | | | | | | | | | |
| F328*E_{—} | | | | | | | | | | | | |
| A331*BN) | | | | | | | | | | | | |
| Template 1 | 27377 | A | H | W | E | G | G | G | Y | A | T | 109 |
| (S325*A_{—} | | | | | | | | | | | | |
| T326*H_{—} | | | | | | | | | | | | |
| F328*E_{—} | | | | | | | | | | | | |
| D329*G) | | | | | | | | | | | | |
| Template 1 | 26424 | S | Y | W | F | D | G | G | Y | A | T | 110 |
| (T326*Y) | | | | | | | | | | | | |
| Template 1 | 26458 | S | T | W | W | D | G | G | Y | A | T | 111 |
| (F328*W) | | | | | | | | | | | | |
| Template 1 | 26454 | S | T | W | V | D | G | G | Y | A | T | 112 |
| (F328*V) | | | | | | | | | | | | |
| Template 1 | 21010¹ | S | T | W | T | D | G | G | Y | A | T | 113 |
| (F328*T) | | | | | | | | | | | | |
| Template 1 | 26483 | S | T | W | F | E | G | G | Y | A | T | 114 |
| (D329*E) | | | | | | | | | | | | |
| Template 1 | 26555 | S | T | W | F | D | G | G | Y | R | T | 115 |
| (A331*BR) | | | | | | | | | | | | |
| Template 1 | 26551 | S | T | W | F | D | G | G | Y | Q | T | 116 |
| (A331*BQ) | | | | | | | | | | | | |
| Template 1 | 20499¹ | S | T | W | F | D | G | G | Y | N | T | 117 |
| (A331*BN) | | | | | | | | | | | | |
| Template 1 | 26541 | S | T | W | F | D | G | G | Y | G | T | 118 |
| (A331*BG) | | | | | | | | | | | | |
| Template 1 | 26554 | S | T | W | F | D | G | G | Y | E | T | 119 |
| (A331*BE) | | | | | | | | | | | | |
| Template 1 | 26553 | S | T | W | F | D | G | G | Y | D | T | 120 |
| (A331*BD) | | | | | | | | | | | | |
| Template 1 | 26540 | S | T | W | F | D | G | G | P | A | T | 121 |
| (Y331*AP) | | | | | | | | | | | | |
| Template 1 | 26497 | S | T | W | F | D | S | G | Y | A | T | 122 |
| (G330*S) | | | | | | | | | | | | |
| Template 1 | 26499 | S | T | W | F | D | N | G | Y | A | T | 123 |
| (G330*N) | | | | | | | | | | | | |
| Template 1 | 26496 | S | T | W | F | D | T | G | Y | A | T | 124 |
| (G330*T) | | | | | | | | | | | | |
| Template 1 | 26495 | S | T | W | F | D | Y | G | Y | A | T | 125 |
| (G330*Y) | | | | | | | | | | | | |
| Template 7 | 19216¹ | G | L | D | E | E | G | K | G | A | V | 8 |
| (Parental) | | | | | | | | | | | | |
| Template 7 | 27448 | F | L | D | E | E | G | K | G | V | V | 126 |
| (G325*F_{—} | | | | | | | | | | | | |
| A330*BV) | | | | | | | | | | | | |
| Template 7 | 27452 | G | L | D | Q | S | G | K | G | V | V | 127 |
| (E328*Q_{—} | | | | | | | | | | | | |
| E329*S_{—} | | | | | | | | | | | | |
| A330*BV) | | | | | | | | | | | | |
| Template 7 | 20834¹ | G | L | D | Q | S | G | K | G | A | V | 128 |
| (E328*Q_{—} | | | | | | | | | | | | |
| E329*S) | | | | | | | | | | | | |
| Template 7 | 20851¹ | G | L | D | H | T | G | K | G | A | V | 129 |
| (E328*H_{—} | | | | | | | | | | | | |
| E329*T) | | | | | | | | | | | | |
| Template 7 | 27450 | G | L | D | T | N | G | K | G | V | V | 130 |
| (E328*T_{—} | | | | | | | | | | | | |
| E329*N_{—} | | | | | | | | | | | | |
| A330*BV) | | | | | | | | | | | | |
| Template 7 | 20864¹ | G | L | D | T | N | G | K | G | A | V | 131 |
| (E328*T_{—} | | | | | | | | | | | | |
| E329*N) | | | | | | | | | | | | |
| Template 7 | 20464¹ | F | L | D | E | E | G | K | G | A | V | 132 |
| (G325*F) | | | | | | | | | | | | |
| Template 7 | 20846¹ | G | L | D | H | R | G | K | G | A | V | 133 |
| (E328*H_{—} | | | | | | | | | | | | |
| E329*R) | | | | | | | | | | | | |
| Template 7 | 27458 | F | L | D | T | N | G | K | G | A | V | 134 |
| (G325*F_{—} | | | | | | | | | | | | |
| E328*T_{—} | | | | | | | | | | | | |
| E329*N) | | | | | | | | | | | | |
| Template 7 | 27460 | F | L | D | Q | S | G | K | G | A | V | 135 |
| (G325*F_{—} | | | | | | | | | | | | |
| E328*Q_{—} | | | | | | | | | | | | |
| E329*S) | | | | | | | | | | | | |
| Template 7 | 27459 | F | L | D | H | R | G | K | G | A | V | 136 |
| (G325*F_{—} | | | | | | | | | | | | |
| E328*H_{—} | | | | | | | | | | | | |
| E329*R) | | | | | | | | | | | | |
| Template 7 | 27457 | F | L | D | E | N | G | K | G | A | V | 137 |
| (G325*F_{—} | | | | | | | | | | | | |
| E329*N) | | | | | | | | | | | | |
| Template 7 | 27451 | G | L | D | H | R | G | K | G | V | V | 138 |
| (E328*H_{—} | | | | | | | | | | | | |
| E329*R_{—} | | | | | | | | | | | | |
| A331*BV) | | | | | | | | | | | | |
| Template 7 | 27449 | G | L | D | E | N | G | K | G | V | V | 139 |
| (E329*N_{—} | | | | | | | | | | | | |
| A331*BV) | | | | | | | | | | | | |
| Template 7 | 20872¹ | G | L | D | E | N | G | K | G | A | V | 140 |
| (E329*N) | | | | | | | | | | | | |
| Template 7 | 20459¹ | G | L | D | E | E | G | K | G | Y | V | 141 |
| (A331*BY) | | | | | | | | | | | | |
| Template 7 | 20458¹ | G | L | D | E | E | G | K | G | V | V | 142 |
| (A331*BV) | | | | | | | | | | | | |
| Template 7- | | G | T | D | E | E | G | K | G | A | T | 143 |
| HF | | | | | | | | | | | | |
| (Parental) | | | | | | | | | | | | |
| Template 7- | 27488 | G | T | D | E | N | G | K | G | V | T | 144 |
| HF | | | | | | | | | | | | |
| (E329*N_{—} | | | | | | | | | | | | |
| A331*BV) | | | | | | | | | | | | |
| Template 7- | 27484 | G | T | D | E | N | G | K | G | A | T | 145 |
| HF | | | | | | | | | | | | |
| (E329*N) | | | | | | | | | | | | |
| Template 7- | 27485 | G | T | D | E | E | G | K | G | V | T | 146 |
| HF | | | | | | | | | | | | |
| (A331*BV) | | | | | | | | | | | | |
| Template 7- | 27487 | F | T | D | E | E | G | K | G | V | T | 147 |
| HF | | | | | | | | | | | | |
| (G325*F_{—} | | | | | | | | | | | | |
| A331*BV) | | | | | | | | | | | | |
| Template 7- | 27486 | F | T | D | E | E | G | K | G | A | T | 148 |
| HF | | | | | | | | | | | | |
| (G325*F) | | | | | | | | | | | | |
| Template | 19218¹ | D | F | D | Q | N | Q | G | E | V | V | 12 |
| 66 | | | | | | | | | | | | |
| (Parental) | | | | | | | | | | | | |
| Template 66 | 27429, | A | F | D | P | D | Q | G | E | V | V | 149 |
| (D325*A_{—} | 27435 | | | | | | | | | | | |
| Q328*P_{—} | | | | | | | | | | | | |
| N329*D) | | | | | | | | | | | | |
| Template 66 | 27428¹ | A | F | D | D | E | D | G | E | V | V | 150 |
| (D325*A_{—} | | | | | | | | | | | | |
| Q328*D_{—} | | | | | | | | | | | | |
| N329*E_{—} | | | | | | | | | | | | |
| Q330*D) | | | | | | | | | | | | |
| Template 66 | 20674¹ | D | F | N | D | E | Q | G | E | V | V | 151 |
| (D327*N_{—} | | | | | | | | | | | | |
| Q328*D_{—} | | | | | | | | | | | | |
| N329*E) | | | | | | | | | | | | |
| Template 66 | 20758¹ | D | F | D | E | E | Q | G | E | V | V | 152 |
| (Q328*E_{—} | | | | | | | | | | | | |
| N329*E) | | | | | | | | | | | | |
| Template 66 | 27436¹ | A | F | D | E | D | D | G | E | V | V | 153 |
| (D325*A_{—} | | | | | | | | | | | | |
| Q328*E_{—} | | | | | | | | | | | | |
| N329*D_{—} | | | | | | | | | | | | |
| Q330*D) | | | | | | | | | | | | |
| Template 66 | 27431¹ | A | F | D | E | E | Q | G | E | V | V | 154 |
| (D325*A_{—} | | | | | | | | | | | | |
| Q328*E_{—} | | | | | | | | | | | | |
| N329*E) | | | | | | | | | | | | |
| Template 66 | 27432¹ | A | F | D | H | D | Q | G | E | V | V | 155 |
| (D325*A_{—} | | | | | | | | | | | | |
| Q328*H_{—} | | | | | | | | | | | | |
| N329*D) | | | | | | | | | | | | |
| Template 66 | 20434¹ | A | F | D | Q | N | Q | G | E | V | V | 156 |
| (D325*A) | | | | | | | | | | | | |
| Template 66 | 27439¹ | A | F | D | S | T | D | G | E | V | V | 157 |
| (D325*A_{—} | | | | | | | | | | | | |
| Q328*S_{—} | | | | | | | | | | | | |
| N329*T_{—} | | | | | | | | | | | | |
| Q330*D) | | | | | | | | | | | | |
| Template 66 | 20766¹ | D | F | D | D | S | Q | G | E | V | V | 158 |
| (Q328*D_{—} | | | | | | | | | | | | |
| N329*S) | | | | | | | | | | | | |
| Template 66 | 20771¹ | D | F | D | D | E | D | G | E | V | V | 159 |
| (Q328*D_{—} | | | | | | | | | | | | |
| N329*E_{—} | | | | | | | | | | | | |
| Q330*D) | | | | | | | | | | | | |
| Template 66 | 20688¹ | D | F | D | P | D | Q | G | E | V | V | 160 |
| (Q328*P_{—} | | | | | | | | | | | | |
| N329*D) | | | | | | | | | | | | |
| Template | | D | T | D | Q | N | Q | G | E | V | T | 161 |
| 66-HF | | | | | | | | | | | | |
| (Parental) | | | | | | | | | | | | |
| Template | 27475 | D | T | D | D | E | D | G | E | V | T | 162 |
| 66-HF | | | | | | | | | | | | |
| (Q328*D_{—} | | | | | | | | | | | | |
| N329*E_{—} | | | | | | | | | | | | |
| Q330*D) | | | | | | | | | | | | |
| Template | 27482 | A | T | D | D | E | D | G | E | V | T | 163 |
| 66-HF | | | | | | | | | | | | |
| (D325*A_{—} | | | | | | | | | | | | |
| Q328*D_{—} | | | | | | | | | | | | |
| N329*E_{—} | | | | | | | | | | | | |
| Q330*D) | | | | | | | | | | | | |
| Template | 27483 | A | T | D | P | D | Q | G | E | V | T | 164 |
| 66-HF | | | | | | | | | | | | |
| (D325*A_{—} | | | | | | | | | | | | |
| Q328*P_{—} | | | | | | | | | | | | |
| N329*D) | | | | | | | | | | | | |
| Template | 27478¹ | A | T | D | Q | N | Q | G | E | V | T | 165 |
| 66-HF | | | | | | | | | | | | |
| (D325*A) | | | | | | | | | | | | |
| Template | 27476¹ | D | T | D | P | D | Q | G | E | V | T | 166 |
| 66-HF | | | | | | | | | | | | |
| (Q328*P_{—} | | | | | | | | | | | | |
| N329*D) | | | | | | | | | | | | |
| Template | 19221¹ | L | T | D | E | E | G | R | P | Y | R | 14 |
| 151 | | | | | | | | | | | | |
| (Parental) | | | | | | | | | | | | |
| Template | 20331¹ | L | T | D | E | E | G | S | P | Y | R | 167 |
| 151 | | | | | | | | | | | | |
| (R331*S) | | | | | | | | | | | | |
| Template | 20576¹ | L | T | D | H | N | G | R | P | Y | R | 168 |
| 151 | | | | | | | | | | | | |
| (E328*H_{—} | | | | | | | | | | | | |
| E329*N) | | | | | | | | | | | | |
| Template | 27473 | L | T | D | E | D | G | S | P | Y | R | 169 |
| 151 | | | | | | | | | | | | |
| (E329*D_{—} | | | | | | | | | | | | |
| R331*S) | | | | | | | | | | | | |
| Template | 20602¹ | L | T | D | E | D | G | R | P | Y | R | 170 |
| 151 | | | | | | | | | | | | |
| (E329*D) | | | | | | | | | | | | |
| Template | 20319¹ | L | T | D | E | E | G | R | P | Q | R | 171 |
| 151 | | | | | | | | | | | | |
| (Y331*BQ) | | | | | | | | | | | | |
| Template | | V | T | W | E | D | G | K | S | E | R | 9 |
| 19 | | | | | | | | | | | | |
| (Parental) | | | | | | | | | | | | |
| Template 19 | 27465 | A | T | W | E | D | G | K | S | E | R | 172 |
| (V325*A) | | | | | | | | | | | | |

¹Also used in other variants. Representative variant # provided.

### Additional CH2 Domain Mutations

In certain embodiments, the heterodimeric Fc variant comprises an asymmetric loop replacement as described in any one of the embodiments above and one or more additional mutations in the CH2 domain. The one or more additional mutations in the CH2 domain may be symmetric mutations or asymmetric mutations and may increase the selectivity of the heterodimeric Fc variant for FcγRIIb, or increase the affinity of the heterodimeric Fc variant for FcγRIIb, or increase both the selectivity and affinity of the heterodimeric Fc variant for FcγRIIb. In some embodiments, the heterodimeric Fc variant comprises an asymmetric loop replacement as described in any one of the embodiments above and one or more additional asymmetric mutations in the CH2 domain.

In certain embodiments, the heterodimeric Fc variant comprises between one and 20 amino acid mutations in the CH2 domain, one of which is an asymmetric loop replacement. In some embodiments, the heterodimeric Fc variant comprises an asymmetric loop replacement and between one and 15 additional amino acid mutations in the CH2 domain. In some embodiments, the heterodimeric Fc variant comprises an asymmetric loop replacement and between one and 12 additional amino acid mutations in the CH2 domain, for example, between one and 11 additional amino acid mutations, between one and 10 additional amino acid mutations, between one and 9 additional amino acid mutations or between one and 8 additional amino acid mutations in the CH2 domain.

Reference to an “asymmetric loop replacement” or “loop replacement” above and in the embodiments described below in combination with one or more additional amino acid mutations in the CH2 domain is intended to encompass an asymmetric loop replacement as described in any one of the embodiments detailed above under “Asymmetric Loop Replacement” and each combination forms an embodiment of the present disclosure to the same extent as if each combination were individually described.

In certain embodiments, the heterodimeric Fc variant comprises an asymmetric loop replacement and a mutation at position 236 in the CH2 domain. The mutation at position 236 may be a symmetric mutation or an asymmetric mutation. In certain embodiments, the heterodimeric Fc variant comprises an asymmetric loop replacement, a mutation at position 236 and one or more additional mutations in the CH2 domain.

In some embodiments, the heterodimeric Fc variant comprises a loop replacement in one Fc polypeptide and a mutation at position 236 in the same Fc polypeptide. In some embodiments, the heterodimeric Fc variant comprises a loop replacement in one Fc polypeptide and a mutation at position 236 in the other Fc polypeptide. In some embodiments, the heterodimeric Fc variant comprises a loop replacement in one Fc polypeptide and a mutation at position 236 in both Fc polypeptides. In some embodiments, the heterodimeric Fc variant comprises a loop replacement in one Fc polypeptide and a mutation at position 236 in both Fc polypeptides, where the mutation at position 236 is symmetric (i.e. the mutation at position 236 is the same in both Fc polypeptides). In some embodiments, the heterodimeric Fc variant comprises a loop replacement in one Fc polypeptide and a mutation at position 236 in both Fc polypeptides, where the mutation at position 236 is asymmetric (i.e. the mutation at position 236 is different in each Fc polypeptide, or one Fc polypeptide comprises a mutation at position 236 and the other Fc polypeptide does not include a mutation at position 236).

In certain embodiments, the heterodimeric Fc variant comprises a loop replacement in one Fc polypeptide and a mutation at position 236 in the same Fc polypeptide selected from G236D, G236E, G236K, G236N and G236T. In some embodiments, the heterodimeric Fc variant comprises a loop replacement in one Fc polypeptide and a mutation at position 236 in the same Fc polypeptide selected from G236D and G236N.

In certain embodiments, the heterodimeric Fc variant comprises a loop replacement in one Fc polypeptide and a mutation at position 236 in the other Fc polypeptide selected from G236A, G236D, G236E, G236F, G236H, G236I, G236L, G236N, G236P, G236Q, G236S, G236T, G236V, G236W and G236Y. In some embodiments, the heterodimeric Fc variant comprises a loop replacement in one Fc polypeptide and a mutation at position 236 in the other Fc polypeptide selected from G236D, G236K and G236N.

In certain embodiments, the heterodimeric Fc variant comprises a loop replacement in one Fc polypeptide and a mutation at position 236 in both Fc polypeptides. In some embodiments, the first Fc polypeptide of the heterodimeric Fc variant comprises a mutation at position 236 selected from G236A, G236D, G236E, G236F, G236H, G236I, G236L, G236N, G236P, G236Q, G236S, G236T, G236V, G236W and G236Y, and the second Fc polypeptide of the heterodimeric Fc variant comprises a loop replacement and a mutation at position 236 selected from G236D, G236E, G236K, G236N and G236T. In some embodiments, the first Fc polypeptide of the heterodimeric Fc variant comprises a mutation at position 236 selected from G236A, G236D, G236E, G236F, G236H, G236I, G236L, G236N, G236P, G236Q, G236S, G236T, G236V, G236W and G236Y, and the second Fc polypeptide of the heterodimeric Fc variant comprises a loop replacement and the mutation G236D. In some embodiments, the first Fc polypeptide of the heterodimeric Fc variant comprises the mutation G236N, and the second Fc polypeptide of the heterodimeric Fc variant comprises a loop replacement and a mutation at position 236 selected from G236D, G236E, G236K, G236N and G236T.

In some embodiments, the heterodimeric Fc variant comprises a loop replacement in one Fc polypeptide and a mutation at position 236 in both Fc polypeptides, in which the mutation at position 236 is symmetric and is selected from G236D, G236N and G236K. In some embodiments, the heterodimeric Fc variant comprises a loop replacement in one Fc polypeptide and a mutation at position 236 in both Fc polypeptides, in which the mutation at position 236 is symmetric (i.e. the mutation at position 236 is the same in both Fc polypeptides) and is selected from G236D and G236N.

In some embodiments, the heterodimeric Fc variant comprises a loop replacement in one Fc polypeptide and an asymmetric mutation at position 236. In some embodiments, the first Fc polypeptide of the heterodimeric Fc variant comprises a mutation at position 236 selected from G236A, G236D, G236E, G236F, G236H, G236I, G236L, G236N, G236P, G236Q, G236S, G236T, G236V, G236W and G236Y, and the second Fc polypeptide of the heterodimeric Fc variant comprises a loop replacement and a mutation at position 236 selected from G236D, G236E, G236K, G236N and G236T, where the mutation at position 236 is asymmetric (i.e. the mutation at position 236 in the first Fc polypeptide is different to the mutation at position 236 in the second Fc polypeptide).

In some embodiments, the first Fc polypeptide of the heterodimeric Fc variant comprises a mutation at position 236 selected from G236A, G236E, G236F, G236H, G236I, G236L, G236N, G236P, G236Q, G236S, G236T, G236V, G236W and G236Y, and the second Fc polypeptide of the heterodimeric Fc variant comprises a loop replacement and the mutation G236D. In some embodiments, the first Fc polypeptide of the heterodimeric Fc variant comprises the mutation G236N, and the second Fc polypeptide of the heterodimeric Fc variant comprises a loop replacement and a mutation at position 236 selected from G236D, G236E, G236K and G236T.

In some embodiments, the first Fc polypeptide of the heterodimeric Fc variant comprises a mutation at position 236 selected from G236D, G236K and G236N, and the second Fc polypeptide of the heterodimeric Fc variant comprises a loop replacement and a mutation at position 236 selected from G236D and G236N, where the mutation at position 236 is asymmetric. In some embodiments, the first Fc polypeptide of the heterodimeric Fc variant comprises the mutation G236N and the second Fc polypeptide of the heterodimeric Fc variant comprises a loop replacement and the mutation G236D.

In certain embodiments, the heterodimeric Fc variant comprises a loop replacement in one Fc polypeptide, optionally a mutation at position 236 in one or both Fc polypeptides as described in any one of the embodiments above, and further comprises one or more “binding enhancers.”

A “binding enhancer” is an amino acid mutation known in the art or identified herein to increase the affinity of the Fc for FcγRIIb. Examples include, but are not limited to, L234F, L234W, L234D, L235F, L235W, G237F, G237A, G237L, S239D, S239E, V266I, V266L, S267A, S267E, S267I, S267Q, S267V, H268D, Y300E, K326D, K326E, K326N, I332L and I332E.

In certain embodiments, the heterodimeric Fc variant comprises one or more binding enhancer selected from L234F, L234W, L234D, L235F, L235W, G237F, G237A, G237L, S239D, S239E, V266I, V266L, S267A, S267E, S267I, S267Q, S267V, H268D, Y300E, K326D, K326E, K326N, I332L and I332E. In some embodiments, the heterodimeric Fc variant comprises one or more binding enhancer selected from S239D, S239E, V266I, V266L, S267A, S267E, S267I, S267Q, S267V, H268D, Y300E, K326D and I332E.

In certain embodiments, the heterodimeric Fc variant comprises a loop replacement in one Fc polypeptide, optionally a mutation at position 236 in one or both Fc polypeptides as described in any one of the embodiments above, and further comprises one or more binding enhancers selected from S239D, S239E, V266I, V266L, S267A, S267E, S267I, S267Q, S267V, H268D, Y300E, K326D and I332E. In some embodiments, the heterodimeric Fc variant comprises a loop replacement in one Fc polypeptide, optionally a mutation at position 236 in one or both Fc polypeptides as described in any one of the embodiments above, and further comprises one or more binding enhancers selected from S239D, S239E, V266I, V266L, S267A, S267I, S267V, S267Q and H268D. In some embodiments, the heterodimeric Fc variant comprises a loop replacement in one Fc polypeptide, optionally a mutation at position 236 in one or both Fc polypeptides as described in any one of the embodiments above, and further comprises one or more binding enhancers selected from S239D, S239E, V266L, S267A, S267I, S267V and H268D.

In certain embodiments, the heterodimeric Fc variant comprises a loop replacement in one Fc polypeptide, a mutation at position 236 in both Fc polypeptides as described in any one of the embodiments above, and further comprises one or more binding enhancers selected from S239D, S239E, V266I, V266L, S267A, S267E, S267I, S267Q, S267V, H268D, Y300E, K326D and I332E, where the one or more binding enhancers are located in the same Fc polypeptide as the loop replacement. In some embodiments, the heterodimeric Fc variant comprises a loop replacement in one Fc polypeptide, a mutation at position 236 in both Fc polypeptides as described in any one of the embodiments above, and further comprises one or more binding enhancers selected from S239D, S239E, V266I, V266L, S267A, S267I, S267V, S267Q and H268D, where the one or more binding enhancers are located in the same Fc polypeptide as the loop replacement. In some embodiments, the heterodimeric Fc variant comprises a loop replacement in one Fc polypeptide, a mutation at position 236 in both Fc polypeptides as described in any one of the embodiments above, and further comprises one or more binding enhancers selected from S239D, S239E, V266L, S267A, S267I, S267V and H268D, where the one or more binding enhancers are located in the same Fc polypeptide as the loop replacement.

In certain embodiments, the heterodimeric Fc variant comprises a loop replacement in one Fc polypeptide, an asymmetric mutation at position 236 in both Fc polypeptides as described in any one of the embodiments above, and further comprises one or more binding enhancers selected from S239D, S239E, V266I, V266L, S267A, S267E, S267I, S267Q, S267V, H268D, Y300E, K326D and I332E, where the one or more binding enhancers are located in the same Fc polypeptide as the loop replacement. In some embodiments, the heterodimeric Fc variant comprises a loop replacement in one Fc polypeptide, an asymmetric mutation at position 236 in both Fc polypeptides as described in any one of the embodiments above, and further comprises one or more binding enhancers selected from S239D, S239E, V266I, V266L, S267A, S267I, S267V, S267Q and H268D, where the one or more binding enhancers are located in the same Fc polypeptide as the loop replacement. In some embodiments, the heterodimeric Fc variant comprises a loop replacement in one Fc polypeptide, an asymmetric mutation at position 236 in both Fc polypeptides as described in any one of the embodiments above, and further comprises one or more binding enhancers selected from S239D, S239E, V266L, S267A, S267I, S267V and H268D, where the one or more binding enhancers are located in the same Fc polypeptide as the loop replacement.

In some embodiments, the first Fc polypeptide of the heterodimeric Fc variant comprises a mutation at position 236 selected from G236A, G236D, G236E, G236F, G236H, G236I, G236L, G236N, G236P, G236Q, G236S, G236T, G236V, G236W and G236Y, and the second Fc polypeptide of the heterodimeric Fc variant comprises a loop replacement, a mutation at position 236 selected from G236D, G236E, G236K, G236N and G236T, and one or more binding enhancers selected from S239D, S239E, V266I, V266L, S267A, S267I, S267V, S267Q and H268D. In some embodiments, the first Fc polypeptide of the heterodimeric Fc variant comprises a mutation at position 236 selected from G236A, G236D, G236E, G236F, G236H, G236I, G236L, G236N, G236P, G236Q, G236S, G236T, G236V, G236W and G236Y, and the second Fc polypeptide of the heterodimeric Fc variant comprises a loop replacement, the mutation G236D, and one or more binding enhancers selected from S239D, S239E, V266I, V266L, S267A, S267I, S267V, S267Q and H268D. In some embodiments, the first Fc polypeptide of the heterodimeric Fc variant comprises the mutation G236N, and the second Fc polypeptide of the heterodimeric Fc variant comprises a loop replacement, a mutation at position 236 selected from G236D, G236E, G236K, G236N and G236T, and one or more binding enhancers selected from S239D, S239E, V266I, V266L, S267A, S267I, S267V, S267Q and H268D.

In certain embodiments, the binding enhancers comprised by the heterodimeric Fc variant comprise (i) the mutation S239D or S239E, and/or (ii) the mutation H268D. In some embodiments, the binding enhancers comprised by the heterodimeric Fc variant comprise (i) the mutation S239D or S239E, and/or (ii) the mutation H268D, and/or (iii) the mutation S267A, S267I or S267V. In some embodiments, the binding enhancers comprised by the heterodimeric Fc variant comprise the mutations S239D and H268D. In some embodiments, the binding enhancers comprised by the heterodimeric Fc variant comprise the mutations S239D, H268D and S267V. In some embodiments, the binding enhancers comprise the mutations S239D, H268D and S267A.

In certain embodiments, the heterodimeric Fc variant comprises (a) a mutation at position 236 in one or both of the first and second Fc polypeptides as described in any one of the embodiments above, (b) a loop replacement in the second Fc polypeptide, (c) one or more “binding enhancers” in the second Fc polypeptide as described in any one of the embodiments above, (d) optionally additional CH2 mutations at one or more of positions 234, 235, 237 and 239 in the first Fc polypeptide, and (e) optionally additional CH2 mutations at one or more of positions 234, 235, 237, 240, 263, 264, 266, 269, 271, 273, 323 and 332 in the second Fc polypeptide.

In some embodiments, the additional CH2 mutations at one or more of positions 234, 235, 237 and 239 in the first Fc polypeptide of the heterodimeric Fc variant are selected from:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

In some embodiments, the additional CH2 mutations at one or more of positions 234, 235, 237 and 239 in the first Fc polypeptide of the heterodimeric Fc variant are selected from:
(i) the mutation at position 234 is selected from L234D, L234F, L234Q, L234T and L234W,
(ii) the mutation at position 235 is selected from L235A, L235D, L235E, L235F, L235H, L235R, L235W and L235Y,
(iii) the mutation at position 237 is selected from G237A, G237D, G237L and G237N, and
(iv) the mutation at position 239 is selected from S239A, S239G, S239H, S239T and S239Y.

In some embodiments, the first Fc polypeptide of the heterodimeric Fc polypeptide comprises additional CH2 mutations selected from L234D and L235F.

In some embodiments, the additional CH2 mutations at one or more of positions 234, 235, 237, 240, 263, 264, 266, 269, 271, 273, 323 and 332 in the second Fc polypeptide of the heterodimeric Fc variant are selected from:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

In some embodiments, the second Fc polypeptide of the heterodimeric Fc variant comprises additional CH2 mutations at one or more of positions 271, 323 and 332 selected from: (i) the mutation P271D, (ii) the mutation V323A, and (iii) a mutation at position 332 selected from I332F and I332L.

In certain embodiments, the heterodimeric Fc variant comprises the amino acid mutations as set out in Table 5A, Table 5B and Table 5C for any one of the variants listed under “Loop Replacement+Symmetrical 236 Mutation,” “Strategy 1/3” or “Strategy 1/3+Strategy 2 Combinations.” In certain embodiments, the heterodimeric Fc variant comprises the amino acid mutations as set out for any one of the variants shown in Table 6.22, 6.24, 6.25 and 6.27. In certain embodiments, the heterodimeric Fc variant comprises the amino acid mutations as set out for any one of the variants shown in Table 6.22 and 6.24.

In certain embodiments, the heterodimeric Fc variant comprises the amino acid mutations of any one of the variants shown in Tables 6.17, 6.19 and 6.20 having a “IIb Selectivity Fold wrt Control” value≥0.5 and a “Ib-Fold wrt Control” value≥0.5 (“Criteria B”). In some embodiments, the heterodimeric Fc variant comprises the amino acid mutations of any one of the variants shown in Tables 6.17, 6.19 and 6.20 having a “IIb Selectivity Fold wrt Control” value≥1.0 and a “IIb-Fold wrt Control” value≥0.3 (“Criteria C”). In some embodiments, the heterodimeric Fc variant comprises the amino acid mutations of any one of the variants shown in Tables 6.17, 6.19 and 6.20 having a “IIb Selectivity Fold wrt Control” value≥1.0 and a “IIb-Fold wrt Control” value≥0.5 (“Criteria D”). In some embodiments, the heterodimeric Fc variant comprises the amino acid mutations of any one of the variants shown in Tables 6.17, 6.19 and 6.20 having a “IIb Selectivity Fold wrt Control” value≥1.5 and a “IIb-Fold wrt Control” value≥0.3 (“Criteria A”).

### Heterodimeric Fc Variants Comprising an Asymmetric Mutation at Position 236

As described herein, incorporating an asymmetrical mutation at position 236 in the CH2 domain of the Fc has been found to increase selectivity for FcγRIIb. Accordingly, certain embodiments of the present disclosure relate to heterodimeric Fc variants that comprise an asymmetric mutation at position 236 and have increased selectivity for FcγRIIb as compared to the parental Fc. The asymmetric mutation at position 236 may comprise an amino acid mutation at position 236 in one Fc polypeptide and no mutation at position 236 in the other Fc polypeptide, or it may comprise a mutation at position 236 in one Fc polypeptide and a different mutation at position 236 in the other Fc polypeptide.

In certain embodiments in which the heterodimeric Fc variants comprise an asymmetric mutation at position 236 and have increased selectivity for FcγRIIb as compared to the parental Fc, the asymmetric mutation at position 236 comprises a mutation selected from G236N and G236D. In some embodiments, the heterodimeric Fc variant comprises an asymmetric mutation at position 236 in which one Fc polypeptide comprises the mutation G236N or G236D, and the other Fc polypeptide does not comprise a mutation at position 236. In some embodiments, the heterodimeric Fc variant comprises an asymmetric mutation at position 236 in which one Fc polypeptide comprises the mutation G236N or G236D, and the other Fc polypeptide comprises a different mutation at position 236. In some embodiments, the heterodimeric Fc variant comprises an asymmetric mutation at position 236 in which one Fc polypeptide comprises the mutation G236N, and the other Fc polypeptide comprises the mutation G236D.

In certain embodiments, the heterodimeric Fc variant comprises an asymmetric mutation at position 236 in which one Fc polypeptide comprises the mutation G236N, and the other Fc polypeptide comprises the mutation G236D, G236K or G236S, or does not include a mutation at position 236.

In certain embodiments, the heterodimeric Fc variant comprises an asymmetric mutation at position 236 in which one Fc polypeptide comprises the mutation G236D, and the other Fc polypeptide comprises the mutation G236N, G236Q, G236K, G236E or G236H, or does not include a mutation at position 236.

In certain embodiments, the heterodimeric Fc variant comprises an asymmetric mutation at position 236 as described in any one of the embodiments above and one or more additional mutations in the CH2 domain. The one or more additional mutations in the CH2 domain may be symmetric mutations or asymmetric mutations and may increase the selectivity of the heterodimeric Fc variant for FcγRIIb, or increase the affinity of the heterodimeric Fc variant for FcγRIIb, or increase both the selectivity and affinity of the heterodimeric Fc variant for FcγRIIb. In some embodiments, the heterodimeric Fc variant comprises an asymmetric mutation at position 236 as described in any one of the embodiments above and one or more additional asymmetric mutations in the CH2 domain.

In certain embodiments, the heterodimeric Fc variant comprises between one and 20 mutations in the CH2 domain, including an asymmetric mutation at position 236. In some embodiments, the heterodimeric Fc variant comprises an asymmetric mutation at position 236 and between one and 18 additional mutations in the CH2 domain, for example, between one and 17 additional mutations, between one and 16 additional mutations, or between one and 15 additional mutations in the CH2 domain.

### Binding Enhancers

In certain embodiments, the heterodimeric Fc variant comprises an asymmetric mutation at position 236 as described in any one of the embodiments above, and further comprises one or more “binding enhancers” as described above. In some embodiments, the one or more binding enhancers are selected from S239D, S239E, V266I, V266L, S267A, S267I, S267V, S267Q and H268D. In some embodiments, the one or more binding enhancers are selected from S239D, S239E, V266L, S267A, S267I, S267V and H268D.

In some embodiments, the heterodimeric Fc variant comprises an asymmetric mutation at position 236 selected from G236N and G236D and further comprises one or more binding enhancers as described above. In some embodiments, the heterodimeric Fc variant comprises an asymmetric mutation at position 236 in which the first Fc polypeptide comprises the mutation G236N or G236D, and the second Fc polypeptide does not comprise a mutation at position 236, and in which the second Fc polypeptide further comprises one or more binding enhancers as described above. In some embodiments, the heterodimeric Fc variant comprises an asymmetric mutation at position 236 in which the first Fc polypeptide comprises the mutation G236N or G236D, and the second Fc polypeptide comprises a different mutation at position 236, and in which the second Fc polypeptide further comprises one or more binding enhancers as described above. In some embodiments, the one or more binding enhancers are selected from S239D, S239E, V266L, S267A, S267I, S267V and H268D.

In certain embodiments, the heterodimeric Fc variant comprises an asymmetric mutation at position 236 in which the first Fc polypeptide comprises the mutation G236N, and the second Fc polypeptide comprises the mutation G236D, G236K or G236S, and the second Fc polypeptide further comprises one or more binding enhancers as described above. In some embodiments, the one or more binding enhancers are selected from S239D, S239E, V266L, S267A, S267I, S267V and H268D.

In certain embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises the binding enhancers (i) S239D or S239E, and/or (ii) H268D. In some embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises the mutations S239D and H268D.

In certain embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises the binding enhancers (i) S239D or S239E, and/or (ii) H268D, and/or (iii) S267A, S267I or S267V. In some embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises the mutations S239D, H268D and S267V. In some embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises the mutations S239D, H268D and S267A.

In certain embodiments, the heterodimeric Fc variant comprises an asymmetric mutation at position 236 in which the first Fc polypeptide comprises the mutation G236N, and the second Fc polypeptide comprises the mutation G236D, and the second Fc polypeptide further comprises one or more binding enhancers as described above. In some embodiments, the one or more binding enhancers are selected from S239D, S239E, V266L, S267A, S267I, S267V and H268D.

In certain embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the binding enhancers (i) S239D or S239E, and/or (ii) H268D. In some embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the mutations S239D and H268D.

In certain embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the binding enhancers (i) S239D or S239E, and/or (ii) H268D, and/or (iii) S267A, S267I or S267V. In some embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the mutations S239D, H268D and S267V. In some embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the mutations S239D, H268D and S267A. In some embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises the mutations S239D, H268D and S267I.

In certain embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises one or more binding enhancers as described above. In some embodiments, the one or more binding enhancers are selected from S239D, S239E, V266L, S267A, S267I, S267V and H268D.

In some embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises the binding enhancers (i) S239D or S239E, and/or (ii) H268D. In some embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises the mutations S239D and H268D.

In some embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises the binding enhancers (i) S239D or S239E, and/or (ii) H268D, and/or (iii) S267A, S267I or S267V. In some embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises the mutations S239D, H268D and S267V.

In certain embodiments, the heterodimeric Fc variant comprises the amino acid mutations as set out in Core Set 1 below:

### Core Set 1

First Fc polypeptide: G236N
Second Fc polypeptide: G236D_S239D_H268D.

In certain embodiments, the heterodimeric Fc variant comprises the amino acid mutations as set out in Core Set 1A below:

### Core Set 1A

First Fc polypeptide: G236N
Second Fc polypeptide: G236D_S239D_S267A/I/V_H268D.

In certain embodiments, the heterodimeric Fc variant comprises the amino acid mutations as set out in Table 5A for any one of the variants listed under “Asymmetric 236 Mutation.”

### Additional CH2 Domain Mutations

As described in the Examples provided herein, various in silico approaches were employed to identify Fc variants having increased selectivity for FcγRIIb. Experimental testing and refinement of the initially identified variants led to the identification of two lead variants having increased selectivity for FcγRIIb, Lead 1 and Lead 2 (see Example 3 and Table 4), each of which included an asymmetric mutation at position 236, and one or more binding enhancers, together with additional CH2 domain mutations. Further refinement of these Lead variants (see Example 4) produced Launching Modules 1 and 2 (see Table 4), each of which also included an asymmetric mutation at position 236, one or more binding enhancers and additional CH2 domain mutations. Additional rounds of investigation based on Launching Modules 1 and 2 identified alternative amino acid substitutions that could be made at the CH2 domain positions mutated in these Launching Modules, as well as additional CH2 domain mutations that could be included in the heterodimeric Fc variant to further improve FcγRIIb selectivity and/or affinity (see Example 6). Certain embodiments of the present disclosure thus relate to heterodimeric Fc variants comprising included an asymmetric mutation at position 236, one or more binding enhancers and one or more additional CH2 domain mutations.

TABLE 4
Initial FcγRIIb Selective Variants

| | CH2 Domain Mutations | |
|---|---|---|
| | Chain A | Chain B |
| Lead 1 | L234D_G236N | Template 1 (replacement loop) + |
| (v19544) | | G236D_S239D_S267I_H268D |
| Launching | G236N_G237A | Template 1 (replacement loop) + |
| Module 1 | | G236D_G237F_S239D_S267V_{—} |
| (v27293) | | H268D |
| Lead 2 | L234F_G236N_{—} | L234F_G236D_S239D_V266L_{—} |
| (v19585) | H268Q_K274Q_{—} | S267A_H268D_K274Q_A327G_{—} |
| | A327G_A330K_{—} | A330S_P331S |
| | P331S | |
| Launching | L234F_G236N_{—} | G236D_S239D_V266L_S267A_{—} |
| Module 2 | H268Q_A327G_{—} | H268D |
| (v27294) | A330K_P331S | |

### Strategy 1/3 Variants

Further optimization of Launching Module 1 was undertaken providing additional heterodimeric Fc variants having improved selectivity for FcγRIIb, which are collectively referred to in the following sections as “Strategy 1/3 variants.” The term “Strategy 1/3 variants” as used herein refers to those heterodimeric Fc variants that comprise: (a) an asymmetric mutation at position 236 as described above, (b) an asymmetric loop replacement in the CH2 domain, (c) optionally one or more binding enhancers as described above, and (d) optionally one or more additional mutations in the CH2 domain. As such, the term is not limited to the heterodimeric Fc variants explicitly referred to in the Examples as “Strategy 1 variants” and “Strategy 3 variants.” In certain embodiments, a Strategy 1/3 variant is a heterodimeric Fc variants that comprises: (a) an asymmetric mutation at position 236 as described above, (b) an asymmetric loop replacement in the CH2 domain, (c) one or more binding enhancers as described above, and (d) optionally one or more additional mutations in the CH2 domain.

In certain embodiments, the heterodimeric Fc variant is a Strategy 1/3 variant. In some embodiments, the heterodimeric Fc variant comprises an asymmetric mutation at position 236 as described in any one of the embodiments above and further comprises an asymmetric loop replacement in the CH2 domain. In some embodiments, the asymmetric loop replacement comprised by the heterodimeric Fc variant comprises replacement of the native loop at positions 325 to 331 in one Fc polypeptide with a polypeptide loop of between 7 and 15 amino acids in length or between 8 and 15 amino acids in length as described in any one of the embodiments provided above under “Asymmetric Loop Replacement.”

In certain embodiments, the heterodimeric Fc variant comprises an asymmetric mutation at position 236 selected from G236N and G236D, and further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.” In some embodiments, the heterodimeric Fc variant comprises an asymmetric mutation at position 236 in which the first Fc polypeptide comprises the mutation G236N or G236D, and the second Fc polypeptide does not comprise a mutation at position 236, and in which the second Fc polypeptide further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.” In some embodiments, the heterodimeric Fc variant comprises an asymmetric mutation at position 236 in which the first Fc polypeptide comprises the mutation G236N or G236D, and the second Fc polypeptide comprises a different mutation at position 236 and further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.”

In certain embodiments, the heterodimeric Fc variant comprises an asymmetric mutation at position 236 in which the first Fc polypeptide comprises the mutation G236N, and the second Fc polypeptide comprises the mutation G236D, G236K or G236S, and in which the second Fc polypeptide further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.” In some embodiments, the heterodimeric Fc variant comprises an asymmetric mutation at position 236 in which the first Fc polypeptide comprises the mutation G236N, and the second Fc polypeptide comprises the mutation G236D, and in which the second Fc polypeptide further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.” In some embodiments, the heterodimeric Fc variant comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises replacement of the native loop at positions 325 to 331 with a polypeptide loop of between 7 and 15 amino acids in length or between 8 and 15 amino acids in length as described in any one of the embodiments provided above under “Asymmetric Loop Replacement.”

In certain embodiments, the heterodimeric Fc variant is a Strategy 1/3 variant and comprises the following amino acid mutations (referred to as Core Set 2):

### Core Set 2

First Fc polypeptide: G236N
Second Fc polypeptide: G236D_Loop Replacement (325-331).

In certain embodiments, the replacement loop comprised by the Strategy 1/3 variant is a polypeptide loop comprising an amino acid sequence as set forth in any one of Formula (I), Formula (Ia), Formula (Ib), Formula (II), Formula (ITT), Formula (IV), Formula (V) or Formula (VI), as described above under “Asymmetric Loop Replacement.” In some embodiments, the polypeptide loop comprises an amino acid sequence as set forth in any one of the sequences shown in Tables 3A and 3B (SEQ ID NOs: 4-172). In some embodiments, the polypeptide loop comprises an amino acid sequence as set forth in any one of SEQ ID NOs: 4-90 (see Table 3A above). In some embodiments, the polypeptide loop comprises an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 (see Table 3A above).

In certain embodiments, the heterodimeric Fc variant is a Strategy 1/3 variant comprising the amino acid mutations set out in Core Set 2 and in which the second Fc polypeptide further comprises: (a) an amino acid mutation at position 239 selected from S239D and S239E, (b) an amino acid mutation at position 267 selected from S267I, S267Q and S267V, and (c) an amino acid mutation at position 268 selected from H268A, H268D, H268E, H268F, H268I, H268K, H268L, H268N, H268P, H268Q, H268T, H268V, H268W and H268Y.

In certain embodiments, the heterodimeric Fc variant is a Strategy 1/3 variant and comprises: (a) an asymmetric mutation at position 236 as described in any one of the embodiments above, (b) replacement of the native loop at positions 325 to 331 in one Fc polypeptide with a polypeptide loop of between 7 and 15 amino acids in length or between 8 and 15 amino acids in length as described in any one of the embodiments provided above under “Asymmetric Loop Replacement,” and (c) one or more binding enhancers as described in any one of the embodiments above.

In certain embodiments, the heterodimeric Fc variant is a Strategy 1/3 variant and comprises: (a) an asymmetric mutation at position 236 in which the first Fc polypeptide comprises the mutation G236N and the second Fc polypeptide comprises the mutation G236D, G236K or G236S, (b) replacement of the native loop at positions 325 to 331 in the second Fc polypeptide with a polypeptide loop of between 7 and 15 amino acids in length or between 8 and 15 amino acids in length as described in any one of the embodiments provided above under “Asymmetric Loop Replacement,” and (c) one or more binding enhancers in the second Fc polypeptide as described in any one of the embodiments above.

In certain embodiments, the heterodimeric Fc variant is a Strategy 1/3 variant and comprises: (a) an asymmetric mutation at position 236 in which the first Fc polypeptide comprises the mutation G236N and the second Fc polypeptide comprises the mutation G236D, (b) replacement of the native loop at positions 325 to 331 in the second Fc polypeptide with a polypeptide loop of between 7 and 15 amino acids in length or between 8 and 15 amino acids in length as described in any one of the embodiments provided above under “Asymmetric Loop Replacement,” and (c) one or more binding enhancers in the second Fc polypeptide as described in any one of the embodiments above.

In certain embodiments, the heterodimeric Fc variant is a Strategy 1/3 variant and comprises: (a) an asymmetric mutation at position 236 in which the first Fc polypeptide comprises the mutation G236D and the second Fc polypeptide comprises the mutation G236N, G236Q, G236K, G236E or G236H, (b) replacement of the native loop at positions 325 to 331 in the second Fc polypeptide with a polypeptide loop of between 7 and 15 amino acids in length or between 8 and 15 amino acids in length as described in any one of the embodiments provided above under “Asymmetric Loop Replacement,” and (c) one or more binding enhancers in the second Fc polypeptide as described in any one of the embodiments above.

In certain embodiments, the heterodimeric Fc variant is a Strategy 1/3 variant comprising the amino acid mutations set out as Core Set 2, and the second Fc polypeptide further comprises one or more binding enhancers.

In certain embodiments, the one or more binding enhancers included in the Strategy 1/3 heterodimeric Fc variant are selected from S239D, S239E, V266I, S267I, S267Q, S267V and H268D. In some embodiments, the one or more binding enhancers are (i) S239D or S239E, and/or (ii) H268D, and/or (iii) S267I or S267V. In some embodiments, the one or more binding enhancers are S239D and H268D. In some embodiments, the one or more binding enhancers are S239D, H268D and S267V.

In some embodiments, the heterodimeric Fc variant is a Strategy 1/3 variant and comprises the following amino acid mutations (referred to as Core Set 2A):

### Core Set 2A

First Fc polypeptide: G236N
Second Fc polypeptide: G236D_S239D_H268D_Loop Replacement (325-331).

In some embodiments, the heterodimeric Fc variant is a Strategy 1/3 variant and comprises the following amino acid mutations (referred to as Core Set 2B):

### Core Set 2B

First Fc polypeptide: G236N
Second Fc polypeptide: G236D_S239D_S267I/V_H268D_Loop Replacement (325-331).

In certain embodiments, the heterodimeric Fc variant is a Strategy 1/3 variant comprising the amino acid mutations as set out in Core Set 2A in which the asymmetric mutation at position 236 has been modified as shown in Core Set 2C and Core Set 2D below.

### Core Set 2C

First Fc polypeptide: G236N
Second Fc polypeptide: G236D, E, K or T
   +S239D_H268D_Loop Replacement (325-331).

### Core Set 2D

First Fc polypeptide: G236N, A, E, F, H, I, L, P, Q, S, T, V, W or Y, or no G236 mutation
Second Fc polypeptide: G236D_S239D_H268D_Loop Replacement (325-331).

In some embodiments, the heterodimeric Fc variant comprises the amino acid mutations set out in Core Set 2C in which the second Fc polypeptide comprises the mutation G236D or G236K.

In certain embodiments, the heterodimeric Fc variant is a Strategy 1/3 variant comprising the amino acid mutations as set out in Core Set 2B in which the asymmetric mutation at position 236 has been modified as shown in Core Set 2E and Core Set 2F below.

### Core Set 2E

First Fc polypeptide: G236N
Second Fc polypeptide: G236D, E, K or T
   +S239D_S267I/V_H268D_Loop Replacement (325-331).

### Core Set 2F

First Fc polypeptide: G236N, A, E, F, H, I, L, P, Q, S, T, V, W or Y, or no G236 mutation
Second Fc polypeptide: G236D_S239D_S267I/V_H268D_Loop Replacement (325-331).

In some embodiments, the heterodimeric Fc variant comprises the amino acid mutations set out in Core Set 2E in which the second Fc polypeptide comprises the mutation G236D or G236K.

Introducing an aspartate (D) or asparagine (N) residue at position 236 in the heterodimeric Fc variant may potentially introduce a deamidation site into the Fc as the G236D/N mutation would precede the natural glycine (G) residue at position 237. Accordingly, in certain embodiments in which the heterodimeric Fc variant comprises the mutation G236D and/or the mutation G236N, the heterodimeric Fc variant may optionally further comprise an amino acid mutation at position G237.

In some embodiments in which the heterodimeric Fc variant is a Strategy 1/3 variant and comprises the mutation G236D in one Fc polypeptide, the same Fc polypeptide may further comprise an amino acid mutation at position G237 selected from G237F, G237I, G237K, G237L, G237Q, G237T, G237V and G237Y. In some embodiments in which the heterodimeric Fc variant comprises the mutation G236D in one Fc polypeptide, the same Fc polypeptide may further comprise the amino acid mutation G237F.

In some embodiments in which the heterodimeric Fc variant is a Strategy 1/3 variant and comprises the mutation G236N in one Fc polypeptide, the same Fc polypeptide may further comprise an amino acid mutation at position G237 selected from G237A, G237D, G237F, G237H, G237L, G237N, G237P, G237S, G237V, G237W and G237Y. In some embodiments in which the heterodimeric Fc variant comprises the mutation G236N in one Fc polypeptide, the same Fc polypeptide may further comprise the amino acid mutation G237A.

In certain embodiments in which the heterodimeric Fc variant is a Strategy 1/3 variant comprising the mutation G236N in the first Fc polypeptide, the first Fc polypeptide may further comprise additional CH2 mutations at one or more of positions 234, 235, 237 and 239.

In some embodiments, the heterodimeric Fc variant is a Strategy 1/3 variant which comprises the amino acid mutations as set out in any one of Core Sets 2, 2A, 2B, 2C, 2D, 2E or 2F, and the first Fc polypeptide may further comprise additional CH2 mutations at one or more of positions 234, 235, 237 and 239.

In some embodiments in which the first Fc polypeptide further comprises additional CH2 mutations at one or more of positions 234, 235, 237 and 239:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

In some embodiments the first Fc polypeptide further comprises additional CH2 mutations at one or more of positions 234, 235, 237 and 239:
(i) the mutation at position 234 is selected from L234D, L234F, L234Q, L234T and L234W,
(ii) the mutation at position 235 is selected from L235A, L235D, L235E, L235F, L235H, L235R, L235W and L235Y,
(iii) the mutation at position 237 is selected from G237A, G237D, G237L and G237N, and
(iv) the mutation at position 239 is selected from S239A, S239G, S239H, S239T and S239Y.

In some embodiments, the heterodimeric Fc polypeptide is a Strategy 1/3 variant which comprises the mutation G236N in the first Fc polypeptide and the first Fc polypeptide further comprises the mutation L234D.

In some embodiments, the heterodimeric Fc variant is a Strategy 1/3 variant which comprises the amino acid mutations as set out in any one of Core Sets 2, 2A, 2B, 2C, 2D, 2E or 2F, and the first Fc polypeptide further comprises the mutation L234D.

In some embodiments, the heterodimeric Fc polypeptide is a Strategy 1/3 variant which comprises the mutation G236N in the first Fc polypeptide, and the first Fc polypeptide further comprises the mutation L235F.

In some embodiments, the heterodimeric Fc variant is a Strategy 1/3 variant which comprises the amino acid mutations as set out in any one of Core Sets 2, 2A, 2B, 2C, 2D, 2E or 2F, and the first Fc polypeptide further comprises the mutation L235F.

In certain embodiments, the heterodimeric Fc variant is a Strategy 1/3 variant which comprises the mutation G236D and replacement of the loop at positions 325-331 in the second Fc polypeptide, and the second Fc polypeptide may further comprise additional CH2 mutations at one or more of positions 234, 235, 237, 240, 263, 264, 266, 269, 271, 273, 323 and 332.

In some embodiments, the heterodimeric Fc variant is a Strategy 1/3 variant which comprises the amino acid mutations as set out in any one of Core Sets 2, 2A, 2B, 2C, 2D, 2E or 2F, and the second Fc polypeptide may further comprise additional CH2 mutations at one or more of positions 234, 235, 237, 240, 263, 264, 266, 269, 271, 273, 323 and 332.

In some embodiments in which the second Fc polypeptide further comprises additional CH2 mutations at one or more of positions 234, 235, 237, 240, 263, 264, 266, 269, 271, 273, 323 and 332:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

In some embodiments in which the second Fc polypeptide further comprises additional CH2 mutations at one or more of positions 271, 323 and 332:
(i) the mutation at position 271 is P271D,
(ii) the mutation at position 323 is V323A, and
(iii) the mutation at position 332 is selected from I332F and I332L.

In certain embodiments, the heterodimeric Fc variant is a Strategy 1/3 variant and comprises the amino acid mutations as set out in Table 5A, Table 5B and Table 5C for any one of the variants listed under “Strategy 1/3” and “Strategy 1/3+Strategy 2 Combinations.” In certain embodiments, the heterodimeric Fc variant is a Strategy 1/3 variant and comprises the amino acid mutations as set out for any one of the variants shown in Tables 6.22, 6.24, 6.25 and 6.27. In some embodiments, the heterodimeric Fc variant is a Strategy 1/3 variant and comprises the amino acid mutations as set out for any one of the variants shown in Tables 6.22 and 6.24.

In certain embodiments, the heterodimeric Fc variant is a Strategy 1/3 variant and comprises the amino acid mutations of any one of the variants shown in Tables 6.17, 6.19 and 6.20 that has a “IIb Selectivity Fold wrt Control” value≥0.5 and a “IIb-Fold wrt Control” value≥0.5 (“Criteria B”). In some embodiments, the heterodimeric Fc variant comprises the amino acid mutations of any one of the variants shown in Tables 6.17, 6.19 and 6.20 that has a “IIb Selectivity Fold wrt Control” value≥1.0 and a “IIb-Fold wrt Control” value≥0.3 (“Criteria C”). In certain embodiments, the heterodimeric Fc variant comprises the amino acid mutations of any one of the variants shown in Tables 6.17, 6.19 and 6.20 that has a “IIb Selectivity Fold wrt Control” value≥1.0 and a “IIb-Fold wrt Control” value≥0.5 (“Criteria D”). In certain embodiments, the heterodimeric Fc variant comprises the amino acid mutations of any one of the variants shown in Tables 6.17, 6.19 and 6.20 that has a “IIb Selectivity Fold wrt Control” value≥1.5 and a “IIb-Fold wrt Control” value≥0.3 (“Criteria A”).

### Strategy 2 Variants

Further optimization of Launching Module 2 was undertaken providing additional heterodimeric Fc variants having improved selectivity for FcγRIIb, which are referred to herein as “Strategy 2 variants.” The term “Strategy 2 variants” as used herein refers to those heterodimeric Fc variants that comprise: (a) an asymmetric mutation at position 236 as described above, (b) one or more binding enhancers as described above, (c) one or more IgG4-based mutations, and (d) optionally one or more additional mutations in the CH2 domain. As such, this term is not limited to describing those heterodimeric Fc variants explicitly referred to in the Examples as “Strategy 2 variants.”

In certain embodiments, the heterodimeric Fc variant is a Strategy 2 variant. In certain embodiments, the heterodimeric Fc variant comprises an asymmetric mutation at position 236 as described in any one of the embodiments above, one or more binding enhancers as described in any one of the embodiments above, and a mutation at one or more positions selected from 234, 268, 327, 330 and 331. In some embodiments, the heterodimeric Fc variant comprises an asymmetric mutation at position 236 as described in any one of the embodiments above, one or more binding enhancers as described in any one of the embodiments above in one Fc polypeptide, and a mutation at one or more positions selected from 234, 268, 327, 330 and 331 in the other Fc polypeptide.

In certain embodiments, the heterodimeric Fc variant is a Strategy 2 variant and comprises an asymmetric mutation at position 236 as described in any one of the embodiments above, one or more binding enhancers in one Fc polypeptide selected from S239D, S239E, V266L, S267A, S267I, S267Q, S267V and H268D, and a mutation at one or more positions selected from 234, 268, 327, 330 and 331 in the other Fc polypeptide.

In certain embodiments, the heterodimeric Fc variant is a Strategy 2 variant and comprises an asymmetric mutation at position 236 selected from G236N and G236D, one or more binding enhancers in one Fc polypeptide selected from S239D, S239E, V266L, S267A, S267I, S267Q, S267V and H268D, and a mutation at one or more positions selected from 234, 268, 327, 330 and 331 in the other Fc polypeptide.

In certain embodiments, the heterodimeric Fc variant is a Strategy 2 variant and comprises an asymmetric mutation at position 236 in which the first Fc polypeptide comprises the mutation G236N, and the second Fc polypeptide comprises the mutation G236D, G236K or G236S, and in which the second Fc polypeptide further comprises one or more binding enhancers selected from S239D, S239E, V266L, S267A, S267I, S267Q, S267V and H268D, and the first Fc polypeptide further comprises a mutation at one or more positions selected from 234, 268, 327, 330 and 331.

In certain embodiments, the heterodimeric Fc variant is a Strategy 2 variant and comprises an asymmetric mutation at position 236 in which the first Fc polypeptide comprises the mutation G236N, and the second Fc polypeptide comprises the mutation G236D, and in which the second Fc polypeptide further comprises one or more binding enhancers selected from S239D, S239E, V266L, S267A, S267I, S267Q, S267V and H268D, and the first Fc polypeptide further comprises a mutation at one or more positions selected from 234, 268, 327, 330 and 331.

In certain embodiments, the heterodimeric Fc variant is a Strategy 2 variant and comprises an asymmetric mutation at position 236 in which the first Fc polypeptide comprises the mutation G236N, and the second Fc polypeptide comprises the mutation G236D, and in which the second Fc polypeptide further comprises one or more binding enhancers selected from S239D, S239E, V266L, S267A, S267I, S267Q and S267V and a mutation at position 268 selected from H268A, H268D, H268E, H268F, H268N, H268Q, H268S, H268V, H268W and H268Y, and the first Fc polypeptide further comprises a mutation at one or more positions selected from 234, 268, 327, 330 and 331.

In certain embodiments, the heterodimeric Fc variant is a Strategy 2 variant and comprises an asymmetric mutation at position 236 in which the first Fc polypeptide comprises the mutation G236D, and the second Fc polypeptide comprises the mutation G236N, G236Q, G236K, G236E or G236H, and in which the second Fc polypeptide further comprises one or more binding enhancers selected from S239D, S239E, V266L, S267A, S267I, S267Q, S267V and H268D, and the first Fc polypeptide further comprises a mutation at one or more positions selected from 234, 268, 327, 330 and 331.

In some embodiments, the heterodimeric Fc variant is a Strategy 2 variant and comprises the amino acid mutations of Core Set 1, as described above:

### Core Set 1

First Fc polypeptide: G236N
Second Fc polypeptide: G236D_S239D_H268D,
in which the first Fc polypeptide further comprises a mutation at one or more positions selected from 234, 268, 327, 330 and 331.

In some embodiments, the heterodimeric Fc variant is a Strategy 2 variant and comprises the amino acid mutations of Core Set 1, in which the first Fc polypeptide further comprises a mutation at one or more positions selected from 234, 268, 327, 330 and 331, and the second Fc polypeptide further comprises the amino acid mutation S267A or S267Q.

In certain embodiments, the one or more binding enhancers included in the Strategy 2 heterodimeric Fc variant are selected from S239D, V266L, S267A, S267Q and H268D. In some embodiments, the one or more binding enhancers comprise the mutations S239D and/or H268D. In some embodiments, the one or more binding enhancers comprise the mutations S239D and H268D. In some embodiments, the one or more binding enhancers comprise the mutations S239D, H268D and (i) the mutation V266L, or (ii) the mutation S267A/Q, or (iii) the mutations V266L and S267A/Q. In some embodiments, the one or more binding enhancers comprise the mutations S239D, H268D, V266L and S267A. In some embodiments, the one or more binding enhancers comprise the mutations S239D, H268D, V266L and S267Q.

In certain embodiments, the mutation at one or more positions selected from 234, 268, 327, 330 and 331 comprised by the first Fc polypeptide of the Strategy 2 variant is one or more of:
(i) a mutation at position 234 selected from L234A, L234F, L234G, L234H, L234I, L234N, L234P, L234Q, L234S, L234T, L234V, L234W and L234Y,
(ii) a mutation at position 268 selected from H268A, H268D, H268E, H268F, H268G, H268I, H268K, H268L, H268N, H268P, H268Q, H268R, H268S, H268T, H268V, H268W and H268Y,
(iii) a mutation at position 327 selected from A327E and A327G;
(iv) a mutation at position 330 selected from A330K, A330H, A330Q, A330R, A330S and A330T, and
(v) a mutation at position 331 selected from P331A, P331D, P331E, P331H, P331Q and P331S.

In certain embodiments, the heterodimeric Fc variant is a Strategy 2 variant comprising an asymmetric mutation at position 236 as described in any one of the embodiments above, in which one Fc polypeptide comprises one or more binding enhancers selected from S239D, S239E, V266L, S267A, S267I, S267Q, S267V and H268D, and the other Fc polypeptide comprises a mutation at position 234 selected from L234A, L234F, L234G, L234H, L234I, L234N, L234P, L234Q, L234S, L234T, L234V, L234W and L234Y, and optionally a mutation at one or more of positions 268, 327, 330 and 331. In some embodiments, the one or more binding enhancers are S239D, H268D and optionally (i) V266L, or (ii) S267A/Q, or (iii) V266L and S267A/Q. In some embodiments, the mutation at position 234 is L234F.

In certain embodiments, the heterodimeric Fc variant is a Strategy 2 variant comprising an asymmetric mutation at position 236 as described in any one of the embodiments above, in which one Fc polypeptide comprises one or more binding enhancers selected from S239D, S239E, V266L, S267A, S267I, S267Q, S267V and H268D, and the other Fc polypeptide comprises a mutation at position 268 selected from H268A, H268D, H268E, H268F, H268G, H268I, H268K, H268L, H268N, H268P, H268Q, H268R, H268S, H268T, H268V, H268W and H268Y, and optionally a mutation at one or more of positions 234, 327, 330 and 331. In some embodiments, the one or more binding enhancers are S239D, H268D and optionally (i) V266L, or (ii) S267A/Q, or (iii) V266L and S267A/Q. In some embodiments, the mutation at position 268 is H268Q.

In some embodiments, the heterodimeric Fc variant is a Strategy 2 variant comprising an asymmetric mutation at position 236 as described in any one of the embodiments above, in which one Fc polypeptide comprises one or more binding enhancers selected from S239D, S239E, V266L, S267A, S267I, S267Q, S267V and H268D, and the other Fc polypeptide comprises a mutation at position 327 selected from A327E and A327G, and optionally a mutation at one or more of positions 234, 268, 330 and 331. In some embodiments, the one or more binding enhancers are S239D, H268D and optionally (i) V266L, or (ii) S267A/Q, or (iii) V266L and S267A/Q. In some embodiments, the mutation at position 327 is A327G.

In some embodiments, the heterodimeric Fc variant is a Strategy 2 variant comprising an asymmetric mutation at position 236 as described in any one of the embodiments above, in which one Fc polypeptide comprises one or more binding enhancers selected from S239D, S239E, V266L, S267A, S267I, S267Q, S267V and H268D, and the other Fc polypeptide comprises a mutation at position 330 selected from A330K, A330H, A330Q, A330R, A330S and A330T, and optionally a mutation at one or more of positions 234, 268, 327 and 331. In some embodiments, the one or more binding enhancers are S239D, H268D and optionally (i) V266L, or (ii) S267A/Q, or (iii) V266L and S267A/Q. In some embodiments, the mutation at position 330 is A330K or A330T. In some embodiments, the mutation at position 330 is A330K.

In some embodiments, the heterodimeric Fc variant is a Strategy 2 variant comprising an asymmetric mutation at position 236 as described in any one of the embodiments above, one Fc polypeptide comprises one or more binding enhancers selected from S239D, S239E, V266L, S267A, S267I, S267Q, S267V and H268D, and the other Fc polypeptide comprises a mutation at position 331 selected from P331A, P331D, P331E, P331H, P331Q and P331S, and optionally a mutation at one or more of positions 234, 268, 327 and 330. In some embodiments, the one or more binding enhancers are S239D, H268D and optionally (i) V266L, or (ii) S267A/Q, or (iii) V266L and S267A/Q. In some embodiments, the mutation at position 331 is P331S.

In certain embodiments, the heterodimeric Fc variant is a Strategy 2 variant and comprises an asymmetric mutation at position 236 in which the first Fc polypeptide comprises the mutation G236N and the second Fc polypeptide comprises the mutation G236D, and in which the second Fc polypeptide further comprises the binding enhancers S239D, H268D and optionally (i) V266L, or
(ii) S267A/Q, or (iii) V266L and S267A/Q, and the first Fc polypeptide further comprises one or more mutations selected from:
(i) a mutation at position 234 selected from L234A, L234F, L234G, L234H, L234I, L234N, L234P, L234Q, L234S, L234T, L234V, L234W and L234Y,
(ii) a mutation at position 268 selected from H268A, H268D, H268E, H268F, H268G, H268I, H268K, H268L, H268N, H268P, H268Q, H268R, H268S, H268T, H268V, H268W and H268Y,
(iii) a mutation at position 327 selected from A327E and A327G;
(iv) a mutation at position 330 selected from A330K, A330H, A330Q, A330R, A330S and A330T, and
(v) a mutation at position 331 selected from P331A, P331D, P331E, P331H, P331Q and P331S.

In certain embodiments, the heterodimeric Fc variant is a Strategy 2 variant and comprises an asymmetric mutation at position 236 in which the first Fc polypeptide comprises the mutation G236N and the second Fc polypeptide comprises the mutation G236D, and in which the second Fc polypeptide further comprises the binding enhancers S239D, H268D and optionally (i) V266L, or
(ii) S267A/Q, or (iii) V266L and S267A/Q, and the first Fc polypeptide further comprises the following mutations:
(i) a mutation at position 234 selected from L234A, L234F, L234G, L234H, L234I, L234N, L234P, L234Q, L234S, L234T, L234V, L234W and L234Y,
(ii) a mutation at position 268 selected from H268A, H268D, H268E, H268F, H268G, H268I, H268K, H268L, H268N, H268P, H268Q, H268R, H268S, H268T, H268V, H268W and H268Y,
(iii) a mutation at position 327 selected from A327G and A327E;
(iv) a mutation at position 330 selected from A330K, A330H, A330Q, A330R, A330S and A330T, and
(v) a mutation at position 331 selected from P331A, P331D, P331E, P331H, P331Q and P331S.

In some embodiments, the mutation at position 234 is L234F. In some embodiments, the mutation at position 268 is H268Q. In some embodiments, the mutation at position 327 is A327G. In some embodiments, the mutation at position 330 is A330K or A330T. In some embodiments, the mutation at position 331 is P331S.

In certain embodiments, the heterodimeric Fc variant is a Strategy 2 variant as described in any one of the embodiments above in which the first Fc polypeptide further comprises a mutation at one or more of positions 235, 237, 239, 264, 266, 267, 269, 270, 271, 272, 273, 323, 326 and/or 332. In some embodiments, the mutation at position 235 selected from L235A, L235D, L235E, L235F, L235H, L235I, L235P, L235Q, L235S, L235T, L235V, L235W and L235Y; the mutation at position 237 selected from G237A, G237F, G237L, G237N, G237T, G237W and G237Y; the mutation at position 239 selected from S239A, S239D, S239E, S239G, S239I, S239L, S239N, S239Q, S239R and S239V; the mutation at position 264 selected from V264A, V264F, V264I, V264L and V264T; the mutation at position 266 is V266I; the mutation at position 267 selected from S267A, S267G, S267H, S267I, S267N, S267P, S267T and S267V; the mutation at position 269 selected from E269A, E269D, E269F, E269G, E269H, E269I, E269K, E269L, E269N, E269P, E269Q, E269R, E269S, E269T, E269V, E269W and E269Y; the mutation at position 270 selected from D270A, D270E, D270F, D270H, D270I, D270N, D270Q, D270S, D270T, D270W and D270Y; the mutation at position 271 selected from P271D, P271E, P271G, P271H, P271I, P271K, P271L, P271N, P271Q, P271R, P271V and P271W; the mutation at position 272 selected from E272A, E272D, E272F, E272G, E272H, E272I, E272L, E272N, E272S, E272T, E272V, E272W and E272Y; the mutation at position 273 is V273A; the mutation at position 323 selected from V323A, V323I and V323L; the mutation at position 326 selected from K326A, K326D, K326H, K326N, K326Q, K326R, K326S and K326T, and the mutation at position 332 selected from I332A, I332L, I332T and I332V.

In certain embodiments, the heterodimeric Fc variant is a Strategy 2 variant as described in any one of the embodiments above in which the first Fc polypeptide further comprises a mutation at position 235 selected from L235A, L235D, L235E, L235F, L235H, L235I, L235P, L235Q, L235S, L235T, L235V, L235W and L235Y. In some embodiments, the mutation at position 235 is L235D.

In certain embodiments, the heterodimeric Fc variant is a Strategy 2 variant as described in any one of the embodiments above in which the first Fc polypeptide further comprises a mutation at position 237 selected from G237A, G237F, G237L, G237N, G237T, G237W and G237Y.

In certain embodiments, the heterodimeric Fc variant is a Strategy 2 variant as described in any one of the embodiments above in which the first Fc polypeptide further comprises a mutation at position 239 selected from S239A, S239D, S239E, S239G, S239I, S239L, S239N, S239Q, S239R and S239V.

In certain embodiments, the heterodimeric Fc variant is a Strategy 2 variant as described in any one of the embodiments above in which the first Fc polypeptide further comprises a mutation at position 264 selected from V264A, V264F, V264I, V264L and V264T.

In certain embodiments, the heterodimeric Fc variant is a Strategy 2 variant as described in any one of the embodiments above in which the first Fc polypeptide further comprises the mutation V266I.

In certain embodiments, the heterodimeric Fc variant is a Strategy 2 variant as described in any one of the embodiments above in which the first Fc polypeptide further comprises a mutation at position 267 selected from S267A, S267G, S267H, S267I, S267N, S267P, S267T and S267V. In some embodiments, the mutation at position 267 is S267A.

In certain embodiments, the heterodimeric Fc variant is a Strategy 2 variant as described in any one of the embodiments above in which the first Fc polypeptide further comprises a mutation at position 269 selected from E269A, E269D, E269F, E269G, E269H, E269I, E269K, E269L, E269N, E269P, E269Q, E269R, E269S, E269T, E269V, E269W and E269Y.

In certain embodiments, the heterodimeric Fc variant is a Strategy 2 variant as described in any one of the embodiments above in which the first Fc polypeptide further comprises a mutation at position 270 selected from D270A, D270E, D270F, D270H, D270I, D270N, D270Q, D270S, D270T, D270W and D270Y.

In certain embodiments, the heterodimeric Fc variant is a Strategy 2 variant as described in any one of the embodiments above in which the first Fc polypeptide further comprises a mutation at position 271 selected from P271D, P271E, P271G, P271H, P271I, P271K, P271L, P271N, P271Q, P271R, P271V and P271W.

In certain embodiments, the heterodimeric Fc variant is a Strategy 2 variant as described in any one of the embodiments above in which the first Fc polypeptide further comprises a mutation at position 272 selected from E272A, E272D, E272F, E272G, E272H, E272I, E272L, E272N, E272S, E272T, E272V, E272W and E272Y.

In certain embodiments, the heterodimeric Fc variant is a Strategy 2 variant as described in any one of the embodiments above in which the first Fc polypeptide further comprises the mutation V273A.

In certain embodiments, the heterodimeric Fc variant is a Strategy 2 variant as described in any one of the embodiments above in which the first Fc polypeptide further comprises a mutation at position 323 selected from V323A, V323I and V323L.

In certain embodiments, the heterodimeric Fc variant is a Strategy 2 variant as described in any one of the embodiments above in which the first Fc polypeptide further comprises a mutation at position 326 selected from K326A, K326D, K326H, K326N, K326Q, K326R, K326S and K326T.

In certain embodiments, the heterodimeric Fc variant is a Strategy 2 variant as described in any one of the embodiments above in which the first Fc polypeptide further comprises a mutation at position 332 selected from I332A, I332L, I332T and I332V.

In certain embodiments, the heterodimeric Fc variant is a Strategy 2 variant as described in any one of the embodiments above in which the second Fc polypeptide further comprises a mutation at one or more of positions 234, 235, 237, 240, 264, 269, 271, 272 and/or 273. In some embodiments, the mutation at position 234 selected from L234A, L234D, L234E, L234F, L234G, L234I, L234N, L234P, L234Q, L234S, L234T, L234V, L234W and L234Y; the mutation at position 235 selected from L235A, L235D, L235F, L235G, L235H, L235N, L235W and L235Y; the mutation at position 237 selected from G237A, G237D, G237E, G237F, G237H, G237I, G237K, G237L, G237N, G237Q, G237R, G237S, G237T, G237V, G237W and G237Y; the mutation at position 240 selected from V240I, V240L and V240T; the mutation at position 264 selected from V264L and V264T; the mutation at position 269 selected from E269D, E269T and E269V; the mutation at position 271 is P271G; the mutation at position 272 selected from E272A, E272D, E272I, E272K, E272L, E272P, E272Q, E272R, E272T and E272V, and the mutation at position 273 selected from V273A, V273I, V273L and V273T.

In certain embodiments, the heterodimeric Fc variant is a Strategy 2 variant as described in any one of the embodiments above in which the second Fc polypeptide further comprises a mutation at position 234 selected from L234A, L234D, L234E, L234F, L234G, L234I, L234N, L234P, L234Q, L234S, L234T, L234V, L234W and L234Y.

In certain embodiments, the heterodimeric Fc variant is a Strategy 2 variant as described in any one of the embodiments above in which the second Fc polypeptide further comprises a mutation at position 235 selected from L235A, L235D, L235F, L235G, L235H, L235N, L235W and L235Y.

In certain embodiments, the heterodimeric Fc variant is a Strategy 2 variant as described in any one of the embodiments above in which the second Fc polypeptide further comprises a mutation at position 237 selected from G237A, G237D, G237E, G237F, G237H, G237I, G237K, G237L, G237N, G237Q, G237R, G237S, G237T, G237V, G237W and G237Y. In some embodiments, the mutation at position 237 is G237D or G237L.

In certain embodiments, the heterodimeric Fc variant is a Strategy 2 variant as described in any one of the embodiments above in which the second Fc polypeptide further comprises a mutation at position 240 selected from V240I, V240L and V240T.

In certain embodiments, the heterodimeric Fc variant is a Strategy 2 variant as described in any one of the embodiments above in which the second Fc polypeptide further comprises a mutation at position 264 selected from V264L and V264T.

In certain embodiments, the heterodimeric Fc variant is a Strategy 2 variant as described in any one of the embodiments above in which the second Fc polypeptide further comprises a mutation at position 269 selected from E269D, E269T and E269V.

In certain embodiments, the heterodimeric Fc variant is a Strategy 2 variant as described in any one of the embodiments above in which the second Fc polypeptide further comprises the mutation P271G.

In certain embodiments, the heterodimeric Fc variant is a Strategy 2 variant as described in any one of the embodiments above in which the second Fc polypeptide further comprises a mutation at position 272 selected from E272A, E272D, E272I, E272K, E272L, E272P, E272Q, E272R, E272T and E272V.

In certain embodiments, the heterodimeric Fc variant is a Strategy 2 variant as described in any one of the embodiments above in which the second Fc polypeptide further comprises a mutation at position 273 selected from V273A, V273I, V273L and V273T.

In certain embodiments, the heterodimeric Fc variant is a Strategy 2 variant as described in any one of the embodiments described above and further comprises replacement of the native loop at positions 325 to 331 in the second Fc polypeptide with a polypeptide loop of between 7 and 15 amino acids in length or between 8 and 15 amino acids in length as described in any one of the embodiments provided above under “Asymmetric Loop Replacement.”

In certain embodiments, the polypeptide loop comprised by the second Fc polypeptide of the Strategy 2 variant comprises an amino acid sequence as set forth in any one of Formula (I), Formula (Ia), Formula (Ib), Formula (II), Formula (ITT), Formula (IV), Formula (V) or Formula (VI), as described above under “Asymmetric Loop Replacement.” In some embodiments, the polypeptide loop comprised by the second Fc polypeptide comprises an amino acid sequence as set forth in any one of the sequences shown in Tables 3A and 3B (SEQ ID NOs: 4-172). In some embodiments, the polypeptide loop comprised by the second Fc polypeptide comprises an amino acid sequence as set forth in any one of SEQ ID NOs: 4-90 (see Table 3A above). In some embodiments, the polypeptide loop comprised by the second Fc polypeptide comprises an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 (see Table 3A above).

In certain embodiments, the heterodimeric Fc variant is a Strategy 2 variant and comprises the amino acid mutations as set out in Table 5A, Table 5B and Table 5C for any one of the variants listed under “Strategy 2” and “Strategy 1/3+Strategy 2 Combinations.” In certain embodiments, the heterodimeric Fc variant is a Strategy 2 variant and comprises the amino acid mutations as set out for any one of the variants shown in Table 6.23 or Table 6.26. In some embodiments, the heterodimeric Fc variant is a Strategy 2 variant and comprises the amino acid mutations as set out for any one of the variants shown in Table 6.23.

In certain embodiments, the heterodimeric Fc variant is a Strategy 2 variant and comprises the amino acid mutations of any one of the variants shown in Table 6.18 that has a “IIb Selectivity Fold wrt Control” value≥0.5 and a “IIb-Fold wrt Control” value≥0.5 (“Criteria B”). In some embodiments, the heterodimeric Fc variant comprises the amino acid mutations of any one of the variants shown in Table 6.18 that has a “IIb Selectivity Fold wrt Control” value≥1.0 and a “IIb-Fold wrt Control” value≥0.3 (“Criteria C”). In certain embodiments, the heterodimeric Fc variant comprises the amino acid mutations of any one of the variants shown in Table 6.18 that has a “IIb Selectivity Fold wrt Control” value≥1.0 and a “IIb-Fold wrt Control” value≥0.5 (“Criteria D”). In certain embodiments, the heterodimeric Fc variant comprises the amino acid mutations of any one of the variants shown in Table 6.18 that has a “IIb Selectivity Fold wrt Control” value≥1.5 and a “IIb-Fold wrt Control” value≥0.3 (“Criteria A”).

### Combination Variants

As described in the Examples provided herein, mutations comprised by Strategy 1/3 variants can be combined with mutations comprised by Strategy 2 variants to provide heterodimeric Fc variants having increased selectivity, and optionally increased affinity, for FcγRIIb. In certain embodiments, the heterodimeric Fc variant is a combination variant and comprises mutations from a Strategy 1/3 variant in one Fc polypeptide and mutations from a Strategy 2 variant in the other Fc polypeptide.

In certain embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331, in which
(i) the mutation at position 234 is selected from L234A, L234F, L234G, L234H, L234I, L234N, L234P, L234Q, L234S, L234T, L234V, L234W and L234Y,
(ii) the mutation at position 268 is selected from H268A, H268D, H268E, H268F, H268G, H268I, H268K, H268L, H268N, H268P, H268Q, H268R, H268S, H268T, H268V, H268W and H268Y,
(iii) the mutation at position 327 is selected from A327G and A327E;
(iv) the mutation at position 330 is selected from A330K, A330H, A330Q, A330R, A330S and A330T, and
(v) the mutation at position 331 is selected from P331A, P331D, P331E, P331H, P331Q and P331S, and
(b) a second Fc polypeptide comprising mutations from a Strategy 1/3 variant, the mutations comprising the mutation G236D, and replacement of the native loop at positions 325 to 331 with a polypeptide loop of between 7 and 15 amino acids in length or between 8 and 15 amino acids in length as described in any one of the embodiments provided above under “Asymmetric Loop Replacement.”

In some embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331 as described above, and
(b) a second Fc polypeptide comprising mutations from a Strategy 1/3 variant, the mutations comprising the mutation G236D, replacement of the native loop at positions 325 to 331 with a polypeptide loop of between 7 and 15 amino acids in length or between 8 and 15 amino acids in length as described in any one of the embodiments provided above under “Asymmetric Loop Replacement” and one or more binding enhancers as described above.

In some embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331 as described above, and
(b) a second Fc polypeptide comprising mutations from a Strategy 1/3 variant, the mutations comprising the mutation G236D, replacement of the native loop at positions 325 to 331 with a polypeptide loop of between 7 and 15 amino acids in length or between 8 and 15 amino acids in length as described in any one of the embodiments provided above under “Asymmetric Loop Replacement” and one or more binding enhancers selected from S239D, S239E, V266I, S267I, S267Q, S267V and H268D.

In some embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331 as described above, and
(b) a second Fc polypeptide comprising mutations from a Strategy 1/3 variant, the mutations comprising the mutation G236D, replacement of the native loop at positions 325 to 331 with a polypeptide loop of between 7 and 15 amino acids in length or between 8 and 15 amino acids in length as described in any one of the embodiments provided above under “Asymmetric Loop Replacement” and (i) the mutation S239D or S239E, and/or (ii) the mutation H268D, and/or (iii) the mutation S267I or S267V.

In some embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331 as described above, and
(b) a second Fc polypeptide comprising mutations from a Strategy 1/3 variant, the mutations comprising the mutation G236D, replacement of the native loop at positions 325 to 331 with a polypeptide loop of between 7 and 15 amino acids in length or between 8 and 15 amino acids in length as described in any one of the embodiments provided above under “Asymmetric Loop Replacement” and the mutations S239D and H268D.

In some embodiments, the heterodimeric Fc variant is a combination variant and comprises:
(a) a first Fc polypeptide comprising mutations from a Strategy 2 variant, the mutations comprising the mutation G236N, and a mutation at one or more positions selected from 234, 268, 327, 330 and 331 as described above, and
(b) a second Fc polypeptide comprising mutations from a Strategy 1/3 variant, the mutations comprising the mutation G236D, replacement of the native loop at positions 325 to 331 with a polypeptide loop of between 7 and 15 amino acids in length or between 8 and 15 amino acids in length as described in any one of the embodiments provided above under “Asymmetric Loop Replacement” and the mutations S239D, H268D and S267V.

In certain embodiments, in the combination variant, the mutation at position 234 in the first Fc polypeptide is L234F. In some embodiments, in the combination variant, the mutation at position 268 in the first Fc polypeptide is H268Q. In some embodiments, in the combination variant, the mutation at position 327 in the first Fc polypeptide is A327G. In some embodiments, in the combination variant, the mutation at position 330 in the first Fc polypeptide is A330K or A330T. In some embodiments, in the combination variant, the mutation at position 331 in the first Fc polypeptide is P331S.

In certain embodiments, in the combination variant, the polypeptide loop comprised by the second Fc polypeptide comprises an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations. In some embodiments, in the combination variant, the polypeptide loop comprised by the second Fc polypeptide comprises an amino acid sequence as set forth in any one of SEQ ID NOs: 4, 5, 6, 7, 8, 9, 10, 11, 12, 13 or 14. In some embodiments, the polypeptide loop comprised by the second Fc polypeptide comprises an amino acid sequence as set forth in any one of Formula (I), Formula (Ia), Formula (Ib), Formula (II), Formula (III), Formula (IV), Formula (V) or Formula (VI), as described above under “Asymmetric Loop Replacement.” In some embodiments, the polypeptide loop comprised by the second Fc polypeptide comprises an amino acid sequence as set forth in any one of the sequences shown in Tables 3A and 3B (SEQ ID NOs: 4-172). In some embodiments, the polypeptide loop comprised by the second Fc polypeptide comprises an amino acid sequence as set forth in any one of SEQ ID NOs: 4-90 (see Table 3A above). In some embodiments, the polypeptide loop comprised by the second Fc polypeptide comprises an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 (see Table 3A above).

In certain embodiments, the heterodimeric Fc variant is a combination variant and comprises the amino acid mutations as set out in Table 5A or Table 5C for any one of the variants listed under “Strategy 1/3+Strategy 2 Combinations.”

TABLE 5A
Exemplary Variants having Increased Selectivity for FcγRIIb

| Variant | Mutations¹ | |
|---|---|---|
| # | Chain A | Chain B |

LOOP REPLACEMENT + SYMMETRICAL 236 MUTATION

| | | |
|---|---|---|
| 22107 | L234D_G236N | Template 1 + G236N_S239D_S267I_H268D |
| 22108 | L234D_G236D | Template 1 + G236D_S239D_S267I_H268D |
| 22112 | L234D_G236D_G237F | Template 1 + G236D_G237F_S239D_S267I_H268D |
| 22113 | L234D_G236D_G237W | Template 1 + G236D_G237W_S239D_S267I_H268D |

ASYMMETRIC 236 MUTATION

| | | |
|---|---|---|
| 19509 | G236N | S239D_H268D |
| 19512 | | G236N_S239D_H268D |
| 19517 | G236D | S239D_H268D |
| 19518 | | G236D_S239D_H268D |
| 19694 | | G236D_G237F_S239D_H268D |
| 22074 | L234D_S267A | G236D_S239D_V266L_S267A_H268D |
| 19521 | G236N | G236D_S239D_H268D |
| 19523 | G236D | G236N_S239D_H268D |
| 19525 | G236D | G236Q_S239D_H268D |
| 19526 | G236D | G236K_S239D_H268D |
| 19527 | G236N | G236K_S239D_H268D |
| 19528 | G236N | G236S_S239D_H268D |
| 19530 | G236D | G236E_S239D_H268D |
| 19531 | G236D | G236H_S239D_H268D |
| 22076 | L234D_G236N_S267A | S239D_V266L_S267A_H268D |
| 19540 | L234D_G236N_S267A | G236D_S239D_V266L_S267A_H268D |
| 22073 | G236N_S267A | G236D_S239D_V266L_S267A_H268D |
| 19535 | L234D_G236N | G236D_S239D_S267I_H268D |
| 22075 | L234D_G236N | G236D_S239D_V266L_S267A_H268D |
| 22077 | L234D_G236N_S267A | G236D_S239D_S267A_H268D |
| 22078 | L234D_G236N_S267A | G236D_S239D_V266L_H268D |
| 22079 | L234D_G236N_S267A | G236D_S239D_V266L_S267A |
| 22131 | L234F_G236N_S267A | G236D_S239D_V266L_S267A_H268D |
| 22116 | L234D_G236N_S267A_{—} | G236D_S239D_V266L_S267A_H268D_K326D |
| | K326D | |
| 27362 | G236N_G237A | G236D_G237F_S239D_S267V_H268D |

STRATEGY ⅓

| | | |
|---|---|---|
| 19522 | G236N | Template 1 + G236D_S239D_H268D |
| 22068 | G236N | Template 1 + G236D_S239D_S267I_H268D |
| 22095 | L234D_G236N | Template 7 + G236D_S239D_S267I_H268D |
| 22096 | L234D_G236N | Template 66 + G236D_S239D_S267I_H268D |
| 22097 | L234D_G236N | Template 151 + G236D_S239D_S267I_H268D |
| 22069 | L234D | Template 1 + G236D_S239D_S267I_H268D |
| 22070 | L234D_G236N | Template 1 + S239D_S267I_H268D |
| 19544 | L234D_G236N | Template 1 + G236D_S239D_S267I_H268D |
| 22071 | L234D_G236N | Template 1 + G236D_S239D_H268D |
| 22072 | L234D_G236N | Template 1 + G236D_S239D_S267I |
| 22098 | L234D_G236N | Template 1 + G236D_S239D_S267A_H268D |
| 22100 | L234D_G236N | Template 1 + G236D_S239D_V266L_S267I_H268D |
| 22101 | L234D_G236N | Template 1 + G236D_S239D_V266L_S267A_H268D |
| 22109 | L234D_G236N | Template 1 + G236D_S239D_S267V_H268D |
| 22110 | L234D_G236N | Template 1 + G236D_G237F_S239D_S267I_H268D |
| 22099 | L234D_G236N_S267A | Template 1 + G236D_S239D_S267I_H268D |
| 22104 | L234D_G236N_A330K | Template 1 + G236D_S239D_S267I_H268D |
| 22105 | L234D_G236N_K326D | Template 1 + G236D_S239D_S267I_H268D |
| 22121 | L234D_G236N_G237A | Template 1 + G236D_S239D_S267I_H268D |
| 22122 | L234D_G236N_G237A | Template 1 + G236D_G237F_S239D_S267I_H268D |
| 22106 | L234D_G236N_K326D_{—} | Template 1 + G236D_S239D_S267I_H268D |
| | A330K | |
| 27293 | G236N_G237A | Template 1 + G236D_G237F_S239D_S267V_H268D |
| 26505 | G236N_G237A | Template 1 (G330*P) + G236D_G237F_S239D_S267V_H268D |
| 26489 | G236N_G237A | Template 1 (G330*V) + G236D_G237F_S239D_S267V_H268D |
| 26488 | G236N_G237A | Template 1 (G330*A) + G236D_G237F_S239D_S267V_H268D |
| 26490 | G236N_G237A | Template 1 (G330*L) + G236D_G237F_S239D_S267V_H268D |
| 26491 | G236N_G237A | Template 1 (G330*I) + G236D_G237F_S239D_S267V_H268D |
| 26492 | G236N_G237A | Template 1 (G330*M) + G236D_G237F_S239D_S267V_H268D |
| 26493 | G236N_G237A | Template 1 (G330*F) + G236D_G237F_S239D_S267V_H268D |
| 26494 | G236N_G237A | Template 1 (G330*W) + G236D_G237F_S239D_S267V_H268D |
| 26495 | G236N_G237A | Template 1 (G330*Y) + G236D_G237F_S239D_S267V_H268D |
| 26496 | G236N_G237A | Template 1 (G330*T) + G236D_G237F_S239D_S267V_H268D |
| 26497 | G236N_G237A | Template 1 (G330*S) + G236D_G237F_S239D_S267V_H268D |
| 26498 | G236N_G237A | Template 1 (G330*Q) + G236D_G237F_S239D_S267V_H268D |
| 26499 | G236N_G237A | Template 1 (G330*N) + G236D_G237F_S239D_S267V_H268D |
| 26500 | G236N_G237A | Template 1 (G330*D) + G236D_G237F_S239D_S267V_H268D |
| 26501 | G236N_G237A | Template 1 (G330*E) + G236D_G237F_S239D_S267V_H268D |
| 26502 | G236N_G237A | Template 1 (G330*R) + G236D_G237F_S239D_S267V_H268D |
| 26503 | G236N_G237A | Template 1 (G330*K) + G236D_G237F_S239D_S267V_H268D |
| 26504 | G236N_G237A | Template 1 (G330*H) + G236D_G237F_S239D_S267V_H268D |
| 26470 | G236N_G237A | Template 1 (D329*G) + G236D_G237F_S239D_S267V_H268D |
| 26471 | G236N_G237A | Template 1 (D329*A) + G236D_G237F_S239D_S267V_H268D |
| 26487 | G236N_G237A | Template 1 (D329*P) + G236D_G237F_S239D_S267V_H268D |
| 26472 | G236N_G237A | Template 1 (D329*V) + G236D_G237F_S239D_S267V_H268D |
| 26473 | G236N_G237A | Template 1 (D329*L) + G236D_G237F_S239D_S267V_H268D |
| 26474 | G236N_G237A | Template 1 (D329*I) + G236D_G237F_S239D_S267V_H268D |
| 26475 | G236N_G237A | Template 1 (D329*M) + G236D_G237F_S239D_S267V_H268D |
| 26476 | G236N_G237A | Template 1 (D329*F) + G236D_G237F_S239D_S267V_H268D |
| 26477 | G236N_G237A | Template 1 (D329*W) + G236D_G237F_S239D_S267V_H268D |
| 26478 | G236N_G237A | Template 1 (D329*Y) + G236D_G237F_S239D_S267V_H268D |
| 26479 | G236N_G237A | Template 1 (D329*T) + G236D_G237F_S239D_S267V_H268D |
| 26480 | G236N_G237A | Template 1 (D329*S) + G236D_G237F_S239D_S267V_H268D |
| 26481 | G236N_G237A | Template 1 (D329*Q) + G236D_G237F_S239D_S267V_H268D |
| 26482 | G236N_G237A | Template 1 (D329*N) + G236D_G237F_S239D_S267V_H268D |
| 26483 | G236N_G237A | Template 1 (D329*E) + G236D_G237F_S239D_S267V_H268D |
| 26484 | G236N_G237A | Template 1 (D329*R) + G236D_G237F_S239D_S267V_H268D |
| 26485 | G236N_G237A | Template 1 (D329*K) + G236D_G237F_S239D_S267V_H268D |
| 26486 | G236N_G237A | Template 1 (D329*H) + G236D_G237F_S239D_S267V_H268D |
| 29688, | G236N_G237D | Template 1 (D329*I) + G236D_G237F_S239D_S267V_H268D |
| 31186 | | |
| 29689, | L235F_G236N_G237A | Template 1 (D329*I) + G236D_G237F_S239D_S267V_H268D |
| 31187 | | |
| 29690 | G236N_G237A_S239Y | Template 1 (D329*I) + G236D_G237F_S239D_S267V_H268D |
| 29691 | L234D_G236N_G237A | Template 1 (D329*I) + G236D_G237F_S239D_S267V_H268D |
| 29692 | G236N_G237A_S239G | Template 1 (D329*I) + G236D_G237F_S239D_S267V_H268D |
| 29693 | G236N_G237L | Template 1 (D329*I) + G236D_G237F_S239D_S267V_H268D |
| 29694 | G236N_G237D | Template 1 (G330*K) + G236D_G237F_S239D_S267V_H268D |
| 29695, | L235F_G236N_G237A | Template 1 (G330*K) + G236D_G237F_S239D_S267V_H268D |
| 31188 | | |
| 29696 | G236N_G237A_S239Y | Template 1 (G330*K) + G236D_G237F_S239D_S267V_H268D |
| 29697 | L234D_G236N_G237A | Template 1 (G330*K) + G236D_G237F_S239D_S267V_H268D |
| 29698 | G236N_G237A_S239G | Template 1 (G330*K) + G236D_G237F_S239D_S267V_H268D |
| 29699 | G236N_G237L | Template 1 (G330*K) + G236D_G237F_S239D_S267V_H268D |
| 29700 | G236N_G237D | Template 1 + G236D_G237F_S239D_S267V_H268D_I332L |
| 29701 | L235F_G236N_G237A | Template 1 + G236D_G237F_S239D_S267V_H268D_I332L |
| 29702 | G236N_G237A_S239Y | Template 1 + G236D_G237F_S239D_S267V_H268D_I332L |
| 29703 | L234D_G236N_G237A | Template 1 + G236D_G237F_S239D_S267V_H268D_I332L |
| 29704 | G236N_G237A_S239G | Template 1 + G236D_G237F_S239D_S267V_H268D_I332L |
| 29705 | G236N_G237L | Template 1 + G236D_G237F_S239D_S267V_H268D_I332L |
| 29706 | G236N_G237A_E269K | Template 1 (D329*I) + G236D_G237F_S239D_S267V_H268D |
| 29707 | G236N_G237A_E269K | Template 1 (G330*K) + G236D_G237F_S239D_S267V_H268D |
| 29708 | G236N_G237A_E269K | Template 1 + G236D_G237F_S239D_S267V_H268D_I332L |
| 29709 | G236N_G237A_S239H | Template 1 (D329*I) + G236D_G237F_S239D_S267V_H268D |
| 29710 | G236N_G237A_S239H | Template 1 (G330*K) + G236D_G237F_S239D_S267V_H268D |
| 29711 | G236N_G237A_S239H | Template 1 + G236D_G237F_S239D_S267V_H268D_I332L |
| 31210 | L235F_G236N_G237A | Template 1 (D329*I) + G236D_G237F_S239D_H268D |
| 31209 | L235F_G236N_G237A | Template 1 (F328*Y_D329*I) + G236D_G237F_S239D_ S267V_H268D |
| 31211 | L235F_G236N_G237D | Template 1 (D329*I) + G236D_G237F_S239D_S267V_H268D |
| 31212 | L235F_G236N_G237A | Template 1 (D329*I) + G236D_G237F_S239D_S267V_H268D_P271D |
| 31213 | L235F_G236N_G237A | Template 1 (D329*I) + G236D_G237F_S239D_S267V_H268D_I332L |
| 31214 | L235F_G236N_G237A | Template 1 (D329*I_G330*K) + G236D_G237F_S239D_S267V_H268D |
| 31216 | L235F_G236F | Template 1 (D329*I) + G236D_G237F_S239D_S267V_H268D |
| 31274 | L235F_G236N_G237A_{—} | Template 1 (D329*I) + G236D_G237F_S239D_T250V_S267V_H268D_A287F |
| | T250V_A287F | |
| 31275 | L235F_G236N_G237A_{—} | Template 1 (D329*I) + G236D_G237F_S239D_T250V_S267V_H268D_M428F |
| | T250V_M428F | |
| 31276 | L235F_G236N_G237A_{—} | Template 1 (D329*I) + G236D_G237F_S239D_S267V_H268D_A287F_M428F |
| | A287F_M428F | |
| 32210 | G236N_G237D | Template 1 (D329*I) + G236D_G237F_S239D_S267V_H268D_I332L |
| 32211 | G236N_G237E | Template 1 (D329*I) + G236D_G237F_S239D_S267V_H268D_I332L |
| 32212 | G236N_G237G | Template 1 (D329*I) + G236D_G237F_S239D_S267V_H268D_I332L |
| 32226 | L235D_G236N_G237A | Template 1 (D329*I) + G236D_G237F_S239D_S267V_H268D_I332L |
| 32227 | L235E_G236N_G237A | Template 1 (D329*I) + G236D_G237F_S239D_S267V_H268D_I332L |
| 32230 | L235V_G236N_G237A | Template 1 (D329*I) + G236D_G237F_S239D_S267V_H268D_I332L |
| 32231 | L235Y_G236N_G237A | Template 1 (D329*I) + G236D_G237F_S239D_S267V_H268D_I332L |
| 32242 | G236N_G237A_S239P | Template 1 (D329*I) + G236D_G237F_S239D_S267V_H268D_I332L |
| 29724, | G236N_G237D | Template 7 (E328*H_E329*R_A331*BY) + G236D_G237F_S239D_S267V_H268D |
| 29725 | L235F_G236N_G237A | Template 7 (E328*H_E329*R_A331*BY) + G236D_G237F_S239D_S267V_H268D |
| 29726 | G236N_G237A_S239Y | Template 7 (E328*H_E329*R_A331*BY) + G236D_G237F_S239D_S267V_H268D |
| 31255 | G236N_G237D_A287F_{—} | Template 7 (E328*H_E329*R_A331*BY) + G236D_G237F_S239D_S267V_H268D_{—} |
| | M428F | A287F_M428F |
| 32282 | L234D_G236N_G237A | Template 7 (E328*H_E329*R_A331*BY) + G236D_G237F_S239D_S267V_H268D |
| 32284 | L235D_G236N_G237A | Template 7 (E328*H_E329*R_A331*BY) + G236D_G237F_S239D_S267V_H268D |
| 32287 | G236N_G237A_S239G | Template 7 (E328*H_E329*R_A331*BY) + G236D_G237F_S239D_S267V_H268D |
| 32288 | G236N_G237A_S239H | Template 7 (E328*H_E329*R_A331*BY) + G236D_G237F_S239D_S267V_H268D |
| 32296 | G236N_G237E | Template 7 (E328*H_E329*R_A331*BY) + G236D_G237F_S239D_S267V_H268D |

STRATEGY 2

| | | |
|---|---|---|
| 19585 | L234F_G236N_H268Q_{—} | L234F_G236D_S239D_V266L_S267A_H268D_K274Q_A327G_A330S_P331S |
| | K274Q_A327G_A330K_{—} | |
| | P331S | |
| 22130 | L234F_G236N_H268Q_{—} | L234F_S239D_V266L_S267A_H268D_K274Q_A327G_A330S_P331S |
| | K274Q_A327G_A330K_{—} | |
| | P331S | |
| 22081 | L234F_H268Q_K274Q_{—} | L234F_G236D_S239D_V266L_S267A_H268D_K274Q_A327G_A330S_P331S |
| | A327G_A330K_P331S | |
| 31278 | L234F_H268Q_A327G_{—} | G236D_G237D_S239D_V266L_S267A_H268D |
| | P329I_A330K_P331S | |
| 22084 | L234F_G236N_H268Q_{—} | L234F_G236D_S239D_V266L_S267A_H268D_K274Q_A327G_A330S_P331S |
| | K274Q_A327G_P331S | |
| 22094 | L234F_G236N_H268Q_{—} | L234F_G236D_S239D_V266L_S267A_H268D_K274Q_A327G_A330S |
| | K274Q_A327G_A330K_{—} | |
| | P331S | |
| 22080 | G236N_H268Q_K274Q_{—} | L234F_G236D_S239D_V266L_S267A_H268D_K274Q_A327G_A330S_P331S |
| | A327G_A330K_P331S | |
| 22082 | L234F_G236N_H268Q_{—} | L234F_G236D_S239D_V266L_S267A_H268D_K274Q_A327G_A330S_P331S |
| | A327G_A330K_P331S | |
| 22083 | L234F_G236N_H268Q_{—} | L234F_G236D_S239D_V266L_S267A_H268D_K274Q_A327G_A330S_P331S |
| | K274Q_A330K_P331S | |
| 22085 | L234F_G236N_H268Q_{—} | L234F_G236D_S239D_V266L_S267A_H268D_K274Q_A327G_A330S_P331S |
| | K274Q_A327G_A330K | |
| 22086 | L234F_G236N_H268Q_{—} | G236D_S239D_V266L_S267A_H268D_K274Q_A327G_A330S_P331S |
| | K274Q_A327G_A330K_{—} | |
| | P331S | |
| 22088 | L234F_G236N_H268Q_{—} | L234F_G236D_S239D_S267A_H268D_K274Q_A327G_A330S_P331S |
| | K274Q_A327G_A330K_{—} | |
| | P331S | |
| 22089 | L234F_G236N_H268Q_{—} | L234F_G236D_S239D_V266L_H268D_K274Q_A327G_A330S_P331S |
| | K274Q_A327G_A330K_{—} | |
| | P331S | |
| 22090 | L234F_G236N_H268Q_{—} | L234F_G236D_S239D_V266L_S267A_K274Q_A327G_A330S_P331S |
| | K274Q_A327G_A330K_{—} | |
| | P331S | |
| 22091 | L234F_G236N_H268Q_{—} | L234F_G236D_S239D_V266L_S267A_H268D_A327G_A330S_P331S |
| | K274Q_A327G_A330K_{—} | |
| | P331S | |
| 22092 | L234F_G236N_H268Q_{—} | L234F_G236D_S239D_V266L_S267A_H268D_K274Q_A330S_P331S |
| | K274Q_A327G_A330K_{—} | |
| | P331S | |
| 22093 | L234F_G236N_H268Q_{—} | L234F_G236D_S239D_V266L_S267A_H268D_K274Q_A327G_P331S |
| | K274Q_A327G_A330K_{—} | |
| | P331S | |
| 22117 | L234D_G236N_H268Q_{—} | L234F_G236D_S239D_V266L_S267A_H268D_K274Q_A327G_A330S_P331S |
| | K274Q_A327G_A330K_{—} | |
| | P331S | |
| 22119 | L234F_G236N_H268Q_{—} | L234F_G236D_S239D_V266L_S267I_H268D_K274Q_A327G_A330S_P331S |
| | K274Q_A327G_A330K_{—} | |
| | P331S | |
| 22120 | L234F_G236N_H268Q_{—} | L234F_G236D_S239D_V266L_S267A_H268D_K274Q_A327G_A330S_P331S_K326D |
| | K274Q_A327G_A330K_{—} | |
| | P331S_K326D | |
| 22102 | L234D_G236N_H268Q_{—} | Template 1 + L234F_G236D_S239D_V266L_S267I_H268D_K274Q |
| | K274Q_A327G_A330K_{—} | |
| | P331S | |
| 22103 | L234F_G236N_H268Q_{—} | Template 1 + L234F_G236D_S239D_V266L_S267I_H268D_K274Q |
| | K274Q_A327G_A330K_{—} | |
| | P331S | |
| 22118 | L234F_G236N_H268Q_{—} | Template 1 + L234F_G236D_S239D_V266L_S267A_H268D_K274Q |
| | K274Q_A327G_A330K_{—} | |
| | P331S | |
| 22115 | L234D_G236N_S267A_{—} | G236D_S239D_V266L_S267I_H268D_L234F_K274Q_A327G_A330K_P331S |
| | H268Q_K274Q_A327G_{—} | |
| | A330K_P331S | |
| 27294 | L234F_G236N_H268Q_{—} | G236D_S239D_V266L_S267A_H268D |
| | A327G_A330K_P331S | |
| 29712 | L234F_L235D_G236N_{—} | G236D_G237D_S239D_V266L_S267A_H268D |
| | H268Q_A327G_A330K_{—} | |
| | P331S | |
| 29713 | L234F_G236N_S267A_{—} | G236D_G237D_S239D_V266L_S267A_H268D |
| | H268Q_A327G_A330K_{—} | |
| | P331S | |
| 29714 | L234F_G236N_H268Q_{—} | G236D_G237D_S239D_V266L_S267A_H268D |
| | A327G_A330T_P331S | |
| 29715, | L234F_G236N_H268Q_{—} | G236D_G237D_S239D_V266L_S267A_H268D |
| 31256 | A327G_P329I_A330K_{—} | |
| | P331S | |
| 29716, | L234F_L235D_G236N_{—} | G236D_G237L_S239D_V266L_S267A_H268D |
| 31190 | H268Q_A327G_A330K_{—} | |
| | P331S | |
| 29717 | L234F_G236N_S267A_{—} | G236D_G237L_S239D_V266L_S267A_H268D |
| | H268Q_A327G_A330K_{—} | |
| | P331S | |
| 29718 | L234F_G236N_H268Q_{—} | G236D_G237L_S239D_V266L_S267A_H268D |
| | A327G_A330T_P331S | |
| 29719 | L234F_G236N_H268Q_{—} | G236D_G237L_S239D_V266L_S267A_H268D |
| | A327G_P329I_A330K_{—} | |
| | P331S | |
| 31253 | L234F_G236N_H268Q_{—} | G236D_G237D_S239D_V266L_S267A_H268D_A287F_M428F |
| | A287F_A327G_P329I_{—} | |
| | A330K_P331S_M428F | |
| 32274 | L234F_G236N_H268Q_{—} | G236D_G237L_S239D_V266L_S267A_H268D |
| | A327G_P329A_A330K_{—} | |
| | P331S | |

STRATEGY ⅓ + STRATEGY 2 COMBINATIONS

| | | |
|---|---|---|
| 29727, | L234F_L235D_G236N_{—} | Template 1 (D329*I) + G236D_G237F_S239D_S267V_H268D |
| 31192 | H268Q_A327G_A330K_{—} | |
| | P331S | |
| 29728 | L234F_G236N_S267A_{—} | Template 1 (D329*I) + G236D_G237F_S239D_S267V_H268D |
| | H268Q_A327G_A330K_{—} | |
| | P331S | |
| 29729 | L234F_G236N_H268Q_{—} | Template 1 (D329*I) + G236D_G237F_S239D_S267V_H268D |
| | A327G_A330T_P331S | |
| 29730 | L234F_L235D_G236N_{—} | Template 1 (G330*K) + G236D_G237F_S239D_S267V_H268D |
| | H268Q_A327G_A330K_{—} | |
| | P331S | |
| 29731 | L234F_G236N_S267A_{—} | Template 1 (G330*K) + G236D_G237F_S239D_S267V_H268D |
| | H268Q_A327G_A330K_{—} | |
| | P331S | |
| 29732 | L234F_G236N_H268Q_{—} | Template 1 (G330*K) + G236D_G237F_S239D_S267V_H268D |
| | A327G_A330T_P331S | |
| 29733 | L234F_L235D_G236N_{—} | Template 1 + G236D_G237F_S239D_S267V_H268D_I332L |
| | H268Q_A327G_A330K_{—} | |
| | P331S | |
| 29734 | L234F_G236N_S267A_{—} | Template 1 + G236D_G237F_S239D_S267V_H268D I332L |
| | H268Q_A327G_A330K_{—} | |
| | P331S | |
| 29735 | L234F_G236N_H268Q_{—} | Template 1 + G236D_G237F_S239D_S267V_H268D_I332L |
| | A327G_A330T_P331S | |
| 32292 | L234F_L235D_G236N_{—} | Template 1 (D329*I) + G236D_G237F_S239D_S267V_H268D_I332L |
| | H268Q_A327G_A330K_{—} | |
| | P331S | |
| 32293 | L234F_G236N_S267A_{—} | Template 1 (D329*I) + G236D_G237F_S239D_S267V_H268D_I332L |
| | H268Q_A327G_A330K_{—} | |
| | P331S | |
| 32294 | L234F_G236N_H268Q_{—} | Template 1 (D329*I) + G236D_G237F_S239D_S267V_H268D_I332L |
| | A327G_A330T_P331S | |
| 32295 | L234F_G236N_H268Q_{—} | Template 1 (D329*I) + G236D_G237F_S239D_S267V_H268D_I332L |
| | A327G_P329I_A330K_{—} | |
| | P331S | |

¹The notation “Template” in the Mutations for Chain B indicates that residues 325-331 of the wild-type chain B sequence are replaced with the noted Template. When a Template comprises mutations, this is indicated in brackets after the Template number, for example, “Template 1 (D329*I)” indicates the polypeptide loop has the sequence of Template 1 in which D at position 329* is replaced by I.

TABLE 5B
Exemplary Variants having Increased Selectivity for FcγRIIb

| Variant | Mutations¹ | |
|---|---|---|
| # | Chain A | Chain B |

STRATEGY ⅓

| | | |
|---|---|---|
| 19544 | L234D_G236N | Template 1 + |
| | | G236D_S239D_S267I_H268D |
| 27293 | G236N_G237A | Template 1 + |
| | | G236D_G237F_S239D_S267V_H268D |
| 27362 | G236N_G237A | G236D_G237F_S239D_S267V_H268D |
| 26105 | L234D_G236N_G237A | Template 1 + |
| | | G236D_G237F_S239D_S267V_H268D |
| 26098 | L234F_G236N_G237A | Template 1 + |
| | | G236D_G237F_S239D_S267V_H268D |
| 26103 | L234Q_G236N_G237A | Template 1 + |
| | | G236D_G237F_S239D_S267V_H268D |
| 26101 | L234T_G236N_G237A | Template 1 + |
| | | G236D_G237F_S239D_S267V_H268D |
| 26099 | L234W_G236N_G237A | Template 1 + |
| | | G236D_G237F_S239D_S267V_H268D |
| 26112 | L235A_G236N_G237A | Template 1 + |
| | | G236D_G237F_S239D_S267V_H268D |
| 26123 | L235D_G236N_G237A | Template 1 + |
| | | G236D_G237F_S239D_S267V_H268D |
| 26124 | L235E_G236N_G237A | Template 1 + |
| | | G236D_G237F_S239D_S267V_H268D |
| 26116 | L235F_G236N_G237A | Template 1 + |
| | | G236D_G237F_S239D_S267V_H268D |
| 26127 | L235H_G236N_G237A | Template 1 + |
| | | G236D_G237F_S239D_S267V_H268D |
| 26125 | L235R_G236N_G237A | Template 1 + |
| | | G236D_G237F_S239D_S267V_H268D |
| 26117 | L235W_G236N_G237A | Template 1 + |
| | | G236D_G237F_S239D_S267V_H268D |
| 26118 | L235Y_G236N_G237A | Template 1 + |
| | | G236D_G237F_S239D_S267V_H268D |
| 26159 | G236N_G237D | Template 1 + |
| | | G236D_G237F_S239D_S267V_H268D |
| 26149 | G236N_G237L | Template 1 + |
| | | G236D_G237F_S239D_S267V_H268D |
| 26158 | G236N_G237N | Template 1 + |
| | | G236D_G237F_S239D_S267V_H268D |
| 26166 | G236N_G237A_S239A | Template 1 + |
| | | G236D_G237F_S239D_S267V_H268D |
| 26165 | G236N_G237A_S239G | Template 1 + |
| | | G236D_G237F_S239D_S267V_H268D |
| 26181 | G236N_G237A_S239H | Template 1 + |
| | | G236D_G237F_S239D_S267V_H268D |
| 26174 | G236N_G237A_S239T | Template 1 + |
| | | G236D_G237F_S239D_S267V_H268D |
| 26173 | G236N_G237A_S239Y | Template 1 + |
| | | G236D_G237F_S239D_S267V_H268D |
| 26235 | G236N_G237A | Template 1 + |
| | | G236K_G237F_S239D_S267V_H268D |
| 26381 | G236N_G237A | Template 1 + |
| | | G236D_G237F_S239D_S267V_H268D_P271D |
| 26392 | G236N_G237A | Template 1 + |
| | | G236D_G237F_S239D_S267V_H268D_V323A |
| 26399 | G236N_G237A | Template 1 (S325*A) + |
| | | G236D_G237F_S239D_S267V_H268D |
| 26417 | G236N_G237A | Template 1 (T326*A) + |
| | | G236D_G237F_S239D_S267V_H268D |
| 26428 | G236N_G237A | Template 1 (T326*D) + |
| | | G236D_G237F_S239D_S267V_H268D |
| 26429 | G236N_G237A | Template 1 (T326*E) + |
| | | G236D_G237F_S239D_S267V_H268D |
| 26422 | G236N_G237A | Template 1 (T326*F) + |
| | | G236D_G237F_S239D_S267V_H268D |
| 26432 | G236N_G237A | Template 1 (T326*H) + |
| | | G236D_G237F_S239D_S267V_H268D |
| 26420 | G236N_G237A | Template 1 (T326*I) + |
| | | G236D_G237F_S239D_S267V_H268D |
| 26419 | G236N_G237A | Template 1 (T326*L) + |
| | | G236D_G237F_S239D_S267V_H268D |
| 26427 | G236N_G237A | Template 1 (T326*N) + |
| | | G236D_G237F_S239D_S267V_H268D |
| 26426 | G236N_G237A | Template 1 (T326*Q) + |
| | | G236D_G237F_S239D_S267V_H268D |
| 26418 | G236N_G237A | Template 1 (T326*V) + |
| | | G236D_G237F_S239D_S267V_H268D |
| 26423 | G236N_G237A | Template 1 (T326*W) + |
| | | G236D_G237F_S239D_S267V_H268D |
| 26468 | G236N_G237A | Template 1 (F328*H) + |
| | | G236D_G237F_S239D_S267V_H268D |
| 26461 | G236N_G237A | Template 1 (F328*S) + |
| | | G236D_G237F_S239D_S267V_H268D |
| 26459 | G236N_G237A | Template 1 + (F328*Y) |
| | | G236D_G237F_S239D_S267V_H268D |
| 26470 | G236N_G237A | Template 1 (D329*G) + |
| | | G236D_G237F_S239D_S267V_H268D |
| 26474 | G236N_G237A | Template 1 (D329*I) + |
| | | G236D_G237F_S239D_S267V_H268D |
| 26473 | G236N_G237A | Template 1 (D329*L) + |
| | | G236D_G237F_S239D_S267V_H268D |
| 26488 | G236N_G237A | Template 1 (G330*A) + |
| | | G236D_G237F_S239D_S267V_H268D |
| 26500 | G236N_G237A | Template 1 (G330*D) + |
| | | G236D_G237F_S239D_S267V_H268D |
| 26501 | G236N_G237A | Template 1 (G330*E) + |
| | | G236D_G237F_S239D_S267V_H268D |
| 26504 | G236N_G237A | Template 1 (G330*H) + |
| | | G236D_G237F_S239D_S267V_H268D |
| 26503 | G236N_G237A | Template 1 (G330*K) + |
| | | G236D_G237F_S239D_S267V_H268D |
| 26502 | G236N_G237A | Template 1 (G330*R) + |
| | | G236D_G237F_S239D_S267V_H268D |
| 26530 | G236N_G237A | Template 1 (Y331*AF) + |
| | | G236D_G237F_S239D_S267V_H268D |
| 26531 | G236N_G237A | Template 1 (Y331*AW) + |
| | | G236D_G237F_S239D_S267V_H268D |
| 26546 | G236N_G237A | Template 1 (A331*BF) + |
| | | G236D_G237F_S239D_S267V_H268D |
| 26557 | G236N_G237A | Template 1 (A331*BH) + |
| | | G236D_G237F_S239D_S267V_H268D |
| 26556 | G236N_G237A | Template 1 (A331*BK) + |
| | | G236D_G237F_S239D_S267V_H268D |
| 26543 | G236N_G237A | Template 1 (A331*BL) + |
| | | G236D_G237F_S239D_S267V_H268D |
| 26563 | G236N_G237A | Template 1 + |
| | | G236D_G237F_S239D_S267V_H268D_I332F |
| 26561 | G236N_G237A | Template 1 + |
| | | G236D_G237F_S239D_S267V_H268D_I332L |
| 27374 | G236N_G237A | Template 1 (S325*A_A331*BN) + |
| | | G236D_G237F_S239D_S267V_H268D |
| 27372 | G236N_G237A | Template 1 (T326*H_F328*D) + |
| | | G236D_G237F_S239D_S267V_H268D |
| 27383 | G236N_G237A | Template 1 (T326*H_F328*E_D329*G_A331*BN) + |
| | | G236D_G237F_S239D_S267V_H268D |
| 27389 | G236N_G237A | Template 1 (T326*H_F328*E_D329*G_S325*A_A331*BN) + |
| | | G236D_G237F_S239D_S267V_H268D |
| 27365 | G236N_G237A | Template 1 (T326*H_F328*E_D329*G) + |
| | | G236D_G237F_S239D_S267V_H268D |
| 27385 | G236N_G237A | Template 1 (T326*H_A331*BN) + |
| | | G236D_G237F_S239D_S267V_H268D |
| 27391 | G236N_G237A | Template 1 (T326*H_S325*A_A331*BN) + |
| | | G236D_G237F_S239D_S267V_H268D |
| 27379 | G236N_G237A | Template 1 (T326*H_S325*A) + |
| | | G236D_G237F_S239D_S267V_H268D |
| 27373 | G236N_G237A | Template 1 (T326*H_F328*H) + |
| | | G236D_G237F_S239D_S267V_H268D |
| 27393 | G236N_G237A | Template 1 (T326*H_F328*H_D329*G) + |
| | | G236D_G237F_S239D_S267V_H268D |
| 27367 | G236N_G237A | Template 1 (T326*H_F328*N) + |
| | | G236D_G237F_S239D_S267V_H268D |
| 27368 | G236N_G237A | Template 1 (T326*H_F328*Q) + |
| | | G236D_G237F_S239D_S267V_H268D |
| 27384 | G236N_G237A | Template 1 (T326*H_F328*Q_D329*G_A331*BN) + |
| | | G236D_G237F_S239D_S267V_H268D |
| 27390 | G236N_G237A | Template 1 (T326*H_F328*Q_D329*G_S325*A_A331*BN) + |
| | | G236D_G237F_S239D_S267V_H268D |
| 27378 | G236N_G237A | Template 1 (T326*H_F328*Q_D329*G_S325*A) + |
| | | G236D_G237F_S239D_S267V_H268D |
| 27366 | G236N_G237A | Template 1 (T326*H_F328*Q_D329*G) + |
| | | G236D_G237F_S239D_S267V_H268D |
| 27381 | G236N_G237A | Template 1 (T326*H_F328*S_A331*BN) + |
| | | G236D_G237F_S239D_S267V_H268D |
| 27387 | G236N_G237A | Template 1 (T326*H_F328*S_S325*A_A331*BN) + |
| | | G236D_G237F_S239D_S267V_H268D |
| 27375 | G236N_G237A | Template 1 (T326*H_F328*S_S325*A) + |
| | | G236D_G237F_S239D_S267V_H268D |
| 27363 | G236N_G237A | Template 1 (T326*H_F328*S) + |
| | | G236D_G237F_S239D_S267V_H268D |
| 27371 | G236N_G237A | Template 1 (T326*H_F328*T) + |
| | | G236D_G237F_S239D_S267V_H268D |
| 27394 | G236N_G237A | Template 1 (F_328*H_D329*G) + |
| | | G236D_G237F_S239D_S267V_H268D |
| 27369 | G236N_G237A | Template 1 (F328*Q_D329*G) + |
| | | G236D_G237F_S239D_S267V_H268D |
| 27386 | G236N_G237A | Template 1 (F328*S_A331*BN) + |
| | | G236D_G237F_S239D_S267V_H268D |
| 27392 | G236N_G237A | Template 1 (F328*S_S325*A_A331*BN) + |
| | | G236D_G237F_S239D_S267V_H268D |
| 27370 | G236N_G237A | Template 1 (F328*S_D329*G) + |
| | | G236D_G237F_S239D_S267V_H268D |
| 27490 | G236N_G237A | Template 7 + |
| | | G236D_G237F_S239D_S267V_H268D |
| 27461 | G236N_G237A | Template 7 (E329*N_A331*BV_G325*F) + |
| | | G236D_G237F_S239D_S267V_H268D |
| 27453 | G236N_G237A | Template 7 (E329*N_A331*BY) + |
| | | G236D_G237F_S239D_S267V_H268D |
| 27463 | G236N_G237A | Template 7 (E328*H_E329*R_A331*BV_G325*F) + |
| | | G236D_G237F_S239D_S267V_H268D |
| 27455 | G236N_G237A | Template 7 (E328*H_E329*R_A331*BY) + |
| | | G236D_G237F_S239D_S267V_H268D |
| 27464 | G236N_G237A | Template 7 (E328*Q_E329*S_A331*BV_G325*F) + |
| | | G236D_G237F_S239D_S267V_H268D |
| 27456 | G236N_G237A | Template 7 (E328*Q_E329*S_A331*BY) + |
| | | G236D_G237F_S239D_S267V_H268D |
| 27462 | G236N_G237A | Template 7 (E328*T_E329*N_A331*BV_G325*F) + |
| | | G236D_G237F_S239D_S267V_H268D |
| 27454 | G236N_G237A | Template 7 (E328*T_E329*N_A331*BY) + |
| | | G236D_G237F_S239D_S267V_H268D |
| 27489 | G236N_G237A | Template 66 + |
| | | G236D_G237F_S239D_S267V_H268D |
| 27401 | G236N_G237A | Template 66 (Q328*E_N329*D_Q330D) + |
| | | G236D_G237F_S239D_S267V_H268D |
| 27403 | G236N_G237A | Template 66 (Q328*H_N329*D) + |
| | | G236D_G237F_S239D_S267V_H268D |
| 27405 | G236N_G237A | Template 66 (Q328*N_N329*D_Q330*D) + |
| | | G236D_G237F_S239D_S267V_H268D |
| 27404 | G236N_G237A | Template 66 (Q328*S_N329*T_Q330*D) + |
| | | G236D_G237F_S239D_S267V_H268D |
| 27408 | G236N_G237A | Template 66 (Q328*S_N329*T) + |
| | | G236D_G237F_S239D_S267V_H268D |
| 27406 | G236N_G237A | Template 66 (Q328*T_N329*D_Q330*D) + |
| | | G236D_G237F_S239D_S267V_H268D |
| 27410 | G236N_G237A | Template 66 (Q328*T_N329*S) + |
| | | G236D_G237F_S239D_S267V_H268D |
| 27411 | G236N_G237A | Template 66 (D327*N_Q328*H_N329*N_Q330*D) + |
| | | G236D_G237F_S239D_S267V_H268D |
| 27491 | G236N_G237A | Template 151 + |
| | | G236D_G237F_S239D_S267V_H268D |
| 27474 | G236N_G237A | Template 151 (E329*D_R331*S_Y331*BI) + |
| | | G236D_G237F_S239D_S267V_H268D |
| 27472 | G236N_G237A | Template 151 (E329*D_Y331*BI) + |
| | | G236D_G237F_S239D_S267V_H268D |
| 27471 | G236N_G237A | Template 151 (E328*H_E329*N_Y331*BI) + |
| | | G236D_G237F_S239D_S267V_H268D |
| 27466 | G236N_G237A | Template 151 (Y331*BI) + |
| | | G236D_G237F_S239D_S267V_H268D |

STRATEGY 2

| | | |
|---|---|---|
| 27294 | L234F_G236N_H268Q_A327G_A330K_P331S | G236D_S239D_V266L_S267A_H268D |
| 26593 | L234F_L235D_G236N_H268Q_A327G_A330K_P331S | G236D_S239D_V266L_S267A_H268D |
| 26663 | L234F_G236N_S267A_H268Q_A327G_A330K_P331S | G236D_S239D_V266L_S267A_H268D |
| 26847 | L234F_G236N_H268Q_A327G_A330T_P331S | G236D_S239D_V266L_S267A_H268D |
| 26940 | L234F_G236N_H268Q_A327G_A330K_P331S | G236D_G237D_S239D_V266L_S267A_H268D |
| 26931 | L234F_G236N_H268Q_A327G_A330K_P331S | G236D_G237L_S239D_V266L_S267A_H268D |

¹See footnote to Table 5A.

TABLE 5C
Exemplary Variants having Increased Selectivity for FcγRIIb

| Variant | Mutations^{1, 2} | |
|---|---|---|
| # | Chain A | Chain B |

STRATEGY ⅓

| | | |
|---|---|---|
| 31186 | G236N_G237D | Template 1 (D329*I) + |
| | | G236D_G237F_S239D_S267V_H268D |
| 31187 | L235F_G236N_G237A | Template 1 (D329*I) + |
| | | G236D_G237F_S239D_S267V_H268D |
| 31188 | L235F_G236N_G237A | Template 1 (G330*K) + |
| | | G236D_G237F_S239D_S267V_H268D |
| 31191 | G236N_G237D | Template 7 (E328*H_E329*R_A331*BY) + |
| | | G236D_G237F_S239D_S267V_H268D |
| 31213 | L235F_G236N_G237A | Template 1 (D329*I) + |
| | | G236D_G237F_S239D_S267V_H268D_I332L |
| 31274 | L235F_G236N_G237A_T250V_A287F | Template 1 (D329*I) + |
| | | G236D_G237F_S239D_T250V_S267V_H268D_A287F |
| 31275 | L235F_G236N_G237A_T250V_M428F | Template 1 (D329*I) + |
| | | G236D_G237F_S239D_T250V_S267V_H268D_M428F |
| 31276 | L235F_G236N_G237A_A287F_M428F | Template 1 (D329*I) + |
| | | G236D_G237F_S239D_S267V_H268D_A287F_M428F |
| 32210 | G236N_G237D | Template 1 (D329*I) + |
| | | G236D_G237F_S239D_S267V_H268D_I332L |
| 32211 | G236N_G237E | Template 1 (D329*I) + |
| | | G236D_G237F_S239D_S267V_H268D_I332L |
| 32212 | G236N_G237G | Template 1 (D329*I) + |
| | | G236D_G237F_S239D_S267V_H268D_I332L |
| 32226 | L235D_G236N_G237A | Template 1 (D329*I) + |
| | | G236D_G237F_S239D_S267V_H268D_I332L |
| 32227 | L235E_G236N_G237A | Template 1 (D329*I) + |
| | | G236D_G237F_S239D_S267V_H268D_I332L |
| 32230 | L235V_G236N_G237A | Template 1 (D329*I) + |
| | | G236D_G237F_S239D_S267V_H268D_I332L |
| 32231 | L235Y_G236N_G237A | Template 1 (D329*I) + |
| | | G236D_G237F_S239D_S267V_H268D_I332L |
| 32242 | G236N_G237A_S239P | Template 1 (D329*I) + |
| | | G236D_G237F_S239D_S267V_H268D_I332L |
| 32282 | L234D_G236N_G237A | Template 7 (E328*H_E329*R_A331*BY) + |
| | | G236D_G237F_S239D_S267V_H268D |
| 32284 | L235D_G236N_G237A | Template 7 (E328*H_E329*R_A331*BY) + |
| | | G236D_G237F_S239D_S267V_H268D |
| 32287 | G236N_G237A_S239G | Template 7 (E328*H_E329*R_A331*BY) + |
| | | G236D_G237F_S239D_S267V_H268D |
| 32288 | G236N_G237A_S239H | Template 7 (E328*H_E329*R_A331*BY) + |
| | | G236D_G237F_S239D_S267V_H268D |
| 32296 | G236N_G237E | Template 7 (E328*H_E329*R_A331*BY) + |
| | | G236D_G237F_S239D_S267V_H268D |

STRATEGY 2

| | | |
|---|---|---|
| 31190 | L234F_L235D_G236N_H268Q_A327G_A330K_P331S | G236D_G237L_S239D_V266L_S267A_H268D |
| 31256 | L234F_G236N_H268Q_A327G_P329I_A330K_P331S | G236D_G237D_S239D_V266L_S267A_H268D |
| 32274 | L234F_G236N_H268Q_A327G_P329A_A330K_P331S | G236D_G237L_S239D_V266L_S267A_H268D |

STRATEGY ⅓ + STRATEGY 2

| | | |
|---|---|---|
| 31192 | L234F_L235D_G236N_H268Q_A327G_A330K_P331S | Template 1 (D329*I) + |
| | | G236D_G237F_S239D_S267V_H268D |
| 32292 | L234F_L235D_G236N_H268Q_A327G_A330K_P331S | Template 1 (D329*I) + |
| | | G236D_G237F_S239D_S267V_H268D_I332L |
| 32293 | L234F_G236N_S267A_H268Q_A327G_A330K_P331S | Template 1 (D329*I) + |
| | | G236D_G237F_S239D_S267V_H268D_I332L |
| 32294 | L234F_G236N_H268Q_A327G_A330T_P331S | Template 1 (D329*I) + |
| | | G236D_G237F_S239D_S267V_H268D_I332L |
| 32295 | L234F_G236N_H268Q_A327G_P329I_A330K_P331S | Template 1 (D329*I) + |
| | | G236D_G237F_S239D_S267V_H268D_I332L |

¹See footnote to Table 5A
²Template 1 (D329*I) has the sequence set forth in SEQ ID NO: 47, Template 1 (D330*K) has the sequence set forth in SEQ ID NO: 68, and Template 7 (E328*H_E329*R_A331*BY) has the sequence set forth in SEQ ID NO: 73.

### Stability-Enhancing Mutations

In certain embodiments, the heterodimeric Fc variant may further comprise one or more mutations that increase the thermostability of the variant (“stability-enhancing mutations”). Inclusion of one or more stability-enhancing mutations may be particularly useful when the heterodimeric Fc variant exhibits a low CH2 domain melting temperature (Tm) as compared to the Tm for wild-type IgG1 CH2 domain, which is typically between about 69° C. and about 73° C. as measured by differential scanning calorimetry (DSC).

As described herein, the following mutations were shown to increase the thermostability of heterodimeric Fc variants while retaining the FcγRIIb selectivity: A287F, T250V, L309Q, M428F, A287F/M428F, A287F/T250V, M428F/T250V and T250V/L309Q. Accordingly, in certain embodiments, the heterodimeric Fc variant may further comprise one or more stability-enhancing mutations selected from A287F, T250V, L309Q and M428F. In some embodiments, the heterodimeric Fc variants may comprise two stability-enhancing mutations selected from A287F, T250V, L309Q and M428F. In some embodiments, the heterodimeric Fc variant comprises one stability-enhancing mutation selected from: A287F, T250V, L309Q and M428F. In some embodiments, the heterodimeric Fc variant comprises two stability-enhancing mutations selected from: A287F/M428F, A287F/T250V, M428F/T250V and T250V/L309Q.

When the heterodimeric Fc variant comprises stability-enhancing mutation or mutations as described above, the mutation(s) are introduced symmetrically into the Fc, that is, the mutation(s) are present in both the first Fc polypeptide and the second Fc polypeptide of the heterodimeric Fc variant.

Other mutations that are known to increase the thermostability of an Fc and may be included in the heterodimeric Fc variant in some embodiments include those described in U.S. Patent Application Publication No. 2015/0210763.

### CH3 Domain Mutations

In certain embodiments, the heterodimeric Fc variants described herein comprise a modified CH3 domain which comprises one or more asymmetric amino acid mutations that promote formation of the heterodimeric Fc over formation of a homodimeric Fc.

Various amino acid mutations that may be made to the CH3 domain of an Fc in order to promote formation of a heterodimeric Fc are known in the art and include, for example, those described in International Patent Application Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Patent Application Publication Nos. WO 2012/058768 and WO 2013/063702.

In certain embodiments, the heterodimeric Fc variant comprises a modified CH3 domain comprising mutations based on the “knobs into holes” approach. In some embodiments, the heterodimeric Fc variant comprises a modified CH3 domain in which one Fc polypeptide comprises the amino acid mutations Y349C, T366S, L368A and Y407V, and the other Fc polypeptide comprises the amino acid mutations S354C and T366W.

In certain embodiments, the heterodimeric Fc variant comprises a modified CH3 domain comprising mutations based on the “electrostatic steering” approach. In some embodiments, the heterodimeric Fc variant comprises a modified CH3 domain in which one Fc polypeptide comprises the amino acid mutations K392D and K409D, and the other Fc polypeptide comprises the amino acid mutations E356K and D399K.

In certain embodiments, the heterodimeric Fc variant comprises a modified CH3 domain as described in International Patent Application Publication No. WO 2012/058768 or WO 2013/063702.

In certain embodiments, the heterodimeric Fc variant comprises a modified CH3 domain in which one Fc polypeptide comprises amino acid mutations at positions F405 and Y407, and the other Fc polypeptide comprises amino acid mutations at positions T366 and T394. In some embodiments, the amino acid mutation at position F405 is F405A, F405S, F405T or F405V. In some embodiments, the amino acid mutation at position Y407 is Y407I or Y407V. In some embodiments, the amino acid mutation at position T366 is T366I, T366L or T366M. In some embodiments, the amino acid mutation at position T366 is T366I or T366L. In some embodiments, the amino acid mutation at position T394 is T394W.

In some embodiments, one Fc polypeptide comprises amino acid mutations at positions F405 and Y407 as described above, and further includes an amino acid mutation at position L351. In some embodiments, the amino acid mutation at position L351 is L351Y.

In some embodiments, one Fc polypeptide comprises amino acid mutations at positions T366 and T394 as described above, and further includes an amino acid mutation at position K392. In some embodiments, the amino acid mutation at position K392 is K392F, K392L or K392M. In some embodiments, the amino acid mutation at position K392 is K392L or K392M.

In some embodiments, the heterodimeric Fc variant comprises a modified CH3 domain in which one Fc polypeptide comprises amino acid mutations at positions F405 and Y407, and optionally further comprises an amino acid mutation at position L351, and the other Fc polypeptide comprises amino acid mutations at positions T366 and T394, and optionally further comprises an amino acid mutation at position K392, as described above, and one or both of the Fc polypeptides further comprises the amino acid mutation T350V.

In certain embodiments, the heterodimeric Fc variant comprises a modified CH3 domain in which one Fc polypeptide comprises the amino acid mutation F405A, F405S, F405T or F405V together with the amino acid mutation Y407I or Y407V, and optionally further includes the amino acid mutation L351Y, and the other Fc polypeptide comprises the amino acid mutation T366I or T366L, together with the amino acid mutation T394W, and optionally further includes the amino acid mutation K392L or K392M. In some embodiments, one or both of the Fc polypeptides further comprises the amino acid mutation T350V. In some embodiments, both Fc polypeptides further comprise the amino acid mutation T350V.

In certain embodiments, the heterodimeric Fc variant comprises a modified CH3 domain in which the first Fc polypeptide comprises amino acid modifications at positions F405 and Y407, and optionally further comprises an amino acid modification at position L351, and the second Fc polypeptide comprises amino acid modifications at positions T366 and T394, and optionally further comprises an amino acid modification at position K392, as described above, and the first Fc polypeptide further comprises an amino acid modification at one or both of positions S400 or Q347 and/or the second Fc polypeptide further comprises an amino acid modification at one or both of positions K360 or N390, where the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D.

In certain embodiments, the heterodimeric Fc variant comprises a modified CH3 domain comprising the amino acid modifications as set forth for any one of Variant 1, Variant 2, Variant 3, Variant 4 or Variant 5 in Table 6.

TABLE 6
Modified CH3 Domains

| Variant # | Chain | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

### Assays to Test Activity

The heterodimeric Fc variants of the present disclosure have increased selectivity for FcγRIIb as compared to the parental Fc region. By “increased selectivity for FcγRIIb” it is meant that the heterodimeric Fc variant shows a greater improvement in affinity for FcγRIIb relative to any improvement in affinity for FcγRIIaR, as compared to the parental Fc region. In certain embodiments, the heterodimeric Fc variant shows a greater affinity for FcγRIIb relative to its affinity for FcγRIIaR as compared to the parental Fc region.

Candidate heterodimeric Fc variants may be tested for FcγRIIb selectivity using standard methods known in the art. For example, the binding affinity of a heterodimeric Fc variant to each of the Fcγ receptors may be measured by surface plasmon resonance (SPR), SPR imaging (SPRi), bio-layer interferometry (BLI), ELISA, Kinetic Exclusion Assay (KinExA®) or Meso Scale Discovery™ (MSD™)-based methods (see, for example, Current Protocols in Immunology: Ligand-Receptor Interactions in the Immune System, Eds. J. Coligan et al., 2018 & updates, Wiley Inc., Hoboken, NJ; Yang et al., 2016, Analytical Biochem, 508:78-96) and compared with the binding affinity of the parental Fc variant to the Fcγ receptors. Typically, binding affinity is expressed in terms of the dissociation constant (K_{D}) for binding of the heterodimeric Fc variant to the Fcγ receptor.

Selectivity may be expressed as a fold increase in FcγRIIb selectivity with respect to the parental Fc region. In the context of the present disclosure, the fold difference in FcγRIIb selectivity is calculated as follows. First, the K_{D} for binding to FcγRIIb for each of the heterodimeric Fc variant and the parental Fc region is determined and the fold difference in FcγRIIb affinity for the variant is determined according to equation [4]:

K_{D} FcγRIIb (parental)/K_{D} FcγRIIb (variant)=Fold Difference in FcγRIIb Affinity [4]

The K_{D} for binding to FcγRIIaR for each of the heterodimeric Fc variant and the parental Fc region is also determined and the fold difference in FcγRIIaR affinity for the variant is determined according to equation [5]:

K_{D} FcγRIIaR (parental)/K_{D} FcγRIIaR (variant)=Fold Difference in FcγRIIaR Affinity [5]

The fold difference in FcγRIIb selectivity for the heterodimeric Fc variant with respect to the parental Fc region may then be calculated according to equation [6]:

Fold Difference in FcγRIIb Affinity/Fold Difference in FcγRIIaR Affinity=Fold Difference in FcγRIIb Selectivity [6]

where a result >1 indicates an increase in FcγRIIb selectivity with respect to the parental Fc region, and a result <1 indicates a decrease in FcγRIIb selectivity with respect to the parental Fc region.

In certain embodiments, the heterodimeric Fc variant has selectivity for FcγRIIb that is increased by at least 1.5-fold over the parental Fc region. In some embodiments, the heterodimeric Fc variant has selectivity for FcγRIIb that is increased by at least 2-fold over the parental Fc region. In some embodiments, the heterodimeric Fc variant has selectivity for FcγRIIb that is increased by at least 3-fold over the parental Fc region, for example, at least 4-fold, at least 5-fold, at least 6-fold, at least 7-fold, at least 8-fold or at least 9-fold over the parental Fc region.

In some embodiments, the heterodimeric Fc variant has selectivity for FcγRIIb that is increased by at least 10-fold over the parental Fc region. In some embodiments, the heterodimeric Fc variant has selectivity for FcγRIIb that is increased by at least 15-fold over the parental Fc region, at least 20-fold over the parental Fc region, at least 25-fold over the parental Fc region, at least 30-fold over the parental Fc region, at least 35-fold over the parental Fc region, at least 40-fold over the parental Fc region, or at least 50-fold over the parental Fc region.

In certain embodiments, the heterodimeric Fc variant also has increased affinity for FcγRIIb as compared to the parental Fc region. By “increased affinity for FcγRIIb” it is meant that the heterodimeric Fc variant shows an increased affinity for FcγRIIb as compared to the affinity of the parental Fc for FcγRIIb. Affinity may be measured, for example, by determining the dissociation constant (K_{D}) by standard techniques as described above.

The increased affinity of a heterodimeric Fc variant for FcγRIIb may be expressed as the fold increase over the affinity of the parental Fc region. In the context of the present disclosure, the fold increase may be calculated as outlined above. Specifically, the K_{D} for binding to FcγRIIb for each of the heterodimeric Fc variant and the parental Fc region is determined and the fold difference in FcγRIIb affinity for the variant is determined according to equation [4]:

K_{D} FcγRIIb (parental)/K_{D} FcγRIIb (variant)=Fold Difference in FcγRIIb Affinity [4]

where a result >1 indicates an increase in FcγRIIb affinity with respect to the parental Fc region, and a result <1 indicates a decrease in FcγRIIb affinity with respect to the parental Fc region.

In certain embodiments, the heterodimeric Fc variant has an affinity for FcγRIIb that is increased by at least 5-fold over the parental Fc region. In some embodiments, the heterodimeric Fc variant has an affinity for FcγRIIb that is increased by at least 10-fold over the parental Fc region, for example, at least 15-fold, at least 20-fold, or at least 25-fold over the parental Fc region. In some embodiments, the heterodimeric Fc variant has an affinity for FcγRIIb that is increased by at least 30-fold over the parental Fc region, at least 40-fold over the parental Fc region, or at least 50-fold over the parental Fc region. In some embodiments, the heterodimeric Fc variant has an affinity for FcγRIIb that is increased by at least 100-fold over the parental Fc region.

In certain embodiments, the heterodimeric Fc variant has selectivity for FcγRIIb that is increased by at least 5-fold over the parental Fc region and an affinity for FcγRIIb that is increased by at least 5-fold over the parental Fc region. In some embodiments, the heterodimeric Fc variant has selectivity for FcγRIIb that is increased by at least 5-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the heterodimeric Fc variant has selectivity for FcγRIIb that is increased by at least 10-fold over the parental Fc region, and an affinity for FcγRIIb that is increased by at least 5-fold over the parental Fc region. In some embodiments, the heterodimeric Fc variant has selectivity for FcγRIIb that is increased by at least 10-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the heterodimeric Fc variant has selectivity for FcγRIIb that is increased by at least 20-fold over the parental Fc region, and an affinity for FcγRIIb that is increased by at least 5-fold over the parental Fc region. In some embodiments, the heterodimeric Fc variant has selectivity for FcγRIIb that is increased by at least 20-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the heterodimeric Fc variant has selectivity for FcγRIIb that is increased by at least 30-fold over the parental Fc region, and an affinity for FcγRIIb that is increased by at least 5-fold over the parental Fc region. In some embodiments, the heterodimeric Fc variant has selectivity for FcγRIIb that is increased by at least 30-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the heterodimeric Fc variant has selectivity for FcγRIIb that is increased by at least 40-fold over the parental Fc region, and an affinity for FcγRIIb that is increased by at least 5-fold over the parental Fc region. In some embodiments, the heterodimeric Fc variant has selectivity for FcγRIIb that is increased by at least 40-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the heterodimeric Fc variant has selectivity for FcγRIIb that is increased by at least 50-fold over the parental Fc region, and an affinity for FcγRIIb that is increased by at least 5-fold over the parental Fc region. In some embodiments, the heterodimeric Fc variant has selectivity for FcγRIIb that is increased by at least 50-fold over the parental Fc region and an affinity for FcγRIIb that is increased by at least 10-fold, at least 15-fold, at least 20-fold, at least 25-fold, at least 30-fold, at least 40-fold, or at least 50-fold over the parental Fc region.

In certain embodiments, the K_{D} values used to determine the FcγRIIb affinity and selectivity of the heterodimeric Fc variant are determined by SPR. In SPR assays to assess antibody Fc-FcγR binding, various formats may be employed. For example, the assay may employ receptor immobilized on the biosensor chip with antibody in solution flowed over the chip, or the assay may employ antibody immobilized on the biosensor chip with receptor in solution flowed over the chip, or the assay may employ target antigen immobilized on the biosensor chip with antibody in solution flowed over the chip first followed by receptor in solution. In certain embodiments, the K_{D} values used to determine the FcγRIIb affinity and selectivity of the heterodimeric Fc variant are determined by SPR using a format in which target antigen is immobilized on the biosensor chip with antibody in solution flowed over the chip first followed by receptor in solution.

Other assays may optionally be conducted using standard techniques in order to further characterize the heterodimeric Fc variants. For example, the heterodimeric Fc variants may be assessed for purity, FcRn binding, aggregation, thermal stability and/or C1q binding. Purity and aggregation may be assessed, for example, by liquid chromatography-mass spectrometry (LC-MS) or size-exclusion chromatography (SEC). FcRn binding may be assessed, for example, using standard techniques such as those outlined above for FcγR binding. Thermal stability may be assessed, for example, by circular dichroism (CD), differential scanning calorimetry (DSC) or differential scanning fluorimetry (DSF). C1q binding may be assessed, for example, by ELISA or surface plasmon resonance (SPR). Exemplary methods for assessing various properties of the heterodimeric Fc variants are described in the Examples provided herein.

### Polypeptides

Certain embodiments of the present disclosure relate to polypeptides comprising a heterodimeric Fc variant as described herein. Typically, the polypeptides comprise one or more additional proteinaceous moieties fused to the heterodimeric Fc variant or covalently attached to the heterodimeric Fc variant, for example, by means of a linker. For example, the polypeptide may be an Fc fusion protein or an antibody or antibody fragment. Examples of proteinaceous moieties that may be fused or attached to the heterodimeric Fc variant include, but are not limited to, antigen-binding domains, ligands, receptors, receptor fragments, cytokines and antigens.

When the polypeptides comprise more than one additional proteinaceous moiety, the moieties may be the same or they may be different. The one or more additional proteinaceous moieties may be fused or covalently attached at the N-terminus, the C-terminus or both the N-terminus and the C-terminus of one or both of the Fc polypeptides. In some embodiments, the polypeptides comprise one or more additional proteinaceous moieties fused or covalently attached to the N-terminus of one or both of the Fc polypeptides. In some embodiments, the polypeptides comprise one additional proteinaceous moiety fused or covalently attached to the N-terminus of one of the Fc polypeptides. In some embodiments, the polypeptides comprise two additional proteinaceous moieties, one moiety fused or covalently attached to the N-terminus of the first Fc polypeptide and the other moiety fused or covalently attached to the N-terminus of the second Fc polypeptide. In some embodiments, two additional proteinaceous moieties comprised by the polypeptides may be linked in tandem.

In some embodiments, the polypeptides comprise a heterodimeric Fc variant fused or covalently attached to one or more proteinaceous moieties that are antigen-binding domains. In some embodiments, the polypeptides comprise a heterodimeric Fc variant and one or more antigen-binding domains. In some embodiments, the polypeptides comprise a heterodimeric Fc variant and two or more antigen-binding domains, for example, 2, 3, 4, 5, 6, 7 or 8 antigen-binding domains. When the polypeptide comprises a heterodimeric Fc variant and two or more antigen-binding domains, the antigen-binding domains may bind the same antigen or they may bind different antigens.

In some embodiments, the polypeptides comprise a heterodimeric Fc variant fused or covalently attached to one or more proteinaceous moieties that are antigen-binding domains and to one or more other proteinaceous moieties. In some embodiments, the polypeptides comprise a heterodimeric Fc variant fused or covalently attached to an antigen-binding domain and to one or more other proteinaceous moieties. Examples of other proteinaceous moieties in this context include, but are not limited to, receptors, receptor fragments (such as extracellular portions), ligands and cytokines.

In some embodiments, the polypeptide may be an antibody or an antibody fragment in which at least one of the one or more proteinaceous moieties is an antigen-binding domain. For example, the antigen-binding domain may be a Fab fragment, Fv fragment, single-chain Fv fragment (scFv) or single domain antibody (sdAb). In some embodiments, the polypeptide may be a monospecific antibody. In some embodiments, the polypeptide may be a monospecific antibody comprising one antigen-binding domain. In some embodiments, the polypeptide may be a monospecific antibody comprising two antigen-binding domains. In some embodiments, the polypeptide may be a monospecific antibody comprising more than two antigen-binding domains. In some embodiments, the polypeptide may be a bispecific or multispecific antibody comprising a heterodimeric Fc variant and two or more antigen-binding domains, in which two or more antigen-binding domains bind to different antigens.

In some embodiments, the polypeptide may be an agonistic antibody. It has been reported that the agonistic activity of antibodies against members of the TNF receptor family (such as CD40, DR4, DR5, CD30 and CD137) requires interaction with FcγRIIb (see, for example, White, et al., 2011, J Immunol., 187:1754-1763). Accordingly, in some embodiments, the heterodimeric Fc variants may be used as the Fc region of an agonistic antibody against a member of the TNF receptor family in order to enhance the agonistic activity of the antibody. Certain embodiments of the present disclosure relate to agonistic antibodies comprising a heterodimeric Fc variant as described herein, where the agonistic antibody comprises one or more antigen-binding domains that bind to a member of the TNF receptor family.

In some embodiments, the polypeptides comprise a heterodimeric Fc variant and one or more antigen-binding domains, where at least one of the antigen-binding domains binds to a tumour-associated antigen or tumour-specific antigen.

In some embodiments, the polypeptides may be Fc fusion proteins in which the one or more proteinaceous moieties may be, for example, a ligand for a cell-surface receptor, a soluble fragment of a cell-surface receptor, a biologically active peptide, a cytokine, a growth factor, a hormone or an enzyme. Examples of proteinaceous moieties that may be included in an Fc fusion protein as described herein include, but are not limited to, ligands, such as tumor necrosis factor (TNF), PD-L1, ICOS-L, VEGF and LFA-3; extracellular ligand-binding portions of cell-surface receptors, such as TNFR, PD-1, CTLA-4, ICOS, VEGFR and IL-1R; biologically active peptides, such as thrombopoietin binding peptide, hormones such as erythropoietin (Epo), cytokines such as interferon α or interferon β, or enzymes such as Factor IX.

### Preparation of Heterodimeric Fc Variants

The heterodimeric Fc variants described herein and polypeptides comprising a heterodimeric Fc variant as described herein may be prepared using standard recombinant methods. Recombinant production of the heterodimeric Fc variants and polypeptides generally involves synthesizing one or more polynucleotides encoding the heterodimeric Fc variant or polypeptide, cloning the one or more polynucleotides into an appropriate vector or vectors, and introducing the vector(s) into a suitable host cell for expression of the heterodimeric Fc variant or polypeptide. Recombinant production of proteins is well-known in the art and may be achieved using standard techniques as described, for example, in Sambrook et al., Molecular Cloning: A Laboratory Manual, 3rd ed., Cold Spring Harbor Laboratory Press, Cold Spring Harbor, N Y (2001); Ausubel et al., Current Protocols in Molecular Biology, (1987 & updates), John Wiley & Sons, New York, NY; and Harlow and Lane, Antibodies: A Laboratory Manual, Cold Spring Harbor Laboratory Press, Cold Spring Harbor, NY (1990).

Certain embodiments of the present disclosure thus relate to an isolated polynucleotide or set of polynucleotides encoding a heterodimeric Fc variant as described herein or polypeptide comprising a heterodimeric Fc variant as described herein. A polynucleotide in this context may encode all or part of a heterodimeric Fc variant or polypeptide.

The terms “nucleic acid,” “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, isolated DNA, isolated RNA, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The one or more polynucleotides encoding the heterodimeric Fc variant or polypeptide may be inserted into a suitable expression vector, either directly or after one or more subcloning steps, using standard ligation techniques. Examples of suitable vectors include, but are not limited to, plasmids, phagemids, cosmids, bacteriophage, baculoviruses, retroviruses or DNA viruses. The vector is typically selected to be functional in the particular host cell that will be employed, i.e. the vector is compatible with the host cell machinery, permitting amplification and/or expression of the polynucleotide(s). Selection of appropriate vector and host cell combinations in this regard is well within the ordinary skills of a worker in the art.

Certain embodiments of the present disclosure thus relate to vectors (such as expression vectors) comprising one or more polynucleotides encoding a heterodimeric Fc variant or polypeptide comprising a heterodimeric Fc variant. The polynucleotide(s) may be comprised by a single vector or by more than one vector. In some embodiments, the polynucleotides are comprised by a multicistronic vector.

Typically, expression vectors will contain one or more regulatory elements for plasmid maintenance and for cloning and expression of exogenous polynucleotide sequences. Examples of such regulatory elements include promoters, enhancer sequences, origins of replication, transcriptional termination sequences, donor and acceptor splice sites, leader sequences for polypeptide secretion, ribosome binding sites, polyadenylation sequences, polylinker regions for inserting the polynucleotide encoding the polypeptide to be expressed, and selectable markers.

Regulatory elements may be homologous (i.e. from the same species and/or strain as the host cell), heterologous (i.e. from a species other than the host cell species or strain), hybrid (i.e. a combination of sequences from more than one source) or synthetic. As such, the source of a regulatory element may be any prokaryotic or eukaryotic organism provided that the sequence is functional in, and can be activated by, the machinery of the host cell being employed.

Optionally, the vector may contain a “tag”-encoding sequence, i.e. a nucleic acid sequence located at the 5′ or 3′ end of the coding sequence that encodes a heterologous peptide sequence, such as a polyHis (for example, 6×His), FLAG®, HA (hemaglutinin influenza virus), myc, metal-affinity, avidin/streptavidin, glutathione-S-transferase (GST) or biotin tag. This tag typically remains fused to the expressed protein and can serve as a means for affinity purification or detection of the protein. Optionally, the tag can subsequently be removed from the purified protein by various means such as using certain peptidases for cleavage.

Various expression vectors are readily available from commercial sources. Alternatively, when a commercial vector containing all the desired regulatory elements is not available, an expression vector may be constructed using a commercially available vector as a starting vector. Where one or more of the desired regulatory elements are not already present in the vector, they may be individually obtained and ligated into the vector. Methods for obtaining various regulatory elements are well known to one skilled in the art.

Once the expression vector including the polynucleotide(s) encoding the heterodimeric Fc variant or polypeptide has been constructed, the vector may be inserted into a suitable host cell for amplification and/or protein expression. The transformation of an expression vector into a selected host cell may be accomplished by well-known methods including transfection, infection, calcium phosphate co-precipitation, electroporation, microinjection, lipofection, DEAE-dextran mediated transfection, and other known techniques. The method selected will in part be a function of the type of host cell to be used. These methods and other suitable methods are well known to the skilled person (see, for example, Sambrook, et al., ibid.).

A host cell, when cultured under appropriate conditions, expresses the protein encoded by the vector and the protein can subsequently be collected from the culture medium (if the host cell secretes the protein) or directly from the host cell producing it (if the protein is not secreted). The host cell may be prokaryotic (for example, a bacterial cell) or eukaryotic (for example, a yeast, fungi, plant or mammalian cell). The selection of an appropriate host cell can be readily made by the skilled person taking into account various factors, such as desired expression levels, polypeptide modifications that are desirable or necessary for activity (such as glycosylation or phosphorylation) and ease of folding into a biologically active molecule.

Certain embodiments of the present disclosure thus relate to host cells comprising polynucleotide(s) or one or more vectors comprising the polynucleotide(s). In certain embodiments, the host cell is a eukaryotic cell.

For example, eukaryotic microbes such as filamentous fungi or yeast may be employed as host cells, including fungi and yeast strains whose glycosylation pathways have been “humanized” (see, for example, Gerngross, (2004), Nat. Biotech., 22:1409-1414, and Li et al., (2006), Nat. Biotech., 24:210-215). Plant cells may also be utilized as host cells (see, for example, U.S. Pat. Nos. 5,959,177; 6,040,498; 6,420,548; 7,125,978 and 6,417,429, describing PLANTIBODIES™ technology).

In some embodiments, the host cell is a mammalian cell. Various mammalian cell lines may be used as host cells. Examples of useful mammalian host cell lines include, but are not limited to, monkey kidney CV1 line transformed by SV40 (COS-7), human embryonic kidney line 293 (HEK293 cells as described, for example, in Graham, et al., (1977), J. Gen Virol., 36:59), baby hamster kidney cells (BHK), mouse sertoli cells (TM4 cells as described, for example, in Mather, (1980), Biol. Reprod., 23:243-251), monkey kidney cells (CV1), African green monkey kidney cells (VERO-76), human cervical carcinoma cells (HeLa), canine kidney cells (MDCK), buffalo rat liver cells (BRL 3A), human lung cells (W138), human liver cells (Hep G2), mouse mammary tumour (MMT 060562), TRI cells (as described, for example, in Mather, et al., 1982, Annals N.Y. Acad. Sci., 383:44-68), MRC 5 cells, FS4 cells, Chinese hamster ovary (CHO) cells (including DHFR⁻ CHO cells as described in Urlaub, et al., 1980, Proc. Natl. Acad. Sci. USA, 77:4216) and myeloma cell lines (such as Y0, NS0 and Sp2/0). See also, Yazaki and Wu, 2003, Methods in Molecular Biology, Vol. 248, pp. 255-268 (B. K. C. Lo, ed., Humana Press, Totowa, N.J.).

Certain embodiments of the present disclosure relate to methods of preparing a heterodimeric Fc variant as described herein or a polypeptide comprising a heterodimeric Fc variant as described herein, comprising transfecting a host cell with one or more polynucleotides encoding the heterodimeric Fc variant or polypeptide, for example as one or more vectors comprising the polynucleotide(s), and culturing the host cell under conditions suitable for expression of the encoded heterodimeric Fc variant or polypeptide.

Typically, the heterodimeric Fc variant or polypeptide is isolated from the host cell after expression and may optionally be purified. Methods for isolating and purifying expressed proteins are well-known in the art. Standard purification methods include, for example, chromatographic techniques, such ion exchange, hydrophobic interaction, affinity, sizing, gel filtration or reverse-phase, which may be carried out at atmospheric pressure or at medium or high pressure using systems such as FPLC, MPLC and HPLC. Other purification methods include electrophoretic, immunological, precipitation, dialysis, and chromatofocusing techniques. Ultrafiltration and diafiltration techniques, in conjunction with protein concentration, may also be useful.

A variety of natural proteins are known in the art to bind Fc regions or other regions of antibodies, and these proteins can therefore be used in the purification of Fc-containing proteins. For example, the bacterial proteins A and G bind to the Fc region. Likewise, the bacterial protein L binds to the Fab region of some antibodies. Purification can often be enabled by a particular fusion partner or affinity tag as described above. For example, antibodies may be purified using glutathione resin if a GST fusion is employed, Ni⁺² affinity chromatography if a His-tag is employed, or immobilized anti-flag antibody if a FLAG-tag is used. Examples of useful purification techniques are described in Harlow and Lane, Antibodies: A Laboratory Manual, Cold Spring Harbor Laboratory Press, Cold Spring Harbor, NY (1990), and Protein Purification: Principles and Practice, 3^{rd} Ed., Scopes, Springer-Verlag, NY (1994).

### Methods of Use

Certain embodiments of the present disclosure relate to the therapeutic use of the heterodimeric Fc variants described herein and polypeptides comprising the heterodimeric Fc variants.

For example, in some embodiments, the heterodimeric Fc variants and polypeptides described herein which selectively activate FcγRIIb may be used to suppress the activation of B cells, mast cells, dendritic cells, and/or basophils. Activation of B cells includes proliferation, IgE production, IgM production and IgA production. Certain embodiments of the present disclosure relate to polypeptides comprising a heterodimeric Fc variant and one or more antigen-binding domains that bind a molecule expressed on the surface of B cells, such as CD19 or CD79b. Such polypeptides may be particularly useful in inhibiting B cell activation by cross-linking FcγRIIb with the B cell.

Certain embodiments relate to the use of the heterodimeric Fc variants and polypeptides described herein in the treatment of inflammatory diseases and disorders. In some embodiments, the heterodimeric Fc variants and polypeptides described herein may be used in the treatment of autoimmune diseases or disorders. One skilled in the art will appreciate that some diseases and disorders may be characterized as both inflammatory and autoimmune, thus these two categories are not mutually exclusive. Examples of diseases and disorders that may be characterized as inflammatory and/or autoimmune include, but are not limited to, Addison's disease, ankylosing spondylitis, autoimmune vasculitis, celiac disease, diabetes Type I, diabetes Type II, gout, gouty arthritis, Graves' disease, Hashimoto's thyroiditis, inflammatory bowel disease (IBD), multiple sclerosis, myasthenia gravis, myositis, pernicious anemia, psoriasis, psoriatic arthritis, rheumatoid arthritis, scleroderma, Sjögren's syndrome and systemic lupus erythematosus (SLE).

Certain embodiments relate to the use of the heterodimeric Fc variants and polypeptides disclosed herein in the treatment of cancer. In this context, treatment with the heterodimeric Fc variant or polypeptide may result in one or more of a reduction in the size of a tumour, the slowing or prevention of an increase in the size of a tumour, an increase in the disease-free survival time between the disappearance or removal of a tumour and its reappearance, prevention of a subsequent occurrence of a tumour (for example, metastasis), an increase in the time to progression, reduction of one or more adverse symptom associated with a tumour, or an increase in the overall survival time of a subject having cancer.

Examples of cancers which may be treated or stabilized in accordance with certain embodiments include haematologic cancers (including leukaemias, myelomas and lymphomas), carcinomas (including adenocarcinomas and squamous cell carcinomas), melanomas and sarcomas. Carcinomas and sarcomas are also frequently referred to as “solid tumours.” Examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, are also often considered to be solid tumours.

As described above, it is known that increasing FcγRIIb binding of an agonistic antibody enhances the agonistic activity of the antibody, which in turn will enhance the anti-tumour effect of the antibody. Accordingly, some embodiments of the present disclosure relate to methods of treating cancer with a polypeptide that is an agonistic antibody against a receptor of the TNF receptor family and comprises a heterodimeric Fc variant as described herein.

### Pharmaceutical Compositions

For therapeutic use, the heterodimeric Fc variants and polypeptides may be provided in the form of compositions which comprise the heterodimeric Fc variant or polypeptide and a pharmaceutically acceptable carrier or diluent. The compositions may be prepared by known procedures using well-known and readily available ingredients and may be formulated for administration to a subject by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal or intrathecal routes.

The composition will typically be formulated in a format suitable for administration to the subject by the chosen route, for example, as a syrup, elixir, tablet, troche, lozenge, hard or soft capsule, pill, suppository, oily or aqueous suspension, dispersible powder or granule, emulsion, injectable or solution. Compositions may be provided as unit dosage formulations.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions may be in the form of a sterile injectable aqueous or oleaginous solution or suspension. Such suspensions may be formulated using suitable dispersing or wetting agents and/or suspending agents that are known in the art. The sterile injectable solution or suspension may comprise the heterodimeric Fc variant or polypeptide in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents as known in the art may also be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### EMBODIMENTS

Exemplary non-limiting embodiments of the present disclosure include the following:

1. A heterodimeric Fc variant comprising a first Fc polypeptide and a second Fc polypeptide, the heterodimeric Fc variant having increased selectivity of binding to FcγRIIb as compared to a parental Fc region,
wherein one of the Fc polypeptides comprises a replacement of all or a part of a natural loop in the CH2 domain of the Fc polypeptide with an alternative amino acid sequence such that the natural loop is extended in length and at least one of the amino acid residues of the alternative amino acid sequence is within a heavy atom to heavy atom distance of 3 Å of a target amino acid residue in FcγRIIb when the heterodimeric Fc variant is bound by FcγRIIb, and
wherein the heterodimeric Fc variant is a variant of an immunoglobulin G (IgG) Fc.

2. The heterodimeric Fc polypeptide according to embodiment 1, wherein the natural loop comprises amino acids 325 to 331 of the Fc polypeptide, wherein the numbering of amino acids is according to the EU index.

3. The heterodimeric Fc variant according to embodiment 2, wherein the alternative amino acid sequence is a polypeptide between 7 and 15 amino acids in length.

4. The heterodimeric Fc variant according to embodiment 2, wherein the alternative amino acid sequence is a polypeptide between 8 and 15 amino acids in length.

5. The heterodimeric Fc variant according to any one of embodiments 1 to 4, wherein the target amino acid residue in FcγRIIb is Ser 135.

6. A heterodimeric Fc variant comprising a first Fc polypeptide and a second Fc polypeptide,
one of the Fc polypeptides comprising a replacement of amino acids 325 to 331 with a polypeptide between 8 and 15 amino acids in length,
wherein the heterodimeric Fc variant has increased selectivity of binding to FcγRIIb as compared to a parental Fc region,
wherein the heterodimeric Fc variant is a variant of an immunoglobulin G (IgG) Fc,
and wherein the numbering of amino acids is according to the EU index.

7. The heterodimeric Fc variant according to embodiment 6, wherein the polypeptide is derived from the sequence of a loop-forming segment of a second protein.

8. The heterodimeric Fc variant according to embodiment 7, wherein the loop-forming segment is anchored in the second protein by beta-strands.

9. The heterodimeric Fc variant according to embodiment 7 or 8, wherein in its native conformation within the second protein, the loop-forming segment has the following properties:
i) the loop-forming segment includes one or more beta-stranded amino acids at each of the N-terminus and C-terminus;
ii) the one or more beta-stranded amino acids at the C-terminus of the loop-forming segment do not form hydrogen bonds with any amino acid in the parent protein except the beta-stranded amino acids at the N-terminus of the loop-forming segment;
iii) the backbone heavy atom root mean square deviation (RMSD) of the one or more beta-stranded amino acids at the N-terminus of the loop-forming segment to one or more amino acids ending at position 324 is ≤0:85 Å, and
iv) the backbone heavy atom RMSD of the one or more beta-stranded amino acids at the C-terminus of the loop-forming segment to one or more amino acids beginning at position 332 is ≤0:85 Å.

10. The heterodimeric Fc variant according to embodiment 9, wherein the loop-forming segment further comprises the following property:
the loop-forming segment includes at least one hydrogen bond between beta-stranded amino acids at opposite termini of the loop-forming segment.

11. The heterodimeric Fc variant according to any one of embodiments 7 to 10, wherein the loop-forming segment comprises:
(a) an amino acid sequence as set forth in any one of SEQ ID NOs: 4, 5, 6, 7, 8, 9, 10, 11, 12, 13 or 14, or
(b) an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations.

12. The heterodimeric Fc variant according to embodiment 6, wherein the polypeptide comprises an amino acid sequence of Formula (I), Formula (Ia), Formula (Ib), Formula (II), Formula (III), Formula (IV), Formula (V) or Formula (VI):

Formula (I):
(I)
X¹X²WX³X⁴X⁵GX⁶X⁷T

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, F, G, H, K, L, N, Q or R.

Formula (Ia):
(Ia)
X¹X²WX³X⁴X⁵GYX⁶T

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y, and
X⁶ is A, D, E, F, G, H, K, L, N, Q or R;

Formula (Ib):
(Ib)
X¹X²WX³X⁴GGYX⁵T

wherein:
X¹ is A or S;
X² is A, D, E, F, H, I, L, N, Q, T, V or W;
X³ is D, E, F, H, N, Q, S, T or Y;
X⁴ is D, G, I or L, and
X⁵ is A, F, H, K, L or N;

Formula (II):
(II)
X¹LDX²X³GKGX⁴V

wherein:
X¹ is F or G;
X² is E, H, Q or T;
X³ is E, N, R, S or T, and
X⁴ is A, Y or V;

Formula (III):
(III)
X¹TDEX²GKGX³T

wherein:
X¹ is F or G;
X² is E or N, and
X³ is A or V;

Formula (IV):
(IV)
X¹FX²X³X⁴X⁵GEVV

wherein:
X¹ is A or D;
X² is D or N;
X³ is D, E, H, N, P, Q, S or T;
X⁴ is D, E, N, S or T, and
X⁵ is D or Q;

Formula (V):
X¹TDX²X³X⁴GEVT (V)

wherein:
X¹ is A or D;
X² is D, P or Q;
X³ is D, E or N, and
X⁴ is D or Q;

Formula (VI):
LTDX¹X²GX³PX⁴R (VI)

wherein:
X¹ is E or H;
X² is D, E or N;
X³ is R or S, and
X⁴ is I, Q or Y.

13. The heterodimeric Fc variant according to embodiment 12, wherein the polypeptide comprises an amino acid sequence of Formula (I).

14. The heterodimeric Fc variant according to embodiment 13, wherein X¹ is A or S.

15. The heterodimeric Fc variant according to embodiment 13 or 14, wherein X² is:
(i) A, D, E, F, H, I, L, N, Q, T, V or W, or
(ii) H or T.

16. The heterodimeric Fc variant according to any one of embodiments 13 to 15, wherein X³ is:
(i) A, F, H, I, S, T, V, W or Y, or
(ii) D, E, F, H, N, Q, S, T or Y, or
(iii) F, H, S, T or Y, or
(iv) E, F, H, Q, S or T, or
(v) F, H, S or T, or
(vi) E, F or S, or
(vii) F or S.

17. The heterodimeric Fc variant according to any one of embodiments 13 to 16, wherein X⁴ is:
(i) D, G, I or L, or
(ii) D or G.

18. The heterodimeric Fc variant according to any one of embodiments 13 to 17, wherein X⁵ is:
(i) A, D, E, G, H, K or R, or
(ii) G.

19. The heterodimeric Fc variant according to any one of embodiments 13 to 18, wherein X⁶ is:
(i) F, W or Y, or
(ii) Y.

20. The heterodimeric Fc variant according to any one of embodiments 13 to 19, wherein X⁷ is:
(i) A, D, E, G, H, K, L, N, Q or R, or
(ii) A, F, H, K, L or N, or
(iii) A, H, K, L or N, or
(iv) A or N.

21. The heterodimeric Fc variant according to embodiment 12, wherein the polypeptide comprises an amino acid sequence of Formula (Ia).

22. The heterodimeric Fc variant according to embodiment 21, wherein X¹ is A or S.

23. The heterodimeric Fc variant according to embodiment 21 or 22, wherein X² is:
(i) A, D, E, F, H, I, L, N, Q, T, V or W, or
(ii) H or T.

24. The heterodimeric Fc variant according to any one of embodiments 21 to 23, wherein X³ is:
(i) A, F, H, I, S, T, V, W or Y, or
(ii) D, E, F, H, N, Q, S, T or Y, or
(iii) F, H, S, T or Y, or
(iv) E, F, H, Q, S or T, or
(v) F, H, S or T, or
(vi) E, F or S, or
(vii) F or S.

25. The heterodimeric Fc variant according to any one of embodiments 21 to 24, wherein X⁴ is:
(i) D, G, I or L, or
(ii) D or G.

26. The heterodimeric Fc variant according to any one of embodiments 21 to 25, wherein X⁵ is:
(i) A, D, E, G, H, K or R, or
(ii) G.

27. The heterodimeric Fc variant according to any one of embodiments 21 to 26, wherein X⁶ is:
(i) A, D, E, G, H, K, L, N, Q or R, or
(ii) A, F, H, K, L or N, or
(iii) A, H, K, L or N, or
(iv) A or N.

28. The heterodimeric Fc variant according to embodiment 12, wherein the polypeptide comprises an amino acid sequence of Formula (Ib).

29. The heterodimeric Fc variant according to embodiment 28, wherein X² is H or T.

30. The heterodimeric Fc variant according to embodiment 28 or 29, wherein X³ is:
(i) F, H, S or Y, or
(ii) E, F, H, Q, S or T, or
(iii) F, H or S, or
(iv) E, F or S, or
(v) F or S.

31. The heterodimeric Fc variant according to any one of embodiments 28 to 30, wherein X⁴ is D or G.

32. The heterodimeric Fc variant according to any one of embodiments 28 to 31, wherein X⁵ is:
(i) A, F, H, K or L, or
(ii) A or N, or
(iii) A.

33. The heterodimeric Fc variant according to embodiment 12, wherein the polypeptide comprises an amino acid sequence of Formula (II).

34. The heterodimeric Fc variant according to embodiment 33, wherein X² is E.

35. The heterodimeric Fc variant according to embodiment 33 or 34, wherein X³ is E, N, R or S.

36. The heterodimeric Fc variant according to embodiment 33 or 34, wherein X³ is E or N.

37. The heterodimeric Fc variant according to embodiment 12, wherein the polypeptide comprises an amino acid sequence of Formula (III).

38. The heterodimeric Fc variant according to embodiment 12, wherein the polypeptide comprises an amino acid sequence of Formula (IV).

39. The heterodimeric Fc variant according to embodiment 38, wherein X¹ is D.

40. The heterodimeric Fc variant according to embodiment 38 or 39, wherein X² is D.

41. The heterodimeric Fc variant according to any one of embodiments 38 to 40, wherein X³ is E, H, N, S or T.

42. The heterodimeric Fc variant according to any one of embodiments 38 to 41, wherein X⁴ is D, N, S or T.

43. The heterodimeric Fc variant according to embodiment 12, wherein the polypeptide comprises an amino acid sequence of Formula (V).

44. The heterodimeric Fc variant according to embodiment 12, wherein the polypeptide comprises an amino acid sequence of Formula (VI).

45. The heterodimeric Fc variant according to embodiment 44, wherein X¹ is E.

46. The heterodimeric Fc variant according to embodiment 44 or 45, wherein X⁴ is I or Y.

47. The heterodimeric Fc variant according to embodiment 12, wherein the polypeptide comprises an amino acid sequence as set forth in any one of SEQ ID NOs: 4-172.

48. The heterodimeric Fc variant according to embodiment 12, wherein the polypeptide comprises an amino acid sequence as set forth in any one of SEQ ID NOs: 4-90.

49. The heterodimeric Fc variant according to embodiment 12, wherein the polypeptide comprises:
(a) an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90, or
(b) an amino acid sequence as set forth in any one of SEQ ID NOs: 6, 8, 47, 68 or 73.

50. The heterodimeric Fc variant according to any one of embodiments 6 to 49, further comprising one or more additional amino acid mutations in the CH2 domain of the heterodimeric Fc variant.

51. The heterodimeric Fc variant according to embodiment 50, wherein the one or more additional amino acid mutations comprise a mutation at position 236.

52. The heterodimeric Fc variant according to embodiment 51, wherein both the first Fc polypeptide and the second Fc polypeptide comprise a mutation at position 236.

53. The heterodimeric Fc variant according to embodiment 52, wherein the mutation at position 236 in the first and second Fc polypeptides is symmetric.

54. The heterodimeric Fc variant according to embodiment 53, wherein the mutation at position 236 is selected from G236D, G236N and G236K.

55. The heterodimeric Fc variant according to embodiment 53, wherein the mutation at position 236 is G236D or G236N.

56. The heterodimeric Fc variant according to embodiment 51 or 52, wherein the mutation at position 236 in the first and second Fc polypeptides is asymmetric.

57. The heterodimeric Fc variant according to embodiment 56, wherein the replacement of amino acids 325 to 331 is in the second Fc polypeptide, and wherein the first Fc polypeptide comprises a mutation at position 236 selected from G236A, G236D, G236E, G236F, G236H, G236I, G236L, G236N, G236P, G236Q, G236S, G236T, G236V, G236W and G236Y, and the second Fc polypeptide comprises a mutation at position 236 selected from G236D, G236E, G236K, G236N and G236T.

58. The heterodimeric Fc variant according to embodiment 56, wherein the replacement of amino acids 325 to 331 is in the second Fc polypeptide, and wherein the first Fc polypeptide comprises a mutation at position 236 selected from G236A, G236D, G236E, G236F, G236H, G236I, G236L, G236N, G236P, G236Q, G236S, G236T, G236V, G236W and G236Y, and the second Fc polypeptide comprises the mutation G236D or does not comprise a mutation at position 236.

59. The heterodimeric Fc variant according to embodiment 56, wherein the replacement of amino acids 325 to 331 is in the second Fc polypeptide, and wherein the first Fc polypeptide comprises the mutation G236N or does not comprise a mutation at position 236, and the second Fc polypeptide comprises a mutation at position 236 selected from G236D, G236E, G236K, G236N and G236T.

60. The heterodimeric Fc variant according to embodiment 56, wherein the replacement of amino acids 325 to 331 is in the second Fc polypeptide, and wherein the first Fc polypeptide comprises a mutation at position 236 selected from G236D, G236K and G236N, and the second Fc polypeptide comprises a mutation at position 236 selected from G236D and G236N or does not comprise a mutation at position 236.

61. The heterodimeric Fc variant according to embodiment 56, wherein the replacement of amino acids 325 to 331 is in the second Fc polypeptide, and wherein the first Fc polypeptide comprises the mutation G236N and the second Fc polypeptide comprises the mutation G236D.

62. The heterodimeric Fc variant according to any one of embodiments 6 to 61, wherein the replacement of amino acids 325 to 331 is in the second Fc polypeptide, and the second Fc polypeptide further comprises one or mutations selected from S239D, S239E, V266I, V266L, S267A, S267I, S267V, S267Q and H268D.

63. The heterodimeric Fc variant according to any one of embodiments 6 to 61, wherein the replacement of amino acids 325 to 331 is in the second Fc polypeptide, and the second Fc polypeptide further comprises one or mutations selected from S239D, S239E, V266L, S267A, S267I, S267V and H268D.

64. The heterodimeric Fc variant according to embodiment 63, wherein the second Fc polypeptide comprises: (i) the mutation S239D or S239E, and/or (ii) the mutation H268D, and/or (iii) the mutation S267A, S267I or S267V.

65. The heterodimeric Fc variant according to embodiment 63, wherein the second Fc polypeptide comprises the mutations S239D, H268D and S267V.

66. The heterodimeric Fc variant according to any one of embodiments 6 to 65, wherein the replacement of amino acids 325 to 331 is in the second Fc polypeptide, and wherein the first Fc polypeptide further comprises a mutation at one or more of positions 234, 235, 237 and 239.

67. The heterodimeric Fc variant according to embodiment 66, wherein:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

68. The heterodimeric Fc variant according to embodiment 66, wherein:
(i) the mutation at position 234 is selected from L234D, L234F, L234Q, L234T and L234W,
(ii) the mutation at position 235 is selected from L235A, L235D, L235E, L235F, L235H, L235R, L235W and L235Y,
(iii) the mutation at position 237 is selected from G237A, G237D, G237L and G237N, and
(iv) the mutation at position 239 is selected from S239A, S239G, S239H, S239T and S239Y.

69. The heterodimeric Fc variant according to embodiment 66, wherein the first Fc polypeptide comprises the mutations L234D and/or L235F.

70. The heterodimeric Fc variant according to any one of embodiments 6 to 69, wherein the replacement of amino acids 325 to 331 is in the second Fc polypeptide, and wherein the second Fc polypeptide further comprises a mutation at one or more of positions 234, 235, 237, 240, 263, 264, 266, 269, 271, 273, 323 and 332.

71. The heterodimeric Fc variant according to embodiment 70, wherein:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

72. The heterodimeric Fc variant according to embodiment 70 or 71, wherein the second Fc polypeptide comprises a mutation at one or more of positions 271, 323 and 332.

73. The heterodimeric Fc variant according to embodiment 72, wherein:
(i) the mutation at position 271 is P271D,
(ii) the mutation at position 323 is V323A, and
(iii) the mutation at position 332 is selected from I332F and I332L.

74. The heterodimeric Fc variant according to any one of embodiments 6 to 73, wherein the first Fc polypeptide and second Fc polypeptide further comprise one or more mutations selected from: A287F, T250V, L309Q and M428F.

75. The heterodimeric Fc variant according to embodiment 74, wherein the first Fc polypeptide and second Fc polypeptide further comprise the mutations A287F/M428F, A287F/T250V, M428F/T250V or T250V/L309Q.

76. The heterodimeric Fc variant according to embodiment 6, wherein the heterodimeric Fc variant comprises the amino acid mutations as set out for any one of the variants shown in Table 6.22, 6.24, 6.25 or 6.27.

77. The heterodimeric Fc variant according to embodiment 6, wherein:
(i) the first Fc polypeptide comprises the mutations G236N_G237D, and the second Fc polypeptide comprises the mutations Template 1 (D329*I)+G236D_G237F_S239D_S267V_H268D (Variant 31186);
(ii) the first Fc polypeptide comprises the mutations L235F_G236N_G237A, and the second Fc polypeptide comprises the mutations Template 1 (D329*I)+G236D_G237F_S239D_S267V_H268D (Variant 31187);
(iii) the first Fc polypeptide comprises the mutations L235F_G236N_G237A, and the second Fc polypeptide comprises the mutations Template 1 (G330*K)+G236D_G237F_S239D_S267V_H268D (Variant 31188);
(iv) the first Fc polypeptide comprises the mutations G236N_G237D, and the second Fc polypeptide comprises the mutations Template 7 (E328*H_E329*R_A331*BY)+G236D_G237F_S239D_S267V_H268D (Variant 31191);
(v) the first Fc polypeptide comprises the mutations L235F_G236N_G237A, and the second Fc polypeptide comprises the mutations Template 1 (D329*I)+G236D_G237F_S239D_S267V_H268D_I332L (Variant 31213);
(vi) the first Fc polypeptide comprises the mutations L235F_G236N_G237A_T250V_A287F, and the second Fc polypeptide comprises the mutations Template 1 (D329*I)+G236D_G237F_S239D_T250V_S267V_H268D_A287F (Variant 31274);
(vii) the first Fc polypeptide comprises the mutations L235F_G236N_G237A_T250V_M428F, and the second Fc polypeptide comprises the mutations Template 1 (D329*I)+G236D_G237F_S239D_T250V_S267V_H268D_M428F (Variant 31275);
(viii) the first Fc polypeptide comprises the mutations L235F_G236N_G237A_A287F_M428F, and the second Fc polypeptide comprises the mutations Template 1 (D329*I)+G236D_G237F_S239D_S267V_H268D_A287F_M428F (Variant 31276);
(ix) the first Fc polypeptide comprises the mutations G236N_G237D, and the second Fc polypeptide comprises the mutations Template 1 (D329*I)+G236D_G237F_S239D_S267V_H268D_I332L (Variant 32210);
(x) the first Fc polypeptide comprises the mutations G236N_G237E, and the second Fc polypeptide comprises the mutations Template 1 (D329*I)+G236D_G237F_S239D_S267V_H268D_I332L (Variant 32211);
(xi) the first Fc polypeptide comprises the mutation G236N, and the second Fc polypeptide comprises the mutations Template 1 (D329*I)+G236D_G237F_S239D_S267V_H268D_I332L (Variant 32212);
(xii) the first Fc polypeptide comprises the mutations L235D_G236N_G237A, and the second Fc polypeptide comprises the mutations Template 1 (D329*I)+G236D_G237F_S239D_S267V_H268D_I332L (Variant 32226);
(xiii) the first Fc polypeptide comprises the mutations L235E_G236N_G237A, and the second Fc polypeptide comprises the mutations Template 1 (D329*I)+G236D_G237F_S239D_S267V_H268D_I332L (Variant 32227);
(xiv) the first Fc polypeptide comprises the mutations L235V_G236N_G237A, and the second Fc polypeptide comprises the mutations Template 1 (D329*I)+G236D_G237F_S239D_S267V_H268D_I332L (Variant 32230);
(xv) the first Fc polypeptide comprises the mutations L235Y_G236N_G237A, and the second Fc polypeptide comprises the mutations Template 1 (D329*I)+G236D_G237F_S239D_S267V_H268D_I332L (Variant 32231);
(xvi) the first Fc polypeptide comprises the mutations G236N_G237A_S239P, and the second Fc polypeptide comprises the mutations Template 1 (D329*I)+G236D_G237F_S239D_S267V_H268D_I332L (Variant 32242);
(xvii) the first Fc polypeptide comprises the mutations L234D_G236N_G237A, and the second Fc polypeptide comprises the mutations Template 7+G236D_G237F_S239D_S267V_H268D (Variant 32282);
(xviii) the first Fc polypeptide comprises the mutations L235D_G236N_G237A, and the second Fc polypeptide comprises the mutations Template 7+G236D_G237F_S239D_S267V_H268D (Variant 32284);
(xix) the first Fc polypeptide comprises the mutations G236N_G237A_S239G, and the second Fc polypeptide comprises the mutations Template 7+G236D_G237F_S239D_S267V_H268D (Variant 32287);
(xx) the first Fc polypeptide comprises the mutations G236N_G237A_S239H, and the second Fc polypeptide comprises the mutations Template 7+G236D_G237F_S239D_S267V_H268D (Variant 32288);
(xxi) the first Fc polypeptide comprises the mutations G236N_G237E, and the second Fc polypeptide comprises the mutations Template 7+G236D_G237F_S239D_S267V_H268D (Variant 32296);
(xxii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(xxiii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(xxiv) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(xxv) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294), or
(xxvi) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

78. The heterodimeric Fc variant according to any one of embodiments 1 to 77, wherein the heterodimeric Fc variant is a variant of an IgG1 Fc.

79. The heterodimeric Fc variant according to embodiment 78, wherein the heterodimeric Fc variant is a variant of a human IgG1 Fc.

80. The heterodimeric Fc variant according to any one of embodiments 1 to 79, wherein the selectivity of binding to FcγRIIb of the heterodimeric Fc variant is increased by at least 1.5-fold or by at least 2-fold over the parental Fc region, and wherein:

Fold Difference in FcγRIIb Selectivity=Fold Difference in FcγRIIb Affinity/Fold Difference in FcγRIIaR Affinity,

wherein:

Fold Difference in FcγRIIb Affinity=K_{D} FcγRIIb (parental)/K_{D} FcγRIIb (variant),

and

Fold Difference in FcγRIIaR Affinity=K_{D} FcγRIIaR (parental)/K_{D} FcγRIIaR (variant).

81. The heterodimeric Fc variant according to any one of embodiments 1 to 80, wherein the heterodimeric Fc variant has increased binding affinity for FcγRIIb as compared to the parental Fc region.

82. The heterodimeric Fc variant according to embodiment 81, wherein the binding affinity of the heterodimeric Fc variant for FcγRIIb is increased by at least 10-fold over the parental Fc region, and wherein:

Fold Difference in FcγRIIb Affinity=K_{D} FcγRIIb (parental)/K_{D} FcγRIIb (variant).

83. A polypeptide comprising the heterodimeric Fc variant according to any one of embodiments 1 to 82 and one or more proteinaceous moieties fused or covalently attached to the heterodimeric Fc variant.

84. The polypeptide according to embodiment 83, wherein the polypeptide is an antibody and the one or more proteinaceous moieties are one or more antigen-binding domains.

85. The polypeptide according to embodiment 84, wherein at least one of the antigen-binding domains binds to a tumour-associated antigen or tumour-specific antigen.

86. A pharmaceutical composition comprising the heterodimeric Fc variant according to any one of embodiments 1 to 82, or the polypeptide according to any one of embodiments 83 to 85, and a pharmaceutically acceptable carrier or diluent.

87. A polypeptide according to any one of embodiments 83 to 85 for use in therapy.

88. A polypeptide according to embodiment 85 for use in the treatment of cancer.

89. Nucleic acid encoding the heterodimeric Fc variant according to any one of embodiments 1 to 82, or the polypeptide according to any one of embodiments 83 to 85.

90. A host cell comprising the nucleic acid according to embodiment 89.

91. A method of preparing the heterodimeric Fc variant according to any one of embodiments 1 to 82, or the polypeptide according to any one of embodiments 83 to 85, comprising expressing nucleic acid encoding the heterodimeric Fc variant or the polypeptide in a host cell.

92. A method of preparing a heterodimeric Fc variant having increased selectivity for a target receptor as compared to a parental Fc region, the heterodimeric Fc variant comprising a first Fc polypeptide and a second Fc polypeptide, the method comprising:
(a) using an in silico model of the parental Fc region complexed with the target receptor:
   (i) inserting a sequence of one or more amino acid residues into a natural loop of one of the Fc polypeptides such that the natural loop is extended in length to provide a candidate variant,
   (ii) determining the distance of at least one of the amino acid residues of the inserted sequence from a target amino acid residue in the receptor, and
   (iii) selecting the candidate variant as the heterodimeric Fc variant if the at least one amino acid residue of the inserted sequence is within a heavy atom to heavy atom distance of 3 Å of the target amino acid residue in the receptor,
(b) preparing nucleic acid encoding the heterodimeric Fc variant,
(c) expressing the nucleic acid in a host cell to provide the heterodimeric Fc variant,
   wherein the target receptor is FcγRIIb.

93. A heterodimeric Fc variant comprising a first Fc polypeptide and a second Fc polypeptide, the heterodimeric Fc variant having increased selectivity of binding to FcγRIIb as compared to a parental Fc region, the heterodimeric Fc variant comprising an asymmetric mutation at position 236,
wherein one of the Fc polypeptides comprises the mutation G236N or G236D,
wherein the heterodimeric Fc variant is a variant of an immunoglobulin G (IgG) Fc,
and wherein the numbering of amino acids is according to the EU index.

94. The heterodimeric Fc variant according to embodiment 93, wherein the first Fc polypeptide comprises the mutation G236N or G236D, and the second Fc polypeptide does not comprise a mutation at position 236.

95. The heterodimeric Fc variant according to embodiment 93, wherein the first Fc polypeptide comprises the mutation G236N or G236D, and the second Fc polypeptide comprises a different mutation at position 236.

96. The heterodimeric Fc variant according to embodiment 95, wherein the first Fc polypeptide comprises the mutation G236N, and the second Fc polypeptide comprises the mutation G236D, G236K or G236S.

97. The heterodimeric Fc variant according to embodiment 95, wherein the first Fc polypeptide comprises the mutation G236N, and the second Fc polypeptide comprises the mutation G236D.

98. The heterodimeric Fc variant according to embodiment 95, wherein the first Fc polypeptide comprises the mutation G236D, and the second Fc polypeptide comprises the mutation G236N, G236Q, G236K, G236E or G236H.

99. The heterodimeric Fc variant according to any one of embodiments 93 to 98, wherein the first Fc polypeptide and/or the second Fc polypeptide further comprises one or more additional amino acid mutations in the CH2 domain of the heterodimeric Fc variant.

100. The heterodimeric Fc variant according to embodiment 99, wherein the second Fc polypeptide further comprises one or mutations selected from S239D, S239E, V266I, V266L, S267A, S267I, S267V, S267Q and H268D.

101. The heterodimeric Fc variant according to embodiment 99, wherein the second Fc polypeptide further comprises one or mutations selected from S239D, S239E, V266L, S267A, S267I, S267Q, S267V and H268D.

102. The heterodimeric Fc variant according to embodiment 99, wherein the second Fc polypeptide further comprises:
a) the mutation S239D or S239E; or
b) the mutation H268D, or
c) the mutation S239D or S239E, and the mutation H268D.

103. The heterodimeric Fc variant according to embodiment 99, wherein the second Fc polypeptide further comprises the mutations S239D and H268D.

104. The heterodimeric Fc variant according to any one of embodiments 93 to 103, wherein the heterodimeric Fc variant is a Strategy 1/3 variant.

105. The heterodimeric Fc variant according to any one of embodiments 93 to 104, wherein the second Fc polypeptide further comprises the mutation S267A, S267I or S267V.

106. The heterodimeric Fc variant according to any one of embodiments 93 to 105, wherein amino acids 325 to 331 in the second Fc polypeptide are replaced with a polypeptide between 8 and 15 amino acids in length.

107. The heterodimeric Fc variant according to embodiment 106, wherein the polypeptide is derived from a loop-forming segment of a second protein.

108. The heterodimeric Fc variant according to embodiment 107, wherein the loop-forming segment is anchored in the second protein by beta-strands.

109. The heterodimeric Fc variant according to embodiment 107 or 108, wherein in its native conformation within the second protein, the loop-forming segment has the following properties:
i) the loop-forming segment includes one or more beta-stranded amino acids at each of the loop N-terminus and C-terminus;
ii) the one or more beta-stranded amino acids at the C-terminus of the loop-forming segment do not form hydrogen bonds with any amino acid in the parent protein except the beta-stranded amino acids at the N-terminus of the loop-forming segment;
iii) the backbone heavy atom root mean square deviation (RMSD) of the one or more beta-stranded amino acids at the N-terminus of the loop-forming segment to one or more amino acids ending at position 324 is ≤0:85 Å, and
iv) the backbone heavy atom RMSD of the one or more beta-stranded amino acids at the C-terminus of the loop-forming segment to one or more amino acids beginning at position 332 is ≤0:85 Å.

110. The heterodimeric Fc variant according to embodiment 109, wherein the loop-forming segment further comprises the following property:
the loop-forming segment includes at least one hydrogen bond between beta-stranded amino acids at opposite termini of the loop-forming segment.

111. The heterodimeric Fc variant according to any one of embodiments 106 to 110, wherein the polypeptide comprises:
(a) an amino acid sequence as set forth in any one of SEQ ID NOs: 4, 5, 6, 7, 8, 9, 10, 11, 12, 13 or 14, or
(b) an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations.

112. The heterodimeric Fc variant according to embodiment 106, wherein the polypeptide comprises an amino acid sequence of Formula (I), Formula (Ia), Formula (Ib), Formula (II), Formula (III), Formula (IV), Formula (V) or Formula (VI):

Formula (I):
X¹X²WX³X⁴X⁵GX⁶X⁷T (I)

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, F, G, H, K, L, N, Q or R;

Formula (Ia):
X¹X²WX³X⁴X⁵GYX⁶T (Ia)

wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y, and
X⁶ is A, D, E, F, G, H, K, L, N, Q or R;

Formula (Ib):
X¹X²WX³X⁴GGYX⁵T (Ib)

wherein:
X¹ is A or S;
X² is A, D, E, F, H, I, L, N, Q, T, V or W;
X³ is D, E, F, H, N, Q, S, T or Y;
X⁴ is D, G, I or L, and
X⁵ is A, F, H, K, L or N;

Formula (II):
X¹LDX²X³GKGX⁴V (II)

wherein:
X¹ is F or G;
X² is E, H, Q or T;
X³ is E, N, R, S or T, and
X⁴ is A, Y or V;

Formula (III):
X¹TDEX²GKGX³T (III)

wherein:
X¹ is F or G;
X² is E or N, and
X³ is A or V;

Formula (IV):
X¹FX²X³X⁴X⁵GEVV (IV)

wherein:
X¹ is A or D;
X² is D or N;
X³ is D, E, H, N, P, Q, S or T;
X⁴ is D, E, N, S or T, and
X⁵ is D or Q;

Formula (V):
X¹TDX²X³X⁴GEVT (V)

wherein:
X¹ is A or D;
X² is D, P or Q;
X³ is D, E or N, and
X⁴ is D or Q;

Formula (VI):
LTDX¹X²GX³PX⁴R (VI)

wherein:
X¹ is E or H;
X² is D, E or N;
X³ is R or S, and
X⁴ is I, Q or Y.

113. The heterodimeric Fc variant according to embodiment 112, wherein the polypeptide comprises an amino acid sequence of Formula (I).

114. The heterodimeric Fc variant according to embodiment 113, wherein X¹ is A or S.

115. The heterodimeric Fc variant according to embodiment 113 or 114, wherein X² is:
(i) A, D, E, F, H, I, L, N, Q, T, V or W, or
(ii) H or T.

116. The heterodimeric Fc variant according to any one of embodiments 113 to 115, wherein X³ is:
(i) A, F, H, I, S, T, V, W or Y, or
(ii) D, E, F, H, N, Q, S, T or Y, or
(iii) F, H, S, T or Y, or
(iv) E, F, H, Q, S or T, or
(v) F, H, S or T, or
(vi) E, F or S, or
(vii) F or S.

117. The heterodimeric Fc variant according to any one of embodiments 113 to 116, wherein X⁴ is:
(i) D, G, I or L, or
(ii) D or G.

118. The heterodimeric Fc variant according to any one of embodiments 113 to 117, wherein X⁵ is:
(i) A, D, E, G, H, K or R, or
(ii) G.

119. The heterodimeric Fc variant according to any one of embodiments 113 to 118, wherein X⁶ is:
(i) F, W or Y, or
(ii) Y.

120. The heterodimeric Fc variant according to any one of embodiments 113 to 119, wherein X⁷ is:
(i) A, D, E, G, H, K, L, N, Q or R, or
(ii) A, F, H, K, L or N, or
(iii) A, H, K, L or N, or
(iv) A or N.

121. The heterodimeric Fc variant according to embodiment 112, wherein the polypeptide comprises an amino acid sequence of Formula (Ia).

122. The heterodimeric Fc variant according to embodiment 121, wherein X¹ is A or S.

123. The heterodimeric Fc variant according to embodiment 121 or 122, wherein X² is:
(i) A, D, E, F, H, I, L, N, Q, T, V or W, or
(ii) H or T.

124. The heterodimeric Fc variant according to any one of embodiments 121 to 123, wherein X³ is:
(i) A, F, H, I, S, T, V, W or Y, or
(ii) D, E, F, H, N, Q, S, T or Y, or
(iii) F, H, S, T or Y, or
(iv) E, F, H, Q, S or T, or
(v) F, H, S or T, or
(vi) E, F or S, or
(vii) F or S.

125. The heterodimeric Fc variant according to any one of embodiments 121 to 124, wherein X⁴ is:
(i) D, G, I or L, or
(ii) D or G.

126. The heterodimeric Fc variant according to any one of embodiments 121 to 125, wherein X⁵ is:
(i) A, D, E, G, H, K or R, or
(ii) G.

127. The heterodimeric Fc variant according to any one of embodiments 121 to 126, wherein X⁶ is:
(i) A, D, E, G, H, K, L, N, Q or R, or
(ii) A, F, H, K, L or N, or
(iii) A, H, K, L or N, or
(iv) A or N.

128. The heterodimeric Fc variant according to embodiment 112, wherein the polypeptide comprises an amino acid sequence of Formula (Ib).

129. The heterodimeric Fc variant according to embodiment 126, wherein X² is H or T.

130. The heterodimeric Fc variant according to embodiment 128 or 129, wherein X³ is:
(i) F, H, S or Y, or
(ii) E, F, H, Q, S or T, or
(iii) F, H or S, or
(iv) E, F or S, or
(v) F or S.

131. The heterodimeric Fc variant according to any one of embodiments 128 to 130, wherein X⁴ is D or G.

132. The heterodimeric Fc variant according to any one of embodiments 128 to 131, wherein X⁵ is:
(i) A, F, H, K or L, or
(ii) A or N, or
(iii) A.

133. The heterodimeric Fc variant according to embodiment 112, wherein the polypeptide comprises an amino acid sequence of Formula (II).

134. The heterodimeric Fc variant according to embodiment 133, wherein X² is E.

135. The heterodimeric Fc variant according to embodiment 133 or 134, wherein X³ is E, N, R or S.

136. The heterodimeric Fc variant according to embodiment 133 or 134, wherein X³ is E or N.

137. The heterodimeric Fc variant according to embodiment 112, wherein the polypeptide comprises an amino acid sequence of Formula (III).

138. The heterodimeric Fc variant according to embodiment 112, wherein the polypeptide comprises an amino acid sequence of Formula (IV).

139. The heterodimeric Fc variant according to embodiment 138, wherein X¹ is D.

140. The heterodimeric Fc variant according to embodiment 138 or 139, wherein X² is D.

141. The heterodimeric Fc variant according to any one of embodiments 138 to 140, wherein X³ is E, H, N, S or T.

142. The heterodimeric Fc variant according to any one of embodiments 138 to 141, wherein X⁴ is D, N, S or T.

143. The heterodimeric Fc variant according to embodiment 112, wherein the polypeptide comprises an amino acid sequence of Formula (V).

144. The heterodimeric Fc variant according to embodiment 112, wherein the polypeptide comprises an amino acid sequence of Formula (VI).

145. The heterodimeric Fc variant according to embodiment 144, wherein X¹ is E.

146. The heterodimeric Fc variant according to embodiment 144 or 145, wherein X⁴ is I or Y.

147. The heterodimeric Fc variant according to embodiment 106, wherein the polypeptide comprises an amino acid sequence as set forth in any one of SEQ ID NOs: 4-172.

148. The heterodimeric Fc variant according to embodiment 106, wherein the polypeptide comprises an amino acid sequence as set forth in any one of SEQ ID NOs: 4-90.

149. The heterodimeric Fc variant according to embodiment 106, wherein the polypeptide comprises an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90.

150. The heterodimeric Fc variant according to any one of embodiments 93 to 149, wherein the second Fc polypeptide further comprises the mutation S267V.

151. The heterodimeric Fc variant according to any one of embodiments 93 to 150, wherein the first Fc polypeptide and/or the second Fc polypeptide further comprises a mutation at position 237.

152. The heterodimeric Fc variant according to embodiment 151, wherein the first Fc polypeptide or the second Fc polypeptide comprises the mutation G236N and the same Fc polypeptide further comprises a mutation at position 237 selected from G237A, G237D, G237F, G237H, G237L, G237N, G237P, G237S, G237V, G237W and G237Y.

153. The heterodimeric Fc variant according to embodiment 151, wherein the first Fc polypeptide or the second Fc polypeptide comprises the mutation G236N and the same Fc polypeptide further comprises the mutation G237A.

154. The heterodimeric Fc variant according to embodiment 151, wherein the first Fc polypeptide or the second Fc polypeptide comprises the mutation G236D and the same Fc polypeptide further comprises a mutation at position 237 selected from G237F, G237I, G237K, G237L, G237Q, G237T, G237V and G237Y.

154. The heterodimeric Fc variant according to embodiment 151, wherein the first Fc polypeptide or the second Fc polypeptide comprises the mutation G236D and the same Fc polypeptide further comprises the mutation G237F.

155. The heterodimeric Fc variant according to any one of embodiments 93 to 154, wherein the first Fc polypeptide comprises the mutation G236N, and wherein the first Fc polypeptide further comprises a mutation at one or more of positions 234, 235, 237 and 239.

156. The heterodimeric Fc variant according to embodiment 155, wherein:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

157. The heterodimeric Fc variant according to embodiment 155, wherein:
(i) the mutation at position 234 is selected from L234D, L234F, L234Q, L234T and L234W,
(ii) the mutation at position 235 is selected from L235A, L235D, L235E, L235F, L235H, L235R, L235W and L235Y,
(iii) the mutation at position 237 is selected from G237A, G237D, G237L and G237N, and
(iv) the mutation at position 239 is selected from S239A, S239G, S239H, S239T and S239Y.

158. The heterodimeric Fc variant according to embodiment 155, wherein the first Fc polypeptide further comprises the mutation L234D.

159. The heterodimeric Fc variant according to embodiment 155 or 158, wherein the first Fc polypeptide further comprises the mutation L235F.

160. The heterodimeric Fc variant according to any one of embodiments 93 to 159, wherein the second Fc polypeptide comprises the mutation G236D, and wherein the second Fc polypeptide further comprises a mutation at one or more of positions 234, 235, 237, 240, 263, 264, 266, 269, 271, 273, 323 and 332.

161. The heterodimeric Fc variant according to embodiment 160, wherein:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

162. The heterodimeric Fc variant according to embodiment 160, wherein:
(i) the mutation at position 271 is P271D,
(ii) the mutation at position 323 is V323A, and
(iii) the mutation at position 332 is selected from I332F and I332L.

163. The heterodimeric Fc variant according to embodiment 93, wherein the heterodimeric Fc variant comprises the amino acid mutations as set out for any one of the variants shown in Table 6.22, 6.24, 6.25 or 6.27.

164. The heterodimeric Fc variant according to embodiment 93, wherein:
(i) the first Fc polypeptide comprises the mutations G236N_G237D, and the second Fc polypeptide comprises the mutations Template 1 (D329*I)+G236D_G237F_S239D_S267V_H268D (Variant 31186);
(ii) the first Fc polypeptide comprises the mutations L235F_G236N_G237A, and the second Fc polypeptide comprises the mutations Template 1 (D329*I)+G236D_G237F_S239D_S267V_H268D (Variant 31187);
(iii) the first Fc polypeptide comprises the mutations L235F_G236N_G237A, and the second Fc polypeptide comprises the mutations Template 1 (G330*K)+G236D_G237F_S239D_S267V_H268D (Variant 31188);
(iv) the first Fc polypeptide comprises the mutations G236N_G237D, and the second Fc polypeptide comprises the mutations Template 7 (E328*H_E329*R_A331*BY)+G236D_G237F_S239D_S267V_H268D (Variant 31191);
(v) the first Fc polypeptide comprises the mutations L235F_G236N_G237A, and the second Fc polypeptide comprises the mutations Template 1 (D329*I)+G236D_G237F_S239D_S267V_H268D_I332L (Variant 31213);
(vi) the first Fc polypeptide comprises the mutations L235F_G236N_G237A_T250V_A287F, and the second Fc polypeptide comprises the mutations Template 1 (D329*I)+G236D_G237F_S239D_T250V_S267V_H268D_A287F (Variant 31274);
(vii) the first Fc polypeptide comprises the mutations L235F_G236N_G237A_T250V_M428F, and the second Fc polypeptide comprises the mutations Template 1 (D329*I)+G236D_G237F_S239D_T250V_S267V_H268D_M428F (Variant 31275);
(viii) the first Fc polypeptide comprises the mutations L235F_G236N_G237A_A287F_M428F, and the second Fc polypeptide comprises the mutations Template 1 (D329*I)+G236D_G237F_S239D_S267V_H268D_A287F_M428F (Variant 31276);
(ix) the first Fc polypeptide comprises the mutations G236N_G237D, and the second Fc polypeptide comprises the mutations Template 1 (D329*I)+G236D_G237F_S239D_S267V_H268D_I332L (Variant 32210);
(x) the first Fc polypeptide comprises the mutations G236N_G237E, and the second Fc polypeptide comprises the mutations Template 1 (D329*I)+G236D_G237F_S239D_S267V_H268D_I332L (Variant 32211);
(xi) the first Fc polypeptide comprises the mutation G236N, and the second Fc polypeptide comprises the mutations Template 1 (D329*I)+G236D_G237F_S239D_S267V_H268D_I332L (Variant 32212);
(xii) the first Fc polypeptide comprises the mutations L235D_G236N_G237A, and the second Fc polypeptide comprises the mutations Template 1 (D329*I)+G236D_G237F_S239D_S267V_H268D_I332L (Variant 32226);
(xiii) the first Fc polypeptide comprises the mutations L235E_G236N_G237A, and the second Fc polypeptide comprises the mutations Template 1 (D329*I)+G236D_G237F_S239D_S267V_H268D_I332L (Variant 32227);
(xiv) the first Fc polypeptide comprises the mutations L235V_G236N_G237A, and the second Fc polypeptide comprises the mutations Template 1 (D329*I)+G236D_G237F_S239D_S267V_H268D_I332L (Variant 32230);
(xv) the first Fc polypeptide comprises the mutations L235Y_G236N_G237A, and the second Fc polypeptide comprises the mutations Template 1 (D329*I)+G236D_G237F_S239D_S267V_H268D_I332L (Variant 32231);
(xvi) the first Fc polypeptide comprises the mutations G236N_G237A_S239P, and the second Fc polypeptide comprises the mutations Template 1 (D329*I)+G236D_G237F_S239D_S267V_H268D_I332L (Variant 32242);
(xvii) the first Fc polypeptide comprises the mutations L234D_G236N_G237A, and the second Fc polypeptide comprises the mutations Template 7+G236D_G237F_S239D_S267V_H268D (Variant 32282);
(xviii) the first Fc polypeptide comprises the mutations L235D_G236N_G237A, and the second Fc polypeptide comprises the mutations Template 7+G236D_G237F_S239D_S267V_H268D (Variant 32284);
(xix) the first Fc polypeptide comprises the mutations G236N_G237A_S239G, and the second Fc polypeptide comprises the mutations Template 7+G236D_G237F_S239D_S267V_H268D (Variant 32287);
(xx) the first Fc polypeptide comprises the mutations G236N_G237A_S239H, and the second Fc polypeptide comprises the mutations Template 7+G236D_G237F_S239D_S267V_H268D (Variant 32288);
(xxi) the first Fc polypeptide comprises the mutations G236N_G237E, and the second Fc polypeptide comprises the mutations Template 7+G236D_G237F_S239D_S267V_H268D (Variant 32296);
(xxii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(xxiii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(xxiv) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(xxv) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294), or
(xxvi) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

165. The heterodimeric Fc variant according to any one of embodiments 93 to 103, wherein the heterodimeric Fc variant is a Strategy 2 variant.

166. The heterodimeric Fc variant according to any one of embodiments 93 to 103 and 165, wherein the first Fc polypeptide further comprises a mutation at one or more positions selected from 234, 268, 327, 330 and 331.

167. The heterodimeric Fc variant according to embodiment 166, wherein:
(i) the mutation at position 234 is selected from L234A, L234F, L234G, L234H, L234I, L234N, L234P, L234Q, L234S, L234T, L234V, L234W and L234Y,
(ii) the mutation at position 268 is selected from H268A, H268D, H268E, H268F, H268G, H268I, H268K, H268L, H268N, H268P, H268Q, H268R, H268S, H268T, H268V, H268W and H268Y,
(iii) the mutation at position 327 is selected from A327E and A327G;
(iv) the mutation at position 330 is selected from A330K, A330H, A330Q, A330R, A330S and A330T, and
(v) the mutation at position 331 is selected from P331A, P331D, P331E, P331H, P331Q and P331S.

168. The heterodimeric Fc variant according to embodiment 166 or 167, wherein the first Fc polypeptide further comprises a mutation at position 234 selected from L234A, L234F, L234G, L234H, L234I, L234N, L234P, L234Q, L234S, L234T, L234V, L234W and L234Y.

169. The heterodimeric Fc variant according to embodiment 168, wherein the mutation at position 234 is L234F.

170. The heterodimeric Fc variant according to any one of embodiments 166 to 169, wherein the first Fc polypeptide further comprises a mutation at position 268 selected from H268A, H268D, H268E, H268F, H268G, H268I, H268K, H268L, H268N, H268P, H268Q, H268R, H268S, H268T, H268V, H268W and H268Y.

171. The heterodimeric Fc variant according to embodiment 170, wherein the mutation at position 268 is H268Q.

172. The heterodimeric Fc variant according to any one of embodiments 166 to 171, wherein the first Fc polypeptide further comprises a mutation at position 327 selected from A327E and A327G.

173. The heterodimeric Fc variant according to embodiment 172, wherein the mutation at position 327 is A327G.

174. The heterodimeric Fc variant according to any one of embodiments 166 to 173, wherein the first Fc polypeptide further comprises a mutation at position 330 selected from A330K, A330H, A330Q, A330R, A330S and A330T.

175. The heterodimeric Fc variant according to embodiment 174, wherein the mutation at position 330 is A330K or A330T.

176. The heterodimeric Fc variant according to embodiment 174, wherein the mutation at position 330 is A330K.

177. The heterodimeric Fc variant according to any one of embodiments 166 to 176, wherein the first Fc polypeptide further comprises a mutation at position 331 selected from P331A, P331D, P331E, P331H, P331Q and P331S.

178. The heterodimeric Fc variant according to embodiment 177, wherein the mutation at position 331 is P331S.

179. The heterodimeric Fc variant according to any one of embodiments 93 to 103 and 165 to 178, wherein the second Fc polypeptide further comprises the mutation S267A or S267Q.

180. The heterodimeric Fc variant according to any one of embodiments 93 to 103 and 165 to 179, wherein the second Fc polypeptide further comprises the mutation V266L.

181. The heterodimeric Fc variant according to any one of embodiments 93 to 103 and 165 to 180, wherein the first Fc polypeptide further comprises a mutation at one or more of positions 235, 237, 239, 264, 266, 267, 269, 270, 271, 272, 273, 323, 326 and/or 332.

182. The heterodimeric Fc variant according to embodiment 181, wherein:
(i) the mutation at position 235 is selected from L235A, L235D, L235E, L235F, L235H, L235I, L235P, L235Q, L235S, L235T, L235V, L235W and L235Y;
(ii) the mutation at position 237 is selected from G237A, G237F, G237L, G237N, G237T, G237W and G237Y;
(iii) the mutation at position 239 is selected from S239A, S239D, S239E, S239G, S239I, S239L, S239N, S239Q, S239R and S239V;
(iv) the mutation at position 264 is selected from V264A, V264F, V264I, V264L and V264T;
(v) the mutation at position 266 is V266I;
(vi) the mutation at position 267 is selected from S267A, S267G, S267H, S267I, S267N, S267P, S267T and S267V;
(vii) the mutation at position 269 is selected from E269A, E269D, E269F, E269G, E269H, E269I, E269K, E269L, E269N, E269P, E269Q, E269R, E269S, E269T, E269V, E269W and E269Y;
(viii) the mutation at position 270 is selected from D270A, D270E, D270F, D270H, D270I, D270N, D270Q, D270S, D270T, D270W and D270Y;
(ix) the mutation at position 271 is selected from P271D, P271E, P271G, P271H, P271I, P271K, P271L, P271N, P271Q, P271R, P271V and P271W;
(x) the mutation at position 272 is selected from E272A, E272D, E272F, E272G, E272H, E272I, E272L, E272N, E272S, E272T, E272V, E272W and E272Y;
(xi) the mutation at position 273 is V273A;
(xii) the mutation at position 323 is selected from V323A, V323I and V323L;
(xiii) the mutation at position 326 is selected from K326A, K326D, K326H, K326N, K326Q, K326R, K326S and K326T, and
(xiv) the mutation at position 332 is selected from I332A, I332L, I332T and I332V.

183. The heterodimeric Fc variant according to embodiment 181 or 182, wherein the first Fc polypeptide further comprises a mutation at position 235.

184. The heterodimeric Fc variant according to embodiment 183, wherein the mutation at position 235 is L235D.

185. The heterodimeric Fc variant according to any one of embodiments 181 to 184, wherein the first Fc polypeptide further comprises a mutation at position 267.

186. The heterodimeric Fc variant according to embodiment 185, wherein the mutation at position 267 is S267A.

187. The heterodimeric Fc variant according to any one of embodiments 93 to 103 and 165 to 186, wherein the second Fc polypeptide further comprises a mutation at one or more positions selected from 234, 235, 237, 240, 264, 269, 271, 272 and 273.

188. The heterodimeric Fc variant according to embodiment 187, wherein:
(i) the mutation at position 234 is selected from L234A, L234D, L234E, L234F, L234G, L234I, L234N, L234P, L234Q, L234S, L234T, L234V, L234W and L234Y;
(ii) the mutation at position 235 is selected from L235A, L235D, L235F, L235G, L235H, L235N, L235W and L235Y;
(iii) the mutation at position 237 is selected from G237A, G237D, G237E, G237F, G237H, G237I, G237K, G237L, G237N, G237Q, G237R, G237S, G237T, G237V, G237W and G237Y.
(iv) the mutation at position 240 is selected from V240I, V240L and V240T;
(v) the mutation at position 264 is selected from V264L and V264T;
(vi) the mutation at position 269 is selected from E269D, E269T and E269V;
(vii) the mutation at position 271 is P271G;
(viii) the mutation at position 272 is selected from E272A, E272D, E272I, E272K, E272L, E272P, E272Q, E272R, E272T and E272V, and
(ix) the mutation at position 273 is selected from V273A, V273I, V273L and V273T.

189. The heterodimeric Fc variant according to embodiment 187 or 188, wherein the second Fc polypeptide further comprises a mutation at position 237.

190. The heterodimeric Fc variant according to embodiment 189, wherein the mutation at position 237 is G237D or G237L.

191. The heterodimeric Fc variant according to any one of embodiments 93 to 103 and 165 to 190, wherein amino acids 325 to 331 in the second Fc polypeptide are replaced with a polypeptide between 8 and 15 amino acids in length.

192. The heterodimeric Fc variant according to embodiment 191, wherein the polypeptide is derived from a loop-forming segment of a second protein, and wherein the loop-forming segment comprises:
(a) an amino acid sequence as set forth in any one of SEQ ID NOs: 4, 5, 6, 7, 8, 9, 10, 11, 12, 13 or 14, or
(b) an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations.

193. The heterodimeric Fc variant according to embodiment 93, wherein the heterodimeric Fc variant comprises the amino acid mutations as set out for any one of the variants shown in Table 6.23 or 6.26.

194. The heterodimeric Fc variant according to embodiment 93, wherein:
(i) the first Fc polypeptide comprises the mutations L234F_L235D_G236N_H268Q_A327G_A330K_P331S, and the second Fc polypeptide comprises the mutations G236D_G237L_S239D_V266L_S267A_H268D (Variant 31190);
(ii) the first Fc polypeptide comprises the mutations L234F_G236N_H268Q_A327G_P329I_A330K_P331S, and the second Fc polypeptide comprises the mutations G236D_G237D_S239D_V266L_S267A_H268D (Variant 31256);
(iii) the first Fc polypeptide comprises the mutations L234F_G236N_H268Q_A327G_P329A_A330K_P331S, and the second Fc polypeptide comprises the mutations G236D_G237L_S239D_V266L_S267A_H268D (Variant 32274);
(iv) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(v) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(vi) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(vii) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294); or
(viii) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

195. The heterodimeric Fc variant according to embodiment 93, wherein:
(a) the first Fc polypeptide comprises the mutation G236N, and a mutation at one or more positions selected from 234, 268, 327, 330 and 331, wherein:
   (i) the mutation at position 234 is selected from L234A, L234F, L234G, L234H, L234I, L234N, L234P, L234Q, L234S, L234T, L234V, L234W and L234Y,
   (ii) the mutation at position 268 is selected from H268A, H268D, H268E, H268F, H268G, H268I, H268K, H268L, H268N, H268P, H268Q, H268R, H268S, H268T, H268V, H268W and H268Y,
   (iii) the mutation at position 327 is selected from A327G and A327E;
   (iv) the mutation at position 330 is selected from A330K, A330H, A330Q, A330R, A330S and A330T, and
   (v) the mutation at position 331 is selected from P331A, P331D, P331E, P331H, P331Q and P331S, and
(b) the second Fc polypeptide comprises:
   (i) the mutation G236D;
   (ii) replacement of the native loop at positions 325 to 331 with a polypeptide of between 8 and 15 amino acids in length, wherein the polypeptide is derived from a loop-forming segment of a second protein, and wherein the loop-forming segment comprises an amino acid sequence as set forth in any one of SEQ ID NOs: 4, 5, 6, 7, 8, 9, 10, 11, 12, 13 or 14, or a variant thereof comprising 1, 2, 3, 4 or 5 amino acid mutations, and
   (iii) one or more mutations selected from S239D, S239E, V266I, S267I, S267Q, S267V and H268D.

196. The heterodimeric Fc variant according to embodiment 195, wherein the second Fc polypeptide comprises:
(i) the mutation G236D;
(ii) replacement of the native loop at positions 325 to 331 with a polypeptide of between 8 and 15 amino acids in length, wherein the polypeptide is derived from a loop-forming segment of a second protein, and wherein the loop-forming segment comprises an amino acid sequence as set forth in any one of SEQ ID NOs: 4, 5, 6, 7, 8, 9, 10, 11, 12, 13 or 14, or a variant thereof comprising 1, 2, 3, 4 or 5 amino acid mutations, and
(iii) the mutation S239D or S239E, and/or the mutation H268D, and/or the mutation S267I or S267V.

197. The heterodimeric Fc variant according to embodiment 195, wherein the second Fc polypeptide comprises:
(i) the mutation G236D;
(ii) replacement of the native loop at positions 325 to 331 with a polypeptide of between 8 and 15 amino acids in length, wherein the polypeptide is derived from a loop-forming segment of a second protein, and wherein the loop-forming segment comprises an amino acid sequence as set forth in any one of SEQ ID NOs: 4, 5, 6, 7, 8, 9, 10, 11, 12, 13 or 14, or a variant thereof comprising 1, 2, 3, 4 or 5 amino acid mutations, and
(iii) the mutations S239D, H268D and S267V.

198. The heterodimeric Fc variant according to any one of embodiments 195 to 197, wherein the mutation at position 234 in the first Fc polypeptide is L234F.

199. The heterodimeric Fc variant according to any one of embodiments 195 to 198, wherein the mutation at position 268 in the first Fc polypeptide is H268Q.

200. The heterodimeric Fc variant according to any one of embodiments 195 to 199, wherein the mutation at position 327 in the first Fc polypeptide is A327G.

201. The heterodimeric Fc variant according to any one of embodiments 195 to 200, wherein the mutation at position 330 in the first Fc polypeptide is A330K or A330T.

202. The heterodimeric Fc variant according to any one of embodiments 195 to 201, wherein the mutation at position 331 in the first Fc polypeptide is P331S.

203. The heterodimeric Fc variant according to embodiment 195, wherein:
(i) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(ii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(iii) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(iv) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294); or
(v) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

204. The heterodimeric Fc variant according to any one of embodiments 93 to 203, wherein the first Fc polypeptide and second Fc polypeptide further comprise one or more mutations selected from: A287F, T250V, L309Q and M428F.

205. The heterodimeric Fc variant according to embodiment 204, wherein the first Fc polypeptide and second Fc polypeptide further comprise the mutations A287F/M428F, A287F/T250V, M428F/T250V or T250V/L309Q.

206. The heterodimeric Fc variant according to any one of embodiments 93 to 205, wherein the heterodimeric Fc variant is a variant of an IgG1 Fc.

207. The heterodimeric Fc variant according to embodiment 206, wherein the heterodimeric Fc variant is a variant of a human IgG1 Fc.

208. The heterodimeric Fc variant according to any one of embodiments 93 to 207, wherein the selectivity of binding to FcγRIIb of the heterodimeric Fc variant is increased by at least 1.5-fold, or at least 2-fold, over the parental Fc region, and wherein:

Fold Increase in FcγRIIb Selectivity=Fold Difference in FcγRIIb Affinity/Fold Difference in FcγRIIaR Affinity,

wherein:

Fold Difference in FcγRIIb Affinity=K_{D} FcγRIIb (parental)/K_{D} FcγRIIb (variant),

and

Fold Difference in FcγRIIaR Affinity=K_{D} FcγRIIaR (parental)/K_{D} FcγRIIaR (variant).

209. The heterodimeric Fc variant according to any one of embodiments 93 to 208, wherein the heterodimeric Fc variant has increased binding affinity for FcγRIIb as compared to the parental Fc region.

210. The heterodimeric Fc variant according to embodiment 209, wherein the binding affinity of the heterodimeric Fc variant for FcγRIIb is increased by at least 10-fold over the parental Fc region, and wherein:

Fold Difference in FcγRIIb Affinity=K_{D} FcγRIIb (parental)/K_{D} FcγRIIb (variant).

211. A polypeptide comprising the heterodimeric Fc variant according to any one of embodiments 93 to 210 and one or more proteinaceous moieties fused or covalently attached to the heterodimeric Fc variant.

212. The polypeptide according to embodiment 211, wherein the polypeptide is an antibody and the one or more proteinaceous moieties are one or more antigen-binding domains.

213. The polypeptide according to embodiment 212, wherein at least one of the antigen-binding domains binds to a tumour-associated antigen or tumour-specific antigen.

214. A pharmaceutical composition comprising the heterodimeric Fc variant according to any one of embodiments 93 to 210, or the polypeptide according to any one of embodiments 211 to 213, and a pharmaceutically acceptable carrier or diluent.

215. A polypeptide according to any one of embodiments 211 to 213 for use in therapy.

216. A polypeptide according to embodiment 213 for use in the treatment of cancer.

217. Nucleic acid encoding the heterodimeric Fc variant according to any one of embodiments 93 to 210, or the polypeptide according to any one of embodiments 211 to 213.

218. A host cell comprising the nucleic acid according to embodiment 217.

219. A method of preparing the heterodimeric Fc variant according to any one of embodiments 93 to 210, or the polypeptide according to any one of claims 211 to 213, the method comprising expressing nucleic acid encoding the heterodimeric Fc variant or the polypeptide in a host cell.

The following Examples are provided for illustrative purposes and are not intended to limit the scope of the disclosure in any way.

### EXAMPLES

### Overview

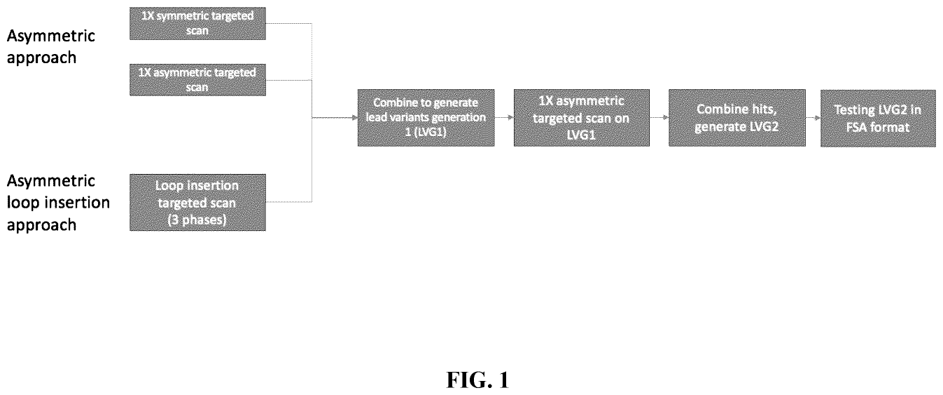

FIG. 1 provides an overview of the strategy employed to generate FcγRIIb specific variants. The various steps are described in detail in the following Examples. Briefly, two approaches were employed to identify initial variants showing greater selectivity for FcγRIIb than the wild-type IgG1 Fc. Variants from each of these approaches were then combined and the resulting variants further refined to generate optimized FcγRIIb selective variants. Both of these approaches leveraged the asymmetric nature of the interaction of the Fc region with FcγRIIb and thus required a heterodimeric Fc as a starting scaffold such that the two chains of the Fc could be distinguished.

The two approaches employed in identifying the initial variants were:

(1) Asymmetric 1× approach (FIG. 2A): in this approach, mutations in the CH2 and hinge region were screened to take advantage of the asymmetric nature of the interaction of the Fc region with FcγRIIb.

(2) Loop replacement approach (FIG. 2B): in this approach, Loop 3 (L3) on one chain of the Fc region was replaced and extended. The L3 loop normally is too far from FcγRIIb to be involved in binding (see FIG. 2B). The effect of the loop replacement approach was to extend this region such that it was in closer proximity to position 135 in FcγRIIb. The amino acid at position 135 in FcγRIIb is serine (S), whereas in FcγRIIa, the amino acid in the corresponding position is leucine (L). Creating an additional interaction at this position resulted in an improved selectivity of the Fc for FcγRIIb.

The overall strategy described herein provided a library of variants having increased FcγRIIb selectivity. The variants have a range both FcγRIIb selectivities and FcγRIIb affinities and demonstrate various effector profiles. The library thus allows for selection of a variant with the best activity profile for a given application.

### General Methods

### Preparation of Variants

Variants and controls were prepared by site-directed mutagenesis and/or restriction/ligation using standard methods. The final DNA was sub-cloned into the vector pTT5 (see U.S. Pat. No. 9,353,382). The following scaffolds were used for preparation of the variants:

Scaffold 1: Full-size antibody (FSA) based on trastuzumab with a homodimeric IgG1 Fc.

Scaffold 2: One-armed antibody (OAA) scaffold comprising one trastuzumab Fab and a heterodimeric IgG1 Fc comprising the following mutations:
Chain A: T350V_L351Y_F405A_Y407V
Chain B: T350V_T366L_K392L_T394W

The relevant sequences are provided below.

Heavy chain A, upper hinge, CH2 and CH3 domains:
[SEQ ID NO: 2]
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYVYPPSRDELTKNQ
VSLTCLVKGFYPSDIAVEWESNGQPENNYKTTPPVLDSDGSFALVSKLT
VDKSRWQQGNVFSCSVMHEALHNHYTQKSLSLSPG
Heavy chain B, upper hinge, CH2 and CH3 domains:
[SEQ ID NO: 3]
EPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYVLPPSRDELTKNQ
VSLLCLVKGFYPSDIAVEWESNGQPENNYLTWPPVLDSDGSFFLYSKLT
VDKSRWQQGNVFSCSVMHEALHNHYTQKSLSLSPG

Scaffold 3: Full-size antibody (FSA) based on trastuzumab comprising the same heterodimeric Fc as for Scaffold 2.

Scaffold 4: Full-size antibody (FSA) based on the 4G7 anti-CD19 antibody (Meeker, et al., 1984, Hybridoma, 3:305-320) comprising the same heterodimeric Fc as for Scaffold 2. Sequences used were as described in U.S. Pat. No. 8,524,867.

Scaffold 5: Full-size antibody (FSA) based on the CP-870,893 anti-CD40 antibody (Gladue, et al., 2011, Cancer Immunol Immunother, 60:1009-1017) comprising the same heterodimeric Fc as for Scaffold 2. Variable domain sequence was obtained from International Patent Application Publication No. WO 2013/132044.

### Expression—Protocol 1

Expression was carried out in 2 mL, 50 mL or 500 mL CHO 3E7 cells. CHO cells were transfected in exponential growth phase (1.5 to 2 million cells/mL) with aqueous 1 mg/mL 25 kDa polyethylenimine (PEI^{pro}, Polyplus Transfection SA, Illkirch, France) at a PEI:DNA ratio of 2.5:1 (Delafosse, et al., 2016, J. Biotechnol., 227:103-111). DNA was transfected in pre-determined optimal DNA ratios of the heavy chain A (HC-A), light chain (LC), and heavy chain B (HC-B) that allow for heterodimer formation (e.g., HC-A/HC-B/LC ratios=25:25:50%). Transfected cells were harvested after 5-6 days. Culture medium was collected after centrifugation at 4000 rpm and clarified using a 0.45 m filter.

The clarified culture medium was loaded onto a MabSelect™ SuRe™ (GE Healthcare, Baie-d'Urfé, QC, Canada) Protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. The Protein-A purified antibody was further purified by size exclusion chromatography (SEC). For gel filtration, 3.5 mg of the antibody mixture was concentrated to 1.5 mL and loaded onto a Sephadex 200 HiLoad® 16/600 200 pg column (GE Healthcare) equilibrated in PBS pH 7.4 via an AKTA Express FPLC at a flow-rate of 1 mL/min. Fractions corresponding to the purified antibody were collected, concentrated to ˜1 mg/mL and stored at −80° C.

### Expression—Protocol 2

Expression was performed using HEK 293-6E cells (NRC, Canada) on either small-scale (1 mL) or large-scale (30 mL or greater).

For 1 mL-scale expressions, HEK 293-6E cells were transfected in exponential growth phase (1.5 to 2.0 million cells/mL) with 1 μg DNA/mL cells using DNA pre-complexed with the cationic lipid 293Fectin™ (Life Technologies, Paisley, U.K.). Heavy chain and light chain DNA were mixed at a ratio of 47.5:52.5% and DNA was complexed with 293Fectin™ at final concentrations of 11.7 μg/mL DNA, 1.65% (v/v) 293Fectin™ then incubated at ambient temperature for 30 min before addition to cells. In order to achieve optimal heterodimer formation, the ratio of the heavy chain A and heavy chain B DNA of transfection mixes was either 50:50%, or a small variation thereof. Cells were cultured for 5-6 days in a humidified shaking incubator at 37° C. and 5% carbon dioxide in a 96-well deep well plate sealed with a gas-permeable seal. Culture medium was then collected after centrifugation at 1600×g.

For large-scale expressions, HEK 293-6E cells were transfected in exponential growth phase (1.5 to 2.0 million cells/mL) with 1 μg DNA/mL cells using DNA pre-complexed with a Gemini cationic lipid (Camilleri et al., 2000, Chem. Commun., 1253-1254). Heavy chain and light chain DNA were mixed at a ratio of 50:50% and DNA was complexed with Gemini at final concentrations of 10 μg/mL DNA, 40 μg/mL Gemini then incubated at ambient temperature for 15-30 min before addition to cells. Heavy chain A and heavy chain B DNA ratios of transfection mixes were as described above. Cells were cultured for up to 10 days in a humidified shaking incubator at 37° C. and 5% carbon dioxide in an appropriately sized Erlenmeyer flask or BioReactor tube. Culture medium was then collected after centrifugation at 2750×g and clarified using a 0.22 μm filter.

The clarified culture medium was loaded onto a MabSelect™ SuRe™ (GE Healthcare, Little Chalfont, U.K.) protein A column, washed with 3-10 column volumes of Tris-Acetate buffer at pH7.5, then eluted with 2-5 column volumes of acetic acid at pH 2.6 with the elution fraction neutralized with TRIS. Further purification by size exclusion chromatography (Superdex™ 200 column (GE Healthcare, Little Chalfont, U.K.) with PBS running buffer) and/or cationic exchange (ReSource™ S column (GE Healthcare, Little Chalfont, U.K.)) was utilised on selected samples. Protein-A purified antibodies were buffer-exchanged into PBS.

### Preparation of Fcγ Receptors

### Protocol 1

FcγRIIaH, IIaR, IIb, IIIaF and IIIaV were produced in HEK 293-6E cells and FcγRIa was produced in CHO-3E7 cells as described previously (Dorian-Thibaudeau, et al., 2014, J. Immunol. Methods, 408:24-34). The human FcRn was also expressed in HEK 293-6E cells by the co-transfection of the alpha subunit (p51) extracellular domain containing a TEV-cleavable C-terminal His-tag with β2-microglobulin in a 1:1 ratio. Following purification as described in Dorion-Thibaudeau et al. (ibid.) the C-terminal His-tag was removed by TEV cleavage.

### Protocol 2

Soluble FcγRI extracellular domain with a C-terminal 6×His tag was purchased from R&D Systems (Catalogue number 1257-Fc). Soluble FcγRIIaH, IIaR, IIb, IIIaF and IIIaV extracellular domains were produced in HEK 293-6E cells with C-terminal 10×His tags. Cells were transfected in exponential growth phase (1.5 to 2.0 million cells/mL) with 1 μg DNA/mL cells using DNA pre-complexed with a Gemini cationic lipid (Camilleri et al., 2000, Chem. Commun., 1253-1254.). Cells were cultured for up to 7 days in a humidified shaking incubator at 37° C. and 5% carbon dioxide in an appropriately sized Erlenmeyer flask. The time of harvest was determined by when the cell viability dropped below 50%. Culture medium was then collected after centrifugation at 2750×g and clarified using a 0.22 μm filter.

The clarified culture medium was buffer-exchanged by dialysis or tangential flow filtration into pH7.7 load buffer containing 25 mM imidazole and applied to a Ni-Sepharose 6 column (GE Healthcare, Little Chalfont, U.K.), then eluted by increasing the buffer imidazole concentration to 300 mM. Eluted protein was concentrated and buffer-exchanged into PBS by dia-filtration then further purified by size exclusion chromatography (Superdex® 75 column (GE Healthcare, Little Chalfont, U.K.))

Soluble human FcRn extracellular domain was expressed in HEK 293-6E cells by the co-transfection of the alpha subunit containing a C-terminal 6×His-tag with β2 microglobulin at a 1:1 ratio and expressed as otherwise described for the FcγRs. The pH of the clarified culture medium was adjusted to pH5.3 with citrate then loaded onto an IgG Sepharose column (GE Healthcare, Little Chalfont, U.K.). Bound protein was eluted with pH7.7 HEPES buffer. Eluted protein was concentrated and buffer-exchanged into PBS by dia-filtration then further purified by size exclusion chromatography (Superdex® 75 column (GE Healthcare, Little Chalfont, U.K.))

Soluble FcγRIIb and FcγRIIaR extracellular domains genetically fused via the C-terminus to human IgG1 Fc containing the CH2 mutations L234A_L235A_D265S to remove interaction between the FcγR and Fc domains were expressed as described above for the His-tagged extracellular domains. The clarified culture medium was loaded onto a MabSelect™ SuRe™ protein A column (GE Healthcare, Little Chalfont, U.K.), washed with 3-10 column volumes of Tris-Acetate buffer at pH7.5, then eluted with 2-5 column volumes of acetic acid at pH 2.6 with the elution fraction neutralized with TRIS. Samples were then buffer-exchanged into PBS and purified further by size exclusion chromatography (Superdex® 200 column (GE Healthcare, Little Chalfont, U.K.) with PBS running buffer.

### Fcγ Receptor Binding: Surface Plasmon Resonance (SPR)

### Protocol 1

Affinity of FcγRs for antibody Fc was measured by SPR using a ProteOn™ XPR36 at 25° C. with PBS containing 150 mM NaCl, 3.4 mM EDTA, and 0.05% Tween 20 at pH 7.4 as the running buffer. For trastuzumab variants, recombinant HER2 was immobilized on a GLM sensorchip using standard amine coupling with a BioRad amine coupling kit. Briefly, the GLM sensorchip was activated with NHS/EDC followed by injecting HER2 at 4.0 μg/mL in 10 mM NaOAc (pH 4.5) until approximately 3000 resonance units (RUs) were immobilized. The remaining active groups were quenched with ethanolamine. Wild-type trastuzumab variants were then indirectly captured onto the SPR surface by injecting a 40 μg/mL solution purified antibody in the ligand direction at 25 μL/min for 240s resulting in approx. 500 RUs on the surface. Following buffer injections to establish a stable baseline in the analyte direction, analyte was injected at 50 μL/min for 120s with a 180s dissociation phase to obtain a set of binding sensorgrams. Five concentrations of a 3-fold dilution series of the FcγRs with 10 μM top nominal concentrations for all receptors were used except 30 nM for FcγR1a, and buffer was included for double referencing. Resultant Kd (affinity) values were determined from the aligned and referenced sensorgrams using the Equilibrium Fit model in ProteOn™ Manager v3.1.0 with reported values as the mean of two or three independent runs.

### Protocol 2

Affinity of FcγRs for antibody Fc was measured by SPR using a Biacore™ 4000 (GE Healthcare, Little Chalfont, U.K.) at 25° C. with PBSTE (PBS with 0.05% Tween-20 and 3.4 mM EDTA) as the running buffer. For anti-HER2 antibodies, a CM5 chip (GE Healthcare, Little Chalfont, U.K.) was immobilized with recombinant HER2 extracellular domain (Merck, Darmstadt, Germany or ThermoFisher Scientific, Loughborough, U.K.) utilizing amine coupling (EDC/NHS chemistry). Briefly, the CM5 sensorchip was activated with NHS/EDC followed by injection of HER2 at 10.0 μg/mL in 10 mM NaOAc (pH 4.5). Immobilization levels ranged between 1000-4000 RU. Any remaining active groups were then quenched with ethanolamine. Antibodies were first captured on the immobilized surface of the chip by injecting at approximately 15 μg/ml across the spots and flow cells for 35 s at a flow-rate of 10 μl/min, leaving spot 3 blank for reference subtraction. Receptors were diluted in PBSTE buffer to a defined concentration range that was dependent on their expected affinity. Six concentrations were used per analyte including zero. Analyte contact time was optimized dependent on the receptor used and its expected kinetics. For example, for FcγRIIb and FcγRIIaR contact time was 18s at 30 μl/min. The chip surface was regenerated after each analyte concentration injection with 87 mM phosphoric acid. Prior to testing, the chip was prepared with 3×18s injections of 87 mM phosphoric acid. Double reference subtraction was performed (reference spot 3 and 0 receptor concentration) and binding responses were normalised by the antibody capture level. Samples were analysed using either kinetics and/or steady state (equilibrium) fit models.

### FcγRIIb Binding and Selectivity: Competition Electrochemiluminescence Assay

The relative affinity of Fc variants to FcγRIIb and the relative selectivity of Fc variants to FcγRIIb as compared to FcγRIIaR was measured by competition electrochemiluminescence assay using a MSD SECTOR 6000 Imager (Meso Scale Diagnostics, Rockville, U.S.A.). MSD standard bind 384-well plates were coated with 10 nM soluble HER2 extracellular domain (Speed Biosystems, Gaithersburg, U.S.A.) in PBS overnight at 4° C. then blocked with 3% bovine serum albumin (Sigma Aldrich, Gillingham, U.K.) in PBS containing 0.05% Tween-20 for 1 h. Test antibody variants were applied to the plate at 100 nM in PBS containing 0.5% BSA, 0.05% Tween-20 (assay buffer) and allowed to bind for 1 h. After washing, biotinylated FcγRIIb extracellular domain-Fc fusion in assay buffer was added to each sample well and incubated for 1h in the presence or absence of FcγRIIaR extracellular domain-Fc fusion. After washing, a 1:2000 dilution of Streptavidin-sulphoTag (Meso Scale Diagnostics) in assay buffer was added to each sample and the plate incubated for 60 min. The plate was washed again, 1× Read Buffer T (Meso Scale Diagnostics) added to each well and the plate read immediately. Data were analysed as both the signal of sample incubated with the biotinylated FcγRIIb-Fc receptor alone relative to a control (considered a measure of the relative affinity for FcγRIIb) and the proportion of this signal measured in the presence of the non-biotinylated FcγRIIaR-Fc (considered a measure of the selectivity of Fc variants for FcγRIIb over FcγRIIaR). Experiments were performed as both dose-response curves, in which the FcγRIIb-Fc concentration was held constant and FcγRIIaR-Fc concentration varied, or as “single-shot” assays at single FcγRIIb-Fc and FcγRIIaR-Fc concentrations. For the screening of large numbers of variants, the concentration of receptors used in single-shot assays was 10 nM biotinylated FcγRIIb-Fc and 100 nM FcγRIIaR-Fc.

### FcRn Binding

Affinity of FcRn for antibody Fc was measured by SPR using a Biacore™ T200 (GE Healthcare, Little Chalfont, U.K.) at 25° C. with HBS-EP+ pH 7.4 or MES pH 6.0 as the running buffer. Samples were captured on an immobilized protein L CM5 chip (GE Healthcare), but 4G7 anti-CD19 antibodies failed to capture. Antibodies were first captured on the immobilized surface of the chip by injecting at approximately 15 μg/ml across the spots and flow cells for 60s at a flowrate of 5 μl/min. The receptor was diluted to a defined concentration range in HBS-EP+pH 7.4 or MES pH 6.0 buffer. Three concentrations (4096, 512 and 0 nM) were used per analyte at pH 7.4 and four (512, 64, 8 and 0 nM) were used per analyte at pH 6.0. The chip surface was regenerated after each analyte concentration injection with 10 mM glycine pH 1.5. Results were analysed using Biacore™ T200 Evaluation V2 software and 1:1 binding kinetics model.

### Differential Scanning Calorimetry (DSC)

### Protocol 1

Each antibody construct was diluted to 0.2 mg/mL in PBS, and a total of 400 μL was used for DSC analysis with a VP-Capillary DSC (GE Healthcare). At the start of each DSC run, five buffer blank injections were performed to stabilize the baseline, and a buffer injection was placed before each antibody injection for referencing. Each sample was scanned from 20-100° C. at a 60° C./h rate, with low feedback, 8s filter, 5 min preTstat, and 70 psi nitrogen pressure. The resulting thermograms were referenced and analyzed using Origin 7 software (OriginLab Corporation, Northampton, MA).

### Protocol 2

Antibody constructs were assessed by the same method as described for Protocol 1 above except that antibody concentrations of 0.1-1.0 mg/ml were used, with concentrations of 0.4 mg/ml or greater preferred.

### Differential Scanning Fluorimetry (DSF)

20 μL of purified sample (between 0.2 and 1.0 mg/mL) was added to 10 μL of SYPRO® Orange (Invitrogen, Paisley, U.K.), diluted from 5000× stock to 20× with reverse osmosis (RO) water and placed in a clear walled 96-well PCR plate. Samples were incubated at 40° C. for 5 min, then the fluorescence emission of the SYPRO® Orange was measured using a BioRad CFX Connect™ RT-PCR machine (BioRad, Watford, U.K.) between 40-95° C. using a 15° C./h rate. Bio-Rad CFX Manager™ version 3.1 was used to analyse the peaks and derive temperatures of protein unfolding events which were then correlated to the unfolding of known domains within the protein.

### Size Exclusion Chromatography (SEC)

10 μL of purified sample (within a concentration range of between 0.2 and 2 mg/mL) was injected onto a Supelco TSKgel® G3000 SWXL size exclusion column (Tosoh, Reading, U.K.) using an Agilent 1100 HPLC system (Agilent, Stockport, U.K.) flowing 400 mM sodium phosphate, 200 mM NaCl, pH 6.8 mobile phase at a constant 0.5 mL/minute with a run time of 30 minutes per sample. A diode array detector was connected in line of the flow after the column and the UV/vis absorption at 210 and 280 nm recorded. The resultant traces were integrated using Chemstation software (Agilent, Stockport, U.K.) and subsequently analyzed using ChromView™ software. Sample purity was recorded by categorization of % area main peak compared to total % area of peaks with a higher molecular weight than main peak and total % area of peaks with a lower molecular weight than main peak.

### C1q Binding

Binding of antibody constructs to human C1q was evaluated by ELISA. Test antibody constructs were coated onto wells of a 96-well flat-bottomed Nunc Maxisorp™ plate (Invitrogen, Paisley, U.K.) by addition of 100 μl of 10 μg/ml test antibody in PBS per well. Plates were sealed and incubated at 4° C. for 16h. Plates were washed 3 times with 300 μl of PBS containing 0.05% (v/v) Tween-20. The plate surface was then blocked by addition of 200 μl of 1% (w/v) bovine serum albumin per well. Plates were incubated at ambient temperature for 1 h then washed as before. Recombinant human C1q (C1740, Sigma Aldrich, Gillingham, U.K.) was diluted in 50 mM carbonate/bicarbonate buffer (C3041, Sigma Aldrich) to final assay concentrations and 100 μl added per well. Samples were incubated for 2h at ambient temperature and plates were washed as before. 100 μl of sheep anti-human C1q-HRP (Ab46191, AbCam, Cambridge, U.K.) diluted with PBS to 2 μg/ml was then added per well, samples incubated at ambient temperature for 1 h, then plates washed as before. For detection, 100 μl of Sureblue™ TMB (52-00-01, Seracare Life Sciences Inc., Milford, MA) was added per well and samples incubated with agitation for 20 min at ambient temperature. Reactions were stopped by addition of 100 μl of 1M HCl to each well. Absorbance of each sample well was then measured at 450 nm using a M5e SpectraMax® plate reader (Molecular Devices, Wokingham, U.K.). For each antibody variant, seven C1q concentrations from 2 μg/ml to 6 ng/ml in half-log steps plus a no C1q control were tested in duplicate. Data were analyzed using Prism (GraphPad, San Diego, CA). Binding curves were fitted using a 4-parameter non-linear regression model of the absorbance and log-transformed C1q concentration. Concentration of C1q at which binding exceeded a threshold absorbance (0.5 OD, 17% of maximum signal) was interpolated from the fitted curve. For screening, comparisons between samples were made based on the signal at 2 μg/ml C1q. Data were normalized as the % of WT.

### Stress Test

Concentration normalized samples were stressed for 2 weeks at 40° C. (stressed condition) or 4° C. (non-stressed condition) in both an acidic and a neutral buffer. After this time, the 40° C. sample was returned to 4° C. Stressed and non-stressed samples were evaluated for changes in aggregation and fragmentation by analytical SEC and for change in binding to FcγRIIb by SPR.

Aggregation and fragmentation was evaluated using a SEC method similar to that described above. Briefly, 10 μL of purified sample (at a concentration of 1 mg/mL) was injected onto an ACQUITY™ UPLC™ Protein BEH 200 4.6×150 mm size exclusion column (Waters Corporation, Elstree, U.K.) using an Agilent 1100 HPLC system (Agilent, Stockport, U.K.) flowing 100 mM sodium phosphate, 350 mM NaCl, pH 6.8 as mobile phase. A diode array detector was connected in line of the flow after the column and the UV/vis absorption at 214 and 280 nm was recorded.

Binding of samples to FcγRIIb antigen was evaluated by SPR using a Biacore™ 8K+ (GE Healthcare, Little Chalfont, U.K.) at 25° C. The method utilizes the Rₘₐₓ binding signal of antigen binding to captured antibody to evaluate the effective concentration of active sample by comparing this signal to the signal of a standard curve of a representative sample captured at different concentrations. For the data reported herein, the reference antibody was a heterodimeric anti-CD19 antibody with symmetric E233D_G237D_P238D_H268D_P271G_A330R CH2 mutations which was evaluated over a concentration range of 2.5-20 μg/ml. Test samples were each evaluated at a concentration of 10 μg/ml. Antibodies were captured onto a Sensor Chip Protein A (GE Healthcare, Little Chalfont, U.K.) chip surface by injection at 10 μl/min for 60s. 20 μg/ml FcγRIIb was then injected over the chip at 30 μl/min for 60s. The Rₘₐₓ of each injection was reported. Values for the reference antibody were used to generate standard curves for both the antibody capture and antigen binding steps. Rₘₐₓ values for test samples were then interpolated from the standard curve and multiplied by the dilution factor required to dilute the sample from its original concentration to 10 ug/ml to give estimates of antibody concentration (from the antibody capture step) and relative antigen binding concentration (from the antigen binding step). Loss of binding activity was calculated by the difference in relative antigen binding concentration of a sample under stressed and unstressed conditions.

### Example 1: Asymmetric Point Mutations

### 1.1 1× Symmetric Mutations

Based on in silico analysis of the structure of the IgG1 Fc region bound to different Fcγreceptors, the lower hinge residues were identified as potential sites for introducing mutations to modify FcγR affinity and selectivity. Variants comprising selected mutations in this region were constructed in a symmetric homodimeric scaffold (Scaffold 1) and affinity and selectivity of these variants for FcγRIIb, FcγRIIaR, FcγRIIaH and FcγRIIIa were determined experimentally by SPR (see General Methods, Protocol 1).

Table 1.1 shows the top mutations identified in this screen. G236 was identified as the most promising position in the lower hinge for introducing mutations to drive FcγRIIb selectivity.

TABLE 1.1
Affinity and Selectivity of Top Mutations
Identified in 1X Symmetrical Screen

| | K_{a, mut}/K_{a, WT} | | | | IIb/IIaR |
|---|---|---|---|---|---|
| Mutation | FcγRIIbY | FcγRIIaH | FcγRIIaR | FcγRIIIa | Selectivity |
| G236K | 0.26 | 0.06 | 0.07 | 0.10 | 3.99 |
| G236N | 0.31 | 0.24 | 0.12 | 0.02 | 2.46 |
| L234Q | 0.20 | 0.07 | 0.08 | 0.18 | 2.43 |
| L235P | 0.42 | 0.16 | 0.19 | 0.45 | 2.22 |
| L234G | 0.10 | 0.04 | 0.05 | 0.06 | 2.10 |
| L235S | 0.26 | 0.17 | 0.13 | 0.25 | 2.00 |
| L235V | 0.47 | 0.42 | 0.24 | 0.45 | 1.93 |
| L234D | 0.43 | 0.08 | 0.23 | 0.20 | 1.90 |
| L235A | 0.40 | 0.23 | 0.21 | 0.42 | 1.87 |

### 1.2 Asymmetric Simple CH2 Mutations

### 1) System Analysis of the Fc/FcγRIIb Interface

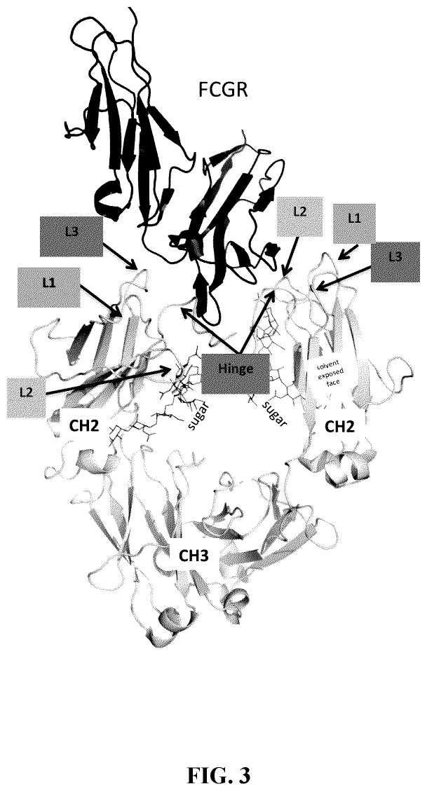

A crystal structure of the complex comprising IgG1 Fc bound to FcγRIIb was used to create a model amenable for in silico systematic screening. A cartoon representation of this model is shown in FIG. 3.

A number of in silico metrics were used to perform a systematic system analysis of the interface between the Fc region and the FcγRIIb, including sequence score, residue contacts and affinity decomposition. Sequence score is based on the sequence identity of a given residue across the CH2 domains of different species and isotypes, with high sequence scores being assigned to residues with high sequence conservation across species and isotypes. Residues with high sequence scores are often important for function, protein folding/stability or both. Residue contacts evaluates the interconnectivity between residues. Residues located at the interface that are highly connected are deemed to be hot spots (‘H’), whereas those located at the interface but with little connectivity are deemed cold spots (‘C’). Affinity decomposition quantifies in energy terms (kcal/mol⁻¹) the contribution of each residue to the Fc/FcγRIIb complex. Residues with negative energies strengthen the complex, whereas high positive energies reflect repulsion between the residue and FcγRIIb.

The results of the system analysis are shown in Table 1.2.

TABLE 1.2
Analysis of Residues at the Fc/FcγRIIb Interface

| | | Sequence | Chain A | | Chain B | |
|---|---|---|---|---|---|---|
| | Wild-type | conservation | Residue | Affinity | Residue | Affinity |
| Region | residue | score | contacts | decomp | contacts | decomp |
| Hinge | 234.LEU | 6 | | | | −0.3 |
| Hinge | 235.LEU | 14 | | 0.5 | | −2.1 |
| Hinge | 236.GLY | 24 | C | 0.9 | | −0.9 |
| Hinge | 237.GLY | 34 | | −1.4 | C | −2.0 |
| Hinge | 238.PRO | 62 | | −0.7 | | −0.6 |
| S1 | 239.SER | 100 | | −2.1 | H | −0.4 |
| S1 | 240.VAL | 79 | | −0.1 | | 0.0 |
| S1 | 241.PHE | 89 | | −0.2 | | 0.0 |
| S2 | 263.VAL | 100 | | 0.1 | | 0.0 |
| S2 | 264.VAL | 88 | | −0.4 | | 0.0 |
| L1 | 265.ASP | 89 | | −7.5 | | 0.7 |
| L1 | 266.VAL | 67 | C | −0.4 | | 0.1 |
| L1 | 267.SER | 36 | H | −0.4 | | 0.1 |
| L1 | 268.HIS | 32 | | −0.7 | | 0.4 |
| L1 | 269.GLU | 56 | H | −0.2 | | 0.0 |
| L1 | 270.ASP | 28 | | −10.4 | | −0.1 |
| L1 | 271.PRO | 61 | | −0.2 | | 0.0 |
| S3 | 273.VAL | 100 | | 0.1 | | 0.0 |
| S4 | 292.ARG | 43 | C | 1.1 | | 0.1 |
| S4 | 293.GLU | 68 | | −0.4 | | 0.0 |
| L2 | 294.GLU | 60 | | 0.2 | | −0.1 |
| L2 | 295.GLN | 63 | | −0.1 | | 0.0 |
| L2 | 296.TYR | 17 | | −4.6 | | 0.0 |
| L2 | 298.SER | 50 | H | −1.6 | | 0.1 |
| S5 | 299.THR | 100 | H | −1.6 | | 0.0 |
| S5 | 300.TYR | 34 | C | −0.3 | | 0.0 |
| S5 | 301.ARG | 90 | | 0.7 | | 0.2 |
| S6 | 323.VAL | 100 | | 0.0 | | 0.0 |
| L3 | 325.ASN | 56 | | −0.1 | | 0.1 |
| L3 | 327.ALA | 25 | | 0.0 | | 0.0 |
| L3 | 328.LEU | 71 | | 0.0 | H | −2.0 |
| L3 | 329.PRO | 89 | | 0.0 | H | −6.1 |
| L3 | 330.ALA | 55 | | 0.0 | | −0.9 |
| L3 | 331.PRO | 69 | | 0.1 | | 0.0 |
| L3 | 332.ILE | 60 | | 0.0 | | −0.2 |

2) In silico 1× Scan

A systematic 1× scan was carried out in silico to identify residues that could increase the selectivity of the Fc region for FcγRIIb. A large number of metrics were simultaneously assessed, including the AMBER energy, which is a combination of van der Waals (VdW) and Coulombic interactions, and a knowledge-based potential metric that reflects the likelihood of residues to be in the same environment based on what is known from a large database such as that of the Protein Data Bank (PDB).

Table 1.3 summarizes the positions identified by this approach as being potentially useful, together with mutations at these positions that produced favourable metrics in silico for selectivity for FcγRIIb over FcγRIIaR.

TABLE 1.3
Mutations Producing Favourable Metrics
for FcγRIIb Selectivity in silico

| | | Mutations | |
|---|---|---|---|
| Region of Fc | Position | Chain A | Chain B |
| Hinge | G236 | G236K | G236D; G236N; G236L |
| Hinge | G237 | | G237I; G237E; G237Q; G237M; |
| | | | G237H; G237W |
| S1 | S239 | S239W | |
| L3 | P329 | | P329K; P329W; P329H; P329F; |
| | | | P329Y |

### 3) Mutations Based on IgG4

Reported binding affinities of IgG1 and IgG4 for the Fcγ receptors show a measurable selectivity of IgG4 towards FcγRIIb (see Table 1.4 below).

TABLE 1.4
K_{D} Values for IgG1 and IgG4 Binding to Human Fcγ Receptors*

| | FcγRI | FcγRIIaH | FcγRIIaR | FcγRIIb | FcγRIIc | FcγRIIIaV | FcγRIIIaF |
|---|---|---|---|---|---|---|---|
| IgG1 | 1.50E−08 | 1.90E−07 | 2.90E−07 | 8.30E−06 | 8.30E−06 | 5.00E−07 | 8.50E−07 |
| IgG4 | 2.90E−08 | 5.90E−06 | 4.80E−06 | 5.00E−06 | 5.00E−06 | 4.00E−06 | 5.00E−06 |
| Kₐ(IgG4)/ | 0.52 | 0.03 | 0.06 | 1.66 | 1.66 | 0.13 | 0.17 |
| Kₐ(IgG1) | | | | | | | |

*Moldt and Hessell, Antibody Fc, Ch. 8: FcγRs Across Species, 2013, Elsevier Inc.

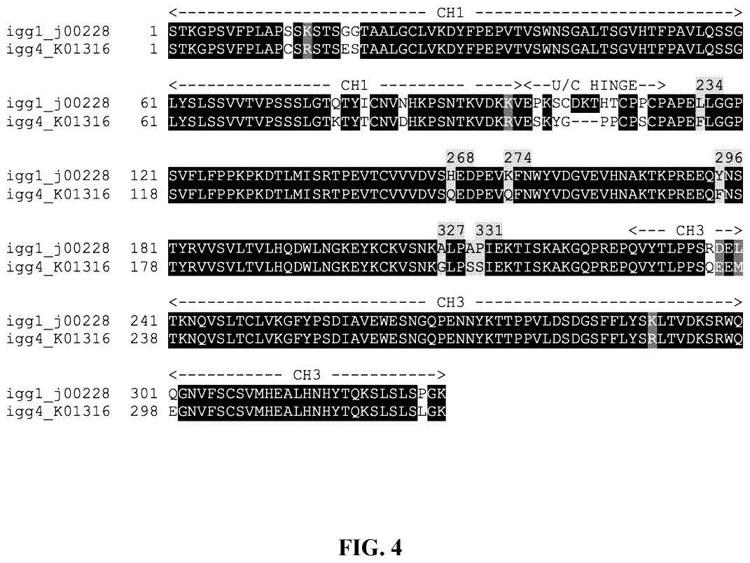

Sequence alignment of IgG1 and IgG4 shows a number of differences in the lower hinge and CH2 regions (see FIG. 4).

Based on the above, the following mutations and combinations of mutations were selected to investigate the selectivity of IgG4 towards FcγRIIb:
1. Loop 3 mutations: A327G, A330S, P331S
2. Hinge mutation: L234F
3. Loop 1 mutations: H268Q, Q274K
4. Loop 3 mutations+Loop 1 mutations
5. Loop 3 mutations+Loop 1 mutations+Hinge mutation
6. Loop 3 mutations+Loop 1 mutations+Hinge mutation+Loop 2 mutations (F296Y)

### 1.3 Deconvolution of Asymmetric Binding

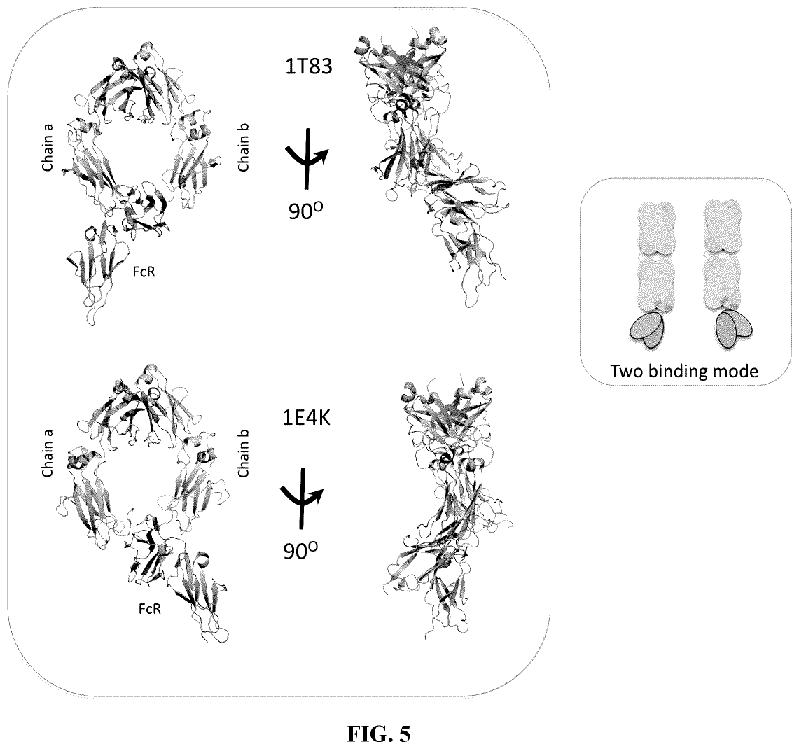

The symmetry of the homodimeric Fc antibody and the structures of the Fc/FcγR complex reveal the presence of at least two binding modes for the Fc to the receptor (see FIG. 5). In asymmetric design, the effect of the asymmetric mutations should be evaluated for both binding modes: the designed variant and its mirror variant. In silico data has shown that the negative design of asymmetric variants that disrupts binding to specific receptors often does not have the same effect in the mirror variant. Hence, the specificity gained by the asymmetric mutations can be lost if the second binding mode is still allowed.

The mutation E269K in the CH2 domain of the Fc region is known to abrogate binding to the Fcγ receptors when introduced symmetrically in both chains of the CH2 domain. If this mutation is introduced asymmetrically on only one of the two chains of the CH2 domain, then the mutation acts as a “Polarity Driver” by blocking binding of the FcγR at the face where the mutation is present, while letting the other face of the Fc interact with the FcγR in a normal manner.

Each of the selected variants was tested with the E269K Polarity Driver (PD) in order to deconvolute the binding of the variant to FcγRIIb and determine whether the mutation was effective in Chain A or Chain B of the Fc. A total of three constructs per mutation was required as shown in Table 1.5, where X=mutation being evaluated, and PD=Polarity Driver.

TABLE 1.5
Constructs for Deconvolution

| Construct | Chain A | Chain B | Comment |
|---|---|---|---|
| 1 | | X + PD | Test mutation and PD in same chain |
| 2 | PD | X | Test mutation and PD in opposite chains |
| 3 | X | X | Homodimeric control |
| 4 | | PD | Control PD alone (same for all variants |
| | | | tested) |

The wild-type P329 residue was identified in Example 1.2, part 1), as being a hot spot mutation. As such, mutations at position P329 were tested in the presence of binding enhancers as well as the PD. The mutations H268D and S267E had been shown to be binding enhancers for FcγRIIb, and combination of these two mutations resulted in 100-fold improvement in binding. As such, these two mutations were used as binding enhancers when testing the P329 mutations. The PD is expected to reduce this 100-fold improvement in binding down to 50-fold. The P329 mutations were therefore evaluated for their ability to reduce binding to FcγRIIaR/FcγRIIaH to below wild-type levels while reducing binding to FcγRIIb to approximately wild-type levels in the presence of the binding enhancers and the PD. The constructs tested for the P329 mutations are shown in Table 1.6.

TABLE 1.6
Constructs Containing P329 Mutations

| Construct | Chain A | Chain B | Comments |
|---|---|---|---|
| 1 | S267E/H268D | E269K/P329K | Selective P329 driver + PD |
| 2 | S267E/H268D | P329K | Selective P329 driver |
| 3 | S267E/H268D | E269K/P329W | Selective P329 driver + PD |
| 4 | S267E/H268D | P329W | Selective P329 driver |
| 5 | S267E/H268D | E269K/P329H | Selective P329 driver + PD |
| 6 | S267E/H268D | P329H | Selective P329 driver |
| 7 | S267E/H268D | E269K/P329F | Selective P329 driver + PD |
| 8 | S267E/H268D | P329F | Selective P329 driver |
| 9 | S267E/H268D | E269K/P329Y | Selective P329 driver + PD |
| 10 | S267E/H268D | P329Y | Selective P329 driver |

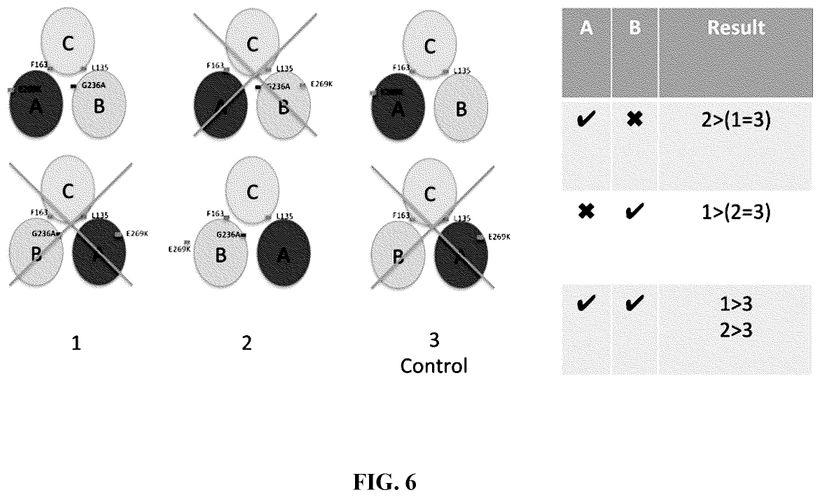

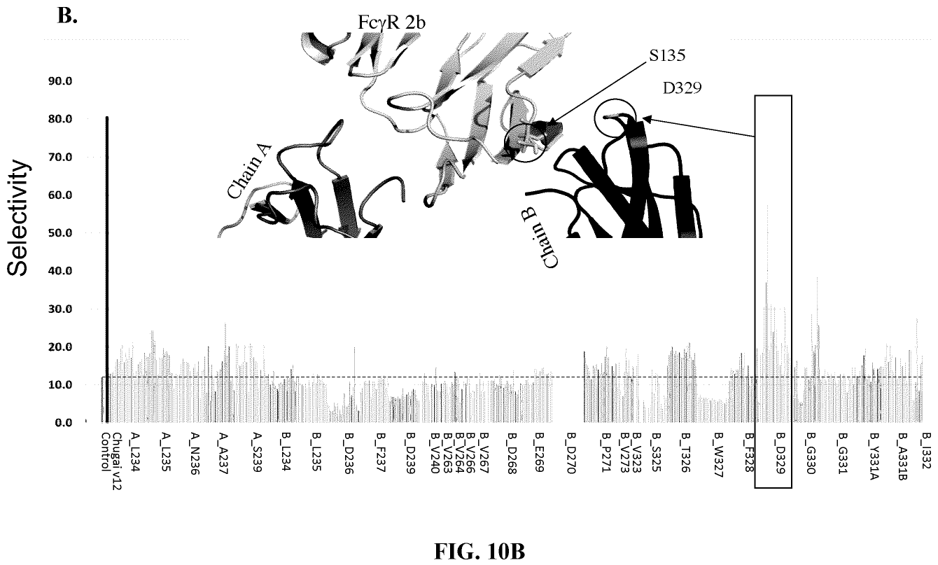

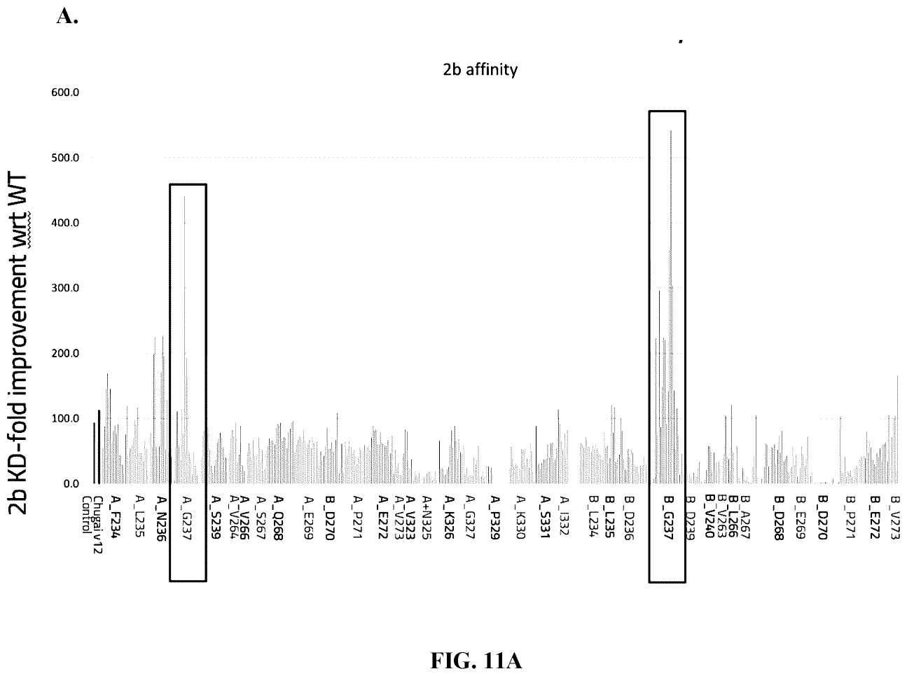

The contribution of a given mutation to FcγR binding in each chain was determined as described below with reference to FIG. 6. Three constructs were used to deconvolute the contribution of a given mutation. In FIG. 6, the mutation G236A is used as an exemplary mutation. G236A has shown increased binding to the FcγRIIb receptor, but it was unclear how the mutation was driving the selectivity. In all constructs shown in FIG. 6, E269K is used as a polarity driver, which blocks binding to the FcγR only in the binding mode in which it is most proximal to position L135 (and R134) in the receptor. This binding mode is marked with a cross in FIG. 6. The nomenclature for Chain A and Chain B used below and in FIG. 6 is based on the structure of the human IgG1 Fc/FcγRIII complex available under the Protein Data Bank (PDB) ID 1E4K (see FIG. 10, Chain A is characterized by hotspot P329, and chain B is characterized by hotspot D270).

In construct 1 of FIG. 6, the G236A mutation is in a different heavy chain to the PD (E269K), so only the binding mode in which G236A is close to the L135 residue of the receptor is allowed as shown in the top structure. In construct 2, the G236A mutation is on the same heavy chain as the PD, so only the binding mode where G236A is more proximal to the F163 residue of the receptor is allowed as shown in the lower structure. In construct 3, the PD is tested alone and binding is allowed only when the PD is more proximal to the F163 residue of the receptor.

By comparing the binding of the 3 constructs, it is possible to deconvolute the contribution of the G236A mutation. If it is a “Chain A” driving mutation, then construct 2 would show higher binding than construct 3, which should be similar to construct 1. If it is a “Chain B” driving mutation, then construct 1 would show higher binding than constructs 3 and 2. If it is a mutation important for both chains, then both constructs 1 and 2 would show better binding than construct 3. This analysis assumes additive contributions that are independent of each other. In the case of a synergistic contribution, both constructs 1 and 2 would show same binding as construct 3, but a symmetric construct would be better than all the other constructs. The various possible outcomes described above are summarized in Table 1.7.

TABLE 1.7
Deconvolution of Asymmetric Mutation
X using Polarity Driver (PD)

| Variant | | | | |
|---|---|---|---|---|
| Mutation X | Mutation X | | Mutation X | |
| in Same Chain | in Opposite | PD | on both chains, | |
| as PD | Chain to PD | alone | no PD | Assessment |
| ++ | + | + | ++++ | Chain A |
| | | | | mutation |
| + | ++ | + | ++++ | Chain B |
| | | | | mutation |
| ++ | ++ | + | ++++ | Chain AB |
| | | | | additive |
| | | | | mutation |
| + | + | + | ++++ | Chain AB |
| | | | | synergistic |
| | | | | mutation |
| ++ | + | + | + | Chain A |
| | | | | mutation, AB |
| | | | | is detrimental |
| + | ++ | + | + | Chain B |
| | | | | mutation, AB |
| | | | | is detrimental |

Variants comprising the asymmetric mutations were constructed in a one-armed antibody scaffold (Scaffold 2) and tested for FcγR binding by SPR as described in the General Methods (Protocol 1). Thermal stability of the variants was also tested by DSF as described in the General Methods.

The most selective variants identified by the above approaches are shown in Table 1.8. The results for the deconvolution of the mutations comprised by these variants are shown in Table 1.9.

TABLE 1.8
Most Selective Variants Identified in Initial Screen

| | | | | K_{a, mut}/ | K_{a, mut}/ | |
|---|---|---|---|---|---|---|
| | Chain A | Chain B | ΔTm/ | K_{a, WT} | K_{a, WT} | Selectivity |
| Category¹ | Mutation(s) | Mutation(s) | ° C³ | FcγRIIb | FcγRIIaR | IIb/IIaR |
| A1X | G236N | G236N | 0 | 0.4 | 0.08 | 5 |
| IgG1/IgG4 | A327G_A330S_{—} | A327G_A330S_{—} | −3.5 | 2.03 | 0.47 | 4.4 |
| | P331S_L234F_{—} | P331S_L234F_{—} | | | | |
| | H268Q_K274Q | H268Q_K274Q | | | | |
| S1X | S267L | S267L | −2 | 0.98 | 0.33 | 3 |
| S1X | S267I | S267I | −4 | 2.69 | 0.98 | 2.8 |
| A1X | G236D | G236D | −2 | 2.22 | 0.81 | 2.7 |
| S1X | L234D | L234D | −1 | 0.47 | 0.17 | 2.7 |
| IgG1/IgG4 | L234F | L234F | 0 | 0.52 | 0.21 | 2.5 |
| IgG1/IgG4 | L235D | H268Q_K274Q | −7.5 | 2.24 | 1.03 | 2.2 |

¹A1X—asymmetric 1× screen; S1X—symmetric 1× screen
² Compared to wild-type

TABLE 1.9
Deconvolution of Mutations from Most Selective Variants

| | | Chain A | Chain B | IIb- | IIaR- | IIb/IIaR | |
|---|---|---|---|---|---|---|---|
| Variant # | Category¹ | Mutations | Mutations | Fold² | Fold² | Selectivity | Comments |
| 16488 | A1X | | E269K_{—} | 0.16 | 0.17 | 0.91 | Chain B |
| | | | G236D | | | | |
| 16489 | A1X | E269K | G236D | 0.85 | 0.41 | 2.10 | |
| 16490 | A1X | G236D | G236D_{—} | 2.22 | 0.81 | 2.75 | |
| 16491 | A1X | | E269K_{—} | 0.24 | 0.10 | 2.48 | Chain A |
| | | | G236N | | | | |
| 16492 | A1X | E269K | G236N | 0.13 | 0.07 | 1.83 | |
| 16493 | A1X | G236N | G236N | 0.40 | 0.08 | 4.99 | |
| 16531 | S1X | | E269K_{—} | 0.27 | 0.19 | 1.45 | Chain B |
| | | | S267I | | | | |
| 16532 | S1X | E269K | S267I | 0.95 | 0.51 | 1.84 | |
| 16533 | S1X | S267I | S267I | 2.69 | 0.98 | 2.75 | |
| 16549 | S1X | | E269K_{—} | 0.35 | 0.19 | 1.83 | Chain A |
| | | | L234D | | | | |
| 16550 | S1X | E269K | L234D | 0.79 | 0.63 | 1.25 | |
| 16585 | S1X | L234D | L234D | 0.47 | 0.17 | 2.70 | |
| 16570 | IgG1−>IgG4 | | E269K_{—} | 0.37 | 0.11 | 3.35 | Chain A |
| | | | L234F | | | | |
| 16571 | IgG1−>IgG4 | E269K | L234F | 0.40 | 0.42 | 0.97 | |
| 16572 | IgG1−>IgG4 | L234F | L234F | 0.52 | 0.21 | 2.48 | |
| 16576 | IgG1−>IgG4 | | E269K_{—} | 0.21 | 0.11 | 1.89 | Chain B for |
| | | | A327G_{—} | | | | affinity, |
| | | | A330S_{—} | | | | Chain A for |
| | | | P331S_{—} | | | | selectivity |
| | | | L234F_{—} | | | | |
| | | | H268Q_{—} | | | | |
| | | | K274Q | | | | |
| 16577 | IgG1−>IgG4 | E269K | A327G_{—} | 1.03 | 0.82 | 1.26 | |
| | | | A330S_{—} | | | | |
| | | | P331S_{—} | | | | |
| | | | L234F_{—} | | | | |
| | | | H268Q_{—} | | | | |
| | | | K274Q | | | | |
| 16578 | IgG1−>IgG4 | A327G_{—} | A327G_{—} | 2.03 | 0.47 | 4.35 | |
| | | A330S_{—} | A330S_{—} | | | | |
| | | P331S_{—} | P331S_{—} | | | | |
| | | L234F_{—} | L234F_{—} | | | | |
| | | H268Q_{—} | H268Q_{—} | | | | |
| | | K274Q | K274Q | | | | |

¹A1X—asymmetrical 1X screen; S1X—symmetrical 1X screen
²Fold affinity over wild-type

### Example 2: Loop Replacements

The L3 (FG) loop in the CH2 domain of chain B of the IgG Fc (referred to herein as the “B/325 loop”) is not directly involved in FcγR binding (see FIG. 2B) and makes negligible contributions to the CH2 domain stability. Consequently, this loop is attractive target for engineering FcγRIIb selectivity. An analysis of the available crystal structures and implicit solvent MD simulations performed on the wild-type Fc/FcγR complex showed that the residues comprised by the B/325 loop are typically distant from position 135 on the FcγR (the “C/135 site”). The typical minimum C_{β}-C_{β} distance between the target C/135 site and the closest residue on the B/325 loop was determined to be approximately 10×. Based on this structural analysis, the B/325 loop was engineered in order to extend the loop such that it interacted directly with the receptor near site C/135, and in this way drive selective binding to FcγRIIb. Engineering of the B/325 loop proceeded following the steps detailed below.

### 2.1 Template Search

The B/325 loop in the Fc can be extended either by inserting residues into the wild-type (WT) loop sequence or by replacing the WT loop with an entirely new loop or a combination of a loop and secondary structure. The approach taken here was to replace the entire L3 loop (positions 325-331) in the WT Fc with a novel protein insert or “template.” A “template” is a polypeptide segment sourced from an existing protein structure available in the Protein Data Bank (PDB). The nomenclature used to refer to the various parts of such a template is shown in FIG. 7.

The initial template identification process was intended to identify protein components that, given their wild-type sequence, would have a conformation that placed part of the template backbone close to receptor site C/135 when the Fc was bound. Selectivity enhancing mutations could then be added to the templates as described below to drive selective binding to the FcγRIIb receptor. The initial template search phase was designed to identify templates that would produce an Fc with the following properties:
1. Adequate protein expression
2. Sufficient stability to enable experimental evaluation
3. Demonstrated ability to alter Fc/FcγR binding affinity without completely abrogating binding.

To find such templates, the following criteria were used when searching the PDB:
1. Crystal structures with a resolution better than 2.5 Å
2. Templates consisting of a loop anchored by β-strands
3. Backbone heavy atom RMSD of the template anchors to residues B/324 and B/332≤0:85 Å
4. Total length of template=7-12 amino acids
5. Template includes at least one β-stranded residue at both the N- and C-termini of the template
6. Template includes at least one hydrogen bond between β-stranded residues located at opposite termini of the template
7. β-stranded residues at the C-terminus of the template do not form hydrogen bonds with any residue in the source structure other than residues found at the N-terminus of the template.

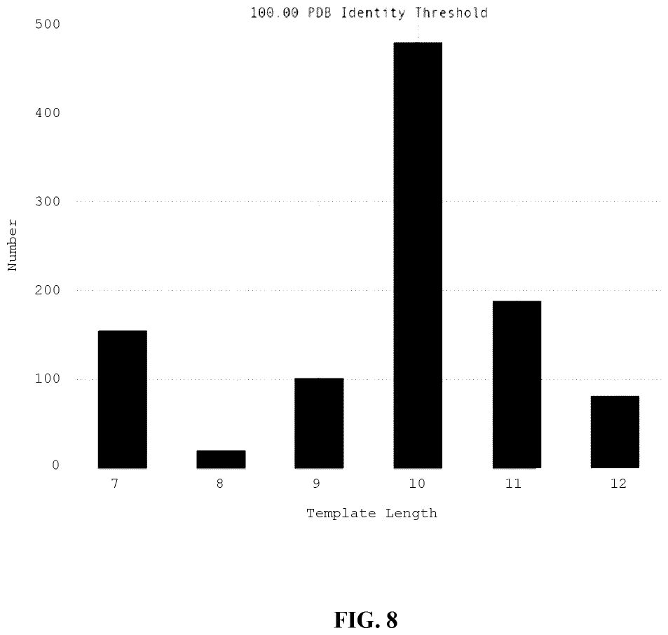

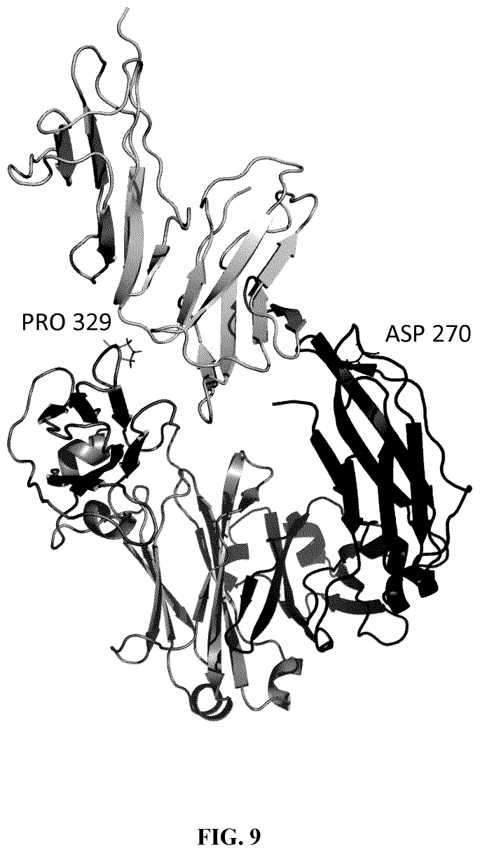

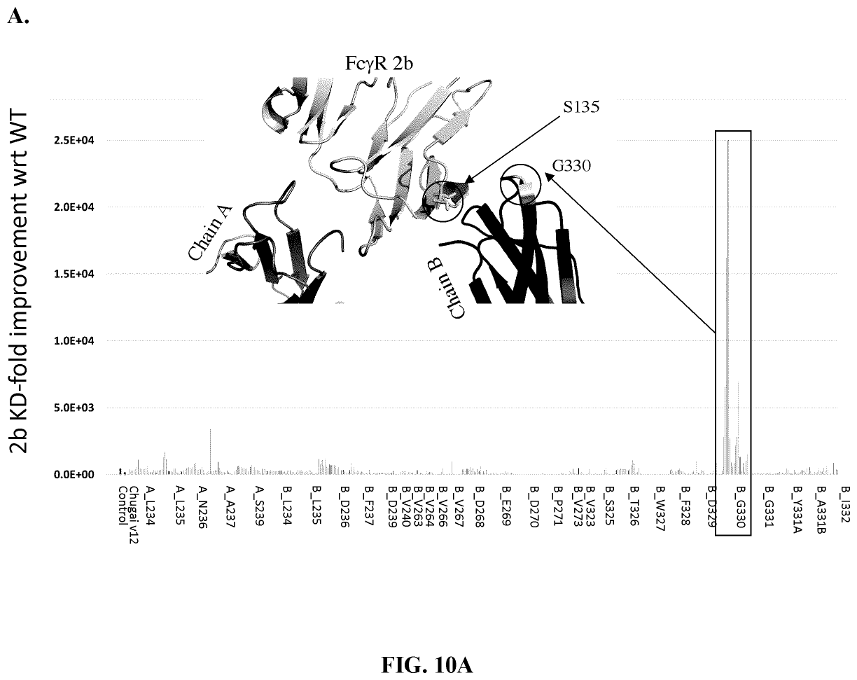

When compiling a list of templates meeting these criteria, STRIDE (Frishman & Argos, 1995, Proteins Struct. Funct. Bioinf, 23:566-579) was used to assign secondary structure classifications to the residues in the PDB structures included in the search. Running a search with these criteria against the representative structures in the 100% clustering generated by the PDB (Yang, et al., 2018, Nucleic Acids Res., 47:D464) yielded a total of 1026 templates with the length distribution shown in FIG. 8.

2.2 Grafting Templates into the Fc

All 1026 templates identified in the initial PDB search were grafted in silico into the Fc/FcγRIIb complex structure using the following steps:
1. Residues B/325-B/331 inclusive were deleted from the Fc/FcγRIIb complex.
2. The template backbone was introduced into the Fc/FcγRIIb complex by aligning the backbone heavy atoms of the template anchors to residues B/324 and B/332 of the Fc/FcγRIIb complex.
3. The coordinates of the backbone atoms for residues B/323, B/324, B/332, B/333 and the first two residues and the last two residues of the template were minimized using the AMBER99SB force field (Hornak, et al., 2006, Proteins Struc. Funct. Bioinf, 65:712) and a conjugate gradient minimizer.

### 2.3 Initial Template Selection

Given the large number of templates found using the approach described above, additional filters were required to isolate a smaller set for further analysis. The following coarse contact potential was, therefore, developed:

$$c ( {\vec{r}}_{i} , {\vec{r}}_{j} ) = \{ \begin{matrix}1 & if {d}_{ij} < ‖ {\vec{r}}_{i} - {\vec{r}}_{j} \leq α ( i , j ) \\ 0 & otherwise\end{matrix}$$

where dᵢⱼ is the sum of the van der Waals radii for atoms i and j.

The empirical upper bound on the contact distance between two atoms was defined as:

$$α ( i , j ) = \{ \begin{matrix}9 A & if atoms i , j are both {C}_{β} atoms \\ 7.5 A & if one of atoms i , j is a {C}_{β} atoms \\ 6 A & otherwise\end{matrix}$$

In this application, c(i;j) was only computed between C_{β} and backbone heavy atoms of residues comprised by the template, and the C_{β} and backbone heavy atoms of residue C/135 on the FcγR. When performing the preliminary evaluation of templates, it was important to determine if the grafted configuration of the template had a length and orientation that could allow one or more template residues to interact with the FcγR at or near site C/135. A template with a high value of c(i;j) summed over all template backbone and C_{β} atoms would be in a position that could facilitate these direct interactions. The use of this coarse contact filter provided a simple first pass screening method for reducing the set of potential templates. A minimum coarse contact count of six was set for filtering templates, which corresponds to the value of the top quartile of the length nine templates. As a reference, the B/265 loop in the IgG Fc has 36 coarse contacts and the B/298 loop forms 44 contacts. Both these loops are known to play a critical role in Fc/FcγR binding and, as such, a minimum threshold of six coarse contacts was expected to be permissive. Applying this filter reduced the number of templates to 285.

### 2.4 Structure Optimization

All templates that passed the coarse contact filter underwent side-chain repacking with backbone relaxation. The side-chain repacking procedure employed a variant of the ICM algorithm with a fine-grained rotamer library (Xiang & Honig, 2001, J. Mol. Biol., 311:421). The backbone coordinates were relaxed via 5000 steps of the backrub algorithm (Betancourt, 2005, J Chem. Phys., 123:174905; Smith & Kortemme, 2008, J. Mol. Biol., 380:742). All refinement was performed using the AMBER99SB force-field (Hornak, et al., 2006, Proteins Struc. Funct. Bioinf, 65:712), the GB/OBC implicit solvent model (Onufriev, et al., 2004, Proteins Struc. Funct. Bioinf, 55:383), and a pairwise hydrophobic potential (Jacobsen, et al., 2004, Proteins Struc. Funct. Bioinf, 55:351). When repacking, the sequence of the template was taken to be the wild-type sequence of the template residues as found in the PDB structure from which the template was extracted. After repacking and backbone optimization, the structures were checked for inter-atomic clashes. Atoms i and j were considered to be clashing when σᵢ+σⱼ−dᵢⱼ>0:4. Here, σᵢ is the van der Waals radius of atom i as defined in the AMBER99SB force field, and dᵢⱼ is the distance between atoms i and j. Templates that had clashes after repacking were eliminated from further consideration.

### 2.5 Secondary Template Selection

After repacking, all templates were re-evaluated using the coarse contact score, and the minimum C_{β}-C_{β} distance between any residue on the template and the C_{β} atom on receptor residue C/135 was also computed. The Pareto optimal (Li, et al., 2010, BMC Struc. Biol., 10:22) templates were then identified on the basis of anchor backbone heavy atom RMSD, coarse contact score and minimum C_{β}-C_{β} distance.

Templates on the first three Pareto optimal fronts were identified and pairwise sequence similarities were then computed for all templates of a common length in the optimal set. There was considerable sequence diversity in the optimal set, with a maximum within-set sequence identity of 0.9 occurring for a single pair of templates. The mean maximum-pairwise sequence identity within the optimal set was 0.44.

### 2.6 Template Perturbation

Given that the templates were sourced from extant PDB structures with native environments very different from that experienced in the Fc/FcγR complex, it was assumed that most of the templates would change conformation in the new environment. Consequently, the stability of the template conformations in the Fc/FcγRIIb complex was tested using a simple molecular dynamics (MD)-based simulated annealing approach.

In the first step of this approach, a mobile region was defined by placing an arginine residue at each site on the template, rotating the residue through every rotamer in the Dunbrack rotamer library (Dunbrack & Karplus, 1993, J. Mol. Biol., 230:543) and enumerating all Fc/FcγR residues with a heavy atom less than 4.0 Å from a heavy atom of the test arginine in any rotameric configuration. The union of all residues identified in this manner resulted in a “mobile zone.” No restraints or constraints of any type were placed on residues in this zone. All residues not included in the mobile zone were held fixed.

With this mobile zone defined, each template was run through a simulated annealing protocol. The annealing simulations were performed using the OpenMM molecular dynamics package (Eastman, et al., 2013, J. Chem. Theory Comput., 9:461), the AMBER99SB force-field, and the GB/OBC implicit solvation model. The protocol included the following steps:
1. A short (2 ns) high-temperature simulation was performed at 500K. The simulation started from the repacked structures produced using the protocol described previously.
2. The conformations from the second half of the trajectory produced in step 1 were clustered into ten clusters using the k-means algorithm.
3. Starting from the conformations identified in step 2, ten separate annealing simulations were performed. The temperature schedule consisted of cooling geometrically from 500K to 450K over 1.0 ns, followed by a linear cooling stage from 450K to 300K over 19 ns. No restarts were performed.
4. The low temperature components (300K-302K) of each of the ten annealing trajectories were extracted and used for subsequent analysis. Combined, the ten annealing runs generated 3 ns of trajectory data for each template.

### 2.7 Final Template Selection

The aggregate trajectory produced in step 4 of the annealing procedure was clustered using the SPICKER clustering method (Zhang & Skolnick, 2004, J Comput. Chem., 25:865). Clustering was performed on the backbone heavy atoms of the template. As the majority of the Fc/FcγR structure was held fixed during the annealing simulations, the variations in the conformations of templates had contributions both from internal deformation of the template, and relaxation of the anchoring β-strands. Only the primary cluster returned by the SPICKER algorithm was considered in further analysis.

By construction, the primary clusters contained between 60% and 70% of the total frames in the aggregate trajectory produced in step 4 of the annealing procedure. Using the primary clusters, the following quantities were computed:
1. The mean number of coarse contacts between the template and site C/135 on the FcγRIIb receptor.
2. The RMSF of the template (computed on the basis of the template backbone heavy atoms).
3. The mean backbone heavy atom RMSD (computed relative to the grafted structure of the template).

The coarse contact score indicated if the low-temperature structures generated by the annealing processes had configurations that were in position to interact with C/135. The RMSF served as a measure of consistency between and within the annealing runs. Templates with low RMSF values showed consistency in structure across the annealing runs, indicating that the runs were well converged. Low RMSF values also indicated that the template was not overly flexible and, as such, templates with low RMSF were favored in subsequent selection rounds. Finally, a low backbone RMSD to the grafted structure indicated that the template did not significantly deviate from the wildtype conformation found in the PDB from which the template was derived. Templates which showed low backbone RMSDs to the grafted conformation were also favored.

This set of metrics was computed for each of the templates from the secondary template selection and used to select a set of templates for experimental screening. The criteria used to select templates was a coarse contact count ≥5, and either a reference RMSD or an RMSF less than 3.0 Å. Ten templates were selected using these criteria. Two other templates were selected on the basis of a visual review of the cluster centroid produced by the SPICKER clustering method.

### 2.8 Alternate Templates

After generation of the initial set of templates as described above, a second template search phase was conducted. This second template search followed the same protocol as the first search with the following modifications:
1. All templates selected in the first search were excluded.
2. The hydrogen bonding filter was not employed.
3. The maximum temperature in the annealing process was reduced from 500K to 325K.

A second set of ten templates for experimental screening selected through this search.

### 2.9 Initial Experimental Screening

Based on the in silico screening methods described above, as well as two other in silico screening rounds using similar selection criteria, the loop templates shown in Table 2.1 were selected for experimental testing.

TABLE 2.1
Loop Templates Selected for Experimental Testing

| | | Source PDB | | | | | |
|---|---|---|---|---|---|---|---|
| Template | Sequence | ID | Start | | | Coarse | AMBER |
| ID | [SEQ ID NO] | (Organism) | Residue ID | RMSD_{Ref}¹ | RMSF | Contacts | Affinity¹ |
| 231 | WTDQSGQDR | 1QVC | A/88.TRP | 1.81 +/− | 0.73 | 4 | −2.05 |
| | [SEQ ID NO: 4] | (Bacteria) | | 0.21 | | | |
| 168 | LDMEGRKIH | ILN1 | A/123.LEU | 0.87 +/− | 0.33 | 5 | 0 |
| | [SEQ ID NO: 5] | (Human) | | 0.09 | | | |
| 356 | HIDNQGYENL | 3A15 | A/84.HIS | 0.68 +/− | 0.41 | <1 | −0.38 |
| | [SEQ ID NO: 173] | (Bacteria) | | 0.13 | | | |
| 255 | VDINGKKVK | 2EQB | A/62.VAL | 0.83 +/− | 1.03 | 1 | 9.14 |
| | [SEQ ID NO: 174] | (Yeast) | | 0.07 | | | |
| 53 | YVSFNGATDE | 3CIN | A/298.TYR | 1.05 +/− | 0.54 | 10 | −6.31 |
| | [SEQ ID NO: 175] | (Bacteria) | | 0.20 | | | |
| 1 | STWFDGGYAT | 2GKO | A/235.SER | 1.94 +/− | 1.24 | 3 | −10.2 |
| | [SEQ ID NO: 6] | (Bacteria) | | 0.42 | | | |
| 11 | HFDENGEIVT | 2DWC | A/218.HIS | 0.77 +/− | 0.6 | 3 | −16 |
| | [SEQ ID NO: 7] | (Archaea) | | 0.22 | | | |
| 47 | GIAYDGNLLK | 1I4U | A/69.GLY | 1.14 +/− | 0.65 | 10 | 0.72 |
| | [SEQ ID NO: 176] | (Lobster) | | 0.28 | | | |
| 5 | FQDTSGNVFY | 2W3Y | A/178.PHE | 0.69 +/− | 0.55 | 2 | 10.58 |
| | [SEQ ID NO: 177] | (Bacteria) | | 0.15 | | | |
| 48 | ITLQDQRRVW | 2HLC | A/35.ILE | 0.54 +/− | 0.45 | 3 | 10.4 |
| | [SEQ ID NO: 178] | (Insect) | | 0.14 | | | |
| 28 | VEFEDGDRRL | 1JJ2 | A/58.VAL | 0.57 +/− | 0.38 | 12 | 7.51 |
| | [SEQ ID NO: 179] | (Archaea) | | 0.10 | | | |
| 38 | LIDENGNEQK | 3GVE | A/150.LEU | 0.81 +/− | 0.38 | 13 | −6.31 |
| | [SEQ ID NO: 10] | (Bacteria) | | 0.12 | | | |
| 82 | YTDSEDGATNI | 3LYV | A/33.TYR | 0.61 +/− | 0.39 | 12 | −24.53 |
| | [SEQ ID NO: 180] | (Pathogenic | | 0.10 | | | |
| | | Bacteria) | | | | | |
| 7 | GLDEEGKGAV | 4R30 | M/112.GLY | 0.52 +/− | 0.43 | 16 | −36.82 |
| | [SEQ ID NO: 8] | (Human) | | 0.12 | | | |
| 19 | VTWEDGKSER | 1OID | A/323.VAL | 0.90 +/− | 0.43 | 20 | −8.24 |
| | [SEQ ID NO: 9] | (Bacteria) | | 0.10 | | | |
| 66 | DFDQNQGEVV | 1IJR | A/47.ASP | 0.84 +/− | 0.53 | 23 | −7.06 |
| | [SEQ ID NO: 12] | (Human) | | 0.18 | | | |
| 3 | GIDLSTGLPRK | 1JCF | A/228.GLY | 0.91 +/− | 0.64 | 17 | −6.64 |
| | [SEQ ID NO: 181] | (Bacteria) | | 0.15 | | | |
| 60 | VQDATGAPFL | 3E35 | A/99.VAL | 1.05 +/− | 0.48 | 12 | −3.19 |
| | [SEQ ID NO: 11] | (Bacteria) | | 0.18 | | | |
| 151 | LTDEEGRPYR | 4JN3 | A/67.LEU | 0.84 +/− | 0.54 | 12 | −21.52 |
| | [SEQ ID NO: 14] | (Bacteria) | | 0.23 | | | |
| 83 | SDFEGKPTL | 2X6C | A/151.SER | 0.88 +/− | 0.43 | 12 | −7.12 |
| | [SEQ ID NO: 13] | (Bacteria) | | 0.20 | | | |

¹Averaged over the dominant cluster (obtained using SPIKER clustering)

Variants in which residues 325-331 in chain B of the Fc were replaced with one of the selected loop templates were constructed in a one-armed antibody scaffold (Scaffold 2) and tested for FcγR binding by SPR as described in the General Methods (Protocol 1). Thermal stability of the variants was also tested by DSC as described in the General Methods (Protocol 1). The templates shown in Table 2.2 gave the best results and were selected for further testing.

TABLE 2.2
FcγR Binding for Variants Comprising Top Loop Templates

| Template | | SEQ ID | IIb- | IIb/IaR | DSC |
|---|---|---|---|---|---|
| ID | Sequence | NO | Fold¹ | Selectivity | ΔTm/°C.² |
| 231 | WTDQSGQDR | 4 | 5.3 | 0.88 | −6.2 |
| 168 | LDMEGRKIH | 5 | 2.9 | 0.9 | 2.3 |
| 1 | STWFDGGYAT | 6 | 3.7 | 1.8 | −3.0 |
| 11 | HFDENGEIVT | 7 | 3.0 | 1.5 | 0 |
| 7 | GLDEEGKGAV | 8 | 4.9 | 1.8 | −4.0 |
| 19 | VTWEDGKSER | 9 | 0.8 | 1.6 | 0 |
| 38 | LIDENGNEQK | 10 | 4.3 | 1.5 | 1.8 |
| 60 | VQDATGAPFL | 11 | 7.7 | 1.7 | 0.3 |
| 66 | DFDQNQGEVV | 12 | 8.1 | 1.6 | −7.6 |
| 83 | SDFEGKPTL | 13 | 3.4 | 1.6 | −5.2 |
| 151 | LTDEEGRPYR | 14 | 8.5 | 1.7 | 0 |

¹Fold change in affinity over wild-type
²Compared to wild-type

### 2.10 Engineering Selectivity-Enhancing Mutations

The templates identified in Table 2.1 showed enhanced, but non-selective binding affinity to FcγRIIa and FcγRIIb. The ability to positively modulate binding affinity, combined with the structural analysis performed during template selection, strongly suggested that a number of these templates had a conformation that would place part of the template near the FcγR C/135 site. Accordingly, the next step was to introduce mutations that could drive FcγRIIb binding selectivity.

As the loop templates replaced residues 325-331 in the parental Fc sequence, the following numbering system is used for the loop templates in the discussion below and the following Examples. The residue immediately following position 324 in the Fc is designated 325*, the remaining residues of the loop template are numbered sequentially from 326* to 331*. Any additional residues after 331* in the loop template are designated a letter, i.e. 331*A, 331*B, 331*C, etc.

In silico analysis of the relative positions of the template loops inserted into the Fc and the C/135 site of the FcγR indicated that positions 327*-329* of the loop are best positioned to interact with C/135 in the receptor.

To identify residues that could be introduced at one of positions 327*-329* to potentially differentiate between S135 in FcγRIIb and L135 in FcγRIIa, the PDB was searched to identify the probability of finding each of the 20 amino acids within a reasonable distance of Ser and Leu residues. The results indicated that Asp, Asn, Ser, Glu, His and Gly are more commonly found in proximity to Ser residues than Leu residues. In contrast, Ile, Leu, Met, Val and Phe are more commonly found near Leu residues than Ser residues. These results are consistent with the expectation that polar and charged residues capable of hydrogen bonding will be enriched in the vicinity of Ser residues, while the regions near Leu residues will be dominated by hydrophobic residues.

Based on the above analysis, the residues ASP, ASN, SER, GLU, HIS and GLY, as well as THR and GLN, were selected for testing in combinatorial fashion in the top loop templates. Additionally, as some PDB structural homologues to the selected loop templates showed the presence of PRO, which is potentially important for loop stability and folding, PRO was also included in the combinatorial screen.

In addition, mutations at positions that could potentially affect the conformation of the loop were tested. In particular, positions 325*, 327*, 331*A, 331*1 and 332 were identified as positions that could potentially affect the conformation of the loop and mutations at these positions were tested in a FX scan.

The additional mutations analyzed for their ability to enhance FcγRIIb selectivity of the loop templates are summarized in Tables 2.3 and 2.4.

TABLE 2.3
Loop Template Mutations (Combinatorial) Analyzed
for FcγRIIb Selectivity Enhancement

| Template | Position/Amino Acids Tested | | | | | |
|---|---|---|---|---|---|---|
| ID | 326* | 327* | 328* | 329* | 330* | Other |
| 231 | H | | D, E, T, S, H, N | | | G236W |
| | T(WT) | | Q(WT) | | | |
| 168 | | I | D, E, T, S, H, N | | | G236W |
| | | M(WT) | Q(WT) | | | |
| 1 | H | D | D, E, T, S, H, N, Q, P | G | | |
| | T(WT) | W(WT) | F(WT) | D(WT) | | |
| 11 | | | D, T, S, H, N, Q, P | | | |
| | | | E(WT) | | | |
| 38 | | | D, T, S, H, N, Q | D, E, T, S, H, Q | | |
| | | | E(WT) | N(WT) | | |
| 7 | | | D, T, S, H, N, Q | D, T, S, H, N, Q, R | | |
| | | | E(WT) | E(WT) | | |
| 19 | | | D, T, S, H, N, Q, P | E, T, S, H, N, Q | | |
| | | | E(WT) | D(WT) | | |
| 66 | | N | D, E, T, S, H, N, P | D, E, T, S, H, | D, | |
| | | D(WT) | Q(WT) | N(WT) | Q(WT) | |
| 60 | | | | | | |
| 151 | | | D, T, S, G, N, Q | D, T, S, H, N, Q | | |
| | | | E(WT) | E(WT) | | |
| 83 | | | D, T, S, G, N, Q | | | |
| | | | E(WT) | | | |

TABLE 2.4
Loop Template Mutations (1x Scan) Analyzed for FcγRIIb Selectivity Enhancement

| Template | Position/Amino Acids Tested | | | | | | | |
|---|---|---|---|---|---|---|---|---|
| ID | 325* | 327* | 329* | 330* | 331* | 331*A | 331*B | Other (332) |
| 231 | V, L, T, I, A, | A | | | | I, V, T, L, F, | | |
| | S, H, D, E | | | | | R, S, N, A | | |
| 168 | I, Y, T, V, | E, D, P, L, K, | | S | S | G, Y, S, V, T, | | |
| | K, A | V, R, S, A | | | | A, K, F, R, L | | |
| 1 | T, V, A | Y, G, T, A | | | | | L, N | |
| 11 | V, T, K, F, S, | L, G, A | | | | | L, G, A | |
| | L, I, A | | | | | | | |
| 38 | Y, M, F, N, | A | | | | | I, V, L, R, A, | |
| | K, A | | | | | | Y, K, D, E | |
| 7 | T, V, L, I, | A, T | | | S | | Y, V, T, K, I, | |
| | F, A | | | | | | F, E | |
| 19 | L, A | T, A, S, L, G, | | | S | | L, K, V, R, S, | |
| | | D, Y, N | | | | | A | |
| 66 | N, A, I, V, | A, I, V, L, G, | | | | | N, A, I, L, G, | N, A, V, L, G, S, |
| | L, G, S, T, E, | S, T, E, F, Y, | | | | | S, T, E, F, Y, | T, E, F, Y, H, Q, |
| | F, Y, H, Q, W | H, Q, W | | | | | H, Q, W | W, K, R |
| 60 | Q, S, K, A, | S, Y, K, H, A | D, E, T, S, H, | | | | V, K, T, R, | |
| | | | N, Q | | | | L, A | |
| 151 | N, A, I, V, G, | N, A, I, V, L, | | | S | | N, A, I, V, L, | N, A, V, L, G, S, |
| | S, T, E, F, Y, | G, S, T, E, F, | | | | | G, S, T, E, F, | T, E, F, Y, H, Q, |
| | H, Q, W | Y, H, Q, W | | | | | H, Q, W | W, K, R |
| 83 | K, V, A | D, S, R, A, K, | | S | | V, L, A | | |
| | | E, T, N | | | | | | |

Variants comprising the noted mutations were constructed in a one-armed antibody scaffold (Scaffold 2) and tested for FcγR binding by SPR as described in the General Methods (Protocol 2). The top selectivity variants identified from this screen are shown in Table 2.5.

TABLE 2.5
Top Loop Templates with Selectivity for FcγRIIb

| | | IIb | IIaR | | |
|---|---|---|---|---|---|
| Variant | | Binding | Binding | IIb- | IIb/IIaR |
| # | Description^{1, 3} | KD/M | KD/M | Fold² | Selectivity |
| 16463 | WT | 1.1E−06 | 3.6E−07 | 1 | 1 |
| 18372 | Control without Loop Replacement | 3.9E−08 | 1.7E−08 | 29.3 | 1.4 |
| 20771 | Template_66 | 3.23E−09 | 4.28E−09 | 350 | 4.1 |
| | D327*D_Q328*D_N329*E_Q330*D | | | | |
| 20688 | Template_66 | 3.64E−09 | 4.56E−09 | 311 | 3.9 |
| | D327*D_Q328*P_N329*D_Q330*Q | | | | |
| 20972 | Template_1 | 1.55E−08 | 1.63E−08 | 94.3 | 3.8 |
| | T326*H_W327*W_F328*S_D329*D | | | | |
| 20761 | Template_66 | 3.05E−09 | 3.51E−09 | 372 | 3.6 |
| | D327*D_Q328*E_N329*D_Q330*D | | | | |
| 20976 | Template_1 | 1.58E−08 | 1.58E−08 | 92.5 | 3.6 |
| | T326*H_W327*W_F328*E_D329*D | | | | |
| 20451 | Template_19 | 1.29E−07 | 1.28E−07 | 11.4 | 3.6 |
| | V325*A | | | | |
| 20690 | Template_66 | 8.01E−09 | 3.64E−09 | 141 | 1.4 |
| | D327*D_Q328*Q_N329*N_Q330*Q | | | | |
| 21000 | Template_1 | 1.25E−08 | 1.06E−08 | 117 | 3.0 |
| | T326*T_W327*W_F328*F_D329*D | | | | |
| 20822 | Template_19 | 7.14E−08 | 5.3E−08 | 20.5 | 2.7 |
| | E328*E_D329*D | | | | |

¹Nomenclature used to describe the mutations is based on: Template_Y X327Z, where Y indicates the loop template number, X is the amino acid found at the listed position in the parental loop sequence, and Z is the amino acid mutation.
²Fold change in affinity over wild-type.
³All variants except wild-type included the following mutations in the CH2 domain. Chain A: none; Chain B: S239D_H268D.

### Example 3: Combinations of Mutations

The mutations and loop replacements identified in Examples 1 and 2 that showed the highest selectivity for FcγRIIb or the most significant binding enhancement to FcγRIIb were selected and used to generate combination variants. The selected mutations from Example 1 are summarized in Table 3.1. Combination variants were generated based on these mutations and replacement of residues 325-331 with loop Template 1 (STWFDGGYAT [SEQ ID NO:6]; see Table 2.1).

TABLE 3.1
Summary of Top Mutations

| | | | | IIb/IIaR | IIb/IIaH |
|---|---|---|---|---|---|
| Mutation | IIbY-Fold¹ | IIaH-Fold¹ | IIaR-Fold¹ | Selectivity² | Selectivity² |
| G236K | 0.3 | 0.1 | 0.1 | 4 | 4.4 |
| G236N | 0.3 | 0.2 | 0.1 | 2.5 | 1.3 |
| L234Q | 0.2 | 0.1 | 0.1 | 2.4 | 2.9 |
| L235P | 0.4 | 0.2 | 0.2 | 2.2 | 2.7 |
| L234G | 0.1 | 0 | 0.1 | 2.1 | 2.5 |
| L235S | 0.3 | 0.2 | 0.1 | 2 | 1.5 |
| L235V | 0.5 | 0.4 | 0.2 | 1.9 | 1.1 |
| L234D | 0.4 | 0.1 | 0.2 | 1.9 | 5.5 |
| L235A | 0.4 | 0.2 | 0.2 | 1.9 | 1.7 |
| L234P | 0.3 | 0.2 | 0.2 | 1.8 | 1.6 |
| G236D | 1.5 | 0.6 | 0.8 | 2 | 2.7 |
| S267L | 0.7 | 0.1 | 0.3 | 2.2 | 9.1 |
| L234F | 0.4 | 0.3 | 0.2 | 2 | 1.2 |
| A327G_A330S_P331S_L234F_H268Q_K274Q | 1.3 | 0.3 | 0.5 | 2.8 | 5.3 |
| S267I | 1.9 | 0 | 1.1 | 1.7 | 186 |
| H268D | 4.2 | 2.2 | 4 | 1.1 | 1.9 |
| S239E | 1.4 | 0.6 | 1.4 | 1 | 2.2 |
| S239D | 6.9 | 1.3 | 6.2 | 1.1 | 5.5 |
| V266I | 3.4 | 1.6 | 3.2 | 1.1 | 2.1 |
| V266L | 6.4 | 1.1 | 5.9 | 1.1 | 5.9 |
| S267A | 7.8 | 2.1 | 7.2 | 1.1 | 3.6 |
| Y300E | 3.1 | 0.8 | 2.5 | 1.2 | 3.9 |
| K326D | 6.3 | 1.2 | 6.7 | 0.9 | 5.2 |
| I332E | 3.3 | 1.5 | 2 | 1.7 | 2.3 |

¹Fold affinity change over wild-type as measured by SPR.
²Selectivity is defined as IIbY-Fold/IIaR-Fold or IIbY-Fold/IIaH-Fold

### 3.1 First Group of Combination Variants

For the first group of combination variants, the following strategies were employed in order to select and refine combinations of mutations to improve FcγRIIb selectivity and/or affinity. The number of variants constructed for each strategy is shown in parentheses.
1. Binding Enhancers: Mutations that enhanced FcγR binding in general were combined with mutations that enhance FcγRIIb selectivity but show lower affinity (19 variants).
2. Symmetric variants: 2, 3 or 4 of the selected mutations were combined to provide useful binding profiles (14 variants).
3. IgG4-based variants: Mutations that increase affinity for FcγRIIb were combined with IgG4-based mutations (21 variants).
4. Loop replacements: Mutations to the top loop sequences were made to try to improve the observed enhancement in receptor binding (22 variants).

### 3.2 Second Group of Combination Variants

For the second group of combination variants, the following strategies were employed in order to select and refine combinations of these mutations to improve FcγRIIb selectivity and/or affinity. The number of variants constructed for each strategy is shown in parentheses.
1. Stability engineering: Stabilizing mutations were identified to offset reduction in Tm observed in some variants (31 variants) (see Example 5).
2. Asymmetric variants: Additional mutations were made at positions 234, 235, 236 and/or 237 to try to increase selectivity for FcγRIIb (52 variants).
3. IgG4 variants: Modifications were made to IgG4-based variants to try to increase affinity for FcγRIIb (27 variants).
4. Loop replacements: Modifications were made to loop sequences to try to improve the observed enhancement in receptor binding (51 variants).

### 3.3 Results

Variants from the first and second groups were constructed in a one-armed antibody scaffold (Scaffold 2) and tested for FcγR binding by SPR as described in the General Methods (Protocol 2).

A number of combinations were identified from the first and second groups that improved selectivity and/or affinity of the Fc for FcγRIIb. The top three combination variants are shown in Table 3.2.

TABLE 3.2
Top Combinations of Mutations

| | Variant # | Chain A | Chain B | IIb- | IIaR- | IIb/IIaR |
|---|---|---|---|---|---|---|
| LVG1 | (Approach) | Mutations | Mutations | Fold¹ | Fold¹ | Selectivity² |
| Lead 1 | v19544 | L234D_G236N | Template 1 + | 54.9 | 3.7 | 15 |
| | (Asymmetric | | G236D_S239D_{—} | | | |
| | with loop) | | S267I_H268D | | | |
| Lead 2 | v19585 | L234F_G236N_{—} | L234F_G236D_{—} | 49.5 | 4.1 | 12 |
| | (IgG4-based) | H268Q_K274Q_{—} | S239D_V266L_{—} | | | |
| | | A327G_A330K_{—} | S267A_H268D_{—} | | | |
| | | P331S | K274Q_A327G_{—} | | | |
| | | | A330S_P331S | | | |
| Lead 3 | v19540 | L234D_G236N_{—} | G236D_S239D_{—} | 25.4 | 1.8 | 14 |
| | (Asymmetric) | S267A | V366L_S267A_{—} | | | |
| | | | H268D | | | |

¹Fold change in affinity over wild-type
²Selectivity is defined as IIb-Fold/IIaR-Fold

### Example 4: Deconvolution of Top Combinations—Lead Variants Generation 1 (LVG1)

Additional variants based on the three top combination variants identified in Example 3 (v19544, v19585 and v19540; see Table 3.2) were developed. These variants were designed to:
a) evaluate the contribution of each mutation in the combinations (deconvolution),
b) evaluate variations in the amino acid substitution at some of the mutated positions,
c) combine strategies (e.g. IgG4 and asymmetric),
d) add other mutations identified as increasing selectivity in Example 1,
e) replace Template 1 (loop replacement) with other loop replacements identified in Example 2, and/or
f) remove potential deamidation sites.

Variants were constructed in a one-armed antibody scaffold (Scaffold 2) and tested for FcγR binding by SPR as described in the General Methods (Protocol 2). The results are shown in Tables 4.1-4.4.

Variants v19544, v19585 and v19540 were also constructed in a full-size antibody format (Scaffold 3) and tested for FcγR binding by SPR as described in the General Methods (Protocol 2). The results are shown in Table 4.5.

TABLE 4.1A
Deconvolution of v19544¹

| | Chain A | Chain B | IIb | IIaR | IIb- | IIaR- | IIb/IIaR | |
|---|---|---|---|---|---|---|---|---|
| Variant # | Mutations | Mutations | KD/M | KD/M | fold² | fold² | Selectivity³ | Comments |
| 19544 | L234D_{—} | Template1_G236D_{—} | 2.80E−08 | 1.00E−07 | 54.9 | 3.7 | 14.8 | Starting |
| | G236N | S239D_S267I_{—} | | | | | | variant |
| | | H268D | | | | | | |
| 22071 | L234D_{—} | Template1_G236D_{—} | 6.3E−08 | 2.70E−07 | 24.4 | 1.4 | 17.1 | Lacking |
| | G236N | S239D_H268D | | | | | | B_S267I. |
| | | | | | | | | Decrease in |
| | | | | | | | | affinity. |
| | | | | | | | | Increase in |
| | | | | | | | | selectivity. |
| 22072 | L234D_{—} | Template1_G236D_{—} | 3.70E−08 | 1.40E−07 | 41.2 | 2.7 | 15.2 | Lacking |
| | G236N | S239D_S267I | | | | | | B_H268D. |
| | | | | | | | | Small |
| | | | | | | | | decrease in |
| | | | | | | | | affinity. |
| | | | | | | | | Selectivity |
| | | | | | | | | unchanged. |
| 19544 | L234D_{—} | Template1_G236D_{—} | 3.20E−08 | 9.70E−08 | 54.9 | 3.7 | 14.8 | Starting |
| | G236N | S239D_S267I_{—} | | | | | | variant |
| | | H268D | | | | | | |
| 22070 | L234D_{—} | Template1_S239D_{—} | 2.0E−08 | 3.80E−08 | 82.8 | 12.5 | 6.6 | Lacking |
| | G236N | S2671_H268D | | | | | | B_G236D. |
| | | | | | | | | Decrease in |
| | | | | | | | | selectivity. |
| 22068 | G236N | Template1_G236D_{—} | 4.4E−09 | 1.20E−08 | 370.6 | 39 | 9.5 | Lacking |
| | | S239D_S267I_{—} | | | | | | A_L234D. |
| | | H268D | | | | | | Small |
| | | | | | | | | decrease in |
| | | | | | | | | selectivity. |
| | | | | | | | | Large |
| | | | | | | | | increase in |
| | | | | | | | | affinity. |
| 22069 | L234D | Template1_G236D_{—} | 5.00E−08 | 1.00E−07 | 32.6 | 4.7 | 7 | Lacking |
| | | S239D_S267I_{—} | | | | | | A_G236N. |
| | | H268D | | | | | | Decrease in |
| | | | | | | | | selectivity. |

¹Data for entries 1-4 were generated in different experiments to data for remaining entries
²Fold change in affinity over wild-type
³Selectivity is defined as IIb-Fold/IIaR-Fold

TABLE 4.1B
Deconvolution of v19544¹

| | Chain A | Chain B | IIb- | IIaR- | IIb/IIaR | |
|---|---|---|---|---|---|---|
| Variant # | Mutations | Mutations | Fold² | Fold² | Selectivity³ | Comments |
| 16463 | Wild-type | Wild-type | 1 | 1 | 1 | |
| | control OAA | control OAA | | | | |
| 19438 | Template 1_WT | | 20.4 | 13.4 | 1.5 | Small increase |
| | | | | | | in selectivity. |
| | | | | | | Increase in |
| | | | | | | affinity. |
| 18370 | S239D | | 4.39 | 3.8 | 1.2 | |
| 19403 | Template 1 + | | 77 | 43.4 | 1.8 | Mutations are |
| | S239D | | | | | additive |

¹Data were generated in a different experiment to data for Tables 4.1A and 4.1C
^{2, 3}See footnotes to Table 4.1A

TABLE 4.1C
Deconvolution of v19544¹

| | Chain A | Chain B | IIb- | IIaR- | IIb/IIaR | |
|---|---|---|---|---|---|---|
| Variant # | Mutations | Mutations | Fold² | Fold² | Selectivity³ | Comments |
| 19535 | L234D_G236N | G236D_S239D_{—} | 2.8 | 0.3 | 8.7 | |
| | | S267I_H268D_{—} | | | | |
| 19438 | | Template 1 | 20.4 | 13.4 | 1.5 | |
| 19544 | L234D_G236N | G236D_S239D_{—} | 54.9 | 3.7 | 14.8 | Mutations are |
| | | S267I_H268D + | | | | additive⁴ |
| | | Template 1 | | | | |

¹Data were generated in a different experiment to data for Tables 4.1A and 4.1B
^{2,3}See footnotes to Table 4.1A
⁴Expected values if effects were additive are: IIb Binding = 57.1; IIaR Binding = 4.0; IIb/IIaR Selectivity = 14.2

TABLE 4.2
Deconvolution of v19540

| | Chain A | Chain B | IIb | IIaR- | IIb/IIaR | |
|---|---|---|---|---|---|---|
| Variant # | Mutations | Mutations | Fold¹ | Fold¹ | Selectivity² | Comments |
| 19540 | L234D_G236N_{—} | G236D_S239D_{—} | 25.4 | 1.8 | 14 | Starting variant |
| | S267A | V266L_S267A_{—} | | | | |
| | | H268D | | | | |
| 22073 | G236N_S267A | G236D_S239D_{—} | 134.8 | 17.3 | 7.8 | Lacking A_L234D. |
| | | V266L_S267A_{—} | | | | Decrease in both |
| | | H268D | | | | affinity and |
| | | | | | | selectivity. |
| 22074 | L234D_S267A | G236D_S239D_{—} | 24.7 | 3.8 | 6.4 | Lacking A_G236N. |
| | | V266L_S267A_{—} | | | | Decrease in |
| | | H268D | | | | selectivity. |
| 22075 | L234D_G236N | G236D_S239D_{—} | 41.1 | 2.7 | 15.1 | Lacking A_S267A. |
| | | V266L_S267A_{—} | | | | Increase in affinity. |
| | | H268D | | | | Selectivity |
| | | | | | | unchanged. |
| 22076 | L234D_G236N_{—} | S239D_V266L_{—} | 21.6 | 6.1 | 3.5 | Lacking B_G236D. |
| | S267A | S267A_H268D | | | | Significant decrease |
| | | | | | | in selectivity. |
| 22077 | L234D_G236N_{—} | G236D_S239D_{—} | 13.4 | 1.5 | 8.9 | Lacking B_V266L. |
| | S267A | S267A_H268D | | | | Decrease in both |
| | | | | | | selectivity and |
| | | | | | | affinity. |
| 22078 | L234D_G236N_{—} | G236D_S239D_{—} | 13.7 | 1.3 | 10.3 | Lacking B_S267A. |
| | S267A | V266L_H268D | | | | Decrease in both |
| | | | | | | selectivity and |
| | | | | | | affinity. |
| 22079 | L234D_G236N_{—} | G236D_S239D_{—} | 12.4 | 1.4 | 9 | Lacking B_H268D. |
| | S267A | V266L_S267A | | | | Decrease in both |
| | | | | | | selectivity and |
| | | | | | | affinity. |

¹Fold change in affinity over wild-type
²Selectivity is defined as IIb-Fold/IIaR-Fold

TABLE 4.3
Deconvolution of v19585¹

| | Chain A | Chain B | IIb- | IIaR- | IIb/IIaR | |
|---|---|---|---|---|---|---|
| Variant # | Mutations | Mutations | Fold² | Fold² | Selectivity³ | Comments |
| 19585 | L234F_G236N_{—} | L234F_G236D_{—} | 49.5 | 4.1 | 12.1 | Starting variant |
| | H268Q_K274Q_{—} | S239D_V266L_{—} | | | | |
| | A327G_A330K_{—} | S267A_H268D_{—} | | | | |
| | P331S | K274Q_A327G_{—} | | | | |
| | | A330S_P331S | | | | |
| 22084 | L234F_G236N_{—} | L234F_G236D_{—} | 34.2 | 2.9 | 11.9 | Lacking A_A330K. |
| | H268Q_K274Q_{—} | S239D_V266L_{—} | | | | Decrease in affinity. |
| | A327G_P331S | S267A_H268D_{—} | | | | Selectivity |
| | | K274Q_A327G_{—} | | | | unchanged. |
| | | A330S_P331S | | | | |
| 22094 | L234F_G236N_{—} | L234F_G236D_{—} | 41.7 | 3.3 | 12.7 | Lacking B_P331S. |
| | H268Q_K274Q_{—} | S239D_V266L_{—} | | | | No significant |
| | A327G_A330K_{—} | S267A_H268D_{—} | | | | effect. |
| | P331S | K274Q_A327G_{—} | | | | |
| | | A330S | | | | |
| 19585 | L234F_G236N_{—} | L234F_G236D_{—} | 44.6 | 5.4 | 8.3 | Starting variant |
| | H268Q_K274Q_{—} | S239D_V266L_{—} | | | | |
| | A327G_A330K_{—} | S267A_H268D_{—} | | | | |
| | P331S | K274Q_A327G_{—} | | | | |
| | | A330S_P331S | | | | |
| 22080 | G236N_H268Q_{—} | L234F_G236D_{—} | 100.5 | 15.5 | 6.5 | Lacking A_L234F. |
| | K274Q_A327G_{—} | S239D_V266L_{—} | | | | Increase in affinity. |
| | A330K_P331S | S267A_H268D_{—} | | | | Decrease in |
| | | K274Q_A327G_{—} | | | | selectivity. |
| | | A330S_P331S | | | | |
| 22081 | L234F_H268Q_{—} | L234F_G236D_{—} | 123.3 | 14.3 | 8.6 | Lacking A_G236N. |
| | K274Q_A327G_{—} | S239D_V266L_{—} | | | | Increase in affinity. |
| | A330K_P331S | S267A_H268D_{—} | | | | Selectivity |
| | | K274Q_A327G_{—} | | | | unchanged. |
| | | A330S_P331S | | | | |
| 22082 | L234F_G236N_{—} | L234F_G236D_{—} | 46.6 | 5.5 | 8.5 | Lacking A_K274Q. |
| | H268Q_A327G_{—} | S239D_V266L_{—} | | | | No significant |
| | A330K_P331S | S267A_H268D_{—} | | | | effect. |
| | | K274Q_A327G_{—} | | | | |
| | | A330S_P331S | | | | |
| 22083 | L234F_G236N_{—} | L234F_G236D_{—} | 39.5 | 4 | 10 | Lacking A_A327G. |
| | H268Q_K274Q_{—} | S239D_V266L_{—} | | | | Slight increase in |
| | A330K_P331S | S267A_H268D_{—} | | | | selectivity. |
| | | K274Q_A327G_{—} | | | | |
| | | A330S_P331S | | | | |
| 22085 | L234F_G236N_{—} | L234F_G236D_{—} | 79.2 | 9.4 | 8.4 | Lacking A_P331S. |
| | H268Q_K274Q_{—} | S239D_V266L_{—} | | | | Increase in affinity. |
| | A327G_A330K | S267A_H268D_{—} | | | | Selectivity |
| | | K274Q_A327G_{—} | | | | unchanged. |
| | | A330S_P331S | | | | |
| 22086 | L234F_G236N_{—} | G236D_S239D_{—} | 55.1 | 5.6 | 9.7 | Lacking B_L234F. |
| | H268Q_K274Q_{—} | V266L_S267A_{—} | | | | Increase in both |
| | A327G_A330K_{—} | H268D_K274Q_{—} | | | | affinity and |
| | P331S | A327G_A330S_{—} | | | | selectivity. |
| | | P331S | | | | |
| 22130 | L234F_G236N_{—} | L234F_S239D_{—} | 52.6 | 16.8 | 3.1 | Lacking B_G236D. |
| | H268Q_K274Q_{—} | V266L_S267A_{—} | | | | Decrease in |
| | A327G_A330K_{—} | H268D_K274Q_{—} | | | | selectivity. |
| | P331S | A327G_A330S_{—} | | | | |
| | | P331S | | | | |
| 22088 | L234F_G236N_{—} | L234F_G236D_{—} | 44.7 | 6.5 | 6.9 | Lacking B_V266L. |
| | H268Q_K274Q_{—} | S239D_S267A_{—} | | | | Decrease in |
| | A327G_A330K_{—} | H268D_K274Q_{—} | | | | selectivity. |
| | P331S | A327G_A330S_{—} | | | | |
| | | P331S | | | | |
| 22089 | L234F_G236N_{—} | L234F_G236D_{—} | 27.8 | 3 | 9.2 | Lacking B_S267A. |
| | H268Q_K274Q_{—} | S239D_V266L_{—} | | | | Decrease in affinity. |
| | A327G_A330K_{—} | H268D_K274Q_{—} | | | | Selectivity |
| | P331S | A327G_A330S_{—} | | | | unchanged. |
| | | P331S | | | | |
| 22090 | L234F_G236N_{—} | L234F_G236D_{—} | 27.7 | 3.2 | 8.8 | Lacking B_H268D. |
| | H268Q_K274Q_{—} | S239D_V266L_{—} | | | | Decrease in affinity. |
| | A327G_A330K_{—} | S267A_K274Q_{—} | | | | Selectivity |
| | P331S | A327G_A330S_{—} | | | | unchanged. |
| | | P331S | | | | |
| 22091 | L234F_G236N_{—} | L234F_G236D_{—} | 41.4 | 4.6 | 9 | Lacking B_K274Q. |
| | H268Q_K274Q_{—} | S239D_V266L_{—} | | | | No significant |
| | A327G_A330K_{—} | S267A_H268D_{—} | | | | effect. |
| | P331S | A327G_A330S_{—} | | | | |
| | | P331S | | | | |
| 22092 | L234F_G236N_{—} | L234F_G236D_{—} | 77.1 | 8 | 9.7 | Lacking B_A327G. |
| | H268Q_K274Q_{—} | S239D_V266L_{—} | | | | Increase in affinity. |
| | A327G_A330K_{—} | S267A_H268D_{—} | | | | Selectivity |
| | P331S | K274Q_A330S_{—} | | | | unchanged. |
| | | P331S | | | | |
| 22093 | L234F_G236N_{—} | L234F_G236D_{—} | 43.6 | 5.2 | 8.5 | Lacking B_A330S. |
| | H268Q_K274Q_{—} | S239D_V266L_{—} | | | | No significant |
| | A327G_A330K_{—} | S267A_H268D_{—} | | | | effect. |
| | P331S | K274Q_A327G_{—} | | | | |
| | | P331S | | | | |

¹Data for entries 1-3 were generated in a different experiment to data for remaining entries
²Fold change in affinity over wild-type
³Selectivity is defined as IIb-Fold/IIaR-Fold

TABLE 4.4
Combinations, Variations and Other Loop Templates¹

| Variant | Chain A | Chain B | IIb- | IIaR- | IIb/IIaR | |
|---|---|---|---|---|---|---|
| # | Mutations | Mutations | Fold² | Fold² | Selectivity³ | Comments |
| 19544 | L234D_G236N | Template1_G236D_{—} | 54.9 | 3.7 | 14.8 | Starting variant |
| | | S239D_S267I_{—} | | | | |
| | | H268D | | | | |
| 19585 | L234F_G236N_{—} | L234F_G236D_{—} | 49.5 | 4.1 | 12.1 | Starting variant |
| | H268Q_K274Q_{—} | S239D_V266L_{—} | | | | |
| | A327G_A330K_{—} | S267A_H268D_{—} | | | | |
| | P331S | K274Q_A327G_{—} | | | | |
| | | A330S_P331S | | | | |
| 19540 | L234D_G236N_{—} | G236D_S239D_{—} | 25.4 | 1.8 | 14 | Starting variant |
| | S267A | V266L_S267A_{—} | | | | |
| | | H268D | | | | |
| 22098 | L234D_G236N | Template1_G236D_{—} | 47.4 | 3 | 15.7 | v19544/B_S267I → A. |
| | | S239D_S267A_{—} | | | | Minor change in |
| | | H268D | | | | selectivity. Use of |
| | | | | | | ALA should reduce |
| | | | | | | hydrophobicity. |
| 22101 | L234D_G236N | Template1_G236D_{—} | 30.3 | 1.8 | 17 | v19544/+B_V266L, |
| | | S239D_V266L_{—} | | | | B_S267I → A. |
| | | S267A_H268D | | | | Small synergistic |
| | | | | | | effect on selectivity. |
| 22103 | L234F_G236N_{—} | L234F_V266L_{—} | 40.6 | 2.8 | 14.6 | v19544 + v19585/ |
| | H268Q_K274Q_{—} | H268D_K274Q_{—} | | | | A_L234D → F. |
| | A327G_A330K_{—} | Template1_G236D_{—} | | | | No significant |
| | P331S | S239D_S267I_{—} | | | | effect. |
| | | H268D | | | | |
| 22107 | L234D_G236N | Template1_G236N_{—} | 45 | 2.9 | 15.7 | v19544/B_G236D → N. |
| | | S239D_S267I_{—} | | | | Slight decrease in |
| | | H268D | | | | affinity. Selectivity |
| | | | | | | unchanged. |
| 22118 | L234F_G236N_{—} | L234F_G236D_{—} | 46.6 | 2.5 | 18.9 | v19585/−B_A327G_A330S_{—} |
| | H268Q_K274Q_{—} | S239D_V266L_{—} | | | | P331S; +B_Template 1. |
| | A327G_A330K_{—} | S267A_H268D_{—} | | | | Slight increase in |
| | P331S | K274Q_Template 1 | | | | selectivity. |
| 19544 | L234D_G236N | Template1_G236D_{—} | 54.9 | 3.7 | 14.8 | Starting variant |
| | | S239D_S267I_{—} | | | | |
| | | H268D | | | | |
| 19585 | L234F_G236N_{—} | L234F_G236D_{—} | 49.5 | 4.1 | 12 | Starting variant |
| | H268Q_K274Q_{—} | S239D_V266L_{—} | | | | |
| | A327G_A330K_{—} | S267A_H268D_{—} | | | | |
| | P331S | K274Q_A327G_{—} | | | | |
| | | A330S_P331S | | | | |
| 19540 | L234D_G236N_{—} | G236D_S239D_{—} | 25.4 | 1.8 | 14 | Starting variant |
| | S267A | V266L_S267A_{—} | | | | |
| | | H268D | | | | |
| 22095 | L234D_G236N | Template7_G236D_{—} | 7.5 | 0.8 | 9.4 | v19544/Template |
| | | S239D_S267I_{—} | | | | 1 → 7. |
| | | H268D | | | | Decrease in affinity |
| | | | | | | and selectivity. |
| 22096 | L234D_G236N | Template66_{—} | 3.5 | 0.3 | 11 | v19544/Template |
| | | G236D_S239D_{—} | | | | 1 → 66. |
| | | S267I_H268D | | | | Decrease in affinity |
| | | | | | | and selectivity. |
| 22097 | L234D_G236N | Template151_{—} | 21.5 | 2.3 | 9.5 | v19544/Template |
| | | G236D_S239D_{—} | | | | 1 → 151. |
| | | S267I_H268D | | | | Decrease in affinity |
| | | | | | | and selectivity. |
| 22099 | L234D_G236N_{—} | Template1_G236D_{—} | 31.5 | 2.4 | 13.3 | v19544/+A_S267A. |
| | S267A | S239D_S267I_{—} | | | | Decrease in affinity. |
| | | H268D | | | | No significant |
| | | | | | | change in |
| | | | | | | selectivity. |
| 22100 | L234D_G236N | Template1_G236D_{—} | 17.5 | 1.3 | 13.9 | v19544/+B_V266L. |
| | | S239D_V266L_{—} | | | | No significant |
| | | S267I_H268D | | | | change in |
| | | | | | | selectivity. |
| 22102 | L234D_G236N_{—} | L234F_V266L_{—} | 20.9 | 1.7 | 12.5 | v19544 + v19585. |
| | H268Q_K274Q_{—} | H268D_K274Q_{—} | | | | No significant |
| | A327G_A330K_{—} | Template1_G236D_{—} | | | | change in |
| | P331S | S239D_S267I_{—} | | | | selectivity. |
| | | H268D | | | | |
| 22104 | L234D_G236N_{—} | Template1_G236D_{—} | 75 | 6.3 | 11.9 | v19544/+A_A330K. |
| | A330K | S239D_S267I_{—} | | | | Increase in affinity. |
| | | H268D | | | | No significant |
| | | | | | | change in |
| | | | | | | selectivity. |
| 22105 | L234D_G236N_{—} | Template1_G236D_{—} | 40.4 | 3.9 | 10.3 | v19544/+A_K326D. |
| | K326D | S239D_S267I_{—} | | | | No significant |
| | | H268D | | | | effect. |
| 22106 | L234D_G236N_{—} | Template1_G236D_{—} | 66.8 | 5.7 | 11.6 | v19544/+A_K326D; +A_A330K. |
| | K326D_A330K | S239D_S267I_{—} | | | | Same effect |
| | | H268D | | | | as +A_A330K |
| | | | | | | alone. |
| 22108 | L234D_G236D | Template1_G236D_{—} | 21.7 | 4.1 | 5.3 | v19544/A_G236N → D. |
| | | S239D_S267I_{—} | | | | Decrease in |
| | | H268D | | | | selectivity |
| 22109 | L234D_G236N | Template1_G236D_{—} | 123.6 | 10.4 | 11.9 | v19544/B_S267I → V. |
| | | S239D_S267V_{—} | | | | Increase in |
| | | H268D | | | | affinity. |
| 22110 | L234D_G236N | Template1_G236D_{—} | 162.2 | 10.2 | 15.9 | v19544/+B_G237F. |
| | | G237F_S239D_{—} | | | | Removes |
| | | S267I_H268D | | | | deamidation site on |
| | | | | | | chain B. Increase in |
| | | | | | | affinity. |
| 22121 | L234D_G236N_{—} | Template1_G236D_{—} | 64.3 | 6.4 | 10.1 | v19544/+A_G237A. |
| | G237A | S239D_S267I_{—} | | | | Removes |
| | | H268D | | | | deamidation site on |
| | | | | | | chain A. Increase in |
| | | | | | | affinity. Decrease in |
| | | | | | | selectivity. |
| 22122 | L234D_G236N_{—} | Template1_G236D_{—} | 158.5 | 12 | 13.2 | v19544/+A_G237A; +B_G237F. |
| | G237A | G237F_S239D_{—} | | | | Removes |
| | | S267I_H268D | | | | deamidation sites on |
| | | | | | | chain A and chain |
| | | | | | | B. Increase in |
| | | | | | | affinity. |
| 22112 | L234D_G236D_{—} | Template1_G236D_{—} | 207.1 | 41.6 | 5 | v19544/A_G236N → |
| | G237F | G237F_S239D_{—} | | | | D; +A_G237F; +B_G237F. |
| | | S267I_H268D | | | | Increase in affinity. |
| | | | | | | Decrease in |
| | | | | | | selectivity. |
| 22113 | L234D_G236D_{—} | Template1_G236D_{—} | 105.2 | 36.4 | 2.9 | v19544/A_G236N → |
| | G237W | G237W_S239D_{—} | | | | D; +A_G237W; +B_G237W. |
| | | S267I_H268D | | | | Increase in affinity. |
| | | | | | | Decrease in |
| | | | | | | selectivity. |
| 22131 | L234F_G236N_{—} | G236D_S239D_{—} | 45.1 | 4.3 | 10.4 | v19540/A_L234D → F. |
| | S267A | V266L_S267A_{—} | | | | Increase in affinity. |
| | | H268D | | | | Decrease in |
| | | | | | | selectivity. |
| 22115 | L234D_G236N_{—} | G236D_S239D_{—} | 2.6 | 0.4 | 5.9 | v19540 + v19585. |
| | S267A_H268Q_{—} | V266L_S267I_{—} | | | | Decrease in affinity |
| | K274Q_A327G_{—} | H268D_L234F_{—} | | | | and selectivity. |
| | A330K_P331S | K274Q_A327G_{—} | | | | |
| | | A330K_P331S | | | | |
| 22116 | L234D_G236N_{—} | G236D_S239D_{—} | 31.9 | 2.6 | 12.1 | v19540/+A_K326D; +B_K326D. |
| | S267A_K326D | V266L_S267A_{—} | | | | Increase in |
| | | H268D_K326D | | | | affinity. |
| 22117 | L234D_G236N_{—} | L234F_G236D_{—} | 28.7 | 2.3 | 12.7 | v19585/A_L234F → D. |
| | H268Q_K274Q_{—} | S239D_V266L_{—} | | | | Decrease in |
| | A327G_A330K_{—} | S267A_H268D_{—} | | | | affinity. |
| | P331S | K274Q_A327G_{—} | | | | |
| | | A330S_P331S | | | | |
| 22119 | L234F_G236N_{—} | L234F_G236D_{—} | 6.2 | 0.8 | 7.9 | v19585/B_S267A → I. |
| | H268Q_K274Q_{—} | S239D_V266L_{—} | | | | Decrease in both |
| | A327G_A330K_{—} | S267I_H268D_{—} | | | | affinity and |
| | P331S | K274Q_A327G_{—} | | | | selectivity. |
| | | A330S_P331S | | | | |
| 22120 | L234F_G236N_{—} | L234F_G236D_{—} | 85.8 | 9.7 | 8.8 | v19585/+A_K326D; +B_K326D. |
| | H268Q_K274Q_{—} | S239D_V266L_{—} | | | | Increase in |
| | A327G_A330K_{—} | S267A_H268D_{—} | | | | affinity. |
| | P331S_K326D | K274Q_A327G_{—} | | | | |
| | | A330S_P331S_{—} | | | | |
| | | K326D | | | | |

¹Data for entries 1-8 were generated in a different experiment to data for remaining entries
²Fold change in affinity over wild-type
³Selectivity is defined as IIb-Fold/IIaR-Fold

TABLE 4.5
Full-size Antibody (FSA) Formats

| Variant | Chain A | Chain B | IIb- | IIaR- | IIb/IIaR | |
|---|---|---|---|---|---|---|
| # | Mutations | Mutations | Fold¹ | Fold¹ | Selectivity² | Comments |
| 19544 | L234D_G236N | Template1_G236D_{—} | 54.9 | 3.7 | 15 | Starting variant |
| | | S239D_S267I_{—} | | | | |
| | | H268D | | | | |
| 22126 | L234D_G236N | Template1_G236D_{—} | 21.1 | 1.9 | 11 | FSA format of |
| | | S239D_S267I_{—} | | | | v19544. Decrease in |
| | | H268D | | | | affinity and |
| | | | | | | selectivity. |
| 19540 | L234D_G236N_{—} | G236D_S239D_{—} | 25.4 | 1.8 | 14 | Starting variant |
| | S267A | V266L_S267A_{—} | | | | |
| | | H268D | | | | |
| 22127 | L234D_G236N_{—} | G236D_S239D_{—} | 23.9 | 1.6 | 15.2 | FSA format of |
| | S267A | V266L_S267A_{—} | | | | v19540. No |
| | | H268D | | | | significant change in |
| | | | | | | affinity or |
| | | | | | | selectivity. |
| 19585 | L234F_G236N_{—} | L234F_G236D_{—} | 49.5 | 4.1 | 12 | Starting variant |
| | H268Q_K274Q_{—} | S239D_V266L_{—} | | | | |
| | A327G_A330K_{—} | S267A_H268D_{—} | | | | |
| | P331S | K274Q_A327G_{—} | | | | |
| | | A330S_P331S | | | | |
| 22128 | L234F_G236N_{—} | L234F_G236D_{—} | 37.4 | 3.3 | 11.3 | FSA format of |
| | H268Q_K274Q_{—} | S239D_V266L_{—} | | | | v19585. No |
| | A327G_A330K_{—} | S267A_H268D_{—} | | | | significant change in |
| | P331S | K274Q_A327G_{—} | | | | affinity or |
| | | A330S_P331S | | | | selectivity. |

¹Fold change in affinity over wild-type
²Selectivity is defined as IIb-Fold/IIaR-Fold

### CONCLUSIONS

The Tables above show the mutations made to the initial variants (v19544, v19540 and v19585) had differing effects on the FcγRIIb affinity and/or selectivity of the variant as outlined in more detail below. All tested variants, however, still retained a FcγRIIb selectivity that was higher than wild-type.

### Variant 19544

The effect of Template 1 (loop replacement) on FcγRIIb selectivity is additive
Chain B_G236D is important for FcγRIIb selectivity
The effect of Chain B_S239D on FcγRIIb selectivity is generally additive
Chain B_S267I added affinity for FcγRIIb, but appeared to decrease selectivity
Chain B_H268D enhances binding to FcγRIIb (binding enhancer)

### Variant 19540

Removal of Chain A_L234D decreases FcγRIIb selectivity, but increases FcγRIIb affinity
Removal of Chain A_G236N or Chain B_G236D decreases FcγRIIb selectivity and, to a lesser extent, FcγRIIb affinity
The effect of Chain B_S239D on FcγRIIb selectivity is generally additive
Removal of Chain B_V266L, Chain B_S267A or Chain B_H268D decreases both FcγRIIb selectivity and affinity

### Variant 19585

Favourable or mute mutations are: Chain B_L234F, Chain B_K274Q, Chain B_A330S, Chain B_A330S and Chain B_P331S. One or more of these mutations could likely be omitted.
Important mutations are: Chain A_L234F, Chain A_G236N, Chain A_K274Q, Chain A_A327G, Chain A_A330K, Chain A_P331S, Chain B_G236D, Chain B_V266L, Chain B_S267A and Chain B_H268D.

### Example 5: Stability Mutations

Six individual mutations (A287F, M428F, T250V, L309Q, L242C_I336C and V308I) that improved thermostability of the Fc were identified in a trastuzumab homodimer background. These individual mutations were ported into two different heterodimeric trastuzumab FcγRIIb selective variants (v27293 and v27294—see Table 5.1) to assess their compatibility with CH2 mutations that improve FcγRIIb selectivity. Both v27293 and v27294 were in one-armed antibody format (Scaffold 2).

Additionally, six combinations of two or three stability-enhancing mutations (A287F/M428F, A287F/T250V, M428F/T250V, A287F/M428F/T250V, T250V/L309Q and L242C_I336C/V308I) were tested to assess if increased stabilization could be obtained by additive or synergistic effects.

Twenty-four variants were constructed as described in the General Methods, each including stability- and selectivity-enhancing mutations as shown in Tables 5.1 and 5.2. Variants were assessed for expression, aggregation, thermal stability and binding affinity for FcγRIIb, FcγRIIa and FcγRI as described in the General Methods.

Certain variants were excluded from further characterization based on analytical SEC profiles. The area under the curve of the chromatogram was integrated for all signal present and converted to a percentage of each species present in the variant sample. The percentage of high molecular weight (HMW) species observed in the analytical SEC profiles indicates the abundance of full-size antibody formed for each variant using a single DNA ratio for expression. Variants with less than 20% HMW species upon expression at a single DNA ratio were considered successful. Only 3 variants had more than 20% HMNW species (see Table 5.2) and were not included in further characterization. Low molecular weight (LMVW) species indicates the presence of mis-paired Fc homodimer, which doesn't interfere with determination of the Tm, or with the binding affinity for any of the FcγRs.

TABLE 5.1
Parental Variants Used to Assess Stability-Enhancing Mutations

| Parental | |
|---|---|
| Variant | CH2 Mutations |
| v27293 | Chain A: |
| | G236N_G237A |
| | Chain B: |
| | G236D_G237F_S239D_S267V_H268D_Template 1 |
| v27294 | Chain A: |
| | L234F_G236N_H268Q_A327G_A330K_P331S |
| | Chain B: |
| | G236D_S239D_V266L_S267A_H268D |

TABLE 5.2
Effect of Stability-Enhancing Mutations on Aggregation and Tm

| | | | | Retention | Tₘ | | |
|---|---|---|---|---|---|---|---|
| | % | % | % | Time | CH₂ | | Theoretical |
| Variant Description | HMW¹ | Heterodimer¹ | LMW¹ | (min)¹ | (° C.)² | ΔTₘ³ | ΔTₘ⁴ |
| v27293_Parental | 3.1 | 88.1 | 8.8 | 7.92 | 59.0 | 0.0 | |
| v27293_A287F | 2.6 | 91.7 | 5.7 | 7.96 | 62.5 | 3.5 | |
| v27293_M428F | 2.8 | 90.1 | 7.1 | 7.95 | 61.0 | 2.0 | |
| v27293_T250V | 2.9 | 90.3 | 6.8 | 7.91 | 64.5 | 5.5 | |
| v27293_L309Q | 3.5 | 89.7 | 6.8 | 7.96 | 61.0 | 2.0 | |
| v27293_{—} | >20 | | | | N/A⁵ | N/A | N/A |
| L242C_I336C | | | | | | | |
| v27293_V308I | 2.8 | 93.8 | 3.4 | 7.96 | 59.5 | 0.5 | |
| v27293_{—} | 1.7 | 82.0 | 16.3 | 7.96 | 65.5 | 6.5 | 5.5 |
| A287F/M428F | | | | | | | |
| v27293_{—} | 1.8 | 86.3 | 11.9 | 7.96 | 68.0 | 9.0 | 9.0 |
| A287F/T250V | | | | | | | |
| v27293_{—} | 3.6 | 88.0 | 8.5 | 7.96 | 67.5 | 8.5 | 7.5 |
| M428F/T250V | | | | | | | |
| v27293_{—} | >20 | | | | N/A | N/A | N/A |
| A287F/M428F/T250V | | | | | | | |
| v27293_{—} | 2.9 | 93.1 | 4.1 | 7.94 | 68.0 | 9.0 | 7.5 |
| T250V/L309Q | | | | | | | |
| v27293_{—} | 19.6 | 75.0 | 5.4 | 7.94 | 62.0 | 3.0 | 0.5 |
| L242C_I336C/V308I | | | | | | | |
| v27294_Parental | 3.4 | 94.6 | 1.9 | 7.94 | 62.0 | 0.0 | |
| v27294_A287F | 3.0 | 94.6 | 2.4 | 7.98 | 66.0 | 4.0 | |
| v27294_M428F | 4.3 | 87.4 | 8.4 | 7.92 | 63.0 | 1.0 | |
| v27294_T250V | 3.0 | 93.2 | 3.8 | 7.93 | 67.5 | 5.5 | |
| v27294_L309Q | 4.8 | 87.2 | 8.0 | 7.95 | 64.5 | 2.5 | |
| v27294_{—} | 1.6 | 88.6 | 9.9 | 7.98 | 62.0 | 0.0 | |
| L242C_I336C | | | | | | | |
| v27294_V308I | 4.5 | 89.6 | 5.9 | 7.98 | 63.0 | 1.0 | |
| v27294_{—} | 2.5 | 88.3 | 9.2 | 7.97 | 69.0 | 7.0 | 5.0 |
| A287F/M428F | | | | | | | |
| v27294_{—} | 2.7 | 89.2 | 8.1 | 7.97 | 71.5 | 9.5 | 9.5 |
| A287F/T250V | | | | | | | |
| v27294_{—} | 9.6 | 75.9 | 14.5 | 7.98 | 60.0 | −2.0 | 6.5 |
| M428F/T250V | | | | | | | |
| v27294_{—} | >20 | | | | N/A | N/A | N/A |
| A287F/M428F/T250V | | | | | | | |
| v27294_{—} | 4.6 | 89.5 | 5.9 | 7.96 | 70.5 | 8.5 | 8.0 |
| T250V/L309Q | | | | | | | |
| v27294_{—} | 2.0 | 86.1 | 11.9 | 8 | 62.5 | 0.5 | 1.0 |
| L242C_I336C/V308I | | | | | | | |

¹% HMW, % heterodimer, % LMW and retention time of monomer all pertain to the profile observed by analytical SEC for each variant and indicates their relative abundance. % HMW corresponds to mis-paired full-size antibody, % heterodimer corresponds to heterodimer one-armed antibody and % LMW corresponds to mis-paired homodimeric Fc
²The transition observed for the CH₂ domain by DSF is reported
³ΔTₘ indicates the difference between the Tₘ mutated − Tₘ parental (v27923 or v27924)
⁴Theoretical ΔTₘ implies an additive stabilization effect based on the point mutation in the respective parental variant
⁵N/A indicates data was not collected due to low purity of the samples

TABLE 5.3
Effect of Stability-Enhancing Mutations on FcγRIIb Selectivity

| | K_{D} | K_{D} | K_{D} | K_{D} |
|---|---|---|---|---|
| | FcγRIIb | FcγRIIaR | FcγRIIaH | FcγRI |
| Variant Description | (M) | (M) | (M) | (M) |
| v27293_Parental | 3.26E−09 | 1.39E−08 | 2.24E−06 | 2.89E−08 |
| v27293_A287F | 3.66E−09 | 1.53E−08 | 2.13E−06 | 1.67E−08 |
| v27293_M428F | 3.89E−09 | 1.66E−08 | 2.14E−06 | 2.32E−08 |
| v27293_T250V | 3.62E−09 | 1.49E−08 | 2.03E−06 | 1.89E−08 |
| v27293_L309Q | 2.99E−09 | 1.41E−08 | 2.07E−06 | 1.57E−08 |
| v27293_L242C_1336C | N/A¹ | N/A | N/A | N/A |
| v27293_V308I | 5.19E−09 | 2.02E−08 | 2.38E−06 | 1.30E−08 |
| v27293_A287F/M428F | 4.04E−09 | 1.55E−08 | 1.87E−06 | 1.90E−08 |
| v27293_A287F/T250V | 3.26E−09 | 1.37E−08 | 1.88E−06 | 1.68E−08 |
| v27293_M428F/T250V | 4.50E−09 | 1.77E−08 | 1.96E−06 | 1.97E−08 |
| v27293_A287F/M428F/ | N/A | N/A | N/A | N/A |
| T250V | | | | |
| v27293_T250V/L309Q | 4.62E−09 | 1.87E−08 | 2.02E−06 | 2.47E−08 |
| v27293_L242C_1336C/ | 5.24E−09 | 1.90E−08 | 2.16E−06 | 5.34E−09 |
| V308I | | | | |
| v27294_Parental | 1.81E−08 | 6.23E−08 | 6.29E−07 | 6.17E−10 |
| v27294_A287F | 2.33E−08 | 7.72E−08 | 6.01E−07 | 6.21E−10 |
| v27294_M428F | 1.85E−08 | 5.99E−08 | 4.73E−07 | 5.88E−10 |
| v27294_T250V | 1.93E−08 | 6.85E−08 | 5.50E−07 | 5.69E−10 |
| v27294_L309Q | 2.48E−08 | 7.90E−08 | 5.25E−07 | 7.74E−10 |
| v27294_L242C_I336C | 2.30E−08 | 8.28E−08 | 6.13E−07 | 8.18E−10 |
| v27294_V308I | 2.76E−08 | 8.56E−08 | 6.04E−07 | 8.48E−10 |
| v27294_A287F/M428F | 2.60E−08 | 8.20E−08 | 5.60E−07 | 7.21E−10 |
| v27294_A287F/T250V | 2.41E−08 | 7.94E−08 | 5.67E−07 | 6.79E−10 |
| v27294_M428F/T250V | 1.83E−08 | 6.29E−08 | 5.63E−07 | 7.47E−10 |
| v27294_A287F/M428F/ | N/A | N/A | N/A | N/A |
| T250V | | | | |
| v27294_T250V/L309Q | 2.15E−08 | 7.14E−08 | 5.38E−07 | 7.64E−10 |
| v27294_L242C_I336C/ | 1.55E−08 | 6.14E−08 | 5.75E−07 | 5.32E−10 |
| V308I | | | | |

¹N/A indicates data was not collected due to low purity of the samples

Mutations that met the following criteria were considered to be successful stability-enhancing mutations:
a. an increase in Tₘ by DSF>2° C. for a single point mutation and minimally an additive effect when combined
b. retention of wild-type like properties (<2-fold difference compared to parental variant) in terms of FcγRIIb, FcγRIIa and FcγRI binding
c. heterodimer content >75% by analytical SEC.

Successful single mutations for thermostability include: A287F (+3.5-4° C.), T250V (+5.5° C.), L309Q (+2-2.5° C.) and M428F (+1-2° C.).

Stability-enhancing designs with either additive or synergistic contributions include A287F/M428F (+6.5-7° C.), A287F/T250V (+9.0-9.5° C.), M428F/T250V (+8.5° C., −2° C.) and T250V/L309Q (+8.5-9.0° C.). The A287F/M428F and T250V/L309Q combinations yielded an increase in Tₘ slightly higher than additive effect, while A287F/T250V yielded an additive effect.

### Example 6: Optimization of Initial Lead Variants

The following strategies were employed to optimize two of the lead variants, v19544 (Lead 1) and v19585 (Lead 2), identified in Example 4, resulting in the generation of over 1500 variants that were subsequently tested for FcγRIIb selectivity and affinity.
1. Perform a systematic 1× scan of environment residues to optimize Lead 1.
2. Perform a systematic 1× scan of environment residues to optimize Lead 2.
3. Combine hits from loop library with Lead 1.
4. Test longer loop replacements.
5. Combine stability variants with Leads 1 and 2.

Based on the results from Example 4, the following modifications to the lead variants v19544 and v19585 were made and the resulting variants (v27293, v27294 and v27362 as shown in Tables 6.1-6.3) were used as “Launching Modules” for the next round of optimization.

TABLE 6.1
Launching Module 1 for Strategies 1 and 5

| | | | Estimated |
|---|---|---|---|
| Variant | | Chain B | Starting |
| # | Chain A Mutations | Mutations | Selectivity^{§} |
| v19544 | L234D_G236N | G236D_S239D_S267I_H268D_Template 1 | 14 |
| v27293 | G236N_G237A | G236D_G237F_S239D_S267V_H268D_Template 1 | 15-20 |

^{§}IIb Fold over wild-type/IIaR Fold over wild-type

TABLE 6.2
Launching Module 2 for Strategies 2 and 5

| | | | Estimated |
|---|---|---|---|
| Variant | | | Starting |
| # | Chain A Mutations | Chain B Mutations | Selectivity^{§} |
| v19585 | L234F_G236N_H268Q_K274Q_{—} | L234F_G236D_S239D_V266L_{—} | 12 |
| | A327G_A330K_P331S | S267A_H268D_K274Q_A327G_{—} | |
| | | A330S_P331S | |
| v27294 | L234F_G236N_H268Q_A327G_{—} | G236D_S239D_V266L_S267A_{—} | 10-15 |
| | A330K_P331S | H268D | |

^{§}IIb Fold over wild-type/IIaR Fold over wild-type

TABLE 6.3
Launching Module 3 for Strategies 3 and 4

| | | | Estimated |
|---|---|---|---|
| Variant | | | Starting |
| # | Chain A Mutations | Chain B Mutations | Selectivity^{§} |
| v19544 | L234D_G236N | G236D_S239D_S267I_H268D_Template 1 | 14 |
| v27362 | G236N_G237A | G236D_G237F_S239D_S267V_H268D | 15-20 |

^{§}IIb Fold over wild-type/IIaR Fold over wild-type

Variants were constructed in a one-armed antibody scaffold (Scaffold 2) and tested for FcγR binding by SPR as described in the General Methods (Protocol 2). Results are shown in Table 6.4.

TABLE 6.4
FcγR Binding Characteristics of Launching Modules

| | | | Fold Change in | | |
|---|---|---|---|---|---|
| | K_{D}/M | | Affinity over WT | | IIb/IIaR |
| Variant | FcγRIIb | FcγRIIaR | FcγRIIb | FcγRIIaR | Selectivity¹ |
| v16463² | 1.40E−06 | 3.10E−07 | 1 | 1 | 1 |
| v19544 | 3.20E−08 | 8.80E−08 | 45.4 | 3.5 | 13 |
| v27293 | 3.50E−09 | 8.80E−09 | 415.4 | 35.2 | 11.8 |
| v19585 | 4.80E−08 | 1.10E−07 | 30.1 | 2.8 | 10.7 |
| v27294 | 1.60E−08 | 4.40E−08 | 92.9 | 7 | 13.4 |
| v27362 | 1.50E−07 | 2.90E−07 | 9.4 | 1.1 | 8.7 |

¹Selectivity is defined as IIb-Fold/IIaR-Fold
²Wild-type (OAA format)

### Strategy 1

Strategy 1 involved conducting a systematic 1× scan of residues in the environment of the Fc/FcγR interaction to identify those that could potentially further improve the selectivity of the original v19544 design. An in silico 2D-interaction map and structural analysis were employed to identify positions that could influence affinity and/or selectivity of the Fc/FcγR interaction. Mutations compatible with the relevant secondary structure elements were selected for testing. Specifically, residues in loops were mutated to all possible amino acids except cysteine (18 amino acids) and residues in beta sheet positions were mutated with compatible residues (7 amino acids) as shown in Table 6.5. The total number of variants constructed was 471.

TABLE 6.5
Mutations Tested under Strategy 1*

| | | Parent | | No. of |
|---|---|---|---|---|
| | | Amino | Amino Acid | Substi- |
| Chain | Position | Acid | Substitutions | tutions |
| A | 234 | Leu | All except Cys | 18 |
| A | 235 | Leu | All except Cys | 18 |
| A | 236 | Gly | All except Cys | 18 |
| A | 237 | Gly | All except Cys | 18 |
| A | 239 | Ser | All except Cys | 18 |
| B | 234 | Leu | All except Cys | 18 |
| B | 235 | Leu | All except Cys | 18 |
| B | 236 | Gly | All except Cys | 18 |
| B | 237 | Gly | All except Cys | 18 |
| B | 239 | Ser | All except Cys | 18 |
| B | 240 | Val | Ala, Val, Leu, Ile, Met, Phe, Thr | 7 |
| B | 263 | Val | Ala, Val, Leu, Ile, Met, Phe, Thr | 7 |
| B | 264 | Val | Ala, Val, Leu, Ile, Met, Phe, Thr | 7 |
| B | 266 | Val | Ala, Val, Leu, Ile, Met, Phe, Thr | 7 |
| B | 267 | Ser | All except Cys | 18 |
| B | 268 | Asp | All except Cys | 18 |
| B | 269 | Glu | All except Cys | 18 |
| B | 270 | Asp | All except Cys | 18 |
| B | 271 | Pro | All except Cys | 18 |
| B | 273 | Val | Ala, Val, Leu, Ile, Met, Phe, Thr | 7 |
| B | 323 | Val | Ala, Val, Leu, Ile, Met, Phe, Thr | 7 |
| B | 325* | Ser | All except Cys | 18 |
| B | 326* | Thr | All except Cys | 18 |
| B | 327* | Trp | All except Cys | 18 |
| B | 328* | Phe | All except Cys | 18 |
| B | 329* | Asp | All except Cys | 18 |
| B | 330* | Gly | All except Cys | 18 |
| B | 331* | Gly | All except Cys | 18 |
| B | 331*A | Tyr | All except Cys | 18 |
| B | 331*B | Ala | All except Cys | 18 |
| B | 332 | Ile | Ala, Val, Leu, Ile, Met, Phe, Thr | 7 |

*Based on Launching Module 1 (v27293): A: G236N_G237A/B: G236D_G237F_S239D_S267V_H268D_Template 1

Variants were constructed in a one-armed antibody scaffold (Scaffold 2) and tested for FcγR binding by SPR as described in the General Methods (Protocol 2).

The results are summarized in FIGS. 10A & B and described below.

### Affinity—Position 330*

As shown in FIG. 10(A), mutations at position 330* produced the greatest improvement in affinity of FcγRIIb binding. Position 330* is within the loop inserted into variant v19544 and is proximal to position 135 in the receptor (S135 in FcγRIIb and L135 in FcγRIIaR).

Analysis of the different mutations made at position 330* as shown in Table 6.6 reveals the following trends:
i) Hydrophobic mutations tended to increase binding to FcγRIIaR
ii) Exceptions to point i) were G330A/L/I, which each increased binding to both receptors, suggesting a new loop conformation
iii) Hydrophilic mutations tended to increase binding to FcγRIIb.

As described in Example 2, a search of the structures in the PDB to identify residues that could potentially differentiate between S135 and L135 in the FcγRs indicated that polar residues are favorable for serine over leucine, with preferred residues being D, E, T, S, H, N and Q. Thus, the above analysis suggests that position 330* in variants v19544 and v27293 interacts with position S135 in FcγRIIb.

TABLE 6.6
Effect of Different Mutations at Position 330*

| | | | | | | | | IIb/IIaR | |
|---|---|---|---|---|---|---|---|---|---|
| | | | | IIb-Fold | | IIaR-Fold | | Selectivity² | |
| | | FcyRIIb | FcyRIIaR | vs. | vs. | vs. | vs. | vs. | vs. |
| Variant # | Mutation1 | KD/M | KD/M | WT | Control | WT | Control | WT | Control |
| 16463 | WT | 1.40E−06 | 3.10E−07 | 1 | | 1 | | 1 | |
| 27293 | Strat1 control³ | 3.50E−09 | 8.80E−09 | 415 | 1 | 35 | 1 | 11.8 | 1 |
| 26505 | B_G330*P_strat1 | 4.60E−08 | 1.10E−07 | 31.6 | 0.1 | 3 | 0.1 | 11.5 | 1 |

Hydrophobic Mutations

| | | | | | | | | | |
|---|---|---|---|---|---|---|---|---|---|
| 26489 | B_G330*V_strat1 | 2.20E−10 | 3.50E−10 | 6539 | 15.7 | 890 | 25.3 | 7.3 | 0.6 |
| 26488 | B_G330*A_strat1 | 5.10E−10 | 2.40E−09 | 2829 | 6.8 | 128 | 3.6 | 22.1 | 1.9 |
| 26490 | B_G330*L_strat1 | 8.90E−11 | 2.80E−10 | 16153 | 38.9 | 1104 | 31.4 | 14.6 | 1.2 |
| 26491 | B_G330*I_strat1 | 5.80E−11 | 2.00E−10 | 24999 | 60.2 | 1530 | 43.5 | 16.3 | 1.4 |
| 26492 | B_G330*M_strat1 | 5.40E−10 | 7.50E−10 | 2693 | 6.5 | 414 | 11.8 | 6.5 | 0.6 |
| 26493 | B_G330*F_strat1 | 1.60E−09 | 1.80E−09 | 910 | 2.2 | 168 | 4.8 | 5.4 | 0.5 |
| 26494 | B_G330*W_strat1 | 2.40E−09 | 2.80E−09 | 610 | 1.5 | 112 | 3.2 | 5.5 | 0.5 |
| 26495 | B_G330*Y_strat1 | 1.70E−09 | 3.10E−09 | 861 | 2.1 | 100 | 2.8 | 8.6 | 0.7 |

Hydrophilic Mutations

| | | | | | | | | | |
|---|---|---|---|---|---|---|---|---|---|
| 26496 | B_G330*T_strat1 | 6.70E−10 | 2.10E−09 | 2166 | 5.2 | 149 | 4.2 | 14.5 | 1.2 |
| 26497 | B_G330*S_strat1 | 5.10E−10 | 1.60E−09 | 2825 | 6.8 | 193 | 5.5 | 14.7 | 1.2 |
| 26498 | B_G330*Q_strat1 | 2.10E−10 | 5.10E−10 | 6945 | 16.7 | 605 | 17.2 | 11.5 | 1 |
| 26499 | B_G330*N_strat1 | 1.10E−09 | 2.90E−09 | 1334 | 3.2 | 108 | 3.1 | 12.4 | 1 |
| 26500 | B_G330*D_strat1 | 5.10E−09 | 3.20E−08 | 282 | 0.7 | 10 | 0.3 | 29.4 | 2.5 |
| 26501 | B_G330*E_strat1 | 1.70E−09 | 6.90E−09 | 857 | 2.1 | 45 | 1.3 | 19.1 | 1.6 |
| 26502 | B_G330*R_strat1 | 7.80E−09 | 3.40E−08 | 185 | 0.4 | 9 | 0.3 | 20.5 | 1.7 |
| 26503 | B_G330*K_strat1 | 1.40E−09 | 1.20E−08 | 1025 | 2.5 | 27 | 0.8 | 38.7 | 3.3 |
| 26504 | B_G330*H_strat1 | 9.20E−10 | 5.10E−09 | 1571 | 3.8 | 61 | 1.7 | 25.8 | 2.2 |

¹Mutation notation is in the following format: B_G330*P_strat1, where “B” indicates chain B of the Fc, “330*P” indicates the position and replacement amino acid of the mutation, and “strat1” indicates the parental CH2 mutations.
²Selectivity is defined as IIb-Fold/IIaR-Fold
³Strat1 = Strategy 1 Launching Module v27293

### Selectivity—Position 329*

As shown in FIG. 10(B), mutations at position 329* produced the greatest improvement in FcγRIIb binding selectivity. Position 329* is also within the loop inserted into variant v19544 and is proximal to position 135 in the receptor.

Analysis of the different mutations made at position 329* as shown in Table 6.7 reveals the following trends:
i) A wide range of mutations at this position improved FcγRIIb selectivity, although with very different levels of affinity.
ii) Aliphatic hydrophobic mutations showed the greatest improvement in FcγRIIb selectivity and affinity.
iii) Small hydrophobic mutations likely induce a conformational change that allows a selective binding mode to S135 in FcγRIIb.
iv) Aromatic hydrophobic mutations provided a good improvement in FcγRIIb selectivity, but with a much-decreased affinity.
v) Neutral and charged hydrophilic mutations improved FcγRIIb selectivity slightly, at the cost of affinity.
vi) The exceptions to point v) were glutamate (E) and glutamine (Q), which did not improve FcγRIIb selectivity.

The data suggests that Asp at position 329* in variants v19544 and v27293 interacts with R134 in the receptor as this residue is shared by both the FcγRIIb and FcγRIIaR receptors.

TABLE 6.7
Effect of Different Mutations at Position 329*

| | | | | | | | | IIb/IIaR | |
|---|---|---|---|---|---|---|---|---|---|
| | | | | IIb-Fold | | IIaR-Fold | | Selectivity² | |
| | | FcγRIIb | FcγRIIaR | vs. | vs. | vs. | vs. | vs. | vs. |
| Variant # | Mutation¹ | KD/M | KD/M | WT | Control | WT | Control | WT | Control |
| 16463 | WT | 1.40E−06 | 3.10E−07 | 1 | | 1 | | 1 | |
| 27293 | Strat1 control³ | 3.50E−09 | 8.80E−09 | 415 | 1 | 35.2 | 1 | 11.8 | 1 |

Hydrophobic Aliphatic Mutations

| | | | | | | | | | |
|---|---|---|---|---|---|---|---|---|---|
| 26470 | B_D329*G_strat1 | 1.40E−09 | 5.30E−09 | 1039 | 2.5 | 57.8 | 1.6 | 18 | 1.5 |
| 26471 | B_D329*A_strat1 | 1.50E−08 | 6.10E−08 | 93.4 | 0.2 | 5 | 0.1 | 18.5 | 1.6 |
| 26487 | B_D329*P_strat1 | 4.50E−09 | 1.60E−08 | 323 | 0.8 | 18.8 | 0.5 | 17.2 | 1.5 |
| 26472 | B_D329*V_strat1 | 2.00E−08 | 1.30E−07 | 72.8 | 0.2 | 2.4 | 0.1 | 30.5 | 2.6 |
| 26473 | B_D329*L_strat1 | 4.10E−09 | 3.30E−08 | 350 | 0.8 | 9.5 | 0.3 | 37 | 3.1 |
| 26474 | B_D329*I_strat1 | 5.60E−09 | 6.90E−08 | 259 | 0.6 | 4.5 | 0.1 | 57.7 | 4.9 |
| 26475 | B_D329*M_strat1 | 3.40E−09 | 1.40E−08 | 422 | 1 | 22 | 0.6 | 19.2 | 1.6 |

Hydrophobic Mutations

| | | | | | | | | | |
|---|---|---|---|---|---|---|---|---|---|
| 26476 | B_D329*F_strat1 | 3.20E−08 | 2.20E−07 | 45.3 | 0.1 | 1.4 | 0 | 31.7 | 2.7 |
| 26477 | B_D329*W_strat1 | 2.60E−08 | 1.30E−07 | 55.6 | 0.1 | 2.3 | 0.1 | 24 | 2 |
| 26478 | B_D329*Y_strat1 | 5.80E−08 | 3.90E−07 | 24.7 | 0.1 | 0.8 | 0 | 31.2 | 2.6 |

Hydrophilic Neutral Mutations

| | | | | | | | | | |
|---|---|---|---|---|---|---|---|---|---|
| 26479 | B_D329*T_strat1 | 1.90E−08 | 9.90E−08 | 77.4 | 0.2 | 3.1 | 0.1 | 24.8 | 2.1 |
| 26480 | B_D329*S_strat1 | 1.80E−08 | 7.40E−08 | 79.9 | 0.2 | 4.2 | 0.1 | 19.1 | 1.6 |
| 26481 | B_D329*Q_strat1 | 4.20E−09 | 1.40E−08 | 348 | 0.8 | 22.9 | 0.6 | 15.2 | 1.3 |
| 26482 | B_D329*N_strat1 | 2.00E−08 | 9.00E−08 | 71.9 | 0.2 | 3.4 | 0.1 | 21.1 | 1.8 |

Hydrophilic Charged Mutations

| | | | | | | | | | |
|---|---|---|---|---|---|---|---|---|---|
| 26483 | B_D329*E_strat1 | 1.60E−09 | 4.30E−09 | 914 | 2.2 | 71.7 | 2 | 12.7 | 1.1 |
| 26484 | B_D329*R_strat1 | 3.70E−08 | 2.50E−07 | 39.2 | 0.1 | 1.2 | 0 | 31.5 | 2.7 |
| 26485 | B_D329*K_strat1 | 3.30E−08 | 2.00E−07 | 44.3 | 0.1 | 1.5 | 0 | 29.4 | 2.5 |
| 26486 | B_D329*H_strat1 | 4.70E−08 | 1.90E−07 | 30.7 | 0.1 | 1.7 | 0 | 18.5 | 1.6 |

^{1, 2, 3}See footnotes to Table 6.6

### Strategy 2

Strategy 2 involved conducting a systematic 1× scan of residues in the environment of the Fc/FcγR interaction to identify those that could potentially further improve the selectivity of the original v19585 design. Residues that were deemed to be close to the interface of the FcγR were selected for screening and mutations compatible with the relevant secondary structure elements were selected for testing. Specifically, residues in loops were mutated to all possible amino acids except cysteine (18 amino acids) and residues in beta sheet positions were mutated with compatible residues (7 amino acids) as shown in Table 6.8. The total number of variants constructed was 542.

TABLE 6.8
Mutations Tested under Strategy 2¹

| | | Parent | | No. of |
|---|---|---|---|---|
| | | Amino | | Substi- |
| Chain | Position | Acid | Amino Acid Substitutions | tutions |
| A | 234 | Leu | G, A, V, I, F, W, Y, T, S, Q, N, D, | 17 |
| | | | E, R, K, H, P | |
| A | 235 | Leu | G, A, V, I, F, W, Y, T, S, Q, N, D, | 17 |
| | | | E, R, K, H, P | |
| A | 236 | Gly | A, V, L, I, F, W, Y, T, S, Q, N, D, | 17 |
| | | | E, R, K, H, P | |
| A | 237 | Gly | A, V, L, I, F, W, Y, T, S, Q, N, D, | 17 |
| | | | E, R, K, H, P | |
| A | 239 | Ser | G, A, V, L, I, F, W, Y, T, Q, N, D, | 17 |
| | | | E, R, K, H, P | |
| A | 264 | Val | A, L, I, M, F, T | 7 |
| A | 266 | Val | A, L, I, M, F, T | 7 |
| A | 267 | Ser | G, A, V, L, I, F, W, Y, T, Q, N, R, | 15 |
| | | | K, H, P | |
| A | 268 | Asp | G, A, V, L, I, F, W, Y, T, S, Q, N, | 17 |
| | | | E, R, K, H, P | |
| A | 269 | Glu | G, A, V, L, I, F, W, Y, T, S, Q, N, | 17 |
| | | | D, R, K, H, P | |
| A | 270 | Asp | G, A, V, L, I, F, W, Y, T, S, Q, N, | 17 |
| | | | E, R, K, H, P | |
| A | 271 | Pro | G, A, V, L, I, F, W, Y, T, S, Q, N, | 17 |
| | | | D, E, R, K, H | |
| A | 272 | Glu | G, A, V, L, I, F, W, Y, T, S, Q, N, | 17 |
| | | | D, R, K, H, P | |
| A | 273 | Val | A, L, I, M, F, T | 7 |
| A | 323 | Val | A, L, I, M, F, T | 7 |
| A | 325 | Asn | G, A, V, L, I, F, W, Y, T, S, Q, D, | 17 |
| | | | E, R, K, H, P | |
| A | 326 | Lys | G, A, V, L, I, F, W, Y, T, S, Q, N, | 17 |
| | | | D, E, R, H, P | |
| A | 327 | Ala | G, V, L, I, F, W, Y, T, S, Q, N, D, | 17 |
| | | | E, R, K, H, P | |
| A | 329 | Pro | G, A, V, L, I, F, W, Y, T, S, Q, N, | 17 |
| | | | D, E, R, K, H | |
| A | 330 | Ala | G, V, L, I, F, W, Y, T, S, Q, N, D, | 17 |
| | | | E, R, K, H, P | |
| A | 331 | Pro | G, A, V, L, I, F, W, Y, T, S, Q, N, | 17 |
| | | | D, E, R, K, H | |
| A | 332 | Ile | A, V, L, M, F, T | 7 |
| B | 234 | Leu | G, A, V, I, F, W, Y, T, S, Q, N, D, | 17 |
| | | | E, R, K, H, P | |
| B | 235 | Leu | G, A, V, I, F, W, Y, T, S, Q, N, D, | 17 |
| | | | E, R, K, H, P | |
| B | 236 | Gly | A, V, L, I, F, W, Y, T, S, Q, N, D, | 17 |
| | | | E, R, K, H, P | |
| B | 237 | Gly | A, V, L, I, F, W, Y, T, S, Q, N, D, | 17 |
| | | | E, R, K, H, P | |
| B | 239 | Ser | G, A, V, L, I, F, W, Y, T, Q, N, D, | 17 |
| | | | E, R, K, H, P | |
| B | 240 | Val | A, L, I, M, F, T | 7 |
| B | 263 | Val | A, L, I, M, F, T | 7 |
| B | 264 | Val | A, L, I, M, F, T | 7 |
| B | 266 | Val | A, L, I, M, F, T | 7 |
| B | 267 | Ser | G, A, V, L, I, F, W, Y, T, Q, N, R, | 15 |
| | | | K, H, P | |
| B | 268 | Asp | G, A, V, L, I, F, W, Y, T, S, Q, N, | 17 |
| | | | E, R, K, H, P | |
| B | 269 | Glu | G, A, V, L, I, F, W, Y, T, S, Q, N, | 17 |
| | | | D, R, K, H, P | |
| B | 270 | Asp | G, A, V, L, I, F, W, Y, T, S, Q, N, | 17 |
| | | | E, R, K, H, P | |
| B | 271 | Pro | G, A, V, L, I, F, W, Y, T, S, Q, N, | 17 |
| | | | D, E, R, K, H | |
| B | 272 | Glu | G, A, V, L, I, F, W, Y, T, S, Q, N, | 17 |
| | | | H, P | |
| B | 273 | Val | D, R, K, A, L, I, M, F, T | 7 |

¹Based on Launching Module 2 (v27294): A: L234F_G236N_H268Q_A327G_A330K_P331S/B: G236D_S239D_V266L_S267A_H268D

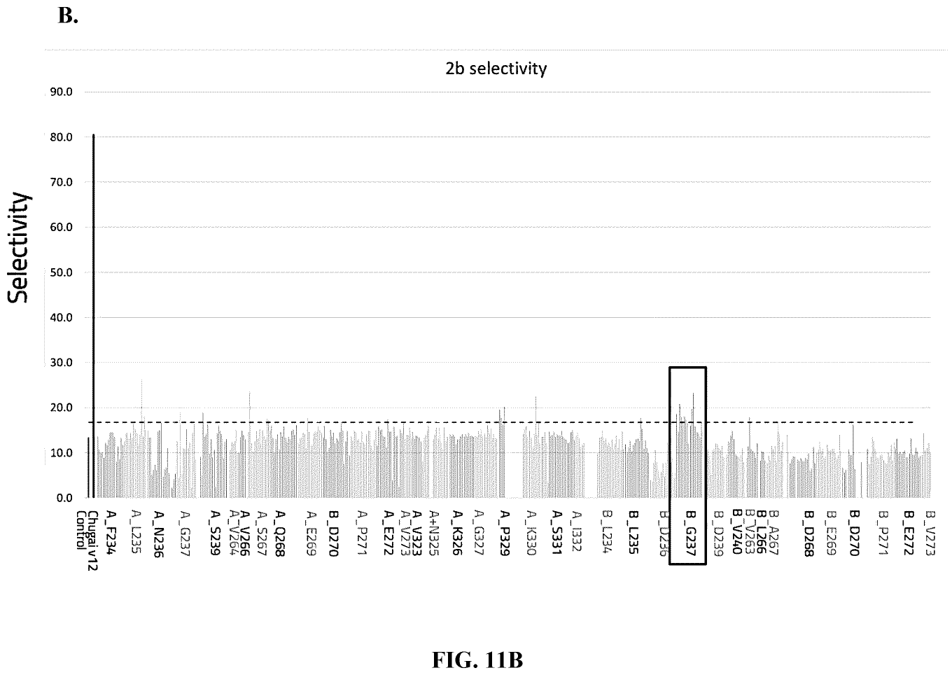

Variants were constructed in a one-armed antibody scaffold (Scaffold 2) and tested for FcγR binding by SPR as described in the General Methods (Protocol 2). The results are summarized in FIGS. 11A & B.

As shown in FIG. 11(A), mutations at position 237 on either chain of the Fc resulted in the greatest improvement in FcγRIIb affinity. FIG. 11(B) shows that only modest improvements were made in FcγRIIb selectivity by Strategy 2 mutations. Mutations at position 237 in chain B showed the best improvement in FcγRIIb selectivity, with some individual mutations in other positions also showing some improvement in FcγRIIb selectivity.

### Strategy 3

For strategy 3, variant v27362 was used as the launching module and combined with various loop templates from Example 2 in place of Template 1. In Example 2, mutations were tested in the template at the anchor positions and loop tips to identify templates with improved selectivity. For Strategy 3, loop templates with a selectivity greater than 3-fold from Example 2, as well as new templates comprising combinations of anchor and tip mutations that could potentially improve selectivity were tested in combination with the mutations of variant v27362. The tested variants are summarized in Table 6.9A & B.

TABLE 6.9A
Top Selective Loop Variants (Selectivity >3-Fold) Tested in Strategy 3

| Variant | | IIb - | IIaR- | IIb/IIaR |
|---|---|---|---|---|
| # | Description¹ | Fold² | Fold² | Selectivity³ |
| 20771 | Template_66\|D327*D_Q328*D_N329*E_Q330*D | 350 | 84.5 | 4.1 |
| 20688 | Template_66\|D327*D_Q328*P_N329*D_Q330*Q | 311 | 79.3 | 3.9 |
| 20972 | Template_1\|T326*H_W327*W_F328*S_D329*D | 94.3 | 24.9 | 3.8 |
| 20761 | Template_66\|D327*D_Q328*E_N329*D_Q330*D | 372 | 103.1 | 3.6 |
| 20976 | Template_1\|T326*H_W327*W_F328*E_D329*D | 92.5 | 25.7 | 3.6 |
| 20451 | Template_19\|V325*A | 11.4 | 3.2 | 3.6 |
| 20975 | Template_1\|T326*H_W327*W_F328*E_D329*G | 282 | 79.4 | 3.5 |
| 20965 | Template_1\|T326*H_W327*W_F328*Q_D329*G | 133 | 37.6 | 3.5 |
| 20964 | Template_1\|T326*H_W327*W_F328*F_D329*D | 152 | 43.9 | 3.5 |
| 20758 | Template_66\|D327*D_Q328*E_N329*E_Q330*Q | 262 | 76.4 | 3.4 |
| 21008 | Template_1\|T326*T_W327*W_F328*S_D329*D | 62.9 | 18.3 | 3.4 |
| 20724 | Template_66\|D327*D_Q328*H_N329*D_Q330*Q | 310 | 90.6 | 3.4 |
| 20968 | Template_1\|T326*H_W327*W_F328*N_D329*D | 67.1 | 20 | 3.4 |
| 21012 | Template_1\|T326*T_W327*W_F328*E_D329*D | 78.2 | 23.3 | 3.4 |
| 20733 | Template_66\|D327*D_Q328*S_N329*T_Q330*D | 420 | 125.7 | 3.3 |
| 20713 | Template_66\|D327*D_Q328*N_N329*D_Q330*D | 492 | 147.4 | 3.3 |
| 20749 | Template_66\|D327*D_Q328*T_N329*D_Q330*D | 452 | 140.2 | 3.2 |
| 20872 | Template_7\|E328*E_E329*N | 110 | 34.3 | 3.2 |
| 20674 | Template_66\|D327*N_Q328*D_N329*E_Q330*Q | 188 | 59 | 3.2 |
| 20732 | Template_66\|D327*D_Q328*S_N329*T_Q330*Q | 289 | 90.7 | 3.2 |
| 20966 | Template_1\|T326*H_W327*W_F328*Q_D329*D | 45.7 | 14.5 | 3.2 |
| 20384 | Template_66\|1332Q | 180 | 57.3 | 3.1 |
| 20742 | Template_66\|D327*D_Q328*T_N329*S_Q330*Q | 308 | 98 | 3.1 |
| 21001 | Template_1\|T326*T_W327*W_F328*Q_D329*G | 90.7 | 29.1 | 3.1 |
| 21007 | Template_1\|T326*T_W327*W_F328*S_D329*G | 145 | 47.5 | 3.1 |
| 20505 | Template_1\|S325*A | 29.8 | 9.8 | 3.1 |
| 20639 | Template_66\|D327*N_Q328*H_N329*N_Q330*D | 177 | 58 | 3.1 |
| 21000 | Template_1\|T326*T_W327*W_F328*F_D329*D | 117 | 38.3 | 3 |
| 20974 | Template_1\|T326*H_W327*W_F328*T_D329*D | 83.1 | 27.8 | 3 |
| 20978 | Template_1\|T326*H_W327*W_F328*D_D329*D | 72.7 | 24.4 | 3 |
| 20864 | Template_7\|E328*T_E329*N | 96.4 | 32.5 | 3 |
| 20970 | Template_1\|T326*H_W327*W_F328*H_D329*D | 110 | 37.1 | 3 |
| 20766 | Template_66\|D327*D_Q328*D_N329*S_Q330*Q | 353 | 119.7 | 3 |

¹Nomenclature used to describe the loops and mutations is based on: Template_Y|X327*Z, where Y indicates the loop template number, X is the amino acid found at the listed position in the parental loop sequence, and Z is the amino acid mutation.
²Fold change in affinity over wild-type
³Selectivity is defined as IIb-Fold/IIaR-Fold

TABLE 6.9B
New Loop Templates Comprising Combinations of Anchor and Tip Mutations Tested in Strategy 3

| | | | | | | No. of |
|---|---|---|---|---|---|---|
| Starting | Starting | Additional Mutations | | | | Variants |
| Variant # | Loop¹ | 1X | 1X | 1X | 2X | Tested |
| 20972 | Template_1\|T326*H_W327*W_F328*S_D329*D | S325*A | A331*BN | | S325*A_A331*BN | 3 |
| 20976 | Template_1\|T326*H_W327*W_F328*E_D329*D | S325*A | A331*BN | | S325*A_A331*BN | 3 |
| 20975 | Template_1\|T326*H_W327*W_F328*E_D329*G | S325*A | A331*BN | | S325*A_A331*BN | 3 |
| 20965 | Template_1\|T326*H_W327*W_F328*Q_D329*G | S325*A | A331*BN | | S325*A_A331*BN | 3 |
| 20964 | Template_1\|T326*H_W327*W_F328*F_D329*D | S325*A | A331*BN | | S325*A_A331*BN | 3 |
| 21008 | Template_1\|T326*T_W327*W_F328*S_D329*D | S325*A | A331*BN | | S325*A_A331*BN | 3 |
| 20771 | Template_66\|D327*D_Q328*D_N329*E_Q330*D | I332Q | I332W | D325*A | I332Q_D325*A | 4 |
| 20688 | Template_66\|D327*D_Q328*P_N329*D_Q330*Q | I332Q | I332W | D325*A | I332Q_D325*A | 4 |
| 20761 | Template_66\|D327*D_Q328*E_N329*D_Q330*D | I332Q | I332W | D325*A | I332Q_D325*A | 4 |
| 20758 | Template_66\|D327*D_Q328*E_N329*E_Q330*Q | I332Q | I332W | D325*A | I332Q_D325*A | 4 |
| 20724 | Template_66\|D327*D_Q328*H_N329*D_Q330*Q | I332Q | I332W | D325*A | I332Q_D325*A | 4 |
| 20733 | Template_66\|D327*D_Q328*S_N329*T_Q330*D | I332Q | I332W | D325*A | I332Q_D325*A | 4 |
| 20872 | Template_7\|E328*E_E329*N | A331*BV | A331*BY | G325*F | A331*BV_G325*F | 4 |
| 20864 | Template_7\|E328*T_E329*N | A331*BV | A331*BY | G325*F | A331*BV_G325*F | 4 |
| 20846 | Template_7\|E328*H_E329*R | A331*BV | A331*BY | G325*F | A331*BV_G325*F | 4 |
| 20834 | Template_7\|E328*Q_E329*S | A331*BV | A331*BY | G325*F | A331*BV_G325*F | 4 |
| 20576 | Template_151\|E328*H_E329*N | Y331*BI | R331*S | Y331*BQ | R331*S_Y331*BI | 4 |
| 20602 | Template_151\|E328*E_E329*D | Y331*BI | R331*S | Y331*BQ | R331*S_Y331*BI | 4 |

¹Nomenclature used to describe the loops and mutations is based on: Template_Y|X327*Z, where Y indicates the loop template number, X is the amino acid found at the listed position in the parental loop sequence, and Z is the amino acid mutation.

Variants were constructed in a one-armed antibody scaffold (Scaffold 2) and tested for FcγR binding by SPR as described in the General Methods (Protocol 2).

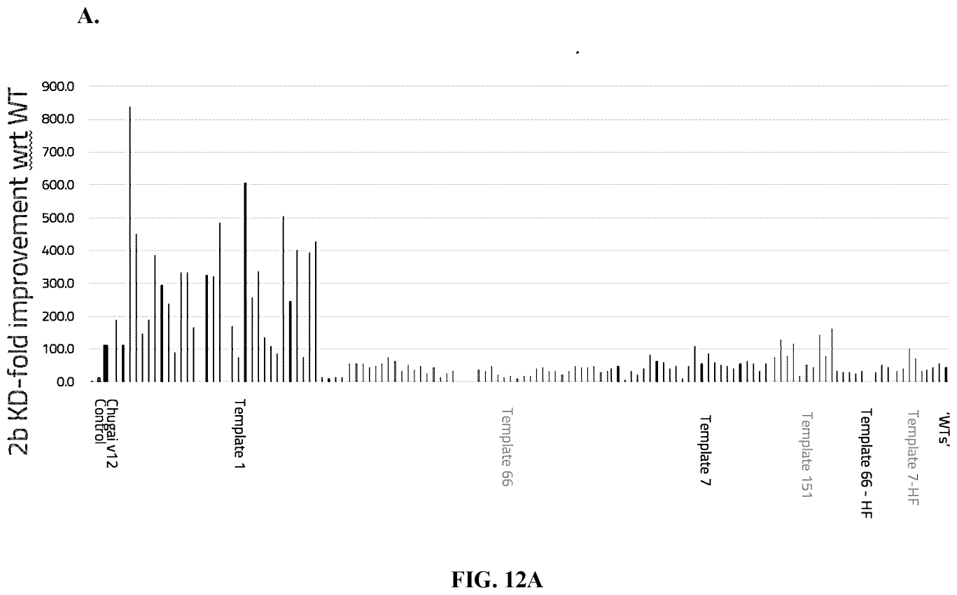

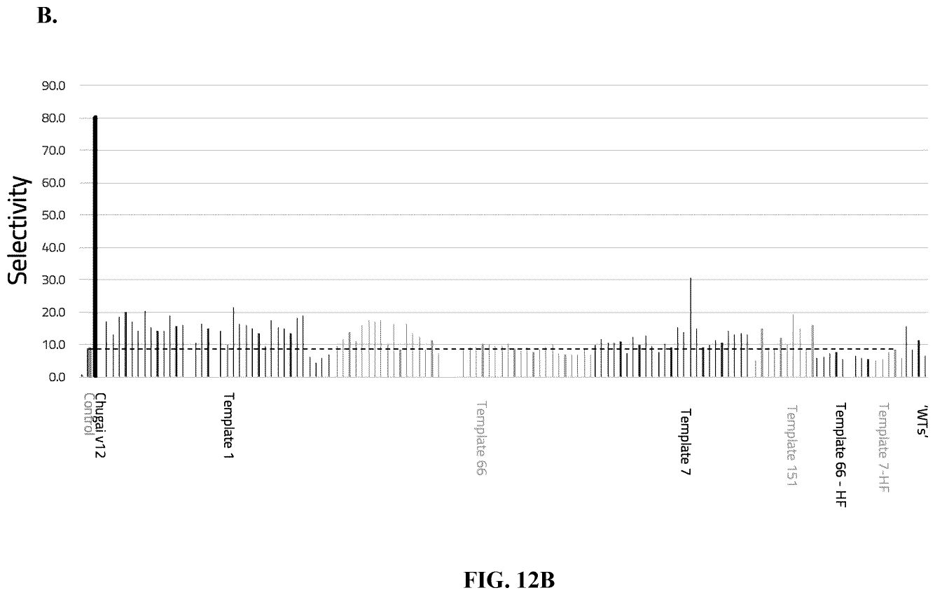

The results are summarized in FIGS. 12A & B. Template 1-based variants showed the largest improvement in FcγRIIb affinity (FIG. 12(A)), as well as yielding the most variants with improved FcγRIIb selectivity (FIG. 12(B)). Template 66 also yielded a number of variants with improved FcγRIIb selectivity, and Template 7 yielded one variant with the highest FcγRIIb selectivity of all the Strategy 3 variants tested (FIG. 12(B)). This Template 7 variant comprised the mutations E328*H_E329*R_A331*BY (loop sequence: GLDHRGKGYV [SEQ ID NO: 15]).

### Strategy 4

Longer loop replacement templates were analyzed using a similar procedure to that detailed in Example 2. The longer loops have the potential to produce stronger interactions between the loop and position S135 in FcγRIIb. Table 6.10 lists the criteria that were used to rank the loops.

TABLE 6.10
Selection Criteria for Longer Loops

| Criterion | Desirable Property |
|---|---|
| Root-mean-square deviation (RMSD) | Ideal loops should show compatibility between |
| of the energy-minimized grafted loop | donating and accepting environments, so loops should |
| with respect to the donating structure | show similar conformations when energy minimized |
| Tolerability to mutations | Loops of similar conformation but different sequences |
| | were inspected in the PDB. Ideal loops should maintain |
| | internal conformation when mutated at residues that |
| | could interact specifically with FcγRIIb |
| Low crystal contacts | In the donating structure, the loops should not be |
| | surrounded by crystal contacts that could be stabilizing |
| | an artificial conformation |
| Contacts with S135 in FcγRIIb | In silico models should show good interaction potential |
| | with S135 to drive selectivity of Fc binding |

Based on the criteria listed in Table 6.10, the following loops were selected for further analysis.

TABLE 6.11
Sequences of Longer Loops

| Loop ID | 325* | 326* | 327* | 328* | 329* | 330* | 331* | 331*A | 331*B | 331*C | 331*D | 331*E | 331*F | 331*G | SEQ ID NO |
|---|---|---|---|---|---|---|---|---|---|---|---|---|---|---|---|
| 13_3 | V | L | D | D | P | S | R | E | N | E | A | D | L | | 16 |
| 12_14 | N | F | T | P | K | A | K | L | G | F | E | I | | | 17 |
| 14_0 | Q | V | H | E | D | A | T | K | P | Y | G | L | S | L | 18 |
| 11_14 | A | P | Q | I | N | P | H | S | P | K | F | | | | 19 |
| 19 | V | T | W | E | D | G | K | S | E | R | | | | | 20 |

Additional mutations were made to the sequences of the selected loops in order to remove hydrophobic residues and/or to improve the anchor points when the loops were grafted onto the Fc chain B. Specifically, in silico modelling indicated that in many cases, the grafted loops formed a hydrophobic anchor that created a cavity. Positions 266, 273 and 325* were identified as the most promising positions to introduce mutations to minimize or remove this cavity. A 1× scan was carried out at these positions for all loops, as well as combinatorial testing (2× and 3×) for loops 13_3 and 12_14. In addition, for those positions identified in silico as the most likely to interact with position 5135 on the receptor, a combinatorial library was constructed for all loops.

These additional modifications are summarized in Tables 6.12, 6.13 and 6.14. A total of 489 variants were tested.

TABLE 6.12
Mutations to Remove Exposed Hydrophobic Residues

| Loop | Exposed Residues and Mutations¹ | | | | | | |
|---|---|---|---|---|---|---|---|
| ID | 326* | 331*C | 331*D | 331*E | 331*F | 331*G | 332 |

| | | | | | | |
|---|---|---|---|---|---|---|
| 13_3 | L → T | | A(WT)² | | L → T | |
| 12_14 | F → T | F → T | | I → T | | |
| 14_0 | V → T | Y → T | | L → T | | L → T |
| 11_14 | P → T | | F → T | | | |

¹Designations used are as follows: X → Y, where X is the residue found in the parental loop and Y is the mutated residue.
²Exposed hydrophobic residue, not mutated.

TABLE 6.13
Mutations to Improve Anchor Points¹

| | | Mutations² | | | No. of |
|---|---|---|---|---|---|
| | Loop ID | 266 | 273 | 325* | Variants |
| 1X scan | 13_3 | V → I, L or F | V → L, I or F | V → I, L or F | 9 |
| | 12_14 | V → I, L or F | V → L, I or F | N → D, V, I, L or F | 9 |
| | 14_0 | V → I, L or F | V → L, I or F | Q → V, I, L or F | 9 |
| | 11_14 | V → I, L or F | V → L, I or F | Q → V, I, L or F | 9 |
| Combinatorial | 13_3 | V → I, L or F | | V → I, L or F | 9 |
| 2X scan | 12_14 | V → I, L or F | | N → I, L or F | 9 |
| Combinatorial | 13_3 | V → I, L or F | V → I, L or F | V → I, L or F | 27 |
| 3X scan | | | | | |

¹These mutations were carried out in loop variants in which exposed hydrophobic residues had been mutated as shown in Table 6.12
²Designations used are as follows: X → Y, where X is the residue found in the parental loop and Y is the mutated residue.

TABLE 6.14
Combinatorial Library of Mutations¹

| Loop | Mutations in Anchor Residues | | | | | | | Mutations in Exposed Residues | | | | | | |
|---|---|---|---|---|---|---|---|---|---|---|---|---|---|---|
| ID | 266 | 273 | 325* | 326* | 328* | 329* | 330* | 331* | 331*A | 331*C | 331*D | 331*E | 331*F | 331*G |
| 13_3 | V, I | V | V, I | T | | | | D, E, | D, E, | | A | | T | |
| | | | | | | | | S, H, | S, H, | | | | | |
| | | | | | | | | N, | N | | | | | |
| | | | | | | | | R(WT) | | | | | | |
| 12_14 | V, I | V | I | T | | D, E, | D, E, | | | T | | T | | |
| | | | | | | N, S, | N, S, | | | | | | | |
| | | | | | | H | H | | | | | | | |
| | | | | | | K(WT) | A(WT) | | | | | | | |
| 14_0 | | | | T | | D, E, | D, E, | D, E, | | T | | T | | T |
| | | | | | | N, S | N, S, | N, S, | | | | | | |
| | | | | | | | A(WT) | T(WT) | | | | | | |
| 11_14 | | | | T | | D, E, | | D, E, | | | T | | | |
| | | | | | | N, S, | | N, S, | | | | | | |
| | | | | | | H | | H | | | | | | |
| Template_ | | | A | | D, E, | D, E, | | | | | | | | |
| 19\|V325A | | | | | N, S, | N, S, | | | | | | | | |
| | | | | | H | H | | | | | | | | |

¹The designation X(WT) indicates that X is the residue in the parental loop

Variants were constructed in a one-armed antibody scaffold (Scaffold 2) and tested for FcγR binding by SPR as described in the General Methods (Protocol 2).

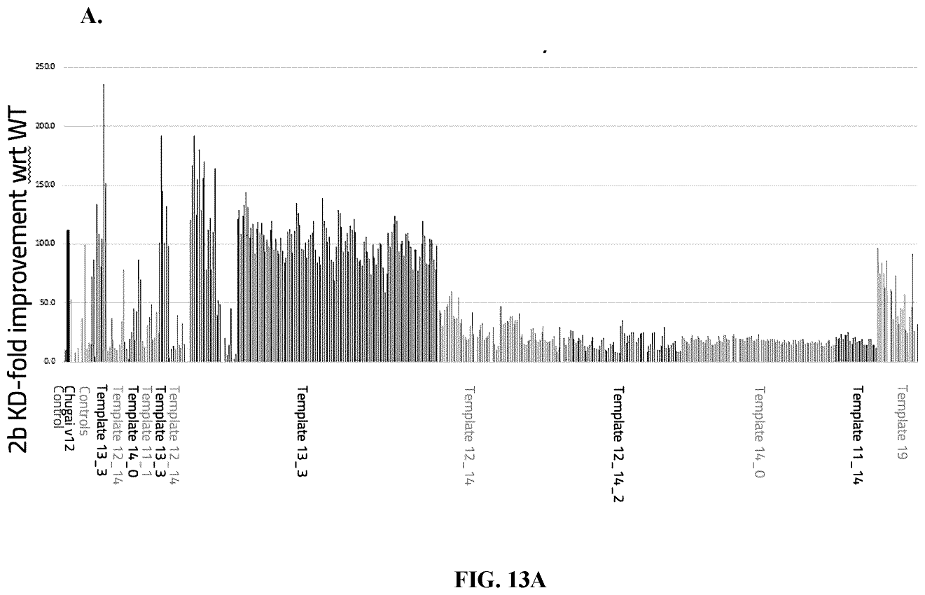

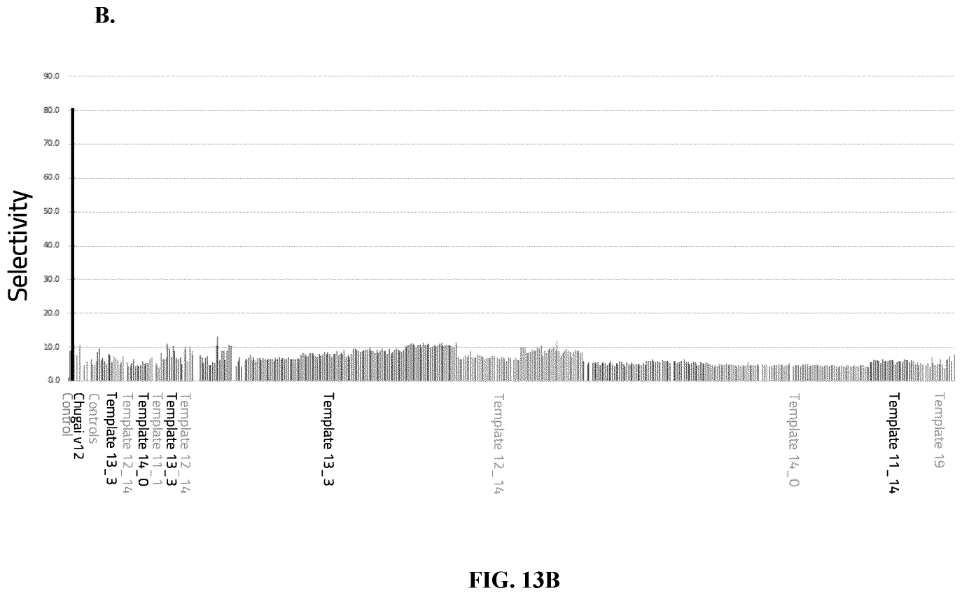

The results are summarized in FIG. 13. Variants based on Template 13_3 showed the greatest improvement in FcγRIIb affinity (FIG. 13(A)). None of the longer loop variants showed a significant improvement in FcγRIIb selectivity (FIG. 13(B)).

### Strategy 5

Strategy 5 involved combining the most promising stability mutations identified in Example 5 with Launching Modules 1 and 2 (v27293 and v27294, respectively). The variants generated by Strategy 5 were not expected to improve selectivity but rather were intended to improve stability of the Fc region. The stability mutations were introduced on both chains of the Fc.

The stability mutations tested were the following:
A287F+M428F
A287F+T250V
M428F+T250V
A287F+M428F+T250V
T250V+L309Q
L242C_I336C+V308I

Variants were constructed in a one-armed antibody scaffold (Scaffold 2) and tested for FcγR binding by SPR as described in the General Methods (Protocol 2). Thermal stability of the variants was measured by DSF as described in the General Methods.

The results are shown in Tables 6.15, 6.16 and 6.21. Overall, the stability mutations had minimal impact on FcγRIIb binding affinity or selectivity. One combination of stability mutations (A287F_M428F_T250V) disrupted binding in both Launching Module 1 and 2 (see variants v27314 and v27315 in Table 6.21) and one combination of stability mutations (L242C_I336C) disrupted binding in Launching Module 1 (see variant v27304 in Table 6.21). All stability mutations increased the thermal stability of both Launching Module 1 and 2.

TABLE 6.15
FcγR Binding of Strategy 5 Variants

| | | FcγR | FcγR | IIb-Fold | | IIaR-Fold | | Selectivity¹ | |
|---|---|---|---|---|---|---|---|---|---|
| | | IIb | IIaR | vs. | vs. | vs. | vs. | vs. | vs. |
| Variant # | Mutations | K_{D}/M | K_{D}/M | WT | Control | WT | Control | WT | Control |
| 16463 | WT | 1.4E−06 | 3.1E−07 | 1.0 | | 1.0 | | 1.0 | |
| 27293 | LM1²_control_A_G236N_ | 3.5E−09 | 8.8E−09 | 415 | 1.0 | 35.2 | 1.0 | 11.8 | 1.0 |
| | G237A_B_G236D_ | | | | | | | | |
| | G237F_S239D_S267V_ | | | | | | | | |
| | H268D_Template_1 | | | | | | | | |
| 27294 | LM2²_control_A_L234F_ | 1.6E−08 | 4.4E−08 | 92.9 | 1.0 | 7.0 | 1.0 | 13.4 | 1.0 |
| | G236N_H268Q_A327G_ | | | | | | | | |
| | A330K_P331S_ | | | | | | | | |
| | B_G236D_S239D_V266L_ | | | | | | | | |
| | S267A_H268D | | | | | | | | |
| 27296 | LM1_A287F_strat5 | 3.1E−09 | 1.1E−08 | 469 | 1.1 | 28.6 | 0.8 | 16.4 | 1.4 |
| 27297 | LM2_A287F_strat5 | 2.0E−08 | 6.2E−08 | 71.2 | 0.2 | 4.9 | 0.1 | 14.4 | 1.2 |
| 27298 | LM1_M428F_strat5 | 3.3E−09 | 1.2E−08 | 437 | 1.1 | 26.4 | 0.7 | 16.6 | 1.4 |
| 27300 | LM1_T250V_strat5 | 3.0E−09 | 1.1E−08 | 479 | 1.2 | 27.8 | 0.8 | 17.2 | 1.5 |
| 27301 | LM2_T250V_strat5 | 1.7E−08 | 5.6E−08 | 84.5 | 0.2 | 5.5 | 0.2 | 15.3 | 1.3 |
| 27302 | LM1_L309Q_strat5 | 2.8E−09 | 1.1E−08 | 512 | 1.2 | 28.4 | 0.8 | 18.1 | 1.5 |
| 27303 | LM2_L309Q_strat5 | 2.1E−08 | 6.0E−08 | 69 | 0.2 | 5.1 | 0.1 | 13.5 | 1.1 |
| 27305 | LM2_L242C_I336C_strat5 | 1.9E−08 | 6.6E−08 | 74.5 | 0.2 | 4.7 | 0.1 | 15.9 | 1.3 |
| 27306 | LM1_V308I_strat5 | 3.5E−09 | 1.6E−08 | 417 | 1.0 | 19.8 | 0.6 | 21.1 | 1.8 |
| 27307 | LM2_V308I_strat5 | 2.0E−08 | 5.9E−08 | 74.1 | 0.2 | 5.2 | 0.1 | 14.3 | 1.2 |
| 27308 | LM1_A287F_M428F_strat5 | 3.1E−09 | 1.1E−08 | 465 | 1.1 | 29.1 | 0.8 | 16.0 | 1.4 |
| 27309 | LM2_A287F_M428F_strat5 | 1.9E−08 | 5.8E−08 | 75.7 | 0.2 | 5.3 | 0.2 | 14.3 | 1.2 |
| 27310 | LM1_A287F_T250V_strat5 | 2.0E−09 | 8.8E−09 | 733 | 1.8 | 35.2 | 1.0 | 20.8 | 1.8 |
| 27311 | LM2_A287F_T250V_strat5 | 1.7E−08 | 5.9E−08 | 85.0 | 0.2 | 5.2 | 0.1 | 16.3 | 1.4 |
| 27312 | LM1_M428F_T250V_strat5 | 4.1E−09 | 1.4E−08 | 349 | 0.8 | 22.5 | 0.6 | 15.5 | 1.3 |
| 27313 | LM2_M428F_T250V_strat5 | 1.8E−08 | 5.3E−08 | 79.0 | 0.2 | 5.8 | 0.2 | 13.5 | 1.1 |
| 27316 | LM1_T250V_L309Q_strat5 | 3.9E−09 | 1.4E−08 | 373 | 0.9 | 22.2 | 0.6 | 16.8 | 1.4 |
| 27317 | LM2_T250V_L309Q_strat5 | 1.9E−08 | 5.6E−08 | 77.7 | 0.2 | 5.5 | 0.2 | 14.2 | 1.2 |
| 27318 | LM1_L242C_I336C_ | 3.6E−09 | 1.2E−08 | 403 | 1.0 | 24.9 | 0.7 | 16.2 | 1.4 |
| | V308I_strat5 | | | | | | | | |
| 27319 | LM2_L242C_I336C_ | 1.6E−08 | 4.3E−08 | 92.6 | 0.2 | 7.2 | 0.2 | 12.9 | 1.1 |
| | V308I_strat5 | | | | | | | | |

¹Selectivity is defined as IIb-Fold/IIaR-Fold
²LM1 = Launching Module 1; LM2 = Launching Module 2

TABLE 6.16
Stability of Strategy 5 Variants

| | | Tm/° | Δ² | | | Tm/° | Δ² | | Predicted |
|---|---|---|---|---|---|---|---|---|---|
| Variant # | Mutation¹ | C. | Tm | Variant # | Mutation | C. | Tm | Ave.³ | if additive |
| 16463 | WT | 69.0 | | | | | | | |
| 27293 | strat1_control_A_ | 59.0 | 0.0 | 27294 | strat2_control_A_ | 62 | 0.0 | | |
| | G236N_G237A_ | | | | L234F_G236N_ | | | | |
| | B_G236D_G237F_ | | | | H268Q_A327G_ | | | | |
| | S239D_S267V_ | | | | A330K_P331S_ | | | | |
| | H268D_Template1 | | | | B_G236D_S239D_ | | | | |
| | | | | | V266L_S267A_ | | | | |
| | | | | | H268D | | | | |
| 27296 | LM1_A287F_strat5 | 62.5 | 3.5 | 27297 | LM2_A287F_strat5 | 66 | 4.0 | 3.8 | |
| 27298 | LM1_M428F_strat5 | 61.0 | 2.0 | | | | | | |
| 27300 | LM1_T250V_strat5 | 64.5 | 5.5 | 27301 | LM2_T250V_strat5 | 67.5 | 5.5 | 5.5 | |
| 27302 | LM1_L309Q_strat5 | 61.0 | 2.0 | 27303 | LM2_L309Q_strat5 | 64.5 | 2.5 | 2.3 | |
| 27304 | LM1_L242C_I336C_strat5 | 60.0 | 1.0 | 27305 | LM2_L242C_I336C_strat5 | 62 | 0.0 | 0.5 | |
| 27306 | LM1_V308I_strat5 | 59.5 | 0.5 | 27307 | LM2_V308I_strat5 | 63 | 1.0 | 0.8 | |
| 27308 | LM1_A287F_M428F_strat5 | 65.5 | 6.5 | 27309 | LM2_A287F_M428F_strat5 | 69 | 7.0 | 6.8 | 5.3 |
| 27310 | LM1_A287F_T250V_strat5 | 68.0 | 9.0 | 27311 | LM2_A287F_T250V_strat5 | 71.5 | 9.5 | 9.3 | 9.3 |
| 27312 | LM1_M428F_T250V_strat5 | 67.5 | 8.5 | 27313 | LM2_M428F_T250V_strat5 | 60 | −2.0 | 3.3 | 9.3 |
| 27316 | LM1_T250V_L309Q_strat5 | 68.0 | 9.0 | 27317 | LM2_T250V_L309Q_strat5 | 70.5 | 8.5 | 8.8 | 7.8 |
| 27318 | LM1_L242C_I336C_ | 62.0 | 3.0 | 27319 | LM2_L242C_I336C_ | 62.5 | 0.5 | 1.8 | 1.3 |
| | V308I_strat5 | | | | V308I_strat5 | | | | |

¹LM1 = Launching Module 1; LM2 = Launching Module 2
²Change over parental variant
³Ave. = Average Δ Tm over LM1 and LM2 variants

The complete results for Strategies 1-5 are shown in Tables 6.17-6.21. The variants generated from the strategies outlined above showed a range of FcγRIIb selectivities and affinities. Selection of variants that met specified criteria for changes in FcγRIIb selectivity and/or affinity with respect to the parental control allowed for generation of a library of variants with a range of FcγRIIb-binding profiles.

The following Criteria were developed to define variants having useful FcγRIIb-binding profiles (“Control” in each case is the respective parental variant as noted in Tables 6.17-6.21):

Criteria A: “IIb Selectivity Fold wrt Control”>1.5 and “IIb-Fold wrt Control”>0.3.

Criteria B: “IIb Selectivity Fold wrt Control”>0.5 and “IIb-Fold wrt Control”>0.5.

Criteria C: “IIb Selectivity Fold wrt Control”≥1.0 and “IIb-Fold wrt Control” value≥0.3.

Criteria D: “Ib Selectivity Fold wrt Control”≥1.0 and a “IIb-Fold wrt Control”≥0.5.

Tables 6.22-6.24 list variants from each of Strategies 1-3 that met Criteria A. Tables 6.25-6.27 list variants from each of Strategies 1-3 that met Criteria B. Variants that met either Criteria A or Criteria B were considered successful. Variants that met Criteria C are a subset of variants that met Criteria A, and variants that met Criteria D are a subset of variants that met Criteria B.

Sequences for the loops comprised by Strategy 1 and Strategy 3 variants meeting Criteria A are shown in Table 3A, and sequences for the loops comprised by Strategy 1 and Strategy 3 variants meeting Criteria B are shown in Table 3B.

### Example 7: Combination of Top Mutations—Lead Variants Generation 2 (LVG2)

Chain A and chain B mutations from a select number of variants from Example 6 showing good FcγRIIb selectivity were combined as shown in Tables 7.1-7.4 below. Variants were constructed in a one-armed antibody scaffold (Scaffold 2) and tested for FcγR binding by SPR as described in the General Methods (Protocol 2). Thermal stability and aggregation propensity of the variants were measured by DSF and aSEC, respectively, as described in the General Methods.

TABLE 7.1
Combinations of Strategy 1 Mutations

| | Chain A | Chain B | IIb- | IIaR- | IIb/IIaR | aSEC | aSEC | ΔTm/° |
|---|---|---|---|---|---|---|---|---|
| Variant # | Mutations¹ | Mutations¹ | Fold² | Fold² | Selectivity³ | Monomer/% | Aggregates/% | C.⁴ |
| 29688 | A237D_strat1 | D329*I_strat1 | 98.4 | 1.2 | 79.2 | 93.7 | 2.4 | −14.5 |
| 29689 | L235F_strat1 | D329*I_strat1 | 550 | 6.3 | 87.5 | 92.4 | 2.7 | −10.5 |
| 29690 | S239Y_strat1 | D329*I_strat1 | 220 | 3.4 | 64.5 | 91.5 | 3.1 | −13.5 |
| 29691 | L234D_strat1 | D329*I_strat1 | 312 | 5.7 | 54.5 | 94.6 | 3 | −11 |
| 29692 | S239G_strat1 | D329*I_strat1 | 248 | 3.6 | 68.1 | 92.3 | 2.8 | −13 |
| 29693 | A237L_strat1 | D329*I_strat1 | 118 | 1.8 | 67.1 | 93.3 | 2.9 | −10.5 |
| 29694 | A237D_strat1 | G330*K_strat1 | 234 | 5.1 | 45.4 | 90 | 2.8 | −13.5 |
| 29695 | L235F_strat1 | G330*K_strat1 | 1533 | 33.3 | 46 | 90.3 | 3 | −9.5 |
| 29696 | S239Y_strat1 | G330*K_strat1 | 987 | 20.9 | 47.2 | 89.5 | 2.8 | −12.5 |
| 29697 | L234D_strat1 | G330*K_strat1 | 1036 | 24.7 | 42 | 93.6 | 2.6 | −10.5 |
| 29698 | S239G_strat1 | G330*K_strat1 | 1008 | 22.4 | 45 | 90.1 | 2.8 | −12 |
| 29699 | A237L_strat1 | G330*K_strat1 | 435 | 8.4 | 51.8 | 91.4 | 3 | −9.5 |
| 29700 | A237D_strat1 | I332L_strat1 | 182 | 6.1 | 30 | 96.6 | 2.1 | −16.5 |
| 29701 | L235F_strat1 | I332L_strat1 | 608 | 29.8 | 20.4 | 95.4 | 2.5 | −11.5 |
| 29702 | S239Y_strat1 | I332L_strat1 | 422 | 19.3 | 21.8 | 96.1 | 2.4 | −14.5 |
| 29703 | L234D_strat1 | I332L_strat1 | 449 | 21.2 | 21.1 | 95.6 | 3.3 | −12 |
| 29704 | S239G_strat1 | I332L_strat1 | 430 | 18.9 | 22.8 | 95.7 | 2.7 | −14.5 |
| 29705 | A237L_strat1 | I332L_strat1 | 196 | 8.5 | 23.1 | 96 | 2.6 | −11 |
| 29706 | strat1_control + | D329*I_strat1 | 128 | 2 | 63 | 92.3 | 3.4 | −10.5 |
| | E269K | | | | | | | |
| 29707 | strat1_control + | G330*K_strat1 | 535 | 13.2 | 40.6 | 90.7 | 3.4 | −9.5 |
| | E269K | | | | | | | |
| 29708 | strat1_control + | I332L_strat1 | 275 | 12.6 | 21.8 | 95.9 | 2.8 | −11.5 |
| | E269K | | | | | | | |
| 29709 | S239H_strat1 | D329*I_strat1 | 152 | 2.5 | 61.3 | 92.4 | 3 | −11 |
| 29710 | S239H_strat1 | G330*K_strat1 | 725 | 18 | 40.3 | 92.6 | 3.1 | −10.5 |
| 29711 | S239H_strat1 | I332L_strat1 | 350 | 15.8 | 22.2 | 96 | 2.3 | −12 |

¹Mutation notation is in the format “A237D_strat1,” where “A237D” indicates the mutation made with “A” representing the parental residue being replaced, “237” representing the position and “D” representing the replacement residue, and “strat1” specifies the parental CH2 mutations (i.e. those of Launching Module 1).
²Fold change in affinity over wild-type
³Selectivity is defined as IIb-Fold/IIaR-Fold
⁴Compared to wild-type

All Strategy 1 combination variants showed reduced binding to the FcγRIIaH receptors. As shown in Table 7.1, differences in FcγRIIb affinity values were observed across Strategy 1 combination variants, but the variants showed similar FcγRIIb selectivity. No significant aggregation of Strategy 1 combination variants was revealed by aSEC. All Strategy 1 combination variants showed a decrease in Tm of between about 10° C. and 15° C.

TABLE 7.2
Combinations of Strategy 2 Mutations

| | Chain A | Chain B | IIb- | IIaR- | IIb/IIaR | aSEC | aSEC | ΔTm/° |
|---|---|---|---|---|---|---|---|---|
| Variant # | Mutations¹ | Mutations¹ | Fold² | Fold² | Selectivity³ | Monomer/% | Aggregates/% | C.⁴ |
| 29712 | L235D_strat2 | G237D_strat2 | 182 | 6.2 | 29.6 | 89.8 | 4.3 | −12 |
| 29713 | S267A_strat2 | G237D_strat2 | 195 | 7.8 | 24.9 | 89.6 | 4.8 | −12 |
| 29714 | K330T_strat2 | G237D_strat2 | 185 | 7 | 26.3 | 90.3 | 4.4 | −12 |
| 29715 | P329I_strat2 | G237D_strat2 | 115 | 3 | 38.7 | 89.8 | 3.3 | −12 |
| 29716 | L235D_strat2 | G237L_strat2 | 179 | 7.2 | 24.9 | 90.2 | 5.2 | −9.5 |
| 29717 | S267A_strat2 | G237L_strat2 | 177 | 8.4 | 20.9 | 91.2 | 3.5 | −9 |
| 29718 | K330T_strat2 | G237L_strat2 | 205 | 8.9 | 23 | 91.4 | 3.8 | −9 |
| 29719 | P329I_strat2 | G237L_strat2 | 92.1 | 3.2 | 28.6 | 91.5 | 3.7 | −9 |
| 29720 | L235D_strat2 | D270Y_strat2 | 2 | NB⁵ | | 86.1 | 7.4 | −8 |
| 29721 | S267A_strat2 | D270Y_strat2 | 2.7 | NB | | 86.5 | 6.7 | −7.5 |
| 29722 | K330T_strat2 | D270Y_strat2 | 3.6 | NB | | 88.8 | 5.9 | −7.5 |
| 29723 | P329I_strat2 | D270Y_strat2 | NB | NB | | 85.6 | 7.1 | −8 |

¹⁻⁴See footnotes to Table 7.1. “Strat2” indicates the parental CH2 mutations are those of Launching Module 2.
⁵NB = no binding

As shown in Table 7.2, lower FcγRIIb selectivity was observed for Strategy 2 combination variants as compared to Strategy 1 combination variants, as expected. More aggregate species were generally observed for Strategy 2 combination variants than for Strategy 1 combination variants, despite Strategy 2 combination variants having higher Tm values overall.

TABLE 7.3
Combinations of Strategy 3 Mutations

| | Chain A | Chain B | IIb- | IIaR- | IIb/IIaR | aSEC | aSEC | ΔTm/° |
|---|---|---|---|---|---|---|---|---|
| Variant # | Mutations¹ | Mutations¹ | Fold² | Fold² | Selectivity³ | Monomer/% | Aggregates/% | C.⁴ |
| 29724 | A237D_strat1 | template7_ | 44.5 | 0.7 | 62.1 | 91.3 | 3.2 | −7 |
| | | E328*H_E329*R_ | | | | | | |
| | | A331*BY_ | | | | | | |
| | | strat3 | | | | | | |
| 29725 | L235F_strat1 | template7_ | 153 | 4 | 38.1 | 87.9 | 3.2 | −3 |
| | | E328*H_E329*R_ | | | | | | |
| | | A331*BY_ | | | | | | |
| | | strat3 | | | | | | |
| 29726 | S239Y_strat1 | template7_ | 80.7 | 2.2 | 36 | 89.1 | 2.6 | −6 |
| | | E328*H_E329*R_ | | | | | | |
| | | A331*BY_ | | | | | | |
| | | strat3 | | | | | | |

¹⁻⁴See footnotes to Table 7.1. “Strat3” indicates the parental CH2 mutations are those of Launching Module 3.

As shown in Table 7.3, medium to high FcγRIIb selectivity was observed for Strategy 3 combination variants as compared to Strategy 1 and Strategy 2 combination variants. Overall, Strategy 3 combination variants demonstrated higher stability by aSEC and DSF.

TABLE 7.4
Combinations of Mutations from Strategies 1, 2 and 3

| | Chain A | Chain B | IIb- | IIaR- | IIb/IIaR | aSEC | aSEC | ΔTm/° |
|---|---|---|---|---|---|---|---|---|
| Variant # | Mutations¹ | Mutations¹ | Fold² | Fold² | Selectivity³ | Monomer/% | Aggregates/% | C.⁴ |
| 29727 | L235D_strat2 | D329*I_strat1 | 366 | 5.1 | 72.4 | 92.1 | 2.7 | −14 |
| 29728 | S267A_strat2 | D329*I_strat1 | 292 | 4.9 | 59.5 | 92.5 | 3.3 | −13 |
| 29729 | K330T_strat2 | D329*I_strat1 | 281 | 4.6 | 61.4 | 92.1 | 3.1 | −13 |
| 29730 | L235D_strat2 | G330*K_strat1 | 1448 | 29.9 | 48.5 | 90 | 2.9 | −12 |
| 29731 | S267A_strat2 | G330*K_strat1 | 1282 | 31.6 | 40.6 | 89.8 | 3.3 | −12 |
| 29732 | K330T_strat2 | G330*K_strat1 | 1333 | 29.9 | 44.6 | 91.6 | 3.2 | −12.5 |
| 29733 | L235D_strat2 | I332L_strat1 | 548 | 23.9 | 22.9 | 89.7 | 6.8 | −14.5 |
| 29734 | S267A_strat2 | I332L_strat1 | 694 | 29.7 | 23.4 | 95.6 | 2.7 | −14.5 |
| 29735 | K330T_strat2 | I332L_strat1 | 557 | 28 | 19.9 | 95.2 | 2.9 | −14 |

¹⁻⁴See footnotes to Table 7.1

Table 7.4 shows that combining mutations in Chain A from Strategy 2 with mutations in Chain B from Strategy 1 is beneficial. A preliminary hypothesis for this observation is that the IgG4 FcγRIIb selectivity comes in large part from Chain A.

### Example 8: Testing LVG2 in Full-Size Antibody Format

Combination variants from Example 7 showing the highest selectivity for FcγRIIb were selected and additional engineering conducted as described below to optimize these variants for transfer into full-size antibody (FSA) format. The selected variants are shown in Table 8.1.

TABLE 8.1
Variants Selected

| | Chain A | Chain B | IIb- | IIaR- | IIb/IIaR | aSEC | aSEC | ΔTm/° |
|---|---|---|---|---|---|---|---|---|
| Variant # | Mutations¹ | Mutations¹ | Fold² | Fold² | Selectivity³ | Monomer/% | Aggregates/% | C.⁴ |
| 29689 | L235F_strat1 | D329*I_strat1 | 550 | 6.3 | 87.5 | 92.4 | 2.7 | −10.5 |
| 29688 | A237D_strat1 | D329*I_strat1 | 98.4 | 1.2 | 79.2 | 93.7 | 2.4 | −14.5 |
| 29695 | L235F_strat1 | G330*K_strat1 | 1533 | 33.3 | 46 | 90.3 | 3 | −9.5 |
| 29715 | P329I_strat2 | G237D_strat2 | 115 | 3 | 38.7 | 89.8 | 3.3 | −12 |
| 29716 | L235D_strat2 | G237L_strat2 | 179 | 7.2 | 24.9 | 90.2 | 5.2 | −9.5 |
| 29724 | A237D_strat1 | template7_ | 44.5 | 0.7 | 62.1 | 91.3 | 3.2 | −7 |
| | | E328*H_E329*R_ | | | | | | |
| | | A331*BY_strat3 | | | | | | |
| 29727 | L235D_strat2 | D329*I_strat1 | 366 | 5.1 | 72.4 | 92.1 | 2.7 | −14 |

¹Mutation notation is in the format “A237D_strat1,” where “A237D” indicates the mutation made with “A” representing the parental residue being replaced, “237” representing the position and “D” representing the replacement residue, and “strat1” specifies the parental CH2 mutations (i.e. those of Launching Module 1)
²Fold change in affinity over wild-type
³Selectivity is defined as IIb-Fold/IIaR-Fold
⁴Compared to wild-type

The following considerations were addressed in the additional engineering round.

### 1. Potential Differences in Properties Between OAA and FSA Formats

Positions 236 and 237 are mutated in all the selected variants. To address the possibility that mutations at these positions in FSA format may impact the flexibility of the hinge region, glycine was re-introduced at position 237.

### 2. Confirming the Role of B_S267V

To confirm the role of the mutation S267V in Chain B as a binding enhancer, this mutation was reversed (i.e. mutated from valine (V) back to serine (S)). This reversal was expected to reduce FcγRIIb affinity by approximately 10-fold.

### 3. Testing Other Aromatics at Position 328

Changing the mutation at position 328* in the loop replacement from phenylalanine (F) to tyrosine (Y) was expected to be tolerated.

### 4. Stability

Most of the selected variants showed a decrease in Tm. To address this, the following three stability modules (from Example 5) were combined with the selected variants:
A287F_M428F
A287F_T250V
M428F_T250V

### 5. Selectivity

To try to improve FcγRIIb selectivity, some additional combinations of mutations were tested.

Variants were constructed in the following full-size antibody (FSA) scaffolds: trastuzumab (anti-HER2; Scaffold 3), anti-CD19 (Scaffold 4) and anti-CD40 (Scaffold 5). The final variants tested in FSA format are shown in Table 8.2.

TABLE 8.2
Variants Tested in FSA Format

| | | Yield (mg/L)¹ | | |
|---|---|---|---|---|
| Variant | | Anti- | Anti- | Anti- |
| # | Description | HER2 | CD19 | CD40 |
| 31186 | v29688_FSA | 71 | 140 | 56 |
| 31187 | v29689_FSA | 87 | 82 | 51 |
| 31188 | v29695_FSA | 61 | 21 | 30 |
| 31256 | v29715_FSA | 49 | 407 | 22 |
| 31190 | v29716_FSA | 79 | 152 | 26 |
| 31191 | v29724_FSA | 100 | 286 | 203 |
| 31192 | v29727_FSA | 86 | 180 | 23 |
| 31274 | v29689_FSA_Stability, add A287F_T250V | 105 | 180 | 21 |
| 31275 | v29689_FSA_Stability, add M428F_T250V | 70 | 264 | 41 |
| 31276 | v29689_FSA_Stability, add A287F_M428F | 54 | 168 | 24 |
| 31209 | v29689_FSA_B_F328*Y | 27 | 144 | 17 |
| 31210 | v29689_FSA_Remove binding enhancer B_V267S | 89 | 192 | 41 |
| 31211 | v29689_FSA_Test distant combinations A_A237D | 60 | 223 | 26 |
| 31212 | v29689_FSA_Test distant combinations B_P271D | 48 | 218 | 22 |
| 31213 | v29689_FSA_Test distant combinations B_I332L | 96 | 78 | 43 |
| 31214 | v29689_FSA_Test combination B_D329*I + G330*K | 74 | 32 | 80 |
| 31215 | v29689_FSA_B_D236K/F237G | 119 | 189 | 43 |
| 31216 | v29689_FSA_A_N236F/A237G | 95 | 64 | 10 |
| 31217 | v29689_FSA_A_N236F/A237G_B_D236K/F237G | 93 | 213 | 43 |
| 31253 | v29715_FSA_Stability, add A287F_M428F | 72 | 268 | 51 |
| 31278 | v29715_FSA_A_N236G | 71 | 339 | 19 |
| 31255 | v29724_FSA_Stability, add A287F_M428F | 47 | 446 | 41 |

¹Variants in the different scaffolds (anti-HER2, anti-CD19, anti-CD40) were purified by slightly different protocols. The results shown, therefore, provide a comparison of yield between variants within the same scaffold only.

FSA variants were tested for FcγR binding by SPR as described in the General Methods (Protocol 2). Thermal stability and aggregation propensity of the variants were measured by DSF and aSEC, respectively, as described in the General Methods.

### Results

FSAs have the Same Properties as their OAA Counterparts

As shown in Table 8.3, the seven variants tested in trastuzumab FSA and OAA formats showed very similar levels of binding affinity and selectivity across the different Fcγ receptors.

TABLE 8.3
Comparison of FcγRIIb Binding for Variants in OAA and FSA Formats

| | | | | | | | | | | Ratio |
|---|---|---|---|---|---|---|---|---|---|---|
| | Chain A | Chain B | FcγR Binding (Fold Change over WT) | | | | | | IIb/IIaR | FSA/OAA |
| Variant # | Mutations¹ | Mutations¹ | Ia | IIaH | IIaR | IIb | IIIaF | IIIaV | Selectivity² | Selectivity |

Strategy 1

| | | | | | | | | | | |
|---|---|---|---|---|---|---|---|---|---|---|
| 29689 | L235F_strat1 | D329*I_strat1 | NB³ | 0.1 | 6.3 | 549.9 | 0.1 | NB | 87.5 | 0.7 |
| (OAA) | | | | | | | | | | |
| 31187 | | | NB | 0.0 | 7.5 | 451.0 | NB | NB | 59.8 | |
| (FSA) | | | | | | | | | | |
| 29688 | A237D_strat1 | D329*I_strat1 | 0.0 | NB | 1.2 | 98.4 | 0.1 | NB | 79.2 | 1.0 |
| (OAA) | | | | | | | | | | |
| 31186 | | | 0.0 | NB | 1.4 | 95.3 | NB | NB | 69.2 | |
| (FSA) | | | | | | | | | | |
| 29695 | L235F_strat1 | G330*K_strat1 | 0.0 | 0.0 | 33.3 | 1533.3 | 0.1 | NB | 46.0 | 0.7 |
| (OAA) | | | | | | | | | | |
| 31188 | | | NB | 0.0 | 41.7 | 1300.8 | NB | NB | 31.2 | |
| (FSA) | | | | | | | | | | |

Strategy 2

| | | | | | | | | | | |
|---|---|---|---|---|---|---|---|---|---|---|
| 29715 | P329I_strat2 | G237D_strat2 | 0.0 | 0.0 | 3.0 | 115 | NB | NB | 38.7 | 0.8 |
| (OAA) | | | | | | | | | | |
| 31256 | | | 0.0 | 0.0 | 4.0 | 126 | NB | NB | 31.5 | |
| (FSA) | | | | | | | | | | |
| 29716 | L235D_strat2 | G237L_strat2 | 0.0 | 0.2 | 7.2 | 179 | 0.4 | 0.1 | 24.9 | 0.7 |
| (OAA) | | | | | | | | | | |
| 31190 | | | 0.0 | 0.1 | 10.6 | 185 | 0.3 | 0.0 | 17.4 | |
| (FSA) | | | | | | | | | | |

Strategy 3

| | | | | | | | | | | |
|---|---|---|---|---|---|---|---|---|---|---|
| 29724 | A237D_strat1 | template7_ | NB | NB | 0.7 | 44.5 | NB | NB | 62.1 | 1.0 |
| (OAA) | | E328*H_ | | | | | | | | |
| 31191 | | E329*R_ | NB | NB | 0.9 | 34.2 | NB | NB | 62.1 | |
| (FSA) | | A331*BY_ | | | | | | | | |
| | | strat3 | | | | | | | | |

Combination

| | | | | | | | | | | |
|---|---|---|---|---|---|---|---|---|---|---|
| 29727 | L235D_strat2 | D329*I_strat1 | 0.0 | 0.0 | 5.1 | 366 | NB | NB | 72.4 | 0.6 |
| (OAA) | | | | | | | | | | |
| 31192 | | | 0.0 | 0.0 | 5.3 | 243 | NB | NB | 45.7 | |
| (FSA) | | | | | | | | | | |

IIb-Specific Comparator

| | | | | | | | | | | |
|---|---|---|---|---|---|---|---|---|---|---|
| v12⁴ | E233D_ | E233D_ | 0 | 0.1 | 1.4 | 112 | 0.1 | NB | 82.9 | 0.9 |
| (OAA) | G237D_ | G237D_ | | | | | | | | |
| v12⁴ | P238D_ | P238D_ | 0.0 | 0.1 | 1.6 | 125 | NB | NB | 76.9 | |
| (FSA) | H268D_ | H268D_ | | | | | | | | |
| | P271G_ | P271G_ | | | | | | | | |
| | A330R | A330R | | | | | | | | |

¹Mutation notation is in the format “A237D_strat1,” where “A237D” indicates the mutation made with “A” representing the parental residue being replaced, “237” representing the position and “D” representing the replacement residue, and “strat1” specifies the parental CH2 mutations
²Selectivity is defined as IIb-Fold/IIaR-Fold
³NB = no binding
⁴Mimoto, et al., 2013, Protein Eng. Des. Sel., 26: 589-598

### Re-Introduction of G237 Reduced Selectivity

As shown in Table 8.4, re-introduction of glycine (G) at position 237 in Chain A of the Fe reduced FcγRIIb selectivity by approximately 30. Table 8.4 also shows that introduction of lysine (K) at position 236 on Chain B abrogated FcγRIIb binding in a v29689 background.

TABLE 8.4
Effect of Reversion to G237 and G236K Mutation

| | | | | | | | | | Ratio |
|---|---|---|---|---|---|---|---|---|---|
| | | | | | | | | | Mutant/ |
| | | FcγR Binding (Fold over WT) | | | | | | IIb/IIaR | Control |
| Variant # | Description | Ia | IIaH | IIaR | IIb | IIIaF | IIIaV | Selectivity¹ | Selectivity |
| 31187 | v29689_A_L235F_strat1 | NB² | 0.03 | 7.54 | 451 | NB | NB | 59.80 | |
| | B_D329*I_strat1 (control) | | | | | | | | |
| 31215 | v31187_B_D236K/F237G | NB | NB | NB | NE | NB | NB | N/A | |
| 31216 | v31187_A_N236F/A237G | 0.01 | 0.04 | 8.80 | 335 | NB | 0.02 | 38.09 | 64% |
| 31217 | v31187_A_N236F/A237G | 0.00 | NB | NB | NB | NB | NB | N/A | |
| | B_D236K/F237G | | | | | | | | |
| 31256 | v29715_A_P329I_strat2 | 0.01 | 0.02 | 4.00 | 126 | NB | NB | 31.53 | |
| | B_G237D_strat2 (control) | | | | | | | | |
| 31278 | v31256_A_N236G | 0.07 | 0.05 | 6.42 | 147 | NB | NB | 22.97 | 73% |

¹Selectivity is defined as IIb-Fold/IIaR-Fold
²NB = no binding

### Removal of S267 Affects FcγRIIb Affinity and Selectivity

The mutation S267V had been identified as a binding enhancer (see Example 1). The results shown in Table 8.5 confirm that this mutation is important for both affinity and selectivity for FcγRIIb when present together with the loop replacement. Reversal of this mutation decreased FcγRIIb affinity and selectivity. It is possible that this mutation plays a role with the D329*I mutation.

TABLE 8.5
Effect of S267V Mutation

| | | | | | | | | | Ratio |
|---|---|---|---|---|---|---|---|---|---|
| | | | | | | | | | Mutant/ |
| | | FcγR Binding (Fold over WT) | | | | | | IIb/IIaR | Control |
| Variant # | Description | Ia | IIaH | IIaR | IIb | IIIaF | IIIaV | Selectivity¹ | Selectivity |
| 31187 | v29689_A_L235F_strat1 | NB² | 0.03 | 7.54 | 451 | NB | NB | 59.80 | |
| | B_D329*I_strat1 (control) | | | | | | | | |
| 31210 | v31187_Remove binding | 0.00 | 0.07 | 2.99 | 61.6 | 0.11 | 0.03 | 20.59 | 34% |
| | enhancer B_V267S | | | | | | | | |

¹Selectivity is defined as IIb-Fold/IIaR-Fold
²NB = no binding

### Mutation 328*Y Maintains FcγRIIb Affinity and Selectivity

The results shown in Table 8.6 show that changing the mutation at position 328* from phenylalanine (F) to tyrosine (Y) in a v29689 background does not impact FcγRIIb affinity or selectivity.

TABLE 8.6
Effect of 328*Y Mutation

| | | | | | | | | | Ratio |
|---|---|---|---|---|---|---|---|---|---|
| | | | | | | | | | Mutant/ |
| | | FcγR Binding (Fold over WT) | | | | | | IIb/IIaR | Control |
| Variant # | Description | Ia | IIaH | IIaR | IIb | IIIaF | IIIaV | Selectivity¹ | Selectivity |
| 31187 | v29689_A_L235F_strat1 | NB² | 0.03 | 7.54 | 451 | NB | NB | 59.8 | |
| | B_D329*I_strat1 (control) | | | | | | | | |
| 31209 | v31187_B_F328*Y | 0 | 0.03 | 9.82 | 560 | NB | NB | 57.03 | 95% |

¹Selectivity is defined as IIb-Fold/IIaR-Fold
²NB = no binding

### Stability Modules Did not Affect FcγRIIb Affinity or Selectivity

The stability modules were tested in three different variants. As shown in Table 8.7, no significant change in FcγRIIb affinity or selectivity was observed by inclusion of the stability modules in any of the tested variants.

TABLE 8.7
Effect of Stability Mutations

| | | | | | | | | | Ratio |
|---|---|---|---|---|---|---|---|---|---|
| | | | | | | | | | Mutant/ |
| | | FcγR Binding (Fold Change over WT) | | | | | | IIb/IIaR | Control |
| Variant # | Description | Ia | IIaH | IIaR | IIb | IIIaF | IIIaV | Selectivity¹ | Selectivity |
| 31187 | v29689_A_L235F_strat1 | NB² | 0.03 | 7.54 | 451 | NB | NB | 59.80 | |
| | B_D329*I_strat1 (control) | | | | | | | | |
| 31274 | v31187_Stability, add | 0 | 0.04 | 8.19 | 439 | NB | NB | 53.61 | 90% |
| | A287F_T250V on both chains | | | | | | | | |
| 31275 | v31187_Stability, add | NB | 0.04 | 8.82 | 539 | NB | NB | 61.07 | 102% |
| | M428F_T250V on both chains | | | | | | | | |
| 31276 | v31187_Stability, add | NB | 0.03 | 8.44 | 479 | NB | NB | 56.79 | 95% |
| | A287F_M428F on both chains | | | | | | | | |
| 31256 | v29715_A329I_strat2 | 0.01 | 0.02 | 4.00 | 126 | NB | NB | 31.53 | |
| | B_G237D_strat2 (control) | | | | | | | | |
| 31253 | v31256_Stability, add | 0.01 | 0.02 | 4.15 | 138 | NB | NB | 33.23 | 105% |
| | A287F_M428F on both chains | | | | | | | | |
| 31191 | v29724_A237D_strat1 | NB | NB | 0.89 | 34.2 | NB | NB | 38.34 | |
| | B_template7_E328*H | | | | | | | | |
| | E329*R_A331*BY_strat3 | | | | | | | | |
| | (control) | | | | | | | | |
| 31255 | v31191_Stability, add | NB | NB | 0.95 | 43 | NB | NB | 45.16 | 118% |
| | A287F_M428F on both chains | | | | | | | | |

¹Selectivity is defined as IIb-Fold/IIaR-Fold
²NB = no binding

### New Combinations of Mutations Showed Similar FcγRIIb Affinity and Selectivity

As shown in Table 8.8, the new combinations of mutations tested showed FcγRIIb affinity and/or selectivity that was equivalent to or lower than variant v01187.

TABLE 8.8
Additional Combinations

| | | | | | | | | | Ratio |
|---|---|---|---|---|---|---|---|---|---|
| | | | | | | | | | Mutant/ |
| | | FcγR Binding (Fold Change over WT) | | | | | | IIb/IIaR | Control |
| Variant # | Description | Ia | IIaH | IIaR | IIb | IIIaF | IIIaV | Selectivity¹ | Selectivity |
| 31187 | v29689_A_L235F_strat1 | NB² | 0.03 | 7.54 | 451 | NB | NB | 59.80 | |
| | B_D329*I_strat1 (control) | | | | | | | | |
| 31210 | v31187_Remove binding | 0 | 0.07 | 2.99 | 61.6 | 0.11 | 0.03 | 20.59 | 34% |
| | enhancer B_V267S | | | | | | | | |
| 31211 | v31187_Test distant | 0 | NB | 1.66 | 85.7 | NB | NB | 51.47 | 86% |
| | combinations A_A237D | | | | | | | | |
| 31212 | v31187_Test distant | 0 | 0.03 | 2.99 | 162 | NB | NB | 54.27 | 91% |
| | combinations B_P271D | | | | | | | | |
| 31213 | v31187_Test distant | 0 | 0.03 | 7.1 | 492 | NB | NB | 69.26 | 116% |
| | combinations B_I332L | | | | | | | | |
| 31214 | v31187_Test combination | NB | 0.03 | 8.78 | 295 | NB | NB | 33.62 | 56% |
| | D329*I + E330*K | | | | | | | | |

¹Selectivity is defined as IIb-Fold/IIaR-Fold
²NB = no binding

### Stability of FSAs

As shown in Table 8.9, the thermal stability of the tested variants in the three different systems (trastuzumab, anti-CD19 and anti-CD40) was similar, with the exception of variants v31215 and v31217. These variants showed a good stability in the trastuzumab and anti-CD40 background, but lower stability in the anti-CD19 background. Variants v31215 and v1217 include the mutation 236K, which lowers FcγRIIb affinity and selectivity.

TABLE 8.9
Thermal Stability of FSA Variants

| | | Tm/° C. | | | |
|---|---|---|---|---|---|
| | | DSC | DSF | | |
| Variant | | Anti- | Anti- | Anti- | Anti- |
| # | Description | HER2 | HER2 | CD19 | CD40 |
| 21653 | FSA_WT | 71.7 | 68.5 | | |
| v12¹ | FSA_IIb-Specific Comparator | 64.0 | 61.5 | | |

Strategy 1

| | | | | | |
|---|---|---|---|---|---|
| 31187 | v29689_A_L235F_strat1 B_D329*I_strat1 | 61.8 | 59.0 | 59.0 | 59.0 |
| 31186 | v29688_A_A237D_strat1 B_D329*I_strat1 | 58.9 | 55.5 | 55.0 | 55.5 |
| 31188 | v29695_A_L235F_strat1 B_G330*K_strat1 | 62.5 | 60.0 | 59.5 | 60.0 |

Strategy 2

| | | | | | |
|---|---|---|---|---|---|
| 31256 | v29715_A_P329I_strat2 B_G237D_strat2 | 60.2 | 58.0 | 57.0 | 57.5 |
| 31190 | v29716_A_L235D_strat2 B_G237L_strat2 | 63.8 | 60.0 | 60.0 | 60.5 |

Strategy 3

| | | | | | |
|---|---|---|---|---|---|
| 31191 | v29724_A_A237D_strat1 | 65.0 | 62.5 | 63.0 | 62.5 |
| | B_template7_E328*H_E329*R _A331*BY_strat3 | | | | |

Combinations

| | | | | | |
|---|---|---|---|---|---|
| 31192 | v29727_A_L235D_strat2 B_D329*I_strat1 | 59.6 | 57.0 | 56.5 | 56.5 |

Strategy 1 Variations

| | | | | | |
|---|---|---|---|---|---|
| 31209 | v31187_B_F328*Y | 62.1 | 59.5 | 59.0 | 59.5 |
| 31210 | v31187_Remove binding enhancer B_V267S | 62.0 | 59.5 | 59.0 | 59.0 |
| 31211 | v31187_Test distant combinations A_A237D | 58.3 | 55.5 | 55.0 | 55.0 |
| 31212 | v31187_Test distant combinations B_P271D | 62.3 | 59.5 | 59.5 | 59.5 |
| 31213 | v31187_Test distant combinations B_1332L | 60.4 | 58.5 | 58.0 | 58.5 |
| 31214 | v31187_Test combination D329*I + E330*K | 60.1 | 59.5 | 59.5 | 59.5 |
| 31215 | v31187_B_D236K/F237G | 67.6 | 64.5 | 54.0 | 65.0 |
| 31216 | v31187_A_N236F/A237G | 61.2 | 58.5 | 58.5 | 59.0 |
| 31217 | v31187_A_N236F/A237G B_D236K/F237G | 67.5 | 64.0 | 53.5 | 64.5 |
| 31274 | v31187_Stability, add A287F_T250V on both | 71.6 | 68.0 | 67.5 | 68.5 |
| | chains | | | | |
| 31275 | v31187_Stability, add M428F_T250V on both | 70.2 | 67.0 | 67.0 | 68.0 |
| | chains | | | | |
| 31276 | v31187_Stability, add A287F_M428F on both | 68.0 | 65.0 | 67.0 | 65.0 |
| | chains | | | | |

Strategy 2 Variations

| | | | | | |
|---|---|---|---|---|---|
| 31253 | v31256_Stability, add A287F_M428F on both | 67.4 | 64.0 | 64.0 | 64.0 |
| | chains | | | | |
| 31278 | v31256_A_N236G | 57.4 | 57.5 | 57.0 | 57.5 |

Strategy 3 Variations

| | | | | | |
|---|---|---|---|---|---|
| 31255 | v31191_Stability, add A287F_M428F on both | 71.2 | 68.5 | 68.0 | 68.5 |
| | chains | | | | |

¹Symmetrical E233D_G237D_P238D_H268D_P271G_A330R mutations (Mimoto, et al., 2013, Protein Eng. Des. Sel., 26: 589-598)

The results shown in Table 8.9 also indicate that inclusion of the stability modules in the three selected variants increased the thermal stability of the variants such that the CH2 Tm was close to that of wild-type. As shown in Table 8.10 below, the effect was observed across all three tested variants and all three FSA systems providing a strong indication that the stability modules are transferable.

TABLE 8.10
Stability Modules increase the Tm of Test FSA Variants

| | | Tm/° C. | | | | Anti- |
|---|---|---|---|---|---|---|
| | | DSC | DSF | | | HER2 |
| | | Anti- | Anti- | Anti- | Anti- | Mutant/ |
| Variant | Description | HER2 | HER2 | CD19 | CD40 | WT Δ Tm |
| 21653 | FSA WT | 71.7 | 68.5 | | | 0 |

Strategy 1

| | | | | | | |
|---|---|---|---|---|---|---|
| 31187 | v29689_A_L235F_strat1 | 61.8 | 59 | 59 | 59 | −9.9 |
| | B_D329*I_strat1 | | | | | |
| 31274 | v31187_Stability, add A287F_T250V on | 71.6 | 68 | 67.5 | 68.5 | −0.2 |
| | both chains | | | | | |
| 31275 | v31187_Stability, add M428F_T250V on | 70.2 | 67 | 67 | 68 | −1.5 |
| | both chains | | | | | |
| 31276 | v31187_Stability, add A287F_M428F on | 68 | 65 | 67 | 65 | −3.8 |
| | both chains | | | | | |

Strategy 2

| | | | | | | |
|---|---|---|---|---|---|---|
| 31256 | v29715_A_P329I_strat2 B_G237D_strat2 | 60.2 | 58 | 57 | 57.5 | −11.5 |
| 31253 | v31256_Stability, add A287F_M428F on | 67.4 | 64 | 64 | 64 | −4.3 |
| | both chains | | | | | |

Strategy 3

| | | | | | | |
|---|---|---|---|---|---|---|
| 31191 | v29724_A_A237D_strat1 | 65 | 62.5 | 63 | 62.5 | −6.8 |
| | B_template7_E328*H_E329*R_A331*BY_{—} | | | | | |
| | strat3 | | | | | |
| 31255 | v31191_Stability, add A287F_M428F on | 71.2 | 68.5 | 68 | 68.5 | −0.5 |
| | both chains | | | | | |

Analytical SEC of the tested variants showed that all variants contained >85% monomeric species. All variants in the anti-HER2 scaffold contained >95% monomeric species and thus had a very low tendency to aggregate (see Table 8.11).

TABLE 8.11
aSEC Analysis of_FSA_Variants

| | | aSEC % Monomer | | |
|---|---|---|---|---|
| Variant | | Anti- | Anti- | Anti- |
| # | Description | HER2 | CD19 | CD40 |
| 21653 | FSA_WT | 97.5 | | |
| v12¹ | FSA_IIb-Specific Comparator | 92.8 | | |

Strategy 1

| | | | | |
|---|---|---|---|---|
| 31187 | v29689_A_L235F_strat1 B_D329*I_strat1 | 100.0 | 89.5 | 93.1 |
| 31186 | v29688_A_A237D_strat1 B_D329*I_strat1 | 98.5 | 96.7 | 95.8 |
| 31188 | v29695_A_L235F_strat1 B_G330*K_strat1 | 99.4 | 93.5 | 91.5 |

Strategy 2

| | | | | |
|---|---|---|---|---|
| 31256 | v29715_A_P329I_strat2 B_G237D_strat2 | 99.3 | 88.5 | 87.1 |
| 31190 | v29716_A_L235D_strat2 B_G237L_strat2 | 98.8 | 94.8 | 94.6 |

Strategy 3

| | | | | |
|---|---|---|---|---|
| 31191 | v29724_A_A237D_strat1 | 96.9 | 92.9 | 70.2 |
| | B_template7_E328*H_E329*R_A331*BY_strat3 | | | |

Combinations

| | | | | |
|---|---|---|---|---|
| 31192 | v29727_A_L235D_strat2 B_D329*I_strat1 | 99.0 | 87.0 | 96.0 |

Strategy 1 Variations

| | | | | |
|---|---|---|---|---|
| 31209 | v31187_B_F328Y | 99.7 | 91.2 | 90.8 |
| 31210 | v31187_Remove binding enhancer B_V267S | 99.8 | 90.3 | 89.0 |
| 31211 | v31187_Test distant combinations A_A237D | 99.7 | 93.3 | 93.3 |
| 31212 | v31187_Test distant combinations B_P271D | 99.5 | 86.9 | 90.9 |
| 31213 | v31187_Test distant combinations B_I332L | 99.4 | 90.0 | 91.8 |
| 31214 | v31187_Test combination D329*I + E330*K | 98.0 | 90.1 | 85.5 |
| 31215 | v31187_B_D236K/F237G | 99.4 | 83.4 | 90.3 |
| 31216 | v31187_A_N236F/A237G | 99.4 | 89.1 | 95.6 |
| 31217 | v31187_A_N236F/A237G B_D236K/F237G | 99.5 | 80.7 | 95.2 |
| 31274 | v31187_Stability, add A287F_T250V on both chains | 99.5 | 95.1 | 94.2 |
| 31275 | v31187_Stability, add M428F_T250V on both chains | 99.3 | 90.7 | 94.3 |
| 31276 | v31187_Stability, add A287F_M428F on both chains | 100.0 | 92.3 | 95.5 |

Strategy 2 Variations

| | | | | |
|---|---|---|---|---|
| 31253 | v31256_Stability, add A287F_M428F on both chains | 98.2 | 82.4 | 94.5 |
| 31278 | v31256_A_N236G | 99.8 | 92.7 | 91.6 |

Strategy 3 Variations

| | | | | |
|---|---|---|---|---|
| 31255 | v31191_Stability, add A287F_M428F on both chains | 98.6 | 91.3 | 94.6 |

¹Symmetrical E233D_G237D_P238D_H268D_P271G_A330R mutations (Mimoto, et al., 2013, Protein Eng. Des. Sel., 26: 589-598)

### Example 9: Asymmetric G236 Mutations

In Example 1, G236 was identified as a promising position in the IgG lower hinge region for introducing mutations to drive FcγRIIb selectivity. This position is close to positions 135 and 163 in the Fcγ receptor in the Fc-FcγR complex and hence can drive selectivity.

The mutations G236N and G236D were each shown to modestly improve FcγRIIb selectivity in Example 1. Interestingly, G236N and G236D appeared to have opposite polarities, with G236N being identified as a Chain A mutation and G236D being identified as a Chain B mutation, which suggested that these two mutations could be combined on opposite chains to improve selectivity. Additional variants as described below were generated and tested in order to investigate further the effect of asymmetric mutations at this position.

An initial round of variants was generated that included the G236N and/or G236D mutations in combination with mutations that had been identified as FcγRIIb binding enhancer mutations in Example 1 in order to increase FcγRIIb affinity.

This initial round of variants also included variants designed to address a potential deamidation liability. Specifically, the mutations G236N and G236D are followed by glycine at position 237 and thus both mutations could potentially introduce a deamidation site. To address this potential liability, substitutions at these positions with glutamine (Q), histidine (H) or glutamate (E) were also tested. In addition, the combinations G236N_G237A and G236N_G237F were tested.

Additional G236 asymmetric mutations were identified by in silico packing. All possible 400 amino acid combinations for chain A and chain B G236 mutations were packed and analyzed based on AMBER affinity and DDRW affinity.

The top mutations that created the largest differences in AMBER affinity were selected and filtered using the following criteria:
1. van der Waals (VDW) overlap for FcγRIIbY<0.3A (packs with significant clashes removed)
2. AMBER affinity for FcγRIIbY<5 kcal mol⁻¹
3. AMBER affinity for FcγRIIbY—AMBER affinity for FcγRIIaR<−10 (selectivity metric)
4. AMBER affinity for FcγRIIbY—AMBER affinity for FcγRIIaH<−4 (selectivity towards FcγRIIaH also considered).

The top mutations that created the largest differences in DDRW affinity were selected and filtered using the following criteria:
1. VDW overlap for FcγRIIbY<0.25A (packs with significant clashes removed)
2. DDRW affinity for FcγRIIbY—DDRW affinity for FcγRIIaR<−50 (selectivity metric)
3. DDRW affinity for FcγRIIbY—DDRW affinity for FcγRIIaH<0 (selectivity towards FcγRIIaH also considered).

The in silico packing analysis identified the following 4 additional mutations for testing:
A_G236N B_G236S
A_G236L B_G236E
A_G236D B_G236E
A_G236D B_G236H.

Variants were constructed in a one-armed antibody scaffold (Scaffold 2) and tested for FcγR binding by SPR as described in the General Methods (Protocol 2). The results are shown in Table 9.1.

TABLE 9.1
Effect of G236 Mutations on FcγR Binding

| | | | | | | |
|---|---|---|---|---|---|---|
| | Chain A | Chain B | IIb- | IIaR- | IIb/IIaR | |
| Variant # | Mutations | Mutations | Fold¹ | Fold¹ | Selectivity² | Comments |

Symmetric Mutations

| | | | | | | |
|---|---|---|---|---|---|---|
| 16490 | G236D | G236D | 2.1 | 0.9 | 2.4 | Increases selectivity |
| 19699 | G236D_G237F | G236D_G237F | 2.1 | 0.3 | 7 | Increases selectivity and |
| | | | | | | removes potential deamidation |
| | | | | | | site |
| 16493 | G236N | G236N | 0.8 | 0.3 | 3.1 | Increases selectivity |
| 19503 | G236N_G237A | G236N_G237A | 0.2 | 0.04 | 4.5 | Removes potential deamidation |
| | | | | | | site, but binding decreases |
| 19504 | G236N_G237F | G236N_G237F | 0.2 | 0.05 | 3.3 | Removes potential deamidation |
| | | | | | | site, but binding decreases |
| 19505 | G236H | G236H | 0.2 | 0.2 | 1 | Reduces affinity and no |
| | | | | | | improvement in selectivity |
| 19506 | G236Q | G236Q | 0.2 | 0.2 | 1 | Reduces affinity and no |
| | | | | | | improvement in selectivity |
| 19507 | G236E | G236E | 0.8 | 1.8 | 0.4 | Reduces affinity and increases |
| | | | | | | IIaR selectivity |

Asymmetric Mutations

| | | | | | | |
|---|---|---|---|---|---|---|
| 19508 | | S239D_H268D | 31.1 | 25.6 | 1.2 | Control |
| 19509 | G236N | S239D_H268D | 31.8 | 9.7 | 3.3 | Increases selectivity and |
| | | | | | | retains affinity |
| 19510 | G236N_G237A | S239D_H268D | 5.5 | 1.8 | 3.1 | Affinity deceases, but |
| | | | | | | selectivity retained |
| 19511 | G236N_G237F | S239D_H268D | 7.1 | 5.7 | 1.2 | Loss of selectivity |
| 19512 | | G236N_S239D_{—} | 7.9 | 3.2 | 2.5 | Decreases affinity |
| | | H268D | | | | |
| 19513 | G236H | S239D_H268D | 8.2 | 13.4 | 0.6 | No selectivity |
| 19514 | | G236H_S239D_{—} | 4.2 | 2.5 | 1.7 | Loss of selectivity |
| | | H268D | | | | |
| 19515 | G236Q | S239D_H268D | 9.6 | 14.2 | 0.7 | No selectivity |
| 19516 | | G236Q_S239D_{—} | 7.4 | 7.6 | 1 | No selectivity |
| | | H268D | | | | |
| 19517 | G236D | S239D_H268D | 30.5 | 11.6 | 2.6 | Increases selectivity |
| 19518 | | G236D_S239D_{—} | 32.9 | 14.7 | 2.2 | Increases selectivity |
| | | H268D | | | | |
| 19694 | | G236D_G237F_{—} | 53.5 | 18.2 | 2.9 | Increases selectivity and |
| | | S239D_H268D | | | | affinity, removes potential |
| | | | | | | deamidation site |
| 19519 | G236E | S239D_H268D | 10.3 | 30.9 | 0.3 | Increases IIaR selectivity |
| 19520 | | G236E_S239D_{—} | 26.5 | 17.5 | 1.5 | No selectivity |
| | | H268D | | | | |
| 19521 | G236N | G236D_S239D_{—} | 37.6 | 5.5 | 6.8 | Increases selectivity |
| | | H268D | | | | and affinity |
| 19522 | G236N | Template(1) + | 69.6 | 11.7 | 5.9 | Increases selectivity |
| | | G236D_S239D_{—} | | | | and affinity |
| | | H268D | | | | |
| 19523 | G236D | G236N_S239D_{—} | 6.9 | 1.9 | 3.6 | Increases selectivity, but |
| | | H268D | | | | decreases affinity - not as |
| | | | | | | effective as opposite design |
| 19524 | G236Q | G236D_S239D_{—} | 10.3 | 8.9 | 1.2 | No selectivity |
| | | H268D | | | | |
| 19525 | G236D | G236Q_S239D_{—} | 7.7 | 2.1 | 3.7 | Increases selectivity, but |
| | | H268D | | | | decreases affinity - no |
| | | | | | | improvement over G236D in |
| | | | | | | chain A alone (v19517) |
| 19526 | G236D | G236K_S239D_{—} | 1.4 | 0.4 | 3.5 | Increases selectivity, but |
| | | H268D | | | | decreases affinity - no |
| | | | | | | improvement over G236D in |
| | | | | | | chain A alone (v19517) |
| 19527 | G236N | G236K_S239D_{—} | 1.2 | 0.2 | 6 | Increases selectivity, but |
| | | H268D | | | | decreases affinity - no |
| | | | | | | improvement in selectivity over |
| | | | | | | G236D in chain B (v19521) |
| 19528 | G236N | G236S_S239D_{—} | 18.6 | 6.7 | 2.8 | Low selectivity |
| | | H268D | | | | |
| 19589 | G236L | G236E_S239D_{—} | 2.5 | 2.4 | 1 | No selectivity |
| | | H268D | | | | |
| 19530 | G236D | G236E_S239D_{—} | 27.7 | 9.3 | 3 | Low selectivity |
| | | H268D | | | | |
| 19531 | G236D | G236H_S239D_{—} | 3.7 | 1.6 | 2.3 | Low selectivity |
| | | H268D | | | | |

¹Fold change in affinity over wild-type
²Selectivity is defined as IIb-Fold/IIaR-Fold

The results in Table 9.1 show that G236 is a very important residue for affinity and selectivity of Fc binding to the FcγRIIb and FcγRIIaR receptors. As shown in Table 9.1, the effect of symmetric and asymmetric mutations in this position were tested in the context of the S239D/H268D binding enhancers, which increase non-selective binding to both FcγRIIb and FcγRIIaR receptors (see Table 9.1, v19508, selectivity=1.2). The mechanism for this enhancement is the introduction of negative charges that interact with positive charges common between the two receptors. For example, S239D can form an H-bond with K120 in the receptors, and H268D is proximal to K131 in the receptors. This binding enhancement is effective only when the S239D/H268D mutations are placed in the Fc chain that is equivalent to chain B of the 1E4K Fc/FcγRIIIb structure (see FIG. 9). The same asymmetric mutations in the opposite chain (chain A) do not have an equivalent positively charged partner. Hence, testing the effect of mutations at position G236 with these asymmetric binding enhancers provides insight into the asymmetric mechanism of the selectivity and/or affinity changes of the G236 mutations.

The mutations G236D and G236N when each introduced symmetrically into both chains of the Fc were found to have positive effects on selectivity for FcγRIIb (see Example 1). When the mutation G236N was placed asymmetrically in conjunction with S239D/H268D, the results showed that G236N is most effective in driving FcγRIIb selectivity when placed as a chain A mutation. This confirms the results obtained with the E269K polarity driver (shown in Table 1.9). Specifically, Table 9.1 shows that the G236N mutation had higher FcγRIIb selectivity when positioned in the opposite chain to the S239D/H268D mutations (v19509, FcγRIIb affinity fold increase=32, selectivity=3.3) rather than in the same chain (v19512, FcγRIIb affinity fold increase=7.9, selectivity=2.5). On the other hand, the mutation G236D had a similar effect on FcγRIIb binding whether it was placed on the same chain or the opposite chain to the binding enhancers (v19517 (same chain) FcγRIIb affinity fold increase=30.5, selectivity=2.6; v19518 (opposite chain), FcγRIIb affinity fold increase=32.9, selectivity=2.2).

Given the above, the best FcγRIIb selectivity achieved with these mutations was when G236N was placed on chain A, and G236D was placed on chain B together with the binding enhancers S239D/H268D (see v19521, FcγRIIb affinity fold increase=37.6, selectivity=6.8). The opposite orientation (v19523) was still effective, but showed lower FcγRIIb selectivity (3.6) and affinity (6.9). In addition, the asymmetric combination (A_G236N B_G236D) in conjunction with the non-selective binding enhancers S239D/H268D had higher selectivity (selectivity=6.8) than the symmetric G236N mutations (v16493, selectivity=5.0) and the symmetric G236D mutations (v16490, selectivity=2.7).

### Example 10: FcRn Binding

Variants constructed in the trastuzumab full-size antibody (FSA) scaffold (Scaffold 3) (see Table 8.2) were tested for FcRn binding as described in the General Methods. The results are shown in Table 10.1.

TABLE 10.1
FcRn Binding of_FSA_Format Variants

| | | FcRn |
|---|---|---|
| Variant | | Binding |
| # | Description | (K_{D}/M) |
| 21653 | Wild-Type | 3.14E−07 |
| 31186 | v29688_FSA | 3.52E−07 |
| 31187 | v29689_FSA | 4.24E−07 |
| 31188 | v29695_FSA | 5.74E−07 |
| 31256 | v29715_FSA | 4.55E−07 |
| 31190 | v29716_FSA | 6.1E−07 |
| 31191 | v29724_FSA | 2.86E−07 |
| 31192 | v29727_FSA | 5.8E−07 |
| 31274 | v29689_FSA_Stability, add A287F_T250V | 4.05E−07 |
| 31275 | v29689_FSA_Stability, add M428F_T250V | 4.28E−07 |
| 31276 | v29689_FSA_Stability, add A287F_M428F | 5.03E−07 |
| 31209 | v29689_FSA_F328*Y | 4.65E−07 |
| 31210 | v29689_FSA_Remove binding enhancer | 6.46E−07 |
| | B_V267S | |
| 31211 | v29689_FSA_Test distant combinations | 1.98E−07 |
| | A_A237D | |
| 31212 | v29689_FSA_Test distant combinations | 2.84E−07 |
| | B_P271D | |
| 31213 | v29689_FSA_Test distant combinations | 3.45E−07 |
| | B_I332L | |
| 31214 | v29689_FSA_Test combination D329*I + G330*K | 3.36E−07 |
| 31215 | v29689_FSA_B_D236K/F237G | 3.57E−07 |
| 31216 | v29689_FSA_A_N236F/A237G | 3.6E−07 |
| 31217 | v29689_FSA_A_N236F/A237G | 3.69E−07 |
| | B_D236K/F237G | |
| 31253 | v29715_FSA_Stability, add A287F_M428F | 2.91E−07 |
| 31278 | v29715_FSA_A_N236G | 2.98E−07 |
| 31255 | v29724_FSA_Stability, add A287F_M428F | 2.93E−07 |
| v12 | v12¹_FSA | 3.05E−07 |

¹Mimoto, et al., 2013, Protein Eng. Des. Sel., 26: 589-598

The results indicated that the mutations tested did not have a measurable effect on FcRn binding.

### Example 11: C1q Binding

Variants constructed in the trastuzumab full-size antibody (FSA) scaffold (Scaffold 3) (see Table 8.2) were tested for C1q binding as described in the General Methods. The results are shown in Table 11.1.

TABLE 11.1
C1q Binding of Variants in FSA Format

| | | | | | | C1q |
|---|---|---|---|---|---|---|
| | | | | | C1q | Binding |
| | | | IIb- | IIb | Binding | Potency |
| Strategy | Variant # | Description | Fold¹ | Selectivity² | (% WT)³ | (% WT)⁴ |
| Strat 1 | 31187 | v29689_FSA | 451.0 | 59.8 | 160 | 817 |
| | 31186 | v29688_FSA | 95.3 | 69.2 | 69 | 50 |
| | 31188 | v29695_FSA | 1300.8 | 31.2 | 160 | 896 |
| Strat 2 | 31256 | v29715_FSA | 126.3 | 31.5 | 8 | b.d.⁵ |
| | 31190 | v29716_FSA | 184.7 | 17.4 | 9 | b.d. |
| Strat 3 | 31191 | v29724_FSA | 34.2 | 38.3 | 24 | b.d. |
| Combo | 31192 | v29727_FSA | 242.6 | 45.7 | 8 | b.d. |
| Strat 1 | 31209 | v29689_FSA_F328*Y | 559.8 | 57.0 | 161 | 989 |
| | 31210 | v29689_FSA_Remove | 61.6 | 20.6 | 162 | 808 |
| | | binding enhancer B_V267S | | | | |
| | 31211 | v29689_FSA_Test distant | 85.7 | 51.5 | 105 | 109 |
| | | combinations A_A237D | | | | |
| | 31212 | v29689_FSA_Test distant | 162.3 | 54.3 | 159 | 810 |
| | | combinations B_P271D | | | | |
| | 31213 | v29689_FSA_Test distant | 491.6 | 69.3 | 157 | 622 |
| | | combinations B_I332L | | | | |
| | 31214 | v29689_FSA_Test combination | 295.2 | 33.6 | 159 | 824 |
| | | D329*I + G330*K | | | | |
| | 31215 | v29689_FSA_B_D236K/F237G | NB | N/A | 55 | 28 |
| | 31216 | v29689_FSA_A_N236F/A237G | 335.1 | 38.1 | 163 | 677 |
| | 31217 | v29689_FSA_A_N236F/A237G_{—} | NB | N/A | 34 | 11 |
| | | B_D236K/F237G | | | | |
| | 31274 | v29689_FSA_Stability, add | 439.1 | 53.6 | 162 | 857 |
| | | A287F_T250V | | | | |
| | 31275 | v29689_FSA_Stability, add | 538.6 | 61.1 | 161 | 739 |
| | | M428F_T250V | | | | |
| | 31276 | v29689_FSA_Stability, add | 479.3 | 56.8 | 160 | 888 |
| | | A287F_M428F | | | | |
| Strat 2 | 31256 | v29715_FSA | 126.3 | 31.5 | 8 | b.d. |
| | 31253 | v29715_FSA_Stability, add | 138.0 | 33.2 | 8 | b.d. |
| | | A287F_M428F | | | | |
| | 31278 | v29715_FSA_A_N236G | 147.3 | 23.0 | 11 | b.d. |
| Strat 3 | 31191 | 29724_FSA | 34.2 | 38.3 | 24 | b.d. |
| | 31255 | v29724_FSA_Stability, add | 43.0 | 45.2 | 18 | b.d. |
| | | A287F_M428F | | | | |

¹Fold change in affinity over wild-type (values from Example 8)
²Selectivity is defined as IIb-Fold/IIaR-Fold (values from Example 8)
³Binding signal at 2 μg/ml C1q expressed as % of the wild-type (WT) control
⁴Relative C1q binding potency calculated as the concentration of C1q required to exceed the threshold signal of 17% of assay maximum expressed as a % of the WT control
⁵b.d. = below detection.

As can be seen from Table 11.1, the FSA based on variant v29689 showed higher C1q binding than wild-type. Introducing the mutation A237D decreased C1q binding close to the level of wild-type, while maintaining FcγRIIb selectivity.

FSA based on variant v29688, which also includes the mutation A237D, similarly showed decreased binding to C1q. This variant also lacks the L235F mutation, which appears to contribute to C1q binding.

Variants based on Strategy 2, Strategy 3 and Combination Strategy mutations did not show C1q binding.

### Example 12: Transferability to Other Heterodimeric Scaffolds

### 1. Selection of Heterodimer Scaffolds and Selectivity Variants

The variants v29689, v29715 and v29724 (see Table 8.1) were selected to assess whether the FcγRIIb selectivity-enhancing mutations are transferable to other heterodimeric Fc scaffolds.

These variants were originally constructed in an Azymetric (Azym) heterodimeric Fc scaffold (see International Patent Application Publication No. WO 2013/063702). The following additional heterodimeric Fc scaffolds were selected as test scaffolds:
1. Knobs-into-holes (K/H) (see Merchant, et al., 1998, Nat Biotechnol., 16(7):677-681)
2. Electrostatic steering (E/S) (see Gunasekaran, et al., 2010, J Biol Chem, 285(25): 19637-19646).

The CH3 mutations comprised by each of these scaffolds are shown in Table 11.1.

For variant v29689, the selectivity mutations in the CH2 domain were also tested in two orientations with respect to the CH3 domain mutations to demonstrate that the position of the mutations in the CH2 domain relative to the position of the mutations in the CH3 domain does not affect FcγR selectivity.

The variants tested are summarized in Table 12.1.

TABLE 12.1
Variants Tested for Transferability to Other Heterodimer Fc Scaffolds

| | Variant | | Chain A Mutations | | Chain B Mutations | |
|---|---|---|---|---|---|---|
| Variant # | Origin | Heterodimer | CH2 | CH3 | CH2 | CH3 |
| 31509 | | Azym | | T350V_L351Y_{—} | | T350V_T366L_{—} |
| | | | | F405A_Y407V | | K392L_T394W |
| 31521 | | K/H | | Y349C_T366S_{—} | | S354C_T366W |
| | | | | L368A_Y407V | | |
| 31522 | | E/S | | K392D_K409D | | E356K_D399K |
| 31523 | 29689 | Azym | G236N_G237A_{—} | T350V_L351Y_{—} | G236D_G237F_{—} | T350V_T366L_{—} |
| | | | L235F | F405A_Y407V | S239D_S267V_{—} | K392L_T394W |
| | | | | | H268D_Template1_{—} | |
| | | | | | D329*I | |
| 31524 | 29715 | Azym | L234F_G236N_{—} | T350V_L351Y_{—} | G236D_S239D_{—} | T350V_T366L_{—} |
| | | | H268Q_A327G_{—} | F405A_Y407V | V266L_S267A_{—} | K392L_T394W |
| | | | A330K_P331S_{—} | | H268D_G237D | |
| | | | P329I | | | |
| 31525 | 29724 | Azym | G236N_G237D | T350V_L351Y_{—} | G236D_G237F_{—} | T350V_T366L_{—} |
| | | | | F405A_Y407V | S239D_S267V_{—} | K392L_T394W |
| | | | | | H268D_Template7_{—} | |
| | | | | | E328*H_E329*R_{—} | |
| | | | | | A331*BY | |
| 31526 | 29689 | K/H | G236N_G237A_{—} | Y349C_T366S_{—} | G236D_G237F_{—} | S354C_T366W |
| | | | L235F | L368A_Y407V | S239D_S267V_{—} | |
| | | | | | H268D_Template1_{—} | |
| | | | | | D329*I | |
| 31527 | 29715 | K/H | L234F_G236N_{—} | Y349C_T366S_{—} | G236D_S239D_{—} | S354C_T366W |
| | | | H268Q_A327G_{—} | L368A_Y407V | V266L_S267A_{—} | |
| | | | A330K_P331S_{—} | | H268D_G237D | |
| | | | P329I | | | |
| 31528 | 29724 | K/H | G236N_G237D | Y349C_T366S_{—} | G236D_G237F_{—} | S354C_T366W |
| | | | | L368A_Y407V | S239D_S267V_{—} | |
| | | | | | H268D_Template7_{—} | |
| | | | | | E328*H_E329*R_{—} | |
| | | | | | A331*BY | |
| 31529 | 29689 | E/S | G236N_G237A_{—} | K392D_K409D | G236D_G237F_{—} | E356K_D399K |
| | | | L235F | | S239D_S267V_{—} | |
| | | | | | H268D_Template1_{—} | |
| | | | | | D329*I | |
| 31530 | 29715 | E/S | L234F_G236N_{—} | K392D_K409D | G236D_S239D_{—} | E356K_D399K |
| | | | H268Q_A327G_{—} | | V266L_S267A_{—} | |
| | | | A330K_P331S_{—} | | H268D_G237D | |
| | | | P329I | | | |
| 31531 | 29724 | E/S | G236N_G237D | K392D_K409D | G236D_G237F_{—} | E356K_D399K |
| | | | | | S239D_S267V_{—} | |
| | | | | | H268D_Template7_{—} | |
| | | | | | E328*H_E329*R_{—} | |
| | | | | | A331*BY | |
| 31532 | 29689 | Azym | G236D_G237F_{—} | T350V_L351Y_{—} | G236N_G237A_{—} | T350V_T366L_{—} |
| | | | S239D_S267V_{—} | F405A_Y407V | L235F | K392L_T394W |
| | | | H268D_Template1_{—} | | | |
| | | | D329*I | | | |
| 31533 | 29689 | E/S | G236D_G237F_{—} | K392D_K409D | G236N_G237A_{—} | E356K_D399K |
| | | | S239D_S267V_{—} | | L235F | |
| | | | H268D_Template1_{—} | | | |
| | | | D329*I | | | |
| 31534 | 29689 | K/H | G236D_G237F_{—} | Y349C_T366S_{—} | G236N_G237A_{—} | S354C_T366W |
| | | | S239D_S267V_{—} | L368A_Y407V | L235F | |
| | | | H268D_Template1_{—} | | | |
| | | | D329*I | | | |

### 2. Expression

Variants were prepared by site-directed mutagenesis and/or restriction/ligation using standard methods in a full-size antibody (FSA) scaffold based on trastuzumab with a heterodimeric IgG1 Fc comprising the mutations noted above and shown in Table 12.1.

All variants were expressed as described in the General Methods (Protocol 1) on a 50 mL scale, except for v01509, which was expressed on a 200 mL scale. The Protein A purification yield for each of the variants is shown in Table 12.2.

TABLE 12.2
Yields after Protein A Purification

| Variant # | Concentration (mg/ml) | Yield (mg/L) |
|---|---|---|
| v31509 | 7.00 | 68.9 |
| v31521 | 2.13 | 68.2 |
| v31522 | 2.18 | 70 |
| v31523 | 2.29 | 73.2 |
| v31524 | 2.00 | 64 |
| v31525 | 2.05 | 65.6 |
| v31526 | 2.18 | 69.8 |
| v31527 | 1.88 | 60.2 |
| v31528 | 1.75 | 56.2 |
| v31529 | 1.83 | 58.6 |
| v31530 | 2.18 | 69.8 |
| v31531 | 2.11 | 67.6 |
| v31532 | 2.22 | 71.2 |
| v31533 | 2.03 | 65 |
| v31534 | 2.21 | 70.8 |

All variants expressed at similar yields, indicating that there is no significant impact of the FcγRIIb selectivity-enhancing mutations on the expression yield, regardless of the heterodimer scaffold used.

### 3. FcγR Binding

Binding of each of the variants to the FcγRs was measured by SPR as described in the General Methods (Protocol 1). The results are shown in Table 12.3.

TABLE 12.3
FcγR Binding

| | | | | | | | Fold | | |
|---|---|---|---|---|---|---|---|---|---|
| | | K_{D} (M) | | | | | Increase¹ | | IIb/IIaR |
| Variant # | Heterodimer | FcγRI | FcγRIIIaV | FcγRIIaH | FcγRIIaR | FcγRIIb | FcγRIIaR | FcγRIIb | Selectivity² |
| 31509 | Azym | 6.7E−11 | 1.19E−06 | 1.15E−06 | 1.52E−06 | 4.20E−06 | 1.0 | 1.0 | 1.0 |
| 31521 | K/H | 9.8E−11 | 1.89E−06 | 1.90E−06 | 2.59E−06 | 7.74E−06 | 1.0 | 1.0 | 1.0 |
| 31522 | E/S | 7.1E−11 | 1.60E−06 | 1.53E−06 | 2.09E−06 | 6.34E−06 | 1.0 | 1.0 | 1.0 |
| 31523 | Azym | 1.5E−07 | NB³ | NB | 6.37E−07 | 1.56E−08 | 2.4 | 268.9 | 113.1 |
| 31524 | Azym | 5.2E−09 | NB | NB | 1.59E−06 | 6.53E−08 | 1.0 | 64.3 | 67.3 |
| 31525 | Azym | 1.7E−07 | NB | NB | 2.45E−06 | 1.37E−07 | 0.6 | 30.6 | 49.5 |
| 31526 | K/H | 2.4E−07 | 4.35E−06 | NB | 1.09E−06 | 3.62E−08 | 2.4 | 213.8 | 89.6 |
| 31527 | K/H | 7.9E−09 | NB | NB | 1.81E−06 | 8.09E−08 | 1.4 | 95.7 | 66.9 |
| 31528 | K/H | 2.3E−07 | NB | NB | 3.42E−06 | 7.32E−07 | 0.8 | 10.6 | 13.9 |
| 31529 | E/S | 1.9E−07 | NB | NB | 4.69E−07 | 2.07E−08 | 4.5 | 306.8 | 68.8 |
| 31530 | E/S | 6.8E−09 | NB | 2.64E−05 | 9.99E−07 | 6.02E−08 | 2.1 | 105.2 | 50.3 |
| 31531 | E/S | 1.9E−07 | NB | NB | 2.35E−06 | 8.60E−07 | 0.9 | 7.4 | 8.3 |
| 31532 | Azym | 1.5E−07 | NB | NB | 3.61E−07 | 1.39E−08 | 4.2 | 301.8 | 71.8 |
| 31533 | K/H | 1.9E−07 | NB | NB | 5.62E−07 | 2.56E−08 | 3.7 | 247.5 | 66.5 |
| 31534 | E/S | 2.2E−07 | NB | NB | 5.28E−07 | 2.75E−08 | 4.9 | 281.5 | 57.3 |

¹Fold increase in affinity over parental scaffold
²Selectivity is defined as IIb-Fold/IIaR-Fold
³NB = no binding

Variants v31523, v31526 and v31529, which comprise the CH2 mutations from original Strategy 1 variant v29689, showed a high level of selectivity ranging between 60-fold and 110-fold across the different heterodimer scaffolds. As the reported selectivity was calculated by taking four independent measurements (parental affinity to FcγRIIb, parental affinity to FcγRIIaR, variant affinity to FcγRIIb, and variant affinity to FcγRIIaR), with each measurement having an error margin, it can be concluded that the selectivity imparted by the CH2 mutations of variant v29689 is transferable across the heterodimeric scaffolds, within the error of the measurements. In addition, the results for binding of the variants v31532-v31534 indicate that this transferability is independent of the orientation of the CH2 mutations with respect to the CH3 mutations.

Variants v31524, v31527 and v31530, which comprise the CH2 mutations from original Strategy 2 variant v29715, also showed a high level of selectivity ranging between 50-fold and 70-fold across the different heterodimer scaffolds. Hence, it can be concluded that the selectivity imparted by the CH2 mutations of variant v29689 is likewise transferable across the heterodimeric scaffolds, within the error of the measurements.

Variants v31525, v31528 and v31531, which comprise the CH2 mutations from original Strategy 3 variant v29724, showed a high level of selectivity in the Azym heterodimeric scaffold (˜50-fold). For the K/H and E/S scaffolds only a modest ˜10-fold selectivity was observed. In the case of the E/S scaffold, however, LCMS determined high levels of homodimers which likely affected the level of selectivity (see below).

### 4. Heterodimer Purity

The heterodimer purity of selected variants was determined by liquid chromatography-mass spectrometry (LC-MS) as follows.

Variant samples were first de-glycosylated. As the variant samples contained Fc N-linked glycans only, samples were treated with a single enzyme, N-glycosidase F (PNGase-F; Sigma-Aldrich Co.) as follows: 0.1U PNGaseF/μg of antibody in 50 mM Tris-HCl pH 7.0, overnight incubation at 37° C., final protein concentration of 0.48 mg/mL. After de-glycosylation, the samples were stored at 4° C. prior to LC-MS analysis.

The de-glycosylated protein samples were analyzed by intact LC-MS using an Agilent 1100 HPLC system coupled to an LTQ-Orbitrap™ XL 9 mass spectrometer (ThermoFisher, Waltham, MA) (tuned for optimal detection of larger proteins (>50 kDa)) via an Ion Max electrospray source. The samples were injected onto a 2.1×30 mm Poros™ R2 reverse phase column (Applied Biosystems, Foster City, CA) and resolved using a 0.1% formic acid aq/acetonitrile (degassed) linear gradient consisting of increasing concentration (20-90%) of acetonitrile. The column was heated to 82.5° C. and solvents were heated pre-column to 80° C. to improve protein peak shape. The cone voltage (source fragmentation setting) was approximately 40 V, the FT resolution setting was 7,500 and the scan range was m/z 400-4,000. The LC-MS system was evaluated for IgG sample analysis using a de-glycosylated IgG standard (Waters IgG standard) as well as a de-glycosyated mAb standard mix (25:75 half:full sized mAb). For each LC-MS analysis, the mass spectra acquired across the antibody peak (typically 3.6-4.3 minutes) were summed and the entire multiply charged ion envelope (m/z 1,400-4,000) was deconvoluted into a molecular weight profile using the MaxEnt 1 module of MassLynx™, the instrument control and data analysis software (Waters, Milford, MA). The apparent amount of each antibody species in each sample was determined from peak heights in the resulting molecular weight profiles.

The results are shown in Table 12.4.

TABLE 12.4
LCMS Analysis of Heterodimer Purity

| | | Homodimer Species | | Half-Antibody Species | | |
|---|---|---|---|---|---|---|
| | Heterodimer | Higher- | Lower- | Higher- | Lower- | Other |
| Variant # | Species | Mass | Mass | Mass | Mass | Species |
| v31523 | 93% | 0% | 0% | 5% | 0% | 2% |
| v31524 | 91% | 2% | 0% | 3% | 1% | 2% |
| v31525 | 92% | 1% | 0% | 3% | 1% | 3% |
| v31526 | 98% | 0% | 0% | 0% | 0% | 1% |
| v31527 | 98% | 0% | 0% | 0% | 0% | 2% |
| v31528 | 97% | 0% | 0% | 0% | 0% | 3% |
| v31529 | 90% | 0% | 2% | 1% | 3% | 3% |
| v31530 | 94% | 0% | 0% | 0% | 3% | 4% |
| v31531 | 66% | 0% | 5% | 0% | 25% | 3% |

For all variants, the desired heterodimer was the most abundant species. Small amounts of homodimer and/or half-antibody were also detected. Only variant v31531 showed a large amount of half-antibody.

In all samples, the “other species” detected were primarily H1-H2 dimer (with no light chain). H1-H1 dimer was also detected in variants v31529 and v31531, as were smaller quantities of H2-H2 dimer in variants v31524 and v31525.

No significant side peaks were observed nor any evidence of remaining glycosylation in any of the variants.

### Example 13: Additional Modifications to LVG2

As shown in Table 11.1, some of the LVG2 variants showed increased binding to C1q. Additional combinations of mutations identified in the preceding Examples as being FcγRIIb selectivity-enhancing were tested with the goal of finding new variants that retained FcγRIIb selectivity, without increasing binding to C1q.

The strategy employed in attempting to decrease the affinity of the variants for C1q was to include mutations in the lower hinge region (positions 233-237) that had already been tested and shown to preserve a high level of FcγRIIb selectivity (see Example 6). The following three approaches were adopted:
1. Combine mutations in the lower hinge region of chain A with chain B mutations from Strategy 1 (Table 6.17) that showed the highest FcγRIIb selectivity.
2. Combine mutations in the lower hinge region of chain A with chain B mutations from Strategy 2 (Table 6.18) that showed the highest FcγRIIb selectivity.
3. Combine mutations in the lower hinge region of chain A with chain B mutations from Strategy 3 (Table 6.19) that showed the highest FcγRIIb selectivity.

### Approach 1

Analysis of the chain B mutations that had the highest level of selectivity from Strategy 1 designs identified the mutations D329*I and G330*K.

Two options for mutations that could be combined with D329*I in chain B were identified: I332L or F328*Y.

The following criteria were used to select chain A mutations to combine with the D329*I_I332L chain B mutations:
“IIb Selectivity Fold wrt Control”>1.2
“IIb-Fold wrt Control”>0.1
T-cell epitope score <15 (calculated using an in silico prediction tool)
Exclude Met, Trp

Combining chain A mutations that met the above criteria with the D329*I_I332L chain B mutations resulted in a total of 36 new variants (see Table 13.1).

The following criteria were used to select chain A mutations to combine with the F328*Y_D329*I chain B mutations:
“IIb Selectivity Fold wrt Control”>1.6
“IIb-Fold wrt Control”>0.1
T-cell epitope score <15 (calculated using an in silico prediction tool)
Exclude Met, Trp
For position L235, include only L325F for aromatics at this position

Combining chain A mutations that met the above criteria with the F328*Y_D329*I chain B mutations resulted in a total of 12 new variants (see Table 13.1).

The following criteria were used to select chain A mutations to combine with the G330*K chain B mutation:
“IIb Selectivity Fold wrt Control”>1.5
“IIb-Fold wrt Control”>0.1
T-cell epitope score <15 (calculated using an in silico prediction tool)
Exclude Met, Trp

Combining chain A mutations that met the above criteria with the G330*K chain B mutation resulted in a total of 13 new variants (see Table 13.1).

### Approach 2

For Strategy 2-based designs, the mutation G237L was selected for combination with chain A mutations. Inclusion of G237L should reduce potential liabilities arising from the D237-P238 motif.

The following criteria were used to select chain A mutations to combine with the G237L chain B mutation:
“IIb Selectivity Fold wrt Control”>1.5
“IIb-Fold wrt Control”>0.1

Combining chain A mutations that met the above criteria with the G237L chain B mutation resulted in a total of 12 new variants (see Table 13.1). The mutation E269W, which also met the above criteria, was excluded as including an exposed tryptophan residue is undesirable.

### Approach 3

Analysis of the chain B mutations that had the highest level of selectivity from Strategy 3 designs identified Template 7 as the alternative loop template that showed the best improvement in FcγRIIb selectivity.

Criteria that were followed in order to select chain A mutations to combine with the Template 7 in chain B:
“IIb Selectivity Fold wrt Control”>1.6
“IIb-Fold wrt Control”>0.1
T-cell epitope score <15 (calculated using an in silico prediction tool)
Exclude A237D (included in controls), Trp

Combining chain A mutations that met the above criteria with Template 7 in chain B resulted in a total of 10 new variants (see Table 13.1).

Variants were constructed in the trastuzumab full-size antibody (FSA) scaffold (Scaffold 3) and tested for FcγR binding, C1q binding and thermal stability (DSF) as described in the General Methods. The variants were also tested for stability at low pH as described in the General Methods.

The results are shown in Table 13.1.

TABLE 13.1
Characteristics of Modified LVG2 Variants

| | | | | | | | Change | Change | |
|---|---|---|---|---|---|---|---|---|---|
| | | | | | | | in | in | |
| | | | | | | C1q | HMWS | LMWS | |
| | Chain A | Chain B | IIb- | IIaR- | IIb | Binding | low pH | low pH | |
| Variant # | Mutations¹ | Mutations¹ | fold² | fold² | Selectivity³ | (% WT) | (%)⁴ | (%)⁴ | ΔTm⁵/° C. |
| 21653 | FSA WT | FSA WT | 1 | 1 | 1 | 100 | −1 | 0 | 0 |
| 22126 | L234D_{—} | Template1_{—} | 25 | 2 | 13 | 34 | 2 | 2 | |
| | G236N | G236D_S239D_{—} | | | | | | | |
| | | S267I_H268D | | | | | | | |
| 22127 | L234D_G236N_{—} | G236D_S239D_{—} | 49 | 4 | 12 | 215 | 12 | −2 | |
| | S267A | V266L_S267A_{—} | | | | | | | |
| | | H268D | | | | | | | |
| 22128 | L234F_G236N_{—} | L234F_G236D_{—} | 33 | 4 | 9 | 3 | 2 | 2 | |
| | H268Q_K274Q_{—} | S239D_V266L_{—} | | | | | | | |
| | A327G_A330K_{—} | S267A_H268D_{—} | | | | | | | |
| | P331S | K274Q_A327G_{—} | | | | | | | |
| | | A330S_P331S | | | | | | | |
| 31186 | A237D_strat1 | D329*I_strat1 | 97 | 1 | 76 | 91 | 7 | 2 | −15 |
| 31187 | L235F_strat1 | D329*I_strat1 | 542 | 7 | 77 | 240 | 3 | 2 | −11 |
| 31188 | L235F_strat1 | G330*K_strat1 | 1,858 | 42 | 44 | 241 | 4 | 0 | −11 |
| 31190 | L235D_strat2 | G237L_strat2 | 187 | 7 | 26 | 2 | 1 | 4 | −10 |
| 31191 | A237D_strat1 | template7_{—} | 35 | 1 | 44 | 27 | 2 | 3 | −8 |
| | | E328*H_E329*R_{—} | | | | | | | |
| | | A331*BY_{—} | | | | | | | |
| | | strat3 | | | | | | | |
| 31192 | L235D_strat2 | D329*I_strat1 | 260 | 5 | 53 | 6 | 4 | 1 | −14 |
| 31209 | L235F_strat1 | D329*I_F328*Y_{—} | 617 | 8 | 79 | 251 | 2 | 3 | −11 |
| | | strat1 | | | | | | | |
| 31211 | L235F_strat1_{—} | D329*I_strat1 | 105 | 2 | 67 | 131 | 8 | 2 | −15 |
| | A237D | | | | | | | | |
| 31213 | L235F_strat1 | D329*I_I332L_{—} | 732 | 8 | 95 | 232 | 4 | 2 | −12 |
| | | strat1 | | | | | | | |
| 31256 | P329I_strat2 | G237D_strat2 | 121 | 4 | 34 | 3 | 1 | 3 | −14 |
| 32210 | A237D_strat1 | D329*I_I332L_{—} | 121 | 1 | 94 | 76 | 11 | 2 | −16 |
| | | strat1 | | | | | | | |
| 32211 | A237E_strat1 | D329*I_I332L_{—} | 42 | 1 | 50 | 92 | 4 | 2 | −14 |
| | | strat1 | | | | | | | |
| 32212 | A237G_strat1 | D329*I_I332L_{—} | 1,328 | 21 | 62 | 226 | 4 | 2 | −12 |
| | | strat1 | | | | | | | |
| 32213 | A237L_strat1 | D329*I_I332L_{—} | 121 | 2 | 68 | 120 | 4 | 3 | −12 |
| | | strat1 | | | | | | | |
| 32214 | A237N_strat1 | D329*I_I332L_{—} | 129 | 2 | 62 | 126 | 4 | 3 | −13 |
| | | strat1 | | | | | | | |
| 32215 | A237Q_strat1 | D329*I_I332L_{—} | | 1 | | 89 | 2 | 2 | −11 |
| | | strat1 | | | | | | | |
| 32216 | L234D_strat1 | D329*I_I332L_{—} | 342 | 5 | 72 | 114 | 7 | 2 | −13 |
| | | strat1 | | | | | | | |
| 32217 | L234H_strat1 | D329*I_I332L_{—} | 198 | 3 | 59 | 171 | 2 | 3 | −11 |
| | | strat1 | | | | | | | |
| 32218 | L234K_strat1 | D329*I_I332L_{—} | 78 | 1 | 57 | 95 | 3 | 2 | −10 |
| | | strat1 | | | | | | | |
| 32219 | L234N_strat1 | D329*I_I332L_{—} | | 4 | | 169 | 4 | 1 | −11 |
| | | strat1 | | | | | | | |
| 32220 | L234P_strat1 | D329*I_I332L_{—} | 168 | 3 | 61 | 121 | 4 | 2 | −11 |
| | | strat1 | | | | | | | |
| 32221 | L234Q_strat1 | D329*I_I332L_{—} | 216 | 3 | 64 | 150 | 4 | 3 | −11 |
| | | strat1 | | | | | | | |
| 32222 | L234S_strat1 | D329*I_I332L_{—} | 223 | 4 | 59 | 162 | 3 | 3 | −11 |
| | | strat1 | | | | | | | |
| 32223 | L234T_strat1 | D329*I_I332L_{—} | 242 | 4 | 67 | 165 | 3 | 2 | −11 |
| | | strat1 | | | | | | | |
| 32224 | L234V_strat1 | D329*I_I332L_{—} | 230 | 3 | 66 | 162 | 2 | 2 | −12 |
| | | strat1 | | | | | | | |

| | | | | | | | Change | Change | |
|---|---|---|---|---|---|---|---|---|---|
| | | | | | | | in | in | |
| | | | | | | C1q | HMWS | LMWS | |
| | Chain A | Chain B | IIb- | IIaR- | IIb | Binding | low pH | low pH | |
| Variant # | Mutations¹ | Mutations¹ | fold² | fold² | Selectivity³ | (% WT) | (%)⁴ | (%)⁴ | ΔTm⁵/° C. |
| 32225 | L235A_strat1 | D329*I_I332L_{—} | 193 | 3 | 75 | 125 | 4 | 5 | −11 |
| | | strat1 | | | | | | | |
| 32226 | L235D_strat1 | D329*I_I332L_{—} | 287 | 3 | 84 | 94 | 10 | 0 | −13 |
| | | strat1 | | | | | | | |
| 32227 | L235E_strat1 | D329*I_I332L_{—} | 233 | 3 | 70 | 94 | 4 | 1 | −13 |
| | | strat1 | | | | | | | |
| 32228 | L235I_strat1 | D329*I_I332L_{—} | 226 | 4 | 65 | 106 | 12 | −3 | −11 |
| | | strat1 | | | | | | | |
| 32229 | L235T_strat1 | D329*I_I332L_{—} | 160 | 3 | 62 | 108 | 2 | 2 | −11 |
| | | strat1 | | | | | | | |
| 32230 | L235V_strat1 | D329*I_I332L_{—} | 207 | 3 | 72 | 123 | 3 | 2 | −11 |
| | | strat1 | | | | | | | |
| 32231 | L235Y_strat1 | D329*I_I332L_{—} | 831 | 10 | 87 | 229 | 3 | 2 | −12 |
| | | strat1 | | | | | | | |
| 32232 | N236D_strat1 | D329*I_I332L_{—} | 365 | 6 | 58 | 130 | 6 | 2 | −14 |
| | | strat1 | | | | | | | |
| 32233 | N236F_strat1 | D329*I_I332L_{—} | 476 | 8 | 60 | 207 | 3 | 1 | −12 |
| | | strat1 | | | | | | | |
| 32234 | N236I_strat1 | D329*I_I332L_{—} | 338 | 6 | 56 | 203 | 4 | 0 | −12 |
| | | strat1 | | | | | | | |
| 32235 | N236T_strat1 | D329*I_I332L_{—} | 191 | 4 | 44 | 185 | 3 | 2 | −12 |
| | | strat1 | | | | | | | |
| 32236 | N236Y_strat1 | D329*I_I332L_{—} | 499 | 9 | 57 | 208 | 3 | 2 | −13 |
| | | strat1 | | | | | | | |
| 32237 | S239A_strat1 | D329*I_I332L_{—} | 262 | 4 | 59 | 174 | 3 | 1 | −13 |
| | | strat1 | | | | | | | |
| 32238 | S239D_strat1 | D329*I_I332L_{—} | 178 | 4 | 50 | 155 | 4 | 1 | −14 |
| | | strat1 | | | | | | | |
| 32239 | S239G_strat1 | D329*I_I332L_{—} | 298 | 4 | 68 | 150 | 4 | 1 | −14 |
| | | strat1 | | | | | | | |
| 32240 | S239H_strat1 | D329*I_I332L_{—} | 200 | 3 | 63 | 134 | 3 | 3 | −13 |
| | | strat1 | | | | | | | |
| 32241 | S239N_strat1 | D329*I_I332L_{—} | 355 | 5 | 74 | 148 | 20 | 3 | −13 |
| | | strat1 | | | | | | | |
| 32242 | S239P_strat1 | D329*I_I332L_{—} | 24 | 1 | 49 | 2 | 11 | 0 | −17 |
| | | strat1 | | | | | | | |
| 32243 | S239Q_strat1 | D329*I_I332L_{—} | 219 | 4 | 56 | 157 | 1 | 1 | −10 |
| | | strat1 | | | | | | | |
| 32244 | S239T_strat1 | D329*I_I332L_{—} | 345 | 5 | 71 | 159 | 5 | 1 | −12 |
| | | strat1 | | | | | | | |
| 32245 | A237D_strat1 | D329*I_F328*Y_{—} | 98 | 2 | 59 | 122 | 7 | 2 | −15 |
| | | strat1 | | | | | | | |
| 32246 | A237L_strat1 | D329*I_F328*Y_{—} | 128 | 2 | 56 | 148 | 2 | 2 | −10 |
| | | strat1 | | | | | | | |
| 32247 | A237N_strat1 | D329*I_F328*Y_{—} | 159 | 3 | 58 | 162 | 3 | 1 | −11 |
| | | strat1 | | | | | | | |
| 32248 | L234D_strat1 | D329*I_F328*Y_{—} | 328 | 6 | 56 | 156 | 6 | 1 | −11 |
| | | strat1 | | | | | | | |
| 32251 | L235Y_strat1 | D329*I_F328*Y_{—} | 911 | 11 | 80 | 235 | 2 | 2 | −10 |
| | | strat1 | | | | | | | |
| 32252 | S239A_strat1 | D329*I_F328*Y_{—} | 349 | 7 | 54 | 205 | 2 | 2 | −11 |
| | | strat1 | | | | | | | |
| 32253 | S239G_strat1 | D329*I_F328*Y_{—} | 242 | 5 | 51 | 176 | 4 | 3 | −13 |
| | | strat1 | | | | | | | |
| 32254 | S239H_strat1 | D329*I_F328*Y_{—} | 159 | 3 | 47 | 159 | 2 | 3 | −11 |
| | | strat1 | | | | | | | |
| 32255 | S239T_strat1 | D329*I_F328*Y_{—} | 344 | 6 | 57 | 194 | 3 | 2 | −11 |
| | | strat1 | | | | | | | |
| 32256 | A237D_strat1 | G330*K_strat1 | 202 | 6 | 35 | 124 | 6 | 1 | −14 |
| 32257 | A237E_strat1 | G330*K_strat1 | 141 | 6 | 24 | 145 | 2 | 1 | −12 |
| 32258 | A237L_strat1 | G330*K_strat1 | 476 | 11 | 44 | 201 | 2 | 0 | −10 |
| 32259 | A237N_strat1 | G330*K_strat1 | 452 | 14 | 33 | 192 | 3 | 0 | −11 |
| 32260 | L234D_strat1 | G330*K_strat1 | 1,263 | 34 | 37 | 189 | 6 | −2 | −11 |
| 32261 | L234Q_strat1 | G330*K_strat1 | 683 | 21 | 32 | 209 | 4 | 1 | −10 |
| 32262 | L234T_strat1 | G330*K_strat1 | 697 | 22 | 31 | 210 | 2 | 1 | −10 |
| 32263 | L235A_strat1 | G330*K_strat1 | 396 | 13 | 30 | 170 | 4 | 1 | −10 |
| 32264 | L235D_strat1 | G330*K_strat1 | 557 | 17 | 33 | 146 | 12 | −5 | −11 |
| 32265 | S239A_strat1 | G330*K_strat1 | 1,120 | 36 | 31 | 224 | 2 | 2 | −11 |
| 32266 | S239G_strat1 | G330*K_strat1 | 1,154 | 30 | 38 | 219 | 1 | 1 | −13 |
| 32267 | S239H_strat1 | G330*K_strat1 | 803 | 25 | 33 | 201 | 4 | 0 | −11 |
| 32268 | S239T_strat1 | G330*K_strat1 | 1,658 | 39 | 43 | 210 | 6 | −2 | −11 |
| 32270 | E272Y_strat2 | G237L_strat2 | 196 | 11 | 18 | −1 | 1 | 1 | −11 |
| 32271 | G237L_strat2 | G237L_strat2 | 251 | 10 | 26 | 3 | 1 | 1 | −9 |
| 32272 | K330T_strat2 | G237L_strat2 | 164 | 9 | 18 | 3 | 1 | 1 | −10 |
| 32273 | L235R_strat2 | G237L_strat2 | 42 | 2 | 18 | 2 | 1 | 2 | −8 |
| 32274 | P329A_strat2 | G237L_strat2 | 95 | 4 | 26 | 2 | 1 | 0 | −9 |
| 32275 | P329I_strat2 | G237L_strat2 | 97 | 4 | 25 | 4 | 1 | 3 | −10 |
| 32276 | P329V_strat2 | G237L_strat2 | 94 | 4 | 24 | 3 | 1 | 3 | −10 |
| 32277 | S239G_strat2 | G237L_strat2 | 260 | 10 | 25 | 0 | 1 | 2 | −12 |
| 32278 | S267A_strat2 | G237L_strat2 | 155 | 9 | 17 | 1 | 1 | 2 | −10 |
| 32279 | S267K_strat2 | G237L_strat2 | 53 | 3 | 19 | 1 | 1 | 2 | −9 |
| 32280 | A237L_strat1 | template7_{—} | 39 | 1 | 31 | 70 | 0 | 3 | −4 |
| | | E328*H_E329*R_{—} | | | | | | | |
| | | A331*BY_{—} | | | | | | | |
| | | strat3 | | | | | | | |
| 32281 | A237N_strat1 | template7_{—} | 43 | 1 | 34 | 50 | 1 | 3 | −5 |
| | | E328*H_E329*R_{—} | | | | | | | |
| | | A331*BY_{—} | | | | | | | |
| | | strat3 | | | | | | | |
| 32282 | L234D_strat1 | template7_{—} | 89 | 2 | 36 | 31 | 0 | 1 | −4 |
| | | E328*H_E329*R_{—} | | | | | | | |
| | | A331*BY_{—} | | | | | | | |
| | | strat3 | | | | | | | |
| 32283 | L234Q_strat1 | template7_{—} | 72 | 2 | 37 | 57 | 0 | 2 | −3 |
| | | E328*H_E329*R_{—} | | | | | | | |
| | | A331*BY_{—} | | | | | | | |
| | | strat3 | | | | | | | |
| 32284 | L235D_strat1 | template7_{—} | 80 | 2 | 40 | 18 | −3 | 2 | −5 |
| | | E328*H_E329*R_{—} | | | | | | | |
| | | A331*BY_{—} | | | | | | | |
| | | strat3 | | | | | | | |
| 32285 | L235F_strat1 | template7_{—} | 147 | 4 | 34 | 158 | 0 | 1 | −4 |
| | | E328*H_E329*R_{—} | | | | | | | |
| | | A331*BY_{—} | | | | | | | |
| | | strat3 | | | | | | | |
| 32286 | S239A_strat1 | template7_{—} | 81 | 2 | 33 | 69 | 0 | 2 | −5 |
| | | E328*H_E329*R_{—} | | | | | | | |
| | | A331*BY_{—} | | | | | | | |
| | | strat3 | | | | | | | |
| 32287 | S239G_strat1 | template7_{—} | 76 | 2 | 35 | 53 | 0 | 2 | −6 |
| | | E328*H_E329*R_{—} | | | | | | | |
| | | A331*BY_{—} | | | | | | | |
| | | strat3 | | | | | | | |
| 32288 | S239H_strat1 | template7_{—} | 54 | 1 | 36 | 39 | 0 | 2 | −5 |
| | | E328*H_E329*R_{—} | | | | | | | |
| | | A331*BY_{—} | | | | | | | |
| | | strat3 | | | | | | | |
| 32289 | S239T_strat1 | template7_{—} | 97 | 3 | 36 | 59 | 0 | 3 | −4 |
| | | E328*H_E329*R_{—} | | | | | | | |
| | | A331*BY_{—} | | | | | | | |
| | | strat3 | | | | | | | |
| 32291 | S267H_strat2 | G237L_strat2 | 132 | 6 | 23 | 4 | 1 | 2 | −10 |
| 32292 | L235D_strat2 | D329*I_I332L_{—} | 451 | 6 | 78 | 2 | 6 | 2 | −15 |
| | | strat1 | | | | | | | |
| 32293 | S267A_strat2 | D329*I_I332L_{—} | 323 | 6 | 58 | 2 | 3 | 2 | −14 |
| | | strat1 | | | | | | | |
| 32294 | K330T_strat2 | D329*I_I332L_{—} | 339 | 5 | 68 | 4 | 5 | 1 | −14 |
| | | strat1 | | | | | | | |
| 32295 | P329I_strat2 | D329*I_I332L_{—} | 103 | 1 | 82 | −1 | 8 | 0 | −15 |
| | | strat1 | | | | | | | |
| 32296 | A237E_strat1 | template7_{—} | 15 | 1 | 29 | 12 | 0 | 2 | −5 |
| | | E328*H_E329*R_{—} | | | | | | | |
| | | A331*BY_{—} | | | | | | | |
| | | strat3 | | | | | | | |
| v12 | v12_FSA | v12_FSA | 117 | 1 | 79 | 3 | 2 | 2 | −8 |

¹Mutation notation is in the format “A237D_strat1,” where “A237D” indicates the mutation made with “A” representing the parental residue being replaced, “237” representing the position and “D” representing the replacement residue, and “strat1” specifies the parental CH2 mutations (i.e. those of Launching Module 1). “strat2” refers to the mutations of Launching Module 2.
²Fold change in affinity over wild-type
³Selectivity is defined as IIb-Fold/IIaR-Fold
⁴HMWS = high molecular weight species; LMWS = low molecular weight species; % change over amounts at neutral pH
⁵Compared to wild-type (WT)

All tested variants retained an FcγRIIb selectivity that was significantly higher than wild-type, with some variants also showing an increase in selectivity over their parental variant. C1q binding was decreased for some variants. Thermal stability for the tested variants remained in a similar range to that of the respective parental variants.

The values “Change in HMWS low pH” and “Change in LMWS low pH” provide an indication of the stability of the variants under low pH conditions, such as during purification or production, or under suboptimal storage conditions. The HMWS values provide an indication of aggregate formation and the LMWS values provide an indication of fragmentation. For the purposes of ranking the variants, preferred values of less than 10% HMWS and less than 5% LMWS were employed.

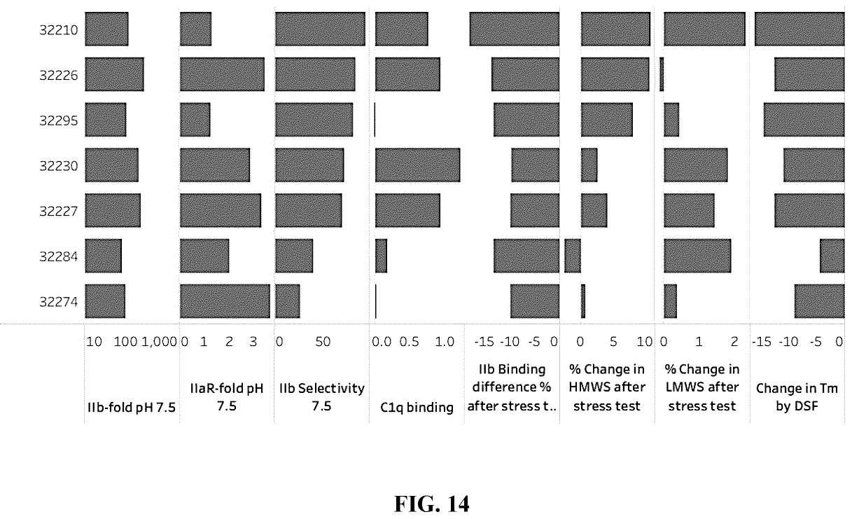

Variants v32210, v32226, v32295, v32230, v32227, v32274 and v32284 were selected for further study. Variants v32210, v32226, v32295, v32230 and v32227 showed the highest FcγRIIb selectivity of the tested variants, variant v32274 was the best performing Strategy 2-based variant and variant v32284 was the best performing Strategy 3-based variant. The experimental parameters for these variants are summarized in the plot shown in FIG. 14.

### Example 14: Transferability to Other Full-Size Antibodies

Selected modified LVG2 variants from Example 13 were constructed in the following full-size antibody (FSA) scaffolds: trastuzumab (anti-HER2; Scaffold 3), anti-CD19 (Scaffold 4) and an anti-CD40 scaffold. The anti-CD40 scaffold was based on the Chi Lob 7/4 anti-CD40 antibody (Johnson, et al., 2010, J Clin Oncology, 25 (15)_suppl 2507-2507) comprising the same heterodimeric Fc as for Scaffold 2.

FSA variants were tested for FcγR binding by SPR as described below.

Binding affinity for the FcγRs was measured by SPR using an IBIS MX96 SPR imaging system (IBIS Technologies, Enschede, The Netherlands) at 25° C. with HBS-EP+pH 7.4 as the running buffer. Sample was diluted in pH 4.5 acetate buffer then captured onto a SensEye® G Easy2Spot® sensor chip (SensEye, Enschede, The Netherlands) using a continuous flow microspotter (Carterra, Salt Lake City, UT). The receptor was diluted to a defined concentration range in HBS-EP+pH 7.4 buffer. Twelve concentrations (10 2-fold step dilutions from a highest concentration of 2048 nM plus 0 nM) were used per analyte at pH 7.4. The chip surface was regenerated after each analyte concentration injection with 10 mM glycine pH 3.0. Results were analysed using Scrubber V2 (BioLogic Software, Canberra, Australia) and a kinetic fit model.

The results are shown in Table 14.1.

TABLE 14.1
Comparison of FcγRIIb Binding for Variants with Fab Sequences that Target HER2, CD19 or CD40

| | Mutations | | | Affinity, Kd (M) | | IIb |
|---|---|---|---|---|---|---|
| Variant # | Chain A | Chain B | Target | FcγRIIb | FcγRIIaR | Selectivity¹ |
| 21653 | WT | WT | HER2 | 2.0E−06 | 6.3E−07 | 1.0 |
| | | | CD19 | 2.2E−06 | 7.2E−07 | 1.0 |
| | | | CD40 | 2.1E−06 | 5.6E−07 | 1.0 |
| 31188 | L235F_{—} | Template 1 (G330*K) + | HER2 | 2.7E−09 | 1.0E−08 | 11.7 |
| | G236N_G237A | G236D_G237F_S239D_{—} | CD19 | 3.0E−09 | 1.1E−08 | 10.8 |
| | | S267V_H268D | CD40 | 2.4E−09 | 8.9E−09 | 14.1 |
| 32227 | L235E_{—} | Template 1 (D329*I) + | HER2 | 9.0E−09 | 3.2E−07 | 115.5 |
| | G236N_G237A | G236D_G237F_S239D_{—} | CD19 | 8.9E−09 | 2.8E−07 | 97.5 |
| | | S267V_H268D_{—} | CD40 | 9.3E−09 | 3.0E−07 | 119.0 |
| | | I332L | | | | |
| 32274 | L234F_G236N_{—} | G236D_G237L_S239D_{—} | HER2 | 2.1E−08 | 1.9E−07 | 28.8 |
| | H268Q_A327G_{—} | V266L_S267A_{—} | CD19 | 1.6E−08 | 1.5E−07 | 28.6 |
| | P329A_A330K_{—} | H268D | CD40 | 1.4E−08 | 1.3E−07 | 33.3 |
| | P331S | | | | | |
| 32284 | L235D_{—} | Template 7 + | HER2 | 2.1E−08 | 5.6E−07 | 83.4 |
| | G236N_G237A | G236D_G237F_S239D_{—} | CD19 | 2.4E−08 | 2.7E−07 | 35.7 |
| | | S267V_H268D | CD40 | 2.3E−08 | 2.6E−07 | 41.9 |
| 32295 | L234F_G236N_{—} | Template 1 (D329*I) + | HER2 | 1.7E−08 | 7.1E−07 | 135.1 |
| | H268Q_A327G_{—} | G236D_G237F_S239D_{—} | CD19 | 1.5E−08 | 6.2E−07 | 125.4 |
| | P329I_A330K_{—} | S267V_H268D_{—} | CD40 | 1.7E−08 | 5.7E−07 | 122.9 |
| | P331S | I332L | | | | |
| v12² | E233D_G237D_{—} | E233D_G237D_{—} | HER2 | 1.3E−08 | 5.0E−07 | 123.5 |
| | P238D_H268D_{—} | P238D_H268D_{—} | CD19 | 1.3E−08 | 4.6E−07 | 105.9 |
| | P271G_A330R | P271G_A330R | CD40 | 1.0E−08 | 3.9E−07 | 140.0 |

¹Selectivity is defined as IIb-Fold/IIaR-Fold
²Mimoto, et al., 2013, Protein Eng. Des. Sel., 26: 589-598)

The results show that very similar levels of affinity and selectivity were observed in the different FSAs for all variants tested, including the control v12. This suggests that the mutations comprised by these variants are transferable across FSAs and that the Fab comprised by an FSA does not affect the engineered affinity and selectivity.

### Example 15: C1Q Binding and Complement Activation

Select variants in combination with 3 different Fabs from Example 14 were tested for C1q binding, and the same variants in combination with anti-CD40 Fabs were tested for complement-dependent cytotoxicity (CDC) activity. An Fc negative variant (“Neg” in the tables below; L234A, L235A, D265S) and control v12 were also included. The anti-CD20 antibody rituximab was included in the CDC assays as a positive control. Table 15.1 lists the variants and controls tested in this Example.

TABLE 15.1
Variants and Controls Tested

| Variant | | | |
|---|---|---|---|
| # | Chain A Mutations | Chain B Mutations | Loop Sequence |
| Neg | L234A_L235A_D265S | L234A_L235A_D265S | WT IgG1 |
| v12¹ | E233D_G237D_P238D | E233D_G237D_P238D_H268D_ | WT IgG1 |
| | H268D_P271G_A330R | P271G_A330R | |
| SELF² | S267E_L328F | S267E_L328F | WT IgG1 |
| 22096 | L234D_G236N | Template 66 + | DFDQNQGEVV |
| | | G236D_S239D_S267I_H268D | [SEQ ID NO: 12] |
| 26370 | G236N_G237A | Template 1 + G236D_ | STWFDGGYAT |
| | | G237F_S239D_S267V_H268D_P271V | [SEQ ID NO: 6] |
| 26774 | L234F_G236N_H268Q_ | G236D S239D V266L S267A H268D | WT IgG |
| | N325G_A327G_A330K_ | | |
| | P331S | | |
| 27092 | L234F_G236N_H268Q_ | G236D_S239D_V266L_S267A_H268D_ | WT IgG |
| | A327G_A330K_P331S | D270Y | |
| 31186 | G236N_G237D | Template 1 (D329*I) + | STWFIGGYAT |
| | | G236D_G237F_S239D_S267V_H268D | [SEQ ID NO: 47] |
| 31188 | L235F_G236N_G237A | Template 1 (G330*K) + | STWFDKGYAT |
| | | G236D_G237F_S239D_S267V_H268D | [SEQ ID NO: 68] |
| 31191 | G236N_G237D | Template 7 | GLDHRGKGYV |
| | | (E328*H_E329*R_A331*BY) + | [SEQ ID NO: 73] |
| | | G236D_G237F_S239D_S267V_H268D | |
| 31192 | L234F_L235D_G236N_ | Template 1 (D329*I) + | STWFIGGYAT |
| | H268Q_A327G_A330K_ | G236D_G237F_S239D_S267V_H268D | [SEQ ID NO: 47] |
| | P331S | | |
| 31213 | L235F_G236N_G237A | Template 1 (D329*I) + | STWFIGGYAT |
| | | G236D_G237F_S239D_S267V_H268D_ | [SEQ ID NO: 47] |
| | | I332L | |
| 32210 | G236N_G237D | Template 1 (D329*I) + | STWFIGGYAT |
| | | G236D_G237F_S239D_S267V_H268D_ | [SEQ ID NO: 47] |
| | | I332L | |
| 32211 | G236N_G237E | Template 1 (D329*I) + | STWFIGGYAT |
| | | G236D_G237F_S239D_S267V_H268D_ | [SEQ ID NO: 47] |
| | | I332L | |
| 32212 | G236N | Template 1 (D329*I) + | STWFIGGYAT |
| | | G236D_G237F_S239D_S267V_H268D_ | [SEQ ID NO: 47] |
| | | I332L | |
| 32226 | L235D_G236N_G237A | Template 1 (D329*I) + | STWFIGGYAT |
| | | G236D_G237F_S239D_S267V_H268D_ | [SEQ ID NO: 47] |
| | | I332L | |
| 32227 | L235E_G236N_G237A | Template 1 (D329*I) + | STWFIGGYAT |
| | | G236D_G237F_S239D_S267V_H268D_ | [SEQ ID NO: 47] |
| | | 1332L | |
| 32230 | L235V_G236N_G237A | Template 1 (D329*I) + | STWFIGGYAT |
| | | G236D_G237F_S239D_S267V_H268D_ | [SEQ ID NO: 47] |
| | | I332L | |
| 32231 | L235Y_G236N_G237A | Template 1 (D329*I) + | STWFIGGYAT |
| | | G236D_G237F_S239D_S267V_H268D_ | [SEQ ID NO: 47] |
| | | I332L | |
| 32242 | G236N_G237A_S239P | Template 1 (D329*I) + | STWFIGGYAT |
| | | G236D_G237F_S239D_S267V_H268D_ | [SEQ ID NO: 47] |
| | | I332L | |
| 32274 | L234F_G236N_H268Q | G236D_G237L_S239D_V266L_S267A_ | WT IgG |
| | A327G_P329A_A330K | H268D | |
| | P331S | | |
| 32282 | L234D_G236N_G237A | Template 7 | GLDHRGKGYV |
| | | (E328*H_E329*R_A331*BY) + | [SEQ ID NO: 73] |
| | | G236D_G237F_S239D_S267V_H268D | |
| 32284 | L235D_G236N_G237A | Template 7 | GLDHRGKGYV |
| | | (E328*H_E329*R_A331*BY) + | [SEQ ID NO: 73] |
| | | G236D_G237F_S239D_S267V_H268D | |
| 32287 | G236N_G237A_S239G | Template 7 | GLDHRGKGYV |
| | | (E328*H_E329*R_A331*BY) + | [SEQ ID NO: 73] |
| | | G236D_G237F_S239D_S267V_H268D | |
| 32288 | G236N_G237A_S239H | Template 7 | GLDHRGKGYV |
| | | (E328*H_E329*R_A331*BY) + | [SEQ ID NO: 73] |
| | | G236D_G237F_S239D_S267V_H268D | |
| 32292 | L234F_L235D_G236N_ | Template 1 (D329*I) + | STWFIGGYAT |
| | H268Q_A327G_A330K_ | G236D_G237F_S239D_S267V_H268D_ | [SEQ ID NO: 47] |
| | P331S | I332L | |
| 32293 | L234F_G236N_S267A_ | Template 1 (D329*I) + | STWFIGGYAT |
| | H268Q_A327G_A330K_ | G236D_G237F_S239D_S267V_H268D_ | [SEQ ID NO: 47] |
| | P331S | I332L | |
| 32294 | L234F_G236N_H268Q_ | Template 1 (D329*I) + | STWFIGGYAT |
| | A327G_A330T_P331S_ | G236D_G237F_S239D_S267V_H268D_ | [SEQ ID NO: 47] |
| | | I332L | |
| 32295 | L234F_G236N_H268Q_ | Template 1 (D329*I) + | STWFIGGYAT |
| | A327G_P3291_A330K_ | G236D_G237F_S239D_S267V_H268D_ | [SEQ ID NO: 47] |
| | P331S | I332L | |
| 32296 | G236N_G237E | Template 7 | GLDHRGKGYV |
| | | (E328*H_E329*R_A331*BY) + | [SEQ ID NO: 73] |
| | | G236D_G237F_S239D_S267V_H268D | |

¹Mimoto, et al., 2013, Protein Eng. Des. Sel., 26:589-598)
²Chu, et al., 2008, Mol. Immunol., 45:3926-3933

### In Vitro C1q Binding

The binding of the tested variants to C1q was measured by ELISA as described in the General Methods with the following modifications. Assays were conducted in Maxisorp™ 384-well immunoplates with all test article and reagent amounts reduced by a factor of 4. All washes were ×6. Plates were read at 450 nm on an EnVision® 2104 Multilabel Plate Reader (Perkin Elmer, Inc., Waltham, MA) using EnVision® Workstation version 1.13.3009.1401 software. Raw data was processed using the Envision® Workstation software. Responses were normalised to the wild-type variant response on the plate being analyzed, using the percentage of the response observed at the highest C1q concentration tested.

Normalised data were analysed in GraphPad Prism software v6.07 and data fitted using a 4-parameter logistic model. This was then used to calculate EC50s for full curves and curves approximating to full. A threshold for determining positivity was calculated as the mean response of Negative Fc variant (at maximum C1q concentration) plus 2× standard deviation, calculated separately for each plate. Binding potency was estimated by interpolation of the concentration at which signal exceeded the threshold (˜30% maximum) and the difference over wild-type was calculated using the equation [potency relative to WT=(x_{WT}/xₜₑₛₜ)×100], where x is the concentration of C1q at threshold.

The results are shown in Table 15.2. The relative binding of variants when compared within a given Fab combination was similar across the 3 antibody sets, with significant correlations between all the data sets. Binding of C1q to wild-type was observed at sub-nanomolar C1q concentrations, whereas the Fc negative variant (L234A, L235A, D265S) demonstrated little to no binding with a relative binding affinity more than 100-fold lower. Binding of C1q to the FcγRIIb-enhanced binding variants was variable. One subset of samples showed enhanced binding as compared to wild-type (variants v26370, v31188, v31213, v32212 and v32231), and a second subset showed little to no binding (control v12, v26774, v27092, v31191, v31192, v32242, v32274, v32292, v32293 and v32294), with the majority of the remaining variants demonstrating a small reduction in affinity for C1q.

TABLE 15.2
Relative C1q Binding Affinity of Variants Compared to Wild-Type

| | C1q Concentration at Onset | | | C1q - Variant Binding | | | EC₅₀ of C1q - Variant | | |
|---|---|---|---|---|---|---|---|---|---|
| | of Binding (nM) | | | Relative to WT (%) | | | Binding (nM) | | |
| Variant # | HER2 | CD19 | CD40 | HER2 | CD19 | CD40 | HER2 | CD19 | CD40 |
| WT | 0.16 | 0.18 | 0.41 | 100.00 | 100.00 | 100.00 | 0.88 | 0.31 | 1.49 |
| Neg | 24.67 | 29.55 | <LOD* | 0.37 | 0.61 | 0.00 | <LOD | <LOD | <LOD |
| V12 | 31.52 | 2.84 | <LOD | 0.47 | 6.72 | 0.00 | <LOD | <LOD | <LOD |
| SELF | 0.18 | 0.23 | 0.73 | 73.14 | 88.26 | 50.41 | 1.38 | 0.36 | 4.73 |
| 22096 | 0.10 | 0.23 | 0.54 | 133.33 | 88.26 | 68.14 | 0.54 | 0.37 | 2.19 |
| 26370 | 0.08 | 0.18 | 0.17 | 162.03 | 113.41 | 224.24 | 0.41 | 0.29 | 0.75 |
| 26774 | <LOD | 45.10 | 4.46 | 0.00 | 0.45 | 8.30 | <LOD | <LOD | <LOD |
| 27092 | <LOD | 20.32 | 23.06 | 0.00 | 0.94 | 1.71 | <LOD | <LOD | <LOD |
| 31186 | 0.46 | 0.35 | 2.47 | 27.77 | 58.00 | 15.01 | <LOD | 0.83 | <LOD |
| 31188 | 0.05 | 0.14 | 0.13 | 266.67 | 149.26 | 293.65 | 0.28 | 0.22 | 0.27 |
| 31191 | 2.69 | 1.92 | 11.26 | 4.75 | 10.58 | 3.29 | <LOD | <LOD | <LOD |
| 31192 | <LOD | 184.90 | <LOD | <LOD | 0.11 | <LOD | <LOD | <LOD | <LOD |
| 31213 | 0.06 | 0.16 | 0.15 | 220.00 | 85.09 | 256.21 | 0.30 | 0.23 | 0.37 |
| 32210 | 1.02 | 0.32 | 1.00 | 21.08 | 58.62 | 47.06 | <LOD | 0.64 | 2.80 |
| 32211 | 0.60 | n.d. | 1.27 | 35.83 | n.d. | 37.14 | n.d. | <LOD | 5.95 |
| 32212 | 0.06 | 0.15 | 0.22 | 227.59 | 94.48 | 175.78 | 0.31 | 0.24 | 0.48 |
| 32226 | 0.23 | 0.28 | 1.14 | 63.25 | 68.95 | 34.44 | <LOD | 0.58 | <LOD |
| 32227 | 0.61 | 0.28 | 1.51 | 35.13 | 66.08 | 31.18 | <LOD | 0.51 | <LOD |
| 32230 | 0.24 | 0.20 | 0.85 | 55.00 | 67.16 | 45.96 | 2.45 | 0.37 | 2.95 |
| 32231 | 0.06 | 0.14 | 0.18 | 238.71 | 133.57 | 223.86 | 0.30 | 0.23 | 0.39 |
| 32242 | 16.46 | 7.79 | 8.75 | 1.31 | 2.40 | 5.40 | <LOD | <LOD | <LOD |
| 32274 | <LOD | <LOD | 6.91 | <LOD | <LOD | 5.70 | <LOD | <LOD | <LOD |
| 32282 | 2.88 | 0.55 | 1.57 | 4.59 | 25.09 | 24.94 | <LOD | 2.14 | <LOD |
| 32284 | 6.20 | 0.93 | 1.58 | 2.13 | 14.68 | 24.87 | <LOD | <LOD | <LOD |
| 32287 | 0.94 | 0.35 | 1.30 | 14.06 | 38.81 | 30.13 | <LOD | 0.92 | <LOD |
| 32288 | 2.22 | 0.48 | 1.26 | 5.94 | 28.36 | 31.06 | <LOD | 1.63 | <LOD |
| 32292 | 39.17 | 132.81 | 24.71 | 0.55 | 0.14 | 1.91 | <LOD | <LOD | <LOD |
| 32293 | 32.04 | <LOD | 36.26 | 0.67 | <LOD | 1.30 | <LOD | <LOD | <LOD |
| 32294 | 14.65 | 8.26 | 23.89 | 1.47 | 2.26 | 1.98 | <LOD | <LOD | <LOD |
| 32295 | 26.41 | 88.39 | 1.34 | 0.81 | 0.21 | 35.33 | <LOD | <LOD | 4.48 |
| 32296 | 6.35 | <LOD | 1.43 | 2.08 | <LOD | 27.37 | <LOD | <LOD | <LOD |

*<LOD: signal response was below limit of detection for the assays

### In Vitro CDC Assay

The ability of the variants to activate the complement cascade and induced membrane attack complex-based lysis of cells was evaluated in an in vitro cell assay utilising Ramos cells opsonised with the anti-CD40 antibody variants. Ramos-(RA1) cells were seeded into 96-well assay plate wells at 1e5 cells/well in 50 μl RPMI buffer. Test antibodies and rituximab as control were prepared as 7-step 1:3 serial dilutions in RPMI buffer were added 1:2 to test wells and incubated at ambient temperature for 20 min. Human serum, either active or heat-inactivated by incubation at 560 for 30 min, was added 1:3 to test wells and incubated at 37° C., 500 CO₂ for 2.5 hours. Final assay conditions were 1e6 cells/ml, 25% human serum (v/v) and test antibody 7-point 4-fold dilution series starting at 10 μg/ml as the highest concentration. Following incubation, 100 L of CellTiter-Glo® (Thermo Fisher Scientific, Waltham, MA) was added to each test well and incubated at ambient temperature for 10 min with agitation. Plates were read on an EnVision® Plate Reader (Perkin Elmer, Inc., Waltham, MA) using a 700 nm luminescence filter and EnVision® Manager software.

Percent lysis was calculated for each condition as 100×(1−(test signal/mean of untreated control samples)). The maximum lysis observed for each test sample was defined as the mean percentage lysis observed at the highest antibody concentration tested and was normalised to wild-type. The percent lysis data was analysed in GraphPad Prism software v5.0.4 (GraphPad Software, San Diego, CA) and data fitted using a 4-parameter logistic model to generate a dose-response model. These models were then used to interpolate the concentration of antibody required to induce 20% lysis of sample, which was defined as the measure of the antibody potency. Variants were assayed in 3 independent experiments. In the third experimental run, the concentration of rituxumab required to reach the 20% lysis threshold was approximately 5-fold higher than the previous repeats. This was also observed for all the test variants except wild-type. For analysis, therefore, the potency was normalised to the rituximab control within run using the equation [potency relative to positive control=(x_{positive control}/xₜₑₛₜ)×100], where x is the concentration of antibody at threshold. The potency of wild-type from run 3 was excluded from the subsequent data analysis as an outlier. Potency values were then further normalised as a percentage of wild-type using the mean potency of the rituximab-normalised wild-type variants from runs 1 and 2 only.

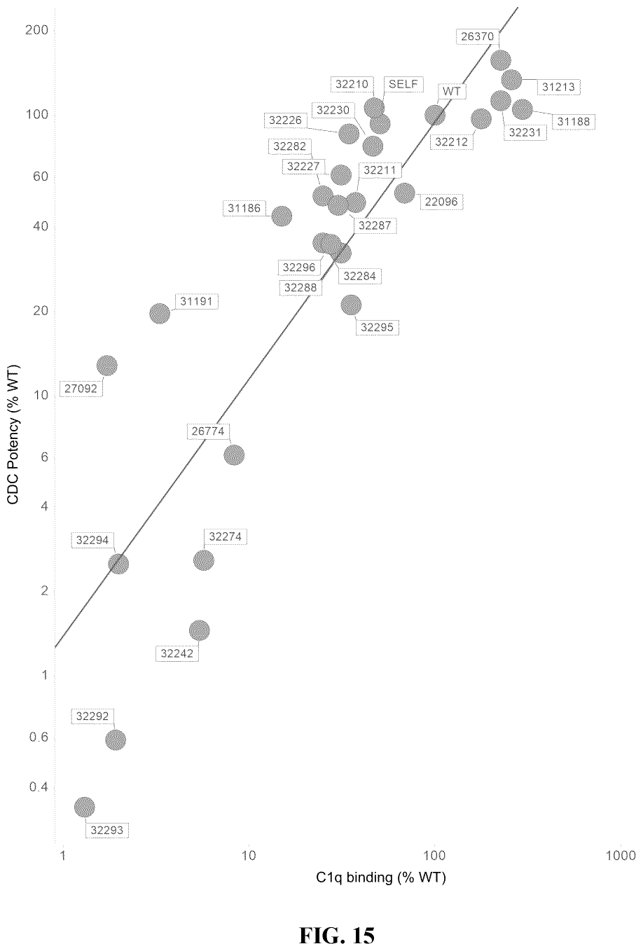

The results with active serum are shown in Table 15.3. No cell lysis was observed when antibody-treated cells were incubated in the presence of heat-inactivated serum, as expected. The rituximab positive control demonstrated a dose-dependent increase in cell lysis in all 3 experiments, with a maximal lysis of 96-99%. CDC activity was observed for wild-type with lysis above threshold observed at sub-nanomolar antibody concentrations, whereas the Fc negative variant control induced no measurable increase in lysis. A significant correlation between the C1q binding and CDC activity was observed (see FIG. 15) with variants v26370, v31213 and v32231 possessing greater potency than wild-type, whereas control v12 and variants v31192, v32242, v32292, v32293 and v32294 induced very little lysis even at the highest antibody concentration tested. Table 15.3: Potency of Variants in CDC Assay

TABLE 15.3
Potency of Variants in CDC Assay

| | Lysis (%)¹ | | Potency² (ng/mL) | | Normalized Potency³ | |
|---|---|---|---|---|---|---|
| Variant # | Mean | St Dev | Mean | St Dev | Mean | St Dev |
| 21653 | 77.82 | 8.33 | 67.65 | 19.47 | 100.00 | —⁴ |
| Neg | 5.13 | 7.29 | <LOD⁵ | <LOD | <LOD | <LOD |
| v12 | 7.28 | 4.76 | <LOD | <LOD | <LOD | <LOD |
| SELF | 78.19 | 11.17 | 165.50 | 114.17 | 93.56 | 37.83 |
| 31186 | 43.82 | 9.36 | 343.86 | 202.71 | 43.81 | 19.90 |
| 31188 | 78.06 | 9.32 | 154.38 | 137.62 | 104.86 | 26.77 |
| 31191 | 27.02 | 3.21 | 1906.35 | 1819.86 | 19.60 | 26.58 |
| 31192 | 0.64 | 8.35 | <LOD | <LOD | <LOD | <LOD |
| 26774 | 34.99 | 12.95 | 2378.55 | 1532.09 | 6.15 | 2.26 |
| 22096 | 66.25 | 0.42 | 290.34 | 242.99 | 52.69 | 8.27 |
| 26370 | 64.29 | 15.21 | 93.89 | 67.66 | 156.99 | 45.98 |
| 27092 | 27.85 | 2.11 | 1401.40 | 666.71 | 12.84 | 11.55 |
| 32274 | 27.13 | 21.61 | 2930.64 | —⁴ | 2.58 | —⁴ |
| 32282 | 45.48 | 7.64 | 379.87 | 237.89 | 51.71 | 43.77 |
| 32284 | 40.09 | 15.63 | 549.39 | 347.85 | 35.17 | 23.82 |
| 32296 | 36.63 | 22.14 | 477.40 | 271.64 | 34.67 | 19.33 |
| 32287 | 46.75 | 7.80 | 314.89 | 252.62 | 47.85 | 9.14 |
| 32288 | 41.69 | 5.79 | 521.81 | 370.06 | 32.31 | 15.18 |
| 32212 | 65.28 | 1.75 | 182.90 | 194.78 | 97.00 | 11.27 |
| 32230 | 44.91 | 2.94 | 194.43 | 140.30 | 77.49 | 24.30 |
| 31213 | 68.16 | 7.26 | 112.73 | 89.43 | 133.95 | 30.69 |
| 32231 | 66.24 | 9.02 | 157.63 | 164.44 | 112.87 | 26.70 |
| 32226 | 32.40 | 2.44 | 214.57 | 200.33 | 85.69 | 38.56 |
| 32227 | 46.58 | 10.22 | 292.99 | 299.01 | 61.08 | 19.17 |
| 32210 | 66.53 | 4.96 | 172.82 | 172.25 | 106.39 | 44.75 |
| 32211 | 36.37 | 1.99 | 308.18 | 237.07 | 48.89 | 12.11 |
| 32242 | 12.79 | 9.44 | 5211.53 | —⁴ | 1.45 | —⁴ |
| 32292 | 12.77 | 7.48 | 13375.12 | —⁶ | 0.59 | —⁶ |
| 32293 | 14.98 | 3.87 | 31371.62 | —⁶ | 0.34 | —⁶ |
| 32295 | 52.55 | 35.49 | 1172.30 | 1327.26 | 21.09 | 15.97 |
| 32294 | 17.70 | 10.92 | 3007.12 | —⁴ | 2.51 | —⁴ |
| Rituximab | 97.54 | 1.23 | 92.39 | 90.42 | 179.09 | —⁷ |

¹At highest test antibody concentration (10000 ng/mL)
²Antibody concentration above which >20% cell lysis was achieved
³Normalized to rituxumab then rescaled against WT
⁴20% lysis was not achieved with this sample in 1 of the 3 repeats
⁵<LOD: 20% lysis was not achieved with sample in any of the 3 repeats
⁶20% lysis was not achieved with this sample in 2 of the 3 repeats
⁷Sample used as normalization control (variation not relevant)

### Example 16: In Vitro Immunogenicity

The introduction of mutations and loop sequences into the variants has the potential to increase the risk that they may induce an immune response. Clinical immunogenicity assessments typically detect and characterize anti-drug antibodies (ADA) which are predominantly CD4 T cell dependent. Hence, activation and proliferation of CD4 T cells is generally required for induction and is used as a marker for potential immunogenicity risk (Koren, et al., 2008, J. Immunol Methods, 333(1-2):1-9; Shankar, et al., 2007, Nat Biotechnol, 25(5):555-561). The immunogenicity of the variants from Example 14 in combination with anti-HER2 Fabs was, therefore, evaluated in an in vitro whole PBMC (peripheral blood mononuclear cells) proliferation assay.

### Method

PBMC samples with known HLA haplotypes were purchased from BioIVT Inc (Westbury, NY). A panel of ten (first experiment) or twelve (second experiment) individual donors expressing HLA class II DRB1 alleles representative of a diverse population was selected.

PBMC were labelled with carboxyfluorescein succinimidyl ester (CFSE) (Invitrogen Corporation, Carlsbad, CA; C34554) by incubation of cells at 5e6 cells/ml in RPMI media supplemented with 5% human AB serum (Sigma-Aldrich, St. Louis, MO; H3677) and 250 nM CFSE for 10 min. Cells were then pelleted by centrifugation at 400 rcf at ambient temperature for 5 min then re-suspended in RPMI media supplemented with 5% human AB serum and seeded at 4e6 cells/well in a 24-well culture plate. Test samples were added to cells to a final concentration of 50 μg/ml. Tuberculin Purified Protein Derivative (PPD, Statens Serum Institute, Batch RT51, lot #235) was added to cells to a final concentration of 2 μg/ml as a positive control. Test samples and positive control were assayed in triplicate. Six replicates of untreated cells were included as a baseline control. Cells were cultured at 37° C. and 5% CO₂ for 72h. Cells were re-challenged by removal of half the culture media and addition of fresh RPMI media supplemented with 5% human AB serum, test sample at test concentration as above and 2.5 ng/mL rhIL2 (R&D Systems, Minneapolis, MN; 202IL) then incubated as before for 96h. Cells were pelleted by centrifugation as above then re-suspended in 100 μl of a 1:1000 dilution of viability stain (BV510, BD Biosciences, San Jose, CA; #564406) in PBS and incubated at ambient temperature for 15 min. Cells were pelleted by centrifugation as before then resuspended in 100 μl of 1:12 anti-CD3/APC (BD Bioscience, #340440) and 1:12 anti-CD4/PerCPcy5.5 (BD Biosciences, #560650) antibodies in MACS rinsing solution (Miltenyi Biotech, Bergisch Gladbach, Germany; #130-91-222) supplemented with 0.5% (v/v) BSA (Miltenyi, #130-091-222) and incubated at ambient temperature for 20 min. Cells were then pelleted by centrifugation as above and resuspended in 250 μl of MACS rinsing solution with 0.5% BSA. CD4 T lymphocyte proliferation was then measured by CSFE dilution by flow cytometry using a FACSCanto™ 10 flow cytometer (BD Biosciences, San Jose, CA) with CFSE detected using 488 nm excitation and 530/30 nm bandpass filter, APC detected using 640 nm excitation and 670/30 nm bandpass filer and PerCPcy5.5 detected using 488 nm excitation and 595/40 nm bandpass filter.

Proliferating T lymphocytes were defined as CFSE^{dim}, CD3⁺ CD4⁺. Data was analysed using FlowJo™ FACS software (Becton, Dickinson and Company, Franklin Lakes, NJ) and events gated for live cells (BV540 negative), lymphocytes (SSC-A v FSC-A), single cells (FSC-H v FSC-A), CD4⁺ T lymphocytes (CD3⁺ and CD4⁺) and proliferated cells (CFSE^{dim}). The counts of proliferated CD4+T lymphocytes were reported as a proportion of the total CD4⁺ population for each sample. A mixed-effects model of medium-treated responses with Plate, Donor, and their interaction as random effects was used to compute observations' studentized residuals. An analytical outlier was any observation with a studentized residual less than −3 or greater than 3. These observations were removed from the remainder of the analysis. The data from untreated cells was analysed to identify outliers and used to establish baseline proliferation of each donor by calculating the mean signal of samples. A fixed-effects model was applied to all data excluding outliers—with Treatment, Donor, Plate, and all two- and three-way interactions as fixed effects; and with residual variance estimates that varied by Treatment. This enabled statistical contrasts of each test article's mean response to the plate-specific medium-treated mean response for each donor to be calculated.

The stimulation index (SI) of a treatment was defined as the ratio of the geometric mean of percentage proliferated cells against that of the untreated cells. For each donor, the difference to medium of log 10-transformed response (equivalent to log 10-transformed SI) was evaluated for clinical significance (if the value of the contrast difference exceeded the previously-established assay response threshold of 1.71 SI) and statistical significance (unadjusted p-value from the two-sided contrast test was less than the significance level of 0.05). Any donor whose response met both criteria was considered a positive response for a given test article. Percent Immunogenicity was calculated as the proportion of positive responses out of total donors. Strength of response was calculated as the mean SI across positive responding donors. A response index (RI) for each test article was calculated using the following equation: RI=Proportional Immunogenicity (frequency of response)×mean SI across positive responders (strength of response).

### Results

The percentage of CD4+ T cells undergoing proliferation was measured for all test molecules and the PPD positive control. All samples were tested in triplicate for each PBMC donor, with 6 replicates of the medium only negative control included for baseline comparison. The proportional proliferation relative to medium only was calculated for each donor and defined as the stimulation index (SI). A statistical difference between test medium (P<0.05) and an average response of greater than or equal to 1.71 was deemed a meaningful response. The response index (RI) was defined as the mean SI of the positive donor responses multiplied by the proportion of positive donors and was considered as a measure of the strength of response.

The results are shown in Table 16.1. The PPD positive control showed 100% positive responses in all the test donors in both experiments. In the first experiment positive responses were produced by all 5 of the antibodies tested, with wild-type generating a single positive result, one variant 2 positive results and the remaining variants 3 positive results amongst the tested donors. The mean SI of the positive responses for each sample tested ranged from 1.96 to 3.45 as compared to 55.5 for the positive control. In the second experiment, 4 of the tested antibodies, including wild-type, produced no positive responses, 4 produced a single positive response and 2 antibodies produced 2 positive responses. The mean SI of the positive responses of each sample ranged from 1.75 to 7.41 as compared to 50.6 for the positive control. Three of the samples (wild-type and variants v31187 and v31274) tested in the first experiment were re-tested in the second. These samples produced 1, 2 and 3 positive results, respectively, out of 10 in the first experiment. However, there were no positive responses out of 12 for these samples in the second experiment. Two donors (BRH1367704 and BRH11367709) were present in both experimental sets: in the first experiment significant responses were observed for the wild-type and variant 1274 with BRH1367709 and for variants v31274 and v31187 with BRH1367704, but no significant responses were observed in the second experiment.

Overall, the results indicate that the positive responses observed for the variants in the first experiment are marginal and that the immunogenicity risk of the Fc modifications is low.

TABLE 16.1

| | | | | | | |
|---|---|---|---|---|---|---|
| | | Donors | | Mean SI | Mean SI | |
| | Total | with | Proportion | Response | Response | Response |
| | Donors | Positive | of Positive | (of all | (of Positive | Index |
| Variant # | Tested | Response | Donors | Donors) | Donors) | (RI) |

Evaluation of 5 anti-HER2 Fc variants across 10 donors

| | | | | | | |
|---|---|---|---|---|---|---|
| PPD | 10 | 10 | 100 | 55.46 | 55.46 | 55.46 |
| 31274 | 10 | 3 | 33 | 1.34 | 1.96 | 0.65 |
| 31191 | 10 | 3 | 33 | 1.38 | 2.39 | 0.79 |
| 31256 | 10 | 3 | 33 | 1.36 | 2.20 | 0.73 |
| 31187 | 10 | 2 | 20 | 1.41 | 2.86 | 0.57 |
| 21653 | 10 | 1 | 10 | 1.16 | 3.45 | 0.35 |

Evaluation of 9 anti-HER2 Fc variants across 12 donors

| | | | | | | |
|---|---|---|---|---|---|---|
| PPD | 12 | 12 | 100 | 50.62 | 50.65 | 50.65 |
| 21653 | 12 | 0 | 0 | 1.03 | NA | 0 |
| 31274 | 12 | 0 | 0 | 1.22 | NA | 0 |
| 32226 | 12 | 1 | 8 | 1.23 | 2.17 | 0.17 |
| 32227 | 12 | 1 | 8 | 1.12 | 1.75 | 0.14 |
| 32274 | 12 | 2 | 17 | 1.24 | 2.00 | 0.34 |
| 31187 | 12 | 0 | 0 | 0.70 | NA | 0 |
| 31188 | 12 | 0 | 0 | 0.69 | NA | 0 |
| 32284 | 12 | 1 | 8 | 0.81 | 2.19 | 0.18 |
| 32295 | 12 | 1 | 8 | 0.78 | 7.41 | 0.59 |

### Example 17: In Vivo Evaluation

Previous studies have demonstrated that antibody interaction with FcγR2b is the primary mechanism for uptake of immune complexes in vivo and that target antigen clearance can be enhanced by increasing affinity for the receptor (Iwayanagi, et al., 2015, J Immunol, 195(7):3198-3205). To explore the functional impact of the variants, the clearance of a soluble test antigen (human C5) was evaluated in transgenic human FcγRIIb mice using a steady-state model in which soluble antigen was delivered using an osmotic pump. Variants with improved FcγRIIb affinity and selectivity were tested in combination with anti-C5 Fabs having pH-selective affinity for human C5 (approximately 30 pM KD at pH7.4 as compared to approximately 500 pM at pH6.0).

### Methods

Animals: C57BL/6J mice (wild-type mice) were purchased from Charles River Laboratories (Wilmington, MA) and hFcγRIIb transgenic (Tg) mice on a C57BL/6J background (strain B6.FVB-Tg(hFcγRIIB)/J) were licensed from Mark Cragg (University of Southampton, U.K., see Roghanian, et al., 2015, Cancer Cell, 27:473-488). Individual mice were evaluated for human FcγRIIb expression prior to study initiation by flow cytometery analysis of mouse primary B cells and monocytes from blood. To 30 μl of mouse blood was added 1 μl of Trustain FcX (BioLegend, San Diego, CA; 11320) to block murine Fc and samples were incubated for 5 min at 4° C. To samples was then added either rat anti-mouse CD19 antibody conjugated to APC (MACS, 130-102-546), hamster anti-mouse CD80 antibody conjugated to BV 421 (Beckton, Dickinson and Company, Franklin Lakes, NJ; 562611) or rat anti-mouse CD11b antibody conjugated to BV 421 (BioLegend 101236) in combination with mouse anti-human CD32 antibody conjugated to FITC (Becton Dickinson, 555448). Plates were incubated on ice for 30 min then 200 μl of 1× FACS lyse (Becton Dickinson, 349202) was added per sample. Samples were incubated at ambient temperature for 10 min then cells pelleted by centrifugation at 200×g for 5 min. Cells were washed twice with PBS supplemented with 1% (w/v) BSA and 0.1% (w/v) sodium azide then resuspended in PBS and analysed using a CytoFLEX flow cytometer (Beckton, Dickinson and Company, Franklin Lakes, NJ). Data was analysed using FlowJo™ FACS software version 7.6.5 and the events gated for lymphocytes and monocytes (SSC-A/FSC-A), doublet exclusion (FSC-H/FSC-A) and B lymphocytes, monocytes or activated monocytes by positive staining for CD19, CD11b or CD80, respectively. The proportion of each cell population staining positive for human CD32 was then calculated for each cell type of each sample. Allocation of mice to treatment groups was randomised using sex as a blocking factor and human FcγRIIb expression level as a covariate using software R version 3.5.0.

Immunohistochemical Analysis for FcγRIIb Expression: Tissue samples were fixed in 4% paraformaldehyde for 24h, processed using a Tissue Tek VIP® (Sakura Finetech USA, Inc., Torrance, CA) then embedded in paraffin. Paraffin blocks were sectioned to show the full tissue surface and samples stained with hematoxylin and eosin for general structural observations. Samples were pre-treated with cell conditioning solution 2 (Roche Diagnostics, Basel, Switzerland; 0542454200). Human FcγRIIb was detected by incubation with a goat anti-human CD32B antibody (Abcam, Inc., Cambridge, MA; Ab77093) at 3.8 μg/ml for 1 h followed by rabbit anti-goat secondary (Thermo Fisher Scientific, Waltham, MA; A27011) at 2 μg/ml after which sections were developed using anti-rabbit HW, anti-HQ HRP and DAB stain on a Ventana BenchMark ULTRA (Roche Diagnostics). Samples were then chemically dehydrated and a cover-slip added prior to imaging.

In vivo Study of Antibody Pharmacokinetics (single dose in hFcγRIIb Tg mice): Antibodies against human C5 with differing affinities for human FcγRIIb were administered intravenously at 1 mg/kg to mice using 5 mice per dose group. Blood samples (1×10 μl) were taken from animals pre-dose, 0.25, 3, 6, 24, 48, 72, 96, 120, 168, 336 and 504h post dose via tail vein bleed collected into an EDTA capillary tube. Each aliquot of collected blood was then transferred into a micronic tube containing an equal volume of water, gently mixed and stored frozen at −20° C. One animal from each group was euthanised and liver and spleen removed and fixed for histology at 24, 120 and 504h post dose.

In vivo Study of Antibody Pharmacokinetics and Target Clearance (single dose): An infusion pump (Alzet) filled with 1000 μg/ml human C5 (hC5, Complement Technology, Inc., Tyler, TX; A120) was implanted under the skin on the back of wild-type or hFcγRIIb Tg C57BL/6 mice to prepare a mouse model with a constant plasma concentration of hC5. Approximately 1 hr before implantation, mice were given a 0.5 mg/kg loading dose of 0.1 mg/ml human C5 in order to bring circulating levels of hC5 close to that of the steady state at the point of pump implantation. Antibodies against human C5 with differing affinities for human FcγRIIb were administered intravenously at 1 mg/kg to mice with 5 mice per dose group. Blood samples (2×10 μl) were taken from animals pre-dose, 0.25, 3, 6, 24, 48, 96 and 120h post dose via tail vein bleed collected into an EDTA capillary tube. Each aliquot of collected blood was then aliquoted into a micronic tube containing an equal volume of water, gently mixed and stored frozen at −20° C.

Evaluation of Antibody and hC5 Concentrations: Plasma anti-human C5 antibody levels were determined from collected blood samples by immunoassay using a GyroLab® xPand (Gyros Protein Technologies, Uppsala, Sweden). Antibody standard curves were prepared in Rexxip A buffer (Gyros Protein Technologies, Uppsala, Sweden) as 8-point curves from 30,000 ng/ml to 10 ng/ml. Test antibodies were captured using a goat anti-human IgG F(Ab′)2 (Jackson Laboratory, Bar Harbor, ME; #109-006-097) biotinylated using a 10-fold molar excess of Sulfo-NHS-LC-Biotin (Thermo Fisher Scientific, Waltham, MA; #21327). Captured antibodies were detected using a goat anti-human kappa light chain antibody (BioRad Laboratories, Hercules, CA; STAR164) labeled with Alexa647 using a commercial labelling kit (Invitrogen Corporation, Carlsbad, CA; #A20186).

Plasma human C5 concentrations were determined by ELISA using a commercial anti-human C5 ELISA kit (Abcam, ab125963) with a standard curve prepared using human C5 (Complement Technology, A120). C5 ELISA plates were evaluated by absorbance at 450 nm using a SpectraMax® M5e plate reader (Molecular Devices, Wokingham, U.K.). The standard curve was plotted as a variable slope (four parameters), non-linear regression curve fit and the unknown values extrapolated accordingly using GraphPad Prism software version 5.0.4.

Data Analysis: Pharmacokinetic analysis was performed by non-compartmental pharmacokinetic analysis using WinNonlin™ (WNL), Version 8.1 (Certara, Princeton, NJ). All computations utilised the nominal sampling times. The systemic exposure was determined by calculating the area under the serum concentration time curve (AUC) from the start of dosing to the last quantifiable time point (AUCO-t) using the linear log trapezoidal method. The maximum observed peak serum concentration (Cmax) and the time at which it was observed (Tmax) were determined by inspection of the observed data. In addition, where applicable the total serum clearance (CL), volume of distribution at steady-state (Vss), terminal half-life (t½) and mean residence time (MRT) were calculated.

Statistical analysis was conducted on both pharmacodynamic and pharmacokinetic data sets to determine the difference between treatment groups. Pharmacodynamic data was analysed as a repeated measures ANOVA evaluating differences between groups and accounting for animal gender, baseline weight and differences in FcγRIIb expression. Pharmacokinetic data was analysed using an ANOVA to evaluate the differences between the Area Under the Curve variable only. An analysis of variance was also used to determine differences between animal sex and FcγRIIb expression.

### Results

Histologic comparison of human FcγRIIb expression in human, non-transgenic mouse and transgenic hFcγRIIb mouse livers revealed staining for human FcγRIIb in the human and transgenic mouse samples but not non-transgenic mouse samples. Staining was localised to hepatic lobules, consistent with expression in sinusoidal epithelial cells as previously reported (Ganesan, et al., 2012, J Immunol, 189(10):4981-4988). Expression levels of FcγRIIb in individual mice were confirmed by flow cytometry analysis prior to study.

Transgenic mice at a steady serum concentration of human C5 were dosed 1 mg/kg with five anti-C5 antibody Fc variants: anti-C5 with wild-type human IgG1 CH2 domain (WT); anti-C5 with abrogated binding to FcγRIIb (Neg); and three variants (v31188, v32227 and v32284; see Example 14) with differing degrees of enhanced affinity and selectivity for human FcγRIIb.

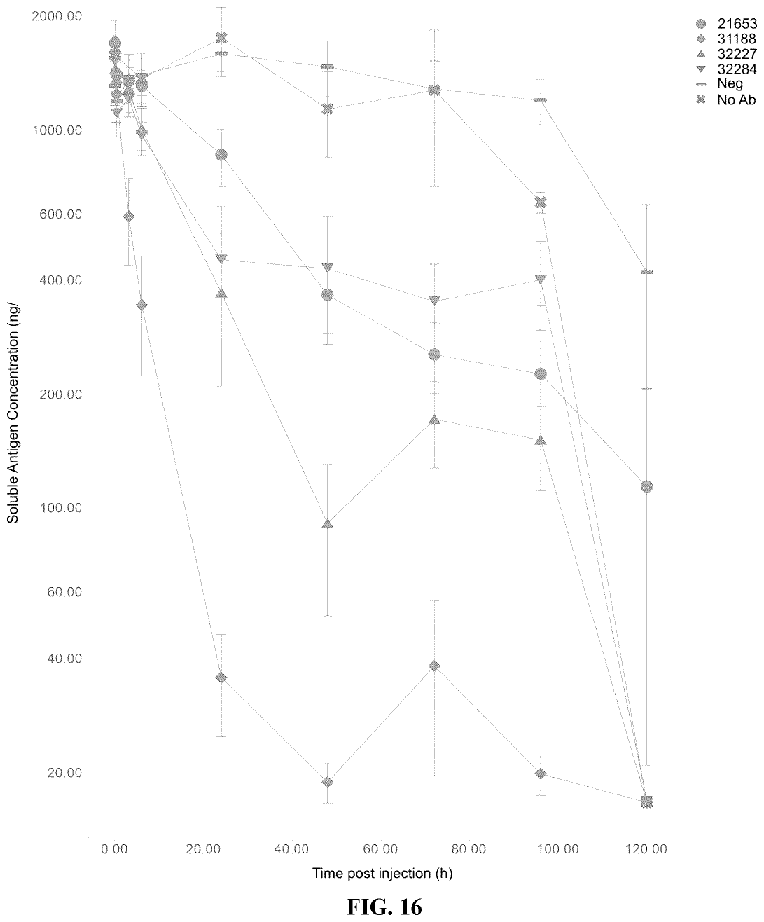

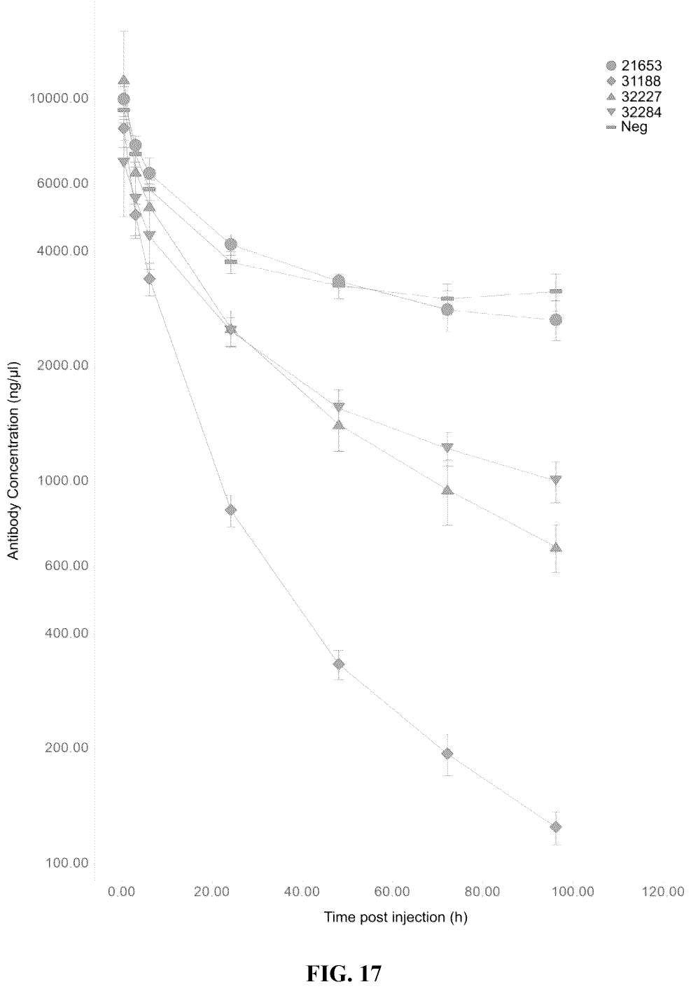

The results are shown in FIGS. 16 and 17. As can be seen from FIG. 16, the rate of clearance of soluble antigen varied in a FcγRIIb affinity-dependent manner with faster clearance of antigen observed with increasing affinity for FcγRIIb. In the absence of antibody, antigen levels remained at a steady-state serum concentration for approximately 96 h before rapidly reducing to below detectable levels by 120 h. Addition of control (Neg) antibody with abrogated binding to FcγR (but not FcRn) did not cause a reduction in circulating antigen level and may even have stabilised circulating levels as evidenced by the higher levels observed at later timepoints as compared to the no antibody control. In contrast, levels of circulating antigen were reduced for all antibodies possessing affinity for human FcγRIIb as compared to the abrogated variant. The variant v31188 which possesses the strongest affinity for FcγRIIb resulted in the fastest antigen clearance of all antibodies tested and this was significantly different from the WT and variant v32284. Variant v32227 was also significantly different from WT. Initial clearance of antigen by variant v32284 appeared similar to that of variant v32227 but appeared to plateau after 24h at a higher steady-state level.

The concentration of dosed antibody over time also varied in a FcγRIIb affinity-dependent manner with serum concentration reducing more quickly with increasing affinity for human FcγRIIb (FIG. 17). Serum antibody levels of WT and the FcγRIIb-abrogated control were not significantly different, whereas all tested variants with enhanced binding to human FcγRIIb were cleared significantly faster than WT.

Antibody variants were also dosed at 1 mg/kg into mice which did not receive any soluble target antigen. The observed pharmacokinetics of each variant was comparable to that measured in the presence of antigen, indicating that binding to antigen did not impact the clearance of variants from circulation.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

### Tables

Table 6.17 presents the results for all variants generated by Strategy 1 as described in Example 6. “Control” for IIb and IIaR binding and IIb selectivity is variant v27293.

Table 6.18 presents the results for all variants generated by Strategy 2 as described in Example 6. “Control” for IIb and IIaR binding and IIb selectivity is variant v27294.

Table 6.19 presents the results for all variants generated by Strategy 3 as described in Example 6. “Control” for IIb and IIaR binding and IIb selectivity is variant v27362.

Table 6.20 presents the results for all variants generated by Strategy 4 as described in Example 6. “Control” for IIb and IIaR binding and IIb selectivity is variant v27362.

Table 6.21 presents the results for all variants generated by Strategy 5 as described in Example 6. “Control” for IIb and IIaR binding and IIb selectivity is variant v27293.

Table 6.22 lists Strategy 1 variants that meet Criteria A for FcγRIIb selectivity and affinity as described in Example 6. “Control” for IIb and IIaR binding and IIb selectivity is variant v27293.

Table 6.23 lists Strategy 2 variants that meet Criteria A for FcγRIIb selectivity and affinity as described in Example 6. “Control” for IIb and IIaR binding and IIb selectivity is variant v27294.

Table 6.24 lists Strategy 3 variants that meet Criteria A for FcγRIIb selectivity and affinity as described in Example 6. “Control” for IIb and IIaR binding and IIb selectivity is variant v27362.

Table 6.25 lists Strategy 1 variants that meet Criteria B for FcγRIIb selectivity and affinity as described in Example 6. “Control” for IIb and IIaR binding and IIb selectivity is variant v27293.

Table 6.26 lists Strategy 2 variants that meet Criteria B for FcγRIIb selectivity and affinity as described in Example 6. “Control” for IIb and IIaR binding and IIb selectivity is variant v27294.

Table 6.27 lists Strategy 3 variants that meet Criteria B for FcγRIIb selectivity and affinity as described in Example 6. “Control” for IIb and IIaR binding and IIb selectivity is variant v27362.

TABLE 6.17
Strategy 1 Variants

| | | | | | | IIb- | | IIaR- | | IIb | |
|---|---|---|---|---|---|---|---|---|---|---|---|
| | | | | | | Fold | | Fold | | Selectivity² | |
| | Variant | | FcγRIIb | FcγRIIaR | IIb- | wrt | IIaR- | wrt | IIb | Fold wrt | |
| Strategy | # | Mutations¹ | K_{D} | K_{D} | Fold | Control | Fold | Control | Selectivity² | Control | ELISA³ |
| Controls | 16463 | WT | 1.4E−06 | 3.1E−07 | 1.0 | | 1.0 | | 1.0 | | |
| | 27293 | strat1_control | 3.5E−09 | 8.8E−09 | 415.4 | 1.0 | 35.2 | 1.0 | 11.8 | 1.0 | 70.76 |
| | | (A_G236N_G237A | | | | | | | | | |
| | | B_G236D_G237F_ | | | | | | | | | |
| | | S239D_S267V_H268D_ | | | | | | | | | |
| | | Template_1) | | | | | | | | | |
| | 28472 | strat1_control + E269K | 5.0E−09 | 1.8E−08 | 290.7 | 0.7 | 17.0 | 0.5 | 17.1 | 1.4 | 59.75 |
| | v12⁴ | Symmetrical | 1.3E−08 | 2.2E−07 | 111.6 | | 1.4 | | 80.5 | | 100.12 |
| | | E233D_G237D_P238D_ | | | | | | | | | |
| | | H268D_P271G_ | | | | | | | | | |
| | | A330R | | | | | | | | | |
| Strat1- | 26093 | A_L234G_strat1 | 3.4E−09 | 9.7E−09 | 424.3 | 1.0 | 31.7 | 0.9 | 13.4 | 1.1 | 37.91 |
| Chain A | 26094 | A_L234A_strat1 | 4.7E−09 | 1.4E−08 | 306.8 | 0.7 | 22.8 | 0.6 | 13.4 | 1.1 | 46.09 |
| | 26095 | A_L234V_strat1 | 4.3E−09 | 1.5E−08 | 334.7 | 0.8 | 20.8 | 0.6 | 16.1 | 1.4 | 46.93 |
| | 26096 | A_L234I_strat1 | 4.0E−09 | 1.4E−08 | 362.2 | 0.9 | 21.7 | 0.6 | 16.7 | 1.4 | 55.23 |
| | 26097 | A_L234M_strat1 | 3.8E−09 | 1.3E−08 | 378.8 | 0.9 | 23.4 | 0.7 | 16.2 | 1.4 | 44.52 |
| | 26098 | A_L234F_strat1 | 2.6E−09 | 1.1E−08 | 547.0 | 1.3 | 28.1 | 0.8 | 19.5 | 1.6 | 44.42 |
| | 26099 | A_L234W_strat1 | 1.3E−09 | 5.8E−09 | 1131.7 | 2.7 | 53.3 | 1.5 | 21.2 | 1.8 | 43.95 |
| | 26100 | A_L234Y_strat1 | 3.1E−09 | 1.0E−08 | 469.5 | 1.1 | 29.4 | 0.8 | 16.0 | 1.4 | 45.27 |
| | 26101 | A_L234T_strat1 | 3.3E−09 | 1.3E−08 | 442.9 | 1.1 | 24.1 | 0.7 | 18.4 | 1.6 | 38.40 |
| | 26102 | A_L234S_strat1 | 3.9E−09 | 1.4E−08 | 372.3 | 0.9 | 21.9 | 0.6 | 17.0 | 1.4 | 45.84 |
| | 26103 | A_L234Q_strat1 | 3.7E−09 | 1.5E−08 | 395.7 | 1.0 | 20.1 | 0.6 | 19.7 | 1.7 | 42.13 |
| | 26104 | A_L234N_strat1 | 3.1E−09 | 1.2E−08 | 460.2 | 1.1 | 26.7 | 0.8 | 17.2 | 1.5 | 54.00 |
| | 26105 | A_L234D_strat1 | 2.3E−09 | 1.1E−08 | 636.1 | 1.5 | 28.8 | 0.8 | 22.1 | 1.9 | 60.63 |
| | 26106 | A_L234E_strat1 | 6.3E−09 | 1.5E−08 | 229.2 | 0.6 | 20.0 | 0.6 | 11.5 | 1.0 | 52.77 |
| | 26107 | A_L234R_strat1 | 8.4E−09 | 2.5E−08 | 171.0 | 0.4 | 12.5 | 0.4 | 13.6 | 1.2 | 46.97 |
| | 26108 | A_L234K_strat1 | 8.2E−09 | 2.7E−08 | 177.1 | 0.4 | 11.5 | 0.3 | 15.4 | 1.3 | 49.22 |
| | 26109 | A_L234H_strat1 | 3.9E−09 | 1.3E−08 | 372.5 | 0.9 | 23.1 | 0.7 | 16.2 | 1.4 | 56.07 |
| | 26110 | A_L234P_strat1 | 4.2E−09 | 1.5E−08 | 346.0 | 0.8 | 20.5 | 0.6 | 16.9 | 1.4 | 53.74 |
| | 26111 | A_L235G_strat1 | 9.6E−09 | 2.6E−08 | 149.8 | 0.4 | 11.7 | 0.3 | 12.8 | 1.1 | 56.98 |
| | 26112 | A_L235A_strat1 | 5.1E−09 | 2.0E−08 | 284.5 | 0.7 | 15.3 | 0.4 | 18.7 | 1.6 | 56.24 |
| | 26113 | A_L235V_strat1 | 4.3E−09 | 1.6E−08 | 332.6 | 0.8 | 19.0 | 0.5 | 17.5 | 1.5 | 48.60 |
| | 26114 | A_L235I_strat1 | 4.2E−09 | 1.5E−08 | 347.7 | 0.8 | 20.1 | 0.6 | 17.3 | 1.5 | 55.29 |
| | 26115 | A_L235M_strat1 | 3.5E−09 | 1.4E−08 | 417.7 | 1.0 | 22.7 | 0.6 | 18.4 | 1.6 | 46.75 |
| | 26116 | A_L235F_strat1 | 1.1E−09 | 5.8E−09 | 1323.4 | 3.2 | 53.4 | 1.5 | 24.8 | 2.1 | 49.21 |
| | 26117 | A_L235W_strat1 | 8.6E−10 | 4.4E−09 | 1690.1 | 4.1 | 69.8 | 2.0 | 24.2 | 2.1 | |
| | 26118 | A_L235Y_strat1 | 1.2E−09 | 5.8E−09 | 1167.3 | 2.8 | 53.4 | 1.5 | 21.9 | 1.9 | 46.39 |
| | 26119 | A_L235T_strat1 | 5.2E−09 | 1.8E−08 | 279.7 | 0.7 | 16.9 | 0.5 | 16.6 | 1.4 | 46.00 |
| | 26120 | A_L235S_strat1 | 5.9E−09 | 1.6E−08 | 244.8 | 0.6 | 19.3 | 0.5 | 12.7 | 1.1 | 47.80 |
| | 26121 | A_L235Q_strat1 | 5.6E−09 | 2.0E−08 | 258.2 | 0.6 | 15.1 | 0.4 | 17.1 | 1.4 | 42.02 |
| | 26122 | A_L235N_strat1 | 6.3E−09 | 2.3E−08 | 229.2 | 0.6 | 13.4 | 0.4 | 17.1 | 1.4 | 43.26 |
| | 26123 | A_L235D_strat1 | 3.2E−09 | 1.3E−08 | 457.2 | 1.1 | 23.1 | 0.7 | 19.8 | 1.7 | 58.93 |
| | 26124 | A_L235E_strat1 | 3.1E−09 | 1.2E−08 | 466.3 | 1.1 | 25.0 | 0.7 | 18.7 | 1.6 | 57.16 |
| | 26125 | A_L235R_strat1 | 9.7E−09 | 3.9E−08 | 149.0 | 0.4 | 7.9 | 0.2 | 18.9 | 1.6 | 59.39 |
| | 26126 | A_L235K_strat1 | 1.2E−08 | 4.6E−08 | 121.5 | 0.3 | 6.8 | 0.2 | 18.0 | 1.5 | 61.67 |
| | 26127 | A_L235H_strat1 | 5.4E−09 | 2.1E−08 | 269.8 | 0.6 | 15.0 | 0.4 | 18.0 | 1.5 | 56.72 |
| | 26128 | A_L235P_strat1 | 6.5E−09 | 1.8E−08 | 223.6 | 0.5 | 17.1 | 0.5 | 13.1 | 1.1 | 51.74 |
| | 26129 | A_N236G_strat1 | 4.6E−09 | 8.9E−09 | 315.1 | 0.8 | 34.7 | 1.0 | 9.1 | 0.8 | 22.84 |
| | 26130 | A_N236A_strat1 | 4.0E−09 | 1.2E−08 | 357.6 | 0.9 | 26.8 | 0.8 | 13.4 | 1.1 | 36.37 |
| | 26131 | A_N236V_strat1 | 3.0E−09 | 8.0E−09 | 485.9 | 1.2 | 38.7 | 1.1 | 12.6 | 1.1 | 38.27 |
| | 26132 | A_N236L_strat1 | 2.7E−09 | 7.3E−09 | 539.0 | 1.3 | 42.2 | 1.2 | 12.8 | 1.1 | 34.92 |
| | 26133 | A_N236I_strat1 | 2.5E−09 | 8.3E−09 | 584.6 | 1.4 | 37.3 | 1.1 | 15.7 | 1.3 | |
| | 26134 | A_N236M_strat1 | 3.3E−09 | 1.2E−08 | 433.5 | 1.0 | 25.5 | 0.7 | 17.0 | 1.4 | 33.28 |
| | 26135 | A_N236F_strat1 | 2.4E−09 | 8.2E−09 | 602.9 | 1.5 | 37.4 | 1.1 | 16.1 | 1.4 | 41.70 |
| | 26136 | A_N236W_strat1 | 1.9E−09 | 6.8E−09 | 779.5 | 1.9 | 45.1 | 1.3 | 17.3 | 1.5 | |
| | 26137 | A_N236Y_strat1 | 1.6E−09 | 5.6E−09 | 882.8 | 2.1 | 55.0 | 1.6 | 16.0 | 1.4 | 40.84 |
| | 26138 | A_N236T_strat1 | 3.7E−09 | 1.2E−08 | 386.8 | 0.9 | 24.9 | 0.7 | 15.6 | 1.3 | 36.63 |
| | 26139 | A_N236S_strat1 | 3.4E−09 | 9.9E−09 | 425.3 | 1.0 | 31.3 | 0.9 | 13.6 | 1.2 | 38.48 |
| | 26140 | A_N236Q_strat1 | 3.9E−09 | 1.2E−08 | 373.0 | 0.9 | 25.6 | 0.7 | 14.6 | 1.2 | 41.40 |
| | 26141 | A_N236D_strat1 | 2.5E−09 | 8.4E−09 | 569.2 | 1.4 | 36.7 | 1.0 | 15.5 | 1.3 | 45.97 |
| | 26142 | A_N236E_strat1 | 3.6E−09 | 1.0E−08 | 405.0 | 1.0 | 30.5 | 0.9 | 13.3 | 1.1 | 36.19 |
| | 26143 | A_N236R_strat1 | 9.1E−09 | 3.1E−08 | 158.7 | 0.4 | 9.8 | 0.3 | 16.2 | 1.4 | 56.78 |
| | 26144 | A_N236K_strat1 | 1.2E−08 | 1.7E−08 | 118.2 | 0.3 | 18.4 | 0.5 | 6.4 | 0.5 | 54.72 |
| | 26145 | A_N236H_strat1 | 5.4E−09 | 1.6E−08 | 266.3 | 0.6 | 19.2 | 0.5 | 13.9 | 1.2 | 47.75 |
| | 26146 | A_N236P_strat1 | 6.1E−09 | 1.9E−08 | 235.4 | 0.6 | 16.4 | 0.5 | 14.3 | 1.2 | 54.62 |
| | 26147 | A_A237G_strat1 | 4.3E−10 | 1.6E−09 | 3394.8 | 8.2 | 194.0 | 5.5 | 17.5 | 1.5 | 50.63 |
| | 26148 | A_A237V_strat1 | 5.9E−09 | 9.9E−09 | 245.4 | 0.6 | 31.1 | 0.9 | 7.9 | 0.7 | 14.05 |
| | 26149 | A_A237L_strat1 | 5.7E−09 | 2.5E−08 | 251.9 | 0.6 | 12.5 | 0.4 | 20.1 | 1.7 | 59.50 |
| | 26150 | A_A237I_strat1 | 4.1E−09 | 5.1E−09 | 354.7 | 0.9 | 60.4 | 1.7 | 5.9 | 0.5 | 8.75 |
| | 26151 | A_A237M_strat1 | 4.4E−09 | 1.5E−08 | 327.0 | 0.8 | 21.2 | 0.6 | 15.4 | 1.3 | 39.02 |
| | 26152 | A_A237F_strat1 | 1.5E−09 | 3.8E−09 | 962.2 | 2.3 | 80.4 | 2.3 | 12.0 | 1.0 | 28.33 |
| | 26153 | A_A237W_strat1 | 2.7E−09 | 4.7E−09 | 542.1 | 1.3 | 65.5 | 1.9 | 8.3 | 0.7 | 33.23 |
| | 26154 | A_A237Y_strat1 | 4.7E−09 | 1.4E−08 | 310.5 | 0.7 | 22.7 | 0.6 | 13.7 | 1.2 | 30.89 |
| | 26155 | A_A237T_strat1 | 8.4E−09 | 2.4E−08 | 172.9 | 0.4 | 13.0 | 0.4 | 13.3 | 1.1 | 41.98 |
| | 26156 | A_A237S_strat1 | 6.3E−09 | 1.9E−08 | 229.0 | 0.6 | 16.1 | 0.5 | 14.2 | 1.2 | 43.00 |
| | 26157 | A_A237Q_strat1 | 1.3E−08 | 4.7E−08 | 107.1 | 0.3 | 6.6 | 0.2 | 16.2 | 1.4 | 45.81 |
| | 26158 | A_A237N_strat1 | 4.7E−09 | 1.9E−08 | 306.8 | 0.7 | 16.1 | 0.5 | 19.0 | 1.6 | 49.54 |
| | 26159 | A_A237D_strat1 | 7.8E−09 | 4.4E−08 | 185.2 | 0.4 | 7.0 | 0.2 | 26.3 | 2.2 | 66.58 |
| | 26160 | A_A237E_strat1 | 1.6E−08 | 6.1E−08 | 89.2 | 0.2 | 5.0 | 0.1 | 17.8 | 1.5 | 58.32 |
| | 26161 | A_A237R_strat1 | 6.6E−08 | 2.8E−07 | 21.8 | 0.1 | 1.1 | 0.0 | 20.1 | 1.7 | 88.26 |
| | 26162 | A_A237K_strat1 | 6.5E−08 | 1.5E−07 | 22.3 | 0.1 | 2.0 | 0.1 | 11.1 | 0.9 | 48.11 |
| | 26163 | A_A237H_strat1 | 6.2E−09 | 1.6E−08 | 234.9 | 0.6 | 18.9 | 0.5 | 12.4 | 1.1 | 41.50 |
| | 26164 | A_A237P_strat1 | 3.7E−09 | 6.9E−09 | 389.3 | 0.9 | 44.8 | 1.3 | 8.7 | 0.7 | 19.58 |
| | 26165 | A_S239G_strat1 | 2.3E−09 | 1.1E−08 | 638.6 | 1.5 | 29.1 | 0.8 | 22.0 | 1.9 | 58.37 |
| | 26166 | A_S239A_strat1 | 2.0E−09 | 8.8E−09 | 725.1 | 1.7 | 35.0 | 1.0 | 20.7 | 1.8 | 50.65 |
| | 26167 | A_S239V_strat1 | 2.6E−09 | 8.2E−09 | 562.1 | 1.4 | 37.6 | 1.1 | 15.0 | 1.3 | 48.98 |
| | 26168 | A_S239L_strat1 | 2.8E−09 | 9.3E−09 | 510.7 | 1.2 | 33.3 | 0.9 | 15.4 | 1.3 | 55.38 |
| | 26169 | A_S239I_strat1 | 2.9E−09 | 1.0E−08 | 501.6 | 1.2 | 31.0 | 0.9 | 16.2 | 1.4 | 49.21 |
| | 26170 | A_S239M_strat1 | 3.2E−09 | 1.4E−08 | 452.5 | 1.1 | 22.1 | 0.6 | 20.4 | 1.7 | 42.16 |
| | 26171 | A_S239F_strat1 | 3.1E−09 | 1.1E−08 | 466.9 | 1.1 | 29.1 | 0.8 | 16.0 | 1.4 | 64.44 |
| | 26172 | A_S239W_strat1 | 5.4E−09 | 9.3E−09 | 269.2 | 0.6 | 33.3 | 0.9 | 8.1 | 0.7 | 33.92 |
| | 26173 | A_S239Y_strat1 | 2.5E−09 | 1.2E−08 | 575.1 | 1.4 | 25.6 | 0.7 | 22.5 | 1.9 | 50.12 |
| | 26174 | A_S239T_strat1 | 1.9E−09 | 8.4E−09 | 765.0 | 1.8 | 36.6 | 1.0 | 20.9 | 1.8 | 50.17 |
| | 26175 | A_S239Q_strat1 | 2.9E−09 | 1.1E−08 | 497.0 | 1.2 | 28.6 | 0.8 | 17.4 | 1.5 | 52.83 |
| | 26176 | A_S239N_strat1 | 2.4E−09 | 8.5E−09 | 590.3 | 1.4 | 36.1 | 1.0 | 16.3 | 1.4 | |
| | 26177 | A_S239D_strat1 | 3.9E−09 | 1.3E−08 | 369.5 | 0.9 | 23.0 | 0.7 | 16.1 | 1.4 | 50.82 |
| | 26178 | A_S239E_strat1 | 3.8E−09 | 7.3E−09 | 383.1 | 0.9 | 42.5 | 1.2 | 9.0 | 0.8 | 57.18 |
| | 26179 | A_S239R_strat1 | 4.5E−09 | 1.4E−08 | 317.8 | 0.8 | 22.8 | 0.6 | 13.9 | 1.2 | 51.40 |
| | 26180 | A_S239K_strat1 | 7.8E−09 | 2.0E−08 | 186.1 | 0.4 | 15.4 | 0.4 | 12.1 | 1.0 | 64.01 |
| | 26181 | A_S239H_strat1 | 3.1E−09 | 1.4E−08 | 463.9 | 1.1 | 22.1 | 0.6 | 21.0 | 1.8 | 95.04 |
| | 26182 | A_S239P_strat1 | 2.1E−08 | 7.6E−08 | 68.6 | 0.2 | 4.1 | 0.1 | 16.9 | 1.4 | |
| Strat1- | 26183 | B_L234G_strat1 | 3.5E−09 | 8.7E−09 | 418.2 | 1.0 | 35.4 | 1.0 | 11.8 | 1.0 | 77.33 |
| Chain B | 26184 | B_L234A_strat1 | 4.6E−09 | 1.3E−08 | 314.1 | 0.8 | 24.3 | 0.7 | 12.9 | 1.1 | 81.76 |
| | 26185 | B_L234V_strat1 | 6.0E−09 | 1.3E−08 | 239.8 | 0.6 | 24.1 | 0.7 | 10.0 | 0.8 | 70.28 |
| | 26187 | B_L234I_strat1 | 5.3E−09 | 1.2E−08 | 274.2 | 0.7 | 24.8 | 0.7 | 11.1 | 0.9 | 61.52 |
| | 26188 | B_L234M_strat1 | 4.1E−09 | 1.2E−08 | 349.2 | 0.8 | 25.6 | 0.7 | 13.6 | 1.2 | 73.71 |
| | 26189 | B_L234F_strat1 | 4.6E−09 | 9.6E−09 | 311.5 | 0.7 | 32.2 | 0.9 | 9.7 | 0.8 | 57.76 |
| | 26190 | B_L234W_strat1 | 5.3E−09 | 1.0E−08 | 270.4 | 0.7 | 30.5 | 0.9 | 8.9 | 0.8 | 38.39 |
| | 26191 | B_L234Y_strat1 | 5.4E−09 | 1.2E−08 | 268.7 | 0.6 | 25.9 | 0.7 | 10.4 | 0.9 | 46.77 |
| | 26192 | B_L234T_strat1 | 5.7E−09 | 1.3E−08 | 253.9 | 0.6 | 23.4 | 0.7 | 10.8 | 0.9 | 68.56 |
| | 26193 | B_L234S_strat1 | 5.3E−09 | 1.2E−08 | 270.3 | 0.7 | 26.5 | 0.8 | 10.2 | 0.9 | 81.06 |
| | 26194 | B_L234Q_strat1 | 4.8E−09 | 1.2E−08 | 299.4 | 0.7 | 26.0 | 0.7 | 11.5 | 1.0 | 79.51 |
| | 26195 | B_L234N_strat1 | 4.5E−09 | 1.1E−08 | 322.8 | 0.8 | 27.9 | 0.8 | 11.6 | 1.0 | 76.26 |
| | 26196 | B_L234D_strat1 | 8.2E−09 | 2.1E−08 | 175.5 | 0.4 | 14.8 | 0.4 | 11.8 | 1.0 | 76.05 |
| | 26197 | B_L234E_strat1 | 6.0E−09 | 1.5E−08 | 240.9 | 0.6 | 20.3 | 0.6 | 11.9 | 1.0 | 77.03 |
| | 26198 | B_L234R_strat1 | 1.1E−08 | 3.3E−08 | 132.2 | 0.3 | 9.3 | 0.3 | 14.1 | 1.2 | 73.62 |
| | 26199 | B_L234K_strat1 | 5.2E−09 | 1.7E−08 | 280.2 | 0.7 | 18.2 | 0.5 | 15.4 | 1.3 | 72.47 |
| | 26200 | B_L234H_strat1 | 4.6E−09 | 1.1E−08 | 315.7 | 0.8 | 28.9 | 0.8 | 10.9 | 0.9 | 66.46 |
| | 26201 | B_L234P_strat1 | 4.1E−09 | 1.1E−08 | 355.4 | 0.9 | 29.3 | 0.8 | 12.1 | 1.0 | 83.57 |
| | 26202 | B_L235G_strat1 | 5.3E−09 | 1.4E−08 | 271.4 | 0.7 | 22.6 | 0.6 | 12.0 | 1.0 | 76.27 |
| | 26203 | B_L235A_strat1 | 6.1E−09 | 1.6E−08 | 236.0 | 0.6 | 19.3 | 0.5 | 12.2 | 1.0 | 71.72 |
| | 26204 | B_L235V_strat1 | 1.0E−08 | 2.2E−08 | 142.1 | 0.3 | 14.2 | 0.4 | 10.0 | 0.8 | 47.17 |
| | 26205 | B_L235I_strat1 | 7.0E−09 | 1.5E−08 | 205.5 | 0.5 | 21.0 | 0.6 | 9.8 | 0.8 | 48.97 |
| | 26206 | B_L235M_strat1 | 4.1E−09 | 9.2E−09 | 355.7 | 0.9 | 33.6 | 1.0 | 10.6 | 0.9 | 70.65 |
| | 26207 | B_L235F_strat1 | 4.6E−09 | 9.2E−09 | 312.0 | 0.8 | 33.4 | 1.0 | 9.3 | 0.8 | 46.12 |
| | 26208 | B_L235W_strat1 | 4.0E−09 | 9.8E−09 | 360.2 | 0.9 | 31.5 | 0.9 | 11.4 | 1.0 | 60.12 |
| | 26209 | B_L235Y_strat1 | 5.0E−09 | 1.0E−08 | 287.1 | 0.7 | 30.4 | 0.9 | 9.5 | 0.8 | 56.47 |
| | 26210 | B_L235T_strat1 | 7.1E−09 | 1.6E−08 | 202.9 | 0.5 | 18.9 | 0.5 | 10.7 | 0.9 | 69.61 |
| | 26211 | B_L235S_strat1 | 5.8E−09 | 1.6E−08 | 250.1 | 0.6 | 19.2 | 0.5 | 13.0 | 1.1 | 76.35 |
| | 26212 | B_L235Q_strat1 | 7.6E−09 | 1.7E−08 | 189.0 | 0.5 | 17.7 | 0.5 | 10.7 | 0.9 | 66.97 |
| | 26213 | B_L235N_strat1 | 3.8E−09 | 1.0E−08 | 382.6 | 0.9 | 30.2 | 0.9 | 12.7 | 1.1 | 82.00 |
| | 26214 | B_L235D_strat1 | 4.3E−09 | 1.2E−08 | 336.1 | 0.8 | 25.2 | 0.7 | 13.3 | 1.1 | 89.58 |
| | 26215 | B_L235E_strat1 | 7.2E−09 | 1.7E−08 | 201.0 | 0.5 | 17.7 | 0.5 | 11.4 | 1.0 | 65.38 |
| | 26216 | B_L235R_strat1 | 3.4E−08 | 9.0E−08 | 42.5 | 0.1 | 3.4 | 0.1 | 12.3 | 1.0 | 56.28 |
| | 26217 | B_L235K_strat1 | 1.2E−08 | 2.8E−08 | 118.2 | 0.3 | 11.1 | 0.3 | 10.7 | 0.9 | 66.12 |
| | 26218 | B_L235H_strat1 | 1.1E−08 | 2.4E−08 | 130.7 | 0.3 | 13.1 | 0.4 | 10.0 | 0.8 | 75.30 |
| | 26219 | B_L235P_strat1 | 1.0E−08 | 2.1E−08 | 141.4 | 0.3 | 15.0 | 0.4 | 9.4 | 0.8 | 44.43 |
| | 26220 | B_D236G_strat1 | 1.2E−09 | 1.3E−09 | 1196.3 | 2.9 | 241.9 | 6.9 | 4.9 | 0.4 | |
| | 26221 | B_D236A_strat1 | 1.9E−09 | 1.3E−09 | 771.2 | 1.9 | 246.4 | 7.0 | 3.1 | 0.3 | 8.27 |
| | 26222 | B_D236V_strat1 | 1.3E−09 | 9.1E−10 | 1108.3 | 2.7 | 338.3 | 9.6 | 3.3 | 0.3 | 8.02 |
| | 26223 | B_D236L_strat1 | 2.2E−09 | 2.5E−09 | 654.8 | 1.6 | 122.4 | 3.5 | 5.4 | 0.5 | 10.83 |
| | 26224 | B_D236I_strat1 | 1.2E−09 | 1.1E−09 | 1194.4 | 2.9 | 274.9 | 7.8 | 4.3 | 0.4 | 9.13 |
| | 26225 | B_D236M_strat1 | 2.1E−09 | 2.4E−09 | 675.6 | 1.6 | 127.6 | 3.6 | 5.3 | 0.4 | 25.39 |
| | 26226 | B_D236F_strat1 | 2.7E−09 | 2.1E−09 | 527.6 | 1.3 | 143.7 | 4.1 | 3.7 | 0.3 | 8.02 |
| | 26227 | B_D236W_strat1 | 1.9E−09 | 9.2E−10 | 751.9 | 1.8 | 336.2 | 9.6 | 2.2 | 0.2 | 4.88 |
| | 26228 | B_D236Y_strat1 | 2.1E−09 | 2.1E−09 | 692.1 | 1.7 | 146.4 | 4.2 | 4.7 | 0.4 | 12.44 |
| | 26229 | B_D236T_strat1 | 2.0E−09 | 3.4E−09 | 715.1 | 1.7 | 91.7 | 2.6 | 7.8 | 0.7 | 40.38 |
| | 26230 | B_D236S_strat1 | 1.9E−09 | 1.8E−09 | 751.1 | 1.8 | 174.1 | 4.9 | 4.3 | 0.4 | 19.19 |
| | 26231 | B_D236Q_strat1 | 2.1E−09 | 2.3E−09 | 683.3 | 1.6 | 131.7 | 3.7 | 5.2 | 0.4 | 21.49 |
| | 26232 | B_D236N_strat1 | 3.2E−09 | 7.6E−09 | 446.9 | 1.1 | 40.8 | 1.2 | 10.9 | 0.9 | 67.16 |
| | 26233 | B_D236E_strat1 | 2.5E−09 | 3.8E−09 | 586.3 | 1.4 | 80.8 | 2.3 | 7.3 | 0.6 | 37.72 |
| | 26234 | B_D236R_strat1 | 3.2E−08 | 6.4E−08 | 45.4 | 0.1 | 4.8 | 0.1 | 9.4 | 0.8 | 58.16 |
| | 26235 | B_D236K_strat1 | 4.2E−09 | 1.8E−08 | 345.7 | 0.8 | 17.4 | 0.5 | 19.8 | 1.7 | |
| | 26236 | B_D236H_strat1 | 4.1E−09 | 4.1E−09 | 350.6 | 0.8 | 74.5 | 2.1 | 4.7 | 0.4 | |
| | 26237 | B_D236P_strat1 | 5.4E−09 | 3.7E−09 | 268.0 | 0.6 | 83.8 | 2.4 | 3.2 | 0.3 | 8.26 |
| | 26238 | B_F237G_strat1 | 9.1E−09 | 1.6E−08 | 158.2 | 0.4 | 19.6 | 0.6 | 8.1 | 0.7 | 61.39 |
| | 26239 | B_F237A_strat1 | 7.0E−09 | 1.4E−08 | 207.2 | 0.5 | 22.7 | 0.6 | 9.1 | 0.8 | 68.41 |
| | 26240 | B_F237V_strat1 | 4.3E−09 | 1.0E−08 | 333.2 | 0.8 | 30.2 | 0.9 | 11.0 | 0.9 | 69.11 |
| | 26241 | B_F237L_strat1 | 1.6E−09 | 4.7E−09 | 912.4 | 2.2 | 65.7 | 1.9 | 13.9 | 1.2 | 73.03 |
| | 26242 | B_F237I_strat1 | 4.3E−09 | 9.6E−09 | 333.6 | 0.8 | 32.0 | 0.9 | 10.4 | 0.9 | 77.30 |
| | 26243 | B_F237M_strat1 | 2.4E−09 | 5.6E−09 | 612.4 | 1.5 | 54.6 | 1.6 | 11.2 | 0.9 | 73.17 |
| | 26244 | B_F237W_strat1 | 7.0E−09 | 1.0E−08 | 206.5 | 0.5 | 30.3 | 0.9 | 6.8 | 0.6 | 53.39 |
| | 26245 | B_F237Y_strat1 | 4.7E−09 | 1.1E−08 | 307.5 | 0.7 | 27.8 | 0.8 | 11.1 | 0.9 | 78.63 |
| | 26246 | B_F237T_strat1 | 5.3E−09 | 1.2E−08 | 271.2 | 0.7 | 24.8 | 0.7 | 11.0 | 0.9 | 78.11 |
| | 26247 | B_F237S_strat1 | 9.1E−09 | 1.7E−08 | 159.5 | 0.4 | 17.9 | 0.5 | 8.9 | 0.8 | 71.06 |
| | 26248 | B_F237Q_strat1 | 5.6E−09 | 1.4E−08 | 256.1 | 0.6 | 22.4 | 0.6 | 11.4 | 1.0 | 80.41 |
| | 26249 | B_F237N_strat1 | 8.8E−09 | 2.2E−08 | 164.7 | 0.4 | 14.3 | 0.4 | 11.5 | 1.0 | 90.24 |
| | 26250 | B_F237D_strat1 | 1.4E−08 | 3.8E−08 | 101.1 | 0.2 | 8.1 | 0.2 | 12.4 | 1.1 | 74.92 |
| | 26251 | B_F237E_strat1 | 1.3E−08 | 3.3E−08 | 109.7 | 0.3 | 9.5 | 0.3 | 11.6 | 1.0 | 67.52 |
| | 26252 | B_F237R_strat1 | 1.4E−08 | 3.9E−08 | 102.9 | 0.2 | 7.9 | 0.2 | 12.9 | 1.1 | 80.19 |
| | 26253 | B_F237K_strat1 | 3.8E−09 | 7.4E−09 | 381.7 | 0.9 | 41.4 | 1.2 | 9.2 | 0.8 | 80.58 |
| | 26254 | B_F237H_strat1 | 1.1E−08 | 2.9E−08 | 127.3 | 0.3 | 10.5 | 0.3 | 12.1 | 1.0 | 70.53 |
| | 26255 | B_F237P_strat1 | 2.9E−08 | 3.4E−08 | 50.4 | 0.1 | 9.0 | 0.3 | 5.6 | 0.5 | |
| | 26256 | B_D239G_strat1 | 1.1E−08 | 1.8E−08 | 126.0 | 0.3 | 17.6 | 0.5 | 7.2 | 0.6 | 42.02 |
| | 26257 | B_D239A_strat1 | 1.3E−08 | 1.9E−08 | 110.7 | 0.3 | 16.3 | 0.5 | 6.8 | 0.6 | 46.75 |
| | 26258 | B_D239V_strat1 | 1.6E−08 | 2.4E−08 | 90.2 | 0.2 | 13.0 | 0.4 | 6.9 | 0.6 | 49.55 |
| | 26259 | B_D239L_strat1 | 1.1E−08 | 1.5E−08 | 129.5 | 0.3 | 20.2 | 0.6 | 6.4 | 0.5 | 54.35 |
| | 26260 | B_D239I_strat1 | 1.3E−08 | 2.0E−08 | 111.5 | 0.3 | 15.7 | 0.4 | 7.1 | 0.6 | 43.24 |
| | 26261 | B_D239M_strat1 | 1.2E−08 | 1.6E−08 | 124.5 | 0.3 | 19.6 | 0.6 | 6.3 | 0.5 | 45.26 |
| | 26262 | B_D239F_strat1 | 1.1E−08 | 1.7E−08 | 137.1 | 0.3 | 18.4 | 0.5 | 7.5 | 0.6 | 36.50 |
| | 26263 | B_D239W_strat1 | 1.3E−08 | 2.4E−08 | 114.3 | 0.3 | 12.6 | 0.4 | 9.1 | 0.8 | 34.55 |
| | 26264 | B_D239Y_strat1 | 2.3E−08 | 3.4E−08 | 63.8 | 0.2 | 9.0 | 0.3 | 7.1 | 0.6 | 38.63 |
| | 26265 | B_D239T_strat1 | 1.4E−08 | 2.2E−08 | 104.8 | 0.3 | 14.0 | 0.4 | 7.5 | 0.6 | 50.08 |
| | 26266 | B_D239S_strat1 | 9.9E−09 | 1.7E−08 | 145.4 | 0.4 | 18.0 | 0.5 | 8.1 | 0.7 | 54.24 |
| | 26267 | B_D239Q_strat1 | 1.7E−08 | 2.6E−08 | 85.0 | 0.2 | 11.7 | 0.3 | 7.3 | 0.6 | 40.35 |
| | 26268 | B_D239N_strat1 | 1.3E−08 | 2.3E−08 | 112.5 | 0.3 | 13.3 | 0.4 | 8.4 | 0.7 | 51.07 |
| | 26269 | B_D239E_strat1 | 5.4E−09 | 1.0E−08 | 268.8 | 0.6 | 30.3 | 0.9 | 8.9 | 0.8 | 55.82 |
| | 26270 | B_D239R_strat1 | 4.2E−08 | 7.7E−08 | 34.2 | 0.1 | 4.0 | 0.1 | 8.6 | 0.7 | 38.66 |
| | 26271 | B_D239K_strat1 | 1.1E−07 | 1.5E−07 | 13.3 | 0.0 | 2.1 | 0.1 | 6.3 | 0.5 | 27.79 |
| | 26272 | B_D239H_strat1 | 6.7E−08 | 8.5E−08 | 21.6 | 0.1 | 3.6 | 0.1 | 6.0 | 0.5 | 34.72 |
| | 26273 | B_D239P_strat1 | ND⁵ | ND | — | — | — | — | — | — | — |
| | 26274 | B_V240A_strat1 | 7.6E−09 | 1.8E−08 | 191.2 | 0.5 | 17.5 | 0.5 | 10.9 | 0.9 | 52.79 |
| | 26275 | B_V240L_strat1 | 4.9E−09 | 1.4E−08 | 297.4 | 0.7 | 22.4 | 0.6 | 13.3 | 1.1 | 71.78 |
| | 26276 | B_V240I_strat1 | 5.6E−09 | 1.3E−08 | 260.2 | 0.6 | 22.9 | 0.7 | 11.3 | 1.0 | 72.43 |
| | 26277 | B_V240M_strat1 | 5.2E−09 | 1.1E−08 | 276.3 | 0.7 | 27.8 | 0.8 | 9.9 | 0.8 | 66.30 |
| | 26278 | B_V240F_strat1 | 7.3E−08 | 9.0E−08 | 19.7 | 0.0 | 3.4 | 0.1 | 5.8 | 0.5 | 23.33 |
| | 26279 | B_V240T_strat1 | 7.5E−09 | 1.7E−08 | 191.6 | 0.5 | 18.7 | 0.5 | 10.2 | 0.9 | 64.50 |
| | 26280 | B_V263A_strat1 | 7.5E−09 | 1.6E−08 | 193.0 | 0.5 | 18.9 | 0.5 | 10.2 | 0.9 | 60.80 |
| | 26281 | B_V263L_strat1 | 1.8E−08 | 3.7E−08 | 82.3 | 0.2 | 8.2 | 0.2 | 10.0 | 0.8 | 58.34 |
| | 26282 | B_V263I_strat1 | 9.8E−09 | 3.0E−08 | 148.1 | 0.4 | 10.2 | 0.3 | 14.6 | 1.2 | 63.02 |
| | 26283 | B_V263M_strat1 | 6.6E−08 | 1.2E−07 | 21.9 | 0.1 | 2.6 | 0.1 | 8.5 | 0.7 | 44.28 |
| | 26284 | B_V263F_strat1 | ND | ND | — | — | — | — | — | — | −15.26 |
| | 26285 | B_V263T_strat1 | 4.2E−09 | 9.3E−09 | 340.5 | 0.8 | 33.3 | 0.9 | 10.2 | 0.9 | 62.70 |
| | 26286 | B_V264A_strat1 | 3.3E−08 | 8.5E−08 | 44.1 | 0.1 | 3.6 | 0.1 | 12.1 | 1.0 | 56.92 |
| | 26287 | B_V264L_strat1 | 8.3E−09 | 1.9E−08 | 175.2 | 0.4 | 15.9 | 0.5 | 11.0 | 0.9 | 58.26 |
| | 26288 | B_V264I_strat1 | 9.0E−09 | 1.9E−08 | 161.2 | 0.4 | 16.4 | 0.5 | 9.8 | 0.8 | 52.07 |
| | 26289 | B_V264M_strat1 | 4.0E−08 | 6.7E−08 | 35.9 | 0.1 | 4.6 | 0.1 | 7.8 | 0.7 | 43.82 |
| | 26290 | B_V264F_strat1 | 4.5E−08 | 9.2E−08 | 31.9 | 0.1 | 3.3 | 0.1 | 9.5 | 0.8 | 43.68 |
| | 26291 | B_V264T_strat1 | 4.9E−09 | 1.1E−08 | 295.5 | 0.7 | 27.7 | 0.8 | 10.7 | 0.9 | 69.14 |
| | 26292 | B_V266A_strat1 | 3.9E−08 | 8.5E−08 | 36.9 | 0.1 | 3.6 | 0.1 | 10.1 | 0.9 | 44.51 |
| | 26293 | B_V266L_strat1 | 8.2E−09 | 2.4E−08 | 175.2 | 0.4 | 13.0 | 0.4 | 13.4 | 1.1 | 71.22 |
| | 26294 | B_V266I_strat1 | 6.4E−09 | 1.7E−08 | 224.7 | 0.5 | 18.3 | 0.5 | 12.3 | 1.0 | 63.21 |
| | 26295 | B_V266M_strat1 | 8.8E−09 | 2.3E−08 | 163.9 | 0.4 | 13.7 | 0.4 | 12.0 | 1.0 | 78.06 |
| | 26296 | B_V266F_strat1 | 4.1E−07 | 4.6E−07 | 3.6 | 0.0 | 0.7 | 0.0 | 5.3 | 0.4 | 26.39 |
| | 26297 | B_V266T_strat1 | 5.8E−08 | 1.1E−07 | 24.9 | 0.1 | 2.8 | 0.1 | 8.9 | 0.8 | 57.20 |
| | 26298 | B_V267G_strat1 | 2.6E−07 | 6.1E−07 | 5.6 | 0.0 | 0.5 | 0.0 | 11.1 | 0.9 | 33.66 |
| | 26299 | B_V267A_strat1 | 1.8E−08 | 4.1E−08 | 78.8 | 0.2 | 7.6 | 0.2 | 10.4 | 0.9 | 48.75 |
| | 26300 | B_V267L_strat1 | 2.6E−08 | 7.6E−08 | 55.9 | 0.1 | 4.1 | 0.1 | 13.8 | 1.2 | 56.16 |
| | 26301 | B_V267I_strat1 | 8.4E−09 | 2.2E−08 | 171.9 | 0.4 | 14.0 | 0.4 | 12.3 | 1.0 | 53.65 |
| | 26302 | B_V267M_strat1 | 2.8E−09 | 4.7E−09 | 522.9 | 1.3 | 66.1 | 1.9 | 7.9 | 0.7 | 64.45 |
| | 26303 | B_V267F_strat1 | 5.2E−07 | 9.7E−07 | 2.8 | 0.0 | 0.3 | 0.0 | 8.6 | 0.7 | 52.43 |
| | 26304 | B_V267W_strat1 | 8.1E−07 | 1.4E−06 | 1.8 | 0.0 | 0.2 | 0.0 | 8.4 | 0.7 | 108.09 |
| | 26305 | B_V267Y_strat1 | 4.3E−07 | 9.2E−07 | 3.4 | 0.0 | 0.3 | 0.0 | 10.1 | 0.9 | 56.35 |
| | 26306 | B_V267T_strat1 | 2.2E−08 | 4.8E−08 | 65.1 | 0.2 | 6.4 | 0.2 | 10.1 | 0.9 | 57.29 |
| | 26307 | B_V267S_strat1 | 2.6E−08 | 7.4E−08 | 55.2 | 0.1 | 4.2 | 0.1 | 13.2 | 1.1 | 54.09 |
| | 26308 | B_V267Q_strat1 | 1.4E−09 | 2.6E−09 | 1000.1 | 2.4 | 120.6 | 3.4 | 8.3 | 0.7 | 30.86 |
| | 26309 | B_V267N_strat1 | 1.5E−07 | 3.7E−07 | 9.3 | 0.0 | 0.8 | 0.0 | 11.2 | 0.9 | 68.40 |
| | 26310 | B_V267R_strat1 | 8.2E−07 | 1.4E−06 | 1.8 | 0.0 | 0.2 | 0.0 | 8.2 | 0.7 | 605.33 |
| | 26311 | B_V267K_strat1 | ND | ND | — | — | — | — | — | — | 193.04 |
| | 26312 | B_V267H_strat1 | 1.7E−07 | 4.9E−07 | 8.3 | 0.0 | 0.6 | 0.0 | 13.1 | 1.1 | 82.01 |
| | 26313 | B_V267P_strat1 | ND | ND | — | — | — | — | — | — | 39.84 |
| | 26314 | B_D268G_strat1 | 2.1E−08 | 4.9E−08 | 69.8 | 0.2 | 6.3 | 0.2 | 11.1 | 0.9 | 66.56 |
| | 26315 | B_D268A_strat1 | 3.6E−09 | 8.6E−09 | 400.9 | 1.0 | 36.0 | 1.0 | 11.1 | 0.9 | 59.13 |
| | 26316 | B_D268V_strat1 | 3.6E−09 | 8.9E−09 | 400.1 | 1.0 | 34.8 | 1.0 | 11.5 | 1.0 | 66.95 |
| | 26317 | B_D268L_strat1 | 4.2E−09 | 9.3E−09 | 341.0 | 0.8 | 33.1 | 0.9 | 10.3 | 0.9 | 65.74 |
| | 26318 | B_D268I_strat1 | 3.4E−09 | 7.2E−09 | 422.4 | 1.0 | 43.0 | 1.2 | 9.8 | 0.8 | 62.76 |
| | 26319 | B_D268M_strat1 | 3.7E−09 | 1.0E−08 | 392.6 | 0.9 | 30.8 | 0.9 | 12.7 | 1.1 | 66.66 |
| | 26320 | B_D268F_strat1 | 2.5E−09 | 6.5E−09 | 568.1 | 1.4 | 47.5 | 1.3 | 12.0 | 1.0 | 68.88 |
| | 26321 | B_D268W_strat1 | 4.2E−09 | 8.4E−09 | 341.3 | 0.8 | 36.9 | 1.0 | 9.3 | 0.8 | 67.74 |
| | 26322 | B_D268Y_strat1 | 2.9E−09 | 6.1E−09 | 506.4 | 1.2 | 50.6 | 1.4 | 10.0 | 0.8 | 62.65 |
| | 26323 | B_D268T_strat1 | 6.1E−09 | 1.4E−08 | 238.8 | 0.6 | 21.4 | 0.6 | 11.2 | 0.9 | 60.61 |
| | 26324 | B_D268S_strat1 | 7.1E−09 | 1.7E−08 | 202.5 | 0.5 | 18.2 | 0.5 | 11.1 | 0.9 | 63.85 |
| | 26325 | B_D268Q_strat1 | 3.4E−09 | 7.2E−09 | 425.4 | 1.0 | 42.6 | 1.2 | 10.0 | 0.8 | 50.04 |
| | 26326 | B_D268N_strat1 | 6.1E−09 | 1.2E−08 | 238.6 | 0.6 | 24.7 | 0.7 | 9.6 | 0.8 | 58.98 |
| | 26327 | B_D268E_strat1 | 2.3E−09 | 7.0E−09 | 627.0 | 1.5 | 44.3 | 1.3 | 14.2 | 1.2 | 68.47 |
| | 26328 | B_D268R_strat1 | 1.2E−08 | 2.0E−08 | 125.7 | 0.3 | 15.2 | 0.4 | 8.3 | 0.7 | 44.83 |
| | 26329 | B_D268K_strat1 | 5.2E−09 | 8.4E−09 | 278.2 | 0.7 | 36.6 | 1.0 | 7.6 | 0.6 | 45.41 |
| | 26330 | B_D268H_strat1 | 1.3E−08 | 2.4E−08 | 111.0 | 0.3 | 12.8 | 0.4 | 8.7 | 0.7 | 65.59 |
| | 26331 | B_D268P_strat1 | 3.6E−09 | 8.0E−09 | 403.6 | 1.0 | 38.3 | 1.1 | 10.5 | 0.9 | 58.36 |
| | 26332 | B_E269G_strat1 | 2.2E−08 | 4.7E−08 | 64.6 | 0.2 | 6.5 | 0.2 | 9.9 | 0.8 | 46.46 |
| | 26333 | B_E269A_strat1 | 1.2E−08 | 2.6E−08 | 122.1 | 0.3 | 12.0 | 0.3 | 10.2 | 0.9 | 60.56 |
| | 26334 | B_E269V_strat1 | 1.6E−08 | 3.9E−08 | 88.7 | 0.2 | 7.9 | 0.2 | 11.2 | 0.9 | 48.60 |
| | 26335 | B_E269L_strat1 | 8.6E−09 | 2.1E−08 | 167.9 | 0.4 | 14.4 | 0.4 | 11.7 | 1.0 | 63.90 |
| | 26336 | B_E269I_strat1 | 4.1E−08 | 8.8E−08 | 34.9 | 0.1 | 3.5 | 0.1 | 9.9 | 0.8 | 39.65 |
| | 26337 | B_E269M_strat1 | 1.0E−08 | 2.1E−08 | 143.8 | 0.3 | 14.4 | 0.4 | 10.0 | 0.8 | 54.85 |
| | 26338 | B_E269F_strat1 | 2.1E−08 | 4.7E−08 | 69.3 | 0.2 | 6.5 | 0.2 | 10.6 | 0.9 | 72.90 |
| | 26339 | B_E269W_strat1 | 2.6E−08 | 8.0E−08 | 55.2 | 0.1 | 3.9 | 0.1 | 14.3 | 1.2 | 52.30 |
| | 26340 | B_E269Y_strat1 | 1.6E−08 | 5.0E−08 | 88.1 | 0.2 | 6.1 | 0.2 | 14.4 | 1.2 | 62.83 |
| | 26341 | B_E269T_strat1 | 2.0E−08 | 5.3E−08 | 70.5 | 0.2 | 5.8 | 0.2 | 12.1 | 1.0 | 29.86 |
| | 26342 | B_E269S_strat1 | 1.5E−08 | 4.3E−08 | 96.9 | 0.2 | 7.1 | 0.2 | 13.6 | 1.2 | 42.68 |
| | 26343 | B_E269Q_strat1 | 5.7E−09 | 1.8E−08 | 253.3 | 0.6 | 17.5 | 0.5 | 14.5 | 1.2 | 57.00 |
| | 26344 | B_E269N_strat1 | 1.2E−08 | 3.6E−08 | 125.0 | 0.3 | 8.6 | 0.2 | 14.6 | 1.2 | 55.35 |
| | 26345 | B_E269D_strat1 | 7.0E−09 | 2.3E−08 | 206.2 | 0.5 | 13.4 | 0.4 | 15.3 | 1.3 | 57.67 |
| | 26346 | B_E269R_strat1 | 2.7E−08 | 7.2E−08 | 54.2 | 0.1 | 4.3 | 0.1 | 12.7 | 1.1 | 53.21 |
| | 26347 | B_E269K_strat1 | 1.3E−08 | 3.7E−08 | 107.7 | 0.3 | 8.2 | 0.2 | 13.1 | 1.1 | 52.28 |
| | 26348 | B_E269H_strat1 | 1.9E−08 | 5.7E−08 | 77.7 | 0.2 | 5.4 | 0.2 | 14.3 | 1.2 | 52.83 |
| | 26349 | B_E269P_strat1 | 1.1E−07 | 3.1E−07 | 13.6 | 0.0 | 1.0 | 0.0 | 13.6 | 1.2 | 58.90 |
| | 26350 | B_D270G_strat1 | ND | ND | — | — | — | — | — | — | −45.36 |
| | 26351 | B_D270A_strat1 | ND | ND | — | — | — | — | — | — | −131.31 |
| | 26352 | B_D270V_strat1 | ND | ND | — | — | — | — | — | — | −29.42 |
| | 26353 | B_D270L_strat1 | ND | ND | — | — | — | — | — | — | −36.34 |
| | 26354 | B_D270I_strat1 | ND | ND | — | — | — | — | — | — | −26.25 |
| | 26355 | B_D270M_strat1 | ND | ND | — | — | — | — | — | — | 993.51 |
| | 26356 | B_D270F_strat1 | ND | ND | — | — | — | — | — | — | −2.53 |
| | 26357 | B_D270W_strat1 | ND | ND | — | — | — | — | — | — | −26.92 |
| | 26358 | B_D270Y_strat1 | ND | ND | — | — | — | — | — | — | −28.21 |
| | 26359 | B_D270T_strat1 | ND | ND | — | — | — | — | — | — | −50.95 |
| | 26360 | B_D270S_strat1 | ND | ND | — | — | — | — | — | — | 230.61 |
| | 26361 | B_D270Q_strat1 | ND | ND | — | — | — | — | — | — | 49.93 |
| | 26362 | B_D270N_strat1 | ND | ND | — | — | — | — | — | — | 122.82 |
| | 26363 | B_D270E_strat1 | ND | ND | — | — | — | — | — | — | 55.42 |
| | 26364 | B_D270R_strat1 | ND | ND | — | — | — | — | — | — | 2.27 |
| | 26365 | B_D270K_strat1 | ND | ND | — | — | — | — | — | — | −0.24 |
| | 26366 | B_D270H_strat1 | ND | ND | — | — | — | — | — | — | −3.29 |
| | 26367 | B_D270P_strat1 | ND | ND | — | — | — | — | — | — | 5.05 |
| | 26368 | B_P271G_strat1 | 1.4E−08 | 5.5E−08 | 105.4 | 0.3 | 5.6 | 0.2 | 18.8 | 1.6 | 65.81 |
| | 26369 | B_P271A_strat1 | 6.5E−08 | 2.4E−07 | 22.1 | 0.1 | 1.3 | 0.0 | 17.1 | 1.4 | 73.40 |
| | 26370 | B_P271V_strat1 | 3.7E−07 | 1.2E−06 | 3.9 | 0.0 | 0.3 | 0.0 | 15.2 | 1.3 | 49.62 |
| | 26371 | B_P271L_strat1 | 1.0E−07 | 3.0E−07 | 13.8 | 0.0 | 1.0 | 0.0 | 13.7 | 1.2 | 58.99 |
| | 26372 | B_P271I_strat1 | 3.6E−07 | 8.8E−07 | 4.0 | 0.0 | 0.4 | 0.0 | 11.4 | 1.0 | 46.56 |
| | 26373 | B_P271M_strat1 | 2.0E−07 | 6.5E−07 | 7.1 | 0.0 | 0.5 | 0.0 | 15.1 | 1.3 | 60.58 |
| | 26374 | B_P271F_strat1 | 2.0E−07 | 6.4E−07 | 7.1 | 0.0 | 0.5 | 0.0 | 14.5 | 1.2 | 54.91 |
| | 26375 | B_P271W_strat1 | 1.8E−07 | 5.8E−07 | 8.2 | 0.0 | 0.5 | 0.0 | 15.4 | 1.3 | 55.51 |
| | 26376 | B_P271Y_strat1 | 1.4E−07 | 4.3E−07 | 10.2 | 0.0 | 0.7 | 0.0 | 14.1 | 1.2 | 56.54 |
| | 26377 | B_P271T_strat1 | 1.3E−07 | 3.7E−07 | 10.9 | 0.0 | 0.8 | 0.0 | 13.1 | 1.1 | 64.26 |
| | 26378 | B_P271S_strat1 | 6.4E−08 | 2.2E−07 | 22.5 | 0.1 | 1.4 | 0.0 | 15.7 | 1.3 | 69.95 |
| | 26379 | B_P271Q_strat1 | 1.7E−07 | 4.9E−07 | 8.4 | 0.0 | 0.6 | 0.0 | 13.2 | 1.1 | 69.70 |
| | 26380 | B_P271N_strat1 | 6.1E−08 | 2.3E−07 | 23.6 | 0.1 | 1.4 | 0.0 | 17.4 | 1.5 | 63.65 |
| | 26381 | B_P271D_strat1 | 8.9E−09 | 3.8E−08 | 162.0 | 0.4 | 8.1 | 0.2 | 20.1 | 1.7 | 71.25 |
| | 26382 | B_P271E_strat1 | 1.3E−08 | 4.7E−08 | 113.5 | 0.3 | 6.6 | 0.2 | 17.3 | 1.5 | 64.18 |
| | 26383 | B_P271R_strat1 | 1.4E−07 | 3.7E−07 | 10.7 | 0.0 | 0.8 | 0.0 | 12.7 | 1.1 | 58.94 |
| | 26384 | B_P271K_strat1 | 5.3E−08 | 1.7E−07 | 27.0 | 0.1 | 1.8 | 0.1 | 15.0 | 1.3 | 68.31 |
| | 26385 | B_P271H_strat1 | 1.0E−07 | 3.0E−07 | 13.9 | 0.0 | 1.0 | 0.0 | 13.7 | 1.2 | 66.53 |
| | 26386 | B_V273A_strat1 | 5.3E−09 | 1.7E−08 | 273.8 | 0.7 | 17.6 | 0.5 | 15.5 | 1.3 | 71.17 |
| | 26387 | B_V273L_strat1 | 9.4E−09 | 1.9E−08 | 153.2 | 0.4 | 16.0 | 0.5 | 9.6 | 0.8 | 22.70 |
| | 26388 | B_V273I_strat1 | 3.3E−09 | 1.1E−08 | 436.5 | 1.1 | 27.4 | 0.8 | 15.9 | 1.3 | 57.65 |
| | 26389 | B_V273M_strat1 | 1.2E−08 | 2.3E−08 | 115.7 | 0.3 | 13.4 | 0.4 | 8.6 | 0.7 | 22.15 |
| | 26390 | B_V273F_strat1 | 1.7E−08 | 2.6E−08 | 83.3 | 0.2 | 11.9 | 0.3 | 7.0 | 0.6 | 9.60 |
| | 26391 | B_V273T_strat1 | 9.8E−09 | 2.8E−08 | 146.8 | 0.4 | 10.9 | 0.3 | 13.5 | 1.1 | 45.27 |
| | 26392 | B_V323A_strat1 | 3.0E−09 | 1.2E−08 | 487.9 | 1.2 | 24.7 | 0.7 | 19.8 | 1.7 | 69.62 |
| | 26393 | B_V323L_strat1 | 1.5E−08 | 4.4E−08 | 93.8 | 0.2 | 7.0 | 0.2 | 13.3 | 1.1 | 42.80 |
| | 26394 | B_V323I_strat1 | 5.4E−09 | 1.9E−08 | 269.5 | 0.6 | 15.9 | 0.5 | 17.0 | 1.4 | 64.74 |
| | 26395 | B_V323M_strat1 | 3.6E−08 | 9.4E−08 | 40.4 | 0.1 | 3.3 | 0.1 | 12.2 | 1.0 | 51.43 |
| | 26396 | B_V323F_strat1 | 1.1E−07 | 3.6E−07 | 12.7 | 0.0 | 0.9 | 0.0 | 14.9 | 1.3 | 78.06 |
| | 26397 | B_V323T_strat1 | ND | ND | — | — | — | — | — | — | — |
| | 26398 | B_S325*G_strat1 | 1.2E−08 | 3.7E−08 | 125.7 | 0.3 | 8.3 | 0.2 | 15.1 | 1.3 | 52.83 |
| | 26399 | B_S325*A_strat1 | 3.0E−09 | 1.1E−08 | 486.6 | 1.2 | 26.9 | 0.8 | 18.1 | 1.5 | 62.49 |
| | 26400 | B_S325*V_strat1 | 1.4E−07 | 1.9E−07 | 10.5 | 0.0 | 1.6 | 0.0 | 6.4 | 0.5 | 22.25 |
| | 26401 | B_S325*L_strat1 | ND | ND | | | | -- | | | 15.05 |
| | 26402 | B_S325*I_strat1 | 7.5E−08 | 9.4E−08 | 19.2 | 0.0 | 3.3 | 0.1 | 5.8 | 0.5 | 16.30 |
| | 26403 | B_S325*M_strat1 | 1.3E−08 | 1.5E−08 | 107.8 | 0.3 | 20.0 | 0.6 | 5.4 | 0.5 | 12.20 |
| | 26404 | B_S325*F_strat1 | ND | ND | — | — | — | — | — | — | 29.07 |
| | 26405 | B_S325*W_strat1 | 3.0E−08 | 2.5E−08 | 48.3 | 0.1 | 12.4 | 0.4 | 3.9 | 0.3 | 5.45 |
| | 26406 | B_S325*Y_strat1 | 1.4E−07 | 1.4E−07 | 10.5 | 0.0 | 2.2 | 0.1 | 4.7 | 0.4 | 11.19 |
| | 26407 | B_S325*T_strat1 | 1.0E−08 | 3.1E−08 | 139.1 | 0.3 | 9.9 | 0.3 | 14.1 | 1.2 | 55.54 |
| | 26408 | B_S325*Q_strat1 | 5.0E−08 | 8.4E−08 | 28.7 | 0.1 | 3.7 | 0.1 | 7.9 | 0.7 | 16.16 |
| | 26409 | B_S325*N_strat1 | 3.6E−09 | 1.2E−08 | 400.0 | 1.0 | 25.1 | 0.7 | 15.9 | 1.4 | 55.62 |
| | 26410 | B_S325*D_strat1 | 3.3E−09 | 9.4E−09 | 436.2 | 1.1 | 32.8 | 0.9 | 13.3 | 1.1 | 49.47 |
| | 26411 | B_S325*E_strat1 | 1.4E−08 | 3.7E−08 | 106.7 | 0.3 | 8.4 | 0.2 | 12.7 | 1.1 | 32.41 |
| | 26412 | B_S325*R_strat1 | 2.4E−07 | 2.5E−07 | 6.1 | 0.0 | 1.2 | 0.0 | 5.0 | 0.4 | 11.63 |
| | 26413 | B_S325*K_strat1 | 5.6E−07 | 4.5E−07 | 2.6 | 0.0 | 0.7 | 0.0 | 3.7 | 0.3 | 12.82 |
| | 26414 | B_S325*H_strat1 | 7.6E−08 | 1.2E−07 | 19.1 | 0.0 | 2.6 | 0.1 | 7.3 | 0.6 | 20.80 |
| | 26415 | B_S325*P_strat1 | 2.8E−08 | 4.0E−08 | 51.5 | 0.1 | 7.7 | 0.2 | 6.7 | 0.6 | 17.55 |
| | 26416 | B_T326*G_strat1 | 1.4E−08 | 4.6E−08 | 102.4 | 0.2 | 6.8 | 0.2 | 15.1 | 1.3 | 41.69 |
| | 26417 | B_T326*A_strat1 | 4.3E−09 | 1.6E−08 | 336.9 | 0.8 | 18.9 | 0.5 | 17.8 | 1.5 | 72.57 |
| | 26418 | B_T326*V_strat1 | 3.1E−09 | 1.3E−08 | 461.9 | 1.1 | 24.3 | 0.7 | 19.0 | 1.6 | 66.06 |
| | 26419 | B_T326*L_strat1 | 3.8E−09 | 1.6E−08 | 381.9 | 0.9 | 19.1 | 0.5 | 20.0 | 1.7 | 69.14 |
| | 26420 | B_T326*I_strat1 | 3.1E−09 | 1.2E−08 | 467.7 | 1.1 | 26.0 | 0.7 | 18.0 | 1.5 | 72.00 |
| | 26421 | B_T326*M_strat1 | 3.2E−09 | 1.3E−08 | 449.9 | 1.1 | 23.7 | 0.7 | 19.0 | 1.6 | 68.21 |
| | 26422 | B_T326*F_strat1 | 3.1E−09 | 1.2E−08 | 465.1 | 1.1 | 25.9 | 0.7 | 17.9 | 1.5 | 70.49 |
| | 26423 | B_T326*W_strat1 | 3.5E−09 | 1.4E−08 | 416.2 | 1.0 | 22.4 | 0.6 | 18.6 | 1.6 | 71.08 |
| | 26424 | B_T326*Y_strat1 | 3.9E−09 | 1.5E−08 | 373.3 | 0.9 | 21.2 | 0.6 | 17.6 | 1.5 | 81.03 |
| | 26425 | B_T326*S_strat1 | 4.7E−09 | 1.7E−08 | 304.6 | 0.7 | 17.7 | 0.5 | 17.2 | 1.5 | 69.75 |
| | 26426 | B_T326*Q_strat1 | 3.5E−09 | 1.5E−08 | 412.2 | 1.0 | 21.2 | 0.6 | 19.4 | 1.6 | 78.70 |
| | 26427 | B_T326*N_strat1 | 1.9E−09 | 7.5E−09 | 757.4 | 1.8 | 40.9 | 1.2 | 18.5 | 1.6 | 75.46 |
| | 26428 | B_T326*D_strat1 | 1.3E−09 | 5.6E−09 | 1120.3 | 2.7 | 54.6 | 1.6 | 20.5 | 1.7 | 74.15 |
| | 26429 | B_T326*E_strat1 | 1.8E−09 | 8.0E−09 | 816.9 | 2.0 | 38.7 | 1.1 | 21.1 | 1.8 | 84.13 |
| | 26430 | B_T326*R_strat1 | 9.1E−09 | 3.4E−08 | 159.3 | 0.4 | 9.0 | 0.3 | 17.7 | 1.5 | 78.00 |
| | 26431 | B_T326*K_strat1 | 7.5E−09 | 2.6E−08 | 193.4 | 0.5 | 11.6 | 0.3 | 16.6 | 1.4 | 83.61 |
| | 26432 | B_T326*H_strat1 | 2.8E−09 | 1.1E−08 | 508.0 | 1.2 | 27.4 | 0.8 | 18.5 | 1.6 | 79.14 |
| | 26433 | B_T326*P_strat1 | 2.8E−08 | 8.9E−08 | 52.4 | 0.1 | 3.5 | 0.1 | 15.1 | 1.3 | 50.82 |
| | 26434 | B_W327*G_strat1 | 5.4E−08 | 7.7E−08 | 26.5 | 0.1 | 4.0 | 0.1 | 6.6 | 0.6 | 17.30 |
| | 26435 | B_W327*A_strat1 | 5.9E−08 | 9.3E−08 | 24.4 | 0.1 | 3.3 | 0.1 | 7.4 | 0.6 | 17.12 |
| | 26436 | B_W327*V_strat1 | 7.3E−08 | 1.1E−07 | 19.8 | 0.0 | 2.9 | 0.1 | 6.8 | 0.6 | 16.05 |
| | 26437 | B_W327*L_strat1 | 6.7E−08 | 9.4E−08 | 21.5 | 0.1 | 3.3 | 0.1 | 6.5 | 0.6 | 28.17 |
| | 26438 | B_W327*I_strat1 | 8.1E−08 | 1.2E−07 | 17.9 | 0.0 | 2.7 | 0.1 | 6.7 | 0.6 | 20.52 |
| | 26439 | B_W327*M_strat1 | 7.1E−08 | 9.6E−08 | 20.4 | 0.0 | 3.2 | 0.1 | 6.3 | 0.5 | 18.60 |
| | 26440 | B_W327*F_strat1 | 2.8E−08 | 6.2E−08 | 50.8 | 0.1 | 5.0 | 0.1 | 10.2 | 0.9 | 36.44 |
| | 26441 | B_W327*Y_strat1 | 5.3E−08 | 6.7E−08 | 27.1 | 0.1 | 4.6 | 0.1 | 5.9 | 0.5 | 13.69 |
| | 26442 | B_W327*T_strat1 | 5.3E−08 | 7.6E−08 | 27.3 | 0.1 | 4.1 | 0.1 | 6.7 | 0.6 | 17.62 |
| | 26443 | B_W327*S_strat1 | 6.1E−08 | 8.5E−08 | 23.6 | 0.1 | 3.6 | 0.1 | 6.5 | 0.6 | 16.33 |
| | 26444 | B_W327*Q_strat1 | 6.4E−08 | 8.1E−08 | 22.7 | 0.1 | 3.8 | 0.1 | 5.9 | 0.5 | 14.31 |
| | 26445 | B_W327*N_strat1 | 5.6E−08 | 6.8E−08 | 25.8 | 0.1 | 4.5 | 0.1 | 5.7 | 0.5 | 78.22 |
| | 26446 | B_W327*D_strat1 | 5.6E−08 | 7.5E−08 | 25.9 | 0.1 | 4.1 | 0.1 | 6.3 | 0.5 | 15.23 |
| | 26447 | B_W327*E_strat1 | 6.4E−08 | 8.4E−08 | 22.6 | 0.1 | 3.7 | 0.1 | 6.2 | 0.5 | 15.51 |
| | 26448 | B_W327*R_strat1 | 1.4E−07 | 1.7E−07 | 10.3 | 0.0 | 1.8 | 0.1 | 5.8 | 0.5 | 15.02 |
| | 26449 | B_W327*K_strat1 | 1.7E−07 | 1.9E−07 | 8.4 | 0.0 | 1.7 | 0.0 | 5.1 | 0.4 | 15.51 |
| | 26450 | B_W327*H_strat1 | 9.0E−08 | 1.2E−07 | 16.1 | 0.0 | 2.5 | 0.1 | 6.4 | 0.5 | 15.65 |
| | 26451 | B_W327*P_strat1 | 7.3E−08 | 9.6E−08 | 19.7 | 0.0 | 3.2 | 0.1 | 6.2 | 0.5 | 15.13 |
| | 26452 | B_F328*G_strat1 | 2.8E−08 | 6.6E−08 | 51.3 | 0.1 | 4.7 | 0.1 | 11.0 | 0.9 | 35.03 |
| | 26453 | B_F328*A_strat1 | 4.1E−09 | 1.3E−08 | 352.8 | 0.8 | 23.0 | 0.7 | 15.4 | 1.3 | 70.29 |
| | 26454 | B_F328*V_strat1 | 3.2E−09 | 9.5E−09 | 451.8 | 1.1 | 32.5 | 0.9 | 13.9 | 1.2 | 55.78 |
| | 26455 | B_F328*L_strat1 | 1.2E−08 | 3.2E−08 | 125.4 | 0.3 | 9.8 | 0.3 | 12.8 | 1.1 | 51.52 |
| | 26456 | B_F328*I_strat1 | 5.9E−09 | 1.8E−08 | 244.1 | 0.6 | 17.3 | 0.5 | 14.1 | 1.2 | 56.58 |
| | 26457 | B_F328*M_strat1 | 6.6E−09 | 2.0E−08 | 218.7 | 0.5 | 15.6 | 0.4 | 14.0 | 1.2 | 59.33 |
| | 26458 | B_F328*W_strat1 | 4.2E−09 | 1.6E−08 | 345.2 | 0.8 | 19.7 | 0.6 | 17.5 | 1.5 | 69.10 |
| | 26459 | B_F328*Y_strat1 | 3.1E−09 | 1.2E−08 | 462.6 | 1.1 | 25.1 | 0.7 | 18.4 | 1.6 | 72.61 |
| | 26460 | B_F328*T_strat1 | 5.5E−09 | 1.7E−08 | 260.6 | 0.6 | 17.8 | 0.5 | 14.6 | 1.2 | 59.19 |
| | 26461 | B_F328*S_strat1 | 5.8E−09 | 2.3E−08 | 249.6 | 0.6 | 13.2 | 0.4 | 19.0 | 1.6 | |
| | 26462 | B_F328*Q_strat1 | 1.0E−08 | 2.6E−08 | 144.4 | 0.3 | 12.1 | 0.3 | 11.9 | 1.0 | 50.98 |
| | 26463 | B_F328*N_strat1 | 1.2E−08 | 3.9E−08 | 119.7 | 0.3 | 7.9 | 0.2 | 15.1 | 1.3 | 59.97 |
| | 26464 | B_F328*D_strat1 | 2.5E−08 | 5.2E−08 | 58.5 | 0.1 | 6.0 | 0.2 | 9.8 | 0.8 | 42.69 |
| | 26465 | B_F328*E_strat1 | 1.4E−08 | 3.7E−08 | 103.1 | 0.2 | 8.4 | 0.2 | 12.2 | 1.0 | 35.59 |
| | 26466 | B_F328*R_strat1 | 3.1E−08 | 8.1E−08 | 46.9 | 0.1 | 3.8 | 0.1 | 12.3 | 1.0 | 45.87 |
| | 26467 | B_F328*K_strat1 | 7.9E−09 | 2.4E−08 | 182.4 | 0.4 | 12.6 | 0.4 | 14.5 | 1.2 | 51.06 |
| | 26468 | B_F328*H_strat1 | 5.0E−09 | 2.1E−08 | 290.4 | 0.7 | 14.8 | 0.4 | 19.7 | 1.7 | 72.38 |
| | 26469 | B_F328*P_strat1 | 5.2E−08 | 6.1E−08 | 27.8 | 0.1 | 5.0 | 0.1 | 5.5 | 0.5 | 12.95 |
| | 26470 | B_D329*G_strat1 | 1.4E−09 | 5.3E−09 | 1038.9 | 2.5 | 57.8 | 1.6 | 18.0 | 1.5 | 58.96 |
| | 26471 | B_D329*A_strat1 | 1.5E−08 | 6.1E−08 | 93.4 | 0.2 | 5.0 | 0.1 | 18.5 | 1.6 | 72.31 |
| | 26472 | B_D329*V_strat1 | 2.0E−08 | 1.3E−07 | 72.8 | 0.2 | 2.4 | 0.1 | 30.5 | 2.6 | 96.22 |
| | 26473 | B_D329*L_strat1 | 4.1E−09 | 3.3E−08 | 350.4 | 0.8 | 9.5 | 0.3 | 37.0 | 3.1 | 98.00 |
| | 26474 | B_D329*I_strat1 | 5.6E−09 | 6.9E−08 | 259.4 | 0.6 | 4.5 | 0.1 | 57.7 | 4.9 | 100.87 |
| | 26475 | B_D329*M_strat1 | 3.4E−09 | 1.4E−08 | 421.8 | 1.0 | 22.0 | 0.6 | 19.2 | 1.6 | 67.09 |
| | 26476 | B_D329*F_strat1 | 3.2E−08 | 2.2E−07 | 45.3 | 0.1 | 1.4 | 0.0 | 31.7 | 2.7 | 101.20 |
| | 26477 | B_D329*W_strat1 | 2.6E−08 | 1.3E−07 | 55.6 | 0.1 | 2.3 | 0.1 | 24.0 | 2.0 | 97.32 |
| | 26478 | B_D329*Y_strat1 | 5.8E−08 | 3.9E−07 | 24.7 | 0.1 | 0.8 | 0.0 | 31.2 | 2.6 | 107.04 |
| | 26479 | B_D329*T_strat1 | 1.9E−08 | 9.9E−08 | 77.4 | 0.2 | 3.1 | 0.1 | 24.8 | 2.1 | 94.56 |
| | 26480 | B_D329*S_strat1 | 1.8E−08 | 7.4E−08 | 79.9 | 0.2 | 4.2 | 0.1 | 19.1 | 1.6 | 85.35 |
| | 26481 | B_D329*Q_strat1 | 4.2E−09 | 1.4E−08 | 348.2 | 0.8 | 22.9 | 0.6 | 15.2 | 1.3 | 52.14 |
| | 26482 | B_D329*N_strat1 | 2.0E−08 | 9.0E−08 | 71.9 | 0.2 | 3.4 | 0.1 | 21.1 | 1.8 | 79.10 |
| | 26483 | B_D329*E_strat1 | 1.6E−09 | 4.3E−09 | 914.1 | 2.2 | 71.7 | 2.0 | 12.7 | 1.1 | 38.02 |
| | 26484 | B_D329*R_strat1 | 3.7E−08 | 2.5E−07 | 39.2 | 0.1 | 1.2 | 0.0 | 31.5 | 2.7 | 98.19 |
| | 26485 | B_D329*K_strat1 | 3.3E−08 | 2.0E−07 | 44.3 | 0.1 | 1.5 | 0.0 | 29.4 | 2.5 | 77.62 |
| | 26486 | B_D329*H_strat1 | 4.7E−08 | 1.9E−07 | 30.7 | 0.1 | 1.7 | 0.0 | 18.5 | 1.6 | 76.19 |
| | 26487 | B_D329*P_strat1 | 4.5E−09 | 1.6E−08 | 322.7 | 0.8 | 18.8 | 0.5 | 17.2 | 1.5 | 79.30 |
| | 26488 | B_G330*A_strat1 | 5.1E−10 | 2.4E−09 | 2829.2 | 6.8 | 127.8 | 3.6 | 22.1 | 1.9 | 44.56 |
| | 26489 | B_G330*V_strat1 | 2.2E−10 | 3.5E−10 | 6539.2 | 15.7 | 890.3 | 25.3 | 7.3 | 0.6 | 11.19 |
| | 26490 | B_G330*L_strat1 | 8.9E−11 | 2.8E−10 | 16152.6 | 38.9 | 1103.7 | 31.4 | 14.6 | 1.2 | 20.45 |
| | 26491 | B_G330*I_strat1 | 5.8E−11 | 2.0E−10 | 24998.6 | 60.2 | 1530.2 | 43.5 | 16.3 | 1.4 | 22.39 |
| | 26492 | B_G330*M_strat1 | 5.4E−10 | 7.5E−10 | 2692.7 | 6.5 | 414.1 | 11.8 | 6.5 | 0.6 | 19.19 |
| | 26493 | B_G330*F_strat1 | 1.6E−09 | 1.8E−09 | 910.1 | 2.2 | 167.7 | 4.8 | 5.4 | 0.5 | 19.62 |
| | 26494 | B_G330*W_strat1 | 2.4E−09 | 2.8E−09 | 610.3 | 1.5 | 111.7 | 3.2 | 5.5 | 0.5 | 10.40 |
| | 26495 | B_G330*Y_strat1 | 1.7E−09 | 3.1E−09 | 861.2 | 2.1 | 100.1 | 2.8 | 8.6 | 0.7 | 15.12 |
| | 26496 | B_G330*T_strat1 | 6.7E−10 | 2.1E−09 | 2166.0 | 5.2 | 149.2 | 4.2 | 14.5 | 1.2 | 27.81 |
| | 26497 | B_G330*S_strat1 | 5.1E−10 | 1.6E−09 | 2825.4 | 6.8 | 192.8 | 5.5 | 14.7 | 1.2 | 37.09 |
| | 26498 | B_G330*Q_strat1 | 2.1E−10 | 5.1E−10 | 6944.6 | 16.7 | 605.0 | 17.2 | 11.5 | 1.0 | 38.18 |
| | 26499 | B_G330*N_strat1 | 1.1E−09 | 2.9E−09 | 1334.4 | 3.2 | 107.8 | 3.1 | 12.4 | 1.0 | 35.99 |
| | 26500 | B_G330*D_strat1 | 5.1E−09 | 3.2E−08 | 282.5 | 0.7 | 9.6 | 0.3 | 29.4 | 2.5 | 94.00 |
| | 26501 | B_G330*E_strat1 | 1.7E−09 | 6.9E−09 | 856.7 | 2.1 | 45.0 | 1.3 | 19.1 | 1.6 | 71.50 |
| | 26502 | B_G330*R_strat1 | 7.8E−09 | 3.4E−08 | 185.2 | 0.4 | 9.0 | 0.3 | 20.5 | 1.7 | 54.07 |
| | 26503 | B_G330*K_strat1 | 1.4E−09 | 1.2E−08 | 1024.9 | 2.5 | 26.5 | 0.8 | 38.7 | 3.3 | 78.74 |
| | 26504 | B_G330*H_strat1 | 9.2E−10 | 5.1E−09 | 1571.4 | 3.8 | 61.0 | 1.7 | 25.8 | 2.2 | 64.27 |
| | 26505 | B_G330*P_strat1 | 4.6E−08 | 1.1E−07 | 31.6 | 0.1 | 2.7 | 0.1 | 11.5 | 1.0 | 52.13 |
| | 26506 | B_G331*A_strat1 | 1.4E−08 | 3.6E−08 | 106.6 | 0.3 | 8.6 | 0.2 | 12.4 | 1.1 | 46.95 |
| | 26507 | B_G331*V_strat1 | 1.1E−08 | 3.3E−08 | 134.7 | 0.3 | 9.3 | 0.3 | 14.4 | 1.2 | 59.25 |
| | 26508 | B_G331*L_strat1 | 1.8E−08 | 4.3E−08 | 80.9 | 0.2 | 7.2 | 0.2 | 11.2 | 0.9 | 51.69 |
| | 26509 | B_G331*I_strat1 | 1.2E−08 | 3.8E−08 | 115.8 | 0.3 | 8.2 | 0.2 | 14.2 | 1.2 | 58.45 |
| | 26510 | B_G331*M_strat1 | 1.1E−08 | 2.9E−08 | 133.7 | 0.3 | 10.7 | 0.3 | 12.5 | 1.1 | |
| | 26511 | B_G331*F_strat1 | 1.7E−08 | 4.7E−08 | 85.1 | 0.2 | 6.6 | 0.2 | 12.9 | 1.1 | 48.43 |
| | 26512 | B_G331*W_strat1 | 2.3E−08 | 5.6E−08 | 64.0 | 0.2 | 5.5 | 0.2 | 11.7 | 1.0 | 45.10 |
| | 26513 | B_G331*Y_strat1 | 1.4E−08 | 4.2E−08 | 101.9 | 0.2 | 7.3 | 0.2 | 13.9 | 1.2 | |
| | 26514 | B_G331*T_strat1 | 1.9E−08 | 4.2E−08 | 75.4 | 0.2 | 7.3 | 0.2 | 10.3 | 0.9 | 34.34 |
| | 26515 | B_G331*S_strat1 | 1.8E−08 | 4.4E−08 | 79.0 | 0.2 | 7.0 | 0.2 | 11.3 | 1.0 | 37.11 |
| | 26516 | B_G331*Q_strat1 | 1.2E−08 | 3.4E−08 | 117.2 | 0.3 | 9.2 | 0.3 | 12.8 | 1.1 | 56.79 |
| | 26517 | B_G331*N_strat1 | 1.3E−08 | 3.3E−08 | 107.8 | 0.3 | 9.5 | 0.3 | 11.4 | 1.0 | 60.12 |
| | 26518 | B_G331*D_strat1 | 9.2E−09 | 2.5E−08 | 157.4 | 0.4 | 12.3 | 0.3 | 12.8 | 1.1 | 61.43 |
| | 26519 | B_G331*E_strat1 | 9.3E−09 | 2.9E−08 | 156.1 | 0.4 | 10.7 | 0.3 | 14.5 | 1.2 | 54.70 |
| | 26520 | B_G331*R_strat1 | 3.1E−08 | 5.3E−08 | 47.2 | 0.1 | 5.9 | 0.2 | 8.0 | 0.7 | 49.46 |
| | 26521 | B_G331*K_strat1 | 1.5E−08 | 3.8E−08 | 97.1 | 0.2 | 8.1 | 0.2 | 11.9 | 1.0 | 64.07 |
| | 26522 | B_G331*H_strat1 | 1.5E−08 | 3.8E−08 | 93.8 | 0.2 | 8.1 | 0.2 | 11.6 | 1.0 | 58.19 |
| | 26523 | B_G331*P_strat1 | 1.6E−08 | 5.0E−08 | 91.9 | 0.2 | 6.1 | 0.2 | 15.0 | 1.3 | 55.73 |
| | 26524 | B_Y331*AG_strat1 | 1.7E−08 | 3.8E−08 | 82.8 | 0.2 | 8.1 | 0.2 | 10.2 | 0.9 | 36.42 |
| | 26525 | B_Y331*AA_strat1 | 6.9E−09 | 2.2E−08 | 208.6 | 0.5 | 14.1 | 0.4 | 14.8 | 1.3 | 65.46 |
| | 26526 | B_Y331*AV_strat1 | 2.2E−08 | 5.7E−08 | 66.7 | 0.2 | 5.4 | 0.2 | 12.3 | 1.0 | 47.55 |
| | 26527 | B_Y331*AL_strat1 | 1.1E−08 | 2.8E−08 | 133.0 | 0.3 | 11.2 | 0.3 | 11.9 | 1.0 | |
| | 26528 | B_Y331*AI_strat1 | 2.0E−08 | 5.8E−08 | 73.2 | 0.2 | 5.3 | 0.2 | 13.7 | 1.2 | 46.42 |
| | 26529 | B_Y331*AM_strat1 | 1.0E−08 | 3.2E−08 | 145.0 | 0.3 | 9.5 | 0.3 | 15.3 | 1.3 | 67.84 |
| | 26530 | B_Y331*AF_strat1 | 3.0E−09 | 1.1E−08 | 489.2 | 1.2 | 27.5 | 0.8 | 17.8 | 1.5 | 71.90 |
| | 26531 | B_Y331*AW_strat1 | 3.5E−09 | 1.5E−08 | 411.0 | 1.0 | 20.9 | 0.6 | 19.7 | 1.7 | |
| | 26532 | B_Y331*AT_strat1 | 2.1E−08 | 5.2E−08 | 67.9 | 0.2 | 5.9 | 0.2 | 11.5 | 1.0 | 40.78 |
| | 26533 | B_Y331*AS_strat1 | 1.3E−08 | 3.7E−08 | 107.6 | 0.3 | 8.4 | 0.2 | 12.8 | 1.1 | 48.99 |
| | 26534 | B_Y331*AQ_strat1 | 1.3E−08 | 2.9E−08 | 112.4 | 0.3 | 10.5 | 0.3 | 10.7 | 0.9 | 55.16 |
| | 26535 | B_Y331*AD_strat1 | 4.2E−09 | 1.4E−08 | 346.5 | 0.8 | 21.6 | 0.6 | 16.1 | 1.4 | 62.44 |
| | 26536 | B_Y331*AE_strat1 | 6.6E−09 | 2.0E−08 | 219.3 | 0.5 | 15.1 | 0.4 | 14.5 | 1.2 | 63.93 |
| | 26537 | B_Y331*AR_strat1 | 1.5E−08 | 3.5E−08 | 98.9 | 0.2 | 8.7 | 0.2 | 11.3 | 1.0 | |
| | 26538 | B_Y331*AK_strat1 | 1.2E−08 | 3.7E−08 | 117.4 | 0.3 | 8.3 | 0.2 | 14.2 | 1.2 | 50.32 |
| | 26539 | B_Y331*AH_strat1 | 6.7E−09 | 2.1E−08 | 216.4 | 0.5 | 14.7 | 0.4 | 14.7 | 1.2 | 66.02 |
| | 26540 | B_Y331*AP_strat1 | 4.4E−09 | 1.4E−08 | 325.3 | 0.8 | 21.9 | 0.6 | 14.9 | 1.3 | 63.71 |
| | 26541 | B_A331*BG_strat1 | 4.4E−09 | 1.4E−08 | 329.1 | 0.8 | 22.4 | 0.6 | 14.7 | 1.2 | |
| | 26542 | B_A331*BV_strat1 | 7.6E−09 | 2.8E−08 | 190.8 | 0.5 | 10.9 | 0.3 | 17.5 | 1.5 | |
| | 26543 | B_A331*BL_strat1 | 2.4E−09 | 1.2E−08 | 596.2 | 1.4 | 25.1 | 0.7 | 23.8 | 2.0 | 82.23 |
| | 26544 | B_A331*BI_strat1 | 1.4E−08 | 4.6E−08 | 107.0 | 0.3 | 6.6 | 0.2 | 16.1 | 1.4 | 56.77 |
| | 26545 | B_A331*BM_strat1 | 2.6E−09 | 1.2E−08 | 556.7 | 1.3 | 26.7 | 0.8 | 20.8 | 1.8 | 74.11 |
| | 26546 | B_A331*BF_strat1 | 7.9E−09 | 3.0E−08 | 182.5 | 0.4 | 10.2 | 0.3 | 17.9 | 1.5 | 71.29 |
| | 26547 | B_A331*BW_strat1 | 3.0E−08 | 8.7E−08 | 48.4 | 0.1 | 3.6 | 0.1 | 13.6 | 1.2 | 49.76 |
| | 26548 | B_A331*BY_strat1 | 7.1E−09 | 2.6E−08 | 204.6 | 0.5 | 11.8 | 0.3 | 17.4 | 1.5 | 69.55 |
| | 26549 | B_A331*BT_strat1 | 7.8E−09 | 1.9E−08 | 185.0 | 0.4 | 16.0 | 0.5 | 11.6 | 1.0 | 50.19 |
| | 26550 | B_A331*BS_strat1 | 7.0E−09 | 1.8E−08 | 205.1 | 0.5 | 17.3 | 0.5 | 11.9 | 1.0 | 49.06 |
| | 26551 | B_A331*BQ_strat1 | 2.8E−09 | 1.0E−08 | 516.3 | 1.2 | 30.1 | 0.9 | 17.2 | 1.5 | 71.84 |
| | 26552 | B_A331*BN_strat1 | 4.5E−09 | 1.5E−08 | 318.0 | 0.8 | 21.2 | 0.6 | 15.0 | 1.3 | 59.55 |
| | 26553 | B_A331*BD_strat1 | 6.6E−09 | 1.7E−08 | 219.5 | 0.5 | 17.9 | 0.5 | 12.3 | 1.0 | 48.23 |
| | 26554 | B_A331*BE_strat1 | 2.8E−09 | 9.5E−09 | 518.0 | 1.2 | 32.5 | 0.9 | 15.9 | 1.3 | 65.52 |
| | 26555 | B_A331*BR_strat1 | 6.0E−09 | 1.4E−08 | 240.7 | 0.6 | 21.9 | 0.6 | 11.0 | 0.9 | 58.60 |
| | 26556 | B_A331*BK_strat1 | 2.3E−09 | 9.4E−09 | 631.6 | 1.5 | 32.8 | 0.9 | 19.3 | 1.6 | |
| | 26557 | B_A331*BH_strat1 | 3.5E−09 | 1.4E−08 | 414.4 | 1.0 | 21.6 | 0.6 | 19.2 | 1.6 | |
| | 26558 | B_A331*BP_strat1 | 6.0E−08 | 7.9E−08 | 23.9 | 0.1 | 3.9 | 0.1 | 6.1 | 0.5 | 11.95 |
| | 26559 | B_I332A_strat1 | 2.3E−08 | 2.9E−08 | 62.2 | 0.1 | 10.5 | 0.3 | 5.9 | 0.5 | |
| | 26560 | B_I332V_strat1 | 1.4E−08 | 3.3E−08 | 101.3 | 0.2 | 9.4 | 0.3 | 10.7 | 0.9 | |
| | 26561 | B_I332L_strat1 | 1.6E−09 | 9.3E−09 | 911.2 | 2.2 | 33.1 | 0.9 | 27.5 | 2.3 | 71.85 |
| | 27295 | B_I332T_strat1 | 2.5E−08 | 4.5E−08 | 58.2 | 0.1 | 6.9 | 0.2 | 8.4 | 0.7 | 289.42 |
| | 26562 | B_I332M_strat1 | 3.6E−09 | 1.2E−08 | 404.9 | 1.0 | 26.0 | 0.7 | 15.6 | 1.3 | 65.42 |
| | 26563 | B_I332F_strat1 | 3.5E−09 | 1.4E−08 | 408.1 | 1.0 | 22.8 | 0.6 | 17.9 | 1.5 | |

¹Mutation notation is in the format A_L234G_strat1, where “A” indicates the Fc chain, “L234G” indicates the mutation made with “L” representing the parental residue being replaced, 234 representing the position and G representing the replacement residue, and “strat1” specifies the parental CH2 mutations (A_G236N_G237A/B_G236D_G237F_S239D_S267V_H268D_Template1)²
²Selectivity is defined as IIb-Fold/IIaR-Fold
³% of non-competed FcγRIIb signal in presence of 10x FcγRIIa
⁴IIb-Specific Comparator: Mimoto, et al., 2013, Protein Eng. Des. Sel., 26: 589-598
⁵ND—signal too low for accurate measurement

TABLE 6.18
Strategy 2 Variants

| | | | | | | IIb- | | IIaR- | | IIb | |
|---|---|---|---|---|---|---|---|---|---|---|---|
| | | | | | | Fold | | Fold | | Selectivity² | |
| | Variant | | FcγRIIb | FcγRIIaR | IIb- | wrt | IIaR- | wrt | IIb | Fold wrt | |
| Strategy | # | Mutations¹ | K_{D} | K_{D} | Fold | Control | Fold | Control | Selectivity² | Control | ELISA³ |
| Controls | 16463 | WT | 1.4E−06 | 3.1E−07 | 1.0 | | 1.0 | | 1.0 | | |
| | 27294 | strat2_control | 1.6E−08 | 4.4E−08 | 92.9 | 1.0 | 7.0 | 1.0 | 13.4 | 1.0 | 47.77 |
| | | (A_L234F_G236N_ | | | | | | | | | |
| | | H268Q_A327G_A330K_ | | | | | | | | | |
| | | P331S | | | | | | | | | |
| | | B_G236D_S239D_ | | | | | | | | | |
| | | V266L_S267A_H268D) | | | | | | | | | |
| | v124 | Symmetrical | 1.3E−08 | 2.2E−07 | 111.6 | | 1.4 | | 80.5 | | 100.12 |
| | | E233D_G237D_P238D_ | | | | | | | | | |
| | | H268D_P271G_A330R | | | | | | | | | |
| Strat2 | 26565 | A_F234G_strat2 | 2.6E−08 | 7.6E−08 | 55.2 | 0.6 | 4.1 | 0.6 | 13.6 | 1.0 | 46.08 |
| Chain A | 26566 | A_F234A_strat2 | 1.6E−08 | 3.7E−08 | 88.4 | 1.0 | 8.3 | 1.2 | 10.7 | 0.8 | 29.61 |
| | 26567 | A_F234V_strat2 | 9.9E−09 | 2.1E−08 | 145.7 | 1.6 | 14.5 | 2.1 | 10.0 | 0.8 | 33.56 |
| | 26568 | A_F234L_strat2 | 8.5E−09 | 1.8E−08 | 169.3 | 1.8 | 16.8 | 2.4 | 10.1 | 0.8 | 26.52 |
| | 26569 | A_F234I_strat2 | 1.1E−08 | 2.1E−08 | 132.0 | 1.4 | 14.7 | 2.1 | 9.0 | 0.7 | 24.58 |
| | 26570 | A_F234W_strat2 | 9.9E−09 | 2.6E−08 | 145.8 | 1.6 | 11.9 | 1.7 | 12.2 | 0.9 | 40.98 |
| | 26571 | A_F234Y_strat2 | 2.3E−08 | 5.8E−08 | 62.4 | 0.7 | 5.3 | 0.8 | 11.7 | 0.9 | 38.48 |
| | 26572 | A_F234T_strat2 | 1.8E−08 | 4.9E−08 | 81.0 | 0.9 | 6.3 | 0.9 | 12.9 | 1.0 | 48.91 |
| | 26573 | A_F234S_strat2 | 1.6E−08 | 5.0E−08 | 89.5 | 1.0 | 6.2 | 0.9 | 14.5 | 1.1 | 53.05 |
| | 26574 | A_F234Q_strat2 | 1.9E−08 | 5.8E−08 | 77.2 | 0.8 | 5.3 | 0.8 | 14.6 | 1.1 | 55.47 |
| | 26575 | A_F234N_strat2 | 1.6E−08 | 4.9E−08 | 91.7 | 1.0 | 6.3 | 0.9 | 14.6 | 1.1 | 55.64 |
| | 26576 | A_F234D_strat2 | 3.2E−08 | 9.3E−08 | 45.0 | 0.5 | 3.3 | 0.5 | 13.6 | 1.0 | 64.15 |
| | 26577 | A_F234E_strat2 | 3.4E−08 | 5.8E−08 | 43.1 | 0.5 | 5.3 | 0.8 | 8.1 | 0.6 | 67.28 |
| | 26578 | A_F234R_strat2 | 4.8E−08 | 1.2E−07 | 29.9 | 0.3 | 2.6 | 0.4 | 11.4 | 0.9 | 57.31 |
| | 26579 | A_F234K_strat2 | 8.5E−08 | 1.6E−07 | 16.9 | 0.2 | 2.0 | 0.3 | 8.6 | 0.6 | 59.58 |
| | 26580 | A_F234H_strat2 | 1.9E−08 | 5.4E−08 | 75.6 | 0.8 | 5.7 | 0.8 | 13.3 | 1.0 | 50.16 |
| | 26581 | A_F234P_strat2 | 1.2E−08 | 3.0E−08 | 119.7 | 1.3 | 10.2 | 1.5 | 11.7 | 0.9 | 43.43 |
| | 26582 | A_L235G_strat2 | 3.3E−08 | 8.8E−08 | 44.2 | 0.5 | 3.5 | 0.5 | 12.5 | 0.9 | |
| | 26583 | A_L235A_strat2 | 2.7E−08 | 8.1E−08 | 54.2 | 0.6 | 3.8 | 0.5 | 14.3 | 1.1 | 46.65 |
| | 26584 | A_L235V_strat2 | 2.4E−08 | 7.4E−08 | 59.6 | 0.6 | 4.2 | 0.6 | 14.4 | 1.1 | 43.04 |
| | 26585 | A_L235I_strat2 | 2.1E−08 | 5.9E−08 | 69.0 | 0.7 | 5.2 | 0.8 | 13.2 | 1.0 | 40.68 |
| | 26586 | A_L235F_strat2 | 1.5E−08 | 4.6E−08 | 97.3 | 1.0 | 6.7 | 1.0 | 14.4 | 1.1 | 51.23 |
| | 26587 | A_L235W_strat2 | 1.6E−08 | 4.5E−08 | 90.4 | 1.0 | 6.8 | 1.0 | 13.3 | 1.0 | 52.82 |
| | 26588 | A_L235Y_strat2 | 1.2E−08 | 4.3E−08 | 116.9 | 1.3 | 7.1 | 1.0 | 16.4 | 1.2 | 58.15 |
| | 26589 | A_L235T_strat2 | 3.1E−08 | 1.0E−07 | 47.0 | 0.5 | 3.1 | 0.4 | 15.3 | 1.1 | 55.30 |
| | 26590 | A_L235S_strat2 | 3.0E−08 | 9.3E−08 | 47.6 | 0.5 | 3.3 | 0.5 | 14.4 | 1.1 | 55.08 |
| | 26591 | A_L235Q_strat2 | 3.0E−08 | 7.8E−08 | 47.5 | 0.5 | 4.0 | 0.6 | 12.0 | 0.9 | 56.29 |
| | 26592 | A_L235N_strat2 | 4.1E−08 | 1.2E−07 | 34.9 | 0.4 | 2.5 | 0.4 | 14.0 | 1.0 | 69.71 |
| | 26593 | A_L235D_strat2 | 2.2E−08 | 1.2E−07 | 65.2 | 0.7 | 2.5 | 0.4 | 26.3 | 2.0 | 61.21 |
| | 26594 | A_L235E_strat2 | 2.7E−08 | 7.9E−08 | 52.9 | 0.6 | 3.9 | 0.6 | 13.6 | 1.0 | 57.05 |
| | 26595 | A_L235R_strat2 | 9.4E−08 | 3.6E−07 | 15.4 | 0.2 | 0.8 | 0.1 | 18.1 | 1.4 | 59.67 |
| | 26596 | A_L235K_strat2 | ND5 | ND | — | — | — | — | — | — | 60.88 |
| | 26597 | A_L235H_strat2 | 1.8E−08 | 5.9E−08 | 78.4 | 0.8 | 5.2 | 0.8 | 15.0 | 1.1 | 48.99 |
| | 26598 | A_L235P_strat2 | 2.6E−08 | 7.3E−08 | 56.3 | 0.6 | 4.2 | 0.6 | 13.4 | 1.0 | 59.10 |
| | 26599 | A_N236G_strat2 | 7.3E−09 | 2.1E−08 | 198.4 | 2.1 | 14.8 | 2.1 | 13.4 | 1.0 | 41.59 |
| | 26600 | A_N236A_strat2 | 6.4E−09 | 7.1E−09 | 224.6 | 2.4 | 43.2 | 6.2 | 5.2 | 0.4 | 9.17 |
| | 26601 | A_N236V_strat2 | 2.5E−08 | 3.4E−08 | 57.5 | 0.6 | 9.2 | 1.3 | 6.3 | 0.5 | 8.29 |
| | 26602 | A_N236L_strat2 | 3.3E−08 | 5.3E−08 | 43.4 | 0.5 | 5.9 | 0.8 | 7.4 | 0.6 | |
| | 26603 | A_N236I_strat2 | 2.5E−08 | 3.3E−08 | 57.0 | 0.6 | 9.3 | 1.3 | 6.1 | 0.5 | 13.27 |
| | 26604 | A_N236F_strat2 | 8.4E−09 | 2.7E−08 | 171.2 | 1.8 | 11.6 | 1.7 | 14.7 | 1.1 | 57.42 |
| | 26605 | A_N236W_strat2 | 6.4E−09 | 2.0E−08 | 227.1 | 2.4 | 15.2 | 2.2 | 15.0 | 1.1 | 59.65 |
| | 26606 | A_N236Y_strat2 | 7.4E−09 | 2.6E−08 | 195.1 | 2.1 | 11.7 | 1.7 | 16.7 | 1.2 | 63.17 |
| | 26607 | A_N236T_strat2 | 2.8E−08 | 2.8E−08 | 52.4 | 0.6 | 11.2 | 1.6 | 4.7 | 0.3 | 9.17 |
| | 26608 | A_N236S_strat2 | 1.1E−08 | 1.5E−08 | 128.9 | 1.4 | 20.4 | 2.9 | 6.3 | 0.5 | 14.80 |
| | 26609 | A_N236Q_strat2 | 3.0E−08 | 4.3E−08 | 47.8 | 0.5 | 7.1 | 1.0 | 6.7 | 0.5 | 12.14 |
| | 26610 | A_N236D_strat2 | 2.3E−08 | 5.5E−08 | 62.7 | 0.7 | 5.6 | 0.8 | 11.1 | 0.8 | 48.99 |
| | 26611 | A_N236E_strat2 | 3.5E−08 | 4.1E−08 | 41.2 | 0.4 | 7.5 | 1.1 | 5.5 | 0.4 | 12.16 |
| | 26612 | A_N236R_strat2 | ND | ND | — | — | — | — | — | — | 28.40 |
| | 26613 | A_N236K_strat2 | 3.0E−07 | 1.4E−07 | 4.8 | 0.1 | 2.2 | 0.3 | 2.2 | 0.2 | 13.09 |
| | 26614 | A_N236H_strat2 | 2.5E−08 | 2.2E−08 | 56.9 | 0.6 | 13.7 | 2.0 | 4.1 | 0.3 | |
| | 26615 | A_N236P_strat2 | 1.3E−08 | 1.5E−08 | 111.2 | 1.2 | 20.4 | 2.9 | 5.4 | 0.4 | |
| | 26616 | A_G237A_strat2 | 2.5E−08 | 6.6E−08 | 58.6 | 0.6 | 4.7 | 0.7 | 12.6 | 0.9 | 43.06 |
| | 26617 | A_G237V_strat2 | 3.9E−08 | 3.6E−08 | 37.1 | 0.4 | 8.6 | 1.2 | 4.3 | 0.3 | 3.10 |
| | 26618 | A_G237L_strat2 | 1.3E−08 | 5.1E−08 | 115.6 | 1.2 | 6.1 | 0.9 | 19.1 | 1.4 | |
| | 26619 | A_G237I_strat2 | 1.9E−08 | 2.2E−08 | 76.1 | 0.8 | 13.9 | 2.0 | 5.5 | 0.4 | 7.49 |
| | 26620 | A_G237F_strat2 | 3.3E−09 | 7.7E−09 | 442.1 | 4.8 | 39.9 | 5.7 | 11.1 | 0.8 | 33.04 |
| | 26621 | A_G237W_strat2 | 7.5E−09 | 1.7E−08 | 193.3 | 2.1 | 17.7 | 2.6 | 10.9 | 0.8 | 52.67 |
| | 26622 | A_G237Y_strat2 | 8.9E−09 | 2.9E−08 | 162.2 | 1.7 | 10.6 | 1.5 | 15.2 | 1.1 | 62.57 |
| | 26623 | A_G237T_strat2 | 3.0E−08 | 7.2E−08 | 47.9 | 0.5 | 4.3 | 0.6 | 11.1 | 0.8 | 43.93 |
| | 26624 | A_G237S_strat2 | 3.7E−08 | 9.6E−08 | 39.5 | 0.4 | 3.2 | 0.5 | 12.3 | 0.9 | 43.86 |
| | 26625 | A_G237Q_strat2 | 9.5E−08 | 4.8E−08 | 15.2 | 0.2 | 6.4 | 0.9 | 2.4 | 0.2 | 54.77 |
| | 26626 | A_G237N_strat2 | 3.0E−08 | 9.2E−08 | 48.3 | 0.5 | 3.3 | 0.5 | 14.5 | 1.1 | 61.33 |
| | 26627 | A_G237D_strat2 | 5.1E−08 | 1.8E−07 | 28.3 | 0.3 | 1.7 | 0.2 | 16.4 | 1.2 | 64.03 |
| | 26628 | A_G237E_strat2 | 1.1E−07 | 2.0E−07 | 13.1 | 0.1 | 1.5 | 0.2 | 8.7 | 0.6 | 46.81 |
| | 26629 | A_G237R_strat2 | ND | ND | — | — | — | — | — | — | 80.75 |
| | 26630 | A_G237K_strat2 | ND | ND | — | — | — | — | — | — | 43.07 |
| | 26631 | A_G237H_strat2 | 3.5E−08 | 6.8E−08 | 41.7 | 0.4 | 4.5 | 0.7 | 9.2 | 0.7 | 40.21 |
| | 26632 | A_G237P_strat2 | 1.9E−08 | 2.1E−08 | 74.3 | 0.8 | 14.5 | 2.1 | 5.1 | 0.4 | 6.98 |
| | 26633 | A_S239G_strat2 | 1.8E−08 | 7.1E−08 | 81.7 | 0.9 | 4.3 | 0.6 | 18.9 | 1.4 | 56.44 |
| | 26634 | A_S239A_strat2 | 2.3E−08 | 6.7E−08 | 62.6 | 0.7 | 4.6 | 0.7 | 13.6 | 1.0 | 48.03 |
| | 26635 | A_S239V_strat2 | 1.9E−08 | 5.9E−08 | 74.7 | 0.8 | 5.3 | 0.8 | 14.2 | 1.1 | 48.56 |
| | 26636 | A_S239L_strat2 | 1.7E−08 | 5.9E−08 | 86.0 | 0.9 | 5.3 | 0.8 | 16.4 | 1.2 | 65.59 |
| | 26637 | A_S239I_strat2 | 2.8E−08 | 5.9E−08 | 51.7 | 0.6 | 5.3 | 0.8 | 9.8 | 0.7 | 63.92 |
| | 26638 | A_S239F_strat2 | 5.2E−08 | 1.4E−07 | 28.0 | 0.3 | 2.1 | 0.3 | 13.1 | 1.0 | 73.55 |
| | 26639 | A_S239W_strat2 | 8.5E−08 | 1.6E−07 | 17.0 | 0.2 | 1.9 | 0.3 | 9.0 | 0.7 | 50.97 |
| | 26640 | A_S239Y_strat2 | 5.1E−08 | 1.2E−07 | 28.1 | 0.3 | 2.6 | 0.4 | 10.7 | 0.8 | 60.05 |
| | 26641 | A_S239T_strat2 | 3.9E−08 | 2.1E−08 | 36.9 | 0.4 | 15.0 | 2.2 | 2.5 | 0.2 | 51.16 |
| | 26642 | A_S239Q_strat2 | 2.3E−08 | 7.0E−08 | 62.9 | 0.7 | 4.4 | 0.6 | 14.3 | 1.1 | 56.33 |
| | 26643 | A_S239N_strat2 | 2.1E−08 | 7.2E−08 | 69.2 | 0.7 | 4.3 | 0.6 | 16.1 | 1.2 | 52.70 |
| | 26644 | A_S239D_strat2 | 1.9E−08 | 5.9E−08 | 78.0 | 0.8 | 5.2 | 0.8 | 14.9 | 1.1 | 46.41 |
| | 26645 | A_S239E_strat2 | 2.1E−08 | 6.2E−08 | 70.5 | 0.8 | 5.0 | 0.7 | 14.1 | 1.1 | 48.49 |
| | 26646 | A_S239R_strat2 | 2.6E−08 | 4.8E−08 | 56.1 | 0.6 | 6.4 | 0.9 | 8.7 | 0.7 | 34.64 |
| | 26647 | A_S239K_strat2 | 3.5E−08 | 9.4E−08 | 40.8 | 0.4 | 3.3 | 0.5 | 12.4 | 0.9 | 45.51 |
| | 26648 | A_S239H_strat2 | 3.6E−08 | 1.0E−07 | 40.1 | 0.4 | 3.1 | 0.4 | 13.0 | 1.0 | 59.34 |
| | 26649 | A_S239P_strat2 | ND | ND | — | — | — | — | — | — | 68.20 |
| | 26650 | A_V264A_strat2 | 3.0E−08 | 6.8E−08 | 48.4 | 0.5 | 4.5 | 0.7 | 10.7 | 0.8 | 44.38 |
| | 26651 | A_V264L_strat2 | 2.1E−08 | 5.4E−08 | 70.3 | 0.8 | 5.7 | 0.8 | 12.4 | 0.9 | 39.58 |
| | 26652 | A_V264I_strat2 | 1.7E−08 | 4.5E−08 | 83.2 | 0.9 | 6.8 | 1.0 | 12.1 | 0.9 | 48.16 |
| | 26653 | A_V264M_strat2 | 2.0E−08 | 5.3E−08 | 73.3 | 0.8 | 5.8 | 0.8 | 12.6 | 0.9 | 56.83 |
| | 26654 | A_V264F_strat2 | 1.5E−08 | 4.9E−08 | 94.3 | 1.0 | 6.2 | 0.9 | 15.1 | 1.1 | 61.18 |
| | 26655 | A_V264T_strat2 | 2.0E−08 | 4.4E−08 | 71.2 | 0.8 | 7.0 | 1.0 | 10.1 | 0.8 | 56.17 |
| | 26656 | A_V266A_strat2 | 4.3E−08 | 9.2E−08 | 33.5 | 0.4 | 3.3 | 0.5 | 10.0 | 0.7 | 63.23 |
| | 26657 | A_V266L_strat2 | 3.2E−08 | 9.2E−08 | 45.2 | 0.5 | 3.3 | 0.5 | 13.5 | 1.0 | 58.50 |
| | 26658 | A_V266I_strat2 | 1.6E−08 | 5.2E−08 | 88.8 | 1.0 | 6.0 | 0.9 | 14.9 | 1.1 | 49.25 |
| | 26659 | A_V266M_strat2 | 5.1E−08 | 1.5E−07 | 28.5 | 0.3 | 2.0 | 0.3 | 14.0 | 1.0 | 56.62 |
| | 26660 | A_V266F_strat2 | 7.4E−08 | 2.1E−07 | 19.6 | 0.2 | 1.5 | 0.2 | 13.1 | 1.0 | 66.36 |
| | 26661 | A_V266T_strat2 | ND | ND | — | — | — | — | — | — | 69.13 |
| | 26662 | A_S267G_strat2 | 2.9E−08 | 9.2E−08 | 49.1 | 0.5 | 3.3 | 0.5 | 14.7 | 1.1 | 52.74 |
| | 26663 | A_S267A_strat2 | 2.3E−08 | 1.2E−07 | 62.1 | 0.7 | 2.6 | 0.4 | 23.6 | 1.8 | 53.39 |
| | 26664 | A_S267V_strat2 | 2.8E−08 | 7.2E−08 | 52.5 | 0.6 | 4.3 | 0.6 | 12.2 | 0.9 | 43.86 |
| | 26665 | A_S267L_strat2 | 3.7E−08 | 8.0E−08 | 39.4 | 0.4 | 3.8 | 0.6 | 10.3 | 0.8 | 41.49 |
| | 26666 | A_S267I_strat2 | 2.1E−08 | 5.7E−08 | 68.0 | 0.7 | 5.4 | 0.8 | 12.6 | 0.9 | 41.37 |
| | 26667 | A_S267F_strat2 | 3.5E−08 | 1.1E−07 | 41.5 | 0.4 | 2.8 | 0.4 | 14.8 | 1.1 | 43.68 |
| | 26668 | A_S267W_strat2 | 3.2E−08 | 9.1E−08 | 45.0 | 0.5 | 3.4 | 0.5 | 13.2 | 1.0 | 59.49 |
| | 26669 | A_S267Y_strat2 | 4.8E−08 | 1.6E−07 | 29.9 | 0.3 | 2.0 | 0.3 | 15.3 | 1.1 | 66.20 |
| | 26670 | A_S267T_strat2 | 2.0E−08 | 5.2E−08 | 71.2 | 0.8 | 6.0 | 0.9 | 11.9 | 0.9 | 53.93 |
| | 26671 | A_S267Q_strat2 | 4.4E−08 | 1.4E−07 | 32.8 | 0.4 | 2.2 | 0.3 | 15.0 | 1.1 | 56.18 |
| | 26672 | A_S267N_strat2 | 2.8E−08 | 8.2E−08 | 51.6 | 0.6 | 3.8 | 0.5 | 13.7 | 1.0 | 59.95 |
| | 26673 | A_S267R_strat2 | 7.2E−08 | 2.0E−07 | 20.0 | 0.2 | 1.5 | 0.2 | 13.0 | 1.0 | 57.91 |
| | 26674 | A_S267K_strat2 | 6.4E−08 | 2.4E−07 | 22.7 | 0.2 | 1.3 | 0.2 | 17.7 | 1.3 | 56.75 |
| | 26675 | A_S267H_strat2 | 2.8E−08 | 1.0E−07 | 51.2 | 0.6 | 3.0 | 0.4 | 17.3 | 1.3 | 64.34 |
| | 26676 | A_S267P_strat2 | 2.5E−08 | 7.9E−08 | 58.3 | 0.6 | 3.9 | 0.6 | 15.0 | 1.1 | |
| | 26677 | A_Q268G_strat2 | 2.1E−08 | 7.1E−08 | 69.9 | 0.8 | 4.4 | 0.6 | 16.0 | 1.2 | 47.50 |
| | 26678 | A_Q268A_strat2 | 2.2E−08 | 6.4E−08 | 64.4 | 0.7 | 4.8 | 0.7 | 13.3 | 1.0 | 44.03 |
| | 26679 | A_Q268V_strat2 | 2.2E−08 | 4.7E−08 | 66.6 | 0.7 | 6.6 | 0.9 | 10.1 | 0.8 | 43.83 |
| | 26680 | A_Q268L_strat2 | 2.2E−08 | 6.2E−08 | 64.8 | 0.7 | 5.0 | 0.7 | 13.0 | 1.0 | 47.63 |
| | 26681 | A_Q268I_strat2 | 2.4E−08 | 7.2E−08 | 60.8 | 0.7 | 4.3 | 0.6 | 14.1 | 1.1 | 43.40 |
| | 26682 | A_Q268F_strat2 | 1.7E−08 | 3.9E−08 | 86.0 | 0.9 | 8.0 | 1.1 | 10.8 | 0.8 | 42.88 |
| | 26683 | A_Q268W_strat2 | 1.6E−08 | 4.9E−08 | 92.2 | 1.0 | 6.3 | 0.9 | 14.6 | 1.1 | 42.41 |
| | 26684 | A_Q268Y_strat2 | 1.6E−08 | 4.7E−08 | 89.6 | 1.0 | 6.6 | 0.9 | 13.7 | 1.0 | 36.00 |
| | 26685 | A_Q268T_strat2 | 1.5E−08 | 5.2E−08 | 94.1 | 1.0 | 5.9 | 0.9 | 15.9 | 1.2 | |
| | 26686 | A_Q268S_strat2 | 2.2E−08 | 6.3E−08 | 65.7 | 0.7 | 4.9 | 0.7 | 13.3 | 1.0 | 47.91 |
| | 26687 | A_Q268N_strat2 | 2.0E−08 | 5.4E−08 | 71.5 | 0.8 | 5.7 | 0.8 | 12.5 | 0.9 | 49.60 |
| | 26688 | A_Q268D_strat2 | 2.0E−08 | 5.8E−08 | 71.6 | 0.8 | 5.3 | 0.8 | 13.5 | 1.0 | 47.94 |
| | 26689 | A_Q268E_strat2 | 2.6E−08 | 7.3E−08 | 55.1 | 0.6 | 4.2 | 0.6 | 13.0 | 1.0 | 48.77 |
| | 26690 | A_Q268R_strat2 | 1.8E−08 | 5.8E−08 | 79.4 | 0.9 | 5.3 | 0.8 | 14.9 | 1.1 | 51.13 |
| | 26691 | A_Q268K_strat2 | 1.7E−08 | 5.5E−08 | 84.5 | 0.9 | 5.6 | 0.8 | 15.1 | 1.1 | 43.52 |
| | 26692 | A_Q268H_strat2 | 1.5E−08 | 4.6E−08 | 94.0 | 1.0 | 6.7 | 1.0 | 14.0 | 1.0 | 45.20 |
| | 26693 | A_Q268P_strat2 | 1.5E−08 | 5.2E−08 | 96.3 | 1.0 | 6.0 | 0.9 | 16.1 | 1.2 | 40.47 |
| | 26694 | A_E269G_strat2 | 3.0E−08 | 7.8E−08 | 48.3 | 0.5 | 4.0 | 0.6 | 12.1 | 0.9 | 46.78 |
| | 26695 | A_E269A_strat2 | 2.4E−08 | 6.8E−08 | 59.8 | 0.6 | 4.5 | 0.7 | 13.2 | 1.0 | 41.75 |
| | 26696 | A_E269V_strat2 | 2.2E−08 | 6.1E−08 | 66.8 | 0.7 | 5.0 | 0.7 | 13.3 | 1.0 | 43.86 |
| | 26697 | A_E269L_strat2 | 2.0E−08 | 6.0E−08 | 74.0 | 0.8 | 5.1 | 0.7 | 14.5 | 1.1 | 45.41 |
| | 26698 | A_E269I_strat2 | 2.0E−08 | 6.1E−08 | 71.8 | 0.8 | 5.1 | 0.7 | 14.1 | 1.1 | 43.49 |
| | 26699 | A_E269F_strat2 | 2.2E−08 | 5.1E−08 | 65.7 | 0.7 | 6.0 | 0.9 | 11.0 | 0.8 | 56.34 |
| | 26700 | A_E269W_strat2 | 1.7E−08 | 6.6E−08 | 83.1 | 0.9 | 4.6 | 0.7 | 17.9 | 1.3 | 42.94 |
| | 26701 | A_E269Y_strat2 | 2.1E−08 | 6.7E−08 | 67.2 | 0.7 | 4.6 | 0.7 | 14.7 | 1.1 | 53.14 |
| | 26702 | A_E269T_strat2 | 2.6E−08 | 6.4E−08 | 56.0 | 0.6 | 4.8 | 0.7 | 11.6 | 0.9 | 49.76 |
| | 26703 | A_E269S_strat2 | 2.4E−08 | 6.0E−08 | 60.4 | 0.6 | 5.1 | 0.7 | 11.8 | 0.9 | 49.53 |
| | 26704 | A_E269Q_strat2 | 2.2E−08 | 6.6E−08 | 67.2 | 0.7 | 4.7 | 0.7 | 14.3 | 1.1 | 54.09 |
| | 26705 | A_E269N_strat2 | 2.6E−08 | 8.3E−08 | 54.7 | 0.6 | 3.7 | 0.5 | 14.8 | 1.1 | 49.14 |
| | 26706 | A_E269D_strat2 | 2.0E−08 | 6.1E−08 | 73.6 | 0.8 | 5.0 | 0.7 | 14.7 | 1.1 | 50.57 |
| | 26707 | A_E269R_strat2 | 2.3E−08 | 7.7E−08 | 63.2 | 0.7 | 4.0 | 0.6 | 15.8 | 1.2 | 57.28 |
| | 26708 | A_E269K_strat2 | 2.2E−08 | 7.0E−08 | 65.8 | 0.7 | 4.4 | 0.6 | 15.0 | 1.1 | 51.03 |
| | 26709 | A_E269H_strat2 | 2.2E−08 | 6.9E−08 | 64.5 | 0.7 | 4.5 | 0.6 | 14.4 | 1.1 | 47.89 |
| | 26710 | A_E269P_strat2 | 2.7E−08 | 7.8E−08 | 52.8 | 0.6 | 3.9 | 0.6 | 13.4 | 1.0 | 45.81 |
| | 26711 | A_D270G_strat2 | 5.3E−08 | 1.8E−07 | 27.5 | 0.3 | 1.7 | 0.2 | 16.0 | 1.2 | 48.60 |
| | 26712 | A_D270A_strat2 | 2.9E−08 | 8.0E−08 | 49.9 | 0.5 | 3.8 | 0.6 | 13.0 | 1.0 | 45.88 |
| | 26713 | A_D270V_strat2 | 4.1E−08 | 8.9E−08 | 35.4 | 0.4 | 3.5 | 0.5 | 10.2 | 0.8 | 42.68 |
| | 26714 | A_D270L_strat2 | 3.3E−08 | 7.9E−08 | 43.4 | 0.5 | 3.9 | 0.6 | 11.1 | 0.8 | 43.35 |
| | 26715 | A_D270I_strat2 | 2.5E−08 | 8.1E−08 | 57.9 | 0.6 | 3.8 | 0.5 | 15.2 | 1.1 | 40.12 |
| | 26716 | A_D270F_strat2 | 1.7E−08 | 4.8E−08 | 86.7 | 0.9 | 6.4 | 0.9 | 13.5 | 1.0 | 46.83 |
| | 26717 | A_D270W_strat2 | 2.7E−08 | 8.3E−08 | 53.5 | 0.6 | 3.7 | 0.5 | 14.5 | 1.1 | 45.39 |
| | 26718 | A_D270Y_strat2 | 2.6E−08 | 6.9E−08 | 54.6 | 0.6 | 4.5 | 0.6 | 12.1 | 0.9 | 52.44 |
| | 26719 | A_D270T_strat2 | 2.3E−08 | 6.3E−08 | 64.2 | 0.7 | 4.9 | 0.7 | 13.2 | 1.0 | 54.53 |
| | 26720 | A_D270S_strat2 | 2.9E−08 | 7.9E−08 | 49.5 | 0.5 | 3.9 | 0.6 | 12.7 | 0.9 | 55.16 |
| | 26721 | A_D270Q_strat2 | 2.8E−08 | 8.7E−08 | 51.7 | 0.6 | 3.6 | 0.5 | 14.6 | 1.1 | 53.35 |
| | 26722 | A_D270N_strat2 | 2.1E−08 | 7.7E−08 | 67.4 | 0.7 | 4.0 | 0.6 | 16.7 | 1.3 | 62.02 |
| | 26723 | A_D270E_strat2 | 1.3E−08 | 4.2E−08 | 109.0 | 1.2 | 7.3 | 1.0 | 15.0 | 1.1 | 49.90 |
| | 26724 | A_D270R_strat2 | 8.9E−08 | 1.4E−07 | 16.2 | 0.2 | 2.1 | 0.3 | 7.6 | 0.6 | 66.39 |
| | 26725 | A_D270K_strat2 | 6.0E−08 | 1.6E−07 | 24.1 | 0.3 | 1.9 | 0.3 | 12.5 | 0.9 | |
| | 26726 | A_D270H_strat2 | 2.3E−08 | 7.4E−08 | 61.7 | 0.7 | 4.1 | 0.6 | 14.9 | 1.1 | 52.16 |
| | 26727 | A_D270P_strat2 | 8.8E−08 | 1.8E−07 | 16.4 | 0.2 | 1.7 | 0.2 | 9.4 | 0.7 | 60.54 |
| | 26728 | A_P271G_strat2 | 2.2E−08 | 6.4E−08 | 65.4 | 0.7 | 4.8 | 0.7 | 13.5 | 1.0 | 44.42 |
| | 26729 | A_P271A_strat2 | 3.4E−08 | 9.3E−08 | 42.1 | 0.5 | 3.3 | 0.5 | 12.7 | 1.0 | 46.17 |
| | 26730 | A_P271V_strat2 | 2.7E−08 | 8.5E−08 | 54.4 | 0.6 | 3.6 | 0.5 | 15.0 | 1.1 | 45.61 |
| | 26731 | A_P271L_strat2 | 2.2E−08 | 6.8E−08 | 66.6 | 0.7 | 4.5 | 0.7 | 14.7 | 1.1 | 44.74 |
| | 26732 | A_P271I_strat2 | 2.5E−08 | 7.0E−08 | 56.9 | 0.6 | 4.4 | 0.6 | 12.9 | 1.0 | 42.64 |
| | 26733 | A_P271F_strat2 | 3.1E−08 | 8.7E−08 | 46.3 | 0.5 | 3.6 | 0.5 | 13.0 | 1.0 | 44.95 |
| | 26734 | A_P271W_strat2 | 2.8E−08 | 7.2E−08 | 52.5 | 0.6 | 4.3 | 0.6 | 12.3 | 0.9 | 45.16 |
| | 26735 | A_P271Y_strat2 | 3.5E−08 | 1.1E−07 | 41.9 | 0.5 | 2.8 | 0.4 | 14.8 | 1.1 | 53.12 |
| | 26736 | A_P271T_strat2 | 4.7E−08 | 1.2E−07 | 30.8 | 0.3 | 2.5 | 0.4 | 12.5 | 0.9 | 55.05 |
| | 26737 | A_P271S_strat2 | 3.6E−08 | 9.0E−08 | 40.7 | 0.4 | 3.4 | 0.5 | 11.9 | 0.9 | 52.79 |
| | 26738 | A_P271Q_strat2 | 2.6E−08 | 7.7E−08 | 56.1 | 0.6 | 4.0 | 0.6 | 14.1 | 1.1 | 55.49 |
| | 26739 | A_P271N_strat2 | 2.8E−08 | 9.0E−08 | 50.9 | 0.5 | 3.4 | 0.5 | 14.9 | 1.1 | 61.54 |
| | 26740 | A_P271D_strat2 | 2.9E−08 | 9.1E−08 | 50.5 | 0.5 | 3.4 | 0.5 | 14.9 | 1.1 | 54.68 |
| | 26741 | A_P271E_strat2 | 2.4E−08 | 6.1E−08 | 59.3 | 0.6 | 5.1 | 0.7 | 11.7 | 0.9 | 49.96 |
| | 26742 | A_P271R_strat2 | 3.1E−08 | 7.0E−08 | 47.1 | 0.5 | 4.4 | 0.6 | 10.7 | 0.8 | 46.00 |
| | 26743 | A_P271K_strat2 | 2.6E−08 | 7.1E−08 | 55.7 | 0.6 | 4.3 | 0.6 | 12.9 | 1.0 | 42.19 |
| | 26744 | A_P271H_strat2 | 2.4E−08 | 7.9E−08 | 59.3 | 0.6 | 3.9 | 0.6 | 15.1 | 1.1 | |
| | 26745 | A_E272G_strat2 | 2.8E−08 | 6.9E−08 | 51.6 | 0.6 | 4.5 | 0.6 | 11.6 | 0.9 | 43.38 |
| | 26746 | A_E272A_strat2 | 2.0E−08 | 6.9E−08 | 70.8 | 0.8 | 4.5 | 0.6 | 15.9 | 1.2 | 42.72 |
| | 26747 | A_E272V_strat2 | 1.6E−08 | 5.1E−08 | 89.7 | 1.0 | 6.0 | 0.9 | 14.9 | 1.1 | 39.15 |
| | 26748 | A_E272L_strat2 | 1.8E−08 | 5.8E−08 | 81.3 | 0.9 | 5.3 | 0.8 | 15.2 | 1.1 | 36.52 |
| | 26749 | A_E272I_strat2 | 1.7E−08 | 5.1E−08 | 83.8 | 0.9 | 6.1 | 0.9 | 13.7 | 1.0 | 39.56 |
| | 26750 | A_E272F_strat2 | 2.4E−08 | 6.8E−08 | 61.2 | 0.7 | 4.5 | 0.6 | 13.5 | 1.0 | 44.19 |
| | 26751 | A_E272W_strat2 | 2.4E−08 | 5.7E−08 | 60.1 | 0.6 | 5.4 | 0.8 | 11.2 | 0.8 | 41.65 |
| | 26752 | A_E272Y_strat2 | 1.8E−08 | 6.8E−08 | 79.8 | 0.9 | 4.6 | 0.7 | 17.5 | 1.3 | 48.44 |
| | 26753 | A_E272T_strat2 | 2.3E−08 | 7.2E−08 | 62.8 | 0.7 | 4.3 | 0.6 | 14.7 | 1.1 | 50.41 |
| | 26754 | A_E272S_strat2 | 2.4E−08 | 7.5E−08 | 61.0 | 0.7 | 4.1 | 0.6 | 14.7 | 1.1 | 50.68 |
| | 26755 | A_E272Q_strat2 | 2.5E−08 | 2.0E−08 | 58.5 | 0.6 | 15.1 | 2.2 | 3.9 | 0.3 | 51.50 |
| | 26756 | A_E272N_strat2 | 2.5E−08 | 8.5E−08 | 57.6 | 0.6 | 3.6 | 0.5 | 15.8 | 1.2 | 52.61 |
| | 26757 | A_E272D_strat2 | 2.2E−08 | 6.3E−08 | 66.6 | 0.7 | 4.9 | 0.7 | 13.5 | 1.0 | 45.11 |
| | 26758 | A_E272R_strat2 | 3.4E−08 | 1.0E−07 | 42.6 | 0.5 | 3.1 | 0.4 | 13.8 | 1.0 | 48.41 |
| | 26759 | A_E272K_strat2 | 3.1E−08 | 1.6E−08 | 47.2 | 0.5 | 19.0 | 2.7 | 2.5 | 0.2 | 50.76 |
| | 26760 | A_E272H_strat2 | 2.0E−08 | 6.4E−08 | 74.1 | 0.8 | 4.9 | 0.7 | 15.3 | 1.1 | |
| | 26761 | A_E272P_strat2 | 9.8E−08 | 3.0E−07 | 14.7 | 0.2 | 1.0 | 0.1 | 14.4 | 1.1 | 50.78 |
| | 26762 | A_V273A_strat2 | 2.7E−08 | 9.7E−08 | 52.7 | 0.6 | 3.2 | 0.5 | 16.5 | 1.2 | 44.75 |
| | 26763 | A_V273L_strat2 | 6.1E−08 | 1.7E−07 | 23.9 | 0.3 | 1.8 | 0.3 | 13.2 | 1.0 | 46.18 |
| | 26764 | A_V273I_strat2 | 4.5E−08 | 1.4E−07 | 32.0 | 0.3 | 2.3 | 0.3 | 14.0 | 1.0 | 43.62 |
| | 26765 | A_V273M_strat2 | 1.1E−07 | 3.2E−07 | 13.6 | 0.1 | 1.0 | 0.1 | 13.9 | 1.0 | 53.60 |
| | 26766 | A_V273F_strat2 | 1.1E−07 | 3.6E−07 | 13.4 | 0.1 | 0.9 | 0.1 | 15.6 | 1.2 | 57.65 |
| | 26767 | A_V273T_strat2 | 3.4E−08 | 9.6E−08 | 42.3 | 0.5 | 3.2 | 0.5 | 13.2 | 1.0 | 43.55 |
| | 26768 | A_V323A_strat2 | 3.0E−08 | 9.5E−08 | 47.5 | 0.5 | 3.2 | 0.5 | 14.7 | 1.1 | 60.62 |
| | 26769 | A_V323L_strat2 | 1.7E−08 | 4.9E−08 | 83.5 | 0.9 | 6.4 | 0.9 | 13.1 | 1.0 | 56.07 |
| | 26770 | A_V323I_strat2 | 1.8E−08 | 5.2E−08 | 81.2 | 0.9 | 5.9 | 0.8 | 13.8 | 1.0 | 48.23 |
| | 26771 | A_V323M_strat2 | 4.0E−08 | 1.2E−07 | 35.9 | 0.4 | 2.6 | 0.4 | 13.7 | 1.0 | 60.31 |
| | 26772 | A_V323F_strat2 | 6.1E−08 | 1.8E−07 | 23.6 | 0.3 | 1.8 | 0.3 | 13.4 | 1.0 | 57.36 |
| | 26773 | A_V323T_strat2 | 3.8E−08 | 1.0E−07 | 37.9 | 0.4 | 3.0 | 0.4 | 12.5 | 0.9 | |
| | 26774 | A_N325G_strat2 | 3.4E−07 | 5.7E−07 | 4.3 | 0.0 | 0.5 | 0.1 | 8.0 | 0.6 | 94.68 |
| | 26775 | A_N325A_strat2 | 7.8E−08 | 2.2E−07 | 18.5 | 0.2 | 1.4 | 0.2 | 13.3 | 1.0 | 61.98 |
| | 26776 | A_N325V_strat2 | 1.0E−07 | 3.1E−07 | 13.9 | 0.1 | 1.0 | 0.1 | 14.0 | 1.0 | 69.86 |
| | 26777 | A_N325L_strat2 | 1.3E−07 | 4.2E−07 | 11.2 | 0.1 | 0.7 | 0.1 | 15.1 | 1.1 | 62.43 |
| | 26778 | A_N325I_strat2 | 8.4E−08 | 2.9E−07 | 17.1 | 0.2 | 1.1 | 0.2 | 16.1 | 1.2 | 57.49 |
| | 26779 | A_N325F_strat2 | ND | ND | — | — | — | — | — | — | |
| | 26780 | A_N325W_strat2 | ND | ND | — | — | — | — | — | — | 67.80 |
| | 26781 | A_N325Y_strat2 | 1.7E−07 | 4.6E−07 | 8.7 | 0.1 | 0.7 | 0.1 | 13.1 | 1.0 | −291.55 |
| | 26782 | A_N325T_strat2 | 1.3E−07 | 3.8E−07 | 11.6 | 0.1 | 0.8 | 0.1 | 14.1 | 1.1 | 69.02 |
| | 26783 | A_N325S_strat2 | 9.5E−08 | 3.2E−07 | 15.2 | 0.2 | 1.0 | 0.1 | 15.7 | 1.2 | 66.91 |
| | 26784 | A_N325Q_strat2 | 8.4E−08 | 2.4E−07 | 17.3 | 0.2 | 1.3 | 0.2 | 13.2 | 1.0 | 65.07 |
| | 26785 | A_N325D_strat2 | 7.5E−08 | 2.5E−07 | 19.2 | 0.2 | 1.2 | 0.2 | 15.7 | 1.2 | 71.24 |
| | 26786 | A_N325E_strat2 | 1.4E−07 | 3.6E−07 | 10.5 | 0.1 | 0.8 | 0.1 | 12.4 | 0.9 | 63.87 |
| | 26787 | A_N325R_strat2 | 2.6E−07 | 5.9E−07 | 5.7 | 0.1 | 0.5 | 0.1 | 10.9 | 0.8 | 78.73 |
| | 26788 | A_N325K_strat2 | 1.7E−07 | 5.6E−07 | 8.7 | 0.1 | 0.6 | 0.1 | 15.7 | 1.2 | 77.84 |
| | 26789 | A_N325H_strat2 | 7.4E−08 | 2.0E−07 | 19.7 | 0.2 | 1.6 | 0.2 | 12.6 | 0.9 | 61.26 |
| | 26790 | A_N325P_strat2 | 2.8E−07 | 8.1E−07 | 5.2 | 0.1 | 0.4 | 0.1 | 13.6 | 1.0 | 84.64 |
| | 26791 | A_K326G_strat2 | ND | ND | — | — | — | — | — | — | |
| | 26792 | A_K326A_strat2 | 2.2E−08 | 6.6E−08 | 66.3 | 0.7 | 4.7 | 0.7 | 14.1 | 1.1 | 50.84 |
| | 26793 | A_K326V_strat2 | 6.3E−08 | 1.9E−07 | 22.9 | 0.2 | 1.7 | 0.2 | 13.7 | 1.0 | 64.22 |
| | 26794 | A_K326L_strat2 | 6.0E−08 | 1.8E−07 | 24.2 | 0.3 | 1.7 | 0.2 | 14.1 | 1.1 | 49.22 |
| | 26795 | A_K326I_strat2 | 1.0E−07 | 2.9E−07 | 14.5 | 0.2 | 1.1 | 0.2 | 13.7 | 1.0 | 60.37 |
| | 26796 | A_K326F_strat2 | 1.0E−07 | 2.6E−07 | 14.2 | 0.2 | 1.2 | 0.2 | 11.9 | 0.9 | |
| | 26797 | A_K326W_strat2 | 6.5E−08 | 1.8E−07 | 22.2 | 0.2 | 1.7 | 0.2 | 13.1 | 1.0 | 51.77 |
| | 26798 | A_K326Y_strat2 | 5.6E−08 | 1.6E−07 | 25.9 | 0.3 | 2.0 | 0.3 | 13.0 | 1.0 | 50.91 |
| | 26799 | A_K326T_strat2 | 2.4E−08 | 7.1E−08 | 59.5 | 0.6 | 4.4 | 0.6 | 13.6 | 1.0 | 46.08 |
| | 26800 | A_K326S_strat2 | 1.8E−08 | 5.3E−08 | 82.6 | 0.9 | 5.9 | 0.8 | 14.1 | 1.1 | 43.69 |
| | 26801 | A_K326Q_strat2 | 2.6E−08 | 7.5E−08 | 55.8 | 0.6 | 4.1 | 0.6 | 13.5 | 1.0 | 47.87 |
| | 26802 | A_K326N_strat2 | 1.6E−08 | 4.9E−08 | 88.7 | 1.0 | 6.3 | 0.9 | 14.2 | 1.1 | 48.59 |
| | 26803 | A_K326D_strat2 | 2.2E−08 | 6.4E−08 | 65.4 | 0.7 | 4.8 | 0.7 | 13.7 | 1.0 | 51.13 |
| | 26804 | A_K326E_strat2 | 3.7E−08 | 1.1E−07 | 38.8 | 0.4 | 2.7 | 0.4 | 14.4 | 1.1 | 52.70 |
| | 26805 | A_K326R_strat2 | 2.1E−08 | 6.1E−08 | 69.5 | 0.7 | 5.1 | 0.7 | 13.7 | 1.0 | 52.14 |
| | 26806 | A_K326H_strat2 | 3.0E−08 | 8.9E−08 | 48.0 | 0.5 | 3.5 | 0.5 | 13.8 | 1.0 | 53.46 |
| | 26807 | A_K326P_strat2 | 3.2E−08 | 9.2E−08 | 45.3 | 0.5 | 3.4 | 0.5 | 13.5 | 1.0 | 51.82 |
| | 26808 | A_G327A_strat2 | 2.4E−08 | 7.0E−08 | 60.7 | 0.7 | 4.4 | 0.6 | 13.8 | 1.0 | 45.66 |
| | 26809 | A_G327V_strat2 | 9.9E−08 | 2.9E−07 | 14.5 | 0.2 | 1.1 | 0.2 | 13.6 | 1.0 | 53.46 |
| | 26810 | A_G327L_strat2 | 5.9E−08 | 1.9E−07 | 24.6 | 0.3 | 1.7 | 0.2 | 14.8 | 1.1 | 56.51 |
| | 26811 | A_G327I_strat2 | 1.4E−07 | 4.3E−07 | 10.2 | 0.1 | 0.7 | 0.1 | 14.1 | 1.1 | 54.99 |
| | 26812 | A_G327F_strat2 | 1.1E−07 | 3.6E−07 | 13.1 | 0.1 | 0.9 | 0.1 | 15.3 | 1.1 | 67.45 |
| | 26813 | A_G327W_strat2 | 1.1E−07 | 3.3E−07 | 12.9 | 0.1 | 0.9 | 0.1 | 13.7 | 1.0 | 69.05 |
| | 26814 | A_G327Y_strat2 | 1.2E−07 | 3.6E−07 | 12.2 | 0.1 | 0.9 | 0.1 | 14.3 | 1.1 | 59.86 |
| | 26815 | A_G327Q_strat2 | 3.6E−08 | 1.0E−07 | 40.7 | 0.4 | 3.0 | 0.4 | 13.5 | 1.0 | 45.05 |
| | 26816 | A_G327N_strat2 | 4.8E−08 | 1.7E−07 | 30.0 | 0.3 | 1.9 | 0.3 | 16.0 | 1.2 | 46.35 |
| | 26817 | A_G327D_strat2 | 3.9E−08 | 1.2E−07 | 36.8 | 0.4 | 2.6 | 0.4 | 14.3 | 1.1 | 43.32 |
| | 26818 | A_G327E_strat2 | 2.5E−08 | 8.1E−08 | 58.5 | 0.6 | 3.8 | 0.5 | 15.4 | 1.2 | 54.61 |
| | 26819 | A_G327R_strat2 | 1.2E−07 | 3.5E−07 | 11.7 | 0.1 | 0.9 | 0.1 | 13.2 | 1.0 | 69.45 |
| | 26820 | A_G327K_strat2 | 1.2E−07 | 3.7E−07 | 11.7 | 0.1 | 0.8 | 0.1 | 14.2 | 1.1 | 63.92 |
| | 26821 | A_G327H_strat2 | 7.8E−08 | 2.2E−07 | 18.6 | 0.2 | 1.4 | 0.2 | 13.3 | 1.0 | 68.53 |
| | 26822 | A_G327P_strat2 | 1.4E−07 | 4.0E−07 | 10.1 | 0.1 | 0.8 | 0.1 | 13.2 | 1.0 | 60.08 |
| | 26823 | A_P329G_strat2 | ND | ND | — | — | — | — | — | — | 123.03 |
| | 26824 | A_P329A_strat2 | 5.3E−08 | 2.2E−07 | 27.5 | 0.3 | 1.4 | 0.2 | 19.5 | 1.5 | 74.06 |
| | 26825 | A_P329V_strat2 | 5.4E−08 | 2.1E−07 | 26.7 | 0.3 | 1.5 | 0.2 | 17.8 | 1.3 | 74.37 |
| | 26826 | A_P329L_strat2 | 2.4E−07 | 8.2E−07 | 6.1 | 0.1 | 0.4 | 0.1 | 16.2 | 1.2 | 75.31 |
| | 26827 | A_P329I_strat2 | 5.7E−08 | 2.5E−07 | 25.4 | 0.3 | 1.3 | 0.2 | 20.3 | 1.5 | 77.07 |
| | 26828 | A_P329F_strat2 | ND | ND | — | — | — | — | — | — | 78.67 |
| | 26829 | A_P329W_strat2 | ND | ND | — | — | — | — | — | — | 52.96 |
| | 26830 | A_P329Y_strat2 | ND | ND | — | — | — | — | — | — | 116.54 |
| | 26831 | A_P329T_strat2 | ND | ND | — | — | — | — | — | — | −109.94 |
| | 26832 | A_P329S_strat2 | ND | ND | — | — | — | — | — | — | 299.89 |
| | 26833 | A_P329Q_strat2 | ND | ND | — | — | — | — | — | — | −85.28 |
| | 26834 | A_P329D_strat2 | ND | ND | — | — | — | — | — | — | −68.05 |
| | 26835 | A_P329E_strat2 | ND | ND | — | — | — | — | — | — | −111.86 |
| | 26836 | A_P329R_strat2 | ND | ND | — | — | — | — | — | — | −55.42 |
| | 26837 | A_P329K_strat2 | ND | ND | — | — | — | — | — | — | −34.09 |
| | 26838 | A_P329H_strat2 | ND | ND | — | — | — | — | — | — | 205.80 |
| | 26839 | A_K330G_strat2 | 6.1E−08 | 1.8E−07 | 23.8 | 0.3 | 1.7 | 0.2 | 14.2 | 1.1 | 39.88 |
| | 26840 | A_K330A_strat2 | 2.5E−08 | 8.1E−08 | 58.0 | 0.6 | 3.8 | 0.5 | 15.3 | 1.1 | 48.01 |
| | 26841 | A_K330V_strat2 | 3.8E−08 | 1.3E−07 | 37.8 | 0.4 | 2.4 | 0.3 | 15.9 | 1.2 | 67.67 |
| | 26842 | A_K330L_strat2 | 5.5E−08 | 1.6E−07 | 26.3 | 0.3 | 1.9 | 0.3 | 13.7 | 1.0 | 59.44 |
| | 26843 | A_K330I_strat2 | 4.7E−08 | 1.5E−07 | 30.7 | 0.3 | 2.1 | 0.3 | 15.0 | 1.1 | 66.56 |
| | 26844 | A_K330F_strat2 | 5.2E−08 | 1.5E−07 | 27.7 | 0.3 | 2.1 | 0.3 | 13.4 | 1.0 | 60.43 |
| | 26845 | A_K330W_strat2 | 2.5E−07 | 5.8E−07 | 5.9 | 0.1 | 0.5 | 0.1 | 11.1 | 0.8 | 56.18 |
| | 26846 | A_K330Y_strat2 | 6.1E−08 | 1.9E−07 | 23.7 | 0.3 | 1.7 | 0.2 | 14.3 | 1.1 | 32.74 |
| | 26847 | A_K330T_strat2 | 2.7E−08 | 1.3E−07 | 53.3 | 0.6 | 2.4 | 0.3 | 22.6 | 1.7 | 61.79 |
| | 26848 | A_K330S_strat2 | 2.8E−08 | 8.4E−08 | 51.6 | 0.6 | 3.7 | 0.5 | 14.0 | 1.0 | 71.32 |
| | 26849 | A_K330Q_strat2 | 2.7E−08 | 9.9E−08 | 53.5 | 0.6 | 3.1 | 0.4 | 17.2 | 1.3 | 62.44 |
| | 26850 | A_K330N_strat2 | 5.4E−08 | 1.4E−07 | 26.7 | 0.3 | 2.2 | 0.3 | 12.2 | 0.9 | 59.10 |
| | 26851 | A_K330D_strat2 | 3.7E−08 | 1.1E−07 | 39.2 | 0.4 | 2.9 | 0.4 | 13.6 | 1.0 | 56.10 |
| | 26852 | A_K330E_strat2 | 5.9E−08 | 1.7E−07 | 24.5 | 0.3 | 1.8 | 0.3 | 13.6 | 1.0 | 51.63 |
| | 26853 | A_K330R_strat2 | 2.3E−08 | 7.2E−08 | 62.6 | 0.7 | 4.3 | 0.6 | 14.6 | 1.1 | 61.27 |
| | 26854 | A_K330H_strat2 | 2.9E−08 | 7.5E−08 | 50.0 | 0.5 | 4.1 | 0.6 | 12.2 | 0.9 | 52.38 |
| | 26855 | A_K330P_strat2 | 5.0E−07 | 3.6E−07 | 2.9 | 0.0 | 0.9 | 0.1 | 3.4 | 0.3 | 21.52 |
| | 26856 | A_S331G_strat2 | 7.5E−08 | 2.4E−07 | 19.4 | 0.2 | 1.3 | 0.2 | 14.8 | 1.1 | 77.73 |
| | 26857 | A_S331A_strat2 | 1.6E−08 | 4.8E−08 | 88.7 | 1.0 | 6.4 | 0.9 | 13.8 | 1.0 | 55.33 |
| | 26858 | A_S331V_strat2 | 5.0E−08 | 1.5E−07 | 29.1 | 0.3 | 2.1 | 0.3 | 13.9 | 1.0 | 58.97 |
| | 26859 | A_S331L_strat2 | 4.6E−08 | 1.5E−07 | 31.3 | 0.3 | 2.1 | 0.3 | 14.7 | 1.1 | 62.17 |
| | 26860 | A_S331I_strat2 | 6.3E−08 | 1.8E−07 | 23.1 | 0.2 | 1.7 | 0.2 | 13.5 | 1.0 | 65.91 |
| | 26861 | A_S331F_strat2 | 4.4E−08 | 1.3E−07 | 32.5 | 0.4 | 2.3 | 0.3 | 14.0 | 1.0 | 57.97 |
| | 26862 | A_S331W_strat2 | 3.9E−08 | 1.1E−07 | 37.4 | 0.4 | 2.7 | 0.4 | 13.8 | 1.0 | 59.35 |
| | 26863 | A_S331Y_strat2 | 3.9E−08 | 1.2E−07 | 36.8 | 0.4 | 2.7 | 0.4 | 13.8 | 1.0 | 56.82 |
| | 26864 | A_S331T_strat2 | 3.7E−08 | 1.2E−07 | 38.8 | 0.4 | 2.6 | 0.4 | 14.9 | 1.1 | 59.14 |
| | 26865 | A_S331Q_strat2 | 2.4E−08 | 6.9E−08 | 60.5 | 0.7 | 4.5 | 0.6 | 13.6 | 1.0 | 53.24 |
| | 26866 | A_S331N_strat2 | 3.6E−08 | 1.0E−07 | 40.6 | 0.4 | 3.1 | 0.4 | 13.1 | 1.0 | 38.50 |
| | 26867 | A_S331D_strat2 | 2.4E−08 | 6.5E−08 | 61.5 | 0.7 | 4.7 | 0.7 | 13.0 | 1.0 | 57.04 |
| | 26868 | A_S331E_strat2 | 2.3E−08 | 5.9E−08 | 63.7 | 0.7 | 5.2 | 0.7 | 12.2 | 0.9 | 54.76 |
| | 26869 | A_S331R_strat2 | 4.4E−08 | 1.2E−07 | 32.9 | 0.4 | 2.7 | 0.4 | 12.4 | 0.9 | 47.39 |
| | 26870 | A_S331K_strat2 | 3.8E−08 | 1.2E−07 | 37.8 | 0.4 | 2.6 | 0.4 | 14.6 | 1.1 | 62.71 |
| | 26871 | A_S331H_strat2 | 2.6E−08 | 8.5E−08 | 54.7 | 0.6 | 3.6 | 0.5 | 15.0 | 1.1 | 41.07 |
| | 26872 | A_S331P_strat2 | 1.3E−08 | 3.5E−08 | 113.8 | 1.2 | 8.9 | 1.3 | 12.8 | 1.0 | 61.26 |
| | 26873 | A_1332A_strat2 | 1.6E−08 | 4.6E−08 | 92.0 | 1.0 | 6.7 | 1.0 | 13.8 | 1.0 | 48.91 |
| | 26874 | A_1332V_strat2 | 2.2E−08 | 6.8E−08 | 65.4 | 0.7 | 4.5 | 0.7 | 14.4 | 1.1 | 52.02 |
| | 26875 | A_1332L_strat2 | 2.8E−08 | 7.8E−08 | 52.6 | 0.6 | 3.9 | 0.6 | 13.3 | 1.0 | 46.62 |
| | 26876 | A_1332M_strat2 | 1.9E−08 | 4.5E−08 | 76.9 | 0.8 | 6.8 | 1.0 | 11.3 | 0.8 | 40.71 |
| | 26877 | A_1332F_strat2 | 3.6E−08 | 9.0E−08 | 40.3 | 0.4 | 3.4 | 0.5 | 11.8 | 0.9 | 43.17 |
| | 26878 | A_1332T_strat2 | 1.8E−08 | 4.6E−08 | 82.6 | 0.9 | 6.7 | 1.0 | 12.3 | 0.9 | −0.43 |
| Strat2 | 26879 | B_L234G_strat2 | 2.3E−08 | 4.9E−08 | 62.3 | 0.7 | 6.3 | 0.9 | 9.8 | 0.7 | 31.67 |
| Chain B | 26880 | B_L234A_strat2 | 2.4E−08 | 6.8E−08 | 60.7 | 0.7 | 4.5 | 0.7 | 13.3 | 1.0 | 56.46 |
| | 26881 | B_L234V_strat2 | 2.6E−08 | 7.4E−08 | 56.2 | 0.6 | 4.2 | 0.6 | 13.5 | 1.0 | 58.76 |
| | 26882 | B_L234I_strat2 | 2.0E−08 | 6.4E−08 | 72.6 | 0.8 | 4.8 | 0.7 | 15.1 | 1.1 | 60.58 |
| | 26883 | B_L234F_strat2 | 2.1E−08 | 5.7E−08 | 67.8 | 0.7 | 5.4 | 0.8 | 12.6 | 0.9 | 48.92 |
| | 26884 | B_L234W_strat2 | 2.5E−08 | 7.2E−08 | 57.1 | 0.6 | 4.3 | 0.6 | 13.3 | 1.0 | 65.05 |
| | 26885 | B_L234Y_strat2 | 2.6E−08 | 6.4E−08 | 56.5 | 0.6 | 4.8 | 0.7 | 11.7 | 0.9 | 44.00 |
| | 26886 | B_L234T_strat2 | 2.9E−08 | 7.5E−08 | 50.2 | 0.5 | 4.1 | 0.6 | 12.3 | 0.9 | 47.63 |
| | 26887 | B_L234S_strat2 | 2.5E−08 | 6.0E−08 | 58.5 | 0.6 | 5.2 | 0.7 | 11.3 | 0.8 | 32.64 |
| | 26888 | B_L234Q_strat2 | 2.8E−08 | 7.8E−08 | 51.4 | 0.6 | 4.0 | 0.6 | 12.9 | 1.0 | 39.23 |
| | 26889 | B_L234N_strat2 | 2.4E−08 | 5.5E−08 | 60.5 | 0.7 | 5.6 | 0.8 | 10.7 | 0.8 | 44.20 |
| | 26890 | B_L234D_strat2 | 2.7E−08 | 6.8E−08 | 54.1 | 0.6 | 4.5 | 0.7 | 11.9 | 0.9 | 43.75 |
| | 26891 | B_L234E_strat2 | 2.9E−08 | 8.7E−08 | 49.5 | 0.5 | 3.5 | 0.5 | 14.0 | 1.0 | 41.16 |
| | 26892 | B_L234R_strat2 | 3.2E−08 | 7.5E−08 | 45.7 | 0.5 | 4.1 | 0.6 | 11.1 | 0.8 | 36.92 |
| | 26893 | B_L234K_strat2 | 3.9E−08 | 1.1E−07 | 37.5 | 0.4 | 2.9 | 0.4 | 13.0 | 1.0 | |
| | 26894 | B_L234H_strat2 | 3.9E−08 | 8.9E−08 | 37.1 | 0.4 | 3.5 | 0.5 | 10.7 | 0.8 | 38.05 |
| | 26895 | B_L234P_strat2 | 1.8E−08 | 5.7E−08 | 79.8 | 0.9 | 5.4 | 0.8 | 14.8 | 1.1 | 52.48 |
| | 26896 | B_L235G_strat2 | 2.6E−08 | 6.0E−08 | 55.4 | 0.6 | 5.1 | 0.7 | 10.8 | 0.8 | 48.65 |
| | 26897 | B_L235A_strat2 | 2.5E−08 | 6.3E−08 | 58.3 | 0.6 | 4.9 | 0.7 | 12.0 | 0.9 | −0.56 |
| | 26898 | B_L235V_strat2 | 4.7E−08 | 1.0E−07 | 30.5 | 0.3 | 3.0 | 0.4 | 10.2 | 0.8 | 43.46 |
| | 26899 | B_L235I_strat2 | 3.7E−08 | 8.7E−08 | 39.6 | 0.4 | 3.5 | 0.5 | 11.2 | 0.8 | 40.29 |
| | 26900 | B_L235F_strat2 | 1.2E−08 | 3.2E−08 | 121.4 | 1.3 | 9.6 | 1.4 | 12.7 | 1.0 | 50.93 |
| | 26901 | B_L235W_strat2 | 1.9E−08 | 4.1E−08 | 77.8 | 0.8 | 7.5 | 1.1 | 10.3 | 0.8 | |
| | 26902 | B_L235Y_strat2 | 1.2E−08 | 3.1E−08 | 117.5 | 1.3 | 10.0 | 1.4 | 11.7 | 0.9 | 45.25 |
| | 26903 | B_L235T_strat2 | 4.3E−08 | 1.1E−07 | 33.3 | 0.4 | 2.7 | 0.4 | 12.2 | 0.9 | 24.63 |
| | 26904 | B_L235S_strat2 | 3.7E−08 | 1.0E−07 | 39.1 | 0.4 | 3.0 | 0.4 | 13.0 | 1.0 | 37.33 |
| | 26905 | B_L235Q_strat2 | 3.2E−08 | 9.1E−08 | 44.6 | 0.5 | 3.4 | 0.5 | 13.2 | 1.0 | 55.00 |
| | 26906 | B_L235N_strat2 | 1.4E−08 | 3.9E−08 | 100.4 | 1.1 | 7.9 | 1.1 | 12.7 | 0.9 | 49.93 |
| | 26907 | B_L235D_strat2 | 1.8E−08 | 6.7E−08 | 81.6 | 0.9 | 4.6 | 0.7 | 17.7 | 1.3 | 63.78 |
| | 26908 | B_L235E_strat2 | 3.8E−08 | 1.2E−07 | 38.1 | 0.4 | 2.5 | 0.4 | 15.3 | 1.1 | 47.56 |
| | 26909 | B_L235R_strat2 | 6.6E−08 | 1.5E−07 | 21.8 | 0.2 | 2.0 | 0.3 | 10.9 | 0.8 | −1.02 |
| | 26910 | B_L235K_strat2 | 7.2E−08 | 2.0E−07 | 20.2 | 0.2 | 1.6 | 0.2 | 12.8 | 1.0 | 39.85 |
| | 26911 | B_L235H_strat2 | 2.7E−08 | 6.4E−08 | 53.3 | 0.6 | 4.8 | 0.7 | 11.1 | 0.8 | 46.51 |
| | 26912 | B_L235P_strat2 | 3.5E−08 | 7.5E−08 | 40.8 | 0.4 | 4.1 | 0.6 | 9.9 | 0.7 | 33.90 |
| | 27292 | B_D236G_strat2 | 2.7E−08 | 2.3E−08 | 52.9 | 0.6 | 13.6 | 2.0 | 3.9 | 0.3 | 4.19 |
| | 26913 | B_D236A_strat2 | 3.0E−08 | 2.4E−08 | 47.9 | 0.5 | 12.8 | 1.8 | 3.7 | 0.3 | −0.45 |
| | 26914 | B_D236V_strat2 | 4.6E−08 | 7.7E−08 | 31.2 | 0.3 | 4.0 | 0.6 | 7.8 | 0.6 | −0.73 |
| | 26915 | B_D236L_strat2 | 5.5E−08 | 1.2E−07 | 26.5 | 0.3 | 2.5 | 0.4 | 10.7 | 0.8 | 33.53 |
| | 26916 | B_D236I_strat2 | 5.2E−08 | 9.0E−08 | 27.6 | 0.3 | 3.4 | 0.5 | 8.0 | 0.6 | 17.02 |
| | 26917 | B_D236F_strat2 | 8.8E−08 | 1.1E−07 | 16.4 | 0.2 | 2.7 | 0.4 | 6.1 | 0.5 | 12.18 |
| | 26918 | B_D236W_strat2 | 5.2E−08 | 5.1E−08 | 28.0 | 0.3 | 6.1 | 0.9 | 4.6 | 0.3 | 15.01 |
| | 26919 | B_D236Y_strat2 | 5.8E−08 | 7.1E−08 | 24.9 | 0.3 | 4.3 | 0.6 | 5.8 | 0.4 | 8.09 |
| | 26920 | B_D236T_strat2 | 5.0E−08 | 8.1E−08 | 29.2 | 0.3 | 3.8 | 0.5 | 7.7 | 0.6 | 10.21 |
| | 26921 | B_D236S_strat2 | 3.5E−08 | 3.5E−08 | 41.3 | 0.4 | 8.7 | 1.3 | 4.7 | 0.4 | 4.24 |
| | 26922 | B_D236Q_strat2 | 4.9E−08 | 8.1E−08 | 29.7 | 0.3 | 3.8 | 0.5 | 7.8 | 0.6 | 9.64 |
| | 26923 | B_D236N_strat2 | 6.7E−08 | 1.7E−07 | 21.7 | 0.2 | 1.8 | 0.3 | 12.2 | 0.9 | 37.11 |
| | 26924 | B_D236E_strat2 | 2.3E−08 | 3.6E−08 | 63.7 | 0.7 | 8.6 | 1.2 | 7.4 | 0.6 | 5.75 |
| | 26925 | B_D236R_strat2 | 2.6E−07 | 4.5E−07 | 5.5 | 0.1 | 0.7 | 0.1 | 7.9 | 0.6 | 28.44 |
| | 26926 | B_D236K_strat2 | 3.5E−07 | 6.6E−07 | 4.2 | 0.0 | 0.5 | 0.1 | 8.9 | 0.7 | −2.00 |
| | 26927 | B_D236H_strat2 | 1.9E−07 | 1.8E−07 | 7.5 | 0.1 | 1.7 | 0.2 | 4.4 | 0.3 | 4.96 |
| | 26928 | B_D236P_strat2 | 1.3E−07 | 1.5E−07 | 11.2 | 0.1 | 2.0 | 0.3 | 5.6 | 0.4 | 2.58 |
| | 26929 | B_G237A_strat2 | 6.5E−09 | 2.6E−08 | 223.7 | 2.4 | 12.0 | 1.7 | 18.7 | 1.4 | 69.70 |
| | 26930 | B_G237V_strat2 | 1.9E−08 | 6.0E−08 | 75.3 | 0.8 | 5.1 | 0.7 | 14.7 | 1.1 | 66.79 |
| | 26931 | B_G237L_strat2 | 4.9E−09 | 2.2E−08 | 296.1 | 3.2 | 14.2 | 2.0 | 20.9 | 1.6 | 72.52 |
| | 26932 | B_G237I_strat2 | 1.7E−08 | 6.4E−08 | 87.1 | 0.9 | 4.8 | 0.7 | 18.0 | 1.3 | 72.36 |
| | 26933 | B_G237F_strat2 | 9.7E−09 | 3.5E−08 | 148.4 | 1.6 | 8.7 | 1.3 | 17.0 | 1.3 | |
| | 26934 | B_G237W_strat2 | 6.5E−09 | 2.5E−08 | 224.1 | 2.4 | 12.4 | 1.8 | 18.1 | 1.4 | 74.68 |
| | 26935 | B_G237Y_strat2 | 6.5E−09 | 2.4E−08 | 220.7 | 2.4 | 12.7 | 1.8 | 17.4 | 1.3 | |
| | 26936 | B_G237T_strat2 | 1.6E−08 | 5.6E−08 | 91.5 | 1.0 | 5.5 | 0.8 | 16.6 | 1.2 | 67.25 |
| | 26937 | B_G237S_strat2 | 1.0E−08 | 2.6E−08 | 141.4 | 1.5 | 11.8 | 1.7 | 12.0 | 0.9 | |
| | 26938 | B_G237Q_strat2 | 4.0E−09 | 1.4E−08 | 358.6 | 3.9 | 22.4 | 3.2 | 16.0 | 1.2 | 55.74 |
| | 26939 | B_G237N_strat2 | 2.7E−09 | 1.1E−08 | 541.7 | 5.8 | 27.5 | 4.0 | 19.7 | 1.5 | |
| | 26940 | B_G237D_strat2 | 4.8E−09 | 2.4E−08 | 304.2 | 3.3 | 13.1 | 1.9 | 23.3 | 1.7 | 65.87 |
| | 26941 | B_G237E_strat2 | 1.0E−08 | 3.4E−08 | 143.1 | 1.5 | 9.0 | 1.3 | 15.9 | 1.2 | 63.89 |
| | 26942 | B_G237R_strat2 | 2.8E−08 | 8.8E−08 | 51.4 | 0.6 | 3.5 | 0.5 | 14.7 | 1.1 | 57.02 |
| | 26943 | B_G237K_strat2 | 1.3E−08 | 3.8E−08 | 115.6 | 1.2 | 8.1 | 1.2 | 14.4 | 1.1 | 57.00 |
| | 26944 | B_G237H_strat2 | 2.0E−08 | 5.7E−08 | 73.4 | 0.8 | 5.4 | 0.8 | 13.5 | 1.0 | 42.41 |
| | 26945 | B_G237P_strat2 | 1.0E−07 | 3.7E−07 | 14.0 | 0.2 | 0.8 | 0.1 | 16.9 | 1.3 | 69.45 |
| | 26946 | B_D239G_strat2 | 3.1E−08 | 9.9E−08 | 46.2 | 0.5 | 3.1 | 0.4 | 14.8 | 1.1 | 53.87 |
| | 26947 | B_D239A_strat2 | 9.8E−08 | 2.3E−07 | 14.8 | 0.2 | 1.3 | 0.2 | 11.1 | 0.8 | 34.97 |
| | 26948 | B_D239V_strat2 | 5.1E−08 | 1.1E−07 | 28.4 | 0.3 | 2.8 | 0.4 | 10.3 | 0.8 | 26.56 |
| | 26949 | B_D239L_strat2 | 2.7E−08 | 6.1E−08 | 54.1 | 0.6 | 5.0 | 0.7 | 10.7 | 0.8 | 41.72 |
| | 26950 | B_D239I_strat2 | 4.8E−08 | 9.6E−08 | 30.4 | 0.3 | 3.2 | 0.5 | 9.4 | 0.7 | 29.97 |
| | 26951 | B_D239F_strat2 | 8.8E−08 | 9.8E−08 | 16.4 | 0.2 | 3.1 | 0.5 | 5.2 | 0.4 | 26.36 |
| | 26952 | B_D239W_strat2 | 9.2E−08 | 2.1E−07 | 15.8 | 0.2 | 1.5 | 0.2 | 10.5 | 0.8 | 38.74 |
| | 26953 | B_D239Y_strat2 | 1.4E−07 | 3.2E−07 | 10.5 | 0.1 | 1.0 | 0.1 | 10.9 | 0.8 | 48.23 |
| | 26954 | B_D239T_strat2 | 8.8E−08 | 2.3E−07 | 16.5 | 0.2 | 1.3 | 0.2 | 12.2 | 0.9 | 45.42 |
| | 26955 | B_D239S_strat2 | 1.2E−07 | 3.0E−07 | 12.2 | 0.1 | 1.0 | 0.1 | 11.9 | 0.9 | 49.85 |
| | 26956 | B_D239Q_strat2 | 1.3E−07 | 3.0E−07 | 11.2 | 0.1 | 1.0 | 0.1 | 11.0 | 0.8 | 34.32 |
| | 26957 | B_D239N_strat2 | 5.8E−08 | 1.3E−07 | 24.8 | 0.3 | 2.3 | 0.3 | 10.7 | 0.8 | 44.88 |
| | 26958 | B_D239E_strat2 | 4.2E−08 | 1.1E−07 | 34.2 | 0.4 | 2.9 | 0.4 | 11.6 | 0.9 | 46.05 |
| | 26959 | B_D239R_strat2 | 4.6E−07 | 8.9E−07 | 3.1 | 0.0 | 0.3 | 0.0 | 9.0 | 0.7 | 45.29 |
| | 26960 | B_D239K_strat2 | ND | ND | — | — | — | — | — | — | |
| | 26961 | B_D239H_strat2 | 3.1E−07 | 6.3E−07 | 4.7 | 0.1 | 0.5 | 0.1 | 9.6 | 0.7 | 55.35 |
| | 26962 | B_D239P_strat2 | 2.0E−07 | 5.3E−07 | 7.4 | 0.1 | 0.6 | 0.1 | 12.6 | 0.9 | 54.05 |
| | 26963 | B_V240A_strat2 | 6.8E−08 | 1.8E−07 | 21.2 | 0.2 | 1.7 | 0.2 | 12.4 | 0.9 | 44.84 |
| | 26964 | B_V240L_strat2 | 2.5E−08 | 7.4E−08 | 57.6 | 0.6 | 4.2 | 0.6 | 13.8 | 1.0 | 61.20 |
| | 26965 | B_V240I_strat2 | 2.5E−08 | 7.9E−08 | 58.0 | 0.6 | 3.9 | 0.6 | 14.9 | 1.1 | |
| | 26966 | B_V240M_strat2 | 3.0E−08 | 8.3E−08 | 48.8 | 0.5 | 3.7 | 0.5 | 13.1 | 1.0 | 54.86 |
| | 26967 | B_V240F_strat2 | 8.0E−08 | 1.7E−07 | 18.0 | 0.2 | 1.8 | 0.3 | 9.8 | 0.7 | |
| | 26968 | B_V240T_strat2 | 3.0E−08 | 5.9E−08 | 48.4 | 0.5 | 5.2 | 0.7 | 9.3 | 0.7 | 51.20 |
| | 26969 | B_V263A_strat2 | 4.5E−08 | 8.6E−08 | 32.4 | 0.3 | 3.6 | 0.5 | 9.1 | 0.7 | 46.67 |
| | 26970 | B_V263L_strat2 | 7.4E−08 | 1.5E−07 | 19.6 | 0.2 | 2.0 | 0.3 | 9.6 | 0.7 | 49.67 |
| | 26971 | B_V263I_strat2 | 4.2E−08 | 1.0E−07 | 34.3 | 0.4 | 3.1 | 0.4 | 11.1 | 0.8 | |
| | 26972 | B_V263M_strat2 | 6.2E−08 | 1.1E−07 | 23.2 | 0.3 | 2.9 | 0.4 | 8.0 | 0.6 | 51.30 |
| | 26973 | B_V263F_strat2 | ND | ND | — | — | — | — | — | — | −39.91 |
| | 26974 | B_V263T_strat2 | 3.6E−08 | 6.9E−08 | 40.0 | 0.4 | 4.5 | 0.6 | 9.0 | 0.7 | 65.85 |
| | 26975 | B_V264A_strat2 | 3.2E−08 | 7.7E−08 | 45.7 | 0.5 | 4.0 | 0.6 | 11.5 | 0.9 | 64.52 |
| | 26976 | B_V264L_strat2 | 1.4E−08 | 5.3E−08 | 104.4 | 1.1 | 5.8 | 0.8 | 18.0 | 1.3 | |
| | 26977 | B_V264I_strat2 | 3.6E−08 | 8.2E−08 | 40.7 | 0.4 | 3.8 | 0.5 | 10.8 | 0.8 | 55.59 |
| | 26978 | B_V264M_strat2 | 3.8E−08 | 8.5E−08 | 38.0 | 0.4 | 3.6 | 0.5 | 10.5 | 0.8 | 62.64 |
| | 26979 | B_V264F_strat2 | 7.4E−08 | 1.6E−07 | 19.6 | 0.2 | 2.0 | 0.3 | 10. | 0.7 | 51.65 |
| | 26980 | B_V264T_strat2 | 1.2E−08 | 2.3E−08 | 121.2 | 1.3 | 13.7 | 2.0 | 8.9 | 0.7 | 59.12 |
| | 26981 | B_L266V_strat2 | 3.0E−08 | 7.7E−08 | 48.2 | 0.5 | 4.0 | 0.6 | 12.1 | 0.9 | 38.28 |
| | 26982 | B_L266A_strat2 | ND | ND | — | — | — | — | — | — | 46.65 |
| | 26983 | B_L266I_strat2 | 2.5E−08 | 4.3E−08 | 58.7 | 0.6 | 7.1 | 1.0 | 8.3 | 0.6 | 54.23 |
| | 26984 | B_L266M_strat2 | 2.5E−08 | 5.5E−08 | 57.3 | 0.6 | 5.6 | 0.8 | 10.3 | 0.8 | 63.16 |
| | 26985 | B_L266F_strat2 | 1.5E−07 | 3.2E−07 | 9.9 | 0.1 | 1.0 | 0.1 | 10.1 | 0.8 | 57.89 |
| | 26986 | B_L266T_strat2 | 4.1E−07 | 7.2E−07 | 3.6 | 0.0 | 0.4 | 0.1 | 8.3 | 0.6 | 92.81 |
| | 26987 | B_A267G_strat2 | 6.0E−08 | 9.8E−08 | 24.1 | 0.3 | 3.1 | 0.5 | 7.7 | 0.6 | 32.75 |
| | 26988 | B_A267V_strat2 | 7.9E−08 | 1.6E−07 | 18.2 | 0.2 | 1.9 | 0.3 | 9.4 | 0.7 | 58.80 |
| | 26989 | B_A267L_strat2 | 2.7E−07 | 4.9E−07 | 5.3 | 0.1 | 0.6 | 0.1 | 8.3 | 0.6 | 52.75 |
| | 26990 | B_A267I_strat2 | 1.2E−07 | 3.0E−07 | 12.0 | 0.1 | 1.0 | 0.2 | 11.5 | 0.9 | 38.44 |
| | 26991 | B_A267F_strat2 | 5.3E−07 | 1.2E−06 | 2.7 | 0.0 | 0.3 | 0.0 | 10.7 | 0.8 | 69.78 |
| | 26992 | B_A267W_strat2 | 6.8E−07 | 1.7E−06 | 2.1 | 0.0 | 0.2 | 0.0 | 11.5 | 0.9 | 63.91 |
| | 26993 | B_A267Y_strat2 | 7.7E−07 | 1.3E−06 | 1.9 | 0.0 | 0.2 | 0.0 | 8.0 | 0.6 | 84.07 |
| | 26994 | B_A267T_strat2 | 5.6E−08 | 2.0E−07 | 25.9 | 0.3 | 1.5 | 0.2 | 17.0 | 1.3 | |
| | 26995 | B_A267S_strat2 | 3.3E−08 | 1.0E−07 | 44.5 | 0.5 | 3.1 | 0.4 | 14.6 | 1.1 | 46.99 |
| | 26996 | B_A267Q_strat2 | 1.4E−08 | 3.1E−08 | 105.1 | 1.1 | 10.0 | 1.4 | 10.5 | 0.8 | 62.25 |
| | 26997 | B_A267N_strat2 | 1.4E−07 | 3.8E−07 | 10.2 | 0.1 | 0.8 | 0.1 | 12.4 | 0.9 | 69.46 |
| | 26999 | B_A267R_strat2 | ND | ND | — | — | — | — | — | — | −4.80 |
| | 27002 | B_A267K_strat2 | ND | ND | — | — | — | — | — | — | −19.89 |
| | 27003 | B_A267H_strat2 | 1.8E−07 | 5.4E−07 | 8.0 | 0.1 | 0.6 | 0.1 | 14.1 | 1.1 | 81.66 |
| | 27006 | B_A267P_strat2 | ND | ND | — | — | — | — | — | — | 104.59 |
| | 27008 | B_D268G_strat2 | 5.8E−08 | 9.4E−08 | 24.8 | 0.3 | 3.3 | 0.5 | 7.6 | 0.6 | 36.98 |
| | 27010 | B_D268A_strat2 | 2.4E−08 | 4.3E−08 | 61.5 | 0.7 | 7.2 | 1.0 | 8.5 | 0.6 | 41.44 |
| | 27012 | B_D268V_strat2 | 2.4E−08 | 4.7E−08 | 60.9 | 0.7 | 6.6 | 1.0 | 9.2 | 0.7 | 45.77 |
| | 27014 | B_D268L_strat2 | 5.4E−08 | 1.1E−07 | 26.6 | 0.3 | 2.9 | 0.4 | 9.1 | 0.7 | 40.91 |
| | 27016 | B_D268I_strat2 | ND | ND | — | — | — | — | — | — | |
| | 27019 | B_D268F_strat2 | 2.6E−08 | 4.6E−08 | 56.5 | 0.6 | 6.7 | 1.0 | 8.4 | 0.6 | 46.94 |
| | 27021 | B_D268W_strat2 | 2.6E−08 | 4.6E−08 | 55.1 | 0.6 | 6.7 | 1.0 | 8.3 | 0.6 | 47.80 |
| | 27023 | B_D268Y_strat2 | 2.4E−08 | 4.5E−08 | 60.5 | 0.7 | 6.9 | 1.0 | 8.8 | 0.7 | 53.41 |
| | 27026 | B_D268T_strat2 | 4.7E−08 | 8.3E−08 | 30.7 | 0.3 | 3.7 | 0.5 | 8.2 | 0.6 | 40.35 |
| | 27027 | B_D268S_strat2 | 2.8E−08 | 4.8E−08 | 52.0 | 0.6 | 6.4 | 0.9 | 8.1 | 0.6 | 42.74 |
| | 27029 | B_D268Q_strat2 | 2.0E−08 | 3.7E−08 | 73.9 | 0.8 | 8.3 | 1.2 | 8.9 | 0.7 | 47.79 |
| | 27031 | B_D268N_strat2 | 3.0E−08 | 5.5E−08 | 48.3 | 0.5 | 5.7 | 0.8 | 8.5 | 0.6 | 41.70 |
| | 27033 | B_D268E_strat2 | 1.8E−08 | 3.7E−08 | 81.3 | 0.9 | 8.3 | 1.2 | 9.8 | 0.7 | 62.51 |
| | 27035 | B_D268R_strat2 | 4.2E−08 | 5.4E−08 | 34.2 | 0.4 | 5.7 | 0.8 | 6.0 | 0.4 | 30.84 |
| | 27037 | B_D268K_strat2 | 3.8E−08 | 5.2E−08 | 38.3 | 0.4 | 5.9 | 0.8 | 6.5 | 0.5 | 31.08 |
| | 27039 | B_D268H_strat2 | 3.3E−08 | 8.0E−08 | 43.8 | 0.5 | 3.9 | 0.6 | 11.3 | 0.8 | 33.30 |
| | 27041 | B_D268P_strat2 | 8.3E−08 | 1.4E−07 | 17.4 | 0.2 | 2.3 | 0.3 | 7.7 | 0.6 | 42.89 |
| | 27043 | B_E269G_strat2 | 6.5E−08 | 1.4E−07 | 22.3 | 0.2 | 2.2 | 0.3 | 10.1 | 0.8 | 56.52 |
| | 27046 | B_E269A_strat2 | 5.7E−08 | 1.3E−07 | 25.5 | 0.3 | 2.4 | 0.3 | 10.6 | 0.8 | 56.60 |
| | 27047 | B_E269V_strat2 | 2.5E−08 | 6.7E−08 | 57.5 | 0.6 | 4.6 | 0.7 | 12.6 | 0.9 | 60.78 |
| | 27050 | B_E269L_strat2 | 4.9E−08 | 1.1E−07 | 29.3 | 0.3 | 2.8 | 0.4 | 10.4 | 0.8 | 60.36 |
| | 27052 | B_E269I_strat2 | 3.2E−08 | 7.7E−08 | 44.6 | 0.5 | 4.0 | 0.6 | 11.1 | 0.8 | 59.26 |
| | 27054 | B_E269F_strat2 | 1.3E−07 | 2.4E−07 | 11.2 | 0.1 | 1.3 | 0.2 | 8.8 | 0.7 | 60.08 |
| | 27055 | B_E269W_strat2 | 2.5E−07 | 4.9E−07 | 5.9 | 0.1 | 0.6 | 0.1 | 9.3 | 0.7 | 42.73 |
| | 27058 | B_E269Y_strat2 | 6.1E−08 | 1.6E−07 | 23.5 | 0.3 | 2.0 | 0.3 | 11.8 | 0.9 | 67.42 |
| | 27060 | B_E269T_strat2 | 2.5E−08 | 5.6E−08 | 58.0 | 0.6 | 5.5 | 0.8 | 10.5 | 0.8 | 57.23 |
| | 27062 | B_E269S_strat2 | 3.2E−08 | 7.3E−08 | 44.6 | 0.5 | 4.2 | 0.6 | 10.5 | 0.8 | 55.73 |
| | 27064 | B_E269Q_strat2 | 5.3E−08 | 1.2E−07 | 27.1 | 0.3 | 2.5 | 0.4 | 10.9 | 0.8 | 52.55 |
| | 27065 | B_E269N_strat2 | 3.7E−08 | 8.8E−08 | 38.7 | 0.4 | 3.5 | 0.5 | 11.0 | 0.8 | 61.68 |
| | 27068 | B_E269D_strat2 | 2.0E−08 | 4.2E−08 | 73.1 | 0.8 | 7.4 | 1.1 | 9.9 | 0.7 | 62.87 |
| | 27070 | B_E269R_strat2 | 1.5E−07 | 2.7E−07 | 9.7 | 0.1 | 1.1 | 0.2 | 8.6 | 0.6 | 47.94 |
| | 27071 | B_E269K_strat2 | 1.2E−07 | 2.5E−07 | 11.8 | 0.1 | 1.2 | 0.2 | 9.5 | 0.7 | 47.40 |
| | 27073 | B_E269H_strat2 | 8.7E−08 | 2.6E−07 | 16.7 | 0.2 | 1.2 | 0.2 | 14.0 | 1.0 | |
| | 27075 | B_E269P_strat2 | ND | ND | — | — | — | — | — | — | |
| | 27077 | B_D270G_strat2 | 1.0E−06 | 1.4E−06 | 1.4 | 0.0 | 0.2 | 0.0 | 6.7 | 0.5 | 75.49 |
| | 27079 | B_D270A_strat2 | 1.5E−06 | 1.8E−06 | 1.0 | 0.0 | 0.2 | 0.0 | 5.7 | 0.4 | 68.99 |
| | 27082 | B_D270V_strat2 | 1.4E−06 | 1.9E−06 | 1.0 | 0.0 | 0.2 | 0.0 | 6.2 | 0.5 | 83.58 |
| | 27083 | B_D270L_strat2 | ND | ND | — | — | — | — | — | — | 79.52 |
| | 27085 | B_D270I_strat2 | 7.8E−07 | 1.8E−06 | 1.8 | 0.0 | 0.2 | 0.0 | 10.8 | 0.8 | 149.41 |
| | 27088 | B_D270F_strat2 | 1.0E−06 | 2.1E−06 | 1.4 | 0.0 | 0.1 | 0.0 | 9.6 | 0.7 | 85.53 |
| | 27089 | B_D270W_strat2 | 1.1E−06 | 2.3E−06 | 1.3 | 0.0 | 0.1 | 0.0 | 9.6 | 0.7 | 480.67 |
| | 27092 | B_D270Y_strat2 | 6.1E−07 | 2.1E−06 | 2.4 | 0.0 | 0.1 | 0.0 | 16.3 | 1.2 | 112.92 |
| | 27094 | B_D270T_strat2 | 8.1E−07 | 1.1E−06 | 1.8 | 0.0 | 0.3 | 0.0 | 6.5 | 0.5 | 69.06 |
| | 27096 | B_D270S_strat2 | ND | ND | — | — | — | — | — | — | 84.17 |
| | 27098 | B_D270Q_strat2 | ND | ND | — | — | — | — | — | — | 184.16 |
| | 27100 | B_D270N_strat2 | ND | ND | — | — | — | — | — | — | |
| | 27103 | B_D270E_strat2 | 1.9E−07 | 3.2E−07 | 7.8 | 0.1 | 1.0 | 0.1 | 8.0 | 0.6 | 50.81 |
| | 27105 | B_D270R_strat2 | ND | ND | — | — | — | — | — | — | −80.47 |
| | 27108 | B_D270K_strat2 | ND | ND | — | — | — | — | — | — | −128.70 |
| | 27109 | B_D270H_strat2 | ND | ND | — | — | — | — | — | — | 181.14 |
| | 27111 | B_D270P_strat2 | 8.1E−07 | 1.9E−06 | 1.8 | 0.0 | 0.2 | 0.0 | 10.9 | 0.8 | 68.01 |
| | 27112 | B_P271G_strat2 | 1.4E−08 | 2.3E−08 | 104.0 | 1.1 | 13.7 | 2.0 | 7.6 | 0.6 | 31.94 |
| | 27115 | B_P271A_strat2 | 7.6E−08 | 1.5E−07 | 19.0 | 0.2 | 2.0 | 0.3 | 9.3 | 0.7 | 37.92 |
| | 27117 | B_P271V_strat2 | 8.1E−08 | 2.4E−07 | 17.9 | 0.2 | 1.3 | 0.2 | 13.7 | 1.0 | |
| | 27119 | B_P271L_strat2 | 3.5E−08 | 9.5E−08 | 41.4 | 0.4 | 3.2 | 0.5 | 12.7 | 1.0 | |
| | 27121 | B_P271I_strat2 | 1.4E−07 | 3.1E−07 | 10.6 | 0.1 | 1.0 | 0.1 | 10.8 | 0.8 | 52.99 |
| | 27123 | B_P271F_strat2 | 7.4E−08 | 1.4E−07 | 19.6 | 0.2 | 2.2 | 0.3 | 9.0 | 0.7 | 38.66 |
| | 27125 | B_P271W_strat2 | 8.8E−08 | 1.7E−07 | 16.5 | 0.2 | 1.8 | 0.3 | 8.9 | 0.7 | 40.09 |
| | 27128 | B_P271Y_strat2 | 7.1E−08 | 1.4E−07 | 20.5 | 0.2 | 2.2 | 0.3 | 9.4 | 0.7 | 44.53 |
| | 27131 | B_P271T_strat2 | 1.4E−07 | 2.9E−07 | 10.3 | 0.1 | 1.1 | 0.2 | 9.7 | 0.7 | 57.49 |
| | 27133 | B_P271S_strat2 | 3.1E−07 | 5.6E−07 | 4.6 | 0.1 | 0.5 | 0.1 | 8.5 | 0.6 | 52.19 |
| | 27135 | B_P271Q_strat2 | 6.2E−08 | 1.0E−07 | 23.2 | 0.2 | 3.0 | 0.4 | 7.8 | 0.6 | 40.18 |
| | 27137 | B_P271N_strat2 | 5.2E−08 | 8.3E−08 | 27.9 | 0.3 | 3.7 | 0.5 | 7.6 | 0.6 | 44.70 |
| | 27139 | B_P271D_strat2 | 5.8E−08 | 1.3E−07 | 24.8 | 0.3 | 2.5 | 0.4 | 10.0 | 0.8 | 47.98 |
| | 27141 | B_P271E_strat2 | 3.8E−08 | 7.4E−08 | 38.5 | 0.4 | 4.2 | 0.6 | 9.2 | 0.7 | 46.54 |
| | 27142 | B_P271R_strat2 | 3.4E−08 | 8.6E−08 | 42.5 | 0.5 | 3.6 | 0.5 | 11.9 | 0.9 | |
| | 27145 | B_P271K_strat2 | 3.6E−08 | 6.4E−08 | 39.8 | 0.4 | 4.8 | 0.7 | 8.3 | 0.6 | 39.51 |
| | 27147 | B_P271H_strat2 | 3.4E−08 | 9.0E−08 | 42.7 | 0.5 | 3.4 | 0.5 | 12.5 | 0.9 | |
| | 27149 | B_E272G_strat2 | 3.7E−08 | 8.7E−08 | 39.2 | 0.4 | 3.6 | 0.5 | 11.0 | 0.8 | |
| | 27151 | B_E272A_strat2 | 1.8E−08 | 5.0E−08 | 80.5 | 0.9 | 6.1 | 0.9 | 13.1 | 1.0 | |
| | 27152 | B_E272V_strat2 | 3.0E−08 | 6.3E−08 | 47.6 | 0.5 | 4.9 | 0.7 | 9.8 | 0.7 | 61.80 |
| | 27155 | B_E272L_strat2 | 2.2E−08 | 4.8E−08 | 66.0 | 0.7 | 6.5 | 0.9 | 10.2 | 0.8 | 58.89 |
| | 27157 | B_E272I_strat2 | 2.6E−08 | 5.4E−08 | 55.7 | 0.6 | 5.7 | 0.8 | 9.7 | 0.7 | 59.91 |
| | 27159 | B_E272F_strat2 | 3.9E−08 | 8.4E−08 | 37.2 | 0.4 | 3.7 | 0.5 | 10.2 | 0.8 | 53.55 |
| | 27162 | B_E272W_strat2 | 4.8E−08 | 1.1E−07 | 30.0 | 0.3 | 2.9 | 0.4 | 10.5 | 0.8 | 53.99 |
| | 27164 | B_E272Y_strat2 | 5.3E−08 | 1.0E−07 | 27.0 | 0.3 | 3.0 | 0.4 | 9.1 | 0.7 | 45.51 |
| | 27166 | B_E272T_strat2 | 3.1E−08 | 5.9E−08 | 46.9 | 0.5 | 5.2 | 0.7 | 9.0 | 0.7 | 53.17 |
| | 27168 | B_E272S_strat2 | 3.6E−08 | 7.4E−08 | 40.6 | 0.4 | 4.1 | 0.6 | 9.8 | 0.7 | 52.15 |
| | 27170 | B_E272Q_strat2 | 2.7E−08 | 7.5E−08 | 54.5 | 0.6 | 4.1 | 0.6 | 13.2 | 1.0 | |
| | 27172 | B_E272N_strat2 | 4.1E−08 | 8.5E−08 | 34.8 | 0.4 | 3.6 | 0.5 | 9.6 | 0.7 | 53.06 |
| | 27174 | B_E272D_strat2 | 2.4E−08 | 4.9E−08 | 59.4 | 0.6 | 6.3 | 0.9 | 9.5 | 0.7 | 51.16 |
| | 27176 | B_E272R_strat2 | 2.4E−08 | 5.6E−08 | 61.2 | 0.7 | 5.5 | 0.8 | 11.2 | 0.8 | 54.87 |
| | 27178 | B_E272K_strat2 | 2.3E−08 | 5.0E−08 | 63.5 | 0.7 | 6.2 | 0.9 | 10.3 | 0.8 | 53.93 |
| | 27181 | B_E272H_strat2 | 4.2E−08 | 8.0E−08 | 34.2 | 0.4 | 3.8 | 0.6 | 8.9 | 0.7 | 52.16 |
| | 27182 | B_E272P_strat2 | 1.4E−08 | 2.7E−08 | 105.6 | 1.1 | 11.4 | 1.6 | 9.3 | 0.7 | 57.66 |
| | 27184 | B_V273A_strat2 | 2.9E−08 | 5.9E−08 | 50.4 | 0.5 | 5.3 | 0.8 | 9.6 | 0.7 | 54.89 |
| | 27186 | B_V273L_strat2 | 2.0E−08 | 6.1E−08 | 72.2 | 0.8 | 5.1 | 0.7 | 14.3 | 1.1 | |
| | 27189 | B_V273I_strat2 | 1.4E−08 | 3.2E−08 | 100.2 | 1.1 | 9.5 | 1.4 | 10.5 | 0.8 | 61.31 |
| | 27191 | B_V273M_strat2 | 1.4E−08 | 3.3E−08 | 104.8 | 1.1 | 9.4 | 1.4 | 11.1 | 0.8 | 66.13 |
| | 27193 | B_V273F_strat2 | 7.4E−08 | 1.9E−07 | 19.6 | 0.2 | 1.6 | 0.2 | 12.2 | 0.9 | |
| | 27195 | B_V273T_strat2 | 8.7E−09 | 1.9E−08 | 166.4 | 1.8 | 16.6 | 2.4 | 10.0 | 0.8 | 57.52 |

¹Mutation notation is in the format A_F234G_strat2, where “A” indicates the Fc chain, “F234G” indicates the mutation made with “F” representing the parental residue being replaced, 234 representing the position and G representing the replacement residue, and “strat2” specifies the parental CH2 mutations (A_L234F_G236N_H268Q_A327G_A330K_P331S/B_G236D_S239D_V266L_S267A_H268D)
²Selectivity is defined as IIb-Fold/IIaR-Fold
³% of non-competed FcγRIIb signal in presence of 10x FcγRIIa
⁴IIb-Specific Comparator: Mimoto, et al., 2013, Protein Eng. Des. Sel., 26: 589-598
⁵ND—signal too low for accurate measurement

TABLE 6.19
Strategy 3 Variants

| | | | | | | | | | | IIb | |
|---|---|---|---|---|---|---|---|---|---|---|---|
| | | | | | | IIb-Fold | | IIaR- | | Selectivity² | |
| | Variant | | FcγRIIb | FcγRIIa | IIb- | wrt | IIaR- | Fold wrt | IIb | Fold wrt | |
| Strategy | # | Mutations¹ | K_{D} | R K_{D} | Fold | Control | Fold | Control | Selectivity² | Control | ELISA³ |
| Controls | 16463 | WT | 1.4E−06 | 3.1E−07 | 1.0 | | 1.0 | | 1.0 | | |
| | 27362 | strat3_control | 1.5E−07 | 2.9E−07 | 9.4 | | 1.1 | | 8.7 | | 42.35 |
| | | (A_G236N_G237A | | | | | | | | | |
| | | B_G236D_G237F_{—} | | | | | | | | | |
| | | S239D_S267V_H268D) | | | | | | | | | |
| | 28473 | strat3_control + E269K | 2.0E−07 | 5.1E−07 | 7.1 | 0.8 | 0.6 | 0.6 | 11.9 | 1.4 | 82.49 |
| | v12⁴ | Symmetrical | 1.3E−08 | 2.2E−07 | 111.6 | | 1.4 | | 80.5 | | 100.12 |
| | | E233D_G237D_P238D_H268D_{—} | | | | | | | | | |
| | | P271G_A330R | | | | | | | | | |
| Strat 3 | 27489 | template66_strat3 | 3.9E−08 | 1.3E−07 | 36.8 | — | 2.3 | — | 15.8 | — | 65.34 |
| Unmodified | 27490 | template7_strat3 | 3.2E−08 | 5.8E−08 | 44.9 | — | 5.3 | — | 8.4 | — | 26.07 |
| Loop | 27491 | template151_strat3 | 2.5E−08 | 6.1E−08 | 57.4 | — | 5.1 | — | 11.3 | — | 41.35 |
| Templates | 27492 | template19_strat3 | 3.1E−08 | 4.4E−08 | 45.9 | — | 7.0 | — | 6.5 | — | 15.68 |
| Strat3 | 27363 | template1_T326*H_{—} | 7.7E−09 | 2.8E−08 | 188.4 | 20.0 | 10.9 | 10.1 | 17.2 | 2.0 | 67.73 |
| Loop | | W327*W_F328*S_D329*D_strat3 | | | | | | | | | |
| Template 1 | 27364 | template1_T326*H_{—} | 1.3E−08 | 3.5E−08 | 114.4 | 12.1 | 8.8 | 8.1 | 13.0 | 1.5 | 48.43 |
| | | W327*W_F328*E_D329*D_strat3 | | | | | | | | | |
| | 27365 | template1_T326*H_{—} | 1.7E−09 | 6.9E−09 | 838.3 | 88.9 | 44.8 | 41.5 | 18.7 | 2.1 | 66.21 |
| | | W327*W_F328*E_D329*G_strat3 | | | | | | | | | |
| | 27366 | template1_T326*H_{—} | 3.2E−09 | 1.4E−08 | 450.1 | 47.7 | 22.5 | 20.9 | 20.0 | 2.3 | 72.33 |
| | | W327*W_F328*Q_D329*G_strat3 | | | | | | | | | |
| | 27367 | template1_T326*H_{—} | 9.7E−09 | 3.6E−08 | 148.7 | 15.8 | 8.7 | 8.0 | 17.2 | 2.0 | 64.25 |
| | | W327*W_F328*N_D329*D_strat3 | | | | | | | | | |
| | 27368 | template1_T326*H_{—} | 7.7E−09 | 2.3E−08 | 187.9 | 19.9 | 13.2 | 12.2 | 14.3 | 1.6 | 55.26 |
| | | W327*W_F328*Q_D329*D_strat3 | | | | | | | | | |
| | 27369 | template1_T326*T_{—} | 3.7E−09 | 1.6E−08 | 386.3 | 41.0 | 18.7 | 17.4 | 20.6 | 2.4 | 66.47 |
| | | W327**W_F328*Q_D329*G_strat3 | | | | | | | | | |
| | 27370 | template1_T326*T_{—} | 4.9E−09 | 1.6E−08 | 296.5 | 31.5 | 19.4 | 18.0 | 15.3 | 1.8 | 58.98 |
| | | W327*W_F328*S_D329*G_strat3 | | | | | | | | | |
| | 27371 | template1_T326*H_{—} | 6.1E−09 | 1.8E−08 | 237.0 | 25.1 | 16.7 | 15.5 | 14.2 | 1.6 | 59.73 |
| | | W327*W_F328*T_D329*D_strat3 | | | | | | | | | |
| | 27372 | template1_T326*H_{—} | 1.6E−08 | 4.8E−08 | 92.2 | 9.8 | 6.4 | 5.9 | 14.4 | 1.6 | 53.96 |
| | | W327*W_F328*D_D329*D_strat3 | | | | | | | | | |
| | 27373 | template1_T326*H_{—} | 4.4E−09 | 1.8E−08 | 332.1 | 35.2 | 17.4 | 16.1 | 19.1 | 2.2 | 71.42 |
| | | W327*W_F328*H_D329*D_strat3 | | | | | | | | | |
| | 27374 | template1_S325*A_A331*BN_strat3 | 4.4E−09 | 1.5E−08 | 332.1 | 35.2 | 21.2 | 19.7 | 15.7 | 1.8 | 55.48 |
| | 27375 | template1_T326*H_W327*W_{—} | 8.8E−09 | 3.0E−08 | 164.8 | 17.5 | 10.2 | 9.4 | 16.2 | 1.9 | 72.10 |
| | | F328*S_D329*D_S325*A_strat3 | | | | | | | | | |
| | 27376 | template1_T326*H_W327*W_{—} | ND⁵ | ND | — | — | — | — | — | — | |
| | | F328*E_D329*D_S325*A_strat3 | | | | | | | | | |
| | 27377 | template1_T326*H_W327*W_{—} | 4.4E−09 | 1.0E−08 | 327*.6 | 34.8 | 30.8 | 28.5 | 10,6 | 1.2 | 67.97 |
| | | F328*E_D329*G_S325*A_strat3 | | | | | | | | | |
| | 27378 | template1_T326*H_W327*W_{—} | 4.5E−09 | 1.6E−08 | 322.7 | 34.2 | 19.7 | 18.3 | 16.4 | 1.9 | 66.32 |
| | | F328*Q_D329*G_S325*A_strat3 | | | | | | | | | |
| | 27379 | template1_T326*H_W327*W_{—} | 3.0E−09 | 9.6E−09 | 486.3 | 51.6 | 32.0 | 29.7 | 15.2 | 1.7 | 67.59 |
| | | F328*F_D329*D_S325*A_strat3 | | | | | | | | | |
| | 27380 | template1_T326*T_W327*W_{—} | ND | ND | — | — | — | — | — | — | 54.85 |
| | | F328*S_D329*D_S325*A_strat3 | | | | | | | | | |
| | 27381 | template1_T326*H_W327*W_{—} | 8.4E−09 | 2.5E−08 | 172.3 | 18.3 | 12.2 | 11.3 | 14.2 | 1.6 | |
| | | F328*S_D329*D_A331*BN_strat3 | | | | | | | | | |
| | 27382 | template1_T326*H_W327*W_{—} | 1.9E−08 | 4.0E−08 | 76.9 | 8.2 | 7.7 | 7.1 | 10.0 | 1.1 | |
| | | F328*E_D329*D_A331*BN_strat3 | | | | | | | | | |
| | 27383 | template1_T326*H_W327*W_{—} | 2.4E−09 | 1.1E−08 | 608.5 | 64.5 | 28.1 | 26.0 | 21.7 | 2.5 | |
| | | F328*E_D329*G_A331*BN_strat3 | | | | | | | | | |
| | 27384 | template1_T326*H_W327*W_{—} | 5.6E−09 | 2.0E−08 | 255.9 | 27.1 | 15.5 | 14.4 | 16.5 | 1.9 | 69.15 |
| | | F328*Q_D329*G_A331*BN_strat3 | | | | | | | | | |
| | 27385 | template1_T326*H_W327*W_{—} | 4.3E−09 | 1.5E−08 | 337.3 | 35.8 | 20.9 | 19.4 | 16.1 | 1.8 | 63.18 |
| | | F328*F_D329*D_A331*BN_strat3 | | | | | | | | | |
| | 27386 | template1_T326*T_W327*W_{—} | 1.0E−08 | 3.3E−08 | 138.2 | 14.7 | 9.3 | 8.6 | 14.8 | 1.7 | 45.96 |
| | | F328*S_D329*D_A331*BN_strat3 | | | | | | | | | |
| | 27387 | template1_T326*H_{—} | 1.3E−08 | 3.8E−08 | 108.4 | 11.5 | 8.1 | 7.5 | 13.4 | 1.5 | 59.62 |
| | | W327*W_F328*S_{—} | | | | | | | | | |
| | | D329*D_S325*A_A331*BN_strat3 | | | | | | | | | |
| | 27388 | template1_T326*H_{—} | 1.7E−08 | 3.4E−08 | 87.3 | 9.3 | 9.1 | 8.4 | 9.6 | 1.1 | 32.98 |
| | | W327*W_F328*E_{—} | | | | | | | | | |
| | | D329*D_S325*A_A331*BN_strat3 | | | | | | | | | |
| | 27389 | template1_T326*H_{—} | 2.9E−09 | 1.1E−08 | 505.1 | 53.6 | 28.9 | 26.7 | 17.5 | 2.0 | |
| | | W327*W_F328*E_{—} | | | | | | | | | |
| | | D329*G_S325*A_A331*BN_strat3 | | | | | | | | | |
| | 27390 | template1_T326*H_{—} | 5.8E−09 | 1.9E−08 | 247.9 | 26.3 | 16.0 | 14.8 | 15.5 | 1.8 | 66.17 |
| | | W327*W_F328*Q_{—} | | | | | | | | | |
| | | D329*G_S325*A_A331*BN_strat3 | | | | | | | | | |
| | 27391 | template1_T326*H_W327*W_{—} | 3.6E−09 | 1.2E−08 | 402.4 | 42.7 | 26.8 | 24.8 | 15.0 | 1.7 | 59.48 |
| | | F328*F_D329*D_S325*A_{—} | | | | | | | | | |
| | | A331*BN_strat3 | | | | | | | | | |
| | 27392 | template1_T326*T_{—} | 1.9E−08 | 5.6E−08 | 74.5 | 7.9 | 5.5 | 5.1 | 13.5 | 1.5 | 43.27 |
| | | W327*W_F328*S_{—} | | | | | | | | | |
| | | D329*D_S325*A_A331*BN_strat3 | | | | | | | | | |
| | 27393 | template1_T326*H_{—} | 3.7E−09 | 1.4E−08 | 393.0 | 41.7 | 21.3 | 19.8 | 18.4 | 2.1 | 61.32 |
| | | W327*W_F328*H_D329*G_strat3 | | | | | | | | | |
| | 27394 | template1_T326*T_{—} | 3.4E−09 | 1.4E−08 | 429.6 | 45.6 | 22.6 | 20.9 | 19.0 | 2.2 | 58.36 |
| | | W327*W_F328*H_D329*G_strat3 | | | | | | | | | |
| | 27395 | template1_T326*H_{—} | 8.8E−08 | 1.1E−07 | 16.4 | 1.7 | 2.7 | 2.5 | 6.1 | 0.7 | 16.46 |
| | | W327*D_F328*T_D329*G_strat3 | | | | | | | | | |
| | 27396 | template1_T326*H_{—} | 1.3E−07 | 1.3E−07 | 11.1 | 1.2 | 2.4 | 2.2 | 4.6 | 0.5 | 953.88 |
| | | F328*H_D329*G_W327*T_strat3 | | | | | | | | | |
| | 27397 | template1_T326*T_{—} | 9.2E−08 | 1.1E−07 | 15.8 | 1.7 | 2.7 | 2.5 | 5.9 | 0.7 | |
| | | F328*H_D329*G_W327*T_strat3 | | | | | | | | | |
| | 27398 | template1_T326*H_F328*T_D329*G_{—} | 9.7E−08 | 1.4E−07 | 14.9 | 1.6 | 2.1 | 2.0 | 6.9 | 0.8 | 16.74 |
| | | W327*T_strat3 | | | | | | | | | |
| Strat3 | 27399 | template66_D327*D_{—} | 2.6E−08 | 5.4E−08 | 54.8 | 5.8 | 5.8 | 5.3 | 9.5 | 1.1 | 47.88 |
| Loop | | Q328*D_N329*E_Q330*D_strat3 | | | | | | | | | |
| Template 66 | 27400 | template66_D327*D_{—} | 2.6E−08 | 6.5E−08 | 56.4 | 6.0 | 4.8 | 4.4 | 11.9 | 1.4 | 56.93 |
| | | Q328*P_N329*D_Q330*Q_strat3 | | | | | | | | | |
| | 27401 | template66_D327*D_{—} | 2.5E−08 | 7.4E−08 | 58.2 | 6.2 | 4.2 | 3.9 | 13.9 | 1.6 | 58.02 |
| | | Q328*E_N329*D_Q330*D_strat3 | | | | | | | | | |
| | 27402 | template66_D327*D_{—} | 3.2E−08 | 7.7E−08 | 44.5 | 4.7 | 4.0 | 3.7 | 11.1 | 1.3 | 50.25 |
| | | Q328*E_N329*E_Q330*Q_strat3 | | | | | | | | | |
| | 27403 | template66_D327*D_{—} | 3.0E−08 | 1.0E−07 | 47.8 | 5.1 | 3.0 | 2.7 | 16.1 | 1.8 | 72.73 |
| | | Q328*H_N329*D_Q330*Q_strat3 | | | | | | | | | |
| | 27404 | template66_D327*D_{—} | 2.5E−08 | 9.5E−08 | 57.6 | 6.1 | 3.3 | 3.0 | 17.7 | 2.0 | 75.66 |
| | | Q328*S_N329*T_Q330*D_strat3 | | | | | | | | | |
| | 27405 | template66_D327*D_{—} | 1.9E−08 | 7.0E−08 | 75.3 | 8.0 | 4.4 | 4.1 | 17.2 | 2.0 | 72.04 |
| | | Q328*N_N329*D_Q330*D_strat3 | | | | | | | | | |
| | 27406 | template66_D327*D_{—} | 2.2E−08 | 8.3E−08 | 64.9 | 6.9 | 3.7 | 3.4 | 17.5 | 2.0 | 68.50 |
| | | Q328*T_N329*D_Q330*D_strat3 | | | | | | | | | |
| | 27407 | template66_D327*N_{—} | 4.5E−08 | 9.8E−08 | 32.3 | 3.4 | 3.1 | 2.9 | 10.3 | 1.2 | 40.21 |
| | | Q328*D_N329*E_Q330*Q_strat3 | | | | | | | | | |
| | 27408 | template66_D327*D_{—} | 2.8E−08 | 9.8E−08 | 52.5 | 5.6 | 3.2 | 2.9 | 16.6 | 1.9 | 73.19 |
| | | Q328*S_N329*T_Q330*Q_strat3 | | | | | | | | | |
| | 27409 | template66_I332Q_strat3 | 3.9E−08 | 7.1E−08 | 37.5 | 4.0 | 4.3 | 4.0 | 8.6 | 1.0 | 38.88 |
| | 27410 | template66_D327*D_{—} | 3.0E−08 | 1.0E−07 | 48.8 | 5.2 | 2.9 | 2.7 | 16.6 | 1.9 | 67.14 |
| | | Q328*T_N329*S_Q330*Q_strat3 | | | | | | | | | |
| | 27411 | template66_D327*N_{—} | 5.4E−08 | 1.6E−07 | 26.5 | 2.8 | 1.9 | 1.8 | 13.6 | 1.6 | 69.97 |
| | | Q328*H_N329*N_Q330*D_strat3 | | | | | | | | | |
| | 27412 | template66_D327*D_{—} | 3.2E−08 | 8.6E−08 | 45.1 | 4.8 | 3.6 | 3.3 | 12.6 | 1.4 | 61.57 |
| | | Q328*D_N329*S_Q330*Q_strat3 | | | | | | | | | |
| | 27413 | template66_I332W_strat3 | 9.0E−08 | 1.6E−07 | 16.0 | 1.7 | 1.9 | 1.8 | 8.3 | 1.0 | 44.89 |
| | 27414 | template66_D325*A_strat3 | 5.2E−08 | 1.2E−07 | 27.9 | 3.0 | 2.5 | 2.3 | 11.2 | 1.3 | 42.20 |
| | 27415 | template66_I332Q_D325*A_strat3 | 4.2E−08 | 6.6E−08 | 34.8 | 3.7 | 4.7 | 4.3 | 7.5 | 0.9 | 24.26 |
| | 27416 | template66_D327*D_Q328*D_{—} | ND | ND | — | — | — | — | — | — | 26.93 |
| | | N329*E_Q330*D_I332Q_strat3 | | | | | | | | | |
| | 27417 | template66_D327*D_Q328*P_{—} | ND | ND | — | — | — | — | — | — | |
| | | N329*D_Q330*Q_I332Q_strat3 | | | | | | | | | |
| | 27418 | template66_D327*D_Q328*E_{—} | ND | ND | — | — | — | — | — | — | |
| | | N329*D_Q330*D_I332Q_strat3 | | | | | | | | | |
| | 27419 | template66_D327*D_Q328*E_{—} | 4.0E−08 | 7.3E−08 | 35.8 | 3.8 | 4.2 | 3.9 | 8.5 | 1.0 | 24.42 |
| | | N329*E_Q330*Q_I332Q_strat3 | | | | | | | | | |
| | 27420 | template66_D327*D_Q328*H_{—} | 4.1E−08 | 8.0E−08 | 35.1 | 3.7 | 3.8 | 3.6 | 9.1 | 1.0 | 36.69 |
| | | N329*D_Q330*Q_I332Q_strat3 | | | | | | | | | |
| | 27421 | template66_D327*D_Q328*S_{—} | 2.9E−08 | 5.8E−08 | 49.5 | 5.2 | 5.3 | 4.9 | 9.4 | 1.1 | |
| | | N329*T_Q330*D_I332Q_strat3 | | | | | | | | | |
| | 27422 | template66_D327*D_Q328*D_{—} | 7.0E−08 | 1.5E−07 | 20.6 | 2.2 | 2.0 | 1.8 | 10.3 | 1.2 | 38.66 |
| | | N329*E_Q330*D_I332W_strat3 | | | | | | | | | |
| | 27423 | template66_D327*D_Q328*P_{—} | 9.8E−08 | 2.0E−07 | 14.8 | 1.6 | 1.5 | 1.4 | 9.7 | 1.1 | 40.70 |
| | | N329*D_Q330*Q_I332W_strat3 | | | | | | | | | |
| | 27424 | template66_D327*D_Q328*E_{—} | 8.4E−08 | 1.7E−07 | 17.1 | 1.8 | 1.8 | 1.7 | 9.6 | 1.1 | 46.53 |
| | | N329*D_Q330*D_I332W_strat3 | | | | | | | | | |
| | 27425 | template66_D327*D_Q328*E_{—} | 1.1E−07 | 2.3E−07 | 12.8 | 1.4 | 1.4 | 1.3 | 9.4 | 1.1 | 39.31 |
| | | N329*E_Q330*Q_I332W_strat3 | | | | | | | | | |
| | 27426 | template66_D327*D_Q328*H_{—} | 7.7E−08 | 1.7E−07 | 18.7 | 2.0 | 1.8 | 1.7 | 10.3 | 1.2 | 43.46 |
| | | N329*D_Q330*Q_I332W_strat3 | | | | | | | | | |
| | 27427 | template66_D327*D_Q328*S_{—} | 7.7E−08 | 1.4E−07 | 18.9 | 2.0 | 2.1 | 2.0 | 8.8 | 1.0 | 37.12 |
| | | N329*T_Q330*D_I332W_strat3 | | | | | | | | | |
| | 27428 | template66_D327*D_Q328*D_{—} | 3.5E−08 | 6.2E−08 | 41.1 | 4.4 | 5.0 | 4.6 | 8.3 | 0.9 | 24.58 |
| | | N329*E_Q330*D_D325*A_strat3 | | | | | | | | | |
| | 27429 | template66_D327*D_Q328*P_{—} | 3.2E−08 | 6.2E−08 | 45.6 | 4.8 | 4.9 | 4.6 | 9.2 | 1.1 | |
| | | N329*D_Q330*Q_D325*A_strat3 | | | | | | | | | |
| | 27430 | template66_D327*D_Q328*E_{—} | 4.1E−08 | 6.8E−08 | 35.4 | 3.8 | 4.5 | 4.2 | 7.8 | 0.9 | 29.60 |
| | | N329*D_Q330*D_D325*A_strat3 | | | | | | | | | |
| | 27431 | template66_D327*D_Q328*E_{—} | 4.3E−08 | 7.8E−08 | 33.2 | 3.5 | 3.9 | 3.6 | 8.5 | 1.0 | 30.90 |
| | | N329*E_Q330*Q_D325*A_strat3 | | | | | | | | | |
| | 27432 | template66_D327*D_Q328*H_{—} | 6.7E−08 | 1.3E−07 | 21.7 | 2.3 | 2.3 | 2.2 | 9.3 | 1.1 | 40.36 |
| | | N329*D_Q330*Q_D325*A_strat3 | | | | | | | | | |
| | 27433 | template66_D327*D_Q328*S_{—} | 4.3E−08 | 9.2E−08 | 33.4 | 3.5 | 3.4 | 3.1 | 9.9 | 1.1 | 45.79 |
| | | N329*T_Q330*D_D325*A_strat3 | | | | | | | | | |
| | 27434 | template66_D327*D_Q328*D_{—} | 3.0E−08 | 4.7E−08 | 48.2 | 5.1 | 6.6 | 6.1 | 7.3 | 0.8 | 17.28 |
| | | N329*E_Q330*D_I332Q_{—} | | | | | | | | | |
| | | D325*A_strat3 | | | | | | | | | |
| | 27435 | template66_D327*D_{—} | 3.3E−08 | 4.9E−08 | 43.5 | 4.6 | 6.2 | 5.8 | 7.0 | 0.8 | 20.34 |
| | | Q328*P_N329*D_Q330*Q_I332Q_{—} | | | | | | | | | |
| | | D325*A_strat3 | | | | | | | | | |
| | 27436 | template66_D327*D_{—} | 3.1E−08 | 4.7E−08 | 47.0 | 5.0 | 6.6 | 6.1 | 7.1 | 0.8 | 16.78 |
| | | Q328*E_N329*D_Q330*D_I332Q_{—} | | | | | | | | | |
| | | D325*A_strat3 | | | | | | | | | |
| | 27437 | template66_D327*D_{—} | 2.8E−08 | 4.3E−08 | 50.7 | 5.4 | 7.2 | 6.7 | 7.0 | 0.8 | |
| | | Q328*E_N329*E_Q330*Q_I332Q_{—} | | | | | | | | | |
| | | D325*A_strat3 | | | | | | | | | |
| | 27438 | template66_D327*D_{—} | 4.7E−08 | 8.4E−08 | 30.7 | 3.3 | 3.7 | 3.4 | 8.3 | 1.0 | 19.81 |
| | | Q328*H_N329*D_Q330*Q_I332Q_{—} | | | | | | | | | |
| | | D325*A_strat3 | | | | | | | | | |
| | 27439 | template66_D327*D_{—} | 4.2E−08 | 6.3E−08 | 34.5 | 3.7 | 4.9 | 4.6 | 7.0 | 0.8 | 21.12 |
| | | Q328*S_N329*T_Q330*D_I332Q_{—} | | | | | | | | | |
| | | D325*A_strat3 | | | | | | | | | |
| Strat3 | 27440 | template7_E328*E_E329*N_strat3 | 3.5E−08 | 7.5E−08 | 40.8 | 4.3 | 4.1 | 3.8 | 9.9 | 1.1 | 35.42 |
| Loop | 27441 | template7_E328*T_E329*N_strat3 | 3.0E−08 | 7.4E−08 | 49.0 | 5.2 | 4.2 | 3.9 | 11.7 | 1.3 | 44.81 |
| Template 7 | 27442 | template7_E328*H_E329*R_strat3 | 1.9E−07 | 4.4E−07 | 7.5 | 0.8 | 0.7 | 0.7 | 10.7 | 1.2 | 53.77 |
| | 27443 | template7_E328*Q_E329*S_strat3 | 4.3E−08 | 9.7E−08 | 34.0 | 3.6 | 3.2 | 2.9 | 10.7 | 1.2 | 38.58 |
| | 27444 | template7_E328*H_E329*T_strat3 | 6.6E−08 | 1.6E−07 | 22.0 | 2.3 | 2.0 | 1.8 | 11.1 | 1.3 | 42.79 |
| | 27445 | template7_A331*BV_strat3 | 3.6E−08 | 5.8E−08 | 40.3 | 4.3 | 5.3 | 4.9 | 7.5 | 0.9 | 26.00 |
| | 27446 | template7_A331*BY_strat3 | 1.8E−08 | 4.7E−08 | 82.5 | 8.8 | 6.6 | 6.1 | 12.5 | 1.4 | 45.89 |
| | 27447 | template7_G325*F_strat3 | 2.3E−08 | 4.8E−08 | 62.5 | 6.6 | 6.4 | 5.9 | 9.8 | 1.1 | 34.37 |
| | 27448 | template7_A331*BV_G325*F_strat3 | 2.3E−08 | 6.4E−08 | 62.1 | 6.6 | 4.8 | 4.5 | 12.9 | 1.5 | 52.46 |
| | 27449 | template7_E328*E_{—} | 3.4E−08 | 6.9E−08 | 42.2 | 4.5 | 4.5 | 4.1 | 9.4 | 1.1 | 37.61 |
| | | E329*N_A331*BV_strat3 | | | | | | | | | |
| | 27450 | template7_E328*T_{—} | 2.9E−08 | 4.8E−08 | 49.7 | 5.3 | 6.4 | 6.0 | 7.7 | 0.9 | 27.89 |
| | | E329*N_A331*BV_strat3 | | | | | | | | | |
| | 27451 | template7_E328*H_{—} | 1.2E−07 | 2.6E−07 | 12.4 | 1.3 | 1.2 | 1.1 | 10.3 | 1.2 | 41.97 |
| | | E329*R_A331*BV_strat3 | | | | | | | | | |
| | 27452 | template7_E328*Q_{—} | 3.0E−08 | 5.9E−08 | 48.6 | 5.2 | 5.2 | 4.8 | 9.3 | 1.1 | 28.98 |
| | | E329*S_A331*BV_strat3 | | | | | | | | | |
| | 27453 | template7_E328*E_{—} | 1.3E−08 | 4.4E−08 | 107.9 | 11.4 | 7.1 | 6.6 | 15.3 | 1.7 | 60.81 |
| | | E329*N_A331*BY_strat3 | | | | | | | | | |
| | 27454 | template7_E328*T_{—} | 2.6E−08 | 7.7E−08 | 55.6 | 5.9 | 4.0 | 3.7 | 13.9 | 1.6 | 59.99 |
| | | E329*N_A331*BY_strat3 | | | | | | | | | |
| | 27455 | template7_E328*H_{—} | 1.7E−08 | 1.1E−07 | 86.7 | 9.2 | 2.8 | 2.6 | 30.5 | 3.5 | 91.48 |
| | | E329*R_A331*BY_strat3 | | | | | | | | | |
| | 27456 | template7_E328*Q_{—} | 2.4E−08 | 7.8E−08 | 59.7 | 6.3 | 4.0 | 3.7 | 15.0 | 1.7 | 61.15 |
| | | E329*S_A331*BY_strat3 | | | | | | | | | |
| | 27457 | template7_E328*E_{—} | 2.8E−08 | 5.6E−08 | 52.0 | 5.5 | 5.6 | 5.1 | 9.4 | 1.1 | 43.08 |
| | | E329*N_G325*F_strat3 | | | | | | | | | |
| | 27458 | template7_E328*T_E329*N_G325*F_{—} | 2.9E−08 | 6.2E−08 | 50.2 | 5.3 | 5.0 | 4.6 | 10.1 | 1.2 | 39.05 |
| | | strat3 | | | | | | | | | |
| | 27459 | template7_E328*H_E329*R_G325*F_{—} | 3.4E−08 | 8.3E−08 | 42.1 | 4.5 | 3.7 | 3.4 | 11.3 | 1.3 | 48.07 |
| | | strat3 | | | | | | | | | |
| | 27460 | template7_E328*Q_{—} | 2.6E−08 | 6.1E−08 | 54.8 | 5.8 | 5.1 | 4.7 | 10.7 | 1.2 | 39.00 |
| | | E329*S_G325*F_strat3 | | | | | | | | | |
| | 27461 | template7_E328*E_{—} | 2.3E−08 | 7.0E−08 | 63.5 | 6.7 | 4.4 | 4.1 | 14.4 | 1.6 | 45.46 |
| | | E329*N_A331*BV_G325*F_strat3 | | | | | | | | | |
| | 27462 | template7_E328*T_{—} | 2.6E−08 | 7.3E−08 | 55.8 | 5.9 | 4.2 | 3.9 | 13.1 | 1.5 | 60.07 |
| | | E329*N_A331*BV_G325*F_strat3 | | | | | | | | | |
| | 27463 | template7_E328*H_{—} | 4.1E−08 | 1.2E−07 | 34.9 | 3.7 | 2.6 | 2.4 | 13.5 | 1.5 | 62.40 |
| | | E329*R_A331*BV_G325*F_strat3 | | | | | | | | | |
| | 27464 | template7_E328*Q_{—} | 2.6E−08 | 7.2E−08 | 56.4 | 6.0 | 4.3 | 4.0 | 13.2 | 1.5 | 56.19 |
| | | E329*S_A331*BV_G325*F_strat3 | | | | | | | | | |
| Strat3 | 27466 | template151_Y331*BI_strat3 | 1.1E−08 | 3.6E−08 | 128.8 | 13.7 | 8.6 | 7.9 | 15.0 | 1.7 | 68.54 |
| Loop | 27467 | template151_R331*S_strat3 | 1.8E−08 | 3.6E−08 | 80.3 | 8.5 | 8.7 | 8.0 | 9.3 | 1.1 | 33.25 |
| Template | 27468 | template151_Y331*BQ_strat3 | 1.2E−08 | 2.2E−08 | 118.9 | 12.6 | 14.2 | 13.2 | 8.4 | 1.0 | 34.27 |
| 151 | 27469 | template151_E328*H_E329*N_strat3 | 7.7E−08 | 2.0E−07 | 18.9 | 2.0 | 1.5 | 1.4 | 12.2 | 1.4 | 59.12 |
| | 27470 | template151_E328*E_E329*D_strat3 | 2.7E−08 | 5.8E−08 | 52.9 | 5.6 | 5.3 | 5.0 | 9.9 | 1.1 | 39.42 |
| | 27471 | template151_E328*H_{—} | 3.3E−08 | 1.4E−07 | 43.9 | 4.7 | 2.3 | 2.1 | 19.4 | 2.2 | 79.91 |
| | | E329*N_Y331*BI_strat3 | | | | | | | | | |
| | 27472 | template151_E328*E_{—} | 1.0E−08 | 3.3E−08 | 144.1 | 15.3 | 9.5 | 8.8 | 15.2 | 1.7 | 79.22 |
| | | E329*D_Y331*BI_strat3 | | | | | | | | | |
| | 27473 | template151_E328*E_{—} | 1.8E−08 | 3.4E−08 | 78.8 | 8.4 | 9.1 | 8.4 | 8.7 | 1.0 | |
| | | E329*D_R331*S_strat3 | | | | | | | | | |
| | 27474 | template151_E328*E_{—} | 8.8E−09 | 3.1E−08 | 163.5 | 17.3 | 10.0 | 9.3 | 16.3 | 1.9 | |
| | | E329*D_Y331*BI_R331*S_strat3 | | | | | | | | | |
| Loop | 27475 | template66_D327*D_Q328*D_{—} | 4.2E−08 | 5.2E−08 | 34.5 | 3.7 | 5.9 | 5.5 | 5.8 | 0.7 | 11.63 |
| Template 66- | | N329*E_Q330*D_strat3-HF | | | | | | | | | |
| strat3-HF⁶ | 27476 | template66_D327*D_Q328*P_{—} | 5.0E−08 | 6.6E−08 | 28.6 | 3.0 | 4.7 | 4.3 | 6.1 | 0.7 | 14.39 |
| | | N329*D_Q330*Q_strat3-HF | | | | | | | | | |
| | 27477 | template66_I332Q_strat3-HF | 5.1E−08 | 8.1E−08 | 28.6 | 3.0 | 3.8 | 3.5 | 7.5 | 0.9 | 22.40 |
| | 27478 | template66_D325*A_strat3-HF | 5.9E−08 | 9.7E−08 | 24.4 | 2.6 | 3.2 | 2.9 | 7.7 | 0.9 | 30.14 |
| | 27479 | template66_I332Q_D325*A_{—} | 4.3E−08 | 5.0E−08 | 33.6 | 3.6 | 6.2 | 5.7 | 5.5 | 0.6 | 11.74 |
| | | strat3-HF | | | | | | | | | |
| | 27480 | template66_D327*D_Q328*D_{—} | ND | ND | | | | | | | 17.43 |
| | | N329*E_Q330*D_I332Q_strat3-HF | | | | | | | | | |
| | 27481 | template66_D327*D_Q328*P_{—} | 4.9E−08 | 6.8E−08 | 29.3 | 3.1 | 4.5 | 4.2 | 6.5 | 0.7 | 14.89 |
| | | N329*D_Q330*Q_I332Q_strat3-HF | | | | | | | | | |
| | 27482 | template66_D327*D_{—} | 2.7E−08 | 3.4E−08 | 54.0 | 5.7 | 9.0 | 8.3 | 6.0 | 0.7 | 11.49 |
| | | Q328*D_N329*E_Q330*D_I332Q_{—} | | | | | | | | | |
| | | D325*A_strat3-HF | | | | | | | | | |
| | 27483 | template66_D327*D_{—} | 3.2E−08 | 3.9E−08 | 45.1 | 4.8 | 7.9 | 7.3 | 5.7 | 0.7 | 11.54 |
| | | Q328*P_N329*D_Q330*Q_I332Q_{—} | | | | | | | | | |
| | | D325*A_strat3-HF | | | | | | | | | |
| Loop | 27484 | template7_E328*E_E329*N_{—} | 4.1E−08 | 4.6E−08 | 35.4 | 3.8 | 6.7 | 6.2 | 5.3 | 0.6 | 10.09 |
| Template 7- | | strat3-HF | | | | | | | | | |
| strat3-HF⁷ | 27485 | template7_A331*BV_strat3-HF | 3.6E−08 | 4.4E−08 | 39.6 | 4.2 | 7.0 | 6.5 | 5.7 | 0.6 | 9.70 |
| | 27486 | template7_G325*F_strat3-HF | 1.4E−08 | 2.3E−08 | 103.7 | 11.0 | 13.7 | 12.7 | 7.6 | 0.9 | |
| | 27487 | template7_A331*BV_{—} | 2.0E−08 | 3.6E−08 | 71.9 | 7.6 | 8.6 | 8.0 | 8.4 | 1.0 | 32.92 |
| | | G325*F_strat3-HF | | | | | | | | | |
| | 27488 | template7_E328*E_{—} | 4.5E−08 | 5.5E−08 | 32.4 | 3.4 | 5.6 | 5.2 | 5.8 | 0.7 | 11.92 |
| | | E329*N_A331*BV_strat3-HF | | | | | | | | | |
| Strat3 | 27465 | template19_V325*A_strat3 | 1.9E−08 | 2.1E−08 | 77.1 | 8.2 | 14.9 | 13.8 | 5.2 | 0.6 | 7.51 |
| Loop | 27958 | template19_V325*A_{—} | 1.5E−08 | 1.9E−08 | 97.1 | 0.2 | 16.5 | 0.5 | 5.9 | 0.5 | |
| Template 19 | | E328*D_D329*D | | | | | | | | | |
| | 27959 | template19_V325*A_{—} | 1.9E−08 | 2.1E−08 | 74.7 | 0.2 | 14.7 | 0.4 | 5.1 | 0.4 | 8.70 |
| | | E328*D_D329*E | | | | | | | | | |
| | 27960 | template19_V325*A_{—} | 1.7E−08 | 2.0E−08 | 84.8 | 0.2 | 15.3 | 0.4 | 5.6 | 0.5 | 7.54 |
| | | E328*D_D329*N | | | | | | | | | |
| | 27961 | template19_V325*A_{—} | 1.9E−08 | 2.0E−08 | 74.8 | 0.2 | 15.6 | 0.4 | 4.8 | 0.4 | 6.91 |
| | | E328*D_D329*S | | | | | | | | | |
| | 27962 | template19_V325*A_{—} | 2.3E−08 | 2.6E−08 | 63.4 | 0.2 | 11.8 | 0.3 | 5.4 | 0.5 | 9.40 |
| | | E328*D_D329*H | | | | | | | | | |
| | 27963 | template19_V325*A_{—} | 1.7E−08 | 1.8E−08 | 85.9 | 0.2 | 17.4 | 0.5 | 4.9 | 0.4 | 10.24 |
| | | E328*E_D329*E | | | | | | | | | |
| | 27964 | template19_V325*A_{—} | ND | ND | — | — | — | — | — | — | 8.92 |
| | | E328*E_D329*N | | | | | | | | | |
| | 27965 | template19_V325*A_{—} | 2.4E−08 | 2.4E−08 | 61.3 | 0.1 | 12.8 | 0.4 | 4.8 | 0.4 | 6.39 |
| | | E328*E_D329*S | | | | | | | | | |
| | 27966 | template19_V325*A_{—} | 2.4E−08 | 2.7E−08 | 60.3 | 0.1 | 11.5 | 0.3 | 5.2 | 0.4 | 7.67 |
| | | E328*E_D329*H | | | | | | | | | |
| | 27967 | template19_V325*A_{—} | 3.9E−08 | 3.6E−08 | 36.7 | 0.1 | 8.6 | 0.2 | 4.3 | 0.4 | 8.18 |
| | | E328*N_D329*D | | | | | | | | | |
| | 27968 | template19_V325*A_{—} | 2.0E−08 | 3.1E−08 | 73.5 | 0.2 | 10.1 | 0.3 | 7.3 | 0.6 | 10.85 |
| | | E328*N_D329*E | | | | | | | | | |
| | 27969 | template19_V325*A_{—} | 3.7E−08 | 4.2E−08 | 39.2 | 0.1 | 7.3 | 0.2 | 5.4 | 0.5 | 9.60 |
| | | E328*N_D329*N | | | | | | | | | |
| | 27970 | template19_V325*A_{—} | 4.5E−08 | 4.5E−08 | 32.3 | 0.1 | 6.9 | 0.2 | 4.7 | 0.4 | 7.09 |
| | | E328*N_D329*S | | | | | | | | | |
| | 27971 | template19_V325*A_{—} | 3.1E−08 | 3.4E−08 | 45.9 | 0.1 | 9.0 | 0.3 | 5.1 | 0.4 | |
| | | E328*N_D329*H | | | | | | | | | |
| | 27972 | template19_V325*A_{—} | 3.3E−08 | 3.6E−08 | 44.4 | 0.1 | 8.6 | 0.2 | 5.2 | 0.4 | 7.60 |
| | | E328*S_D329*D | | | | | | | | | |
| | 27973 | template19_V325*A_{—} | 2.5E−08 | 3.5E−08 | 57.6 | 0.1 | 8.8 | 0.3 | 6.5 | 0.6 | 12.50 |
| | | E328*S_D329*E | | | | | | | | | |
| | 27974 | template19_V325*A_{—} | 5.4E−08 | 5.7E−08 | 26.8 | 0.1 | 5.4 | 0.2 | 4.9 | 0.4 | 10.70 |
| | | E328*S_D329*N | | | | | | | | | |
| | 27975 | template19_V325*A_{—} | 5.8E−08 | 4.9E−08 | 24.7 | 0.1 | 6.3 | 0.2 | 4.0 | 0.3 | 7.75 |
| | | E328*S_D329*S | | | | | | | | | |
| | 27976 | template19_V325*A_{—} | 3.8E−08 | 5.0E−08 | 37.8 | 0.1 | 6.1 | 0.2 | 6.2 | 0.5 | 12.12 |
| | | E328*S_D329*H | | | | | | | | | |
| | 27977 | template19_V325*A_{—} | 3.1E−08 | 4.3E−08 | 46.6 | 0.1 | 7.2 | 0.2 | 6.5 | 0.5 | 14.13 |
| | | E328*H_D329*D | | | | | | | | | |
| | 27978 | template19_V325*A_{—} | 1.6E−08 | 2.5E−08 | 92.2 | 0.2 | 12.5 | 0.4 | 7.4 | 0.6 | 23.93 |
| | | E328*H_D329*E | | | | | | | | | |
| | 27979 | template19_V325*A_{—} | 5.5E−08 | 7.0E−08 | 26.1 | 0.1 | 4.4 | 0.1 | 5.9 | 0.5 | 19.05 |
| | | E328*H_D329*N | | | | | | | | | |
| | 27980 | template19_V325*A_{—} | ND | ND | | | | | | | 14.78 |
| | | E328*H_D329*S | | | | | | | | | |
| | 27981 | template19_V325*A_{—} | 4.5E−08 | 7.6E−08 | 32.1 | 0.1 | 4.0 | 0.1 | 7.9 | 0.7 | 22.53 |
| | | E328*H_D329*H_{—} | | | | | | | | | |
| Other IgG4- | 28474 | A_L234F_G236N_H268Q_template1/ | ND | ND | — | — | — | — | — | — | 38.69 |
| Based | | B_G236D_S239D_{—} | | | | | | | | | |
| | | V266L_S267A_H268D | | | | | | | | | |
| | 28476 | A_L234F_G236N_{—} | 2.9E−08 | 6.8E−08 | 49.2 | — | 4.6 | — | 10.8 | — | 49.76 |
| | | K274Q_A327G_A330K_P331S/ | | | | | | | | | |
| | | B_G236D_S239D_{—} | | | | | | | | | |
| | | V266L_S267A_H268D_template1 | | | | | | | | | |

¹Mutation notation is in the format “template1_T326*H_strat3,” where “template1” indicates the parental loop template, “T326*H” indicates the mutation made with “T” representing the parental residue being replaced, 326* representing the position and H representing the replacement residue, and “strat3” specifies the parental CH2 mutations (A_G236N_G237A/B_G236D_G237F_S239D_S267V_H268D)
²Selectivity is defined as IIb-Fold / IIaR-Fold
³% of non-competed FcγRIIb signal in presence of 10× FcγRIIa
⁴IIb-Specific Comparator: Mimoto, et al., 2013, Protein Eng. Des. Sel., 26: 589-598
⁵ND—signal too low for accurate measurement
⁶Loop Template 66 - strat3-HF indicates that the starting loop template was a modified version of Template 66 having the following sequence: DTDQNQGEVT SEQ ID NO: 161]
7Loop Template 7 - strat3-HF indicates that the starting loop template was a modified version of Template 7 having the following sequence: GTDEEGKGAT SEQ ID NO: 143]

TABLE 6.20
Strategy 4 Variants¹

| | | | | | | | | | | IIb | |
|---|---|---|---|---|---|---|---|---|---|---|---|
| | | | | | | IIb-Fold | | IIaR- | | Selectivity³ | |
| | Variant | | FcγRIIb | FcγRIIaR | IIb- | wrt | IIaR- | Fold wrt | IIb | Fold wrt | |
| Strategy | # | Mutations² | K_{D} | K_{D} | Fold | Control | Fold | Control | Selectivity³ | Control | ELISA⁴ |
| Controls | 16463 | WT | 1.4E−06 | 3.1E−07 | 1.0 | | 1.0 | | 1.0 | | |
| | 27362 | strat3_control | 1.5E−07 | 2.9E−07 | 9.4 | 1.0 | 1.1 | 1.0 | 8.7 | 1.0 | 42.35 |
| | | (A_G236N_G237A | | | | | | | | | |
| | | B_G236D_G237F | | | | | | | | | |
| | | S239D_S267V_H268D) | | | | | | | | | |
| | 28473 | strat3_control + E269K | 2.0E−07 | 5.1E−07 | 7.1 | 0.8 | 0.6 | 0.6 | 11.9 | 1.4 | 82.49 |
| | v12⁵ | Symmetrical | 1.3E−08 | 2.2E−07 | 111.6 | | 1.4 | | 80.5 | | 100.12 |
| | | E233D_G237D_P238D_H268D_{—} | | | | | | | | | |
| | | P271G_A330R | | | | | | | | | |
| | 27493 | Template_13_3 | 2.7E−08 | 4.6E−08 | 53.2 | 0.1 | 6.8 | 0.2 | 7.9 | 0.7 | 22.27 |
| Strat4 | 27494 | Template_cl_4372- | ND⁶ | ND | — | — | — | — | — | — | 18.74 |
| Template_13_3 | | 13_3\|L326*L_A331*DA_L331*FL | | | | | | | | | |
| | 27501 | Template_13_3\|L326*T_A331*DA_{—} | 1.4E−08 | 2.1E−08 | 99.9 | 0.2 | 14.9 | 0.4 | 6.7 | 0.6 | 17.83 |
| | | L331*FT | | | | | | | | | |
| | 27505 | Template_13_3\|V266I_L326*T_{—} | 2.0E−08 | 3.6E−08 | 72.8 | 0.2 | 8.5 | 0.2 | 8.5 | 0.7 | 33.29 |
| | | A331*DA_L331*FT | | | | | | | | | |
| | 27506 | Template_13_3\|V266L_L326*T_{—} | 1.7E−08 | 3.4E−08 | 87.1 | 0.2 | 9.1 | 0.3 | 9.5 | 0.8 | 32.00 |
| | | A331*DA_L331*FT | | | | | | | | | |
| | 27507 | Template_13_3\|V266F_L326*T_{—} | 3.3E−07 | 4.4E−07 | 4.4 | 0.0 | 0.7 | 0.0 | 6.2 | 0.5 | 35.33 |
| | | A331*DA_L331*FT | | | | | | | | | |
| | 27508 | Template_13_3\|V273I_L326*T_{—} | 1.1E−08 | 1.6E−08 | 133.8 | 0.3 | 19.8 | 0.6 | 6.8 | 0.6 | 17.44 |
| | | A331*DA_L331*FT | | | | | | | | | |
| | 27509 | Template_13_3\|V273L_L326*T_{—} | 1.3E−08 | 1.7E−08 | 108.7 | 0.3 | 18.5 | 0.5 | 5.9 | 0.5 | 13.33 |
| | | A331*DA_L331*FT | | | | | | | | | |
| | 27510 | Template_13_3\|V273F_L326*T_{—} | 1.8E−08 | 1.9E−08 | 81.2 | 0.2 | 16.3 | 0.5 | 5.0 | 0.4 | 6.90 |
| | | A331*DA_L331*FT | | | | | | | | | |
| | 27511 | Template_13_3\|V325*I_L326*T_{—} | 1.4E−08 | 2.3E−08 | 104.9 | 0.3 | 13.2 | 0.4 | 8.0 | 0.7 | 25.45 |
| | | A331*DA_L331*FT | | | | | | | | | |
| | 27512 | Template_13_3\|V325*L_L326*T_{—} | 6.1E−09 | 1.0E−08 | 236.1 | 0.6 | 29.8 | 0.8 | 7.9 | 0.7 | |
| | | A331*DA_L331*FT | | | | | | | | | |
| | 27513 | Template_13_3\|V325*F_L326*T_{—} | 9.5E−09 | 1.2E−08 | 152.0 | 0.4 | 26.8 | 0.8 | 5.7 | 0.5 | 10.97 |
| | | A331*DA_L331*FT | | | | | | | | | |
| | 27544 | Template_13_3\|V266I_V325*I_{—} | 1.4E−08 | 3.4E−08 | 101.1 | 0.2 | 9.1 | 0.3 | 11.1 | 0.9 | 40.91 |
| | | L326*T_A331*DA_L331*FT | | | | | | | | | |
| | 27545 | Template_13_3\|V266I_V325*L | 7.5E−09 | 1.5E−08 | 192.1 | 0.5 | 20.0 | 0.6 | 9.6 | 0.8 | 18.05 |
| | | L326*T_A331*DA_L331*FT | | | | | | | | | |
| | 27546 | Template_13_3\|V266I_V325*F | 1.0E−08 | 1.5E−08 | 144.7 | 0.3 | 20.2 | 0.6 | 7.2 | 0.6 | 8.70 |
| | | L326*T_A331*DA_L331*FT | | | | | | | | | |
| | 27547 | Template_13_3\|V266L_V325*I_{—} | 1.4E−08 | 3.2E−08 | 101.2 | 0.2 | 9.7 | 0.3 | 10.4 | 0.9 | −0.87 |
| | | L326*T_A331*DA_L331*FT | | | | | | | | | |
| | 27548 | Template_13_3\|V266L_V325*L | 1.1E−08 | 2.1E−08 | 132.1 | 0.3 | 14.8 | 0.4 | 8.9 | 0.8 | 33.38 |
| | | L326*T_A331*DA_L331*FT | | | | | | | | | |
| | 27549 | Template_13_3\|V266L_V325*F_{—} | 1.5E−08 | 2.2E−08 | 98.8 | 0.2 | 14.3 | 0.4 | 6.9 | 0.6 | 12.84 |
| | | L326*T_A331*DA_L331*FT | | | | | | | | | |
| | 27550 | Template_13_3\|V266F_V325*I_{—} | 4.0E−07 | 5.5E−07 | 3.6 | 0.0 | 0.6 | 0.0 | 6.5 | 0.5 | 34.37 |
| | | L326*T_A331*DA_L331*FT | | | | | | | | | |
| | 27551 | Template_13_3\|V266F_V325*L_{—} | 1.3E−07 | 2.0E−07 | 11.0 | 0.0 | 1.6 | 0.0 | 7.0 | 0.6 | 17.96 |
| | | L326*T_A331*DA_L331*FT | | | | | | | | | |
| | 27552 | Template_13_3\|V266F_V325*F_{—} | 1.1E−07 | 1.2E−07 | 13.3 | 0.0 | 2.6 | 0.1 | 5.0 | 0.4 | 8.54 |
| | | L326*T_A331*DA_L331*FT | | | | | | | | | |
| | 27562 | Template_13_3\|V266I_V273I_{—} | 1.2E−08 | 2.0E−08 | 120.3 | 0.3 | 15.2 | 0.4 | 7.9 | 0.7 | 23.33 |
| | | V325*I_L326*T_A331*DA_{—} | | | | | | | | | |
| | | L331*FT | | | | | | | | | |
| | 27563 | Template_13_3\|V266I_V273I_{—} | 8.7E−09 | 1.3E−08 | 166.5 | 0.4 | 23.7 | 0.7 | 7.0 | 0.6 | −0.45 |
| | | V325*L_L326*T_A331*DA_{—} | | | | | | | | | |
| | | L331*FT | | | | | | | | | |
| | 27564 | Template_13_3\|V266I_V273I_{—} | 7.5E−09 | 8.7E−09 | 192.2 | 0.5 | 35.6 | 1.0 | 5.4 | 0.5 | −0.41 |
| | | V325*F_L326*T_A331*DA_{—} | | | | | | | | | |
| | | L331*FT | | | | | | | | | |
| | 27565 | Template_13_3\|V266I_V273L_{—} | 1.2E−08 | 1.7E−08 | 124.8 | 0.3 | 18.3 | 0.5 | 6.8 | 0.6 | 9.15 |
| | | V325*I_L326*T_A331*DA_{—} | | | | | | | | | |
| | | L331*FT | | | | | | | | | |
| | 27566 | Template_13_3\|V266I_V273L_{—} | 9.3E−09 | 1.5E−08 | 155.2 | 0.4 | 20.7 | 0.6 | 7.5 | 0.6 | 23.38 |
| | | V325*L_L326*T_A331*DA_{—} | | | | | | | | | |
| | | L331*FT | | | | | | | | | |
| | 27567 | Template_13_3\|V266I_V273L_{—} | 8.0E−09 | 7.9E−09 | 180.0 | 0.4 | 39.0 | 1.1 | 4.6 | 0.4 | 7.21 |
| | | V325*F_L326*T_A331*DA_{—} | | | | | | | | | |
| | | L331*FT | | | | | | | | | |
| | 27568 | Template_13_3\|V266I_V273F_{—} | 1.1E−08 | 1.1E−08 | 128.8 | 0.3 | 27.4 | 0.8 | 4.7 | 0.4 | 6.52 |
| | | V325*I_L326*T_A331*DA_{—} | | | | | | | | | |
| | | L331*FT | | | | | | | | | |
| | 27569 | Template_13_3\|V266I_V273F_{—} | 9.3E−09 | 1.1E−08 | 155.9 | 0.4 | 28.0 | 0.8 | 5.6 | 0.5 | 7.75 |
| | | V325*L_L326*T_A331*DA_{—} | | | | | | | | | |
| | | L331*FT | | | | | | | | | |
| | 27570 | Template_13_3\|V266I_V273F_{—} | 8.5E−09 | 9.7E−09 | 170.5 | 0.4 | 31.8 | 0.9 | 5.4 | 0.5 | 7.43 |
| | | V325*F_L326*T_A331*DA_{—} | | | | | | | | | |
| | | L331*FT | | | | | | | | | |
| | 27571 | Template_13_3\|V266L_V273I_{—} | 1.8E−08 | 4.1E−08 | 78.4 | 0.2 | 7.4 | 0.2 | 10.5 | 0.9 | 37.81 |
| | | V325*I_L326*T_A331*DA_{—} | | | | | | | | | |
| | | L331*FT | | | | | | | | | |
| | 27572 | Template_13_3\|V266L_V273I_{—} | 1.3E−08 | 3.7E−08 | 111.8 | 0.3 | 8.4 | 0.2 | 13.3 | 1.1 | 163.76 |
| | | V325*L_L326*T_A331*DA_{—} | | | | | | | | | |
| | | L331*FT | | | | | | | | | |
| | 27573 | Template_13_3\|V266L_V273I_{—} | 1.2E−08 | 1.6E−08 | 122.3 | 0.3 | 19.2 | 0.5 | 6.4 | 0.5 | 16.40 |
| | | V325*F_L326*T_A331*DA_{—} | | | | | | | | | |
| | | L331*FT | | | | | | | | | |
| | 27574 | Template_13_3\|V266L_V273L_{—} | 1.9E−08 | 3.5E−08 | 78.1 | 0.2 | 8.8 | 0.2 | 8.9 | 0.8 | 35.07 |
| | | V325*I_L326*T_A331*DA_{—} | | | | | | | | | |
| | | L331*FT | | | | | | | | | |
| | 27575 | Template_13_3\|V266L_V273L_{—} | 1.3E−08 | 2.6E−08 | 110.2 | 0.3 | 12.1 | 0.3 | 9.1 | 0.8 | 40.36 |
| | | V325*L_L326*T_A331*DA_{—} | | | | | | | | | |
| | | L331*FT | | | | | | | | | |
| | 27576 | Template_13_3\|V266L_V273L_{—} | 8.8E−09 | 1.2E−08 | 164.1 | 0.4 | 26.4 | 0.8 | 6.2 | 0.5 | 13.96 |
| | | V325*F_L326*T_A331*DA_{—} | | | | | | | | | |
| | | L331*FT | | | | | | | | | |
| | 27577 | Template_13_3\|V266L_V273F_{—} | 3.6E−08 | 7.0E−08 | 39.9 | 0.1 | 4.4 | 0.1 | 9.0 | 0.8 | 21.55 |
| | | V325*I_L326*T_A331*DA_{—} | | | | | | | | | |
| | | L331*FT | | | | | | | | | |
| | 27578 | Template_13_3\|V266L_V273F_{—} | 2.8E−08 | 6.3E−08 | 52.3 | 0.1 | 4.9 | 0.1 | 10.8 | 0.9 | 39.66 |
| | | V325*L_L326*T_A331*DA_{—} | | | | | | | | | |
| | | L331*FT | | | | | | | | | |
| | 27579 | Template_13_3\|V266L_V273F_{—} | 3.0E−08 | 6.7E−08 | 48.7 | 0.1 | 4.6 | 0.1 | 10.6 | 0.9 | 43.40 |
| | | V325*F_L326*T_A331*DA_{—} | | | | | | | | | |
| | | L331*FT | | | | | | | | | |
| | 27580 | Template_13_3\|V266F_V273I_{—} | ND | ND | — | — | — | — | — | — | 25.04 |
| | | V325*I_L326*T_A331*DA_{—} | | | | | | | | | |
| | | L331*FT | | | | | | | | | |
| | 27581 | Template_13_3\|V266F_V273I_{—} | ND | ND | — | — | — | — | — | — | 15.31 |
| | | V325*L_L326*T_A331*DA_{—} | | | | | | | | | |
| | | L331*FT | | | | | | | | | |
| | 27582 | Template_13_3\|V266F_V273I_{—} | 7.2E−08 | 6.8E−08 | 20.2 | 0.0 | 4.5 | 0.1 | 4.4 | 0.4 | 5.76 |
| | | V325*F_L326*T_A331*DA_{—} | | | | | | | | | |
| | | L331*FT | | | | | | | | | |
| | 27583 | Template_13_3\|V266F_V273L_{—} | 2.3E−07 | 2.9E−07 | 6.4 | 0.0 | 1.1 | 0.0 | 6.0 | 0.5 | 16.64 |
| | | V325*I_L326*T_A331*DA_{—} | | | | | | | | | |
| | | L331*FT | | | | | | | | | |
| | 27584 | Template_13_3\|V266F_V273L_{—} | 1.0E−07 | 1.5E−07 | 14.4 | 0.0 | 2.0 | 0.1 | 7.1 | 0.6 | 14.38 |
| | | V325*L_L326*T_A331*DA_{—} | | | | | | | | | |
| | | L331*FT | | | | | | | | | |
| | 27585 | Template_13_3\|V266F_V273L_{—} | 3.2E−08 | 3.1E−08 | 45.3 | 0.1 | 9.9 | 0.3 | 4.6 | 0.4 | 3.65 |
| | | V325*F_L326*T_A331*DA_{—} | | | | | | | | | |
| | | L331*FT | | | | | | | | | |
| | 27586 | Template_13_3\|V266F_V273F_{—} | ND | ND | — | — | — | — | — | — | 78.78 |
| | | V325*I_L326*T_A331*DA_{—} | | | | | | | | | |
| | | L331*FT | | | | | | | | | |
| | 27587 | Template_13_3\|V266F_V273F_{—} | 5.6E−07 | 7.5E−07 | 2.6 | 0.0 | 0.4 | 0.0 | 6.2 | 0.5 | 78.19 |
| | | V325*L_L326*T_A331*DA_{—} | | | | | | | | | |
| | | L331*FT | | | | | | | | | |
| | 27588 | Template_13_3\|V266F_V273F_{—} | 2.2E−07 | 3.0E−07 | 6.7 | 0.0 | 1.0 | 0.0 | 6.4 | 0.5 | 18.42 |
| | | V325*F_L326*T_A331*DA_{—} | | | | | | | | | |
| | | L331*FT | | | | | | | | | |
| | 27589 | Template_13_3\|V266V_V273V_{—} | 1.2E−08 | 1.8E−08 | 121.1 | 0.3 | 17.4 | 0.5 | 7.0 | 0.6 | 16.32 |
| | | V325*V_L326*T_R331*D_{—} | | | | | | | | | |
| | | E331*AD_A331*DA_L331*FT | | | | | | | | | |
| | 27590 | Template_13_3\|V266V_V273V_{—} | 1.1E−08 | 1.8E−08 | 128.7 | 0.3 | 16.7 | 0.5 | 7.7 | 0.7 | 17.75 |
| | | V325*V_L326*T_R331*D_{—} | | | | | | | | | |
| | | E331*AE_A331*DA_L331*FT | | | | | | | | | |
| | 27591 | Template_13_3\|V266V_V273V_{—} | 1.3E−08 | 1.8E−08 | 108.5 | 0.3 | 17.5 | 0.5 | 6.2 | 0.5 | 16.24 |
| | | V325*V_L326*T_R331*D_{—} | | | | | | | | | |
| | | E331*AS_A331*DA_L331*FT | | | | | | | | | |
| | 27592 | Template_13_3\|V266V_V273V_{—} | 1.2E−08 | 1.8E−08 | 124.3 | 0.3 | 17.1 | 0.5 | 7.3 | 0.6 | 16.00 |
| | | V325*V_L326*T_R331*D_{—} | | | | | | | | | |
| | | E331*AH_A331*DA_L331*FT | | | | | | | | | |
| | 27593 | Template_13_3\|V266V_V273V_{—} | 1.1E−08 | 1.4E−08 | 133.0 | 0.3 | 22.0 | 0.6 | 6.1 | 0.5 | −24.09 |
| | | V325*V_L326*T_R331*D_{—} | | | | | | | | | |
| | | E331*AN_A331*DA_L331*FT | | | | | | | | | |
| | 27594 | Template_13_3\|V266V_V273V_{—} | 1.0E−08 | 1.4E−08 | 143.9 | 0.3 | 21.3 | 0.6 | 6.7 | 0.6 | 17.84 |
| | | V325*V_L326*T_R331*E_{—} | | | | | | | | | |
| | | E331*AD_A331*DA_L331*FT | | | | | | | | | |
| | 27595 | Template_13_3\|V266V_V273V_{—} | 1.1E−08 | 1.6E−08 | 131.1 | 0.3 | 19.4 | 0.6 | 6.7 | 0.6 | 17.67 |
| | | V325*V_L326*T_R331*E_{—} | | | | | | | | | |
| | | E331*AE_A331*DA_L331*FT | | | | | | | | | |
| | 27596 | Template_13_3\|V266V_V273V_{—} | 1.4E−08 | 1.8E−08 | 105.6 | 0.3 | 16.9 | 0.5 | 6.2 | 0.5 | 17.43 |
| | | V325*V_L326*T_R331*E_{—} | | | | | | | | | |
| | | E331*AS_A331*DA_L331*FT | | | | | | | | | |
| | 27597 | Template_13_3\|V266V_V273V_{—} | 1.3E−08 | 1.8E−08 | 113.9 | 0.3 | 16.9 | 0.5 | 6.7 | 0.6 | 16.73 |
| | | V325*V_L326*T_R331*E_{—} | | | | | | | | | |
| | | E331*AH_A331*DA_L331*FT | | | | | | | | | |
| | 27598 | Template_13_3\|V266V_V273V_{—} | 1.2E−08 | 1.7E−08 | 116.9 | 0.3 | 18.1 | 0.5 | 6.5 | 0.5 | 16.22 |
| | | V325*V_L326*T_R331*E_{—} | | | | | | | | | |
| | | E331*AN_A331*DA_L331*FT | | | | | | | | | |
| | 27599 | Template_13_3\|V266V_V273V_{—} | 1.6E−08 | 2.1E−08 | 92.3 | 0.2 | 15.0 | 0.4 | 6.1 | 0.5 | 17.22 |
| | | V325*V_L326*T_R331*S_{—} | | | | | | | | | |
| | | E331*AD_A331*DA_L331*FT | | | | | | | | | |
| | 27600 | Template_13_3\|V266V_V273V_{—} | 1.3E−08 | 1.8E−08 | 113.3 | 0.3 | 17.5 | 0.5 | 6.5 | 0.5 | 11.40 |
| | | V325*V_L326*T_R331*S_{—} | | | | | | | | | |
| | | E331*AE_A331*DA_L331*FT | | | | | | | | | |
| | 27601 | Template_13_3\|V266V_V273V_{—} | 1.2E−08 | 1.7E−08 | 118.7 | 0.3 | 18.2 | 0.5 | 6.5 | 0.6 | 16.18 |
| | | V325*V_L326*T_R331*S_{—} | | | | | | | | | |
| | | E331*AS_A331*DA_L331*FT | | | | | | | | | |
| | 27602 | Template_13_3\|V266V_V273V_{—} | 1.3E−08 | 1.8E−08 | 109.6 | 0.3 | 16.8 | 0.5 | 6.5 | 0.6 | 15.54 |
| | | V325*V_L326*T_R331*S_{—} | | | | | | | | | |
| | | E331*AH_A331*DA_L331*FT | | | | | | | | | |
| | 27603 | Template_13_3\|V266V_V273V_{—} | 1.2E−08 | 1.6E−08 | 118.0 | 0.3 | 19.4 | 0.6 | 6.1 | 0.5 | |
| | | V325*V_L326*T_R331*S_{—} | | | | | | | | | |
| | | E331*AN_A331*DA_L331*FT | | | | | | | | | |
| | 27604 | Template_13_3\|V266V_V273V_{—} | 1.3E−08 | 1.9E−08 | 107.7 | 0.3 | 15.9 | 0.5 | 6.8 | 0.6 | 16.59 |
| | | V325*V_L326*T_R331*H_{—} | | | | | | | | | |
| | | E331*AD_A331*DA_L331*FT | | | | | | | | | |
| | 27605 | Template_13_3\|V266V_V273V_{—} | 1.5E−08 | 2.2E−08 | 94.0 | 0.2 | 14.1 | 0.4 | 6.7 | 0.6 | 17.70 |
| | | V325*V_L326*T_R331*H_{—} | | | | | | | | | |
| | | E331*AE_A331*DA_L331*FT | | | | | | | | | |
| | 27606 | Template_13_3\|V266V_V273V_{—} | 1.4E−08 | 2.1E−08 | 103.7 | 0.2 | 14.6 | 0.4 | 7.1 | 0.6 | 16.93 |
| | | V325*V_L326*T_R331*H_{—} | | | | | | | | | |
| | | E331*AS_A331*DA_L331*FT | | | | | | | | | |
| | 27607 | Template_13_3\|V266V_V273V_{—} | 1.5E−08 | 2.1E−08 | 97.4 | 0.2 | 14.7 | 0.4 | 6.6 | 0.6 | 16.27 |
| | | V325*V_L326*T_R331*H_{—} | | | | | | | | | |
| | | E331*AH_A331*DA_L331*FT | | | | | | | | | |
| | 27608 | Template_13_3\|V266V_V273V_{—} | 1.3E−08 | 1.9E−08 | 112.0 | 0.3 | 16.4 | 0.5 | 6.8 | 0.6 | 16.71 |
| | | V325*V_L326*T_R331*H_{—} | | | | | | | | | |
| | | E331*AN_A331*DA_L331*FT | | | | | | | | | |
| | 27609 | Template_13_3\|V266V_V273V_{—} | 1.2E−08 | 1.7E−08 | 119.3 | 0.3 | 18.4 | 0.5 | 6.5 | 0.5 | 17.45 |
| | | V325*V_L326*T_R331*N_{—} | | | | | | | | | |
| | | E331*AD_A331*DA_L331*FT | | | | | | | | | |
| | 27610 | Template_13_3\|V266V_V273V_{—} | 1.5E−08 | 2.1E−08 | 95.5 | 0.2 | 14.5 | 0.4 | 6.6 | 0.6 | 18.59 |
| | | V325*V_L326*T_R331*N_{—} | | | | | | | | | |
| | | E331*AE_A331*DA_L331*FT | | | | | | | | | |
| | 27611 | Template_13_3\|V266V_V273V_{—} | 1.4E−08 | 2.2E−08 | 104.3 | 0.3 | 14.3 | 0.4 | 7.3 | 0.6 | 17.80 |
| | | V325*V_L326*T_R331*N_{—} | | | | | | | | | |
| | | E331*AS_A331*DA_L331*FT | | | | | | | | | |
| | 27612 | Template_13_3\|V266V_V273V_{—} | 1.5E−08 | 2.2E−08 | 94.3 | 0.2 | 14.2 | 0.4 | 6.6 | 0.6 | 17.72 |
| | | V325*V_L326*T_R331*N_{—} | | | | | | | | | |
| | | E331*AH_A331*DA_L331*FT | | | | | | | | | |
| | 27613 | Template_13_3\|V266V_V273V_{—} | 1.6E−08 | 2.2E−08 | 91.8 | 0.2 | 14.0 | 0.4 | 6.5 | 0.6 | 18.45 |
| | | V325*V_L326*T_R331*N_{—} | | | | | | | | | |
| | | E331*AN_A331*DA_L331*FT | | | | | | | | | |
| | 27614 | Template_13_3\|V266V_V273V_{—} | 1.4E−08 | 2.0E−08 | 105.6 | 0.3 | 15.8 | 0.4 | 6.7 | 0.6 | 18.38 |
| | | V325*V_L326*T_R331*R_{—} | | | | | | | | | |
| | | E331*AD_A331*DA_L331*FT | | | | | | | | | |
| | 27615 | Template_13_3\|V266V_V273V_{—} | 1.5E−08 | 2.1E−08 | 94.6 | 0.2 | 14.4 | 0.4 | 6.6 | 0.6 | 17.27 |
| | | V325*V_L326*T_R331*R_{—} | | | | | | | | | |
| | | E331*AS_A331*DA_L331*FT | | | | | | | | | |
| | 27616 | Template_13_3\|V266V_V273V_{—} | 1.7E−08 | 2.5E−08 | 84.0 | 0.2 | 12.1 | 0.3 | 6.9 | 0.6 | 17.35 |
| | | V325*V_L326*T_R331*R_{—} | | | | | | | | | |
| | | E331*AH_A331*DA_L331*FT | | | | | | | | | |
| | 27617 | Template_13_3\|V266V_V273V_{—} | 1.6E−08 | 2.3E−08 | 88.9 | 0.2 | 13.4 | 0.4 | 6.6 | 0.6 | 18.12 |
| | | V325*V_L326*T_R331*R_{—} | | | | | | | | | |
| | | E331*AN_A331*DA_L331*FT | | | | | | | | | |
| | 27618 | Template_13_3\|V266V_V273V_{—} | 1.3E−08 | 2.2E−08 | 110.7 | 0.3 | 14.2 | 0.4 | 7.8 | 0.7 | 28.08 |
| | | V325*I_L326*T_R331*D_{—} | | | | | | | | | |
| | | E331*AD_A331*DA_L331*FT | | | | | | | | | |
| | 27619 | Template_13_3\|V266V_V273V_{—} | 1.3E−08 | 2.3E−08 | 112.6 | 0.3 | 13.2 | 0.4 | 8.5 | 0.7 | 27.82 |
| | | V325*I_L326*T_R331*D_{—} | | | | | | | | | |
| | | E331*AE_A331*DA_L331*FT | | | | | | | | | |
| | 27620 | Template_13_3\|V266V_V273V_{—} | 1.3E−08 | 2.3E−08 | 108.9 | 0.3 | 13.4 | 0.4 | 8.1 | 0.7 | 26.70 |
| | | V325*I_L326*T_R331*D_{—} | | | | | | | | | |
| | | E331*AS_A331*DA_L331*FT | | | | | | | | | |
| | 27621 | Template_13_3\|V266V_V273V_{—} | 1.6E−08 | 2.5E−08 | 92.5 | 0.2 | 12.3 | 0.4 | 7.5 | 0.6 | 27.07 |
| | | V325*I_L326*T_R331*D_{—} | | | | | | | | | |
| | | E331*AH_A331*DA_L331*FT | | | | | | | | | |
| | 27622 | Template_13_3\|V266V_V273V_{—} | 1.3E−08 | 2.1E−08 | 110.9 | 0.3 | 15.0 | 0.4 | 7.4 | 0.6 | 26.46 |
| | | V325*I_L326*T_R331*D_{—} | | | | | | | | | |
| | | E331*AN_A331*DA_L331*FT | | | | | | | | | |
| | 27623 | Template_13_3\|V266V_V273V_{—} | 1.1E−08 | 1.9E−08 | 135.2 | 0.3 | 15.8 | 0.5 | 8.5 | 0.7 | 27.33 |
| | | V325*I_L326*T_R331*E_{—} | | | | | | | | | |
| | | E331*AD_A331*DA_L331*FT | | | | | | | | | |
| | 27624 | Template_13_3\|V266V_V273V_{—} | 1.1E−08 | 2.0E−08 | 126.8 | 0.3 | 15.2 | 0.4 | 8.3 | 0.7 | 27.06 |
| | | V325*I_L326*T_R331*E_{—} | | | | | | | | | |
| | | E331*AE_A331*DA_L331*FT | | | | | | | | | |
| | 27625 | Template_13_3\|V266V_V273V_{—} | 1.2E−08 | 2.1E−08 | 116.2 | 0.3 | 14.5 | 0.4 | 8.0 | 0.7 | −0.60 |
| | | V325*I_L326*T_R331*E_{—} | | | | | | | | | |
| | | E331*AS_A331*DA_L331*FT | | | | | | | | | |
| | 27626 | Template_13_3\|V266V_V273V_{—} | 1.5E−08 | 2.4E−08 | 96.3 | 0.2 | 13.1 | 0.4 | 7.4 | 0.6 | 26.58 |
| | | V325*I_L326*T_R331*E_{—} | | | | | | | | | |
| | | E331*AH_A331*DA_L331*FT | | | | | | | | | |
| | 27627 | Template_13_3\|V266V_V273V_{—} | 1.5E−08 | 2.4E−08 | 95.7 | 0.2 | 13.1 | 0.4 | 7.3 | 0.6 | 27.81 |
| | | V325*I_L326*T_R331*E_{—} | | | | | | | | | |
| | | E331*AN_A331*DA_L331*FT | | | | | | | | | |
| | 27628 | Template_13_3\|V266V_V273V_{—} | 1.4E−08 | 2.4E−08 | 101.4 | 0.2 | 12.8 | 0.4 | 7.9 | 0.7 | 28.49 |
| | | V325*I_L326*T_R331*S_{—} | | | | | | | | | |
| | | E331*AD_A331*DA_L331*FT | | | | | | | | | |
| | 27629 | Template_13_3\|V266V_V273V_{—} | 1.6E−08 | 2.6E−08 | 89.0 | 0.2 | 11.9 | 0.3 | 7.5 | 0.6 | 28.07 |
| | | V325*I_L326*T_R331*S_{—} | | | | | | | | | |
| | | E331*AE_A331*DA_L331*FT | | | | | | | | | |
| | 27630 | Template_13_3\|V266V_V273V_{—} | 1.4E−08 | 2.3E−08 | 103.8 | 0.2 | 13.4 | 0.4 | 7.8 | 0.7 | 6.98 |
| | | V325*I_L326*T_R331*S_{—} | | | | | | | | | |
| | | E331*AS_A331*DA_L331*FT | | | | | | | | | |
| | 27631 | Template_13_3\|V266V_V273V_{—} | 1.3E−08 | 2.4E−08 | 108.3 | 0.3 | 12.6 | 0.4 | 8.6 | 0.7 | −1.96 |
| | | V325*I_L326*T_R331*S_{—} | | | | | | | | | |
| | | E331*AH_A331*DA_L331*FT | | | | | | | | | |
| | 27632 | Template_13_3\|V266V_V273V_{—} | 1.3E−08 | 2.3E−08 | 109.7 | 0.3 | 13.4 | 0.4 | 8.2 | 0.7 | 24.26 |
| | | V325*I_L326*T_R331*S_{—} | | | | | | | | | |
| | | E331*AN_A331*DA_L331*FT | | | | | | | | | |
| | 27633 | Template_13_3\|V266V_V273V_{—} | 1.2E−08 | 2.3E−08 | 119.8 | 0.3 | 13.7 | 0.4 | 8.7 | 0.7 | 7.51 |
| | | V325*I_L326*T_R331*H_{—} | | | | | | | | | |
| | | E331*AD_A331*DA_L331*FT | | | | | | | | | |
| | 27634 | Template_13_3\|V266V_V273V_{—} | 1.5E−08 | 2.7E−08 | 95.0 | 0.2 | 11.5 | 0.3 | 8.2 | 0.7 | 28.01 |
| | | V325*I_L326*T_R331*H_{—} | | | | | | | | | |
| | | E331*AE_A331*DA_L331*FT | | | | | | | | | |
| | 27635 | Template_13_3\|V266V_V273V_{—} | 1.7E−08 | 2.7E−08 | 84.5 | 0.2 | 11.6 | 0.3 | 7.3 | 0.6 | 26.88 |
| | | V325*I_L326*T_R331*H_{—} | | | | | | | | | |
| | | E331*AS_A331*DA_L331*FT | | | | | | | | | |
| | 27636 | Template_13_3\|V266V_V273V_{—} | 1.6E−08 | 2.8E−08 | 89.4 | 0.2 | 11.2 | 0.3 | 8.0 | 0.7 | 25.17 |
| | | V325*I_L326*T_R331*H_{—} | | | | | | | | | |
| | | E331*AH_A331*DA_L331*FT | | | | | | | | | |
| | 27637 | Template_13_3\|V266V_V273V_{—} | 1.7E−08 | 3.0E−08 | 82.7 | 0.2 | 10.3 | 0.3 | 8.0 | 0.7 | 28.38 |
| | | V325*I_L326*T_R331*H_{—} | | | | | | | | | |
| | | E331*AN_A331*DA_L331*FT | | | | | | | | | |
| | 27638 | Template_13_3\|V266V_V273V_{—} | 1.0E−08 | 2.0E−08 | 138.8 | 0.3 | 15.6 | 0.4 | 8.9 | 0.8 | 26.67 |
| | | V325*I_L326*T_R331*N | | | | | | | | | |
| | | E331*AD_A331*DA_L331*FT | | | | | | | | | |
| | 27639 | Template_13_3\|V266V_V273V_{—} | 1.2E−08 | 2.0E−08 | 120.1 | 0.3 | 15.4 | 0.4 | 7.8 | 0.7 | 28.43 |
| | | V325*I_L326*T_R331*N_{—} | | | | | | | | | |
| | | E331*AE_A331*DA_L331*FT | | | | | | | | | |
| | 27640 | Template_13_3\|V266V_V273V_{—} | 1.3E−08 | 2.2E−08 | 113.9 | 0.3 | 13.8 | 0.4 | 8.3 | 0.7 | 28.19 |
| | | V325*I_L326*T_R331*N_{—} | | | | | | | | | |
| | | E331*AS_A331*DA_L331*FT | | | | | | | | | |
| | 27641 | Template_13_3\|V266V_V273V_{—} | 1.4E−08 | 2.4E−08 | 102.2 | 0.2 | 12.9 | 0.4 | 7.9 | 0.7 | 27.65 |
| | | V325*I_L326*T_R331*N_{—} | | | | | | | | | |
| | | E331*AH_A331*DA_L331*FT | | | | | | | | | |
| | 27642 | Template_13_3\|V266V_V273V_{—} | 1.4E−08 | 2.7E−08 | 106.4 | 0.3 | 11.6 | 0.3 | 9.2 | 0.8 | 27.99 |
| | | V325*I_L326*T_R331*N_{—} | | | | | | | | | |
| | | E331*AN_A331*DA_L331*FT | | | | | | | | | |
| | 27643 | Template_13_3\|V266V_V273V_{—} | 1.7E−08 | 2.5E−08 | 87.3 | 0.2 | 12.2 | 0.3 | 7.1 | 0.6 | 25.49 |
| | | V325*I_L326*T_R331*R_{—} | | | | | | | | | |
| | | E331*AD_A331*DA_L331*FT | | | | | | | | | |
| | 27644 | Template_13_3\|V266V_V273V_{—} | 1.7E−08 | 2.8E−08 | 85.2 | 0.2 | 11.1 | 0.3 | 7.7 | 0.6 | 24.64 |
| | | V325*I_L326*T_R331*R_{—} | | | | | | | | | |
| | | E331*AS_A331*DA_L331*FT | | | | | | | | | |
| | 27645 | Template_13_3\|V266V_V273V_{—} | 2.1E−08 | 3.2E−08 | 68.9 | 0.2 | 9.7 | 0.3 | 7.1 | 0.6 | 24.73 |
| | | V325*I_L326*T_R331*R_{—} | | | | | | | | | |
| | | E331*AH_A331*DA_L331*FT | | | | | | | | | |
| | 27646 | Template_13_3\|V266V_V273V_{—} | 1.5E−08 | 2.5E−08 | 97.8 | 0.2 | 12.3 | 0.4 | 7.9 | 0.7 | 28.48 |
| | | V325*I_L326*T_R331*R_{—} | | | | | | | | | |
| | | E331*AN_A331*DA_L331*FT | | | | | | | | | |
| | 27647 | Template_13_3\|V266I_V273V_{—} | 1.1E−08 | 2.3E−08 | 129.0 | 0.3 | 13.4 | 0.4 | 9.6 | 0.8 | 32.52 |
| | | V325*V_L326*T_R331*D_{—} | | | | | | | | | |
| | | E331*AD_A331*DA_L331*FT | | | | | | | | | |
| | 27648 | Template_13_3\|V266I_V273V_{—} | 1.1E−08 | 2.4E−08 | 126.3 | 0.3 | 13.1 | 0.4 | 9.7 | 0.8 | 38.09 |
| | | V325*V_L326*T_R331*D_{—} | | | | | | | | | |
| | | E331*AE_A331*DA_L331*FT | | | | | | | | | |
| | 27649 | Template_13_3\|V266I_V273V_{—} | 1.3E−08 | 2.5E−08 | 114.9 | 0.3 | 12.2 | 0.3 | 9.4 | 0.8 | 34.99 |
| | | V325*V_L326*T_R331*D_{—} | | | | | | | | | |
| | | E331*AS_A331*DA_L331*FT | | | | | | | | | |
| | 27650 | Template_13_3\|V266I_V273V_{—} | 1.5E−08 | 2.9E−08 | 94.0 | 0.2 | 10.5 | 0.3 | 9.0 | 0.8 | 33.51 |
| | | V325*V_L326*T_R331*D_{—} | | | | | | | | | |
| | | E331*AH_A331*DA_L331*FT | | | | | | | | | |
| | 27651 | Template_13_3\|V266I_V273V_{—} | 1.4E−08 | 2.6E−08 | 103.2 | 0.2 | 11.7 | 0.3 | 8.8 | 0.7 | 37.99 |
| | | V325*V_L326*T_R331*D_{—} | | | | | | | | | |
| | | E331*AN_A331*DA_L331*FT | | | | | | | | | |
| | 27652 | Template_13_3\|V266I_V273V_{—} | 1.3E−08 | 2.5E−08 | 109.6 | 0.3 | 12.3 | 0.4 | 8.9 | 0.8 | 26.49 |
| | | V325*V_L326*T_R331*E_{—} | | | | | | | | | |
| | | E331*AD_A331*DA_L331*FT | | | | | | | | | |
| | 27653 | Template_13_3\|V266I_V273V_{—} | 1.5E−08 | 2.9E−08 | 94.0 | 0.2 | 10.5 | 0.3 | 9.0 | 0.8 | 36.04 |
| | | V325*V_L326*T_R331*E_{—} | | | | | | | | | |
| | | E331*AE_A331*DA_L331*FT | | | | | | | | | |
| | 27654 | Template_13_3\|V266I_V273V_{—} | 1.3E−08 | 2.5E−08 | 115.5 | 0.3 | 12.5 | 0.4 | 9.2 | 0.8 | 34.65 |
| | | V325*V_L326*T_R331*E_{—} | | | | | | | | | |
| | | E331*AS_A331*DA_L331*FT | | | | | | | | | |
| | 27655 | Template_13_3\|V266I_V273V_{—} | 1.3E−08 | 2.5E−08 | 112.4 | 0.3 | 12.3 | 0.3 | 9.2 | 0.8 | 33.21 |
| | | V325*V_L326*T_R331*E_{—} | | | | | | | | | |
| | | E331*AH_A331*DA_L331*FT | | | | | | | | | |
| | 27656 | Template_13_3\|V266I_V273V_{—} | 1.2E−08 | 2.5E−08 | 121.4 | 0.3 | 12.3 | 0.3 | 9.9 | 0.8 | 34.01 |
| | | V325*V_L326*T_R331*E_{—} | | | | | | | | | |
| | | E331*AN_A331*DA_L331*FT | | | | | | | | | |
| | 27657 | Template_13_3\|V266I_V273V_{—} | 1.3E−08 | 2.5E−08 | 110.6 | 0.3 | 12.3 | 0.3 | 9.0 | 0.8 | 34.99 |
| | | V325*V_L326*T_R331*S_{—} | | | | | | | | | |
| | | E331*AD_A331*DA_L331*FT | | | | | | | | | |
| | 27658 | Template_13_3\|V266I_V273V_{—} | 1.6E−08 | 3.2E−08 | 88.2 | 0.2 | 9.8 | 0.3 | 9.0 | 0.8 | 34.33 |
| | | V325*V_L326*T_R331*S_{—} | | | | | | | | | |
| | | E331*AE_A331*DA_L331*FT | | | | | | | | | |
| | 27659 | Template_13_3\|V266I_V273V_{—} | 1.7E−08 | 3.0E−08 | 85.2 | 0.2 | 10.1 | 0.3 | 8.4 | 0.7 | 34.52 |
| | | V325*V_L326*T_R331*S_{—} | | | | | | | | | |
| | | E331*AS_A331*DA_L331*FT | | | | | | | | | |
| | 27660 | Template_13_3\|V266I_V273V_{—} | 1.7E−08 | 3.2E−08 | 87.2 | 0.2 | 9.5 | 0.3 | 9.2 | 0.8 | 32.40 |
| | | V325*V_L326*T_R331*S_{—} | | | | | | | | | |
| | | E331*AH_A331*DA_L331*FT | | | | | | | | | |
| | 27661 | Template_13_3\|V266I_V273V_{—} | 1.8E−08 | 3.1E−08 | 82.0 | 0.2 | 9.9 | 0.3 | 8.3 | 0.7 | 33.05 |
| | | V325*V_L326*T_R331*S_{—} | | | | | | | | | |
| | | E331*AN_A331*DA_L331*FT | | | | | | | | | |
| | 27662 | Template_13_3\|V266I_V273V_{—} | 1.4E−08 | 2.7E−08 | 102.1 | 0.2 | 11.3 | 0.3 | 9.1 | 0.8 | 35.01 |
| | | V325*V_L326*T_R331*H_{—} | | | | | | | | | |
| | | E331*AD_A331*DA_L331*FT | | | | | | | | | |
| | 27663 | Template_13_3\|V266I_V273V_{—} | 1.4E−08 | 2.8E−08 | 106.2 | 0.3 | 11.0 | 0.3 | 9.7 | 0.8 | 34.96 |
| | | V325*V_L326*T_R331*H_{—} | | | | | | | | | |
| | | E331*AE_A331*DA_L331*FT | | | | | | | | | |
| | 27664 | Template_13_3\|V266I_V273V_{—} | 1.5E−08 | 3.0E−08 | 93.8 | 0.2 | 10.4 | 0.3 | 9.0 | 0.8 | 34.68 |
| | | V325*V_L326*T_R331*H_{—} | | | | | | | | | |
| | | E331*AS_A331*DA_L331*FT | | | | | | | | | |
| | 27665 | Template_13_3\|V266I_V273V_{—} | 1.6E−08 | 3.1E−08 | 87.6 | 0.2 | 9.8 | 0.3 | 8.9 | 0.8 | 41.07 |
| | | V325*V_L326*T_R331*H_{—} | | | | | | | | | |
| | | E331*AH_A331*DA_L331*FT | | | | | | | | | |
| | 27666 | Template_13_3\|V266I_V273V_{—} | 1.9E−08 | 3.4E−08 | 74.4 | 0.2 | 9.1 | 0.3 | 8.1 | 0.7 | 36.18 |
| | | V325*V_L326*T_R331*H_{—} | | | | | | | | | |
| | | E331*AN_A331*DA_L331*FT | | | | | | | | | |
| | 27667 | Template_13_3\|V266I_V273V_{—} | 1.4E−08 | 3.0E−08 | 99.9 | 0.2 | 10.4 | 0.3 | 9.6 | 0.8 | 31.59 |
| | | V325*V_L326*T_R331*N_{—} | | | | | | | | | |
| | | E331*AD_A331*DA_L331*FT | | | | | | | | | |
| | 27668 | Template_13_3\|V266I_V273V_{—} | 1.6E−08 | 2.8E−08 | 88.2 | 0.2 | 11.0 | 0.3 | 8.0 | 0.7 | 35.83 |
| | | V325*V_L326*T_R331*N_{—} | | | | | | | | | |
| | | E331*AE_A331*DA_L331*FT | | | | | | | | | |
| | 27669 | Template_13_3\|V266I_V273V_{—} | 1.8E−08 | 3.2E−08 | 82.5 | 0.2 | 9.6 | 0.3 | 8.6 | 0.7 | 33.17 |
| | | V325*V_L326*T_R331*N_{—} | | | | | | | | | |
| | | E331*AS_A331*DA_L331*FT | | | | | | | | | |
| | 27670 | Template_13_3\|V266I_V273V_{—} | 1.5E−08 | 3.0E−08 | 96.2 | 0.2 | 10.4 | 0.3 | 9.2 | 0.8 | 33.92 |
| | | V325*V_L326*T_R331*N_{—} | | | | | | | | | |
| | | E331*AH_A331*DA_L331*FT | | | | | | | | | |
| | 27671 | Template_13_3\|V266I_V273V_{—} | 1.4E−08 | 2.9E−08 | 101.4 | 0.2 | 10.5 | 0.3 | 9.6 | 0.8 | 29.91 |
| | | V325*V_L326*T_R331*N_{—} | | | | | | | | | |
| | | E331*AN_A331*DA_L331*FT | | | | | | | | | |
| | 27672 | Template_13_3\|V266I_V273V_{—} | 1.5E−08 | 2.9E−08 | 99.2 | 0.2 | 10.6 | 0.3 | 9.4 | 0.8 | 32.95 |
| | | V325*V_L326*T_R331*R_{—} | | | | | | | | | |
| | | E331*AD_A331*DA_L331*FT | | | | | | | | | |
| | 27673 | Template_13_3\|V266I_V273V_{—} | 1.8E−08 | 3.4E−08 | 80.5 | 0.2 | 9.0 | 0.3 | 8.9 | 0.8 | 34.85 |
| | | V325*V_L326*T_R331*R_{—} | | | | | | | | | |
| | | E331*AS_A331*DA_L331*FT | | | | | | | | | |
| | 27674 | Template_13_3\|V266I_V273V_{—} | 2.4E−08 | 4.5E−08 | 59.1 | 0.1 | 6.9 | 0.2 | 8.6 | 0.7 | 33.87 |
| | | V325*V_L326*T_R331*R_{—} | | | | | | | | | |
| | | E331*AH_A331*DA_L331*FT | | | | | | | | | |
| | 27675 | Template_13_3\|V266I_V273V_{—} | 1.9E−08 | 3.8E−08 | 75.5 | 0.2 | 8.1 | 0.2 | 9.3 | 0.8 | 34.97 |
| | | V325*V_L326*T_R331*R_{—} | | | | | | | | | |
| | | E331*AN_A331*DA_L331*FT | | | | | | | | | |
| | 27676 | Template_13_3\|V266I_V273V_{—} | 1.3E−08 | 2.9E−08 | 109.3 | 0.3 | 10.6 | 0.3 | 10.4 | 0.9 | 45.18 |
| | | V325*I_L326*T_R331*D_{—} | | | | | | | | | |
| | | E331*AD_A331*DA_L331*FT | | | | | | | | | |
| | 27677 | Template_13_3\|V266I_V273V_{—} | 1.5E−08 | 3.3E−08 | 97.4 | 0.2 | 9.3 | 0.3 | 10.4 | 0.9 | 48.15 |
| | | V325*I_L326*T_R331*D_{—} | | | | | | | | | |
| | | E331*AE_A331*DA_L331*FT | | | | | | | | | |
| | 27678 | Template_13_3\|V266I_V273V_{—} | 1.3E−08 | 3.0E−08 | 110.4 | 0.3 | 10.3 | 0.3 | 10.7 | 0.9 | 49.94 |
| | | V325*I_L326*T_R331*D_{—} | | | | | | | | | |
| | | E331*AS_A331*DA_L331*FT | | | | | | | | | |
| | 27679 | Template_13_3\|V266I_V273V_{—} | 1.2E−08 | 3.0E−08 | 116.8 | 0.3 | 10.4 | 0.3 | 11.2 | 0.9 | 43.37 |
| | | V325*I_L326*T_R331*D_{—} | | | | | | | | | |
| | | E331*AH_A331*DA_L331*FT | | | | | | | | | |
| | 27680 | Template_13_3\|V266I_V273V_{—} | 1.2E−08 | 2.8E−08 | 124.2 | 0.3 | 11.0 | 0.3 | 11.2 | 1.0 | 45.90 |
| | | V325*I_L326*T_R331*D_{—} | | | | | | | | | |
| | | E331*AN_A331*DA_L331*FT | | | | | | | | | |
| | 27681 | Template_13_3\|V266I_V273V_{—} | 1.2E−08 | 2.8E−08 | 119.3 | 0.3 | 11.0 | 0.3 | 10.8 | 0.9 | 46.36 |
| | | V325*I_L326*T_R331*E_{—} | | | | | | | | | |
| | | E331*AD_A331*DA_L331*FT | | | | | | | | | |
| | 27682 | Template_13_3\|V266I_V273V_{—} | 1.5E−08 | 3.3E−08 | 93.5 | 0.2 | 9.4 | 0.3 | 9.9 | 0.8 | 47.41 |
| | | V325*I_L326*T_R331*E_{—} | | | | | | | | | |
| | | E331*AE_A331*DA_L331*FT | | | | | | | | | |
| | 27683 | Template_13_3\|V266I_V273V_{—} | 1.4E−08 | 3.3E−08 | 100.8 | 0.2 | 9.4 | 0.3 | 10.7 | 0.9 | 50.97 |
| | | V325*I_L326*T_R331*E_{—} | | | | | | | | | |
| | | E331*AS_A331*DA_L331*FT | | | | | | | | | |
| | 27684 | Template_13_3\|V266I_V273V_{—} | 1.4E−08 | 3.3E−08 | 103.2 | 0.2 | 9.5 | 0.3 | 10.9 | 0.9 | 46.52 |
| | | V325*I_L326*T_R331*E_{—} | | | | | | | | | |
| | | E331*AH_A331*DA_L331*FT | | | | | | | | | |
| | 27685 | Template_13_3\|V266I_V273V_{—} | 1.6E−08 | 3.4E−08 | 90.5 | 0.2 | 9.0 | 0.3 | 10.0 | 0.8 | 47.63 |
| | | V325*I_L326*T_R331*E_{—} | | | | | | | | | |
| | | E331*AN_A331*DA_L331*FT | | | | | | | | | |
| | 27686 | Template_13_3\|V266I_V273V_{—} | 1.3E−08 | 3.2E−08 | 109.0 | 0.3 | 9.6 | 0.3 | 11.4 | 1.0 | 47.73 |
| | | V325*I_L326*T_R331*S_{—} | | | | | | | | | |
| | | E331*AD_A331*DA_L331*FT | | | | | | | | | |
| | 27687 | Template_13_3\|V266I_V273V_{—} | 1.3E−08 | 3.0E−08 | 109.2 | 0.3 | 10.2 | 0.3 | 10.7 | 0.9 | 40.30 |
| | | V325*I_L326*T_R331*S_{—} | | | | | | | | | |
| | | E331*AE_A331*DA_L331*FT | | | | | | | | | |
| | 27688 | Template_13_3\|V266I_V273V_{—} | 1.4E−08 | 3.3E−08 | 103.0 | 0.2 | 9.5 | 0.3 | 10.9 | 0.9 | 48.12 |
| | | V325*I_L326*T_R331*S_{—} | | | | | | | | | |
| | | E331*AS_A331*DA_L331*FT | | | | | | | | | |
| | 27689 | Template_13_3\|V266I_V273V_{—} | 1.5E−08 | 3.5E−08 | 98.1 | 0.2 | 8.8 | 0.2 | 11.2 | 0.9 | 48.01 |
| | | V325*I_L326*T_R331*S_{—} | | | | | | | | | |
| | | E331*AH_A331*DA_L331*FT | | | | | | | | | |
| | 27690 | Template_13_3\|V266I_V273V_{—} | 1.9E−08 | 3.9E−08 | 78.1 | 0.2 | 8.0 | 0.2 | 9.8 | 0.8 | 46.19 |
| | | V325*I_L326*T_R331*S_{—} | | | | | | | | | |
| | | E331*AN_A331*DA_L331*FT | | | | | | | | | |
| | 27691 | Template_13_3\|V266I_V273V_{—} | 1.5E−08 | 3.3E−08 | 95.4 | 0.2 | 9.4 | 0.3 | 10.1 | 0.9 | 36.12 |
| | | V325*I_L326*T_R331*H_{—} | | | | | | | | | |
| | | E331*AD_A331*DA_L331*FT | | | | | | | | | |
| | 27692 | Template_13_3\|V266I_V273V_{—} | 1.5E−08 | 3.5E−08 | 95.5 | 0.2 | 8.9 | 0.3 | 10.7 | 0.9 | 46.80 |
| | | V325*I_L326*T_R331*H_{—} | | | | | | | | | |
| | | E331*AE_A331*DA_L331*FT | | | | | | | | | |
| | 27693 | Template_13_3\|V266I_V273V_{—} | 1.9E−08 | 4.1E−08 | 77.4 | 0.2 | 7.6 | 0.2 | 10.2 | 0.9 | 43.10 |
| | | V325*I_L326*T_R331*H_{—} | | | | | | | | | |
| | | E331*AS_A331*DA_L331*FT | | | | | | | | | |
| | 27694 | Template_13_3\|V266I_V273V_{—} | 1.6E−08 | 3.6E−08 | 89.5 | 0.2 | 8.5 | 0.2 | 10.5 | 0.9 | 45.29 |
| | | V325*I_L326*T_R331*H_{—} | | | | | | | | | |
| | | E331*AH_A331*DA_L331*FT | | | | | | | | | |
| | 27695 | Template_13_3\|V266I_V273V_{—} | 1.4E−08 | 3.4E−08 | 100.6 | 0.2 | 9.1 | 0.3 | 11.1 | 0.9 | 44.71 |
| | | V325*I_L326*T_R331*H_{—} | | | | | | | | | |
| | | E331*AN_A331*DA_L331*FT | | | | | | | | | |
| | 27696 | Template_13_3\|V266I_V273V_{—} | 1.2E−08 | 2.9E−08 | 120.1 | 0.3 | 10.6 | 0.3 | 11.3 | 1.0 | 48.55 |
| | | V325*I_L326*T_R331*N_{—} | | | | | | | | | |
| | | E331*AD_A331*DA_L331*FT | | | | | | | | | |
| | 27697 | Template_13_3\|V266I_V273V_{—} | 1.3E−08 | 3.1E−08 | 107.3 | 0.3 | 10.1 | 0.3 | 10.6 | 0.9 | 49.36 |
| | | V325*I_L326*T_R331*N_{—} | | | | | | | | | |
| | | E331*AE_A331*DA_L331*FT | | | | | | | | | |
| | 27698 | Template_13_3\|V266I_V273V_{—} | 1.7E−08 | 3.9E−08 | 83.9 | 0.2 | 8.0 | 0.2 | 10.5 | 0.9 | 46.67 |
| | | V325*I_L326*T_R331*N_{—} | | | | | | | | | |
| | | E331*AS_A331*DA_L331*FT | | | | | | | | | |
| | 27699 | Template_13_3\|V266I_V273V_{—} | 1.7E−08 | 4.1E−08 | 82.8 | 0.2 | 7.6 | 0.2 | 10.9 | 0.9 | 43.19 |
| | | V325*I_L326*T_R331*N_{—} | | | | | | | | | |
| | | E331*AH_A331*DA_L331*FT | | | | | | | | | |
| | 27700 | Template_13_3\|V266I_V273V_{—} | 1.4E−08 | 3.2E−08 | 104.4 | 0.3 | 9.5 | 0.3 | 11.0 | 0.9 | 45.74 |
| | | V325*I_L326*T_R331*N_{—} | | | | | | | | | |
| | | E331*AN_A331*DA_L331*FT | | | | | | | | | |
| | 27701 | Template_13_3\|V266I_V273V_{—} | 1.4E−08 | 3.2E−08 | 103.4 | 0.2 | 9.7 | 0.3 | 10.6 | 0.9 | 41.66 |
| | | V325*I_L326*T_R331*R_{—} | | | | | | | | | |
| | | E331*AD_A331*DA_L331*FT | | | | | | | | | |
| | 27702 | Template_13_3\|V266I_V273V_{—} | 1.7E−08 | 3.7E−08 | 87.1 | 0.2 | 8.4 | 0.2 | 10.3 | 0.9 | 42.23 |
| | | V325*I_L326*T_R331*R_{—} | | | | | | | | | |
| | | E331*AS_A331*DA_L331*FT | | | | | | | | | |
| | 27703 | Template_13_3\|V266I_V273V_{—} | 1.8E−08 | 3.9E−08 | 78.4 | 0.2 | 7.9 | 0.2 | 10.0 | 0.8 | 41.00 |
| | | V325*I_L326*T_R331*R_{—} | | | | | | | | | |
| | | E331*AH_A331*DA_L331*FT | | | | | | | | | |
| | 27704 | Template_13_3\|V266I_V273V_{—} | 1.5E−08 | 3.6E−08 | 98.4 | 0.2 | 8.7 | 0.2 | 11.3 | 1.0 | |
| | | V325*I_L326*T_R331*R_{—} | | | | | | | | | |
| | | E331*AN_A331*DA_L331*FT | | | | | | | | | |
| Strat4 | 27514 | Template_12_14\|N325*V_F326*T_{—} | 1.5E−07 | 2.4E−07 | 9.5 | 0.0 | 1.3 | 0.0 | 7.5 | 0.6 | 31.41 |
| Template_12_14 | | F331*CT_I331*ET | | | | | | | | | |
| | 27515 | Template_12_14\|N325*I_F326*T_{—} | 1.1E−07 | 1.6E−07 | 12.9 | 0.0 | 1.9 | 0.1 | 6.8 | 0.6 | 22.13 |
| | | F331*CT_I331*ET | | | | | | | | | |
| | 27516 | Template_12_14\|N325*L_F326*T_{—} | 3.9E−08 | 5.3E−08 | 37.1 | 0.1 | 5.8 | 0.2 | 6.3 | 0.5 | 17.53 |
| | | F331*CT_I331*ET | | | | | | | | | |
| | 27517 | Template_12_14\|N325*F_F326*T_{—} | 7.7E−08 | 8.2E−08 | 18.7 | 0.0 | 3.7 | 0.1 | 5.0 | 0.4 | 9.21 |
| | | F331*CT_I331*ET | | | | | | | | | |
| | 27553 | Template_12_14\|V266I_N325*I_{—} | 1.3E−07 | 2.7E−07 | 10.9 | 0.0 | 1.2 | 0.0 | 9.4 | 0.8 | 39.14 |
| | | F326*T_F331*CT_I331*ET | | | | | | | | | |
| | 27554 | Template_12_14\|V266I_N325*L_{—} | 3.6E−08 | 7.9E−08 | 40.1 | 0.1 | 3.9 | 0.1 | 10.2 | 0.9 | 33.05 |
| | | F326*T_F331*CT_I331*ET | | | | | | | | | |
| | 27555 | Template_12_14\|V266I_N325*F_{—} | 1.0E−07 | 1.3E−07 | 14.2 | 0.0 | 2.4 | 0.1 | 6.0 | 0.5 | 14.54 |
| | | F326*T_F331*CT_I331*ET | | | | | | | | | |
| | 27556 | Template_12_14\|V266L_N325*I_{—} | 1.2E−07 | 2.6E−07 | 12.0 | 0.0 | 1.2 | 0.0 | 10.1 | 0.9 | 43.27 |
| | | F326*T_F331*CT_I331*ET | | | | | | | | | |
| | 27557 | Template_12_14\|V266L_N325*L_{—} | 4.3E−08 | 8.4E−08 | 33.4 | 0.1 | 3.7 | 0.1 | 9.1 | 0.8 | 28.38 |
| | | F326*T_F331*CT_I331*ET | | | | | | | | | |
| | 27558 | Template_12_14\|V266L_N325*F_{—} | 9.5E−08 | 1.6E−07 | 15.2 | 0.0 | 2.0 | 0.1 | 7.8 | 0.7 | 19.03 |
| | | F326*T_F331*CT_I331*ET | | | | | | | | | |
| | 27559 | Template_12_14\|V266F_N325*I_{—} | ND | ND | — | — | — | — | — | — | 2.05 |
| | | F326*T_F331*CT_I331*ET | | | | | | | | | |
| | 27560 | Template_12_14\|V266F_N325*L_{—} | ND | ND | — | — | — | — | — | — | 1037.17 |
| | | F326*T_F331*CT_I331*ET | | | | | | | | | |
| | 27561 | Template_12_14\|V266F_N325*F_{—} | ND | ND | — | — | — | — | — | — | 6.48 |
| | | F326*T_F331*CT_I331*ET | | | | | | | | | |
| | 27705 | Template_12_14\|V266V_V273V_{—} | 3.3E−08 | 5.0E−08 | 43.8 | 0.1 | 6.2 | 0.2 | 7.1 | 0.6 | 24.91 |
| | | N325*I_F326*T_K329*D_{—} | | | | | | | | | |
| | | A330*D_F331*CT_I331*ET | | | | | | | | | |
| | 27706 | Template_12_14\|V266V_V273V_{—} | 3.5E−08 | 4.8E−08 | 41.6 | 0.1 | 6.5 | 0.2 | 6.4 | 0.5 | 23.79 |
| | | N325*I_F326*T_K329*D_{—} | | | | | | | | | |
| | | A330*E_F331*CT_I331*ET | | | | | | | | | |
| | 27707 | Template_12_14\|V266V_V273V_{—} | 4.7E−08 | 6.5E−08 | 30.7 | 0.1 | 4.8 | 0.1 | 6.5 | 0.5 | 23.46 |
| | | N325*I_F326*T_K329*D_{—} | | | | | | | | | |
| | | A330*N_F331*CT_I331*ET | | | | | | | | | |
| | 27708 | Template_12_14\|V266V_V273V_{—} | 3.3E−08 | 4.8E−08 | 44.0 | 0.1 | 6.4 | 0.2 | 6.8 | 0.6 | |
| | | N325*I_F326*T_K329*D_{—} | | | | | | | | | |
| | | A330*S_F331*CT_I331*ET | | | | | | | | | |
| | 27709 | Template_12_14\|V266V_V273V_{—} | 3.1E−08 | 5.1E−08 | 46.4 | 0.1 | 6.0 | 0.2 | 7.7 | 0.7 | 21.16 |
| | | N325*I_F326*T_K329*D_{—} | | | | | | | | | |
| | | A330*H_F331*CT_I331*ET | | | | | | | | | |
| | 27710 | Template_12_14\|V266V_V273V_{—} | 3.0E−08 | 4.6E−08 | 48.9 | 0.1 | 6.7 | 0.2 | 7.3 | 0.6 | 22.07 |
| | | N325*I_F326*T_K329*D_{—} | | | | | | | | | |
| | | A330*A_F331*CT_I331*ET | | | | | | | | | |
| | 27711 | Template_12_14\|V266V_V273V_{—} | 2.6E−08 | 4.3E−08 | 55.5 | 0.1 | 7.2 | 0.2 | 7.7 | 0.6 | 26.36 |
| | | N325*I_F326*T_K329*E_{—} | | | | | | | | | |
| | | A330*D_F331*CT_I331*ET | | | | | | | | | |
| | 27712 | Template_12_14\|V266V_V273V_{—} | 2.4E−08 | 4.6E−08 | 59.9 | 0.1 | 6.6 | 0.2 | 9.0 | 0.8 | 3.58 |
| | | N325*I_F326*T_K329*E_{—} | | | | | | | | | |
| | | A330*E_F331*CT_I331*ET | | | | | | | | | |
| | 27713 | Template_12_14\|V266V_V273V_{—} | 3.7E−08 | 5.7E−08 | 39.2 | 0.1 | 5.4 | 0.2 | 7.2 | 0.6 | 25.21 |
| | | N325*I_F326*T_K329*E_{—} | | | | | | | | | |
| | | A330*N_F331*CT_I331*ET | | | | | | | | | |
| | 27714 | Template_12_14\|V266V_V273V_{—} | 4.0E−08 | 5.9E−08 | 36.4 | 0.1 | 5.2 | 0.1 | 7.0 | 0.6 | 24.78 |
| | | N325*I_F326*T_K329*E_{—} | | | | | | | | | |
| | | A330*S_F331*CT_I331*ET | | | | | | | | | |
| | 27715 | Template_12_14\|V266V_V273V_{—} | 3.9E−08 | 6.0E−08 | 37.0 | 0.1 | 5.2 | 0.1 | 7.1 | 0.6 | 24.51 |
| | | N325*I_F326*T_K329*E_{—} | | | | | | | | | |
| | | A330*H_F331*CT_I331*ET | | | | | | | | | |
| | 27716 | Template_12_14\|V266V_V273V_{—} | 2.6E−08 | 4.4E−08 | 54.9 | 0.1 | 7.1 | 0.2 | 7.8 | 0.7 | 22.78 |
| | | N325*I_F326*T_K329*E_{—} | | | | | | | | | |
| | | A330*A_F331*CT_I331*ET | | | | | | | | | |
| | 27717 | Template_12_14\|V266V_V273V_{—} | 4.3E−08 | 7.1E−08 | 33.4 | 0.1 | 4.4 | 0.1 | 7.6 | 0.6 | 23.34 |
| | | N325*I_F326*T_K329*N_{—} | | | | | | | | | |
| | | A330*D_F331*CT_I331*ET | | | | | | | | | |
| | 27718 | Template_12_14\|V266V_V273V_{—} | 3.9E−08 | 6.2E−08 | 36.7 | 0.1 | 4.9 | 0.1 | 7.4 | 0.6 | 20.74 |
| | | N325*I_F326*T_K329*N_{—} | | | | | | | | | |
| | | A330*E_F331*CT_I331*ET | | | | | | | | | |
| | 27719 | Template_12_14\|V266V_V273V_{—} | 6.3E−08 | 9.7E−08 | 22.9 | 0.1 | 3.2 | 0.1 | 7.2 | 0.6 | 22.55 |
| | | N325*I_F326*T_K329*N_{—} | | | | | | | | | |
| | | A330*N_F331*CT_I331*ET | | | | | | | | | |
| | 27720 | Template_12_14\|V266V_V273V_{—} | 6.7E−08 | 9.3E−08 | 21.6 | 0.1 | 3.3 | 0.1 | 6.5 | 0.5 | 20.58 |
| | | N325*I_F326*T_K329*N_{—} | | | | | | | | | |
| | | A330*S_F331*CT_I331*ET | | | | | | | | | |
| | 27721 | Template_12_14\|V266V_V273V_{—} | 7.8E−08 | 1.1E−07 | 18.4 | 0.0 | 2.7 | 0.1 | 6.7 | 0.6 | 23.41 |
| | | N325*I_F326*T_K329*N_{—} | | | | | | | | | |
| | | A330*H_F331*CT_I331*ET | | | | | | | | | |
| | 27722 | Template_12_14\|V266V_V273V_{—} | 7.4E−08 | 1.1E−07 | 19.5 | 0.0 | 2.8 | 0.1 | 6.9 | 0.6 | 21.84 |
| | | N325*I_F326*T_K329*N_{—} | | | | | | | | | |
| | | A330*A_F331*CT_I331*ET | | | | | | | | | |
| | 27723 | Template_12_14\|V266V_V273V_{—} | 4.8E−08 | 7.1E−08 | 30.4 | 0.1 | 4.3 | 0.1 | 7.0 | 0.6 | 23.80 |
| | | N325*I_F326*T_K329*S_{—} | | | | | | | | | |
| | | A330*D_F331*CT_I331*ET | | | | | | | | | |
| | 27724 | Template_12_14\|V266V_V273V_{—} | 3.4E−08 | 5.4E−08 | 42.0 | 0.1 | 5.7 | 0.2 | 7.4 | 0.6 | 23.20 |
| | | N325*I_F326*T_K329*S_{—} | | | | | | | | | |
| | | A330*E_F331*CT_I331*ET | | | | | | | | | |
| | 27725 | Template_12_14\|V266V_V273V_{—} | 6.0E−08 | 9.5E−08 | 24.0 | 0.1 | 3.2 | 0.1 | 7.4 | 0.6 | 20.32 |
| | | N325*I_F326*T_K329*S_{—} | | | | | | | | | |
| | | A330*N_F331*CT_I331*ET | | | | | | | | | |
| | 27726 | Template_12_14\|V266V_V273V_{—} | ND | ND | — | — | — | — | — | — | |
| | | N325*I_F326*T_K329*S_{—} | | | | | | | | | |
| | | A330*S_F331*CT_I331*ET | | | | | | | | | |
| | 27727 | Template_12_14\|V266V_V273V_{—} | 6.8E−08 | 9.7E−08 | 21.3 | 0.1 | 3.2 | 0.1 | 6.7 | 0.6 | 23.09 |
| | | N325*I_F326*T_K329*S_{—} | | | | | | | | | |
| | | A330*H_F331*CT_I331*ET | | | | | | | | | |
| | 27728 | Template_12_14\|V266V_V273V_{—} | 5.3E−08 | 7.4E−08 | 27.2 | 0.1 | 4.2 | 0.1 | 6.5 | 0.6 | 20.33 |
| | | N325*I_F326*T_K329*S_{—} | | | | | | | | | |
| | | A330*A_F331*CT_I331*ET | | | | | | | | | |
| | 27729 | Template_12_14\|V266V_V273V_{—} | 4.6E−08 | 7.1E−08 | 31.2 | 0.1 | 4.3 | 0.1 | 7.2 | 0.6 | 20.26 |
| | | N325*I_F326*T_K329*H_{—} | | | | | | | | | |
| | | A330*D_F331*CT_I331*ET | | | | | | | | | |
| | 27730 | Template_12_14\|V266V_V273V_{—} | 4.4E−08 | 6.6E−08 | 33.1 | 0.1 | 4.7 | 0.1 | 7.1 | 0.6 | 24.63 |
| | | N325*I_F326*T_K329*H_{—} | | | | | | | | | |
| | | A330*E_F331*CT_I331*ET | | | | | | | | | |
| | 27731 | Template_12_14\|V266V_V273V_{—} | 7.6E−08 | 1.0E−07 | 18.9 | 0.0 | 2.9 | 0.1 | 6.4 | 0.5 | 23.47 |
| | | N325*I_F326*T_K329*H_{—} | | | | | | | | | |
| | | A330*N_F331*CT_I331*ET | | | | | | | | | |
| | 27732 | Template_12_14\|_V266V_V273V_{—} | 7.0E−08 | 9.1E−08 | 20.6 | 0.0 | 3.4 | 0.1 | 6.1 | 0.5 | 19.05 |
| | | N325*I_F326*T_K329*H_{—} | | | | | | | | | |
| | | A330*S_F331*CT_I331*ET | | | | | | | | | |
| | 27733 | Template_12_14\|V266V_V273V_{—} | 6.6E−08 | 1.0E−07 | 21.9 | 0.1 | 3.0 | 0.1 | 7.2 | 0.6 | 21.32 |
| | | N325*I_F326*T_K329*H_{—} | | | | | | | | | |
| | | A330*H_F331*CT_I331*ET | | | | | | | | | |
| | 27734 | Template_12_14\|V266V_V273V_{—} | 5.6E−08 | 8.4E−08 | 25.7 | 0.1 | 3.7 | 0.1 | 7.0 | 0.6 | 19.50 |
| | | N325*I_F326*T_K329*H_{—} | | | | | | | | | |
| | | A330*A_F331*CT_I331*ET | | | | | | | | | |
| | 27735 | Template_12_14\|V266V_V273V_{—} | ND | ND | — | — | — | — | — | — | |
| | | N325*I_F326*T_K329*K_{—} | | | | | | | | | |
| | | A330*D_F331*CT_I331*ET | | | | | | | | | |
| | 27736 | Template_12_14\|V266V_V273V_{—} | 4.8E−08 | 6.5E−08 | 29.8 | 0.1 | 4.7 | 0.1 | 6.3 | 0.5 | 20.78 |
| | | N325*I_F326*T_K329*K_{—} | | | | | | | | | |
| | | A330*E_F331*CT_I331*ET | | | | | | | | | |
| | 27737 | Template_12_14\|V266V_V273V_{—} | 9.2E−08 | 1.3E−07 | 15.7 | 0.0 | 2.3 | 0.1 | 6.8 | 0.6 | 22.80 |
| | | N325*I_F326*T_K329*K_{—} | | | | | | | | | |
| | | A330*N_F331*CT_I331*ET | | | | | | | | | |
| | 27738 | Template_12_14\|V266V_V273V_{—} | 1.4E−07 | 1.9E−07 | 10.2 | 0.0 | 1.7 | 0.0 | 6.2 | 0.5 | 22.45 |
| | | N325*I_F326*T_K329*K_{—} | | | | | | | | | |
| | | A330*S_F331*CT_I331*ET | | | | | | | | | |
| | 27739 | Template_12_14\|V266V_V273V_{—} | 1.0E−07 | 1.4E−07 | 13.9 | 0.0 | 2.3 | 0.1 | 6.2 | 0.5 | 18.98 |
| | | N325*I_F326*T_K329*K_{—} | | | | | | | | | |
| | | A330*H_F331*CT_I331*ET | | | | | | | | | |
| | 27740 | Template_12_14\|V266I_V273V_{—} | 3.0E−08 | 6.4E−08 | 47.6 | 0.1 | 4.8 | 0.1 | 9.9 | 0.8 | 41.70 |
| | | N325*I_F326*T_K329*D_{—} | | | | | | | | | |
| | | A330*D_F331*CT_I331*ET | | | | | | | | | |
| | 27741 | Template_12_14\|V266I_V273V_{—} | 3.1E−08 | 6.4E−08 | 47.3 | 0.1 | 4.8 | 0.1 | 9.8 | 0.8 | 42.62 |
| | | N325*I_F326*T_K329*D_{—} | | | | | | | | | |
| | | A330*E_F331*CT_I331*ET | | | | | | | | | |
| | 27742 | Template_12_14\|V266I_V273V_{—} | 4.5E−08 | 9.4E−08 | 32.3 | 0.1 | 3.3 | 0.1 | 9.8 | 0.8 | 40.34 |
| | | N325*I_F326*T_K329*D_{—} | | | | | | | | | |
| | | A330*N_F331*CT_I331*ET | | | | | | | | | |
| | 27743 | Template_12_14\|V266I_V273V_{—} | 4.3E−08 | 7.8E−08 | 33.2 | 0.1 | 4.0 | 0.1 | 8.4 | 0.7 | |
| | | N325*I_F326*T_K329*D_{—} | | | | | | | | | |
| | | A330*S_F331*CT_I331*ET | | | | | | | | | |
| | 27744 | Template_12_14\|V266I_V273V_{—} | 4.1E−08 | 7.4E−08 | 34.9 | 0.1 | 4.2 | 0.1 | 8.4 | 0.7 | 33.40 |
| | | N325*I_F326*T_K329*D_{—} | | | | | | | | | |
| | | A330*H_F331*CT_I331*ET | | | | | | | | | |
| | 27745 | Template_12_14\|V266I_V273V_{—} | 4.3E−08 | 7.8E−08 | 33.9 | 0.1 | 4.0 | 0.1 | 8.6 | 0.7 | 36.64 |
| | | N325*I_F326*T_K329*D_{—} | | | | | | | | | |
| | | A330*A_F331*CT_I331*ET | | | | | | | | | |
| | 27746 | Template_12_14\|V266I_V273V_{—} | 3.7E−08 | 7.3E−08 | 38.7 | 0.1 | 4.2 | 0.1 | 9.2 | 0.8 | 42.80 |
| | | N325*I_F326*T_K329*E_{—} | | | | | | | | | |
| | | A330*D_F331*CT_I331*ET | | | | | | | | | |
| | 27747 | Template_12_14\|V266I_V273V_{—} | 3.7E−08 | 7.1E−08 | 38.7 | 0.1 | 4.3 | 0.1 | 8.9 | 0.8 | 40.95 |
| | | N325*I_F326*T_K329*E_{—} | | | | | | | | | |
| | | A330*E_F331*CT_I331*ET | | | | | | | | | |
| | 27748 | Template_12_14\|V266I_V273V_{—} | 4.5E−08 | 8.5E−08 | 32.3 | 0.1 | 3.6 | 0.1 | 8.9 | 0.8 | 45.53 |
| | | N325*I_F326*T_K329*E_{—} | | | | | | | | | |
| | | A330*N_F331*CT_I331*ET | | | | | | | | | |
| | 27749 | Template_12_14\|V266I_V273V_{—} | 4.1E−08 | 8.7E−08 | 35.5 | 0.1 | 3.5 | 0.1 | 10.1 | 0.9 | 39.92 |
| | | N325*I_F326*T_K329*E_{—} | | | | | | | | | |
| | | A330*S_F331*CT_I331*ET | | | | | | | | | |
| | 27750 | Template_12_14\|V266I_V273V_{—} | 4.1E−08 | 8.3E−08 | 35.3 | 0.1 | 3.7 | 0.1 | 9.5 | 0.8 | 37.57 |
| | | N325*I_F326*T_K329*E_{—} | | | | | | | | | |
| | | A330*H_F331*CT_I331*ET | | | | | | | | | |
| | 27751 | Template_12_14\|V266I_V273V_{—} | 3.5E−08 | 7.8E−08 | 41.7 | 0.1 | 4.0 | 0.1 | 10.5 | 0.9 | 40.84 |
| | | N325*I_F326*T_K329*E_{—} | | | | | | | | | |
| | | A330*A_F331*CT_I331*ET | | | | | | | | | |
| | 27752 | Template_12_14\|V266I_V273V_{—} | 6.8E−08 | 1.1E−07 | 21.3 | 0.1 | 2.9 | 0.1 | 7.4 | 0.6 | 43.93 |
| | | N325*I_F326*T_K329*N_{—} | | | | | | | | | |
| | | A330*D_F331*CT_I331*ET | | | | | | | | | |
| | 27753 | Template_12_14\|V266I_V273V_{—} | 6.0E−08 | 1.1E−07 | 24.1 | 0.1 | 2.7 | 0.1 | 8.9 | 0.7 | 39.86 |
| | | N325*I_F326*T_K329*N_{—} | | | | | | | | | |
| | | A330*E_F331*CT_I331*ET | | | | | | | | | |
| | 27754 | Template_12_14\|V266I_V273V_{—} | 9.7E−08 | 1.8E−07 | 14.9 | 0.0 | 1.8 | 0.1 | 8.5 | 0.7 | 43.33 |
| | | N325*I_F326*T_K329*N_{—} | | | | | | | | | |
| | | A330*N_F331*CT_I331*ET | | | | | | | | | |
| | 27755 | Template_12_14\|V266I_V273V_{—} | 9.8E−08 | 1.9E−07 | 14.8 | 0.0 | 1.6 | 0.0 | 9.2 | 0.8 | 42.37 |
| | | N325*I_F326*T_K329*N_{—} | | | | | | | | | |
| | | A330*S_F331*CT_I331*ET | | | | | | | | | |
| | 27756 | Template_12_14\|V266I_V273V_{—} | 8.0E−08 | 1.7E−07 | 18.2 | 0.0 | 1.9 | 0.1 | 9.7 | 0.8 | 44.35 |
| | | N325*I_F326*T_K329*N_{—} | | | | | | | | | |
| | | A330*H_F331*CT_I331*ET | | | | | | | | | |
| | 27757 | Template_12_14\|V266I_V273V_{—} | 7.8E−08 | 1.6E−07 | 18.5 | 0.0 | 1.9 | 0.1 | 9.7 | 0.8 | 40.29 |
| | | N325*I_F326*T_K329*N_{—} | | | | | | | | | |
| | | A330*A_F331*CT_I331*ET | | | | | | | | | |
| | 27758 | Template_12_14\|V266I_V273V_{—} | 5.1E−08 | 1.1E−07 | 28.2 | 0.1 | 2.8 | 0.1 | 10.1 | 0.9 | 42.64 |
| | | N325*I_F326*T_K329*S_{—} | | | | | | | | | |
| | | A330*D_F331*CT_I331*ET | | | | | | | | | |
| | 27759 | Template_12_14\|V266I_V273V_{—} | 5.0E−08 | 9.6E−08 | 29.0 | 0.1 | 3.2 | 0.1 | 9.1 | 0.8 | 39.03 |
| | | N325*I_F326*T_K329*S_{—} | | | | | | | | | |
| | | A330*E_F331*CT_I331*ET | | | | | | | | | |
| | 27760 | Template_12_14\|V266I_V273V_{—} | 5.9E−08 | 1.5E−07 | 24.4 | 0.1 | 2.0 | 0.1 | 12.0 | 1.0 | |
| | | N325*I_F326*T_K329*S_{—} | | | | | | | | | |
| | | A330*N_F331*CT_I331*ET | | | | | | | | | |
| | 27761 | Template_12_14\|V266I_V273V_{—} | 7.7E−08 | 1.5E−07 | 18.8 | 0.0 | 2.1 | 0.1 | 9.0 | 0.8 | 39.57 |
| | | N325*I_F326*T_K329*S_{—} | | | | | | | | | |
| | | A330*S_F331*CT_I331*ET | | | | | | | | | |
| | 27762 | Template_12_14\|V266I_V273V_{—} | 8.7E−08 | 1.4E−07 | 16.6 | 0.0 | 2.2 | 0.1 | 7.6 | 0.6 | 192.61 |
| | | N325*I_F326*T_K329*S_{—} | | | | | | | | | |
| | | A330*H_F331*CT_I331*ET | | | | | | | | | |
| | 27763 | Template_12_14\|V266I_V273V_{—} | 7.7E−08 | 1.4E−07 | 18.8 | 0.0 | 2.2 | 0.1 | 8.5 | 0.7 | 44.84 |
| | | N325*I_F326*T_K329*S_{—} | | | | | | | | | |
| | | A330*A_F331*CT_I331*ET | | | | | | | | | |
| | 27764 | Template_12_14\|V266I_V273V_{—} | 5.6E−08 | 1.1E−07 | 25.6 | 0.1 | 2.8 | 0.1 | 9.1 | 0.8 | 42.07 |
| | | N325*I_F326*T_K329*H_{—} | | | | | | | | | |
| | | A330*D_F331*CT_I331*ET | | | | | | | | | |
| | 27765 | Template_12_14\|V266I_V273V_{—} | 4.7E−08 | 9.7E−08 | 30.6 | 0.1 | 3.2 | 0.1 | 9.6 | 0.8 | 44.21 |
| | | N325*I_F326*T_K329*H_{—} | | | | | | | | | |
| | | A330*E_F331*CT_I331*ET | | | | | | | | | |
| | 27766 | Template_12_14\|V266I_V273V_{—} | 7.9E−08 | 1.5E−07 | 18.4 | 0.0 | 2.0 | 0.1 | 9.1 | 0.8 | 34.73 |
| | | N325*I_F326*T_K329*H_{—} | | | | | | | | | |
| | | A330*N_F331*CT_I331*ET | | | | | | | | | |
| | 27767 | Template_12_14\|V266I_V273V_{—} | 8.4E−08 | 1.6E−07 | 17.1 | 0.0 | 2.0 | 0.1 | 8.7 | 0.7 | 31.23 |
| | | N325*I_F326*T_K329*H_{—} | | | | | | | | | |
| | | A330*S_F331*CT_I331*ET | | | | | | | | | |
| | 27768 | Template_12_14\|V266I_V273V_{—} | 8.7E−08 | 1.3E−07 | 16.7 | 0.0 | 2.3 | 0.1 | 7.3 | 0.6 | 38.97 |
| | | N325*I_F326*T_K329*H_{—} | | | | | | | | | |
| | | A330*H_F331*CT_I331*ET | | | | | | | | | |
| | 27769 | Template_12_14\|V266I_V273V_{—} | 8.0E−08 | 1.5E−07 | 18.1 | 0.0 | 2.1 | 0.1 | 8.5 | 0.7 | 33.96 |
| | | N325*I_F326*T_K329*H_{—} | | | | | | | | | |
| | | A330*A_F331*CT_I331*ET | | | | | | | | | |
| | 27770 | Template_12_14\|V266I_V273V_{—} | 7.3E−08 | 1.4E−07 | 19.7 | 0.0 | 2.1 | 0.1 | 9.2 | 0.8 | 42.44 |
| | | N325*I_F326*T_K329*K_{—} | | | | | | | | | |
| | | A330*D_F331*CT_I331*ET | | | | | | | | | |
| | 27771 | Template_12_14\|V266I_V273V_{—} | 6.6E−08 | 1.3E−07 | 22.0 | 0.1 | 2.4 | 0.1 | 9.1 | 0.8 | 36.13 |
| | | N325*I_F326*T_K329*K_{—} | | | | | | | | | |
| | | A330*E_F331*CT_I331*ET | | | | | | | | | |
| | 27772 | Template_12_14\|V266I_V273V_{—} | 1.1E−07 | 2.0E−07 | 13.7 | 0.0 | 1.5 | 0.0 | 9.0 | 0.8 | 38.60 |
| | | N325*I_F326*T_K329*K_{—} | | | | | | | | | |
| | | A330*N_F331*CT_I331*ET | | | | | | | | | |
| | 27773 | Template_12_14\|V266I_V273V_{—} | 1.6E−07 | 2.9E−07 | 9.0 | 0.0 | 1.1 | 0.0 | 8.3 | 0.7 | 36.29 |
| | | N325*I_F326*T_K329*K_{—} | | | | | | | | | |
| | | A330*S_F331*CT_I331*ET | | | | | | | | | |
| | 27774 | Template_12_14\|V266I_V273V_{—} | 1.2E−07 | 2.3E−07 | 11.9 | 0.0 | 1.4 | 0.0 | 8.7 | 0.7 | 34.07 |
| | | N325*I_F326*T_K329*K_{—} | | | | | | | | | |
| | | A330*H_F331*CT_I331*ET | | | | | | | | | |
| Strat4 | 27496 | Template_cl_4372-12_14-2\| | ND | ND | — | — | — | — | — | — | −91.42 |
| Template_12_14-2 | | F326*F_F331*CF_I331*EI | | | | | | | | | |
| | 27502 | Template_12_14-2\| | 1.3E−07 | 1.4E−07 | 11.2 | 0.0 | 2.2 | 0.1 | 5.1 | 0.4 | 10.80 |
| | | F326*T_F331*CT_I331*ET | | | | | | | | | |
| | 27495 | Template_12_14-2\| | 1.9E−07 | 4.2E−07 | 7.8 | 0.0 | 0.7 | 0.0 | 10.7 | 0.9 | 51.88 |
| | | F326*F_F331*CF_I331*EI | | | | | | | | | |
| | 27518 | Template_12_14-2\|V266I_F326*T_{—} | 1.2E−07 | 1.4E−07 | 12.3 | 0.0 | 2.2 | 0.1 | 5.6 | 0.5 | 14.24 |
| | | F331*CT_I331*ET | | | | | | | | | |
| | 27519 | Template_12_14-2\|V266L_F326*T_{—} | 1.4E−07 | 2.3E−07 | 10.2 | 0.0 | 1.4 | 0.0 | 7.5 | 0.6 | 26.59 |
| | | F331*CT_I331*ET | | | | | | | | | |
| | 27520 | Template_12_14-2\|V266F_F326*T_{—} | ND | ND | — | — | — | — | — | — | 10.81 |
| | | F331*CT_I331*ET | | | | | | | | | |
| | 27521 | Template_12_14-2\|V273I_F326*T_{—} | 9.9E−08 | 1.2E−07 | 14.6 | 0.0 | 2.6 | 0.1 | 5.5 | 0.5 | 8.23 |
| | | F331*CT_I331*ET | | | | | | | | | |
| | 27522 | Template_12_14-2\|V273L_F326*T_{—} | 4.2E−08 | 3.9E−08 | 34.5 | 0.1 | 7.9 | 0.2 | 4.4 | 0.4 | 4.2” |
| | | F331*CT_I331*ET | | | | | | | | | |
| | 27523 | Template_12_14-2\|V273F_F326*T_{—} | 1.8E−08 | 1.7E−08 | 78.8 | 0.2 | 17.9 | 0.5 | 4.4 | 0.4 | 4.50 |
| | | F331*CT_I331*ET | | | | | | | | | |
| | 27775 | Template_12_14-2\|V266V_V273V_{—} | 4.9E−08 | 6.3E−08 | 29.4 | 0.1 | 4.9 | 0.1 | 6.0 | 0.5 | 10.70 |
| | | D325*D_F326*T_K329*D_A330*D_{—} | | | | | | | | | |
| | | F331*CT_I331*ET | | | | | | | | | |
| | 27776 | Template_12_14-2\|V266V_V273V_{—} | ND | ND | — | — | — | — | — | — | 10.97 |
| | | D325*D_F326*T_K329*D_A330*E_{—} | | | | | | | | | |
| | | F331*CT_I331*ET | | | | | | | | | |
| | 27777 | Template_12_14-2\|V266V_V273V_{—} | 7.1E−08 | 7.7E−08 | 20.4 | 0.0 | 4.0 | 0.1 | 5.1 | 0.4 | 10.54 |
| | | D325*D_F326*T_K329*D_A330*N_{—} | | | | | | | | | |
| | | F331*CT_I331*ET | | | | | | | | | |
| | 27778 | Template_12_14-2\|V266V_V273V_{—} | 9.9E−08 | 1.2E−07 | 14.6 | 0.0 | 2.7 | 0.1 | 5.5 | 0.5 | 15.19 |
| | | D325*D_F326*T_K329*D_A330*S_{—} | | | | | | | | | |
| | | F331*CT_I331*ET | | | | | | | | | |
| | 27779 | Template_12_14-2\|V266V_V273V_{—} | ND | ND | — | — | — | — | — | — | |
| | | D325*D_F326*T_K329*D_A330*H_{—} | | | | | | | | | |
| | | F331*CT_I331*ET | | | | | | | | | |
| | 27780 | Template_12_14-2\|V266V_V273V_{—} | 6.9E−08 | 7.8E−08 | 21.0 | 0.1 | 3.9 | 0.1 | 5.3 | 0.5 | 14.87 |
| | | D325*D_F326*T_K329*D_A330*A_{—} | | | | | | | | | |
| | | F331*CT_I331*ET | | | | | | | | | |
| | 27781 | Template_12_14-2\|V266V_V273V_{—} | 5.3E−08 | 6.6E−08 | 27.0 | 0.1 | 4.7 | 0.1 | 5.7 | 0.5 | 13.95 |
| | | D325*D_F326*T_K329*E_A330*D_{—} | | | | | | | | | |
| | | F331*CT_I331*ET | | | | | | | | | |
| | 27782 | Template_12_14-2\|V266V_V273V_{—} | 5.5E−08 | 6.4E−08 | 26.2 | 0.1 | 4.8 | 0.1 | 5.5 | 0.5 | 12.26 |
| | | D325*D_F326*T_K329*E_A330*E_{—} | | | | | | | | | |
| | | F331*CT_I331*ET | | | | | | | | | |
| | 27783 | Template_12_14-2\|V266V_V273V_{—} | 7.4E−08 | 8.9E−08 | 19.5 | 0.0 | 3.5 | 0.1 | 5.6 | 0.5 | 10.97 |
| | | D325*D_F326*T_K329*E_A330*N_{—} | | | | | | | | | |
| | | F331*CT_I331*ET | | | | | | | | | |
| | 27784 | Template_12_14-2\|V266V_V273V_{—} | 9.0E−08 | 9.4E−08 | 16.1 | 0.0 | 3.3 | 0.1 | 4.9 | 0.4 | 15.47 |
| | | D325*D_F326*T_K329*E_A330*S_{—} | | | | | | | | | |
| | | F331*CT_I331*ET | | | | | | | | | |
| | 27785 | Template_12_14-2\|V266V_V273V_{—} | 8.0E−08 | 9.0E−08 | 18.1 | 0.0 | 3.4 | 0.1 | 5.3 | 0.4 | 11.71 |
| | | D325*D_F326*T_K329*E_A330*H_{—} | | | | | | | | | |
| | | F331*CT_I331*ET | | | | | | | | | |
| | 27786 | Template_12_14-2\|V266V_V273V_{—} | 7.2E−08 | 8.5E−08 | 20.1 | 0.0 | 3.6 | 0.1 | 5.5 | 0.5 | 12.07 |
| | | D325*D_F326*T_K329*E_A330*A_{—} | | | | | | | | | |
| | | F331*CT_I331*ET | | | | | | | | | |
| | 27787 | Template_12_14-2\|V266V_V273V_{—} | 7.6E−08 | 8.9E−08 | 19.0 | 0.0 | 3.5 | 0.1 | 5.5 | 0.5 | 11.89 |
| | | D325*D_F326*T_K329*N_A330*D_{—} | | | | | | | | | |
| | | F331*CT_I331*ET | | | | | | | | | |
| | 27788 | Template_12_14-2\|V266V_V273V_{—} | 6.3E−08 | 6.5E−08 | 22.9 | 0.1 | 4.8 | 0.1 | 4.8 | 0.4 | 11.02 |
| | | D325*D_F326*T_K329*N_A330*E_{—} | | | | | | | | | |
| | | F331*CT_I331*ET | | | | | | | | | |
| | 27789 | Template_12_14-2\|V266V_V273V_{—} | 1.1E−07 | 1.3E−07 | 13.3 | 0.0 | 2.5 | 0.1 | 5.4 | 0.5 | 10.95 |
| | | D325*D_F326*T_K329*N_A330*N_{—} | | | | | | | | | |
| | | F331*CT_I331*ET | | | | | | | | | |
| | 27790 | Template_12_14-2\|V266V_V273V_{—} | 1.5E−07 | 1.9E−07 | 9.5 | 0.0 | 1.6 | 0.0 | 5.8 | 0.5 | 16.00 |
| | | D325*D_F326*T_K329*N_A330*S_{—} | | | | | | | | | |
| | | F331*CT_I331*ET | | | | | | | | | |
| | 27791 | Template_12_14-2\|V266V_V273V_{—} | 1.1E−07 | 1.2E−07 | 13.1 | 0.0 | 2.5 | 0.1 | 5.2 | 0.4 | 10.79 |
| | | D325*D_F326*T_K329*N_A330*H_{—} | | | | | | | | | |
| | | F331*CT_I331*ET | | | | | | | | | |
| | 27792 | Template_12_14-2\|V266V_V273V_{—} | 9.9E−08 | 9.5E−08 | 14.6 | 0.0 | 3.2 | 0.1 | 4.5 | 0.4 | 10.74 |
| | | D325*D_F326*T_K329*N_A330*A_{—} | | | | | | | | | |
| | | F331*CT_I331*ET | | | | | | | | | |
| | 27793 | Template_12_14-2\|V266V_V273V_{—} | 7.9E−08 | 8.9E−08 | 18.2 | 0.0 | 3.5 | 0.1 | 5.2 | 0.4 | 12.00 |
| | | D325*D_F326*T_K329*S_A330*D_{—} | | | | | | | | | |
| | | F331*CT_I331*ET | | | | | | | | | |
| | 27794 | Template_12_14-2\|V266V_V273V_{—} | 6.7E−08 | 7.4E−08 | 21.6 | 0.1 | 4.2 | 0.1 | 5.2 | 0.4 | 11.85 |
| | | D325*D_F326*T_K329*S_A330*E_{—} | | | | | | | | | |
| | | F331*CT_I331*ET | | | | | | | | | |
| | 27795 | Template_12_14-2\|V266V_V273V_{—} | 1.2E−07 | 1.5E−07 | 12.0 | 0.0 | 2.0 | 0.1 | 5.9 | 0.5 | 13.66 |
| | | D325*D_F326*T_K329*S_A330*N_{—} | | | | | | | | | |
| | | F331*CT_I331*ET | | | | | | | | | |
| | 27796 | Template_12_14-2\|V266V_V273V_{—} | 1.3E−07 | 1.6E−07 | 10.9 | 0.0 | 1.9 | 0.1 | 5.6 | 0.5 | 14.20 |
| | | D325*D_F326*T_K329*S_A330*S_{——} | | | | | | | | | |
| | | F331*CT_I331*ET | | | | | | | | | |
| | 27797 | Template_12_14-2\|V266V_V273V_{—} | 1.4E−07 | 1.5E−07 | 10.3 | 0.0 | 2.1 | 0.1 | 5.0 | 0.4 | 12.56 |
| | | D325*D_F326*T_K329*S_A330*H_{—} | | | | | | | | | |
| | | F331*CT_I331*ET | | | | | | | | | |
| | 27798 | Template_12_14-2\|V266V_V273V_{—} | 1.1E−07 | 1.0E−07 | 13.2 | 0.0 | 3.1 | 0.1 | 4.3 | 0.4 | 3.96 |
| | | D325*D_F326*T_K329*S_A330*A_{—} | | | | | | | | | |
| | | F331*CT_I331*ET | | | | | | | | | |
| | 27799 | Template_12_14-2\|V266V_V273V_{—} | 7.7E−08 | 9.1E−08 | 18.8 | 0.0 | 3.4 | 0.1 | 5.6 | 0.5 | 10.55 |
| | | D325*D_F326*T_K329*H_A330*D_{—} | | | | | | | | | |
| | | F331*CT_I331*ET | | | | | | | | | |
| | 27800 | Template_12_14-2\|V266V_V273V_{—} | 7.2E−08 | 7.8E−08 | 20.0 | 0.0 | 3.9 | 0.1 | 5.1 | 0.4 | 9.41 |
| | | D325*D_F326*T_K329*H_A330*E_{—} | | | | | | | | | |
| | | F331*CT_I331*ET | | | | | | | | | |
| | 27801 | Template_12_14-2\|V266V_V273V_{—} | 1.5E−07 | 1.5E−07 | 9.9 | 0.0 | 2.0 | 0.1 | 4.9 | 0.4 | 11.49 |
| | | D325*D_F326*T_K329*H_A330*N_{—} | | | | | | | | | |
| | | F331*CT_I331*ET | | | | | | | | | |
| | 27802 | Template_12_14-2\|V266V_V273V_{—} | 1.5E−07 | 1.8E−07 | 9.8 | 0.0 | 1.7 | 0.0 | 5.7 | 0.5 | 13.95 |
| | | D325*D_F326*T_K329*H_A330*S_{—} | | | | | | | | | |
| | | F331*CT_I331*ET | | | | | | | | | |
| | 27803 | Template_12_14-2\|V266V_V273V_{—} | 1.2E−07 | 1.3E−07 | 12.3 | 0.0 | 2.4 | 0.1 | 5.1 | 0.4 | 10.88 |
| | | D325*D_F326*T_K329*H_A330*H_{—} | | | | | | | | | |
| | | F331*CT_I331*ET | | | | | | | | | |
| | 27804 | Template_12_14-2\|V266V_V273V_{—} | 9.6E−08 | 1.0E−07 | 15.1 | 0.0 | 3.0 | 0.1 | 5.1 | 0.4 | 9.67 |
| | | D325*D_F326*T_K329*H_A330*A_{—} | | | | | | | | | |
| | | F331*CT_I331*ET | | | | | | | | | |
| | 27805 | Template_12_14-2\|V266V_V273V_{—} | 9.8E−08 | 1.1E−07 | 14.7 | 0.0 | 2.9 | 0.1 | 5.2 | 0.4 | 11.17 |
| | | D325*D_F326*T_K329*K_A330*D_{—} | | | | | | | | | |
| | | F331*CT_I331*ET | | | | | | | | | |
| | 27806 | Template_12_14-2\|V266V_V273V_{—} | 8.5E−08 | 9.4E−08 | 16.9 | 0.0 | 3.3 | 0.1 | 5.2 | 0.4 | 10.04 |
| | | D325*D_F326*T_K329*K_A330*E_{—} | | | | | | | | | |
| | | F331*CT_I331*ET | | | | | | | | | |
| | 27807 | Template_12_14-2\|V266V_V273V_{—} | 1.7E−07 | 1.7E−07 | 8.4 | 0.0 | 1.8 | 0.1 | 4.7 | 0.4 | 10.03 |
| | | D325*D_F326*T_K329*K_A330*N_{—} | | | | | | | | | |
| | | F331*CT_I331*ET | | | | | | | | | |
| | 27808 | Template_12_14-2\|V266V_V273V_{—} | 1.9E−07 | 2.2E−07 | 7.5 | 0.0 | 1.4 | 0.0 | 5.3 | 0.5 | 13.13 |
| | | D325*D_F326*T_K329*K_A330*S_{—} | | | | | | | | | |
| | | F331*CT_I331*ET | | | | | | | | | |
| | 27809 | Template_12_14-2\|V266V_V273V_{—} | 2.0E−07 | 1.9E−07 | 7.4 | 0.0 | 1.6 | 0.0 | 4.7 | 0.4 | 10.19 |
| | | D325*D_F326*T_K329*K_A330*H_{—} | | | | | | | | | |
| | | F331*CT_I331*ET | | | | | | | | | |
| | 27810 | Template_12_14-2\|V266I_V273V_{—} | 4.7E−08 | 5.9E−08 | 30.7 | 0.1 | 5.2 | 0.1 | 5.9 | 0.5 | 4.80 |
| | | D325*D_F326*T_K329*D_A330*D_{—} | | | | | | | | | |
| | | F331*CT_I331*ET | | | | | | | | | |
| | 27811 | Template_12_14-2\|V266I_V273V_{—} | 4.0E−08 | 5.0E−08 | 35.9 | 0.1 | 6.1 | 0.2 | 5.9 | 0.5 | 8.18 |
| | | D325*D_F326*T_K329*D_A330*E_{—} | | | | | | | | | |
| | | F331*CT_I331*ET | | | | | | | | | |
| | 27812 | Template_12_14-2\|V266I_V273V_{—} | 5.9E−08 | 7.4E−08 | 24.4 | 0.1 | 4.2 | 0.1 | 5.9 | 0.5 | 11.81 |
| | | D325*D_F326*T_K329*D_A330*N_{—} | | | | | | | | | |
| | | F331*CT_I331*ET | | | | | | | | | |
| | 27813 | Template_12_14-2\|V266I_V273V_{—} | 8.7E−08 | 1.2E−07 | 16.6 | 0.0 | 2.6 | 0.1 | 6.4 | 0.5 | 16.83 |
| | | D325*D_F326*T_K329*D_A330*S_{—} | | | | | | | | | |
| | | F331*CT_I331*ET | | | | | | | | | |
| | 27814 | Template_12_14-2\|V266I_V273V_{—} | 6.5E−08 | 8.4E−08 | 22.2 | 0.1 | 3.7 | 0.1 | 6.0 | 0.5 | 12.36 |
| | | D325*D_F326*T_K329*D_A330*H_{—} | | | | | | | | | |
| | | F331*CT_I331*ET | | | | | | | | | |
| | 27815 | Template_12_14-2\|V266I_V273V_{—} | 6.3E−08 | 7.7E−08 | 22.9 | 0.1 | 4.0 | 0.1 | 5.7 | 0.5 | 13.32 |
| | | D325*D_F326*T_K329*D_A330*A_{—} | | | | | | | | | |
| | | F331*CT_I331*ET | | | | | | | | | |
| | 27816 | Template_12_14-2\|V266I_V273V_{—} | 5.7E−08 | 7.4E−08 | 25.6 | 0.1 | 4.2 | 0.1 | 6.1 | 0.5 | 15.54 |
| | | D325*D_F326*T_K329*E_A330*D_{—} | | | | | | | | | |
| | | F331*CT_I331*ET | | | | | | | | | |
| | 27817 | Template_12_14-2\|V266I_V273V_{—} | 5.7E−08 | 6.7E−08 | 25.4 | 0.1 | 4.6 | 0.1 | 5.6 | 0.5 | 14.08 |
| | | D325*D_F326*T_K329*E_A330*E_{—} | | | | | | | | | |
| | | F331*CT_I331*ET | | | | | | | | | |
| | 27818 | Template_12_14-2\|V266I_V273V_{—} | ND | ND | — | — | — | — | — | — | 5.70 |
| | | D325*D_F326*T_K329*E_A330*N_{—} | | | | | | | | | |
| | | F331*CT_I331*ET | | | | | | | | | |
| | 27819 | Template_12_14-2\|V266I_V273V_{—} | 8.5E−08 | 1.2E−07 | 17.1 | 0.0 | 2.7 | 0.1 | 6.4 | 0.5 | 17.30 |
| | | D325*D_F326*T_K329*E_A330*S_{—} | | | | | | | | | |
| | | F331*CT_I331*ET | | | | | | | | | |
| | 27820 | Template_12_14-2\|V266I_V273V_{—} | 7.0E−08 | 8.9E−08 | 20.6 | 0.0 | 3.5 | 0.1 | 6.0 | 0.5 | 15.71 |
| | | D325*D_F326*T_K329*E_A330*H_{—} | | | | | | | | | |
| | | F331*CT_I331*ET | | | | | | | | | |
| | 27821 | Template_12_14-2\|V266I_V273V_{—} | 6.1E−08 | 7.9E−08 | 23.6 | 0.1 | 3.9 | 0.1 | 6.0 | 0.5 | 15.02 |
| | | [D325*D_F326*T_K329*E_A330*A_{—} | | | | | | | | | |
| | | F331*CT_I331*ET | | | | | | | | | |
| | 27822 | Template_12_14-2\|V266I_V273V_{—} | 5.8E−08 | 7.3E−08 | 24.9 | 0.1 | 4.2 | 0.1 | 5.9 | 0.5 | 12.00 |
| | | D325*D_F326*T_K329*N_A330*D_{—} | | | | | | | | | |
| | | F331*CT_I331*ET | | | | | | | | | |
| | 27823 | Template_12_14-2\|V266I_V273V_{—} | 5.7E−08 | 6.4E−08 | 25.3 | 0.1 | 4.8 | 0.1 | 5.3 | 0.4 | 7.16 |
| | | D325*D_F326*T_K329*N_A330*E_{—} | | | | | | | | | |
| | | F331*CT_I331*ET | | | | | | | | | |
| | 27824 | Template_12_14-2\|V266I_V273V_{—} | ND | ND | — | — | — | — | — | — | 10.38 |
| | | D325*D_F326*T_K329*N_A330*N_{—} | | | | | | | | | |
| | | F331*CT_I331*ET | | | | | | | | | |
| | 27825 | Template_12_14-2\|V266I_V273V_{—} | 1.6E−07 | 2.1E−07 | 8.9 | 0.0 | 1.5 | 0.0 | 6.0 | 0.5 | 19.82 |
| | | D325*D_F326*T_K329*N_A330*S_{—} | | | | | | | | | |
| | | F331*CT_I331*ET | | | | | | | | | |
| | 27826 | Template_12_14-2\|V266I_V273V_{—} | 1.0E−07 | 1.3E−07 | 13.9 | 0.0 | 2.4 | 0.1 | 5.9 | 0.5 | 5.53 |
| | | D325*D_F326*T_K329*N_A330*H_{—} | | | | | | | | | |
| | | F331*CT_I331*ET | | | | | | | | | |
| | 27827 | Template_12_14-2\|V266I_V273V_{—} | 9.3E−08 | 1.1E−07 | 15.5 | 0.0 | 2.8 | 0.1 | 5.5 | 0.5 | 1.76 |
| | | D325*D_F326*T_K329*N_A330*A_{—} | | | | | | | | | |
| | | F331*CT_I331*ET | | | | | | | | | |
| | 27828 | Template_12_14-2\|V266I_V273V_{—} | 6.0E−08 | 7.2E−08 | 24.2 | 0.1 | 4.3 | 0.1 | 5.7 | 0.5 | 14.48 |
| | | D325*D_F326*T_K329*S_A330*D_{—} | | | | | | | | | |
| | | F331*CT_I331*ET | | | | | | | | | |
| | 27829 | Template_12_14-2\|V266I_V273V_{—} | 5.8E−08 | 7.2E−08 | 25.1 | 0.1 | 4.3 | 0.1 | 5.9 | 0.5 | 13.64 |
| | | D325*D_F326*T_K329*S_A330*E_{—} | | | | | | | | | |
| | | F331*CT_I331*ET | | | | | | | | | |
| | 27830 | Template_12_14-2\|V266I_V273V_{—} | ND | ND | — | — | — | — | — | — | 9.26 |
| | | D325*D_F326*T_K329*S_A330*N_{—} | | | | | | | | | |
| | | F331*CT_I331*ET | | | | | | | | | |
| | 27831 | Template_12_14-2\|V266I_V273V_{—} | 1.4E−07 | 1.9E−07 | 10.5 | 0.0 | 1.6 | 0.0 | 6.5 | 0.5 | 17.99 |
| | | D325*D_F326*T_K329*S_A330*S_{—} | | | | | | | | | |
| | | F331*CT_I331*ET | | | | | | | | | |
| | 27832 | Template_12_14-2\|V266I_V273V_{—} | 1.4E−07 | 1.7E−07 | 10.0 | 0.0 | 1.8 | 0.1 | 5.6 | 0.5 | 15.47 |
| | | D325*D_F326*T_K329*S_A330*H_{—} | | | | | | | | | |
| | | F331*CT_I331*ET | | | | | | | | | |
| | 27833 | Template_12_14-2\|V266I_V273V_{—} | 1.1E−07 | 1.3E−07 | 13.6 | 0.0 | 2.4 | 0.1 | 5.7 | 0.5 | 15.53 |
| | | D325*D_F326*T_K329*S_A330*A_{—} | | | | | | | | | |
| | | F331*CT_I331*ET | | | | | | | | | |
| | 27834 | Template_12_14-2\|V266I_V273V_{—} | 6.6E−08 | 7.0E−08 | 22.0 | 0.1 | 4.4 | 0.1 | 5.0 | 0.4 | 12.31 |
| | | D325*D_F326*T_K329*H_A330*D_{—} | | | | | | | | | |
| | | F331*CT_I331*ET | | | | | | | | | |
| | 27835 | Template_12_14-2\|V266I_V273V_{—} | 4.9E−08 | 5.5E−08 | 29.3 | 0.1 | 5.6 | 0.2 | 5.3 | 0.4 | 11.18 |
| | | D325*D_F326*T_K329*H_A330*E_{—} | | | | | | | | | |
| | | F331*CT_I331*ET | | | | | | | | | |
| | 27836 | Template_12_14-2\|V266I_V273V_{—} | 1.2E−07 | 1.5E−07 | 11.9 | 0.0 | 2.1 | 0.1 | 5.7 | 0.5 | 14.06 |
| | | D325*D_F326*T_K329*H_A330*N_{—} | | | | | | | | | |
| | | F331*CT_I331*ET | | | | | | | | | |
| | 27837 | Template_12_14-2\|V266I_V273V_{—} | 1.0E−07 | 1.2E−07 | 14.1 | 0.0 | 2.5 | 0.1 | 5.7 | 0.5 | |
| | | D325*D_F326*T_K329*H_A330*S_{—} | | | | | | | | | |
| | | F331*CT_I331*ET | | | | | | | | | |
| | 27838 | Template_12_14-2\|V266I_V273V_{—} | 1.2E−07 | 1.2E−07 | 11.9 | 0.0 | 2.5 | 0.1 | 4.7 | 0.4 | 12.66 |
| | | D325*D_F326*T_K329*H_A330*H_{—} | | | | | | | | | |
| | | F331*CT_I331*ET | | | | | | | | | |
| | 27839 | Template_12_14-2\|V266I_V273V_{—} | 9.7E−08 | 1.0E−07 | 14.9 | 0.0 | 3.1 | 0.1 | 4.9 | 0.4 | 10.79 |
| | | D325*D_F326*T_K329*H_A330*A_{—} | | | | | | | | | |
| | | F331*CT_I331*ET | | | | | | | | | |
| | 27840 | Template_12_14-2\|V266I_V273V_{—} | 8.7E−08 | 1.1E−07 | 16.6 | 0.0 | 2.9 | 0.1 | 5.8 | 0.5 | 14.18 |
| | | D325*D_F326*T_K329*K_A330*D_{—} | | | | | | | | | |
| | | F331*CT_I331*ET | | | | | | | | | |
| | 27841 | Template_12_14-2\|V266I_V273V_{—} | 8.1E−08 | 9.5E−08 | 17.8 | 0.0 | 3.3 | 0.1 | 5.5 | 0.5 | 12.69 |
| | | D325*D_F326*T_K329*K_A330*E_{—} | | | | | | | | | |
| | | F331*CT_I331*ET | | | | | | | | | |
| | 27842 | Template_12_14-2\|V266I_V273V_{—} | 1.5E−07 | 1.7E−07 | 9.6 | 0.0 | 1.9 | 0.1 | 5.2 | 0.4 | 13.68 |
| | | D325*D_F326*T_K329*K_A330*N_{—} | | | | | | | | | |
| | | F331*CT_I331*ET | | | | | | | | | |
| | 27843 | Template_12_14-2\|V266I_V273V_{—} | 1.7E−07 | 2.0E−07 | 8.6 | 0.0 | 1.5 | 0.0 | 5.6 | 0.5 | −3.11 |
| | | D325*D_F326*T_K329*K_A330*S_{—} | | | | | | | | | |
| | | 1_F331*CT_I331*ET | | | | | | | | | |
| | 27844 | Template_12_14-2\|V266I_V273V_{—} | 1.5E−07 | 1.7E−07 | 9.6 | 0.0 | 1.8 | 0.1 | 5.3 | 0.5 | 12.33 |
| | | D325*D_F326*T_K329*K_A330*H_{—} | | | | | | | | | |
| | | F331*CT_I331*ET | | | | | | | | | |
| Strat4 | 27497 | Template_14_0 | 1.2E−07 | 1.2E−07 | 12.3 | 0.0 | 2.6 | 0.1 | 4.8 | 0.4 | 9.13 |
| Template_14_0 | 27498 | Template_cl_4372- | ND | ND | — | — | — | — | — | — | −58.18 |
| | | 14_0\|V326*V_Y331*CY_L331*EL_{—} | | | | | | | | | |
| | | L331*GL | | | | | | | | | |
| | 27503 | Template_14_0\|V326*T_Y331*CT_{—} | 9.0E−08 | 8.9E−08 | 16.0 | 0.0 | 3.5 | 0.1 | 4.6 | 0.4 | 8.30 |
| | | L331*ET_L331*GT | | | | | | | | | |
| | 27524 | Template_14_0\|V266I_V326*T_{—} | 8.5E−08 | 9.5E−08 | 17.1 | 0.0 | 3.2 | 0.1 | 5.3 | 0.4 | 10.87 |
| | | Y331*CT_L331*ET_L331*GT | | | | | | | | | |
| | 27525 | Template_14_0\|V266L_V326*T_{—} | 1.3E−07 | 1.8E−07 | 11.0 | 0.0 | 1.7 | 0.0 | 6.5 | 0.6 | 20.48 |
| | | Y331*CT_L331*ET_L331*GT | | | | | | | | | |
| | 27526 | Template_14_0\|V266F_V326*T_{—} | 5.5E−07 | 5.0E−07 | 2.6 | 0.0 | 0.6 | 0.0 | 4.3 | 0.4 | 15.79 |
| | | Y331*CT_L331*ET_L331*GT | | | | | | | | | |
| | 27527 | Template_14_0\|V273I_V326*T_{—} | 7.3E−08 | 7.3E−08 | 19.7 | 0.0 | 4.2 | 0.1 | 4.7 | 0.4 | 8.15 |
| | | Y331*CT_L331*ET_L331*GT | | | | | | | | | |
| | 27528 | Template_14_0\|V273L_V326*T_{—} | 5.7E−08 | 5.4E−08 | 25.2 | 0.1 | 5.8 | 0.2 | 4.4 | 0.4 | 6.22 |
| | | Y331*CT_L331*ET_L331*GT | | | | | | | | | |
| | 27529 | Template_14_0\|V273F_V326*T_{—} | 3.2E−08 | 3.3E−08 | 45.3 | 0.1 | 9.4 | 0.3 | 4.8 | 0.4 | 7.26 |
| | | Y331*CT_L331*ET_L331*GT | | | | | | | | | |
| | 27530 | Template_14_0\|Q325*V_V326*T_{—} | 7.9E−08 | 1.0E−07 | 18.3 | 0.0 | 3.0 | 0.1 | 6.0 | 0.5 | 7.57 |
| | | Y331*CT_L331*ET_L331*GT | | | | | | | | | |
| | 27531 | Template_14_0/Q325*I_V326*T_{—} | 3.3E−08 | 3.6E−08 | 43.2 | 0.1 | 8.5 | 0.2 | 5.1 | 0.4 | 5.48 |
| | | Y331*CT_L331*ET_L331*GT | | | | | | | | | |
| | 27532 | Template_14_0\|Q325*L_V326*T_{—} | 1.7E−08 | 1.9E−08 | 86.6 | 0.2 | 16.1 | 0.5 | 5.4 | 0.5 | 2.25 |
| | | Y331*CT_L331*ET_L331*GT | | | | | | | | | |
| | 27533 | Template_14_0\|Q325*F_V326*T_{—} | 2.1E−08 | 2.3E−08 | 69.7 | 0.2 | 13.3 | 0.4 | 5.2 | 0.4 | 10.36 |
| | | Y331*CT_L331*ET_L331*GT | | | | | | | | | |
| | 27845 | Template_14_0\|V326*T_D329*D_{—} | 6.6E−08 | 7.1E−08 | 22.0 | 0.1 | 4.4 | 0.1 | 5.1 | 0.4 | 2.98 |
| | | A330*D_T331*D_Y331*CT_{—} | | | | | | | | | |
| | | L331*ET_L331*GT | | | | | | | | | |
| | 27846 | Template_14_0\|V326*T_D329*D_{—} | 7.0E−08 | 7.1E−08 | 20.6 | 0.0 | 4.4 | 0.1 | 4.7 | 0.4 | 8.51 |
| | | A330*D_T331*E_Y331*CT_{—} | | | | | | | | | |
| | | L331*ET_L331*GT | | | | | | | | | |
| | 27847 | Template_14_0\|V326*T_D329*D_{—} | 8.1E−08 | 7.8E−08 | 17.8 | 0.0 | 3.9 | 0.1 | 4.5 | 0.4 | 7.30 |
| | | A330*D_T331*N_Y331*CT_{—} | | | | | | | | | |
| | | L331*ET_L331*GT | | | | | | | | | |
| | 27848 | Template_14_0\|V326*T_D329*D_{—} | 8.6E−08 | 8.7E−08 | 16.8 | 0.0 | 3.6 | 0.1 | 4.7 | 0.4 | 8.76 |
| | | A330*D_T331*S_Y331*CT_{—} | | | | | | | | | |
| | | L331*ET_L331*GT | | | | | | | | | |
| | 27849 | Template_14_0\|V326*T_D329*D_{—} | 9.2E−08 | 8.9E−08 | 15.7 | 0.0 | 3.5 | 0.1 | 4.5 | 0.4 | 8.47 |
| | | A330*D_T331*T_Y331*CT_{—} | | | | | | | | | |
| | | L331*ET_L331*GT | | | | | | | | | |
| | 27850 | Template_14_0\|V326*T_D329*D_{—} | 7.0E−08 | 7.4E−08 | 20.7 | 0.0 | 4.1 | 0.1 | 5.0 | 0.4 | 8.16 |
| | | A330*E_T331*D_Y331*CT_{—} | | | | | | | | | |
| | | L331*ET_L331*GT | | | | | | | | | |
| | 27851 | Template_14_0\|V326*T_D329*D_{—} | 6.2E−08 | 6.7E−08 | 23.1 | 0.1 | 4.6 | 0.1 | 5.0 | 0.4 | 8.49 |
| | | A330*E_T331*E_Y331*CT_{—} | | | | | | | | | |
| | | L331*ET_L331*GT | | | | | | | | | |
| | 27852 | Template_14_0\|V326*T_D329*D_{—} | 7.8E−08 | 8.1E−08 | 18.4 | 0.0 | 3.8 | 0.1 | 4.8 | 0.4 | 8.25 |
| | | A330*E_T331*N_Y331*CT_{—} | | | | | | | | | |
| | | L331*ET_L331*GT | | | | | | | | | |
| | 27853 | Template_14_0\|V326*T_D329*D_{—} | 7.5E−08 | 7.4E−08 | 19.4 | 0.0 | 4.2 | 0.1 | 4.6 | 0.4 | −3.24 |
| | | A330*E_T331*S_Y331*CT_{—} | | | | | | | | | |
| | | L331*ET_L331*GT | | | | | | | | | |
| | 27854 | Template_14_0\|V326*T_D329*D_{—} | 6.5E−08 | 7.3E−08 | 22.1 | 0.1 | 4.2 | 0.1 | 5.2 | 0.4 | |
| | | A330*E_T331*T_Y331*CT_{—} | | | | | | | | | |
| | | L331*ET_L331*GT | | | | | | | | | |
| | 27855 | Template_14_0\|V326*T_D329*D_{—} | 7.1E−08 | 7.2E−08 | 20.4 | 0.0 | 4.3 | 0.1 | 4.8 | 0.4 | 7.44 |
| | | A330*N_T331*D_Y331*CT_{—} | | | | | | | | | |
| | | L331*ET_L331*GT | | | | | | | | | |
| | 27856 | Template_14_0\|V326*T_D329*D_{—} | 7.8E−08 | 8.3E−08 | 18.5 | 0.0 | 3.7 | 0.1 | 5.0 | 0.4 | 7.46 |
| | | A330*N_T331*E_Y331*CT_{—} | | | | | | | | | |
| | | L331*ET_L331*GT | | | | | | | | | |
| | 27857 | Template_14_0\|V326*T_D329*D_{—} | 8.5E−08 | 8.9E−08 | 16.9 | 0.0 | 3.5 | 0.1 | 4.9 | 0.4 | 7.81 |
| | | A330*N_T331*N_Y331*CT_{—} | | | | | | | | | |
| | | L331*ET_L331*GT | | | | | | | | | |
| | 27858 | Template_14_0\|V326*T_D329*D_{—} | 8.7E−08 | 8.9E−08 | 16.6 | 0.0 | 3.5 | 0.1 | 4.8 | 0.4 | 7.47 |
| | | A330*N_T331*S_Y331*CT_{—} | | | | | | | | | |
| | | L331*ET_L331*GT | | | | | | | | | |
| | 27859 | Template_14_0\|V326*T_D329*D_{—} | 7.7E−08 | 7.9E−08 | 18.7 | 0.0 | 3.9 | 0.1 | 4.8 | 0.4 | 7.95 |
| | | A330*N_T331*T_Y331*CT_{—} | | | | | | | | | |
| | | L331*ET_L331*GT | | | | | | | | | |
| | 27860 | Template_14_0\|V326*T_D329*D_{—} | 6.4E−08 | 5.7E−08 | 22.5 | 0.1 | 5.4 | 0.2 | 4.2 | 0.4 | 11.13 |
| | | A330*S_T331*D_Y331*CT_{—} | | | | | | | | | |
| | | L331*ET_L331*GT | | | | | | | | | |
| | 27861 | Template_14_0\|V326*T_D329*D_{—} | 7.4E−08 | 7.5E−08 | 19.4 | 0.0 | 4.1 | 0.1 | 4.7 | 0.4 | 8.16 |
| | | A330*S_T331*E_Y331*CT_{—} | | | | | | | | | |
| | | L331*ET_L331*GT | | | | | | | | | |
| | 27862 | Template_14_0\|V326*T_D329*D_{—} | 9.7E−08 | 9.3E−08 | 14.9 | 0.0 | 3.3 | 0.1 | 4.5 | 0.4 | 7.45 |
| | | A330*S_T331*N_Y331*CT_{—} | | | | | | | | | |
| | | L331*ET_L331*GT | | | | | | | | | |
| | 27863 | Template_14_0\|V326*T_D329*D_{—} | 9.7E−08 | 9.7E−08 | 14.8 | 0.0 | 3.2 | 0.1 | 4.7 | 0.4 | 8.86 |
| | | A330*S_T331*S_Y331*CT_{—} | | | | | | | | | |
| | | L331*ET_L331*GT | | | | | | | | | |
| | 27864 | Template_14_0\|V326*T_D329*D_{—} | 9.4E−08 | 9.1E−08 | 15.4 | 0.0 | 3.4 | 0.1 | 4.6 | 0.4 | 7.94 |
| | | A330*S_T331*T_Y331*CT_{—} | | | | | | | | | |
| | | L331*ET_L331*GT | | | | | | | | | |
| | 27865 | Template_14_0\|V326*T_D329*D_{—} | 8.4E−08 | 8.6E−08 | 17.1 | 0.0 | 3.6 | 0.1 | 4.8 | 0.4 | 8.57 |
| | | A330*A_T331*D_Y331*CT_{—} | | | | | | | | | |
| | | L331*ET_L331*GT | | | | | | | | | |
| | 27866 | Template_14_0\|V326*T_D329*D_{—} | 6.5E−08 | 7.8E−08 | 22.3 | 0.1 | 4.0 | 0.1 | 5.6 | 0.5 | 8.05 |
| | | A330*A_T331*E_Y331*CT_{—} | | | | | | | | | |
| | | L331*ET_L331*GT | | | | | | | | | |
| | 27867 | Template_14_0\|V326*T_D329*D_{—} | 8.1E−08 | 8.0E−08 | 17.9 | 0.0 | 3.8 | 0.1 | 4.7 | 0.4 | 7.90 |
| | | A330*A_T331*N_Y331*CT_{—} | | | | | | | | | |
| | | L331*ET_L331*GT | | | | | | | | | |
| | 27868 | Template_14_0\|V326*T_D329*D_{—} | 8.7E−08 | 9.0E−08 | 16.6 | 0.0 | 3.4 | 0.1 | 4.8 | 0.4 | 7.48 |
| | | A330*A_T331*S_Y331*CT_{—} | | | | | | | | | |
| | | L331*ET_L331*GT | | | | | | | | | |
| | 27869 | Template_14_0\|V326*T_D329*E_{—} | 6.2E−08 | 6.5E−08 | 23.2 | 0.1 | 4.8 | 0.1 | 4.9 | 0.4 | 7.74 |
| | | A330*D_T331*D_Y331*CT_{—} | | | | | | | | | |
| | | L331*ET_L331*GT | | | | | | | | | |
| | 27870 | Template_14_0\|V326*T_D329*E_{—} | 6.2E−08 | 6.4E−08 | 23.3 | 0.1 | 4.8 | 0.1 | 4.9 | 0.4 | 7.93 |
| | | A330*D_T331*E_Y331*CT_{—} | | | | | | | | | |
| | | L331*ET_L331*GT | | | | | | | | | |
| | 27871 | Template_14_0\|V326*T_D329*E_{—} | 7.5E−08 | 7.5E−08 | 19.3 | 0.0 | 4.1 | 0.1 | 4.7 | 0.4 | −1.01 |
| | | A330*D_T331*N_Y331*CT_{—} | | | | | | | | | |
| | | L331*ET_L331*GT | | | | | | | | | |
| | 27872 | Template_14_0\|V326*T_D329*E_{—} | 7.6E−08 | 7.9E−08 | 19.1 | 0.0 | 3.9 | 0.1 | 4.9 | 0.4 | 6.92 |
| | | A330*D_T331*S_Y331*CT_{—} | | | | | | | | | |
| | | L331*ET_L331*GT | | | | | | | | | |
| | 27873 | Template_14_0\|V326*T_D329*E_{—} | ND | ND | — | — | — | — | — | — | |
| | | A330*D_T331*T_Y331*CT_{—} | | | | | | | | | |
| | | L331*ET_L331*GT | | | | | | | | | |
| | 27874 | Template_14_0\|V326*T_D329*E_{—} | 6.6E−08 | 6.8E−08 | 22.0 | 0.1 | 4.5 | 0.1 | 4.9 | 0.4 | 7.87 |
| | | A330*E_T331*D_Y331*CT_{—} | | | | | | | | | |
| | | L331*ET_L331*GT | | | | | | | | | |
| | 27875 | Template_14_0\|V326*T_D329*E_{—} | 6.0E−08 | 6.3E−08 | 24.0 | 0.1 | 4.9 | 0.1 | 4.9 | 0.4 | 8.08 |
| | | A330*E_T331*E_Y331*CT_{—} | | | | | | | | | |
| | | L331*ET_L331*GT | | | | | | | | | |
| | 27876 | Template_14_0\|V326*T_D329*E_{—} | 7.1E−08 | 7.4E−08 | 20.4 | 0.0 | 4.2 | 0.1 | 4.9 | 0.4 | 7.81 |
| | | A330*E_T331*N_Y331*CT_{—} | | | | | | | | | |
| | | L331*ET_L331*GT | | | | | | | | | |
| | 27877 | Template_14_0\|V326*T_D329*E_{—} | ND | ND | — | — | — | — | — | — | |
| | | A330*E_T331*S_Y331*CT_{—} | | | | | | | | | |
| | | L331*ET_L331*GT | | | | | | | | | |
| | 27878 | Template_14_0\|V326*T_D329*E_{—} | 7.3E−08 | 6.9E−08 | 19.7 | 0.0 | 4.5 | 0.1 | 4.4 | 0.4 | 8.59 |
| | | A330*E_T331*T_Y331*CT_{—} | | | | | | | | | |
| | | L331*ET_L331*GT | | | | | | | | | |
| | 27879 | Template_14_0\|V326*T_D329*E_{—} | 7.6E−08 | 7.4E−08 | 19.1 | 0.0 | 4.2 | 0.1 | 4.6 | 0.4 | 7.74 |
| | | A330*N_T331*D_Y331*CT_{—} | | | | | | | | | |
| | | L331*ET_L331*GT | | | | | | | | | |
| | 27880 | Template_14_0\|V326*T_D329*E_{—} | 7.3E−08 | 7.6E−08 | 19.7 | 0.0 | 4.1 | 0.1 | 4.8 | 0.4 | 7.80 |
| | | A330*N_T331*E_Y331*CT_{—} | | | | | | | | | |
| | | L331*ET_L331*GT | | | | | | | | | |
| | 27881 | Template_14_0\|V326*T_D329*E_{—} | 8.1E−08 | 8.2E−08 | 17.9 | 0.0 | 3.8 | 0.1 | 4.7 | 0.4 | 8.16 |
| | | A330*N_T331*N_Y331*CT_{—} | | | | | | | | | |
| | | L331*ET_L331*GT | | | | | | | | | |
| | 27882 | Template_14_0\|V326*T_D329*E_{—} | 7.2E−08 | 7.3E−08 | 20.1 | 0.0 | 4.2 | 0.1 | 4.8 | 0.4 | 8.10 |
| | | A330*N_T331*S_Y331*CT_{—} | | | | | | | | | |
| | | L331*ET_L331*GT | | | | | | | | | |
| | 27883 | Template_14_0\|V326*T_D329*E_{—} | 6.7E−08 | 7.0E−08 | 21.5 | 0.1 | 4.4 | 0.1 | 4.9 | 0.4 | 7.83 |
| | | A330*N_T331*T_Y331*CT_{—} | | | | | | | | | |
| | | L331*ET_L331*GT | | | | | | | | | |
| | 27884 | Template_14_0\|V326*T_D329*E_{—} | 6.7E−08 | 7.0E−08 | 21.4 | 0.1 | 4.4 | 0.1 | 4.9 | 0.4 | 8.15 |
| | | A330*S_T331*D_Y331*CT_{—} | | | | | | | | | |
| | | L331*ET_L331*GT | | | | | | | | | |
| | 27885 | Template_14_0\|V326*T_D329*E_{—} | 6.5E−08 | 7.0E−08 | 22.3 | 0.1 | 4.4 | 0.1 | 5.1 | 0.4 | 10.98 |
| | | A330*S_T331*E_Y331*CT_{—} | | | | | | | | | |
| | | L331*ET_L331*GT | | | | | | | | | |
| | 27886 | Template_14_0\|V326*T_D329*E_{—} | 8.2E−08 | 7.9E−08 | 17.6 | 0.0 | 3.9 | 0.1 | 4.5 | 0.4 | 7.05 |
| | | A330*S_T331*N_Y331*CT_{—} | | | | | | | | | |
| | | L331*ET_L331*GT | | | | | | | | | |
| | 27887 | Template_14_0\|V326*T_D329*E_{—} | 8.2E−08 | 8.4E−08 | 17.7 | 0.0 | 3.7 | 0.1 | 4.8 | 0.4 | 7.97 |
| | | A330*S_T331*S_Y331*CT_{—} | | | | | | | | | |
| | | L331*ET_L331*GT | | | | | | | | | |
| | 27888 | Template_14_0\|V326*T_D329*E_{—} | 7.4E−08 | 7.6E−08 | 19.7 | 0.0 | 4.0 | 0.1 | 4.9 | 0.4 | 7.75 |
| | | A330*S_T331*T_Y331*CT_{—} | | | | | | | | | |
| | | L331*ET_L331*GT | | | | | | | | | |
| | 27889 | Template_14_0\|V326*T_D329*E_{—} | 6.1E−08 | 6.9E−08 | 23.6 | 0.1 | 4.5 | 0.1 | 5.3 | 0.4 | 8.41 |
| | | A330*A_T331*D_Y331*CT_{—} | | | | | | | | | |
| | | L331*ET_L331*GT | | | | | | | | | |
| | 27890 | Template_14_0\|V326*T_D329*E_{—} | ND | ND | — | — | — | — | — | — | |
| | | A330*A_T331*E_Y331*CT_{—} | | | | | | | | | |
| | | L331*ET_L331*GT | | | | | | | | | |
| | 27891 | Template_14_0\|V326*T_D329*E_{—} | 7.9E−08 | 7.3E−08 | 18.4 | 0.0 | 4.2 | 0.1 | 4.4 | 0.4 | 7.57 |
| | | A330*A_T331*N_Y331*CT_{—} | | | | | | | | | |
| | | L331*ET_L331*GT | | | | | | | | | |
| | 27892 | Template_14_0\|V326*T_D329*E_{—} | 7.8E−08 | 7.8E−08 | 18.5 | 0.0 | 4.0 | 0.1 | 4.6 | 0.4 | 7.24 |
| | | A330*A_T331*S_Y331*CT_{—} | | | | | | | | | |
| | | L331*ET_L331*GT | | | | | | | | | |
| | 27893 | Template_14_0\|V326*T_D329*E_{—} | 7.9E−08 | 8.1E−08 | 18.2 | 0.0 | 3.8 | 0.1 | 4.8 | 0.4 | 7.39 |
| | | A330*A_T331*T_Y331*CT_{—} | | | | | | | | | |
| | | L331*ET_L331*GT | | | | | | | | | |
| | 27894 | Template_14_0\|V326*T_D329*N_{—} | 7.4E−08 | 7.4E−08 | 19.6 | 0.0 | 4.2 | 0.1 | 4.7 | 0.4 | 7.45 |
| | | A330*D_T331*D_Y331*CT_{—} | | | | | | | | | |
| | | L331*ET_L331*GT | | | | | | | | | |
| | 27895 | Template_14_0\|V326*T_D329*N_{—} | 8.2E−08 | 7.8E−08 | 17.7 | 0.0 | 3.9 | 0.1 | 4.5 | 0.4 | 7.57 |
| | | A330*D_T331*E_Y331*CT_{—} | | | | | | | | | |
| | | L331*ET_L331*GT | | | | | | | | | |
| | 27896 | Template_14_0\|V326*T_D329*N_{—} | 7.5E−08 | 7.9E−08 | 19.3 | 0.0 | 3.9 | 0.1 | 4.9 | 0.4 | 7.20 |
| | | A330*D_T331*N_Y331*CT_{—} | | | | | | | | | |
| | | L331*ET_L331*GT | | | | | | | | | |
| | 27897 | Template_14_0\|V326*T_D329*N_{—} | 7.6E−08 | 7.6E−08 | 19.1 | 0.0 | 4.1 | 0.1 | 4.7 | 0.4 | 8.88 |
| | | A330*E_T331*D_Y331*CT_{—} | | | | | | | | | |
| | | L331*ET_L331*GT | | | | | | | | | |
| | 27898 | Template_14_0\|V326*T_D329*N_{—} | 7.3E−08 | 7.6E−08 | 19.8 | 0.0 | 4.0 | 0.1 | 4.9 | 0.4 | 8.22 |
| | | A330*E_T331*E_Y331*CT_{—} | | | | | | | | | |
| | | L331*ET_L331*GT | | | | | | | | | |
| | 27899 | Template_14_0\|V326*T_D329*N_{—} | 8.4E−08 | 8.8E−08 | 17.2 | 0.0 | 3.5 | 0.1 | 4.9 | 0.4 | 8.98 |
| | | A330*E_T331*N_Y331*CT_{—} | | | | | | | | | |
| | | L331*ET_L331*GT | | | | | | | | | |
| | 27900 | Template_14_0\|V326*T_D329*N_{—} | 7.8E−08 | 7.5E−08 | 18.6 | 0.0 | 4.1 | 0.1 | 4.5 | 0.4 | 8.33 |
| | | A330*N_T331*D_Y331*CT_{—} | | | | | | | | | |
| | | L331*ET_L331*GT | | | | | | | | | |
| | 27901 | Template_14_0\|V326*T_D329*N_{—} | 8.0E−08 | 8.2E−08 | 18.0 | 0.0 | 3.8 | 0.1 | 4.8 | 0.4 | 8.14 |
| | | A330*N_T331*E_Y331*CT_{—} | | | | | | | | | |
| | | L331*ET_L331*GT | | | | | | | | | |
| | 27902 | Template_14_0\|V326*T_D329*N_{—} | 9.4E−08 | 9.5E−08 | 15.4 | 0.0 | 3.2 | 0.1 | 4.8 | 0.4 | 7.11 |
| | | A330*N_T331*N_Y331*CT_{—} | | | | | | | | | |
| | | L331*ET_L331*GT | | | | | | | | | |
| | 27903 | Template_14_0\|V326*T_D329*N_{—} | 8.5E−08 | 8.8E−08 | 17.0 | 0.0 | 3.5 | 0.1 | 4.8 | 0.4 | 7.48 |
| | | A330*S_T331*D_Y331*CT_{—} | | | | | | | | | |
| | | L331*ET_L331*GT | | | | | | | | | |
| | 27904 | Template_14_0\|V326*T_D329*N_{—} | 7.9E−08 | 8.4E−08 | 18.3 | 0.0 | 3.7 | 0.1 | 5.0 | 0.4 | 7.48 |
| | | A330*S_T331*E_Y331*CT_{—} | | | | | | | | | |
| | | L331*ET_L331*GT | | | | | | | | | |
| | 27905 | Template_14_0\|V326*T_D329*N_{—} | 9.0E−08 | 9.1E−08 | 16.0 | 0.0 | 3.4 | 0.1 | 4.7 | 0.4 | 6.41 |
| | | A330*S_T331*N_Y331*CT_{—} | | | | | | | | | |
| | | L331*ET_L331*GT | | | | | | | | | |
| | 27906 | Template_14_0\|V326*T_D329*N_{—} | 8.1E−08 | 8.1E−08 | 17.9 | 0.0 | 3.8 | 0.1 | 4.7 | 0.4 | 6.72 |
| | | A330*A_T331*D_Y331*CT_{—} | | | | | | | | | |
| | | L331*ET_L331*GT | | | | | | | | | |
| | 27907 | Template_14_0\|V326*T_D329*N_{—} | 8.5E−08 | 8.2E−08 | 16.9 | 0.0 | 3.8 | 0.1 | 4.5 | 0.4 | 7.25 |
| | | A330*A_T331*E_Y331*CT_{—} | | | | | | | | | |
| | | L331*ET_L331*GT | | | | | | | | | |
| | 27908 | Template_14_0\|V326*T_D329*N_{—} | 1.0E−07 | 9.7E−08 | 14.3 | 0.0 | 3.2 | 0.1 | 4.5 | 0.4 | 7.24 |
| | | A330*A_T331*N_Y331*CT_{—} | | | | | | | | | |
| | | L331*ET_L331*GT | | | | | | | | | |
| | 27909 | Template_14_0\|V326*T_D329*S_{—} | 7.6E−08 | 7.7E−08 | 19.1 | 0.0 | 4.0 | 0.1 | 4.8 | 0.4 | 6.87 |
| | | A330*D_T331*D_Y331*CT_{—} | | | | | | | | | |
| | | L331*ET_L331*GT | | | | | | | | | |
| | 27910 | Template_14_0\|V326*T_D329*S_{—} | 7.7E−08 | 7.5E−08 | 18.9 | 0.0 | 4.1 | 0.1 | 4.6 | 0.4 | 7.77 |
| | | A330*D_T331*E_Y331*CT_{—} | | | | | | | | | |
| | | L331*ET_L331*GT | | | | | | | | | |
| | 27911 | Template_14_0\|V326*T_D329*S_{—} | 9.2E−08 | 8.7E−08 | 15.6 | 0.0 | 3.5 | 0.1 | 4.4 | 0.4 | 7.24 |
| | | A330*D_T331*N_Y331*CT_{—} | | | | | | | | | |
| | | L331*ET_L331*GT | | | | | | | | | |
| | 27912 | Template_14_0\|V326*T_D329*S_{—} | 8.0E−08 | 8.2E−08 | 18.2 | 0.0 | 3.8 | 0.1 | 4.8 | 0.4 | 7.10 |
| | | A330*D_T331*S_Y331*CT_{—} | | | | | | | | | |
| | | L331*ET_L331*GT | | | | | | | | | |
| | 27913 | Template_14_0\|V326*T_D329*S_{—} | 7.7E−08 | 8.0E−08 | 18.8 | 0.0 | 3.9 | 0.1 | 4.8 | 0.4 | 3.08 |
| | | A330*D_T331*T_Y331*CT_{—} | | | | | | | | | |
| | | L331*ET_L331*GT | | | | | | | | | |
| | 27914 | Template_14_0\|V326*T_D329*S_{—} | 7.5E−08 | 7.6E−08 | 19.3 | 0.0 | 4.1 | 0.1 | 4.8 | 0.4 | 7.94 |
| | | A330*E_T331*D_Y331*CT_{—} | | | | | | | | | |
| | | L331*ET_L331*GT | | | | | | | | | |
| | 27915 | Template_14_0\|V326*T_D329*S_{—} | 7.5E−08 | 7.2E−08 | 19.4 | 0.0 | 4.3 | 0.1 | 4.5 | 0.4 | 7.42 |
| | | A330*E_T331*E_Y331*CT_{—} | | | | | | | | | |
| | | L331*ET_L331*GT | | | | | | | | | |
| | 27916 | Template_14_0\|V326*T_D329*S_{—} | 9.3E−08 | 8.1E−08 | 15.5 | 0.0 | 3.8 | 0.1 | 4.1 | 0.3 | 5.27 |
| | | A330*E_T331*N_Y331*CT_{—} | | | | | | | | | |
| | | L331*ET_L331*GT | | | | | | | | | |
| | 27917 | Template_14_0\|V326*T_D329*S_{—} | 9.0E−08 | 8.5E−08 | 16.1 | 0.0 | 3.6 | 0.1 | 4.4 | 0.4 | 8.22 |
| | | A330*E_T331*S_Y331*CT_{—} | | | | | | | | | |
| | | L331*ET_L331*GT | | | | | | | | | |
| | 27918 | Template_14_0\|V326*T_D329*S_{—} | 8.8E−08 | 8.9E−08 | 16.4 | 0.0 | 3.5 | 0.1 | 4.7 | 0.4 | 7.58 |
| | | A330*E_T331*T_Y331*CT_{—} | | | | | | | | | |
| | | L331*ET_L331*GT | | | | | | | | | |
| | 27919 | Template_14_0\|V326*T_D329*S_{—} | 9.0E−08 | 8.4E−08 | 16.1 | 0.0 | 3.7 | 0.1 | 4.4 | 0.4 | 7.13 |
| | | A330*N_T331*D_Y331*CT_{—} | | | | | | | | | |
| | | L331*ET_L331*GT | | | | | | | | | |
| | 27920 | Template_14_0\|V326*T_D329*S_{—} | 8.2E−08 | 7.4E−08 | 17.6 | 0.0 | 4.2 | 0.1 | 4.2 | 0.4 | 7.84 |
| | | A330*N_T331*E_Y331*CT_{—} | | | | | | | | | |
| | | L331*ET_L331*GT | | | | | | | | | |
| | 27921 | Template_14_0\|V326*T_D329*S_{—} | 8.8E−08 | 8.9E−08 | 16.4 | 0.0 | 3.5 | 0.1 | 4.7 | 0.4 | 6.46 |
| | | A330*N_T331*N_Y331*CT_{—} | | | | | | | | | |
| | | L331*ET_L331*GT | | | | | | | | | |
| | 27922 | Template_14_0\|V326*T_D329*S_{—} | 8.7E−08 | 8.8E−08 | 16.6 | 0.0 | 3.5 | 0.1 | 4.7 | 0.4 | 6.78 |
| | | A330*N_T331*S_Y331*CT_{—} | | | | | | | | | |
| | | L331*ET_L331*GT | | | | | | | | | |
| | 27923 | Template_14_0\|V326*T_D329*S_{—} | 9.1E−08 | 8.6E−08 | 15.9 | 0.0 | 3.6 | 0.1 | 4.5 | 0.4 | 6.52 |
| | | A330*N_T331*T_Y331*CT_{—} | | | | | | | | | |
| | | L331*ET_L331*GT | | | | | | | | | |
| | 27924 | Template_14_0\|V326*T_D329*S_{—} | 7.8E−08 | 7.2E−08 | 18.5 | 0.0 | 4.3 | 0.1 | 4.3 | 0.4 | 55.15 |
| | | A330*S_T331*D_Y331*CT_{—} | | | | | | | | | |
| | | L331*ET_L331*GT | | | | | | | | | |
| | 27925 | Template_14_0\|V326*T_D329*S_{—} | 8.4E−08 | 8.2E−08 | 17.3 | 0.0 | 3.8 | 0.1 | 4.6 | 0.4 | 7.04 |
| | | A330*S_T331*E_Y331*CT_{—} | | | | | | | | | |
| | | L331*ET_L331*GT | | | | | | | | | |
| | 27926 | Template_14_0\|V326*T_D329*S_{—} | 1.0E−07 | 9.7E−08 | 14.0 | 0.0 | 3.2 | 0.1 | 4.4 | 0.4 | 6.63 |
| | | A330*S_T331*N_Y331*CT_{—} | | | | | | | | | |
| | | L331*ET_L331*GT | | | | | | | | | |
| | 27927 | Template_14_0\|V326*T_D329*S_{—} | 1.1E−07 | 9.9E−08 | 13.4 | 0.0 | 3.1 | 0.1 | 4.3 | 0.4 | 7.24 |
| | | A330*S_T331*S_Y331*CT_{—} | | | | | | | | | |
| | | L331*ET_L331*GT | | | | | | | | | |
| | 27928 | Template_14_0\|V326*T_D329*S_{—} | 8.9E−08 | 9.0E−08 | 16.3 | 0.0 | 3.4 | 0.1 | 4.8 | 0.4 | 7.64 |
| | | A330*S_T331*T_Y331*CT_{—} | | | | | | | | | |
| | | L331*ET_L331*GT | | | | | | | | | |
| | 27929 | Template_14_0\|V326*T_D329*S_{—} | 8.2E−08 | 8.1E−08 | 17.7 | 0.0 | 3.8 | 0.1 | 4.7 | 0.4 | 7.32 |
| | | A330*A_T331*D_Y331*CT_{—} | | | | | | | | | |
| | | L331*ET_L331*GT | | | | | | | | | |
| | 27930 | Template_14_0\|V326*T_D329*S_{—} | 7.7E−08 | 8.0E−08 | 18.7 | 0.0 | 3.8 | 0.1 | 4.9 | 0.4 | 7.53 |
| | | A330*A_T331*E_Y331*CT_{—} | | | | | | | | | |
| | | L331*ET_L331*GT | | | | | | | | | |
| | 27931 | Template_14_0\|V326*T_D329*S_{—} | 1.0E−07 | 9.3E−08 | 13.9 | 0.0 | 3.3 | 0.1 | 4.2 | 0.4 | 7.16 |
| | | A330*A_T331*N_Y331*CT_{—} | | | | | | | | | |
| | | L331*ET_L331*GT | | | | | | | | | |
| | 27932 | Template_14_0\|V326*T_D329*S_{—} | 1.0E−07 | 9.3E−08 | 14.4 | 0.0 | 3.3 | 0.1 | 4.4 | 0.4 | −1.87 |
| | | A330*A_T331*S_Y331*CT_{—} | | | | | | | | | |
| | | L331*ET_L331*GT | | | | | | | | | |
| | 27933 | Template_14_0\|V326*T_D329*S_{—} | 1.0E−07 | 8.9E−08 | 14.4 | 0.0 | 3.5 | 0.1 | 4.2 | 0.4 | 7.39 |
| | | A330*A_T331*T_Y331*CT_{—} | | | | | | | | | |
| | | L331*ET_L331*GT | | | | | | | | | |
| Strat4 | 27499 | Template_11_14 | 3.9E−08 | 4.9E−08 | 37.3 | 0.1 | 6.3 | 0.2 | 5.9 | 0.5 | 13.64 |
| Template_11_14 | 27500 | Template_cl_4372-11_14\|P326*P_{—} | ND | ND | — | — | — | — | — | — | 2.17 |
| | | F331*DF | | | | | | | | | |
| | 27504 | Template_11_14\| P326*T_F331*DT | 9.2E−08 | 1.2E−07 | 15.6 | 0.0 | 2.6 | 0.1 | 6.0 | 0.5 | 13.67 |
| | 27534 | Template_11_14\|V266I_P326*T_{—} | 8.2E−08 | 1.2E−07 | 17.5 | 0.0 | 2.6 | 0.1 | 6.7 | 0.6 | 18.42 |
| | | F331*DT | | | | | | | | | |
| | 27535 | Template_11_14\|V266L_P326*T_{—} | 1.1E−07 | 1.7E−07 | 13.0 | 0.0 | 1.8 | 0.1 | 7.3 | 0.6 | 28.54 |
| | | F331*DT | | | | | | | | | |
| | 27536 | Template_11_14\|V266F_P326*T_{—} | ND | ND | — | — | — | — | — | — | 1.87 |
| | | F331*DT | | | | | | | | | |
| | 27537 | Template_11_14\|V273I_P326*T_{—} | 4.6E−08 | 5.4E−08 | 31.4 | 0.1 | 5.7 | 0.2 | 5.5 | 0.5 | 5.78 |
| | | F331*DT | | | | | | | | | |
| | 27538 | Template_11_14\|V273L_P326*T_{—} | 3.8E−08 | 3.8E−08 | 37.7 | 0.1 | 8.2 | 0.2 | 4.6 | 0.4 | 4.67 |
| | | F331*DT | | | | | | | | | |
| | 27539 | Template_11_14\|V273F_P326*T_{—} | 2.9E−08 | 2.7E−08 | 49.2 | 0.1 | 11.5 | 0.3 | 4.3 | 0.4 | 25.49 |
| | | F331*DT | | | | | | | | | |
| | 27540 | Template_11_14\|A325*V_P326*T_{—} | 7.9E−08 | 1.4E−07 | 18.4 | 0.0 | 2.2 | 0.1 | 8.3 | 0.7 | 25.90 |
| | | F331*DT | | | | | | | | | |
| | 27541 | Template_11_14\|A325*I_P326*T_{—} | 7.1E−08 | 1.0E−07 | 20.2 | 0.0 | 3.1 | 0.1 | 6.6 | 0.6 | 16.72 |
| | | F331*DT | | | | | | | | | |
| | 27542 | Template_11_14\|A325*L_P326*T_{—} | 3.4E−08 | 4.8E−08 | 42.4 | 0.1 | 6.5 | 0.2 | 6.6 | 0.6 | 14.82 |
| | | F331*DT | | | | | | | | | |
| | 27543 | Template_11_14\|A325*F_P326*T_{—} | 5.8E−08 | 8.4E−08 | 24.8 | 0.1 | 3.7 | 0.1 | 6.8 | 0.6 | 15.61 |
| | | F331*DT | | | | | | | | | |
| | 27934 | Template_11_14\| P326*T_N329*D_{—} | 6.9E−08 | 8.5E−08 | 20.8 | 0.1 | 3.6 | 0.1 | 5.8 | 0.5 | 12.82 |
| | | H331*D_F331*DT | | | | | | | | | |
| | 27935 | Template_11_14\| P326*T_N329*D_{—} | 7.3E−08 | 8.8E−08 | 19.7 | 0.0 | 3.5 | 0.1 | 5.6 | 0.5 | 12.66 |
| | | H331*E_F331*DT | | | | | | | | | |
| | 27936 | Template_11_14\| P326*T_N329*D_{—} | 7.1E−08 | 9.4E−08 | 20.2 | 0.0 | 3.3 | 0.1 | 6.2 | 0.5 | 14.23 |
| | | H331*N_F331*DT | | | | | | | | | |
| | 27937 | Template_11_14\| P326*T_N329*D_{—} | 6.1E−08 | 8.2E−08 | 23.7 | 0.1 | 3.8 | 0.1 | 6.3 | 0.5 | |
| | | H331*S_F331*DT | | | | | | | | | |
| | 27938 | Template_11_14\| P326*T_N329*D_{—} | 7.3E−08 | 9.8E−08 | 19.7 | 0.0 | 3.1 | 0.1 | 6.3 | 0.5 | 15.12 |
| | | H331*H_F331*DT | | | | | | | | | |
| | 27939 | Template_11_14\| P326*T_N329*E_{—} | 6.2E−08 | 7.9E−08 | 23.2 | 0.1 | 3.9 | 0.1 | 5.9 | 0.5 | 12.52 |
| | | H331*D_F331*DT | | | | | | | | | |
| | 27940 | Template_11_14\| P326*T_N329*E_{—} | 6.3E−08 | 7.3E−08 | 23.0 | 0.1 | 4.2 | 0.1 | 5.5 | 0.5 | 12.80 |
| | | H331*E_F331*DT | | | | | | | | | |
| | 27941 | Template_11_14\| P326*T_N329*E_{—} | 5.7E−08 | 7.9E−08 | 25.6 | 0.1 | 3.9 | 0.1 | 6.6 | 0.6 | |
| | | H331*N_F331*DT | | | | | | | | | |
| | 27942 | Template_11_14\| P326*T_N329*E_{—} | 8.1E−08 | 1.0E−07 | 17.9 | 0.0 | 3.0 | 0.1 | 5.9 | 0.5 | 11.84 |
| | | H331*S_F331*DT | | | | | | | | | |
| | 27943 | Template_11_14\| P326*T_N329*E_{—} | 9.5E−08 | 1.2E−07 | 15.3 | 0.0 | 2.6 | 0.1 | 5.9 | 0.5 | 13.98 |
| | | H331*H_F331*DT | | | | | | | | | |
| | 27944 | Template_11_14\| P326*T_N329*N_{—} | 7.1E−08 | 9.2E−08 | 20.4 | 0.0 | 3.4 | 0.1 | 6.1 | 0.5 | 15.34 |
| | | H331*D_F331*DT | | | | | | | | | |
| | 27945 | Template_11_14\| P326*T_N329*N_{—} | 6.8E−08 | 9.0E−08 | 21.3 | 0.1 | 3.4 | 0.1 | 6.2 | 0.5 | 6.37 |
| | | H331*E_F331*DT | | | | | | | | | |
| | 27946 | Template_11_14\| P326*T_N329*N_{—} | 8.5E−08 | 1.1E−07 | 17.0 | 0.0 | 2.7 | 0.1 | 6.3 | 0.5 | 10.39 |
| | | H331*N_F331*DT | | | | | | | | | |
| | 27947 | Template_11_14\| P326*T_N329*N_{—} | 8.3E−08 | 1.1E−07 | 17.5 | 0.0 | 2.8 | 0.1 | 6.3 | 0.5 | 17.50 |
| | | H331*S_F331*DT | | | | | | | | | |
| | 27948 | Template_11_14\| P326*T_N329*S_{—} | 7.9E−08 | 8.7E−08 | 18.2 | 0.0 | 3.5 | 0.1 | 5.2 | 0.4 | 6.17 |
| | | H331*D_F331*DT | | | | | | | | | |
| | 27949 | Template_11_14\| P326*T_N329*S_{—} | 7.4E−08 | 8.8E−08 | 19.5 | 0.0 | 3.5 | 0.1 | 5.6 | 0.5 | 13.84 |
| | | H331*E_F331*DT | | | | | | | | | |
| | 27950 | Template_11_14\| P326*T_N329*S_{—} | 1.0E−07 | 1.2E−07 | 14.4 | 0.0 | 2.5 | 0.1 | 5.8 | 0.5 | 15.53 |
| | | H331*N_F331*DT | | | | | | | | | |
| | 27951 | Template_11_14\| P326*T_N329*S_{—} | 9.9E−08 | 1.2E−07 | 14.6 | 0.0 | 2.5 | 0.1 | 5.8 | 0.5 | 15.36 |
| | | H331*S_F331*DT | | | | | | | | | |
| | 27952 | Template_11_14\| P326*T_N329*S_{—} | 9.9E−08 | 1.2E−07 | 14.6 | 0.0 | 2.6 | 0.1 | 5.7 | 0.5 | 15.33 |
| | | H331*H_F331*DT | | | | | | | | | |
| | 27953 | Template_11_14\| P326*T_N329*H_{—} | 7.3E−08 | 1.0E−07 | 19.7 | 0.0 | 3.1 | 0.1 | 6.4 | 0.5 | 14.10 |
| | | H331*D_F331*DT | | | | | | | | | |
| | 27954 | Template_11_14\| P326*T_N329*H_{—} | 7.4E−08 | 9.9E−08 | 19.4 | 0.0 | 3.1 | 0.1 | 6.2 | 0.5 | 14.71 |
| | | H331*E_F331*DT | | | | | | | | | |
| | 27955 | Template_11_14\| P326*T_N329*H_{—} | 9.7E−08 | 1.3E−07 | 14.9 | 0.0 | 2.4 | 0.1 | 6.2 | 0.5 | 16.51 |
| | | H331*N_F331*DT | | | | | | | | | |
| | 27956 | Template_11_14\| P326*T_N329*H_{—} | 1.0E−07 | 1.2E−07 | 14.5 | 0.0 | 2.6 | 0.1 | 5.6 | 0.5 | 15.42 |
| | | H331*S_F331*DT | | | | | | | | | |
| | 27957 | Template_11_14\| P326*T_N329*H_{—} | 1.2E−07 | 1.6E−07 | 12.1 | 0.0 | 1.9 | 0.1 | 6.3 | 0.5 | 18.22 |
| | | H331*H_F331*DT | | | | | | | | | |

¹All Strategy 4 variants include the core mutations: A_G236N_G237A/B_G236D_G237F_S239D_S267V_H268D with the noted loop Template sequence replacing residues 325-331 in Chain B, with the exception of those variants designated “cl_4372” (v27494, v27496, v27498 & v27500), which contain the wild-type IgG1 CH2 sequence.
Loop Template sequences are as follows: Template_13_3: VLDDPSRENEADL [SEQ ID NO: 16]; Template_12_14: NFTPKAKLGFEI [SEQ ID NO: 17]; Template_12_14-2: DFTPKAKLGFQI [SEQ ID NO: 182]; Template_14_0: QVHEDATKPYGLSL [SEQ ID NO: 18]; Template_11_14: APQINPHSPKF [SEQ ID NO: 19]
²Mutation notation is in the format Template_X|P326*T, where “Template_X” indicates the parental loop Template sequence and “P326*T” indicates the mutation made with “P” representing the parental residue being replaced, 326* representing the position and T representing the replacement residue.
³Selectivity is defined as IIb-Fold / IIaR-Fold
⁴% of non-competed FcγRIIb signal in presence of 10× FcγRIIa
⁵IIb-Specific Comparator: Mimoto, et al., 2013, Protein Eng. Des. Sel., 26: 589-598
⁶ND—signal too low for accurate measurement

TABLE 6.21
Strategy 5 Variants

| | | | | | | | | | | IIb | |
|---|---|---|---|---|---|---|---|---|---|---|---|
| | | | | | | | | | | Selec- | |
| | | | | | | IIb- | | IIaR- | | tivity² | |
| | | | | | | Fold | | Fold | IIb | Fold | |
| | | | FcγRIIb | FcγRIIaR | IIb- | wrt | IIaR- | wrt | Selec- | wrt | |
| Strategy | Variant # | Mutations¹ | K_{D} | K_{D} | Fold | Control | Fold | Control | tivity² | Control | ELISA³ |
| Controls | 16463 | WT | 1.4E−06 | 3.1E−07 | 1.0 | | 1.0 | | 1.0 | | |
| | 27293 | strat1_control | 3.5E−09 | 8.8E−09 | 415.4 | 1.0 | 35.2 | 1.0 | 11.8 | 1.0 | 70.76 |
| | | (A_G236N_G237A | | | | | | | | | |
| | | B_G236D_G237F_{—} | | | | | | | | | |
| | | S239D_S267V_{—} | | | | | | | | | |
| | | H268D_Template_1) | | | | | | | | | |
| | 28472 | strat1_control + E269K | 5.0E−09 | 1.8E−08 | 290.7 | 0.7 | 17.0 | 0.5 | 17.1 | 1.4 | 59.75 |
| | 27294 | strat2_control | 1.6E−08 | 4.4E−08 | 92.9 | 1.0 | 7.0 | 1.0 | 13.4 | 1.0 | 47.77 |
| | | (A_L234F_G236N_{—} | | | | | | | | | |
| | | H268Q_A327G_A330K_P331S | | | | | | | | | |
| | | B_G236D_S239D_{—} | | | | | | | | | |
| | | V266L_S267A_H268D) | | | | | | | | | |
| | v12⁴ | Symmetrical | 1.3E−08 | 2.2E−07 | 111.6 | | 1.4 | | 80.5 | | 100.12 |
| | | E233D_G237D_P238D_{—} | | | | | | | | | |
| | | H268D_P271G_A330R | | | | | | | | | |
| Strat5 | 27296 | LM1_A287F_strat5 | 3.1E−09 | 1.1E−08 | 469.2 | 1.1 | 28.6 | 0.8 | 16.4 | 1.4 | 59.13 |
| LM1 | 27298 | LM1_M428F_strat5 | 3.3E−09 | 1.2E−08 | 436.6 | 1.1 | 26.4 | 0.7 | 16.6 | 1.4 | 68.84 |
| | 27300 | LM1_T250V_strat5 | 3.0E−09 | 1.1E−08 | 478.5 | 1.2 | 27.8 | 0.8 | 17.2 | 1.5 | 44.87 |
| | 27302 | LM1_L309Q_strat5 | 2.8E−09 | 1.1E−08 | 512.1 | 1.2 | 28.4 | 0.8 | 18.1 | 1.5 | |
| | 27304 | LM1_L242C_I336C_strat5 | ND⁵ | ND | — | — | — | — | — | — | −12.31 |
| | 27306 | LM1_V308I_strat5 | 3.5E−09 | 1.6E−08 | 416.5 | 1.0 | 19.8 | 0.6 | 21.1 | 1.8 | 37.28 |
| | 27308 | LM1_A287F_M428F_strat5 | 3.1E−09 | 1.1E−08 | 464.7 | 1.1 | 29.1 | 0.8 | 16.0 | 1.4 | 61.63 |
| | 27310 | LM1_A287F_T250V_strat5 | 2.0E−09 | 8.8E−09 | 732.8 | 1.8 | 35.2 | 1.0 | 20.8 | 1.8 | |
| | 27312 | LM1_M428F_T250V_strat5 | 4.1E−09 | 1.4E−08 | 349.1 | 0.8 | 22.5 | 0.6 | 15.5 | 1.3 | 84.33 |
| | 27314 | LM1_A287F_M428F_{—} | ND | ND | — | — | — | — | — | — | −31.02 |
| | | T250V_strat5 | | | | | | | | | |
| | 27316 | LM1_T250V_L309Q_strat5 | 3.9E−09 | 1.4E−08 | 373.4 | 0.9 | 22.2 | 0.6 | 16.8 | 1.4 | 62.06 |
| | 27318 | LM1_L242C_I336C_{—} | 3.6E−09 | 1.2E−08 | 402.6 | 1.0 | 24.9 | 0.7 | 16.2 | 1.4 | 28.43 |
| | | V308I_strat5 | | | | | | | | | |
| Strat5 | 27297 | LM2_A287F_strat5 | 2.0E−08 | 6.2E−08 | 71.2 | 0.2 | 4.9 | 0.1 | 14.4 | 1.2 | 54.36 |
| LM2 | 27299 | LM2_M428F_strat5 | 1.6E−08 | 4.9E−08 | 91.5 | 0.2 | 6.3 | 0.2 | 14.5 | 1.2 | 57.11 |
| | 27301 | LM2_T250V_strat5 | 1.7E−08 | 5.6E−08 | 84.5 | 0.2 | 5.5 | 0.2 | 15.3 | 1.3 | 48.30 |
| | 27303 | LM2_L309Q_strat5 | 2.1E−08 | 6.0E−08 | 69.5 | 0.2 | 5.1 | 0.1 | 13.5 | 1.1 | 58.28 |
| | 27305 | LM2_L242C_I336C_strat5 | 1.9E−08 | 6.6E−08 | 74.5 | 0.2 | 4.7 | 0.1 | 15.9 | 1.3 | 63.96 |
| | 27307 | LM2_V308I_strat5 | 2.0E−08 | 5.9E−08 | 74.1 | 0.2 | 5.2 | 0.1 | 14.3 | 1.2 | 64.25 |
| | 27309 | LM2_A287F_M428F_strat5 | 1.9E−08 | 5.8E−08 | 75.7 | 0.2 | 5.3 | 0.2 | 14.3 | 1.2 | 56.27 |
| | 27311 | LM2_A287F_T250V_strat5 | 1.7E−08 | 5.9E−08 | 85.0 | 0.2 | 5.2 | 0.1 | 16.3 | 1.4 | 66.71 |
| | 27313 | LM2_M428F_T250V_strat5 | 1.8E−08 | 5.3E−08 | 79.0 | 0.2 | 5.8 | 0.2 | 13.5 | 1.1 | 46.69 |
| | 27315 | LM2_A287F_M428F_{—} | ND | ND | — | — | — | — | — | — | −12.46 |
| | | T250V_strat5 | | | | | | | | | |
| | 27317 | LM2_T250V_L309Q_strat5 | 1.9E−08 | 5.6E−08 | 77.7 | 0.2 | 5.5 | 0.2 | 14.2 | 1.2 | 67.19 |
| | 27319 | LM2_L242C_I336C_{—} | 1.6E−08 | 4.3E−08 | 92.6 | 0.2 | 7.2 | 0.2 | 12.9 | 1.1 | |
| | | V308I_strat5 | | | | | | | | | |

¹The noted stability-enhancing mutations were added symmetrically into a background of “LM1” (Launching Module 1) mutations (A_G236N_G237A/B_G236D_G237F_S239D_S267V_H268D_Template_1) or “LM2” (Launching Module 2) mutations (A_L234F_G236N_H268Q_A327G_A330K_P331S/B_G236D_S239D_V266L_S267A_H268D) as noted.
²Selectivity is defined as IIb-Fold/IIaR-Fold
³% of non-competed FcyRIIb signal in presence of 10x FcyRIIa
⁴IIb-Specific Comparator: Mimoto, et al., 2013, Protein Eng. Des. Sel., 26: 589-598
⁵ND - signal too low for accurate measurement

TABLE 6.22
Strategy 1 Variants Meeting Criteria A

| | | | | | IIb- | | IIb Selectivity |
|---|---|---|---|---|---|---|---|
| | | | | | Fold | | Improvement |
| | | | IIb- | IIaR- | wrt | IIb | wrt |
| Position | Mutations¹ | Variant # | Fold | Fold | Control | Selectivity | Control |
| Control | Strat1 control | 27293 | 415 | 35 | 1.0 | 12 | 1.0 |
| A_234 | A_L234D_strat1 | 26105 | 636 | 29 | 1.53 | 22 | 1.9 |
| | A_L234F_strat1 | 26098 | 547 | 28 | 1.32 | 19 | 1.6 |
| | A_L234Q_strat1 | 26103 | 396 | 20 | 0.95 | 20 | 1.7 |
| | A_L234T_strat1 | 26101 | 443 | 24 | 1.07 | 18 | 1.6 |
| | A_L234W_strat1 | 26099 | 1,132 | 53 | 2.72 | 21 | 1.8 |
| A_235 | A_L235A_strat1 | 26112 | 285 | 15 | 0.68 | 19 | 1.6 |
| | A_L235D_strat1 | 26123 | 457 | 23 | 1.10 | 20 | 1.7 |
| | A_L235E_strat1 | 26124 | 466 | 25 | 1.12 | 19 | 1.6 |
| | A_L235F_strat1 | 26116 | 1,323 | 53 | 3.19 | 25 | 2.1 |
| | A_L235H_strat1 | 26127 | 270 | 15 | 0.65 | 18 | 1.5 |
| | A_L235R_strat1 | 26125 | 149 | 8 | 0.36 | 19 | 1.6 |
| | A_L235W_strat1 | 26117 | 1,690 | 70 | 4.07 | 24 | 2.1 |
| | A_L235Y_strat1 | 26118 | 1,167 | 53 | 2.81 | 22 | 1.9 |
| A_237 | A_A237D_strat1 | 26159 | 185 | 7 | 0.45 | 26 | 2.2 |
| | A_A237L_strat1 | 26149 | 252 | 12 | 0.61 | 20 | 1.7 |
| | A_A237N_strat1 | 26158 | 307 | 16 | 0.74 | 19 | 1.6 |
| A_239 | A_S239A_strat1 | 26166 | 725 | 35 | 1.75 | 21 | 1.8 |
| | A_S239G_strat1 | 26165 | 639 | 29 | 1.54 | 22 | 1.9 |
| | A_S239H_strat1 | 26181 | 464 | 22 | 1.12 | 21 | 1.8 |
| | A_S239T_strat1 | 26174 | 765 | 37 | 1.84 | 21 | 1.8 |
| | A_S239Y_strat1 | 26173 | 575 | 26 | 1.38 | 22 | 1.9 |
| B_236 | B_D236K_strat1 | 26235 | 346 | 17 | 0.83 | 20 | 1.7 |
| B_271 | B_P271D_strat1 | 26381 | 162 | 8 | 0.39 | 20 | 1.7 |
| B_323 | B_V323A_strat1 | 26392 | 488 | 25 | 1.17 | 20 | 1.7 |
| B_325* | B_S325*A_strat1 | 26399 | 487 | 27 | 1.17 | 18 | 1.5 |
| B_326* | B_T326*A_strat1 | 26417 | 337 | 19 | 0.81 | 18 | 1.5 |
| | B_T326*D_strat1 | 26428 | 1,120 | 55 | 2.70 | 21 | 1.7 |
| | B_T326*E_strat1 | 26429 | 817 | 39 | 1.97 | 21 | 1.8 |
| | B_T326*F_strat1 | 26422 | 465 | 26 | 1.12 | 18 | 1.5 |
| | B_T326*H_strat1 | 26432 | 508 | 27 | 1.22 | 19 | 1.6 |
| | B_T326*I_strat1 | 26420 | 468 | 26 | 1.13 | 18 | 1.5 |
| | B_T326*L_strat1 | 26419 | 382 | 19 | 0.92 | 20 | 1.7 |
| | B_T326*N_strat1 | 26427 | 757 | 41 | 1.82 | 19 | 1.6 |
| | B_T326*Q_strat1 | 26426 | 412 | 21 | 0.99 | 19 | 1.6 |
| | B_T326*V_strat1 | 26418 | 462 | 24 | 1.1 | 19 | 1.6 |
| | B_T326*W_strat1 | 26423 | 416 | 22 | 1.00 | 19 | 1.6 |
| B_328* | B_F328*H_strat1 | 26468 | 290 | 15 | 0.70 | 20 | 1.7 |
| | B_F328*S_strat1 | 26461 | 250 | 13 | 0.60 | 19 | 1.6 |
| | B_F328*Y_strat1 | 26459 | 463 | 25 | 1.11 | 18 | 1.6 |
| B_329* | B_D329*G_strat1 | 26470 | 1,039 | 58 | 2.50 | 18 | 1.5 |
| | B_D329*I_strat1 | 26474 | 259 | 4 | 0.62 | 58 | 4.9 |
| | B_D329*L_strat1 | 26473 | 350 | 9 | 0.84 | 37 | 3.1 |
| B_330* | B_G330*A_strat1 | 26488 | 2,829 | 128 | 6.81 | 22 | 1.9 |
| | B_G330*D_strat1 | 26500 | 282 | 10 | 0.68 | 29 | 2.5 |
| | B_G330*E_strat1 | 26501 | 857 | 45 | 2.06 | 19 | 1.6 |
| | B_G330*H_strat1 | 26504 | 1,571 | 61 | 3.78 | 26 | 2.2 |
| | B_G330*K_strat1 | 26503 | 1,025 | 27 | 2.47 | 39 | 3.3 |
| | B_G330*R_strat1 | 26502 | 185 | 9 | 0.45 | 20 | 1.7 |
| B_331* | B_Y331*AF_strat1 | 26530 | 489 | 28 | 1.18 | 18 | 1.5 |
| A | B_Y331*AW_strat1 | 26531 | 411 | 21 | 0.99 | 20 | 1.7 |
| B_331*B | B_A331*BF_strat1 | 26546 | 182 | 10 | 0.44 | 18 | 1.5 |
| | B_A331*BH_strat1 | 26557 | 414 | 22 | 1.00 | 19 | 1.6 |
| | B_A331*BK_strat1 | 26556 | 632 | 33 | 1.52 | 19 | 1.6 |
| | B_A331*BL_strat1 | 26543 | 596 | 25 | 1.44 | 24 | 2.0 |
| B_332 | B_I332F_strat1 | 26563 | 408 | 23 | 0.98 | 18 | 1.5 |
| | B_I332L_strat1 | 26561 | 911 | 33 | 2.19 | 28 | 2.3 |

¹Mutation notation is in the format A_L234G_strat1, where “A” indicates the Fc chain, “L234G” indicates the mutation made with “L” representing the parental residue being replaced, 234 representing the position and G representing the replacement residue, and “strat1” specifies the parental CH2 mutations (A_G236N_G237A/B_G236D_G237F_S239D_S267V_H268D_Template1)

TABLE 6.23
Strategy 2 Variants Meeting Criteria A

| | | | | IIb- | | | IIb Selectivity |
|---|---|---|---|---|---|---|---|
| | | | | Fold | | | Improvement |
| | | | IIb- | wrt | IIaR- | IIb | wrt |
| Position | Mutations¹ | Variant # | Fold | Control | fold | Selectivity | Control |
| A_235 | A_L235D_strat2 | 26593 | 65.2 | 0.7 | 2.5 | 26.3 | 2.0 |
| A_267 | A_S267A_strat2 | 26663 | 62.1 | 0.7 | 2.6 | 23.6 | 1.8 |
| A_330 | A_K330T_strat2 | 26847 | 53.3 | 0.6 | 2.4 | 22.6 | 1.7 |
| B_237 | B_G237D_strat2 | 26940 | 304.2 | 3.3 | 13.1 | 23.3 | 1.7 |
| | B_G237L_strat2 | 26931 | 296.1 | 3.2 | 14.2 | 20.9 | 1.6 |
| | B_G237N_strat2 | 26939 | 541.7 | 5.8 | 27.5 | 19.7 | 1.5 |

¹Mutation notation is in the format A_F234G_strat2, where “A” indicates the Fc chain, “F234G” indicates the mutation made with “F” representing the parental residue being replaced, 234 representing the position and G representing the replacement residue, and “strat2” specifies the parental CH2 mutations (A_L234F_G236N_H268Q_A327G_A330K_P331S/B_G236D_S239D_V266L_S267A_H268D)

TABLE 6.24
Strategy 3 Variants Meeting Criteria A

| | | | | | IIb- | | IIb Selectivity |
|---|---|---|---|---|---|---|---|
| | | | | | Fold | | Improvement |
| Loop | | | IIb- | IIaR- | wrt | IIb | wrt |
| Template | Mutations¹ | Variant # | Fold | Fold | Control | Selectivity | Control |
| Template | template1_S325*A_A331*BN_strat3 | 27374 | 332.1 | 21.2 | 35.2 | 15.7 | 1.8 |
| 1 | template1_T326*H_W327*W_F328*D_D329*D_strat3 | 27372 | 92.2 | 6.4 | 9.8 | 14.4 | 1.6 |
| | template1_T326*H_W327*W_F328*E_D329*G_A331*BN_{—} | 27383 | 608.5 | 28.1 | 64.5 | 21.7 | 2.5 |
| | strat3 | | | | | | |
| | template1_T326*H_W327*W_F328*E_D329*G_S325*A_{—} | 27389 | 505.1 | 28.9 | 53.6 | 17.5 | 2.0 |
| | A331*BN_strat3 | | | | | | |
| | template1_T326*H_W327*W_F328*E_D329*G_strat3 | 27365 | 838.3 | 44.8 | 88.9 | 18.7 | 2.1 |
| | template1_T326*H_W327*W_F328*F_D329*D_A331*BN_{—} | 27385 | 337.3 | 20.9 | 35.8 | 16.1 | 1.8 |
| | strat3 | | | | | | |
| | template1_T326*H_W327*W_F328*F_D329*D_S325*A_{—} | 27391 | 402.4 | 26.8 | 42.7 | 15.0 | 1.7 |
| | A331*BN_strat3 | | | | | | |
| | template1_T326*H_W327*W_F328*F_D329*D_S325*A_{—} | 27379 | 486.3 | 32.0 | 51.6 | 15.2 | 1.7 |
| | strat3 | | | | | | |
| | template1_T326*H_W327*W_F328*H_D329*D_strat3 | 27373 | 332.1 | 17.4 | 35.2 | 19.1 | 2.2 |
| | template1_T326*H_W327*W_F328*H_D329*G_strat3 | 27393 | 393.0 | 21.3 | 41.7 | 18.4 | 2.1 |
| | template1_T326*H_W327*W_F328*N_D329*D_strat3 | 27367 | 148.7 | 8.7 | 15.8 | 17.2 | 2.0 |
| | template1_T326*H_W327*W_F328*Q_D329*D_strat3 | 27368 | 187.9 | 13.2 | 19.9 | 14.3 | 1.6 |
| | template1_T326*H_W327*W_F328*Q_D329*G_A331*BN_{—} | 27384 | 255.9 | 15.5 | 27.1 | 16.5 | 1.9 |
| | strat3 | | | | | | |
| | template1_T326*H_W327*W_F328*Q_D329*G_S325*A_{—} | 27390 | 247.9 | 16.0 | 26.3 | 15.5 | 1.8 |
| | A331*BN_strat3 | | | | | | |
| | template1_T326*H_W327*W_F328*Q_D329*G_S325*A_{—} | 27378 | 322.7 | 19.7 | 34.2 | 16.4 | 1.9 |
| | strat3 | | | | | | |
| | template1_T326*H_W327*W_F328*Q_D329*G_strat3 | 27366 | 450.1 | 22.5 | 47.7 | 20.0 | 2.3 |
| | template1_T326*H_W327*W_F328*S_D329*D_A331*BN_{—} | 27381 | 172.3 | 12.2 | 18.3 | 14.2 | 1.6 |
| | strat3 | | | | | | |
| | template1_T326*H_W327*W_F328*S_D329*D_S325*A_{—} | 27387 | 108.4 | 8.1 | 11.5 | 13.4 | 1.5 |
| | A331*BN_strat3 | | | | | | |
| | template1_T326*H_W327*W_F328*S_D329*D_S325*A_{—} | 27375 | 164.8 | 10.2 | 17.5 | 16.2 | 1.9 |
| | strat3 | | | | | | |
| | template1_T326*H_W327*W_F328*S_D329*D_strat3 | 27363 | 188.4 | 10.9 | 20.0 | 17.2 | 2.0 |
| | template1_T326*H_W327*W_F328*T_D329*D_strat3 | 27371 | 237.0 | 16.7 | 25.1 | 14.2 | 1.6 |
| | template1_T326*T_W327*W_F328*H_D329*G_strat3 | 27394 | 429.6 | 22.6 | 45.6 | 19.0 | 2.2 |
| | template1_T326*T_W327*W_F328*Q_D329*G_strat3 | 27369 | 386.3 | 18.7 | 41.0 | 20.6 | 2.4 |
| | template1_T326*T_W327*W_F328*S_D329*D_A331*BN_{—} | 27386 | 138.2 | 9.3 | 14.7 | 14.8 | 1.7 |
| | strat3 | | | | | | |
| | template1_T326*T_W327*W_F328*S_D329*D_S325*A_{—} | 27392 | 74.5 | 5.5 | 7.9 | 13.5 | 1.5 |
| | A331*BN_strat3 | | | | | | |
| | template1_T326*T_W327*W_F328*S_D329*G_strat3 | 27370 | 296.5 | 19.4 | 31.5 | 15.3 | 1.8 |
| Template | template7_E328*E_E329*N_A331*BV_G325*F_strat3 | 27461 | 63.5 | 4.4 | 6.7 | 14.4 | 1.6 |
| 7 | template7_E328*E_E329*N_A331*BY_strat3 | 27453 | 107.9 | 7.1 | 11.4 | 15.3 | 1.7 |
| | template7_E328*H_E329*R_A331*BV_G325*F_strat3 | 27463 | 34.9 | 2.6 | 3.7 | 13.5 | 1.5 |
| | template7_E328*H_E329*R_A331*BY_strat3 | 27455 | 86.7 | 2.8 | 9.2 | 30.5 | 3.5 |
| | template7_E328*Q_E329*S_A331*BV_G325*F_strat3 | 27464 | 56.4 | 4.3 | 6.0 | 13.2 | 1.5 |
| | template7_E328*Q_E329*S_A331*BY_strat3 | 27456 | 59.7 | 4.0 | 6.3 | 15.0 | 1.7 |
| | template7_E328*T_E329*N_A331*BV_G325*F_strat3 | 27462 | 55.8 | 4.2 | 5.9 | 13.1 | 1.5 |
| | template7_E328*T_E329*N_A331*BY_strat3 | 27454 | 55.6 | 4.0 | 5.9 | 13.9 | 1.6 |
| Template | template66_D327*D_Q328*E_N329*D_Q330*D_strat3 | 27401 | 58.2 | 4.2 | 6.2 | 13.9 | 1.6 |
| 66 | template66_D327*D_Q328*H_N329*D_Q330*Q_strat3 | 27403 | 47.8 | 3.0 | 5.1 | 16.1 | 1.8 |
| | template66_D327*D_Q328*N_N329*D_Q330*D_strat3 | 27405 | 75.3 | 4.4 | 8.0 | 17.2 | 2.0 |
| | template66_D327*D_Q328*S_N329*T_Q330*D_strat3 | 27404 | 57.6 | 3.3 | 6.1 | 17.7 | 2.0 |
| | template66_D327*D_Q328*S_N329*T_Q330*Q_strat3 | 27408 | 52.5 | 3.2 | 5.6 | 16.6 | 1.9 |
| | template66_D327*D_Q328*T_N329*D_Q330*D_strat3 | 27406 | 64.9 | 3.7 | 6.9 | 17.5 | 2.0 |
| | template66_D327*D_Q328*T_N329*S_Q330*Q_strat3 | 27410 | 48.8 | 2.9 | 5.2 | 16.6 | 1.9 |
| | template66_D327*N_Q328*H_N329*N_Q330*D_strat3 | 27411 | 26.5 | 1.9 | 2.8 | 13.6 | 1.6 |
| Template | template151_E328*E_E329*D_Y331*BI_R331*CS_strat3 | 27474 | 163.5 | 10.0 | 17.3 | 16.3 | 1.9 |
| 151 | template151_E328*E_E329*D_Y331*BI_strat3 | 27472 | 144.1 | 9.5 | 15.3 | 15.2 | 1.7 |
| | template151_E328*H_E329*N_Y331*BI_strat3 | 27471 | 43.9 | 2.3 | 4.7 | 19.4 | 2.2 |
| | template151_Y331*BI_strat3 | 27466 | 128.8 | 8.6 | 13.7 | 15.0 | 1.7 |

¹Mutation notation is in the format “template1_T326*H_strat3,” where “template1” indicates the parental loop template, “T326*H” indicates the mutation made with “T” representing the parental residue being replaced, 326* representing the position and H representing the replacement residue, and “strat3” specifies the parental CH2 mutations (A_G236N_G237A/B_G236D_G237F_S239D_S267V_H268D).

TABLE 6.25
Strategy 1 Variants Meeting Criteria B

| | | | | | IIb- | | IIb Selectivity |
|---|---|---|---|---|---|---|---|
| | | | | | Fold | | Improvement |
| | | | IIb- | IIaR- | wrt | IIb | wrt |
| Position | Mutations¹ | Variant # | Fold | Fold | Control | Selectivity | Control |
| | Strat1 control | 27293 | 415 | 35 | 1.00 | 12 | 1.0 |
| A_234 | A_L234A_strat1 | 26094 | 307 | 23 | 0.74 | 13 | 1.1 |
| | A_L234D_strat1 | 26105 | 636 | 29 | 1.53 | 22 | 1.9 |
| | A_L234E_strat1 | 26106 | 229 | 20 | 0.55 | 11 | 1.0 |
| | A_L234F_strat1 | 26098 | 547 | 28 | 1.32 | 19 | 1.6 |
| | A_L234G_strat1 | 26093 | 424 | 32 | 1.02 | 13 | 1.1 |
| | A_L234H_strat1 | 26109 | 372 | 23 | 0.90 | 16 | 1.4 |
| | A_L234I_strat1 | 26096 | 362 | 22 | 0.87 | 17 | 1.4 |
| | A_L234N_strat1 | 26104 | 460 | 27 | 1.11 | 17 | 1.5 |
| | A_L234P_strat1 | 26110 | 346 | 20 | 0.83 | 17 | 1.4 |
| | A_L234Q_strat1 | 26103 | 396 | 20 | 0.95 | 20 | 1.7 |
| | A_L234S_strat1 | 26102 | 372 | 22 | 0.90 | 17 | 1.4 |
| | A_L234T_strat1 | 26101 | 443 | 24 | 1.07 | 18 | 1.6 |
| | A_L234V_strat1 | 26095 | 335 | 21 | 0.81 | 16 | 1.4 |
| | A_L234W_strat1 | 26099 | 1,132 | 53 | 2.72 | 21 | 1.8 |
| | A_L234Y_strat1 | 26100 | 470 | 29 | 1.13 | 16 | 1.4 |
| A_235 | A_L235A_strat1 | 26112 | 285 | 15 | 0.68 | 19 | 1.6 |
| | A_L235D_strat1 | 26123 | 457 | 23 | 1.10 | 20 | 1.7 |
| | A_L235E_strat1 | 26124 | 466 | 25 | 1.12 | 19 | 1.6 |
| | A_L235F_strat1 | 26116 | 1,323 | 53 | 3.19 | 25 | 2.1 |
| | A_L235H_strat1 | 26127 | 270 | 15 | 0.65 | 18 | 1.5 |
| | A_L235I_strat1 | 26114 | 348 | 20 | 0.84 | 17 | 1.5 |
| | A_L235N_strat1 | 26122 | 229 | 13 | 0.55 | 17 | 1.4 |
| | A_L235P_strat1 | 26128 | 224 | 17 | 0.54 | 13 | 1.1 |
| | A_L235Q_strat1 | 26121 | 258 | 15 | 0.62 | 17 | 1.4 |
| | A_L235S_strat1 | 26120 | 245 | 19 | 0.59 | 13 | 1.1 |
| | A_L235T_strat1 | 26119 | 280 | 17 | 0.67 | 17 | 1.4 |
| | A_L235V_strat1 | 26113 | 333 | 19 | 0.80 | 18 | 1.5 |
| | A_L235W_strat1 | 26117 | 1,690 | 70 | 4.07 | 24 | 2.1 |
| | A_L235Y_strat1 | 26118 | 1,167 | 53 | 2.81 | 22 | 1.9 |
| A_236 | A_N236A_strat1 | 26130 | 358 | 27 | 0.86 | 13 | 1.1 |
| | A_N236D_strat1 | 26141 | 569 | 37 | 1.37 | 16 | 1.3 |
| | A_N236E_strat1 | 26142 | 405 | 30 | 0.98 | 13 | 1.1 |
| | A_N236F_strat1 | 26135 | 603 | 37 | 1.45 | 16 | 1.4 |
| | A_N236G_strat1 | 26129 | 315 | 35 | 0.76 | 9 | 0.8 |
| | A_N236H_strat1 | 26145 | 266 | 19 | 0.64 | 14 | 1.2 |
| | A_N236I_strat1 | 26133 | 585 | 37 | 1.41 | 16 | 1.3 |
| | A_N236L_strat1 | 26132 | 539 | 42 | 1.30 | 13 | 1.1 |
| | A_N236P_strat1 | 26146 | 235 | 16 | 0.57 | 14 | 1.2 |
| | A_N236Q_strat1 | 26140 | 373 | 26 | 0.90 | 15 | 1.2 |
| | A_N236S_strat1 | 26139 | 425 | 31 | 1.02 | 14 | 1.2 |
| | A_N236T_strat1 | 26138 | 387 | 25 | 0.93 | 16 | 1.3 |
| | A_N236V_strat1 | 26131 | 486 | 39 | 1.17 | 13 | 1.1 |
| | A_N236W_strat1 | 26136 | 779 | 45 | 1.88 | 17 | 1.5 |
| | A_N236Y_strat1 | 26137 | 883 | 55 | 2.13 | 16 | 1.4 |
| A_237 | A_A237F_strat1 | 26152 | 962 | 80 | 2.32 | 12 | 1.0 |
| | A_A237G_strat1 | 26147 | 3,395 | 194 | 8.17 | 17 | 1.5 |
| | A_A237H_strat1 | 26163 | 235 | 19 | 0.57 | 12 | 1.1 |
| | A_A237L_strat1 | 26149 | 252 | 12 | 0.61 | 20 | 1.7 |
| | A_A237N_strat1 | 26158 | 307 | 16 | 0.74 | 19 | 1.6 |
| | A_A237P_strat1 | 26164 | 389 | 45 | 0.94 | 9 | 0.7 |
| | A_A237S_strat1 | 26156 | 229 | 16 | 0.55 | 14 | 1.2 |
| | A_A237V_strat1 | 26148 | 245 | 31 | 0.59 | 8 | 0.7 |
| | A_A237W_strat1 | 26153 | 542 | 66 | 1.30 | 8 | 0.7 |
| | A_A237Y_strat1 | 26154 | 310 | 23 | 0.75 | 14 | 1.2 |
| A_239 | A_S239A_strat1 | 26166 | 725 | 35 | 1.75 | 21 | 1.8 |
| | A_S239D_strat1 | 26177 | 370 | 23 | 0.89 | 16 | 1.4 |
| | A_S239E_strat1 | 26178 | 383 | 42 | 0.92 | 9 | 0.8 |
| | A_S239F_strat1 | 26171 | 467 | 29 | 1.12 | 16 | 1.4 |
| | A_S239G_strat1 | 26165 | 639 | 29 | 1.54 | 22 | 1.9 |
| | A_S239H_strat1 | 26181 | 464 | 22 | 1.12 | 21 | 1.8 |
| | A_S239I_strat1 | 26169 | 502 | 31 | 1.21 | 16 | 1.4 |
| | A_S239L_strat1 | 26168 | 511 | 33 | 1.23 | 15 | 1.3 |
| | A_S239N_strat1 | 26176 | 590 | 36 | 1.42 | 16 | 1.4 |
| | A_S239Q_strat1 | 26175 | 497 | 29 | 1.20 | 17 | 1.5 |
| | A_S239R_strat1 | 26179 | 318 | 23 | 0.76 | 14 | 1.2 |
| | A_S239T_strat1 | 26174 | 765 | 37 | 1.84 | 21 | 1.8 |
| | A_S239V_strat1 | 26167 | 562 | 38 | 1.35 | 15 | 1.3 |
| | A_S239W_strat1 | 26172 | 269 | 33 | 0.65 | 8 | 0.7 |
| | A_S239Y_strat1 | 26173 | 575 | 26 | 1.38 | 22 | 1.9 |
| B_234 | B_L234A_strat1 | 26184 | 314 | 24 | 0.76 | 13 | 1.1 |
| | B_L234E_strat1 | 26197 | 241 | 20 | 0.58 | 12 | 1.0 |
| | B_L234F_strat1 | 26189 | 311 | 32 | 0.75 | 10 | 0.8 |
| | B_L234G_strat1 | 26183 | 418 | 35 | 1.01 | 12 | 1.0 |
| | B_L234H_strat1 | 26200 | 316 | 29 | 0.76 | 11 | 0.9 |
| | B_L234I_strat1 | 26187 | 274 | 25 | 0.66 | 11 | 0.9 |
| | B_L234K_strat1 | 26199 | 280 | 18 | 0.67 | 15 | 1.3 |
| | B_L234N_strat1 | 26195 | 323 | 28 | 0.78 | 12 | 1.0 |
| | B_L234P_strat1 | 26201 | 355 | 29 | 0.86 | 12 | 1.0 |
| | B_L234Q_strat1 | 26194 | 299 | 26 | 0.72 | 12 | 1.0 |
| | B_L234S_strat1 | 26193 | 270 | 27 | 0.65 | 10 | 0.9 |
| | B_L234T_strat1 | 26192 | 254 | 23 | 0.61 | 11 | 0.9 |
| | B_L234V_strat1 | 26185 | 240 | 24 | 0.58 | 10 | 0.8 |
| | B_L234W_strat1 | 26190 | 270 | 30 | 0.65 | 9 | 0.8 |
| | B_L234Y_strat1 | 26191 | 269 | 26 | 0.65 | 10 | 0.9 |
| B_235 | B_L235A_strat1 | 26203 | 236 | 19 | 0.57 | 12 | 1.0 |
| | B_L235D_strat1 | 26214 | 336 | 25 | 0.81 | 13 | 1.1 |
| | B_L235F_strat1 | 26207 | 312 | 33 | 0.75 | 9 | 0.8 |
| | B_L235G_strat1 | 26202 | 271 | 23 | 0.65 | 12 | 1.0 |
| | B_L235N_strat1 | 26213 | 383 | 30 | 0.92 | 13 | 1.1 |
| | B_L235S_strat1 | 26211 | 250 | 19 | 0.60 | 13 | 1.1 |
| | B_L235W_strat1 | 26208 | 360 | 32 | 0.87 | 11 | 1.0 |
| | B_L235Y_strat1 | 26209 | 287 | 30 | 0.69 | 9 | 0.8 |
| B_236 | B_D236E_strat1 | 26233 | 586 | 81 | 1.41 | 7 | 0.6 |
| | B_D236K_strat1 | 26235 | 346 | 17 | 0.83 | 20 | 1.7 |
| | B_D236N_strat1 | 26232 | 447 | 41 | 1.08 | 11 | 0.9 |
| | B_D236T_strat1 | 26229 | 715 | 92 | 1.72 | 8 | 0.7 |
| B_237 | B_F237I_strat1 | 26242 | 334 | 32 | 0.80 | 10 | 0.9 |
| | B_F237K_strat1 | 26253 | 382 | 41 | 0.92 | 9 | 0.8 |
| | B_F237L_strat1 | 26241 | 912 | 66 | 2.20 | 14 | 1.2 |
| | B_F237Q_strat1 | 26248 | 256 | 22 | 0.62 | 11 | 1.0 |
| | B_F237T_strat1 | 26246 | 271 | 25 | 0.65 | 11 | 0.9 |
| | B_F237V_strat1 | 26240 | 333 | 30 | 0.80 | 11 | 0.9 |
| | B_F237Y_strat1 | 26245 | 30 | 28 | 0.74 | 11 | 0.9 |
| B_239 | B_D239E_strat1 | 26269 | 269 | 30 | 0.65 | 9 | 0.8 |
| B_240 | B_V240I_strat1 | 26276 | 260 | 23 | 0.63 | 11 | 1.0 |
| | B_V240L_strat1 | 26275 | 297 | 22 | 0.72 | 13 | 1.1 |
| B_263 | B_V263T_strat1 | 26285 | 341 | 33 | 0.82 | 10 | 0.9 |
| B_264 | B_V264T_strat1 | 26291 | 295 | 28 | 0.71 | 11 | 0.9 |
| B_266 | B_V266I_strat1 | 26294 | 225 | 18 | 0.54 | 12 | 1.0 |
| B_267 | B_V267Q_strat1 | 26308 | 1,000 | 121 | 2.41 | 8 | 0.7 |
| B_268 | B_D268A_strat1 | 26315 | 401 | 36 | 0.97 | 11 | 0.9 |
| | B_D268E_strat1 | 26327 | 627 | 44 | 1.51 | 14 | 1.2 |
| | B_D268F_strat1 | 26320 | 568 | 47 | 1.37 | 12 | 1.0 |
| | B_D268I_strat1 | 26318 | 422 | 43 | 1.02 | 10 | 0.8 |
| | B_D268K_strat1 | 26329 | 278 | 37 | 0.67 | 8 | 0.6 |
| | B_D268L_strat1 | 26317 | 341 | 33 | 0.82 | 10 | 0.9 |
| | B_D268N_strat1 | 26326 | 239 | 25 | 0.57 | 10 | 0.8 |
| | B_D268P_strat1 | 26331 | 404 | 38 | 0.97 | 11 | 0.9 |
| | B_D268Q_strat1 | 26325 | 425 | 43 | 1.02 | 10 | 0.8 |
| | B_D268T_strat1 | 26323 | 239 | 21 | 0.57 | 11 | 0.9 |
| | B_D268V_strat1 | 26316 | 400 | 35 | 0.96 | 12 | 1.0 |
| | B_D268W_strat1 | 26321 | 341 | 37 | 0.82 | 9 | 0.8 |
| | B_D268Y_strat1 | 26322 | 506 | 51 | 1.22 | 10 | 0.8 |
| B_269 | B_E269Q_strat1 | 26343 | 253 | 17 | 0.61 | 15 | 1.2 |
| B_273 | B_V273A_strat1 | 26386 | 274 | 18 | 0.66 | 16 | 1.3 |
| | B_V273I_strat1 | 26388 | 436 | 27 | 1.05 | 16 | 1.3 |
| B_323 | B_V323A_strat1 | 26392 | 488 | 25 | 1.17 | 20 | 1.7 |
| | B_V323I_strat1 | 26394 | 269 | 16 | 0.65 | 17 | 1.4 |
| B_325* | B_S325*A_strat1 | 26399 | 487 | 27 | 1.17 | 18 | 1.5 |
| | B_S325*D_strat1 | 26410 | 436 | 33 | 1.05 | 13 | 1.1 |
| | B_S325*N_strat1 | 26409 | 400 | 25 | 0.96 | 16 | 1.4 |
| B_326* | B_T326*A_strat1 | 26417 | 337 | 19 | 0.81 | 18 | 1.5 |
| | B_T326*D_strat1 | 26428 | 1,120 | 55 | 2.70 | 21 | 1.7 |
| | B_T326*E_strat1 | 26429 | 817 | 39 | 1.97 | 21 | 1.8 |
| | B_T326*F_strat1 | 26422 | 465 | 26 | 1.12 | 18 | 1.5 |
| | B_T326*H_strat1 | 26432 | 508 | 27 | 1.22 | 19 | 1.6 |
| | B_T326*I_strat1 | 26420 | 468 | 26 | 1.13 | 18 | 1.5 |
| | B_T326*L_strat1 | 26419 | 382 | 19 | 0.92 | 20 | 1.7 |
| | B_T326*N_strat1 | 26427 | 757 | 41 | 1.82 | 19 | 1.6 |
| | B_T326*Q_strat1 | 26426 | 412 | 21 | 0.99 | 19 | 1.6 |
| | B_T326*S_strat1 | 26425 | 305 | 18 | 0.73 | 17 | 1.5 |
| | B_T326*V_strat1 | 26418 | 462 | 24 | 1.11 | 19 | 1.6 |
| | B_T326*W_strat1 | 26423 | 416 | 22 | 1.00 | 19 | 1.6 |
| | B_T326*Y_strat1 | 26424 | 373 | 21 | 0.90 | 18 | 1.5 |
| B_328* | B_F328*A_strat1 | 26453 | 353 | 23 | 0.85 | 15 | 1.3 |
| | B_F328*H_strat1 | 26468 | 290 | 15 | 0.70 | 20 | 1.7 |
| | B_F328*I_strat1 | 26456 | 244 | 17 | 0.59 | 14 | 1.2 |
| | B_F328*S_strat1 | 26461 | 250 | 13 | 0.60 | 19 | 1.6 |
| | B_F328*T_strat1 | 26460 | 261 | 18 | 0.63 | 15 | 1.2 |
| | B_F328*V_strat1 | 26454 | 452 | 33 | 1.09 | 14 | 1.2 |
| | B_F328*W_strat1 | 26458 | 345 | 20 | 0.83 | 18 | 1.5 |
| | B_F328*Y_strat1 | 26459 | 463 | 25 | 1.11 | 18 | 1.6 |
| B_329* | B_D329*E_strat1 | 26483 | 914 | 72 | 2.20 | 13 | 1.1 |
| | B_D329*G_strat1 | 26470 | 1,039 | 58 | 2.50 | 18 | 1.5 |
| | B_D329*I_strat1 | 26474 | 259 | 4 | 0.62 | 58 | 4.9 |
| | B_D329*L_strat1 | 26473 | 350 | 9 | 0.84 | 37 | 3.1 |
| | B_D329*P_strat1 | 26487 | 323 | 19 | 0.78 | 17 | 1.5 |
| | B_D329*Q_strat1 | 26481 | 348 | 23 | 0.84 | 15 | 1.3 |
| B_330* | B_G330*A_strat1 | 26488 | 2,829 | 128 | 6.81 | 22 | 1.9 |
| | B_G330*D_strat1 | 26500 | 282 | 10 | 0.68 | 29 | 2.5 |
| | B_G330*E_strat1 | 26501 | 857 | 45 | 2.06 | 19 | 1.6 |
| | B_G330*H_strat1 | 26504 | 1,571 | 61 | 3.78 | 26 | 2.2 |
| | B_G330*K_strat1 | 26503 | 1,025 | 27 | 2.47 | 39 | 3.3 |
| | B_G330*N_strat1 | 26499 | 1,334 | 108 | 3.21 | 12 | 1.0 |
| | B_G330*S_strat1 | 26497 | 2,825 | 193 | 6.80 | 15 | 1.2 |
| | B_G330*T_strat1 | 26496 | 2,166 | 149 | 5.21 | 15 | 1.2 |
| | B_G330*Y_strat1 | 26495 | 861 | 100 | 2.07 | 9 | 0.7 |
| B_331*A | B_Y331*AA_strat1 | 26525 | 209 | 14 | 0.50 | 15 | 1.3 |
| | B_Y331*AD_strat1 | 26535 | 346 | 22 | 0.83 | 16 | 1.4 |
| | B_Y331*AE_strat1 | 26536 | 219 | 15 | 0.53 | 15 | 1.2 |
| | B_Y331*AF_strat1 | 26530 | 489 | 28 | 1.18 | 18 | 1.5 |
| | B_Y331*AH_strat1 | 26539 | 216 | 15 | 0.52 | 15 | 1.2 |
| | B_Y331*AP_strat1 | 26540 | 325 | 22 | 0.78 | 15 | 1.3 |
| | B_Y331*AW_strat1 | 26531 | 411 | 21 | 0.99 | 20 | 1.7 |
| B_331*B | B_A331*BD_strat1 | 26553 | 219 | 18 | 0.53 | 12 | 1.0 |
| | B_A331*BE_strat1 | 26554 | 518 | 33 | 1.25 | 16 | 1.3 |
| | B_A331*BG_strat1 | 26541 | 329 | 22 | 0.79 | 15 | 1.2 |
| | B_A331*BH_strat1 | 26557 | 414 | 22 | 1.00 | 19 | 1.6 |
| | B_A331*BK_strat1 | 26556 | 632 | 33 | 1.52 | 19 | 1.6 |
| | B_A331*BL_strat1 | 26543 | 596 | 25 | 1.44 | 24 | 2.0 |
| | B_A331*BN_strat1 | 26552 | 318 | 21 | 0.77 | 15 | 1.3 |
| | B_A331*BQ_strat1 | 26551 | 516 | 30 | 1.24 | 17 | 1.5 |
| | B_A331*BR_strat1 | 26555 | 241 | 22 | 0.58 | 11 | 0.9 |
| B_332 | B_I332F_strat1 | 26563 | 408 | 23 | 0.98 | 18 | 1.5 |
| | B_I332L_strat1 | 26561 | 911 | 33 | 2.19 | 28 | 2.3 |

¹Mutation notation is in the format A_L234G_strat1, where “A” indicates the Fc chain, “L234G” indicates the mutation made with “L” representing the parental residue being replaced, 234 representing the position and G representing the replacement residue, and “strat1” specifies the parental CH2 mutations (A_G236N_G237A/B_G236D_G237F_S239D_S267V_H268D_Template1)

TABLE 6.26
Strategy 2 Variants Meeting Criteria B

| | | | | IIb- | | | IIb Selectivity |
|---|---|---|---|---|---|---|---|
| | | | | Fold | | | Improvement |
| | | | IIb- | wrt | IIaR- | IIb | wrt |
| Position | Mutations¹ | Variant # | Fold | Control | fold | Selectivity | Control |
| Control | Strat2 Control | 27294 | 92.9 | 1.0 | 7.0 | 13.4 | 1.0 |
| A_234 | A_F234A_strat2 | 26566 | 88.4 | 1.0 | 8.3 | 10.7 | 0.8 |
| | A_F234G_strat2 | 26565 | 55.2 | 0.6 | 4.1 | 13.6 | 1.0 |
| | A_F234H_strat2 | 26580 | 75.6 | 0.8 | 5.7 | 13.3 | 1.0 |
| | A_F234I_strat2 | 26569 | 132.0 | 1.4 | 14.7 | 9.0 | 0.7 |
| | A_F234L_strat2 | 26568 | 169.3 | 1.8 | 16.8 | 10.1 | 0.8 |
| | A_F234N_strat2 | 26575 | 91.7 | 1.0 | 6.3 | 14.6 | 1.1 |
| | A_F234P_strat2 | 26581 | 119.7 | 1.3 | 10.2 | 11.7 | 0.9 |
| | A_F234Q_strat2 | 26574 | 77.2 | 0.8 | 5.3 | 14.6 | 1.1 |
| | A_F234S_strat2 | 26573 | 89.5 | 1.0 | 6.2 | 14.5 | 1.1 |
| | A_F234T_strat2 | 26572 | 81.0 | 0.9 | 6.3 | 12.9 | 1.0 |
| | A_F234V_strat2 | 26567 | 145.7 | 1.6 | 14.5 | 10.0 | 0.8 |
| | A_F234W_strat2 | 26570 | 145.8 | 1.6 | 11.9 | 12.2 | 0.9 |
| | A_F234Y_strat2 | 26571 | 62.4 | 0.7 | 5.3 | 11.7 | 0.9 |
| A_235 | A_L235A_strat2 | 26583 | 54.2 | 0.6 | 3.8 | 14.3 | 1.1 |
| | A_L235D_strat2 | 26593 | 65.2 | 0.7 | 2.5 | 26.3 | 2.0 |
| | A_L235E_strat2 | 26594 | 52.9 | 0.6 | 3.9 | 13.6 | 1.0 |
| | A_L235F_strat2 | 26586 | 97.3 | 1.0 | 6.7 | 14.4 | 1.1 |
| | A_L235H_strat2 | 26597 | 78.4 | 0.8 | 5.2 | 15.0 | 1.1 |
| | A_L235I_strat2 | 26585 | 69.0 | 0.7 | 5.2 | 13.2 | 1.0 |
| | A_L235P_strat2 | 26598 | 56.3 | 0.6 | 4.2 | 13.4 | 1.0 |
| | A_L235Q_strat2 | 26591 | 47.5 | 0.5 | 4.0 | 12.0 | 0.9 |
| | A_L235S_strat2 | 26590 | 47.6 | 0.5 | 3.3 | 14.4 | 1.1 |
| | A_L235T_strat2 | 26589 | 47.0 | 0.5 | 3.1 | 15.3 | 1.1 |
| | A_L235V_strat2 | 26584 | 59.6 | 0.6 | 4.2 | 14.4 | 1.1 |
| | A_L235W_strat2 | 26587 | 90.4 | 1.0 | 6.8 | 13.3 | 1.0 |
| | A_L235Y_strat2 | 26588 | 116.9 | 1.3 | 7.1 | 16.4 | 1.2 |
| A_236 | A_N236D_strat2 | 26610 | 62.7 | 0.7 | 5.6 | 11.1 | 0.8 |
| | A_N236F_strat2 | 26604 | 171.2 | 1.8 | 11.6 | 14.7 | 1.1 |
| | A_N236G_strat2 | 26599 | 198.4 | 2.1 | 14.8 | 13.4 | 1.0 |
| | A_N236Q_strat2 | 26609 | 47.8 | 0.5 | 7.1 | 6.7 | 0.5 |
| | A_N236W_strat2 | 26605 | 227.1 | 2.4 | 15.2 | 15.0 | 1.1 |
| | A_N236Y_strat2 | 26606 | 195.1 | 2.1 | 11.7 | 16.7 | 1.2 |
| A_237 | A_G237A_strat2 | 26616 | 58.6 | 0.6 | 4.7 | 12.6 | 0.9 |
| | A_G237F_strat2 | 26620 | 442.1 | 4.8 | 39.9 | 11.1 | 0.8 |
| | A_G237L_strat2 | 26618 | 115.6 | 1.2 | 6.1 | 19.1 | 1.4 |
| | A_G237N_strat2 | 26626 | 48.3 | 0.5 | 3.3 | 14.5 | 1.1 |
| | A_G237T_strat2 | 26623 | 47.9 | 0.5 | 4.3 | 11.1 | 0.8 |
| | A_G237W_strat2 | 26621 | 193.3 | 2.1 | 17.7 | 10.9 | 0.8 |
| | A_G237Y_strat2 | 26622 | 162.2 | 1.7 | 10.6 | 15.2 | 1.1 |
| A_239 | A_S239A_strat2 | 26634 | 62.6 | 0.7 | 4.6 | 13.6 | 1.0 |
| | A_S239D_strat2 | 26644 | 78.0 | 0.8 | 5.2 | 14.9 | 1.1 |
| | A_S239E_strat2 | 26645 | 70.5 | 0.8 | 5.0 | 14.1 | 1.1 |
| | A_S239G_strat2 | 26633 | 81.7 | 0.9 | 4.3 | 18.9 | 1.4 |
| | A_S239I_strat2 | 26637 | 51.7 | 0.6 | 5.3 | 9.8 | 0.7 |
| | A_S239L_strat2 | 26636 | 86.0 | 0.9 | 5.3 | 16.4 | 1.2 |
| | A_S239N_strat2 | 26643 | 69.2 | 0.7 | 4.3 | 16.1 | 1.2 |
| | A_S239Q_strat2 | 26642 | 62.9 | 0.7 | 4.4 | 14.3 | 1.1 |
| | A_S239R_strat2 | 26646 | 56.1 | 0.6 | 6.4 | 8.7 | 0.7 |
| | A_S239V_strat2 | 26635 | 74.7 | 0.8 | 5.3 | 14.2 | 1.1 |
| A_264 | A_V264A_strat2 | 26650 | 48.4 | 0.5 | 4.5 | 10.7 | 0.8 |
| | A_V264F_strat2 | 26654 | 94.3 | 1.0 | 6.2 | 15.1 | 1.1 |
| | A_V264I_strat2 | 26652 | 83.2 | 0.9 | 6.8 | 12.1 | 0.9 |
| | A_V264L_strat2 | 26651 | 70.3 | 0.8 | 5.7 | 12.4 | 0.9 |
| | A_V264T_strat2 | 26655 | 71.2 | 0.8 | 7.0 | 10.1 | 0.8 |
| A_266 | A_V266I_strat2 | 26658 | 88.8 | 1.0 | 6.0 | 14.9 | 1.1 |
| A_267 | A_S267A_strat2 | 26663 | 62.1 | 0.7 | 2.6 | 23.6 | 1.8 |
| | A_S267G_strat2 | 26662 | 49.1 | 0.5 | 3.3 | 14.7 | 1.1 |
| | A_S267H_strat2 | 26675 | 51.2 | 0.6 | 3.0 | 17.3 | 1.3 |
| | A_S267I_strat2 | 26666 | 68.0 | 0.7 | 5.4 | 12.6 | 0.9 |
| | A_S267N_strat2 | 26672 | 51.6 | 0.6 | 3.8 | 13.7 | 1.0 |
| | A_S267P_strat2 | 26676 | 58.3 | 0.6 | 3.9 | 15.0 | 1.1 |
| | A_S267T_strat2 | 26670 | 71.2 | 0.8 | 6.0 | 11.9 | 0.9 |
| | A_S267V_strat2 | 26664 | 52.5 | 0.6 | 4.3 | 12.2 | 0.9 |
| A_268 | A_Q268A_strat2 | 26678 | 64.4 | 0.7 | 4.8 | 13.3 | 1.0 |
| | A_Q268D_strat2 | 26688 | 71.6 | 0.8 | 5.3 | 13.5 | 1.0 |
| | A_Q268E_strat2 | 26689 | 55.1 | 0.6 | 4.2 | 13.0 | 1.0 |
| | A_Q268F_strat2 | 26682 | 86.0 | 0.9 | 8.0 | 10.8 | 0.8 |
| | A_Q268G_strat2 | 26677 | 69.9 | 0.8 | 4.4 | 16.0 | 1.2 |
| | A_Q268H_strat2 | 26692 | 94.0 | 1.0 | 6.7 | 14.0 | 1.0 |
| | A_Q268I_strat2 | 26681 | 60.8 | 0.7 | 4.3 | 14.1 | 1.1 |
| | A_Q268K_strat2 | 26691 | 84.5 | 0.9 | 5.6 | 15.1 | 1.1 |
| | A_Q268L_strat2 | 26680 | 64.8 | 0.7 | 5.0 | 13.0 | 1.0 |
| | A_Q268N_strat2 | 26687 | 71.5 | 0.8 | 5.7 | 12.5 | 0.9 |
| | A_Q268P_strat2 | 26693 | 96.3 | 1.0 | 6.0 | 16.1 | 1.2 |
| | A_Q268R_strat2 | 26690 | 79.4 | 0.9 | 5.3 | 14.9 | 1.1 |
| | A_Q268S_strat2 | 26686 | 65.7 | 0.7 | 4.9 | 13.3 | 1.0 |
| | A_Q268T_strat2 | 26685 | 94.1 | 1.0 | 5.9 | 15.9 | 1.2 |
| | A_Q268V_strat2 | 26679 | 66.6 | 0.7 | 6.6 | 10.1 | 0.8 |
| | A_Q268W_strat2 | 26683 | 92.2 | 1.0 | 6.3 | 14.6 | 1.1 |
| | A_Q268Y_strat2 | 26684 | 89.6 | 1.0 | 6.6 | 13.7 | 1.0 |
| A_269 | A_E269A_strat2 | 26695 | 59.8 | 0.6 | 4.5 | 13.2 | 1.0 |
| | A_E269D_strat2 | 26706 | 73.6 | 0.8 | 5.0 | 14.7 | 1.1 |
| | A_E269F_strat2 | 26699 | 65.7 | 0.7 | 6.0 | 11.0 | 0.8 |
| | A_E269G_strat2 | 26694 | 48.3 | 0.5 | 4.0 | 12.1 | 0.9 |
| | A_E269H_strat2 | 26709 | 64.5 | 0.7 | 4.5 | 14.4 | 1.1 |
| | A_E269I_strat2 | 26698 | 71.8 | 0.8 | 5.1 | 14.1 | 1.1 |
| | A_E269K_strat2 | 26708 | 65.8 | 0.7 | 4.4 | 15.0 | 1.1 |
| | A_E269L_strat2 | 26697 | 74.0 | 0.8 | 5.1 | 14.5 | 1.1 |
| | A_E269N_strat2 | 26705 | 54.7 | 0.6 | 3.7 | 14.8 | 1.1 |
| | A_E269P_strat2 | 26710 | 52.8 | 0.6 | 3.9 | 13.4 | 1.0 |
| | A_E269Q_strat2 | 26704 | 67.2 | 0.7 | 4.7 | 14.3 | 1.1 |
| | A_E269R_strat2 | 26707 | 63.2 | 0.7 | 4.0 | 15.8 | 1.2 |
| | A_E269S_strat2 | 26703 | 60.4 | 0.6 | 5.1 | 11.8 | 0.9 |
| | A_E269T_strat2 | 26702 | 56.0 | 0.6 | 4.8 | 11.6 | 0.9 |
| | A_E269V_strat2 | 26696 | 66.8 | 0.7 | 5.0 | 13.3 | 1.0 |
| | A_E269W_strat2 | 26700 | 83.1 | 0.9 | 4.6 | 17.9 | 1.3 |
| | A_E269Y_strat2 | 26701 | 67.2 | 0.7 | 4.6 | 14.7 | 1.1 |
| A_270 | A_D270A_strat2 | 26712 | 49.9 | 0.5 | 3.8 | 13.0 | 1.0 |
| | A_D270E_strat2 | 26723 | 109.0 | 1.2 | 7.3 | 15.0 | 1.1 |
| | A_D270F_strat2 | 26716 | 86.7 | 0.9 | 6.4 | 13.5 | 1.0 |
| | A_D270H_strat2 | 26726 | 61.7 | 0.7 | 4.1 | 14.9 | 1.1 |
| | A_D270I_strat2 | 26715 | 57.9 | 0.6 | 3.8 | 15.2 | 1.1 |
| | A_D270N_strat2 | 26722 | 67.4 | 0.7 | 4.0 | 16.7 | 1.3 |
| | A_D270Q_strat2 | 26721 | 51.7 | 0.6 | 3.6 | 14.6 | 1.1 |
| | A_D270S_strat2 | 26720 | 49.5 | 0.5 | 3.9 | 12.7 | 0.9 |
| | A_D270T_strat2 | 26719 | 64.2 | 0.7 | 4.9 | 13.2 | 1.0 |
| | A_D270W_strat2 | 26717 | 53.5 | 0.6 | 3.7 | 14.5 | 1.1 |
| | A_D270Y_strat2 | 26718 | 54.6 | 0.6 | 4.5 | 12.1 | 0.9 |
| A_271 | A_P271D_strat2 | 26740 | 50.5 | 0.5 | 3.4 | 14.9 | 1.1 |
| | A_P271E_strat2 | 26741 | 59.3 | 0.6 | 5.1 | 11.7 | 0.9 |
| | A_P271G_strat2 | 26728 | 65.4 | 0.7 | 4.8 | 13.5 | 1.0 |
| | A_P271H_strat2 | 26744 | 59.3 | 0.6 | 3.9 | 15.1 | 1.1 |
| | A_P271I_strat2 | 26732 | 56.9 | 0.6 | 4.4 | 12.9 | 1.0 |
| | A_P271K_strat2 | 26743 | 55.7 | 0.6 | 4.3 | 12.9 | 1.0 |
| | A_P271L_strat2 | 26731 | 66.6 | 0.7 | 4.5 | 14.7 | 1.1 |
| | A_P271N_strat2 | 26739 | 50.9 | 0.5 | 3.4 | 14.9 | 1.1 |
| | A_P271Q_strat2 | 26738 | 56.1 | 0.6 | 4.0 | 14.1 | 1.1 |
| | A_P271R_strat2 | 26742 | 47.1 | 0.5 | 4.4 | 10.7 | 0.8 |
| | A_P271V_strat2 | 26730 | 54.4 | 0.6 | 3.6 | 15.0 | 1.1 |
| | A_P271W_strat2 | 26734 | 52.5 | 0.6 | 4.3 | 12.3 | 0.9 |
| A_272 | A_E272A_strat2 | 26746 | 70.8 | 0.8 | 4.5 | 15.9 | 1.2 |
| | A_E272D_strat2 | 26757 | 66.6 | 0.7 | 4.9 | 13.5 | 1.0 |
| | A_E272F_strat2 | 26750 | 61.2 | 0.7 | 4.5 | 13.5 | 1.0 |
| | A_E272G_strat2 | 26745 | 51.6 | 0.6 | 4.5 | 11.6 | 0.9 |
| | A_E272H_strat2 | 26760 | 74.1 | 0.8 | 4.9 | 15.3 | 1.1 |
| | A_E272I_strat2 | 26749 | 83.8 | 0.9 | 6.1 | 13.7 | 1.0 |
| | A_E272L_strat2 | 26748 | 81.3 | 0.9 | 5.3 | 15.2 | 1.1 |
| | A_E272N_strat2 | 26756 | 57.6 | 0.6 | 3.6 | 15.8 | 1.2 |
| | A_E272S_strat2 | 26754 | 61.0 | 0.7 | 4.1 | 14.7 | 1.1 |
| | A_E272T_strat2 | 26753 | 62.8 | 0.7 | 4.3 | 14.7 | 1.1 |
| | A_E272V_strat2 | 26747 | 89.7 | 1.0 | 6.0 | 14.9 | 1.1 |
| | A_E272W_strat2 | 26751 | 60.1 | 0.6 | 5.4 | 11.2 | 0.8 |
| | A_E272Y_strat2 | 26752 | 79.8 | 0.9 | 4.6 | 17.5 | 1.3 |
| A_273 | A_V273A_strat2 | 26762 | 52.7 | 0.6 | 3.2 | 16.5 | 1.2 |
| A_323 | A_V323A_strat2 | 26768 | 47.5 | 0.5 | 3.2 | 14.7 | 1.1 |
| | A_V323I_strat2 | 26770 | 81.2 | 0.9 | 5.9 | 13.8 | 1.0 |
| | A_V323L_strat2 | 26769 | 83.5 | 0.9 | 6.4 | 13.1 | 1.0 |
| A_326 | A_K326A_strat2 | 26792 | 66.3 | 0.7 | 4.7 | 14.1 | 1.1 |
| | A_K326D_strat2 | 26803 | 65.4 | 0.7 | 4.8 | 13.7 | 1.0 |
| | A_K326H_strat2 | 26806 | 48.0 | 0.5 | 3.5 | 13.8 | 1.0 |
| | A_K326N_strat2 | 26802 | 88.7 | 1.0 | 6.3 | 14.2 | 1.1 |
| | A_K326Q_strat2 | 26801 | 55.8 | 0.6 | 4.1 | 13.5 | 1.0 |
| | A_K326R_strat2 | 26805 | 69.5 | 0.7 | 5.1 | 13.7 | 1.0 |
| | A_K326S_strat2 | 26800 | 82.6 | 0.9 | 5.9 | 14.1 | 1.1 |
| | A_K326T_strat2 | 26799 | 59.5 | 0.6 | 4.4 | 13.6 | 1.0 |
| A_327 | A_G327A_strat2 | 26808 | 60.7 | 0.7 | 4.4 | 13.8 | 1.0 |
| | A_G327E_strat2 | 26818 | 58.5 | 0.6 | 3.8 | 15.4 | 1.2 |
| A_330 | A_K330A_strat2 | 26840 | 58.0 | 0.6 | 3.8 | 15.3 | 1.1 |
| | A_K330H_strat2 | 26854 | 50.0 | 0.5 | 4.1 | 12.2 | 0.9 |
| | A_K330Q_strat2 | 26849 | 53.5 | 0.6 | 3.1 | 17.2 | 1.3 |
| | A_K330R_strat2 | 26853 | 62.6 | 0.7 | 4.3 | 14.6 | 1.1 |
| | A_K330S_strat2 | 26848 | 51.6 | 0.6 | 3.7 | 14.0 | 1.0 |
| | A_K330T_strat2 | 26847 | 53.3 | 0.6 | 2.4 | 22.6 | 1.7 |
| A_331 | A_S331A_strat2 | 26857 | 88.7 | 1.0 | 6.4 | 13.8 | 1.0 |
| | A_S331D_strat2 | 26867 | 61.5 | 0.7 | 4.7 | 13.0 | 1.0 |
| | A_S331E_strat2 | 26868 | 63.7 | 0.7 | 5.2 | 12.2 | 0.9 |
| | A_S331H_strat2 | 26871 | 54.7 | 0.6 | 3.6 | 15.0 | 1.1 |
| | A_S331P_strat2 | 26872 | 113.8 | 1.2 | 8.9 | 12.8 | 1.0 |
| | A_S331Q_strat2 | 26865 | 60.5 | 0.7 | 4.5 | 13.6 | 1.0 |
| A_332 | A_I332A_strat2 | 26873 | 92.0 | 1.0 | 6.7 | 13.8 | 1.0 |
| | A_I332L_strat2 | 26875 | 52.6 | 0.6 | 3.9 | 13.3 | 1.0 |
| | A_I332T_strat2 | 26878 | 82.6 | 0.9 | 6.7 | 12.3 | 0.9 |
| | A_I332V_strat2 | 26874 | 65.4 | 0.7 | 4.5 | 14.4 | 1.1 |
| B_234 | B_L234A_strat2 | 26880 | 60.7 | 0.7 | 4.5 | 13.3 | 1.0 |
| | B_L234D_strat2 | 26890 | 54.1 | 0.6 | 4.5 | 11.9 | 0.9 |
| | B_L234E_strat2 | 26891 | 49.5 | 0.5 | 3.5 | 14.0 | 1.0 |
| | B_L234F_strat2 | 26883 | 67.8 | 0.7 | 5.4 | 12.6 | 0.9 |
| | B_L234G_strat2 | 26879 | 62.3 | 0.7 | 6.3 | 9.8 | 0.7 |
| | B_L234I_strat2 | 26882 | 72.6 | 0.8 | 4.8 | 15.1 | 1.1 |
| | B_L234N_strat2 | 26889 | 60.5 | 0.7 | 5.6 | 10.7 | 0.8 |
| | B_L234P_strat2 | 26895 | 79.8 | 0.9 | 5.4 | 14.8 | 1.1 |
| | B_L234Q_strat2 | 26888 | 51.4 | 0.6 | 4.0 | 12.9 | 1.0 |
| | B_L234S_strat2 | 26887 | 58.5 | 0.6 | 5.2 | 11.3 | 0.8 |
| | B_L234T_strat2 | 26886 | 50.2 | 0.5 | 4.1 | 12.3 | 0.9 |
| | B_L234V_strat2 | 26881 | 56.2 | 0.6 | 4.2 | 13.5 | 1.0 |
| | B_L234W_strat2 | 26884 | 57.1 | 0.6 | 4.3 | 13.3 | 1.0 |
| | B_L234Y_strat2 | 26885 | 56.5 | 0.6 | 4.8 | 11.7 | 0.9 |
| B_235 | B_L235A_strat2 | 26897 | 58.3 | 0.6 | 4.9 | 12.0 | 0.9 |
| | B_L235D_strat2 | 26907 | 81.6 | 0.9 | 4.6 | 17.7 | 1.3 |
| | B_L235F_strat2 | 26900 | 121.4 | 1.3 | 9.6 | 12.7 | 1.0 |
| | B_L235G_strat2 | 26896 | 55.4 | 0.6 | 5.1 | 10.8 | 0.8 |
| | B_L235H_strat2 | 26911 | 53.3 | 0.6 | 4.8 | 11.1 | 0.8 |
| | B_L235N_strat2 | 26906 | 100.4 | 1.1 | 7.9 | 12.7 | 0.9 |
| | B_L235W_strat2 | 26901 | 77.8 | 0.8 | 7.5 | 10.3 | 0.8 |
| | B_L235Y_strat2 | 26902 | 117.5 | 1.3 | 10.0 | 11.7 | 0.9 |
| B_236 | B_D236E_strat2 | 26924 | 63.7 | 0.7 | 8.6 | 7.4 | 0.6 |
| B_237 | B_G237A_strat2 | 26929 | 223.7 | 2.4 | 12.0 | 18.7 | 1.4 |
| | B_G237D_strat2 | 26940 | 304.2 | 3.3 | 13.1 | 23.3 | 1.7 |
| | B_G237E_strat2 | 26941 | 143.1 | 1.5 | 9.0 | 15.9 | 1.2 |
| | B_G237F_strat2 | 26933 | 148.4 | 1.6 | 8.7 | 17.0 | 1.3 |
| | B_G237H_strat2 | 26944 | 73.4 | 0.8 | 5.4 | 13.5 | 1.0 |
| | B_G237I_strat2 | 26932 | 87.1 | 0.9 | 4.8 | 18.0 | 1.3 |
| | B_G237K_strat2 | 26943 | 115.6 | 1.2 | 8.1 | 14.4 | 1.1 |
| | B_G237L_strat2 | 26931 | 296.1 | 3.2 | 14.2 | 20.9 | 1.6 |
| | B_G237N_strat2 | 26939 | 541.7 | 5.8 | 27.5 | 19.7 | 1.5 |
| | B_G237Q_strat2 | 26938 | 358.6 | 3.9 | 22.4 | 16.0 | 1.2 |
| | B_G237R_strat2 | 26942 | 51.4 | 0.6 | 3.5 | 14.7 | 1.1 |
| | B_G237S_strat2 | 26937 | 141.4 | 1.5 | 11.8 | 12.0 | 0.9 |
| | B_G237T_strat2 | 26936 | 91.5 | 1.0 | 5.5 | 16.6 | 1.2 |
| | B_G237V_strat2 | 26930 | 75.3 | 0.8 | 5.1 | 14.7 | 1.1 |
| | B_G237W_strat2 | 26934 | 224.1 | 2.4 | 12.4 | 18.1 | 1.4 |
| | B_G237Y_strat2 | 26935 | 220.7 | 2.4 | 12.7 | 17.4 | 1.3 |
| B_239 | B_D239L_strat2 | 26949 | 54.1 | 0.6 | 5.0 | 10.7 | 0.8 |
| B_240 | B_V240I_strat2 | 26965 | 58.0 | 0.6 | 3.9 | 14.9 | 1.1 |
| | B_V240L_strat2 | 26964 | 57.6 | 0.6 | 4.2 | 13.8 | 1.0 |
| | B_V240T_strat2 | 26968 | 48.4 | 0.5 | 5.2 | 9.3 | 0.7 |
| B_264 | B_V264L_strat2 | 26976 | 104.4 | 1.1 | 5.8 | 18.0 | 1.3 |
| | B_V264T_strat2 | 26980 | 121.2 | 1.3 | 13.7 | 8.9 | 0.7 |
| B_266 | B_L266I_strat2 | 26983 | 58.7 | 0.6 | 7.1 | 8.3 | 0.6 |
| | B_L266V_strat2 | 26981 | 48.2 | 0.5 | 4.0 | 12.1 | 0.9 |
| B_267 | B_A267Q_strat2 | 26996 | 105.1 | 1.1 | 10.0 | 10.5 | 0.8 |
| B_268 | B_D268A_strat2 | 27010 | 61.5 | 0.7 | 7.2 | 8.5 | 0.6 |
| | B_D268E_strat2 | 27033 | 81.3 | 0.9 | 8.3 | 9.8 | 0.7 |
| | B_D268F_strat2 | 27019 | 56.5 | 0.6 | 6.7 | 8.4 | 0.6 |
| | B_D268N_strat2 | 27031 | 48.3 | 0.5 | 5.7 | 8.5 | 0.6 |
| | B_D268Q_strat2 | 27029 | 73.9 | 0.8 | 8.3 | 8.9 | 0.7 |
| | B_D268S_strat2 | 27027 | 52.0 | 0.6 | 6.4 | 8.1 | 0.6 |
| | B_D268V_strat2 | 27012 | 60.9 | 0.7 | 6.6 | 9.2 | 0.7 |
| | B_D268W_strat2 | 27021 | 55.1 | 0.6 | 6.7 | 8.3 | 0.6 |
| | B_D268Y_strat2 | 27023 | 60.5 | 0.7 | 6.9 | 8.8 | 0.7 |
| B_269 | B_E269D_strat2 | 27068 | 73.1 | 0.8 | 7.4 | 9.9 | 0.7 |
| | B_E269T_strat2 | 27060 | 58.0 | 0.6 | 5.5 | 10.5 | 0.8 |
| | B_E269V_strat2 | 27047 | 57.5 | 0.6 | 4.6 | 12.6 | 0.9 |
| B_271 | B_P271G_strat2 | 27112 | 104.0 | 1.1 | 13.7 | 7.6 | 0.6 |
| B_272 | B_E272A_strat2 | 27151 | 80.5 | 0.9 | 6.1 | 13.1 | 1.0 |
| | B_E272D_strat2 | 27174 | 59.4 | 0.6 | 6.3 | 9.5 | 0.7 |
| | B_E272I_strat2 | 27157 | 55.7 | 0.6 | 5.7 | 9.7 | 0.7 |
| | B_E272K_strat2 | 27178 | 63.5 | 0.7 | 6.2 | 10.3 | 0.8 |
| | B_E272L_strat2 | 27155 | 66.0 | 0.7 | 6.5 | 10.2 | 0.8 |
| | B_E272P_strat2 | 27182 | 105.6 | 1.1 | 11.4 | 9.3 | 0.7 |
| | B_E272Q_strat2 | 27170 | 54.5 | 0.6 | 4.1 | 13.2 | 1.0 |
| | B_E272R_strat2 | 27176 | 61.2 | 0.7 | 5.5 | 11.2 | 0.8 |
| | B_E272T_strat2 | 27166 | 46.9 | 0.5 | 5.2 | 9.0 | 0.7 |
| | B_E272V_strat2 | 27152 | 47.6 | 0.5 | 4.9 | 9.8 | 0.7 |
| B_273 | B_V273A_strat2 | 27184 | 50.4 | 0.5 | 5.3 | 9.6 | 0.7 |
| | B_V273I_strat2 | 27189 | 100.2 | 1.1 | 9.5 | 10.5 | 0.8 |
| | B_V273L_strat2 | 27186 | 72.2 | 0.8 | 5.1 | 14.3 | 1.1 |
| | B_V273T_strat2 | 27195 | 166.4 | 1.8 | 16.6 | 10.0 | 0.8 |

¹Mutation notation is in the format A_F234G_strat2, where “A” indicates the Fc chain, “F234G” indicates the mutation made with “F” representing the parental residue being replaced, 234 representing the position and G representing the replacement residue, and “strat2” specifies the parental CH2 mutations (A_L234F_G236N_H268Q_A327G_A330K_P331S/B_G236D_S239D_V266L_S267A_H268D)

TABLE 6.27
Strategy 3 Variants Meeting Criteria B

| | | | | | IIb- | | IIb Selectivity |
|---|---|---|---|---|---|---|---|
| | | | | | Fold | | Improvement |
| Loop | | | IIb- | IIaR- | wrt | IIb | wrt |
| Template | Mutations¹ | Variant # | Fold | Fold | Control | Selectivity | Control |
| Control | Strat 3 control | 27362 | 9 | 1 | | 9 | |
| Template | template1_S325*A_A331*BN_strat3 | 27374 | 332.1 | 21.2 | 35.2 | 15.7 | 1.8 |
| 1 | template1_T326*H_F328*H_D329*G_W327*T_strat3 | 27396 | 11.1 | 2.4 | 1.2 | 4.6 | 0.5 |
| | template1_T326*H_F328*T_D329*G_W327*T_strat3 | 27398 | 14.9 | 2.1 | 1.6 | 6.9 | 0.8 |
| | template1_T326*H_W327*D_F328*T_D329*G_strat3 | 27395 | 16.4 | 2.7 | 1.7 | 6.1 | 0.7 |
| | template1_T326*H_W327*W_F328*D_D329*D_strat3 | 27372 | 92.2 | 6.4 | 9.8 | 14.4 | 1.6 |
| | template1_T326*H_W327*W_F328*E_D329*D_A331*BN_{—} | 27382 | 76.9 | 7.7 | 8.2 | 10.0 | 1.1 |
| | strat3 | | | | | | |
| | template1_T326*H_W327*W_F328*E_D329*D_S325*A_{—} | 27388 | 87.3 | 9.1 | 9.3 | 9.6 | 1.1 |
| | A331*BN_strat3 | | | | | | |
| | template1_T326*H_W327*W_F328*E_D329*D_strat3 | 27364 | 114.4 | 8.8 | 12.1 | 13.0 | 1.5 |
| | template1_T326*H_W327*W_F328*E_D329*G_A331*BN_{—} | 27383 | 608.5 | 28.1 | 64.5 | 21.7 | 2.5 |
| | strat3 | | | | | | |
| | template1_T326*H_W327*W_F328*E_D329*G_S325*A_{—} | 27389 | 505.1 | 28.9 | 53.6 | 17.5 | 2.0 |
| | A331*BN_strat3 | | | | | | |
| | template1_T326*H_W327*W_F328*E_D329*G_S325*A_{—} | 27377 | 327.6 | 30.8 | 34.8 | 10.6 | 1.2 |
| | strat3 | | | | | | |
| | template1_T326*H_W327*W_F328*E_D329*G_strat3 | 27365 | 838.3 | 44.8 | 88.9 | 18.7 | 2.1 |
| | template1_T326*H_W327*W_F328*F_D329*D_A331*BN_{—} | 27385 | 337.3 | 20.9 | 35.8 | 16.1 | 1.8 |
| | strat3 | | | | | | |
| | template1_T326*H_W327*W_F328*F_D329*D_S325*A_{—} | 27391 | 402.4 | 26.8 | 42.7 | 15.0 | 1.7 |
| | A331*BN_strat3 | | | | | | |
| | template1_T326*H_W327*W_F328*F_D329*D_S325*A_{—} | 27379 | 486.3 | 32.0 | 51.6 | 15.2 | 1.7 |
| | strat3 | | | | | | |
| | template1_T326*H_W327*W_F328*H_D329*D_strat3 | 27373 | 332.1 | 17.4 | 35.2 | 19.1 | 2.2 |
| | template1_T326*H_W327*W_F328*H_D329*G_strat3 | 27393 | 393.0 | 21.3 | 41.7 | 18.4 | 2.1 |
| | template1_T326*H_W327*W_F328*N_D329*D_strat3 | 27367 | 148.7 | 8.7 | 15.8 | 17.2 | 2.0 |
| | template1_T326*H_W327*W_F328*Q_D329*D_strat3 | 27368 | 187.9 | 13.2 | 19.9 | 14.3 | 1.6 |
| | template1_T326*H_W327*W_F328*Q_D329*G_A331*BN_{—} | 27384 | 255.9 | 15.5 | 27.1 | 16.5 | 1.9 |
| | strat3 | | | | | | |
| | template1_T326*H_W327*W_F328*Q_D329*G_S325*A_{—} | 27390 | 247.9 | 16.0 | 26.3 | 15.5 | 1.8 |
| | A331*BN_strat3 | | | | | | |
| | template1_T326*H_W327*W_F328*Q_D329*G_S325*A_{—} | 27378 | 322.7 | 19.7 | 34.2 | 16.4 | 1.9 |
| | strat3 | | | | | | |
| | template1_T326*H_W327*W_F328*Q_D329*G_strat3 | 27366 | 450.1 | 22.5 | 47.7 | 20.0 | 2.3 |
| | template1_T326*H_W327*W_F328*S_D329*D_A331*BN_{—} | 27381 | 172.3 | 12.2 | 18.3 | 14.2 | 1.6 |
| | strat3 | | | | | | |
| | template1_T326*H_W327*W_F328*S_D329*D_S325*A_{—} | 27387 | 108.4 | 8.1 | 11.5 | 13.4 | 1.5 |
| | A331*BN_strat3 | | | | | | |
| | template1_T326*H_W327*W_F328*S_D329*D_S325*A_{—} | 27375 | 164.8 | 10.2 | 17.5 | 16.2 | 1.9 |
| | strat3 | | | | | | |
| | template1_T326*H_W327*W_F328*S_D329*D_strat3 | 27363 | 188.4 | 10.9 | 20.0 | 17.2 | 2.0 |
| | template1_T326*H_W327*W_F328*T_D329*D_strat3 | 27371 | 237.0 | 16.7 | 25.1 | 14.2 | 1.6 |
| | template1_T326*T_F328*H_D329*G_W327*T_strat3 | 27397 | 15.8 | 2.7 | 1.7 | 5.9 | 0.7 |
| | template1_T326*T_W327*W_F328*H_D329*G_strat3 | 27394 | 429.6 | 22.6 | 45.6 | 19.0 | 2.2 |
| | template1_T326*T_W327*W_F328*Q_D329*G_strat3 | 27369 | 386.3 | 18.7 | 41.0 | 20.6 | 2.4 |
| | template1_T326*T_W327*W_F328*S_D329*D_A331*BN_{—} | 27386 | 138.2 | 9.3 | 14.7 | 14.8 | 1.7 |
| | strat3 | | | | | | |
| | template1_T326*T_W327*W_F328*S_D329*D_S325*A_{—} | 27392 | 74.5 | 5.5 | 7.9 | 13.5 | 1.5 |
| | A331*BN_strat3 | | | | | | |
| | template1_T326*T_W327*W_F328*S_D329*G_strat3 | 27370 | 296.5 | 19.4 | 31.5 | 15.3 | 1.8 |
| Template | template7_A331*BV_G325*F_strat3 | 27448 | 62.1 | 4.8 | 6.6 | 12.9 | 1.5 |
| 7 | template7_A331*BV_strat3 | 27445 | 40.3 | 5.3 | 4.3 | 7.5 | 0.9 |
| | template7_A331*BY_strat3 | 27446 | 82.5 | 6.6 | 8.8 | 12.5 | 1.4 |
| | template7_E328*E_E329*N_A331*BV_G325*F_strat3 | 27461 | 63.5 | 4.4 | 6.7 | 14.4 | 1.6 |
| | template7_E328*E_E329*N_A331*BV_strat3 | 27449 | 42.2 | 4.5 | 4.5 | 9.4 | 1.1 |
| | template7_E328*E_E329*N_A331*BY_strat3 | 27453 | 107.9 | 7.1 | 11.4 | 15.3 | 1.7 |
| | template7_E328*E_E329*N_G325*F_strat3 | 27457 | 52.0 | 5.6 | 5.5 | 9.4 | 1.1 |
| | template7_E328*E_E329*N_strat3 | 27440 | 40.8 | 4.1 | 4.3 | 9.9 | 1.1 |
| | template7_E328*H_E329*R_A331*BV_G325*F_strat3 | 27463 | 34.9 | 2.6 | 3.7 | 13.5 | 1.5 |
| | template7_E328*H_E329*R_A331*BV_strat3 | 27451 | 12.4 | 1.2 | 1.3 | 10.3 | 1.2 |
| | template7_E328*H_E329*R_A331*BY_strat3 | 27455 | 86.7 | 2.8 | 9.2 | 30.5 | 3.5 |
| | template7_E328*H_E329*R_G325*F_strat3 | 27459 | 42.1 | 3.7 | 4.5 | 11.3 | 1.3 |
| | template7_E328*H_E329*R_strat3 | 27442 | 7.5 | 0.7 | 0.8 | 10.7 | 1.2 |
| | template7_E328*H_E329*T_strat3 | 27444 | 22.0 | 2.0 | 2.3 | 11.1 | 1.3 |
| | template7_E328*Q_E329*S_A331*BV_G325*F_strat3 | 27464 | 56.4 | 4.3 | 6.0 | 13.2 | 1.5 |
| | template7_E328*Q_E329*S_A331*BV_strat3 | 27452 | 48.6 | 5.2 | 5.2 | 9.3 | 1.1 |
| | template7_E328*Q_E329*S_A331*BY_strat3 | 27456 | 59.7 | 4.0 | 6.3 | 15.0 | 1.7 |
| | template7_E328*Q_E329*S_G325*F_strat3 | 27460 | 54.8 | 5.1 | 5.8 | 10.7 | 1.2 |
| | template7_E328*Q_E329*S_strat3 | 27443 | 34.0 | 3.2 | 3.6 | 10.7 | 1.2 |
| | template7_E328*T_E329*N_A331*BV_G325*F_strat3 | 27462 | 55.8 | 4.2 | 5.9 | 13.1 | 1.5 |
| | template7_E328*T_E329*N_A331*BV_strat3 | 27450 | 49.7 | 6.4 | 5.3 | 7.7 | 0.9 |
| | template7_E328*T_E329*N_A331*BY_strat3 | 27454 | 55.6 | 4.0 | 5.9 | 13.9 | 1.6 |
| | template7_E328*T_E329*N_G325*F_strat3 | 27458 | 50.2 | 5.0 | 5.3 | 10.1 | 1.2 |
| | template7_E328*T_E329*N_strat3 | 27441 | 49.0 | 4.2 | 5.2 | 11.7 | 1.3 |
| | template7_G325*F_strat3 | 27447 | 62.5 | 6.4 | 6.6 | 9.8 | 1.1 |
| Template | template7_A331*BV_G325*F_strat3-HF | 27487 | 71.9 | 8.6 | 7.6 | 8.4 | 1.0 |
| 7-HF2 | template7_A331*BV_strat3-HF | 27485 | 39.6 | 7.0 | 4.2 | 5.7 | 0.6 |
| | template7_E328*E_E329*N_A331*BV_strat3-HF | 27488 | 32.4 | 5.6 | 3.4 | 5.8 | 0.7 |
| | template7_E328*E_E329*N_strat3-HF | 27484 | 35.4 | 6.7 | 3.8 | 5.3 | 0.6 |
| | template7_G325*F_strat3-HF | 27486 | 103.7 | 13.7 | 11.0 | 7.6 | 0.9 |
| Template | template19_V325*A_strat3 | 27465 | 77.1 | 14.9 | 8.2 | 5.2 | 0.6 |
| 19 | | | | | | | |
| Template | template66_D325*A_strat3 | 27414 | 27.9 | 2.5 | 3.0 | 11.2 | 1.3 |
| 66 | template66_D327*D_Q328*D_N329*E_Q330*D_D325*A_{—} | 27428 | 41.1 | 5.0 | 4.4 | 8.3 | 0.9 |
| | strat3 | | | | | | |
| | template66_D327*D_Q328*D_N329*E_Q330*D_I332Q_{—} | 27434 | 48.2 | 6.6 | 5.1 | 7.3 | 0.8 |
| | D325*A_strat3 | | | | | | |
| | template66_D327*D_Q328*D_N329*E_Q330*D_I332W_{—} | 27422 | 20.6 | 2.0 | 2.2 | 10.3 | 1.2 |
| | strat3 | | | | | | |
| | template66_D327*D_Q328*D_N329*E_Q330*D_strat3 | 27399 | 54.8 | 5.8 | 5.8 | 9.5 | 1.1 |
| | template66_D327*D_Q328*D_N329*S_Q330*Q_strat3 | 27412 | 45.1 | 3.6 | 4.8 | 12.6 | 1.4 |
| | template66_D327*D_Q328*E_N329*D_Q330*D_D325*A_{—} | 27430 | 35.4 | 4.5 | 3.8 | 7.8 | 0.9 |
| | strat3 | | | | | | |
| | template66_D327*D_Q328*E_N329*D_Q330*D_I332Q_{—} | 27436 | 47.0 | 6.6 | 5.0 | 7.1 | 0.8 |
| | D325*A_strat3 | | | | | | |
| | template66_D327*D_Q328*E_N329*D_Q330*D_I332W_{—} | 27424 | 17.1 | 1.8 | 1.8 | 9.6 | 1.1 |
| | strat3 | | | | | | |
| | template66_D327*D_Q328*E_N329*D_Q330*D_strat3 | 27401 | 58.2 | 4.2 | 6.2 | 13.9 | 1.6 |
| | template66_D327*D_Q328*E_N329*E_Q330*Q_D325*A_{—} | 27431 | 33.2 | 3.9 | 3.5 | 8.5 | 1.0 |
| | strat3 | | | | | | |
| | template66_D327*D_Q328*E_N329*E_Q330*Q_I332Q_{—} | 27437 | 50.7 | 7.2 | 5.4 | 7.0 | 0.8 |
| | D325*A_strat3 | | | | | | |
| | template66_D327*D_Q328*E_N329*E_Q330*Q_I332Q_strat3 | 27419 | 35.8 | 4.2 | 3.8 | 8.5 | 1.0 |
| | template66_D327*D_Q328*E_N329*E_Q330*Q_I332W_{—} | 27425 | 12.8 | 1.4 | 1.4 | 9.4 | 1.1 |
| | strat3 | | | | | | |
| | template66_D327*D_Q328*E_N329*E_Q330*Q_strat3 | 27402 | 44.5 | 4.0 | 4.7 | 11.1 | 1.3 |
| | template66_D327*D_Q328*H_N329*D_Q330*Q_D325*A_{—} | 27432 | 21.7 | 2.3 | 2.3 | 9.3 | 1.1 |
| | strat3 | | | | | | |
| | template66_D327*D_Q328*H_N329*D_Q330*Q_I332Q_{—} | 27438 | 30.7 | 3.7 | 3.3 | 8.3 | 1.0 |
| | D325*A_strat3 | | | | | | |
| | template66_D327*D_Q328*H_N329*D_Q330*Q_I332Q_{—} | 27420 | 35.1 | 3.8 | 3.7 | 9.1 | 1.0 |
| | strat3 | | | | | | |
| | template66_D327*D_Q328*H_N329*D_Q330*Q_I332W_{—} | 27426 | 18.7 | 1.8 | 2.0 | 10.3 | 1.2 |
| | strat3 | | | | | | |
| | template66_D327*D_Q328*H_N329*D_Q330*Q_strat3 | 27403 | 47.8 | 3.0 | 5.1 | 16.1 | 1.8 |
| | template66_D327*D_Q328*N_N329*D_Q330*D_strat3 | 27405 | 75.3 | 4.4 | 8.0 | 17.2 | 2.0 |
| | template66_D327*D_Q328*P_N329*D_Q330*Q_D325*A_{—} | 27429 | 45.6 | 4.9 | 4.8 | 9.2 | 1.1 |
| | strat3 | | | | | | |
| | template66_D327*D_Q328*P_N329*D_Q330*Q_I332Q_{—} | 27435 | 43.5 | 6.2 | 4.6 | 7.0 | 0.8 |
| | D325*A_strat3 | | | | | | |
| | template66_D327*D_Q328*P_N329*D_Q330*Q_I332W_{—} | 27423 | 14.8 | 1.5 | 1.6 | 9.7 | 1.1 |
| | strat3 | | | | | | |
| | template66_D327*D_Q328*P_N329*D_Q330*Q_strat3 | 27400 | 56.4 | 4.8 | 6.0 | 11.9 | 1.4 |
| | template66_D327*D_Q328*S_N329*T_Q330*D_D325*A_{—} | 27433 | 33.4 | 3.4 | 3.5 | 9.9 | 1.1 |
| | strat3 | | | | | | |
| | template66_D327*D_Q328*S_N329*T_Q330*D_I332Q_{—} | 27439 | 34.5 | 4.9 | 3.7 | 7.0 | 0.8 |
| | D325*A_strat3 | | | | | | |
| | template66_D327*D_Q328*S_N329*T_Q330*D_I332Q_strat3 | 27421 | 49.5 | 5.3 | 5.2 | 9.4 | 1.1 |
| | template66_D327*D_Q328*S_N329*T_Q330*D_I332W_{—} | 27427 | 18.9 | 2.1 | 2.0 | 8.8 | 1.0 |
| | strat3 | | | | | | |
| | template66_D327*D_Q328*S_N329*T_Q330*D_strat3 | 27404 | 57.6 | 3.3 | 6.1 | 17.7 | 2.0 |
| | template66_D327*D_Q328*S_N329*T_Q330*Q_strat3 | 27408 | 52.5 | 3.2 | 5.6 | 16.6 | 1.9 |
| | template66_D327*D_Q328*T_N329*D_Q330*D_strat3 | 27406 | 64.9 | 3.7 | 6.9 | 17.5 | 2.0 |
| | template66_D327*D_Q328*T_N329*S_Q330*Q_strat3 | 27410 | 48.8 | 2.9 | 5.2 | 16.6 | 1.9 |
| | template66_D327*N_Q328*D_N329*E_Q330*Q_strat3 | 27407 | 32.3 | 3.1 | 3.4 | 10.3 | 1.2 |
| | template66_D327*N_Q328*H_N329*N_Q330*D_strat3 | 27411 | 26.5 | 1.9 | 2.8 | 13.6 | 1.6 |
| | template66_I332Q_D325*A_strat3 | 27415 | 34.8 | 4.7 | 3.7 | 7.5 | 0.9 |
| | template66_I332Q_strat3 | 27409 | 37.5 | 4.3 | 4.0 | 8.6 | 1.0 |
| | template66_I332W_strat3 | 27413 | 16.0 | 1.9 | 1.7 | 8.3 | 1.0 |
| Template | template66_D325*A_strat3-HF | 27478 | 24.4 | 3.2 | 2.6 | 7.7 | 0.9 |
| 66-HF3 | template66_D327*D_Q328*D_N329*E_Q330*D_strat3-HF | 27475 | 34.5 | 5.9 | 3.7 | 5.8 | 0.7 |
| | template66_D327*D_Q328*D_N329*E_Q330*D_I332Q_{—} | 27482 | 54.0 | 9.0 | 5.7 | 6.0 | 0.7 |
| | D325*A_strat3-HF | | | | | | |
| | template66_D327*D_Q328*P_N329*D_Q330*Q_I332Q_{—} | 27483 | 45.1 | 7.9 | 4.8 | 5.7 | 0.7 |
| | D325*A_strat3-HF | | | | | | |
| | template66_D327*D_Q328*P_N329*D_Q330*Q_I332Q_strat3 | 27481 | 29.3 | 4.5 | 3.1 | 6.5 | 0.7 |
| | -HF | | | | | | |
| | template66_D327*D_Q328*P_N329*D_Q330*Q_strat3-HF | 27476 | 28.6 | 4.7 | 3.0 | 6.1 | 0.7 |
| | template66_I332Q_D325*A_strat3-HF | 27479 | 33.6 | 6.2 | 3.6 | 5.5 | 0.6 |
| | template66_I332Q_strat3-HF | 27477 | 28.6 | 3.8 | 3.0 | 7.5 | 0.9 |
| Template | template151_E328*E_E329*D_R331*S_strat3 | 27473 | 78.8 | 9.1 | 8.4 | 8.7 | 1.0 |
| 151 | template151_E328*E_E329*D_strat3 | 27470 | 52.9 | 5.3 | 5.6 | 9.9 | 1.1 |
| | template151_E328*E_E329*D_Y331*BI_R331*S_strat3 | 27474 | 163.5 | 10.0 | 17.3 | 16.3 | 1.9 |
| | template151_E328*E_E329*D_Y331*BI_strat3 | 27472 | 144.1 | 9.5 | 15.3 | 15.2 | 1.7 |
| | template151_E328*H_E329*N_strat3 | 27469 | 18.9 | 1.5 | 2.0 | 12.2 | 1.4 |
| | template151_E328*H_E329*N_Y331*BI_strat3 | 27471 | 43.9 | 2.3 | 4.7 | 19.4 | 2.2 |
| | template151_R331*S_strat3 | 27467 | 80.3 | 8.7 | 8.5 | 9.3 | 1.1 |
| | template151_Y331*BI_strat3 | 27466 | 128.8 | 8.6 | 13.7 | 15.0 | 1.7 |
| | template151_Y331*BQ_strat3 | 27468 | 118.9 | 14.2 | 12.6 | 8.4 | 1.0 |

¹Mutation notation is in the format “template1_T326*H_strat3,” where “template1” indicates the parental loop template, “T326*H” indicates the mutation made with “T” representing the parental residue being replaced, 326* representing the position and H representing the replacement residue, and “strat3” specifies the parental CH2 mutations (A_G236N_G237A/B_G236D_G237F_S239D_S267V_H268D).
²Template 7-HF indicates that the starting loop template was a modified version of Template 7 having the following sequence: GTDEEGKGAT [SEQ ID NO: 143]
³Template 66-HF indicates that the starting loop template was a modified version of Template 66 having the following sequence: DTDQNQGEVT [SEQ ID NO: 161]

1.-2. (canceled)

3. A heterodimeric Fc variant comprising a first Fc polypeptide and a second Fc polypeptide,
one of the Fc polypeptides comprising a replacement of amino acids 325 to 331 with a polypeptide between 8 and 15 amino acids in length,
wherein the heterodimeric Fc variant has increased selectivity of binding to FcγRIIb as compared to a parental Fc region,
wherein the heterodimeric Fc variant is a variant of an immunoglobulin G (IgG) Fc,
and wherein the numbering of amino acids is according to the EU index.

4. The heterodimeric Fc variant according to claim 3, wherein the polypeptide comprises:
(a) an amino acid sequence as set forth in any one of SEQ ID NOs: 4, 5, 6, 7, 8, 9, 10, 11, 12, 13 or 14, or
(b) an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations.

5. The heterodimeric Fc variant according to claim 3, wherein the polypeptide comprises an amino acid sequence of Formula (I), Formula (Ia), Formula (Ib), Formula (II), Formula (III), Formula (IV), Formula (V) or Formula (VI):
Formula (I):
X¹X²WX³X⁴X⁵GX⁶X⁷T (I)
wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, F, G, H, K, L, N, Q or R.
Formula (Ia):
X¹X²WX³X⁴X⁵GYX⁶T (Ia)
wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y, and
X⁶ is A, D, E, F, G, H, K, L, N, Q or R;
Formula (Ib):
X¹X²WX³X⁴GGYX⁵T (Ib)
wherein:
X¹ is A or S;
X² is A, D, E, F, H, I, L, N, Q, T, V or W;
X³ is D, E, F, H, N, Q, S, T or Y;
X⁴ is D, G, I or L, and
X⁵ is A, F, H, K, L or N;
Formula (II):
X¹LDX²X³GKGX⁴V (II)
wherein:
X¹ is F or G;
X² is E, H, Q or T;
X³ is E, N, R, S or T, and
X⁴ is A, Y or V;
Formula (III):
X¹TDEX²GKGX³T (III)
wherein:
X¹ is F or G;
X² is E or N, and
X³ is A or V;
Formula (IV):
X¹FX²X³X⁴X⁵GEVV (IV)
wherein:
X¹ is A or D;
X² is D or N;
X³ is D, E, H, N, P, Q, S or T;
X⁴ is D, E, N, S or T, and
X⁵ is D or Q;
Formula (V):
X¹TDX²X³X⁴GEVT (V)
wherein:
X¹ is A or D;
X² is D, P or Q;
X³ is D, E or N, and
X⁴ is D or Q;
Formula (VI):
LTDX¹X²GX³PX⁴R (VI)
wherein:
X¹ is E or H;
X² is D, E or N;
X³ is R or S, and
X⁴ is I, Q or Y.

6. The heterodimeric Fc variant according to claim 5, wherein the polypeptide comprises:
(a) an amino acid sequence as set forth in any one of SEQ ID NOs: 4-172, or
(b) an amino acid sequence as set forth in any one of SEQ ID NOs: 4-90, or
(c) an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90, or
(d) an amino acid sequence as set forth in any one of SEQ ID NOs: 6, 8, 47, 68 or 73.

7. The heterodimeric Fc variant according to claim 3, further comprising one or more additional amino acid mutations in the CH2 domain of the heterodimeric Fc variant.

8. The heterodimeric Fc variant according to claim 7, wherein the one or more additional amino acid mutations comprise a mutation at position 236.

9. (canceled)

10. The heterodimeric Fc variant according to claim 8, wherein the one or more additional amino acid mutations comprise a symmetrical mutation at position 236 in the first and second Fc polypeptides, and wherein the mutation at position 236 is selected from G236D, G236N and G236K.

11. (canceled)

12. The heterodimeric Fc variant according to claim 8, wherein the one or more additional amino acid mutations comprise an asymmetrical mutation at position 236 wherein the replacement of amino acids 325 to 331 is in the second Fc polypeptide, and wherein:
(a) the first Fc polypeptide comprises a mutation at position 236 selected from G236A, G236D, G236E, G236F, G236H, G236I, G236L, G236N, G236P, G236Q, G236S, G236T, G236V, G236W and G236Y, and the second Fc polypeptide comprises a mutation at position 236 selected from G236D, G236E, G236K, G236N and G236T, or
(b) the first Fc polypeptide comprises a mutation at position 236 selected from G236A, G236D, G236E, G236F, G236H, G236I, G236L, G236N, G236P, G236Q, G236S, G236T, G236V, G236W and G236Y, and the second Fc polypeptide comprises the mutation G236D or does not comprise a mutation at position 236.

13. The heterodimeric Fc variant according to claim 8, wherein the replacement of amino acids 325 to 331 is in the second Fc polypeptide, and the second Fc polypeptide further comprises one or mutations selected from S239D, S239E, V266I, V266L, S267A, S267I, S267V, S267Q and H268D.

14. (canceled)

15. The heterodimeric Fc variant according to claim 8, wherein the replacement of amino acids 325 to 331 is in the second Fc polypeptide, wherein the first Fc polypeptide further comprises a mutation at one or more of positions 234, 235, 237 and 239, and wherein:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

16. (canceled)

17. The heterodimeric Fc variant according to claim 8, wherein the replacement of amino acids 325 to 331 is in the second Fc polypeptide, wherein the second Fc polypeptide further comprises a mutation at one or more of positions 234, 235, 237, 240, 263, 264, 266, 269, 271, 273, 323 and 332, and wherein:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

18. A method of preparing a heterodimeric Fc variant having increased selectivity for a target receptor as compared to a parental Fc region, the heterodimeric Fc variant comprising a first Fc polypeptide and a second Fc polypeptide, the method comprising:
(a) using an in silico model of the parental Fc region complexed with the target receptor:
(i) inserting a sequence of one or more amino acid residues into a natural loop of one of the Fc polypeptides such that the natural loop is extended in length to provide a candidate variant,
(ii) determining the distance of at least one of the amino acid residues of the inserted sequence from a target amino acid residue in the receptor, and
(iii) selecting the candidate variant as the heterodimeric Fc variant if the at least one amino acid residue of the inserted sequence is within a heavy atom to heavy atom distance of 3 Å of the target amino acid residue in the receptor,
(b) preparing nucleic acid encoding the heterodimeric Fc variant,
(c) expressing the nucleic acid in a host cell to provide the heterodimeric Fc variant,
wherein the target receptor is FcγRIIb.

19. A heterodimeric Fc variant comprising a first Fc polypeptide and a second Fc polypeptide, the heterodimeric Fc variant having increased selectivity of binding to FcγRIIb as compared to a parental Fc region, the heterodimeric Fc variant comprising an asymmetric mutation at position 236,
wherein one of the Fc polypeptides comprises the mutation G236N or G236D,
wherein the heterodimeric Fc variant is a variant of an immunoglobulin G (IgG) Fc,
and wherein the numbering of amino acids is according to the EU index.

20. The heterodimeric Fc variant according to claim 19, wherein:
(a) the first Fc polypeptide comprises the mutation G236N or G236D, and the second Fc polypeptide does not comprise a mutation at position 236, or
(b) the first Fc polypeptide comprises the mutation G236N or G236D, and the second Fc polypeptide comprises a different mutation at position 236, or
(c) the first Fc polypeptide comprises the mutation G236N, and the second Fc polypeptide comprises the mutation G236D, G236K or G236S, or
(d) the first Fc polypeptide comprises the mutation G236N, and the second Fc polypeptide comprises the mutation G236D, or
(e) the first Fc polypeptide comprises the mutation G236D, and the second Fc polypeptide comprises the mutation G236N, G236Q, G236K, G236E or G236H.

21. The heterodimeric Fc variant according to claim 19, wherein the first Fc polypeptide and/or the second Fc polypeptide further comprises one or more additional amino acid mutations in the CH2 domain of the heterodimeric Fc variant.

22. The heterodimeric Fc variant according to claim 21, wherein the second Fc polypeptide further comprises one or mutations selected from S239D, S239E, V266I, V266L, S267A, S267I, S267V, S267Q and H268D.

23. The heterodimeric Fc variant according to claim 21, wherein the second Fc polypeptide further comprises:
a) the mutation S239D or S239E; or
b) the mutation H268D, or
c) the mutation S239D or S239E, and the mutation H268D.

24. (canceled)

25. The heterodimeric Fc variant according to claim 19, wherein the second Fc polypeptide further comprises the mutation S267A, S267I or S267V.

26. The heterodimeric Fc variant according to claim 19, wherein amino acids 325 to 331 in the second Fc polypeptide are replaced with a polypeptide between 8 and 15 amino acids in length.

27. The heterodimeric Fc variant according to claim 26, wherein the polypeptide comprises:
(a) an amino acid sequence as set forth in any one of SEQ ID NOs: 4, 5, 6, 7, 8, 9, 10, 11, 12, 13 or 14, or
(b) an amino acid sequence that is a variant of the sequence as set forth in any one of SEQ ID NOs: 4, 5, 6, 7, 8, 9, 10, 11, 12, 13 or 14, wherein the variant comprises 1, 2, 3, 4 or 5 amino acid mutations.

28. The heterodimeric Fc variant according to claim 26, wherein the polypeptide comprises an amino acid sequence of Formula (I), Formula (Ia), Formula (Ib), Formula (II), Formula (III), Formula (IV), Formula (V) or Formula (VI):
Formula (I):
X¹X²WX³X⁴X⁵GX⁶X⁷T (I)
wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y;
X⁶ is A, D, E, F, H, P, W or Y, and
X⁷ is A, D, E, F, G, H, K, L, N, Q or R.
Formula (Ia):
X¹X²WX³X⁴X⁵GYX⁶T (Ia)
wherein:
X¹ is A, D, N or S;
X² is A, D, E, F, H, I, L, N, Q, S, T, V, W or Y;
X³ is A, D, E, F, H, I, N, Q, S, T, V, W or Y;
X⁴ is D, E, G, I, L, P or Q;
X⁵ is A, D, E, G, H, K, N, R, S, T or Y, and
X⁶ is A, D, E, F, G, H, K, L, N, Q or R;
Formula (Ib):
X¹X²WX³X⁴GGYX⁵T (Ib)
wherein:
X¹ is A or S;
X² is A, D, E, F, H, I, L, N, Q, T, V or W;
X³ is D, E, F, H, N, Q, S, T or Y;
X⁴ is D, G, I or L, and
X⁵ is A, F, H, K, L or N;
Formula (II):
Formula (II):
X¹LDX²X³GKGX⁴V (II)
wherein:
X¹ is F or G;
X² is E, H, Q or T;
X³ is E, N, R, S or T, and
X⁴ is A, Y or V;
Formula (III):
X¹TDEX²GKGX³T (III)
wherein:
X¹ is F or G;
X² is E or N, and
X³ is A or V;
Formula (IV):
Formula (IV):
X¹FX²X³X⁴X⁵GEVV (IV)
wherein:
X¹ is A or D;
X² is D or N;
X³ is D, E, H, N, P, Q, S or T;
X⁴ is D, E, N, S or T, and
X⁵ is D or Q;
Formula (V):
X¹TDX²X³X⁴GEVT (V)
wherein:
X¹ is A or D;
X² is D, P or Q;
X³ is D, E or N, and
X⁴ is D or Q;
Formula (VI):
LTDX¹X²GX³PX⁴R (VI)
wherein:
X¹ is E or H;
X² is D, E or N;
X³ is R or S, and
X⁴ is I, Q or Y.

29. The heterodimeric Fc variant according to claim 26, wherein the polypeptide comprises:
(a) an amino acid sequence as set forth in any one of SEQ ID NOs: 4-172, or
(b) an amino acid sequence as set forth in any one of SEQ ID NOs: 4-90, or
(c) an amino acid sequence as set forth in any one of SEQ ID NOs: 6, 8, 9, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90, or
(d) an amino acid sequence as set forth in any one of SEQ ID NOs: 6, 8, 47, 68 or 73.

30. The heterodimeric Fc variant according to claim 19, wherein the second Fc polypeptide further comprises the mutation S267V.

31. (canceled)

32. The heterodimeric Fc variant according to claim 19, wherein the first Fc polypeptide and/or the second Fc polypeptide further comprises a mutation at position 237, and wherein:
(a) the first Fc polypeptide or the second Fc polypeptide comprises the mutation G236N and the same Fc polypeptide further comprises a mutation selected from G237A, G237D, G237F, G237H, G237L, G237N, G237P, G237S, G237V, G237W and G237Y, or
(b) the first Fc polypeptide or the second Fc polypeptide comprises the mutation G236D and the same Fc polypeptide further comprises a mutation selected from G237F, G237I, G237K, G237L, G237Q, G237T, G237V and G237Y.

33. (canceled)

34. The heterodimeric Fc variant according to claim 19, wherein the first Fc polypeptide comprises the mutation G236N, wherein the first Fc polypeptide further comprises a mutation at one or more of positions 234, 235, 237 and 239, and wherein:
(i) the mutation at position 234 is selected from L234A, L234D, L234E, L234F, L234G, L234H, L234I, L234N, L234P, L234Q, L234S, L234T, L234V, L234W and L234Y,
(ii) the mutation at position 235 is selected from L235A, L235D, L235E, L235F, L235H, L235I, L235N, L235P, L235Q, L235S, L235T, L235V, L235W and L235Y,
(iii) the mutation at position 237 is selected from G237A, G237D, G237F, G237H, G237L, G237N, G237P, G237S, G237V, G237W and G237Y, and
(iv) the mutation at position 239 is selected from S239A, S239D, S239E, S239F, S239G, S239H, S239I, S239L, S239N, S239Q, S239R, S239T, S239V, S239W and S239Y.

35. (canceled)

36. The heterodimeric Fc variant according to claim 19, wherein the second Fc polypeptide comprises the mutation G236D, wherein the second Fc polypeptide further comprises a mutation at one or more of positions 234, 235, 237, 240, 263, 264, 266, 269, 271, 273, 323 and 332, and wherein:
(i) the mutation at position 234 is selected from L234A, L234E, L234F, L234G, L234H, L234I, L234K, L234N, L234P, L234Q, L234S, L234T, L234V, L234W and L234Y,
(ii) the mutation at position 235 is selected from L235A, L235D, L235F, L235G, L235N, L235S, L235W and L235Y,
(iii) the mutation at position 237 is selected from G237F, G237I, G237K, G237L, G237Q, G237T, G237V and G237Y,
(iv) the mutation at position 240 is selected from V240I and V240L,
(v) the mutation at position 263 is V263T,
(vi) the mutation at position 264 is V264T,
(vii) the mutation at position 266 is V266I,
(viii) the mutation at position 269 is E269Q,
(ix) the mutation at position 271 is P271D,
(x) the mutation at position 273 is selected from V273A and V273I,
(xi) the mutation at position 323 is selected from V323A and V323I, and
(xii) the mutation at position 332 is selected from I332F and I332L.

37. The heterodimeric Fc variant according to claim 3, wherein the heterodimeric Fc variant comprises the amino acid mutations as set out for any one of the variants shown in Table 6.22, 6.24, 6.25 or 6.27.

38. The heterodimeric Fc variant according to claim 3, wherein:
(i) the first Fc polypeptide comprises the mutations G236N_G237D, and the second Fc polypeptide comprises the mutations Template 1 (D329*I)+G236D_G237F_S239D_S267V_H268D (Variant 31186);
(ii) the first Fc polypeptide comprises the mutations L235F_G236N_G237A, and the second Fc polypeptide comprises the mutations Template 1 (D329*I)+G236D_G237F_S239D_S267V_H268D (Variant 31187);
(iii) the first Fc polypeptide comprises the mutations L235F_G236N_G237A, and the second Fc polypeptide comprises the mutations Template 1 (G330*K)+G236D_G237F_S239D_S267V_H268D (Variant 31188);
(iv) the first Fc polypeptide comprises the mutations G236N_G237D, and the second Fc polypeptide comprises the mutations Template 7 (E328*H_E329*R_A331*BY)+G236D_G237F_S239D_S267V_H268D (Variant 31191);
(v) the first Fc polypeptide comprises the mutations L235F_G236N_G237A, and the second Fc polypeptide comprises the mutations Template 1 (D329*I)+G236D_G237F_S239D_S267V_H268D_I332L (Variant 31213);
(vi) the first Fc polypeptide comprises the mutations L235F_G236N_G237A_T250V_A287F, and the second Fc polypeptide comprises the mutations Template 1 (D329*I)+G236D_G237F_S239D_T250V_S267V_H268D_A287F (Variant 31274);
(vii) the first Fc polypeptide comprises the mutations L235F_G236N_G237A_T250V_M428F, and the second Fc polypeptide comprises the mutations Template 1 (D329*I)+G236D_G237F_S239D_T250V_S267V_H268D_M428F (Variant 31275);
(viii) the first Fc polypeptide comprises the mutations L235F_G236N_G237A_A287F_M428F, and the second Fc polypeptide comprises the mutations Template 1 (D329*I)+G236D_G237F_S239D_S267V_H268D_A287F_M428F (Variant 31276);
(ix) the first Fc polypeptide comprises the mutations G236N_G237D, and the second Fc polypeptide comprises the mutations Template 1 (D329*I)+G236D_G237F_S239D_S267V_H268D_I332L (Variant 32210);
(x) the first Fc polypeptide comprises the mutations G236N_G237E, and the second Fc polypeptide comprises the mutations Template 1 (D329*I)+G236D_G237F_S239D_S267V_H268D_I332L (Variant 32211);
(xi) the first Fc polypeptide comprises the mutation G236N, and the second Fc polypeptide comprises the mutations Template 1 (D329*I)+G236D_G237F_S239D_S267V_H268D_I332L (Variant 32212);
(xii) the first Fc polypeptide comprises the mutations L235D_G236N_G237A, and the second Fc polypeptide comprises the mutations Template 1 (D329*I)+G236D_G237F_S239D_S267V_H268D_I332L (Variant 32226);
(xiii) the first Fc polypeptide comprises the mutations L235E_G236N_G237A, and the second Fc polypeptide comprises the mutations Template 1 (D329*I)+G236D_G237F_S239D_S267V_H268D_I332L (Variant 32227);
(xiv) the first Fc polypeptide comprises the mutations L235V_G236N_G237A, and the second Fc polypeptide comprises the mutations Template 1 (D329*I)+G236D_G237F_S239D_S267V_H268D_I332L (Variant 32230);
(xv) the first Fc polypeptide comprises the mutations L235Y_G236N_G237A, and the second Fc polypeptide comprises the mutations Template 1 (D329*I)+G236D_G237F_S239D_S267V_H268D_I332L (Variant 32231);
(xvi) the first Fc polypeptide comprises the mutations G236N_G237A_S239P, and the second Fc polypeptide comprises the mutations Template 1 (D329*I)+G236D_G237F_S239D_S267V_H268D_I332L (Variant 32242);
(xvii) the first Fc polypeptide comprises the mutations L234D_G236N_G237A, and the second Fc polypeptide comprises the mutations Template 7+G236D_G237F_S239D_S267V_H268D (Variant 32282);
(xviii) the first Fc polypeptide comprises the mutations L235D_G236N_G237A, and the second Fc polypeptide comprises the mutations Template 7+G236D_G237F_S239D_S267V_H268D (Variant 32284);
(xix) the first Fc polypeptide comprises the mutations G236N_G237A_S239G, and the second Fc polypeptide comprises the mutations Template 7+G236D_G237F_S239D_S267V_H268D (Variant 32287);
(xx) the first Fc polypeptide comprises the mutations G236N_G237A_S239H, and the second Fc polypeptide comprises the mutations Template 7+G236D_G237F_S239D_S267V_H268D (Variant 32288);
(xxi) the first Fc polypeptide comprises the mutations G236N_G237E, and the second Fc polypeptide comprises the mutations Template 7+G236D_G237F_S239D_S267V_H268D (Variant 32296);
(xxii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(xxiii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(xxiv) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(xxv) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294), or
(xxvi) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

39.-40. (canceled)

41. The heterodimeric Fc variant according to claim 19, wherein the first Fc polypeptide further comprises a mutation at one or more positions selected from 234, 268, 327, 330 and 331, and wherein:
(i) the mutation at position 234 is selected from L234A, L234F, L234G, L234H, L234I, L234N, L234P, L234Q, L234S, L234T, L234V, L234W and L234Y,
(ii) the mutation at position 268 is selected from H268A, H268D, H268E, H268F, H268G, H268I, H268K, H268L, H268N, H268P, H268Q, H268R, H268S, H268T, H268V, H268W and H268Y,
(iii) the mutation at position 327 is selected from A327E and A327G;
(iv) the mutation at position 330 is selected from A330K, A330H, A330Q, A330R, A330S and A330T, and
(v) the mutation at position 331 is selected from P331A, P331D, P331E, P331H, P331Q and P331S.

42. The heterodimeric Fc variant according to claim 41, wherein the second Fc polypeptide further comprises the mutation S267A or S267Q.

43. The heterodimeric Fc variant according to claim 41, wherein the second Fc polypeptide further comprises the mutation V266L.

44. (canceled)

45. The heterodimeric Fc variant according to claim 41, wherein the first Fc polypeptide further comprises a mutation at one or more of positions 235, 237, 239, 264, 266, 267, 269, 270, 271, 272, 273, 323, 326 and/or 332, and wherein:
(i) the mutation at position 235 is selected from L235A, L235D, L235E, L235F, L235H, L235I, L235P, L235Q, L235S, L235T, L235V, L235W and L235Y;
(ii) the mutation at position 237 is selected from G237A, G237F, G237L, G237N, G237T, G237W and G237Y;
(iii) the mutation at position 239 is selected from S239A, S239D, S239E, S239G, S239I, S239L, S239N, S239Q, S239R and S239V;
(iv) the mutation at position 264 is selected from V264A, V264F, V264I, V264L and V264T;
(v) the mutation at position 266 is V266I;
(vi) the mutation at position 267 is selected from S267A, S267G, S267H, S267I, S267N, S267P, S267T and S267V;
(vii) the mutation at position 269 is selected from E269A, E269D, E269F, E269G, E269H, E269I, E269K, E269L, E269N, E269P, E269Q, E269R, E269S, E269T, E269V, E269W and E269Y;
(viii) the mutation at position 270 is selected from D270A, D270E, D270F, D270H, D270I, D270N, D270Q, D270S, D270T, D270W and D270Y;
(ix) the mutation at position 271 is selected from P271D, P271E, P271G, P271H, P271I, P271K, P271L, P271N, P271Q, P271R, P271V and P271W;
(x) the mutation at position 272 is selected from E272A, E272D, E272F, E272G, E272H, E272I, E272L, E272N, E272S, E272T, E272V, E272W and E272Y;
(xi) the mutation at position 273 is V273A;
(xii) the mutation at position 323 is selected from V323A, V323I and V323L;
(xiii) the mutation at position 326 is selected from K326A, K326D, K326H, K326N, K326Q, K326R, K326S and K326T, and
(xiv) the mutation at position 332 is selected from I332A, I332L, I332T and I332V.

46. (canceled)

47. The heterodimeric Fc variant according to claim 41, wherein the second Fc polypeptide further comprises a mutation at one or more positions selected from 234, 235, 237, 240, 264, 269, 271, 272 and 273, and wherein:
(i) the mutation at position 234 is selected from L234A, L234D, L234E, L234F, L234G, L234I, L234N, L234P, L234Q, L234S, L234T, L234V, L234W and L234Y;
(ii) the mutation at position 235 is selected from L235A, L235D, L235F, L235G, L235H, L235N, L235W and L235Y;
(iii) the mutation at position 237 is selected from G237A, G237D, G237E, G237F, G237H, G237I, G237K, G237L, G237N, G237Q, G237R, G237S, G237T, G237V, G237W and G237Y.
(iv) the mutation at position 240 is selected from V240I, V240L and V240T;
(v) the mutation at position 264 is selected from V264L and V264T;
(vi) the mutation at position 269 is selected from E269D, E269T and E269V;
(vii) the mutation at position 271 is P271G;
(viii) the mutation at position 272 is selected from E272A, E272D, E272I, E272K, E272L, E272P, E272Q, E272R, E272T and E272V, and
(ix) the mutation at position 273 is selected from V273A, V273I, V273L and V273T.

48. The heterodimeric Fc variant according to claim 41, wherein amino acids 325 to 331 in the second Fc polypeptide are replaced with a polypeptide between 8 and 15 amino acids in length.

49. The heterodimeric Fc variant according to claim 19, wherein the heterodimeric Fc variant comprises the amino acid mutations as set out for any one of the variants shown in Table 6.23 or 6.26.

50. The heterodimeric Fc variant according to claim 19, wherein:
(i) the first Fc polypeptide comprises the mutations L234F_L235D_G236N_H268Q_A327G_A330K_P331S, and the second Fc polypeptide comprises the mutations G236D_G237L_S239D_V266L_S267A_H268D (Variant 31190);
(ii) the first Fc polypeptide comprises the mutations L234F_G236N_H268Q_A327G_P329I_A330K_P331S, and the second Fc polypeptide comprises the mutations G236D_G237D_S239D_V266L_S267A_H268D (Variant 31256);
(iii) the first Fc polypeptide comprises the mutations L234F_G236N_H268Q_A327G_P329A_A330K_P331S, and the second Fc polypeptide comprises the mutations G236D_G237L_S239D_V266L_S267A_H268D (Variant 32274);
(iv) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(v) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(vi) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(vii) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294); or
(viii) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

51. The heterodimeric Fc variant according to claim 19, wherein:
(a) the first Fc polypeptide comprises the mutation G236N, and a mutation at one or more positions selected from 234, 268, 327, 330 and 331, wherein:
(i) the mutation at position 234 is selected from L234A, L234F, L234G, L234H, L234I, L234N, L234P, L234Q, L234S, L234T, L234V, L234W and L234Y,
(ii) the mutation at position 268 is selected from H268A, H268D, H268E, H268F, H268G, H268I, H268K, H268L, H268N, H268P, H268Q, H268R, H268S, H268T, H268V, H268W and H268Y,
(iii) the mutation at position 327 is selected from A327G and A327E;
(iv) the mutation at position 330 is selected from A330K, A330H, A330Q, A330R, A330S and A330T, and
(v) the mutation at position 331 is selected from P331A, P331D, P331E, P331H, P331Q and P331S, and
(b) the second Fc polypeptide comprises:
(i) the mutation G236D;
(ii) replacement of the native loop at positions 325 to 331 with a polypeptide of between 8 and 15 amino acids in length, wherein the polypeptide is derived from a loop-forming segment of a second protein, and wherein the loop-forming segment comprises an amino acid sequence as set forth in any one of SEQ ID NOs: 4, 5, 6, 7, 8, 9, 10, 11, 12, 13 or 14, or a variant thereof comprising 1, 2, 3, 4 or 5 amino acid mutations, and
(iii) one or more mutations selected from S239D, S239E, V266I, S267I, S267Q, S267V and H268D.

52. The heterodimeric Fc variant according to claim 51, wherein:
(i) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D (Variant 31192);
(ii) the first Fc polypeptide comprises the mutations L234F_L235D_G236N_H268Q_A327G_A330K_P331S, and the second Fc polypeptide comprises the mutations Template 1 (D329*I)+G236D_G237F_S239D_S267V_H268D_I332L (Variant 32292);
(iii) the first Fc polypeptide comprises the mutations L234F_G236N_S267A_H268Q_A327G_A330K_P331S, and the second Fc polypeptide comprises the mutations Template 1 (D329*I)+G236D_G237F_S239D_S267V_H268D_I332L (Variant 32293);
(iv) the first Fc polypeptide comprises the mutations L234F_G236N_H268Q_A327G_A330T_P331S, and the second Fc polypeptide comprises the mutations Template 1 (D329*I)+G236D_G237F_S239D_S267V_H268D_I332L (Variant 32294); or
(v) the first Fc polypeptide comprises the mutations L234F_G236N_H268Q_A327G_P329I_A330K_P331S, and the second Fc polypeptide comprises the mutations Template 1 (D329*I)+G236D_G237F_S239D_S267V_H268D_I332L (Variant 32295).

53. The heterodimeric Fc variant according to claim 19, wherein the heterodimeric Fc variant comprises the amino acid mutations as set out for any one of the variants shown in Table 13.1.

54. The heterodimeric Fc variant according to claim 3, wherein the first Fc polypeptide and second Fc polypeptide further comprise one or more mutations selected from: A287F, T250V, L309Q and M428F.

55. The heterodimeric Fc variant according to claim 54, wherein the first Fc polypeptide and second Fc polypeptide further comprise the mutations A287F/M428F, A287F/T250V, M428F/T250V or T250V/L309Q.

56. The heterodimeric Fc variant according to claim 3, wherein the heterodimeric Fc variant is a variant of an IgG1 Fc.

57. The heterodimeric Fc variant according to claim 56, wherein the heterodimeric Fc variant is a variant of a human IgG1 Fc.

58. The heterodimeric Fc variant according to claim 3, wherein the selectivity of binding to FcγRIIb of the heterodimeric Fc variant is increased by at least 1.5-fold over the parental Fc region, and wherein:
Fold Difference in FcγRIIb Selectivity=Fold Difference in FcγRIIb Affinity/Fold Difference in FcγRIIaR Affinity,
wherein:
Fold Difference in FcγRIIb Affinity=K_{D} FcγRIIb (parental)/K_{D} FcγRIIb (variant), and
Fold Difference in FcγRIIaR Affinity=K_{D} FcγRIIaR (parental)/K_{D} FcγRIIaR (variant).

59. The heterodimeric Fc variant according to claim 3, wherein the heterodimeric Fc variant has increased binding affinity for FcγRIIb as compared to the parental Fc region.

60. The heterodimeric Fc variant according to claim 59, wherein the binding affinity of the heterodimeric Fc variant for FcγRIIb is increased by at least 10-fold over the parental Fc region, and wherein:
Fold Difference in FcγRIIb Affinity=K_{D} FcγRIIb (parental)/K_{D} FcγRIIb (variant).

61. A polypeptide comprising the heterodimeric Fc variant according to claim 3, and one or more proteinaceous moieties fused or covalently attached to the heterodimeric Fc variant.

62. The polypeptide according to claim 61, wherein the polypeptide is an antibody and the one or more proteinaceous moieties are one or more antigen-binding domains.

63. The polypeptide according to claim 62, wherein at least one of the antigen-binding domains binds to a tumour-associated antigen or tumour-specific antigen.

64. A pharmaceutical composition comprising the heterodimeric Fc variant according to claim 3, and a pharmaceutically acceptable carrier or diluent.

65.-66. (canceled)

67. A method of treating cancer in a subject in need thereof, the method comprising administering to the subject an effective amount of a polypeptide according to claim 63.

68. Nucleic acid encoding the heterodimeric Fc variant according to claim 3.

69. A host cell comprising the nucleic acid according to claim 68.

70. A method of preparing the heterodimeric Fc variant according to claim 3, the method comprising expressing nucleic acid encoding the heterodimeric Fc variant or the polypeptide in a host cell.

71. The heterodimeric Fc variant according to claim 41, wherein the second Fc polypeptide further comprises one or mutations selected from S239D, S239E, V266I, V266L, S267A, S267I, S267V, S267Q and H268D.


SEQUENCE LISTING (186 sequences)

SEQ ID NO: 1 - protein, 217 aa, Artificial Sequence
  Human IgG1 Fc sequence 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 231 aa, Artificial Sequence
  Heavy chain A, upper hinge, CH2 and CH3 domains
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 3 - protein, 231 aa, Artificial Sequence
  Heavy chain B, upper hinge, CH2 and CH3 domains
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 4 - protein, 9 aa, Artificial Sequence
  Template 231 1QVC (Bacteria)
WTDQSGQDR

SEQ ID NO: 5 - protein, 9 aa, Artificial Sequence
  Template 168 1LN1 (Human)
LDMEGRKIH

SEQ ID NO: 6 - protein, 10 aa, Artificial Sequence
  Template 1 2GKO (Parental)
STWFDGGYAT

SEQ ID NO: 7 - protein, 10 aa, Artificial Sequence
  Template 11 2DWC
HFDENGEIVT

SEQ ID NO: 8 - protein, 10 aa, Artificial Sequence
  Template 7 4R3O (Human - Parental)
GLDEEGKGAV

SEQ ID NO: 9 - protein, 10 aa, Artificial Sequence
  Template 19 1OID (Bacteria - Parental)
VTWEDGKSER

SEQ ID NO: 10 - protein, 10 aa, Artificial Sequence
  Template 38 3GVE (Bacteria)
LIDENGNEQK

SEQ ID NO: 11 - protein, 10 aa, Artificial Sequence
  Template 60 3E35 (Bacteria)
VQDATGAPFL

SEQ ID NO: 12 - protein, 10 aa, Artificial Sequence
  Template 66 1IJR (Human - Parental)
DFDQNQGEVV

SEQ ID NO: 13 - protein, 9 aa, Artificial Sequence
  Template 83 2X6C (Bacteria)
SDFEGKPTL

SEQ ID NO: 14 - protein, 10 aa, Artificial Sequence
  Template 151 4JN3 (Bacteria - Parental)
LTDEEGRPYR

SEQ ID NO: 15 - protein, 10 aa, Artificial Sequence
  Template 1 (S325*A_ T326*H_ F328*E_ D329*G_ A331*BN)
AHWEGGGYNT

SEQ ID NO: 16 - protein, 10 aa, Artificial Sequence
  Template 1 (S325*A_ T326*H_ F328*Q_ D329*G_ A331*BN)
AHWQGGGYNT

SEQ ID NO: 17 - protein, 10 aa, Artificial Sequence
  Template 1 (T326*Q)
SQWFDGGYAT

SEQ ID NO: 18 - protein, 10 aa, Artificial Sequence
  Template 1 (T326*N)
SNWFDGGYAT

SEQ ID NO: 19 - protein, 10 aa, Artificial Sequence
  Template 1 (Y331*AF)
STWFDGGFAT

SEQ ID NO: 20 - protein, 10 aa, Artificial Sequence
  Template 1 (G330*E)
STWFDEGYAT

SEQ ID NO: 21 - protein, 10 aa, Artificial Sequence
  Template 1 (G330*D)
STWFDDGYAT

SEQ ID NO: 22 - protein, 10 aa, Artificial Sequence
  Template 1 (G330*A)
STWFDAGYAT

SEQ ID NO: 23 - protein, 10 aa, Artificial Sequence
  Template 1 (T326*L)
SLWFDGGYAT

SEQ ID NO: 24 - protein, 10 aa, Artificial Sequence
  Template 1 (T326*I)
SIWFDGGYAT

SEQ ID NO: 25 - protein, 10 aa, Artificial Sequence
  Template 1 (T326*E)
SEWFDGGYAT

SEQ ID NO: 26 - protein, 10 aa, Artificial Sequence
  Template 1 (T326*D)
SDWFDGGYAT

SEQ ID NO: 27 - protein, 10 aa, Artificial Sequence
  Template 1 (T326*A)
SAWFDGGYAT

SEQ ID NO: 28 - protein, 10 aa, Artificial Sequence
  Template 1 (T326*F)
SFWFDGGYAT

SEQ ID NO: 29 - protein, 10 aa, Artificial Sequence
  Template 1 (T326*H_ F328*T)
SHWTDGGYAT

SEQ ID NO: 30 - protein, 10 aa, Artificial Sequence
  Template 1 (T326*H_ F328*S_ A331*BN)
SHWSDGGYNT

SEQ ID NO: 31 - protein, 10 aa, Artificial Sequence
  Template 1 (T326*H_ F328*S)
SHWSDGGYAT

SEQ ID NO: 32 - protein, 10 aa, Artificial Sequence
  Template 1 (T326*H_ F328*Q_ D329*G_ A331*BN)
SHWQGGGYNT

SEQ ID NO: 33 - protein, 10 aa, Artificial Sequence
  Template 1 (T326*H_ F328*Q_ D329*G)
SHWQGGGYAT

SEQ ID NO: 34 - protein, 10 aa, Artificial Sequence
  Template 1 (T326*H_ F328*Q)
SHWQDGGYAT

SEQ ID NO: 35 - protein, 10 aa, Artificial Sequence
  Template 1 (T326*H_ F328*N)
SHWNDGGYAT

SEQ ID NO: 36 - protein, 10 aa, Artificial Sequence
  Template 1 (T326*H_ F328*H_ D329*G)
SHWHGGGYAT

SEQ ID NO: 37 - protein, 10 aa, Artificial Sequence
  Template 1 (T326*H_ F328*H)
SHWHDGGYAT

SEQ ID NO: 38 - protein, 10 aa, Artificial Sequence
  Template 1 (T326*H)
SHWFDGGYAT

SEQ ID NO: 39 - protein, 10 aa, Artificial Sequence
  Template 1 (T326*H_ F328*E_ D329*G_ A331*BN)
SHWEGGGYNT

SEQ ID NO: 40 - protein, 10 aa, Artificial Sequence
  Template 1 (T326*H_ F328*E_ D329*G)
SHWEGGGYAT

SEQ ID NO: 41 - protein, 10 aa, Artificial Sequence
  Template 1 (T326*H_ F328*D)
SHWDDGGYAT

SEQ ID NO: 42 - protein, 10 aa, Artificial Sequence
  Template 1 (T326*H_ A331*BN)
SHWFDGGYNT

SEQ ID NO: 43 - protein, 10 aa, Artificial Sequence
  Template 1 (F328*H_ D329*G)
STWHGGGYAT

SEQ ID NO: 44 - protein, 10 aa, Artificial Sequence
  Template 1 (F328*H)
STWHDGGYAT

SEQ ID NO: 45 - protein, 10 aa, Artificial Sequence
  Template 1 (F328*Q_ D329*G)
STWQGGGYAT

SEQ ID NO: 46 - protein, 10 aa, Artificial Sequence
  Template 1 (D329*L)
STWFLGGYAT

SEQ ID NO: 47 - protein, 10 aa, Artificial Sequence
  Template 1 (D329*I)
STWFIGGYAT

SEQ ID NO: 48 - protein, 10 aa, Artificial Sequence
  Template 1 (F328*S)
STWSDGGYAT

SEQ ID NO: 49 - protein, 10 aa, Artificial Sequence
  Template 1 (F328*S_ A331*BN)
STWSDGGYNT

SEQ ID NO: 50 - protein, 10 aa, Artificial Sequence
  Template 1 (S325*A_ T326*H)
AHWFDGGYAT

SEQ ID NO: 51 - protein, 10 aa, Artificial Sequence
  Template 1 (S325*A_ T326*H_ A331*BN)
AHWFDGGYNT

SEQ ID NO: 52 - protein, 10 aa, Artificial Sequence
  Template 1 (S325*A_ T326*H_ F328*Q_ D329*G)
AHWQGGGYAT

SEQ ID NO: 53 - protein, 10 aa, Artificial Sequence
  Template 1 (S325*A_ T326*H_ F328*S)
AHWSDGGYAT

SEQ ID NO: 54 - protein, 10 aa, Artificial Sequence
  Template 1 (S325*A_ T326*H_ F328*S_ A331*BN)
AHWSDGGYNT

SEQ ID NO: 55 - protein, 10 aa, Artificial Sequence
  Template 1 (S325*A)
ATWFDGGYAT

SEQ ID NO: 56 - protein, 10 aa, Artificial Sequence
  Template 1 (S325*A_ A331*BN)
ATWFDGGYNT

SEQ ID NO: 57 - protein, 10 aa, Artificial Sequence
  Template 1 (T326*W)
SWWFDGGYAT

SEQ ID NO: 58 - protein, 10 aa, Artificial Sequence
  Template 1 (T326*V)
SVWFDGGYAT

SEQ ID NO: 59 - protein, 10 aa, Artificial Sequence
  Template 1 (F328*Y)
STWYDGGYAT

SEQ ID NO: 60 - protein, 10 aa, Artificial Sequence
  Template 1 (F328*S_ D329*G)
STWSGGGYAT

SEQ ID NO: 61 - protein, 10 aa, Artificial Sequence
  Template 1 (D329*G)
STWFGGGYAT

SEQ ID NO: 62 - protein, 10 aa, Artificial Sequence
  Template 1 (S325*A_ F328*S_ A331*BN)
ATWSDGGYNT

SEQ ID NO: 63 - protein, 10 aa, Artificial Sequence
  Template 1 (A331*BL)
STWFDGGYLT

SEQ ID NO: 64 - protein, 10 aa, Artificial Sequence
  Template 1 (A331*BK)
STWFDGGYKT

SEQ ID NO: 65 - protein, 10 aa, Artificial Sequence
  Template 1 (A331*BH)
STWFDGGYHT

SEQ ID NO: 66 - protein, 10 aa, Artificial Sequence
  Template 1 (A331*BF)
STWFDGGYFT

SEQ ID NO: 67 - protein, 10 aa, Artificial Sequence
  Template 1 (Y331*AW)
STWFDGGWAT

SEQ ID NO: 68 - protein, 10 aa, Artificial Sequence
  Template 1 (D330*K)
STWFDKGYAT

SEQ ID NO: 69 - protein, 10 aa, Artificial Sequence
  Template 1 (G330*R)
STWFDRGYAT

SEQ ID NO: 70 - protein, 10 aa, Artificial Sequence
  Template 1 (G330*H)
STWFDHGYAT

SEQ ID NO: 71 - protein, 10 aa, Artificial Sequence
  Template 7 (E328*Q_ E329*S_ A331*BY)
GLDQSGKGYV

SEQ ID NO: 72 - protein, 10 aa, Artificial Sequence
  Template 7 (E328*T_ E329*N_ A331*BY)
GLDTNGKGYV

SEQ ID NO: 73 - protein, 10 aa, Artificial Sequence
  Template 7 (E328*H_ E329*R_ A331*BY)
GLDHRGKGYV

SEQ ID NO: 74 - protein, 10 aa, Artificial Sequence
  Template 7 (G325*F_ E328*T_ E329*N_ A331*BV)
FLDTNGKGVV

SEQ ID NO: 75 - protein, 10 aa, Artificial Sequence
  Template 7 (G325*F_ E328*Q_ E329*S_ A331*BV)
FLDQSGKGVV

SEQ ID NO: 76 - protein, 10 aa, Artificial Sequence
  Template 7 (G325*F_ E328*H_ E329*R_ A331*BV)
FLDHRGKGVV

SEQ ID NO: 77 - protein, 10 aa, Artificial Sequence
  Template 7 (G325*F_ E329*N_ A331*BV)
FLDENGKGVV

SEQ ID NO: 78 - protein, 10 aa, Artificial Sequence
  Template 7 (E329*N_ A331*BY)
GLDENGKGYV

SEQ ID NO: 79 - protein, 10 aa, Artificial Sequence
  Template 66 (D327*N_ Q328*H_ Q330*D)
DFNHNDGEVV

SEQ ID NO: 80 - protein, 10 aa, Artificial Sequence
  Template 66 (Q328*T_ N329*D_ Q330*D)
DFDTDDGEVV

SEQ ID NO: 81 - protein, 10 aa, Artificial Sequence
  Template 66 (Q328*S_ N329*T)
DFDSTQGEVV

SEQ ID NO: 82 - protein, 10 aa, Artificial Sequence
  Template 66 (Q328*S_ N329*T_ Q330*D)
DFDSTDGEVV

SEQ ID NO: 83 - protein, 10 aa, Artificial Sequence
  Template 66 (Q328*T_ N329*S)
DFDTSQGEVV

SEQ ID NO: 84 - protein, 10 aa, Artificial Sequence
  Template 66 (Q328*H_ N329*D)
DFDHDQGEVV

SEQ ID NO: 85 - protein, 10 aa, Artificial Sequence
  Template 66 (Q328*N_ N329*D_ Q330*D)
DFDNDDGEVV

SEQ ID NO: 86 - protein, 10 aa, Artificial Sequence
  Template 66 (Q328*E_ N329*D_ Q330*D)
DFDEDDGEVV

SEQ ID NO: 87 - protein, 10 aa, Artificial Sequence
  Template 151 (E328*H_ E329*N_ Y331*BI)
LTDHNGRPIR

SEQ ID NO: 88 - protein, 10 aa, Artificial Sequence
  Template 151 (Y331*BI)
LTDEEGRPIR

SEQ ID NO: 89 - protein, 10 aa, Artificial Sequence
  Template 151 (E329*D_ R331*S_ Y331*BI)
LTDEDGSPIR

SEQ ID NO: 90 - protein, 10 aa, Artificial Sequence
  Template 151 (E329*D_ Y331*BI)
LTDEDGRPIR

SEQ ID NO: 91 - protein, 10 aa, Artificial Sequence
  Template 1 (T326*S)
SSWFDGGYAT

SEQ ID NO: 92 - protein, 10 aa, Artificial Sequence
  Template 1 (Y331*AE)
STWFDGGEAT

SEQ ID NO: 93 - protein, 10 aa, Artificial Sequence
  Template 1 (Y331*AD)
STWFDGGDAT

SEQ ID NO: 94 - protein, 10 aa, Artificial Sequence
  Template 1 (Y331*AA)
STWFDGGAAT

SEQ ID NO: 95 - protein, 10 aa, Artificial Sequence
  Template 1 (F328*A)
STWADGGYAT

SEQ ID NO: 96 - protein, 10 aa, Artificial Sequence
  Template 1 (W327*T_ F328*H_ D329*G)
STTHGGGYAT

SEQ ID NO: 97 - protein, 10 aa, Artificial Sequence
  Template 1 (S325*N)
NTWFDGGYAT

SEQ ID NO: 98 - protein, 10 aa, Artificial Sequence
  Template 1 (Y331*AH)
STWFDGGHAT

SEQ ID NO: 99 - protein, 10 aa, Artificial Sequence
  Template 1 (T326*H_ F328*E_ A331*BN)
SHWEDGGYNT

SEQ ID NO: 100 - protein, 10 aa, Artificial Sequence
  Template 1 (T326*H_ F328*E)
SHWEDGGYAT

SEQ ID NO: 101 - protein, 10 aa, Artificial Sequence
  Template 1 (S325*D)
DTWFDGGYAT

SEQ ID NO: 102 - protein, 10 aa, Artificial Sequence
  Template 1 (T326*H_ W327*T_ F328*T_ D329*G)
SHTTGGGYAT

SEQ ID NO: 103 - protein, 10 aa, Artificial Sequence
  Template 1 (T326*H_ W327*T_ F328*H_ D329*G)
SHTHGGGYAT

SEQ ID NO: 104 - protein, 10 aa, Artificial Sequence
  Template 1 (T326*H_ W327*D_ F328*T_ D329*G)
SHDTGGGYAT

SEQ ID NO: 105 - protein, 10 aa, Artificial Sequence
  Template 1 (F328*I)
STWIDGGYAT

SEQ ID NO: 106 - protein, 10 aa, Artificial Sequence
  Template 1 (D329*Q)
STWFQGGYAT

SEQ ID NO: 107 - protein, 10 aa, Artificial Sequence
  Template 1 (D329*P)
STWFPGGYAT

SEQ ID NO: 108 - protein, 10 aa, Artificial Sequence
  Template 1 (S325*A_ T326*H_ F328*E_ A331*BN)
AHWEDGGYNT

SEQ ID NO: 109 - protein, 10 aa, Artificial Sequence
  Template 1 (S325*A_ T326*H_ F328*E_ D329*G)
AHWEGGGYAT

SEQ ID NO: 110 - protein, 10 aa, Artificial Sequence
  Template 1 (T326*Y)
SYWFDGGYAT

SEQ ID NO: 111 - protein, 10 aa, Artificial Sequence
  Template 1 (F328*W)
STWWDGGYAT

SEQ ID NO: 112 - protein, 10 aa, Artificial Sequence
  Template 1 (F328*V)
STWVDGGYAT

SEQ ID NO: 113 - protein, 10 aa, Artificial Sequence
  Template 1 (F328*T)
STWTDGGYAT

SEQ ID NO: 114 - protein, 10 aa, Artificial Sequence
  Template 1 (D329*E)
STWFEGGYAT

SEQ ID NO: 115 - protein, 10 aa, Artificial Sequence
  Template 1 (A331*BR)
STWFDGGYRT

SEQ ID NO: 116 - protein, 10 aa, Artificial Sequence
  Template 1 (A331*BQ)
STWFDGGYQT

SEQ ID NO: 117 - protein, 10 aa, Artificial Sequence
  Template 1 (A331*BN)
STWFDGGYNT

SEQ ID NO: 118 - protein, 10 aa, Artificial Sequence
  Template 1 (A331*BG)
STWFDGGYGT

SEQ ID NO: 119 - protein, 10 aa, Artificial Sequence
  Template 1 (A331*BE)
STWFDGGYET

SEQ ID NO: 120 - protein, 10 aa, Artificial Sequence
  Template 1 (A331*BD)
STWFDGGYDT

SEQ ID NO: 121 - protein, 10 aa, Artificial Sequence
  Template 1 (Y331*AP)
STWFDGGPAT

SEQ ID NO: 122 - protein, 10 aa, Artificial Sequence
  Template 1 (G330*S)
STWFDSGYAT

SEQ ID NO: 123 - protein, 10 aa, Artificial Sequence
  Template 1 (G330*N)
STWFDNGYAT

SEQ ID NO: 124 - protein, 10 aa, Artificial Sequence
  Template 1 (G330*T)
STWFDTGYAT

SEQ ID NO: 125 - protein, 10 aa, Artificial Sequence
  Template 1 (G330*Y)
STWFDYGYAT

SEQ ID NO: 126 - protein, 10 aa, Artificial Sequence
  Template 7 (G325*F_ A330*BV)
FLDEEGKGVV

SEQ ID NO: 127 - protein, 10 aa, Artificial Sequence
  Template 7 (E328*Q_ E329*S_ A330*BV)
GLDQSGKGVV

SEQ ID NO: 128 - protein, 10 aa, Artificial Sequence
  Template 7 (E328*Q_ E329*S)
GLDQSGKGAV

SEQ ID NO: 129 - protein, 10 aa, Artificial Sequence
  Template 7 (E328*H_ E329*T)
GLDHTGKGAV

SEQ ID NO: 130 - protein, 10 aa, Artificial Sequence
  Template 7 (E328*T_ E329*N_ A330*BV)
GLDTNGKGVV

SEQ ID NO: 131 - protein, 10 aa, Artificial Sequence
  Template 7 (E328*T_ E329*N)
GLDTNGKGAV

SEQ ID NO: 132 - protein, 10 aa, Artificial Sequence
  Template 7 (G325*F)
FLDEEGKGAV

SEQ ID NO: 133 - protein, 10 aa, Artificial Sequence
  Template 7 (E328*H_ E329*R)
GLDHRGKGAV

SEQ ID NO: 134 - protein, 10 aa, Artificial Sequence
  Template 7 (G325*F_ E328*T_ E329*N)
FLDTNGKGAV

SEQ ID NO: 135 - protein, 10 aa, Artificial Sequence
  Template 7 (G325*F_ E328*Q_ E329*S)
FLDQSGKGAV

SEQ ID NO: 136 - protein, 10 aa, Artificial Sequence
  Template 7 (G325*F_ E328*H_ E329*R)
FLDHRGKGAV

SEQ ID NO: 137 - protein, 10 aa, Artificial Sequence
  Template 7 (G325*F_ E329*N)
FLDENGKGAV

SEQ ID NO: 138 - protein, 10 aa, Artificial Sequence
  Template 7 (E328*H_ E329*R_ A331*BV)
GLDHRGKGVV

SEQ ID NO: 139 - protein, 10 aa, Artificial Sequence
  Template 7 (E329*N_ A331*BV)
GLDENGKGVV

SEQ ID NO: 140 - protein, 10 aa, Artificial Sequence
  Template 7 (E329*N)
GLDENGKGAV

SEQ ID NO: 141 - protein, 10 aa, Artificial Sequence
  Template 7 (A331*BY)
GLDEEGKGYV

SEQ ID NO: 142 - protein, 10 aa, Artificial Sequence
  Template 7 (A331*BV)
GLDEEGKGVV

SEQ ID NO: 143 - protein, 10 aa, Artificial Sequence
  Template 7 - strat3-HF (Parental)
GTDEEGKGAT

SEQ ID NO: 144 - protein, 10 aa, Artificial Sequence
  Template 7-HF (E329*N_ A331*BV)
GTDENGKGVT

SEQ ID NO: 145 - protein, 10 aa, Artificial Sequence
  Template 7-HF (E329*N)
GTDENGKGAT

SEQ ID NO: 146 - protein, 10 aa, Artificial Sequence
  Template 7-HF (A331*BV)
GTDEEGKGVT

SEQ ID NO: 147 - protein, 10 aa, Artificial Sequence
  Template 7-HF (G325*F_ A331*BV)
FTDEEGKGVT

SEQ ID NO: 148 - protein, 10 aa, Artificial Sequence
  Template 7-HF (G325*F)
FTDEEGKGAT

SEQ ID NO: 149 - protein, 10 aa, Artificial Sequence
  Template 66 (D325*A_ Q328*P_ N329*D)
AFDPDQGEVV

SEQ ID NO: 150 - protein, 10 aa, Artificial Sequence
  Template 66 (D325*A_ Q328*D_ N329*E_ Q330*D)
AFDDEDGEVV

SEQ ID NO: 151 - protein, 10 aa, Artificial Sequence
  Template 66 (D327*N_ Q328*D_ N329*E)
DFNDEQGEVV

SEQ ID NO: 152 - protein, 10 aa, Artificial Sequence
  Template 66 (Q328*E_ N329*E)
DFDEEQGEVV

SEQ ID NO: 153 - protein, 10 aa, Artificial Sequence
  Template 66 (D325*A_ Q328*E_ N329*D_ Q330*D)
AFDEDDGEVV

SEQ ID NO: 154 - protein, 10 aa, Artificial Sequence
  Template 66 (D325*A_ Q328*E_ N329*E)
AFDEEQGEVV

SEQ ID NO: 155 - protein, 10 aa, Artificial Sequence
  Template 66 (D325*A_ Q328*H_ N329*D)
AFDHDQGEVV

SEQ ID NO: 156 - protein, 10 aa, Artificial Sequence
  Template 66 (D325*A)
AFDQNQGEVV

SEQ ID NO: 157 - protein, 10 aa, Artificial Sequence
  Template 66 (D325*A_ Q328*S_ N329*T_ Q330*D)
AFDSTDGEVV

SEQ ID NO: 158 - protein, 10 aa, Artificial Sequence
  Template 66 (Q328*D_ N329*S)
DFDDSQGEVV

SEQ ID NO: 159 - protein, 10 aa, Artificial Sequence
  Template 66 (Q328*D_ N329*E_ Q330*D)
DFDDEDGEVV

SEQ ID NO: 160 - protein, 10 aa, Artificial Sequence
  Template 66 (Q328*P_ N329*D)
DFDPDQGEVV

SEQ ID NO: 161 - protein, 10 aa, Artificial Sequence
  Template 66 - strat3-HF (Parental)
DTDQNQGEVT

SEQ ID NO: 162 - protein, 10 aa, Artificial Sequence
  Template 66-HF (Q328*D_ N329*E_ Q330*D)
DTDDEDGEVT

SEQ ID NO: 163 - protein, 10 aa, Artificial Sequence
  Template 66-HF (D325*A_ Q328*D_ N329*E_ Q330*D)
ATDDEDGEVT

SEQ ID NO: 164 - protein, 10 aa, Artificial Sequence
  Template 66-HF (D325*A_ Q328*P_ N329*D)
ATDPDQGEVT

SEQ ID NO: 165 - protein, 10 aa, Artificial Sequence
  Template 66-HF (D325*A)
ATDQNQGEVT

SEQ ID NO: 166 - protein, 10 aa, Artificial Sequence
  Template 66-HF (Q328*P_ N329*D)
DTDPDQGEVT

SEQ ID NO: 167 - protein, 10 aa, Artificial Sequence
  Template 151 (R331*S)
LTDEEGSPYR

SEQ ID NO: 168 - protein, 10 aa, Artificial Sequence
  Template 151 (E328*H_ E329*N)
LTDHNGRPYR

SEQ ID NO: 169 - protein, 10 aa, Artificial Sequence
  Template 151 (E329*D_  R331*S)
LTDEDGSPYR

SEQ ID NO: 170 - protein, 10 aa, Artificial Sequence
  Template 151 (E329*D)
LTDEDGRPYR

SEQ ID NO: 171 - protein, 10 aa, Artificial Sequence
  Template 151 (Y331*BQ)
LTDEEGRPQR

SEQ ID NO: 172 - protein, 10 aa, Artificial Sequence
  Template 19 (V325*A)
ATWEDGKSER

SEQ ID NO: 173 - protein, 10 aa, Artificial Sequence
  Template 356 3A15 (Bacteria)
HIDNQGYENL

SEQ ID NO: 174 - protein, 9 aa, Artificial Sequence
  Template 2552 EQB (Yeast)
VDINGKKVK

SEQ ID NO: 175 - protein, 10 aa, Artificial Sequence
  Template 53 3CIN (Bacteria)
YVSFNGATDE

SEQ ID NO: 176 - protein, 10 aa, Artificial Sequence
  Template 47 1L4U (Lobster)
GIAYDGNLLK

SEQ ID NO: 177 - protein, 10 aa, Artificial Sequence
  Template 5 2W3Y (Bacteria)
FQDTSGNVFY

SEQ ID NO: 178 - protein, 10 aa, Artificial Sequence
  Template 48 2HLC (Insect)
ITLQDQRRVW

SEQ ID NO: 179 - protein, 10 aa, Artificial Sequence
  Template 28 1JJ2 (Archaea)
VEFEDGDRRL

SEQ ID NO: 180 - protein, 11 aa, Artificial Sequence
  Template 82 3LYV (Pathogenic Bacteria)
YTDSEDGATNI

SEQ ID NO: 181 - protein, 11 aa, Artificial Sequence
  Template 3 1JCF (Bacteria)
GIDLSTGLPRK

SEQ ID NO: 182 - protein, 12 aa, Artificial Sequence
  Template 12 14-2
DFTPKAKLGFQI

SEQ ID NO: 183 - protein, 13 aa, Artificial Sequence
  Template 13_3
VLDDPSRENEADL

SEQ ID NO: 184 - protein, 12 aa, Artificial Sequence
  Template 12_14
NFTPKAKLGFEI

SEQ ID NO: 185 - protein, 14 aa, Artificial Sequence
  Template 14_0
QVHEDATKPYGLSL

SEQ ID NO: 186 - protein, 11 aa, Artificial Sequence
  Template 11_14
APQINPHSPKF